(19) United States
(12) Patent Application Publication
(10) Pub. No.: US 2007/0060573 A1
(43) Pub. Date: Mar. 15, 2007

(54) Acyltryptophanols

(76) Inventors: Lars Wortmann, Berlin (DE); Arwed Cleve, Berlin (DE); Bernd Menzenbach, Jena (DE); Hans-Peter Muhn, Berlin (DE); Gernot Langer, Berlin (DE); Anna Schrey, Berlin (DE); Ronald Kuehne, Berlin (DE); Marcu Koppitz, Berlin (DE); Dirk Kosemund, Erfurt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/501,228

(22) Filed: Aug. 9, 2006

Related U.S. Application Data
(60) Provisional application No. 60/706,743, filed on Aug. 10, 2005.

(51) Int. Cl. A61K 31/551; A61K 31/541; A61K 31/5377; A61K 31/496; A61K 31/454; A61K 31/405; C07D 417/02; C07D 413/02; C07D 403/02

(52) U.S. Cl. 514/218; 514/227.8; 514/232.8; 514/254.09; 514/323; 514/414; 540/575; 544/60; 544/373; 546/201; 548/465; 544/143

(57) ABSTRACT

The present patent application relates to acyltryptophanols of the general formula I, 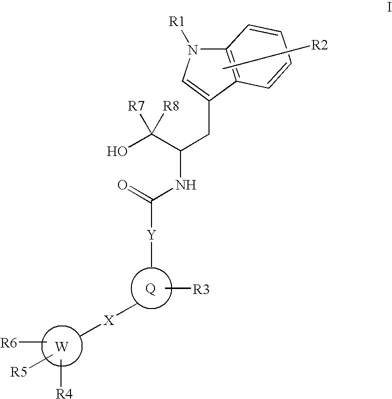 in which Q, X, Y, W, R1, R2, R3, R4, R5, R8 have the meanings stated in the description. The compounds according to the invention are effective FSH antagonists and can be used for example for fertility control in men or in women, or for the prevention and/or treatment of osteoporosis.

This application claims the benefit of the filing date of U.S. Provisional Application Ser. No. 60/706,743 filed Aug. 10, 2005.

The present patent application relates to novel acyltryptophanols, process for their preparation, pharmaceutical compositions comprising the compounds according to the invention, and the use thereof for fertility control in men or in women.

Follicle-stimulating hormone (FSH) and luteinizing hormone (LH) are together responsible for the control of male and female fertility and of the production of sex steroids.

In the female mammal, FSH controls the early ripening of ovarian primary follicles and the biosynthesis of sex steroids. In the advanced stage of differentiation (preantral follicles), the influence of LH becomes increasingly important for further development of the follicles until ovulation occurs.

In male mammals, FSH is primarily responsible for the differentiation and stimulation of Sertoli cells. Their function consists of assisting spermatogenesis on many levels. LH is primarily responsible for stimulating the Leydig cells and thus androgen production. FSH, LH and TSH (thyrotropic hormone) together form the group of glycoprotein hormones which are formed in the pituitary and are secreted from there. Whereas the alpha subunit is common to the three hormones, their specificity of action is determined by the beta chain which is unique in each case. The molecular weight of FSH including the sugar portion is about 30 kD.

FSH and the other glycoprotein hormones act specifically via their selectively expressed G protein-coupled receptor (GPCR). FSH stimulates, through binding to its receptor, the association thereof with a stimulating G protein (Gₛ) which is thereby stimulated to hydrolyse guanosine triphosphate (GTP) and to activate the membrane-associated adenylate cyclase. Cyclic adenosine monophosphate (cAMP) is accordingly an important and readily quantifiable secondary messenger substance of FSH (G. Vassart, L. Pardo, S. Costagliola, Trends Biochem. Sci. 2004, 29, 119-126).

The importance of FSH for male fertility is the subject of intensive research. It has been possible to show that FSH influences several processes of spermatogenesis such as the proliferation of spermatogonia, the antiapoptotic effect on spermatogonia and spermatocytes and the stimulation of sperm maturation including motility thereof.

The following arguments are also in favour of the FSH receptor as target for male fertility control:
1. The FSH receptor is exclusively expressed on Sertoli cells (high specificity).
2. Contraceptive vaccination against FSH beta chain or the FSH receptor induces infertility in male primates (N. R. Mougdal, M. Jeyakumar, H. N. Krishnamurthy, S. Sridhar, H. Krishnamurthy, F. Martin, Human Reproduction Update 1997, 3, 335-346).
3. Naturally occurring mutations in the FSH receptor or the FSH beta chain may lead to sub- or infertility in men (I. Huhtaniemi, Journal of Reproduction and Fertility 2000, 119, 173-186; L. C. Layman, P. G. McDonough, Molecular and Cellular Endocrinology 2000, 161, 9-17).
4. Neutralizing FSH antiserum has no effect on testis weight and testosterone production (V. Sriraman, A. J. Rao, Molecular and Cellular Endocrionology 2004, 224, 73-82). Adverse effects of FSH blockade on androgen production therefore appear unlikely.

In accordance with the stated arguments, it is to be expected that effective FSH antagonists are suitable for spermatogenesis inhibition (prevention) in men. However, a suitable FSH antagonist also leads to infertility in women, because it will suppress follicle ripening and thus also ovulation.

On the other hand, the skilled person expects advantages from non-peptidergic FSH agonists when used to promote fertility in women (stimulation of follicle ripening). There are no reports of experience on the use of FSH or FSH agonists in male infertility, but specific indications are also conceivable in this connection.

New findings demonstrate that there is also a direct effect of FSH on cells of bone metabolism. Two fundamentally different cell types need to be distinguished: osteoclasts play a central role in bone resorption (breakdown of bone). Osteoblasts simulate bone density (anabolic effect).

FSH receptors have been detected in osteoclasts but not osteoblasts. In vitro, FSH stimulates bone resorption by mouse osteoclasts (Li Sun et al. 2006. FSH directly regulates bone mass. Cell 2006; 125: 247-60). A clinical correlation between the height of the serum FSH levels and low bone density has been observed in postmenopausal women (Devleta et al, 2004; Hypergonadotropic amenorrhea and bone density: new approach to an old problem. J. Bone Miner. Metab. 22: 360-4).

This and other findings suggest that FSH stimulates loss of bone mass, and accordingly FSH antagonists will display an antiresorptive effect on bone and are therefore suitable for the therapy and/or prevention of peri- and postmenopausal loss of bone mass and osteoporosis.

In recent years, some low molecular weight FSH receptor modulators, FSH receptor antagonists and FSH receptor agonists from various classes of substances have been published.

FSH receptor modulators are disclosed in WO 2004/056779, WO 2004/056780; J. Med. Chem. 2005, 48, 1697 [Tetrahydroquinolines]; WO 02/70493, Bioorg. Med. Chem. Lett. 2004, 14, 1713 and 1717 [Diketopiperazines]; and WO 01/47875 [Sulphonamides]. FSH receptor agonists are disclosed in WO 02/09706; J. Comb. Chem. 2004, 6, 196 [Thiazolidinones]; WO 2003/020726 and WO 03/20727, Chem. Biochem. 2002, 10, 1023 (Thieno[2,3-d]pyrimidines); WO 01/87287 [Pyrazoles]; WO 00/08015 [Carbazoles]. Examples of FSH receptor antagonists are disclosed in WO 03/004028 [Tetrahydroquinolines], WO 02/09705 [Thiazolidinones], WO 00/58277, Bioorg. Med. Chem. 2002, 10, 639 [Sulphonic acids]; WO 00/58276, Endocr. 2002, 143, 3822; Synth. Comm. 2002, 32, 2695 [Azo compounds].

The object of the present invention was therefore to provide alternative compounds having an FSH receptor antagonistic effect.

The object has been achieved according to the present invention by the compounds of the formula I 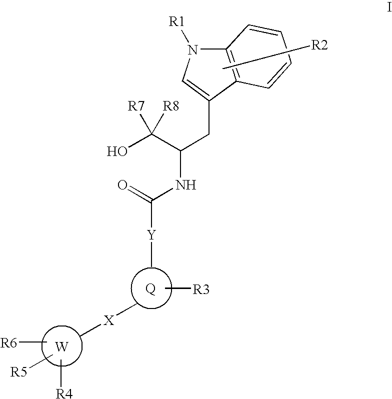
in which
R1 may be hydrogen, C₁-C₆-alkyl, C₃-C₆-alkenyl, C₃-C₆-alkynyl, C₃-C₇-cycloalkyl, C₁-C₆-alkyloxy-C₁-C₆-alkylene, C₃-C₇-cycloalkyloxy-C₁-C₆-alkylene, C₁-C₆-alkylamino-C₁-C₆-alkylene, di(C₁-C₆-alkyl)amino-C₁-C₆-alkylene, phenyloxy-C₁-C₆-alkylene;
   where the hydrocarbon chains therein may optionally be substituted one or more times by fluorine, cyano, hydroxy, amino or the groups: 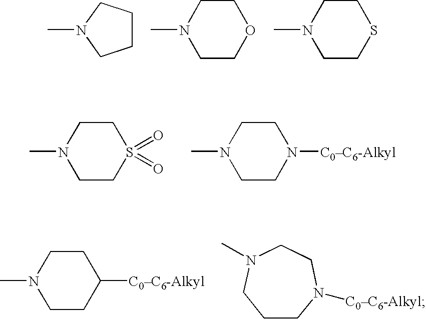
R2 may be hydrogen, halogen, cyano, —SO₂Me, C₁-C₆-alkyl, C₂-C₆-alkenyl, C₂-C₆-alkynyl, C₁-C₆-alkyloxy or benzyloxy,
   where the hydrocarbon chains therein may optionally be substituted one or more times by fluorine;
R3 may be hydrogen, hydroxy, halogen, nitro, amino, cyano, C₁-C₆-alkyl, C₁-C₆-alkenyl or C₁-C₆-alkynyl, C₃-C₇-cycloalkyl, hydroxy-C₁-C₆-alkylene, hydroxy-C₃-C₆-alkenylene, hydroxy-C₃-C₆-alkynylene, C₁-C₆-alkyloxy, C₁-C₆-alkyloxy-C₁-C₆-alkylene, C₃-C₇-cycloalkyloxy, C₃-C₇-cycloalkyl-C₁-C₆-alkylenoxy, C₃-C₇-cycloalkyloxy-C₁-C₆-alkylene, C₁-C₆-alkyloxy-C₃-C₆-alkenylene, C₁-C₆-alkyloxy-C₃-C₆-alkynylene, C₁-C₆-alkyloxyphenyl-C₁-C₆-alkylene, C₁-C₆-alkylamino-C₁₋₆-alkylene, di(C₁-C₆-alkyl)amino-C₁-C₆-alkylene, phenyloxy-C₁-C₆-alkylene;
   where the hydrocarbon chains therein may optionally be substituted one or more times by fluorine, cyano, hydroxy, amino or the groups 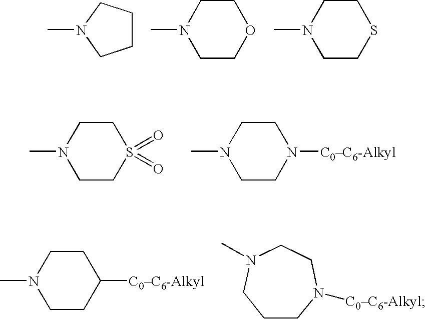
R4, R5, R6 may be independently of one another hydrogen, hydroxy, halogen, nitro, amino, cyano, phenyl, C₁₋₆-alkyl, C₂-C₆-alkenyl or C₂-C₆-alkynyl, C₃-C₇-cycloalkyl, C₃₋₄₇-cycloalkyl-C₁-C₆-alkylene, C₃-C₇-heterocycloalkyl,
   where the hydrocarbon chains therein may optionally be substituted one or more times by fluorine, cyano or the radicals: 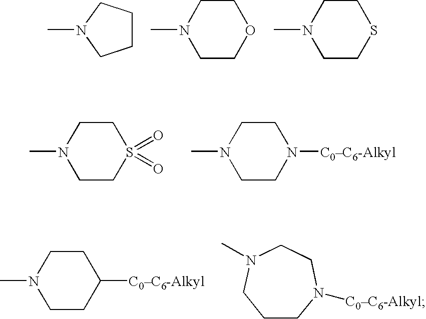
   or
   independently of one another hydroxy-C₁-C₆-alkylene, hydroxy-C₃-C₆-alkenylene, hydroxy-C₃-C₆-alkynylene, C₁-C₆-alkyloxy, C₃-C₇-cycloalkyloxy, C₃-C₇-Cycloalkyl-C₁-C₆-alkylenoxy, C₁-C₆-alkyloxy-C₁-C₆-alkylene, C₃-C₇-cycloalkyloxy-C₁-C₆-alkylene, C₁-C₆-alkyloxy-C₃-C₆-alkenylene, C₁-C₆-alkyloxy-C₃-C₆-alkynylene, C₁-C₆-alkyloxyphenyl-C₁-C₆-alkylene, phenyloxy-C₁-C₆-alkylene, C₁-C₆-alkylamino, di(C₁-C₆-alkyl)amino, C₁-C₆-alkylamino-C₁-C₆-alkylene, di(C₁-C₆)-alkylamino-C₁-C₆-alkylene, C₃-C₇-Cycloalkyl-(C₀-C₆-alkyl)amino, C₁-C₆-acyl-(C₀-C₆-alkyl)amido, C₁-C₆-alkylaminocarbonyl, di(C₁-C₆-alkyl)aminocarbonyl, (C₃-C₇-cycloalkyl)aminocarbonyl, di(C₃-C₇-cycloalkyl)aminocarbonyl, C₃-C₇-cycloalkyl-C₁-C₆-alkyleneaminocarbonyl, C₁-C₆-alkylcarbonyl, C₃-C₇-cycloalkylcarbonyl, carboxy, carboxamido [—C(O)NH₂], C₁-C₆-alkyloxycarbonyl, C₁-C₃-alkylsulphanyl, C₁-C₆-alkysulphonyl, C₃-C₇-cycloalkylsulphonyl, C₃-C₇-cycloalkyl-C₁-C₆-alkylenesulphonyl, C₁-C₆-alkylaminosulphonyl, di(C₁₋₆-alkyl)aminosulphonyl, (C₃-C₇-cycloalkyl)aminosulphonyl, di(C₃-C₇-cycloalkyl)aminosulphonyl, C₃-C₇-cycloalkyl-C₁-C₆-alkyleneaminosulphonyl, C₁-C₆-alkylsulphonylamido, —N(C₀-C₆-alkyl)-C(O)—C₁-C₆-alkyl, —N(C₀-C₆-alkyl)-C(O)—C₃-C₇-cycloalkyl, —N(C₀-C₆-alkyl)-C(O)—N-di(C₀-C₆-alkyl), —N(C₀-C₆-alkyl)-C(O)—O—(C₀-C₆)alkyl, —N(C₀-C₆-alkyl)-C(O)—NH—C₃-C₇-cycloalkyl, —N(C₀-C₆-alkyl)-SO₂—C₁-C₆-alkyl, —N(C₀-C₆-alkyl)-SO₂—C₃-C₇-cycloalkyl, —N(C₀-C₆-alkyl)-SO₂—N-di(C₀-C₆-alkyl), —N(C₀-C₆-alkyl)-SO₂—NH—(C₃-C₇)-cycloalkyl, —C(O)—N(H)—C₂-C₆-alkylene-(C₁₋₆-alkyl)amine, —C(O)—N(H)—C₂-C₆-alkylene-[di(C₁-C₆-alkyl)]amine, —C(O)—N(H)—C₂-C₆-alkylene-(C₃-C₇-cycloalkyl)amine, —C(O)—N(H)—C₂-C₆-alkylene-(C₃-C₇-cycloalkyl-C₁-C₆-alkyl)amine, —S(O₂)—N(H)—C₂-C₆-alkylene-(C₁-C₆-alkyl)amine, —S(O₂)—N(H)—C₂-C₆-alkylene-[di(C₁-C₆-alkyl)]amine, —S(O₂)—N(H)—C₂-C₆-alkylene-(C₃-C₇-cycloalkyl)amine, S(O₂)—N(H)—C₂-C₆-alkylene-(C₃-C₇-cycloalkyl-C₁-C₆-alkylene)amine, —O—C₂-C₆-alkylene-(C₁-C₆-alkyl)amine, —O—C₂-C₆-alkylene-[di(C₁-C₆-alkylene)]amine,
   or the radicals: 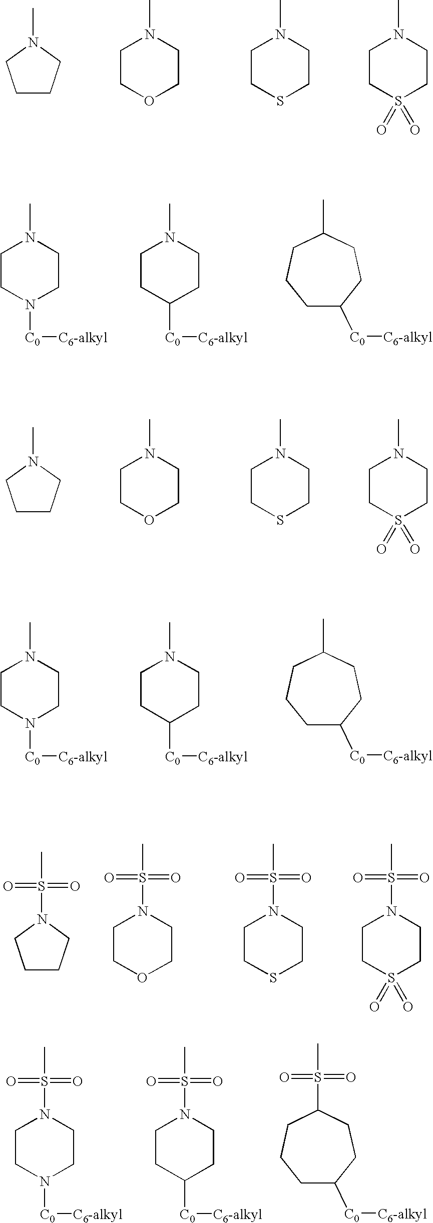 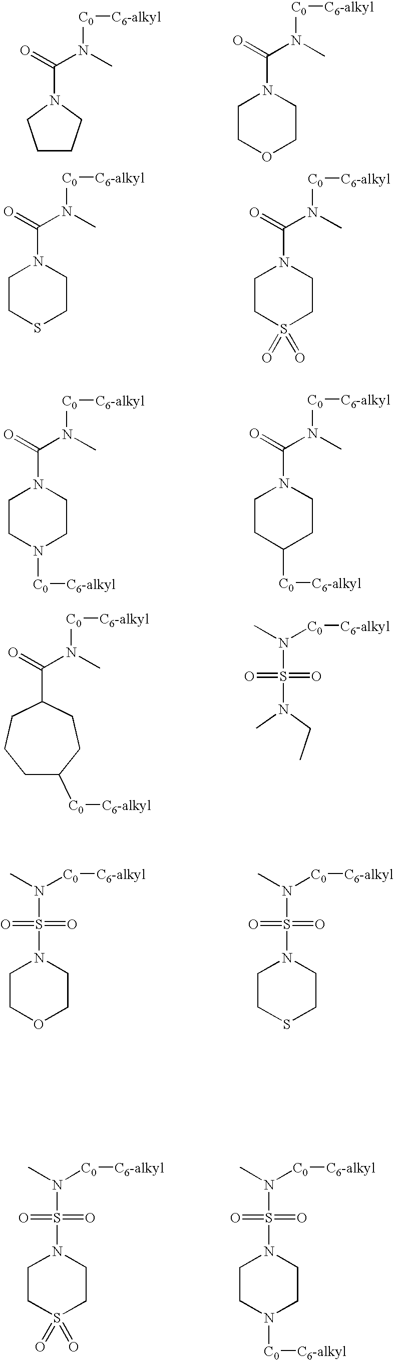 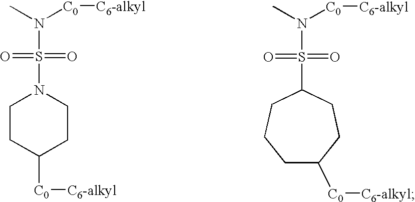
R7, R8 may be independently of one another hydrogen, methyl, ethyl, where the methyl and ethyl radicals may be fluorinated one or more times;
   where
R2 may substitute one or more positions of the aryl or heteroaryl ring in the indole residue;
R3 may substitute one or more positions of the aryl or heteroaryl ring in the radical Q;
R5 and R6 may together form heterocycloalkyl, cycloalkyl;
Q and W may be independently of one another aryl, heteroaryl;
X may be a bond, C₁-C₄-alkylene, C₂-C₄-alkenylene, C₂-C₄-alkynylene, C₁-C₃-alkyleneoxy, C₁-C₃-alkyleneoxy-C₁-C₃-alkylene,
Y may be a bond, C₁-C₄-alkylene.

The object has likewise been achieved according to the present invention by the compounds of the formula I in which R7 and R8 are a hydrogen, that is to say by the compounds of the formula Ia 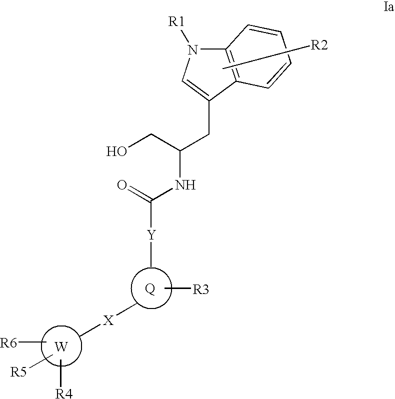
where
R1 may be hydrogen, C₁-C₆-alkyl, C₃-C₆-alkenyl or C₃-C₆-alkynyl, where the hydrocarbon radicals therein may optionally be substituted one or more times by fluorine;
R2 may be hydrogen, halogen, C₁-C₆-alkyl, C₂-C₆-alkenyl or C₂-C₆-alkynyl, C₁-C₄-alkyloxy, where the hydrocarbon chain therein may optionally be substituted one or more times by fluorine; or benzyloxy;
R3 may be hydrogen, halogen, nitro, amino, cyano, C₁-C₆-alkyl, C₂-C₆-alkenyl or C₂-C₆-alkynyl, C₁-C₄-alkykoxy, where the hydrocarbon chain therein may optionally be substituted one or more times by fluorine;
R4, R5, R6 may be independently of one another hydrogen, halogen, C₁-C₆-alkyl, C₂-C₆-alkenyl, C₂-C₆-alkynyl, C₁-C₄-alkyloxy, where the hydrocarbon chain therein may optionally be substituted one or more times by fluorine, C₁-C₃-alkylsulphanyl, acetamido, C₁-C₆-alkylaminocarbonyl; hydroxy, cyano, hydroxy-C₁₋₄-alkyl;
   where
R2 and R3 may substitute one or more positions of the aryl or heteroaryl ring in each case in the radical Q and in the indole residue;
R5 and R6 may together form heterocycloalkyl, cycloalkyl;
Q and W may be independently of one another aryl, heteroaryl;
X may be a bond, C₁-C₄-alkylene, C₁-C₄-alkenylene, C₁-C₄-alkynylene, C₁-C₃-alkyleneoxy, C₁-C₃-alkyleneoxy-C₁-C₃-alkylene,
Y may be a bond, C₁-C₄-alkylene.

The present invention relates to both possible enantiomeric forms at the stereocentre of the tryptophanol residue.

The unbranched C₁-C₆-alkyl groups for the radicals R1 to R6 may be for example a methyl, ethyl, propyl, butyl, pentyl or a hexyl group; and the branched C₃-C₆-alkyl groups for the radicals R1 to R6 may be an isopropyl, isobutyl, sec-butyl, tert-butyl, isopentyl, 2-methylbutyl, 1-methylbutyl, 1-ethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl or a 1,2-dimethylbutyl group.

The branched or unbranched C₃-C₆-alkenyl groups for the radical R1 may be for example an allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl- or a 1-(1,1-dimethylethyl)ethenyl group.

The C₃-C₆-alkynyl groups for the radical R1 may be for example a prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or a 3,3-dimethylbut-1-ynyl group.

The C₂-C₆-alkenyl groups for the radicals R2 to R6 may, in addition to the C₃-C₆-alkenyl groups mentioned for the radical R1, be for example a vinyl group.

The C₂-C₆-alkynyl groups for the radicals R2 to R6 may, in addition to the C₃-C₆-alkynyl groups mentioned for the radical R1, be for example an ethynyl group.

The C₁-C₆-alkyloxy groups for the radicals R2 to R6 may be for example a methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)oxy, (1,3-dimethylbutyl)oxy or a (1,2-dimethylbutyl)oxy group.

The halogens for the radicals R2 to R6 are fluorine, chlorine, bromine or iodine.

The C₁-C₃-alkylsulphanyl groups for the radicals R4 to R6 may be for example a methylsulphanyl (CH₃S—), ethylsulphanyl (CH₃CH₂S—), propylsulphanyl, isopropylsulphanyl group.

The C₁-C₆-alkylaminocarbonyl groups for the radicals R4 to R6 may be for example a methylaminocarbonyl-, ethylaminocarbonyl-, propylaminocarbonyl-, isopropylaminocarbonyl-, butylaminocarbonyl-, isobutylaminocarbonyl-, sec-butylaminocarbonyl-, tert-butylaminocarbonyl-, pentylaminocarbonyl-, isopentylaminocarbonyl-, (2-methylbutyl)aminocarbonyl-, (1-methylbutyl)aminocarbonyl-, (1-ethylpropyl)aminocarbonyl-, neopentylaminocarbonyl-, (1,1-dimethylpropyl)aminocarbonyl-, hexylaminocarbonyl-, (4-methylpentyl)aminocarbonyl-, (3-methylpentyl)aminocarbonyl-, (2-methylpentyl)aminocarbonyl-, (1-methylpentyl)aminocarbonyl-, (1-ethylbutyl)aminocarbonyl-, (2-ethylbutyl)aminocarbonyl-, (3,3-dimethylbutyl)aminocarbonyl-, (2,2-dimethylbutyl)aminocarbonyl-, (1,1-dimethylbutyl)aminocarbonyl-, (2,3-dimethylbutyl)aminocarbonyl-, (1,3-dimethylbutyl)aminocarbonyl- or a (1,2-dimethylbutyl)aminocarbonyl group.

The hydroxy-C₁-C₆-alkylene groups for the radicals R3 to R6 may be a hydroxymethyl (HOCH₂—), 2-hydroxyethyl (HOCH₂CH₂—), 1-hydroxyethyl [CH₃CH(OH)—], 3-hydroxypropyl (HOCH₂CH₂CH₂—), 2-hydroxypropyl [CH₃CH(OH)CH₂—], 1-hydroxypropyl [CH₃CH₂CH(OH)—], 2-hydroxy-1-methylethyl [HOCH₂CH(CH₃)—], 1-hydroxy-1-methylethyl [(CH₃)₂C(OH)—], 4-hydroxybutyl (HOCH₂CH₂CH₂CH₂—), 3-hydroxybutyl [CH₃CH(OH)CH₂CH₂—], 2-hydroxybutyl [CH₃CH₂CH(OH)CH₂—], 1-hydroxybutyl [CH₃CH₂CH₂CH(OH)—], 3-hydroxy-1-methylpropyl [HOCH₂CH₂CH(CH₃)—], 2-hydroxy-1-methylpropyl [CH₃CH(OH)CH(CH₃)—], 1-hydroxy-1-methylpropyl [CH₃CH₂C(CH₃)(OH)—], 1-(hydroxymethyl)propyl [CH₃CH(CH₂OH)—], 3-hydroxy-2-methylpropyl [HOCH₂CH(CH₃)CH₂—], 2-hydroxy-2-methylpropyl [(CH₃)₂C(OH)CH₂—], 1-hydroxy-2-methylpropyl [CH₃CH(CH₃)CH(OH)—] or a 2-hydroxy-1,1-dimethylethyl group [HOCH₂C(CH₃)₂—].

The heterocycloalkyl groups which may form the radicals R5 and R6 together may be for example the following groups:

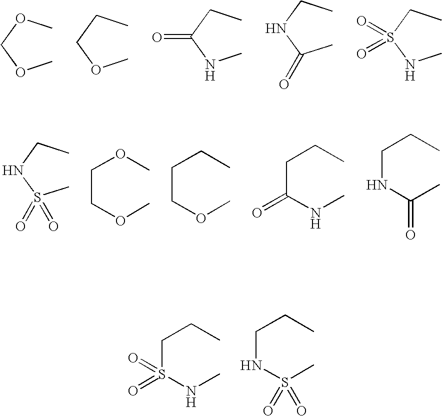

The cycloalkyl groups which may form the radicals R5 and R6 together may be for example the following groups:

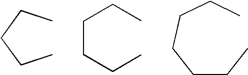

The C₃-C₇-cycloalkyl groups for the radicals R1 to R6 may be for example a cyclopropyl, cyclobutyl, cyclopentyl, cyclohexyl, cycloheptyl group.

The C₃-C₇-heterocycloalkyl groups for the radicals R1 to R6 may be for example a cyclopropyl, cyclobutyl, cycopentyl, cyclohexyl, cycloheptyl group in which one or two carbon atoms of the ring are replaced independently of one another by an oxygen, nitrogen or sulphur atom.

The aryl groups for the radicals Q and W may be for example a phenyl, naphthyl group which is linked via substitutable positions.

The heteroaryl groups for the radicals Q and W may be for example a pyridinyl, pyrimidinyl, quinolinyl, isoquinolinyl, quinazolinyl, quinoxalinyl, phthalazinyl, 1,5-naphthyridinyl, 1,6-naphthyridinyl, 1,7-naphthyridinyl, 1,8-naphthyridinyl, benzofuranyl, benzothienyl, 1,3-benzodioxolyl, 2,1,3-benzothiadiazolyl, indolyl, furanyl, thienyl, oxazolyl, isoxazolyl, thiazolyl, pyrrolyl, pyrazolyl or an imidazolyl group which is linked via substitutable positions.

The C₁-C₄-alkylene groups for the radicals X and Y may be for example a methylene (—CH₂—), ethylidene [—CH(CH₃)—], ethylene (—CH₂CH₂—), prop-1,3-ylene (—CH₂CH₂CH₂—), prop-1,2-ylene [—CH₂CH(CH₃)—], but-1,4-ylene (—CH₂CH₂CH₂CH₂—), but-1,3-ylene [—CH₂CH₂CH(CH₃)—], but-1,2-ylene [—CH₂CH(CH₂CH₃)—], but-2,3-ylene [—CHCH(CH₃)—], 2-methylprop-1,2-ylene [—CH₂C(CH₃)₂—] or a 2-methylprop-1,3-ylene group [—CH₂CH(CH₃)CH₂—].

The C₂-C₄-alkenylene groups for the radical X may be for example an ethen-1,2-ylidene (—CH═CH—), prop-2-en-1,3-ylidene (—CH₂—CH═CH—), prop-1-en-1,3-ylidene (—CH═CH—CH₂—), but-1-en-1,4-ylidene (—CH═CH—CH₂—CH₂—), but-2-en-1,4-ylidene (—CH₂—CH═CH—CH₂—) or a but-3-en-1,4-ylidene group (—CH₂—CH₂—CH═CH—).

The C₂-C₄-alkynylene groups for the radical X may be for example an ethyn-1,2-ylidene (—C≡C—), prop-2-yn-1,3-ylidene (—CH₂—C≡C—), prop-1-yn-1,3-ylidene (—C≡C—CH₂—), but-1-yn-1,4-ylidene (—C≡C—CH₂—CH₂—), but-2-yn-1,4-ylidene (—CH₂—C≡C—CH₂—) or a but-3-yn-1,4-ylidene group (—CH₂—CH₂—C≡C—).

The C₁-C₃-alkyleneoxy groups for the radical X may be for example an oxymethylene (—O—CH₂—), methyleneoxy (—CH₂—O—), ethane-1,2-diyloxy (—CH₂—CH₂—O—), oxyethane-1,2-diyl (—O—CH₂—CH₂—), propane-1,3-diyloxy (—CH₂—CH₂—CH₂—O—) or an oxypropane-1,3-diyl (—O—CH₂—CH₂—CH₂—) group.

The C₁-C₃-alkyleneoxy-C₁-C₃-alkyl groups for the radical X may be for example an oxybis(methylene) (—CH₂—O—CH₂—), methyleneoxyethane-2,1-diyl [—CH₂—O—(CH₂)₂—], ethane-1,2-diyloxymethylene [—(CH₂)₂—O—CH₂—], methyleneoxypropane-3,1-diyl [—CH₂—O—(CH₂)₃—], propane-1,3-diyloxymethylene [—(CH₂)₃—O—CH₂—], oxybis(ethane-2,1-diyl) [—(CH₂)₂—O—(CH₂)₂—], propane-1,3-diyloxyethane-2,1-diyl [—(CH₂)₃—O—(CH₂)₂—], ethane-1,2-diyloxypropane-3,1-diyl [—(CH₂)₂—O—(CH₂)₃—] or an oxybis(propane-3,1-diyl) group [—(CH₂)₃—O—(CH₂)₃—].

The C₃-C₇-cycloalkyloxy groups for the radicals R1 to R6 may be for example a cyclopropyloxy, cyclobutyloxy, cyclopentyloxy, cyclohexyloxy, cycloheptyloxy group.

The C₁-C₆-alkylamino groups for the radicals R1 to R6 may be for example methylamino, ethylamino, propylamino, isopropylamino, butylamino, isobutylamino, sec-butylamino, tert-butylamino, pentylamino, isopentylamino, (2-methylbutyl)amino, (1-methylbutyl)amino, (1-ethylpropyl)amino, neopentylamino, (1,1-dimethylpropyl)amino, hexylamino, (4-methylpentyl)amino, (3-methylpentyl)amino, (2-methylpentyl)amino, (1-methylpentyl)amino, (1-ethylbutyl)amino, (2-ethylbutyl)amino, (3,3-dimethylbutyl)amino, (2,2-dimethylbutyl)amino, (1,1-dimethylbutyl)amino, (2,3-dimethylbutyl)amino, (1,3-dimethylbutyl)amino or a (1,2-dimethylbutyl)amino group.

In the di(C₁-C₆-alkyl)amino groups for the radicals R1 to R6, each of the two radicals on the nitrogen atom of the dialkylamino group may be chosen independently of one another from the following radicals: possible examples are a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the C₃-C₇-cycloalkyl-C₁-C₆-alkyleneoxy groups for the radicals R1 to R6 it is possible to combine each of the C₃-C₇-cycloalkyl groups of the C₃-C₇-cycloalkyl-C₁-C₆-alkyleneoxy group, for example of a cyclopropyl, cyclobutyl, cyclopentyl, cyclohexyl or cycloheptyl group, independently of one another with each C₀-C₆-alkyleneoxy group, for example with a methyleneoxy, ethyleneoxy, propyleneoxy, butyleneoxy, pentyleneoxy, hexyleneoxy group.

In the hydroxy-C₃-C₆-alkenylene groups for the radicals R1 to R6 it is possible for the hydroxy group to be located on any desired position of the C₃-C₆-alkenyl group, for example of an allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-Pent-2-enyl-, (Z)-Pent-2-enyl-, (E)-Pent-1-enyl-, (Z)-Pent-1-enyl-, hex-5-enyl-, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl or a 1-(1,1-dimethylethyl)ethenyl group, and to be combined independently of one another.

In the hydroxy-C₃-C₆-alkynyl groups for the radicals R1 to R6 it is possible for the hydroxy group to be located at any desired position of the C₃-C₆-alkynyl group, for example of a prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or a 3,3-dimethylbut-1-ynyl group.

In the C₁-C₆-alkyloxy-C₃-C₆-alkenylene groups for the radicals R1 to R6 it is possible for the C₁-C₆-alkyloxy group, for example a methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)oxy, (1,3-dimethylbutyl)oxy or a (1,2-dimethylbutyl)oxy group, to be located on any desired position of the C₃-C₆-alkenyl group, for example of an allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl or a 1-(1,1-dimethylethyl)ethenyl group and to be combined independently of one another.

In the C₁-C₆-alkyloxy-C₃-C₆-alkynylene groups for the radicals R1 to R6 it is possible for the C₁-C₆-alkyloxy group, for example a methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)oxy, (1,3-dimethylbutyl)oxy or a (1,2-dimethylbutyl)oxy group, to be located at any desired position of the C3-C6-alkynyl group, for example of a prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or a 3,3-dimethylbut-1-ynyl group, and to be combined independently of one another.

In the C₁-C₆-alkyloxyphenyl-C₁-C₆-alkylene groups for the radical R1 to R6 it is possible for the C₁-C₆-alkyloxy group to be selected independently of one another from methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)oxy, (1,3-dimethylbutyl)oxy or a (1,2-dimethylbutyl)oxy, and to be combined independently of one another with C₁-C₆-alkylene groups such as, for example, methylene, ethylene, propylene, butylene, pentylene, hexylene.

In the C₃-C₇-cycloalkyl-(C₀-C₆)-alkyleneamino groups of the radicals R3 to R6 it is possible for each of the C₃-C₇-cycloalkyl groups of the C₃-C₇-cycloalkyl-(C₀-C₆)-alkyleneamino group, for example of a cyclopropyl, cyclobutyl, cyclopentyl, cyclohexyl or cycloheptyl group, to be combined independently of one another with each C₀-C₆-alkylene group, for example with a bond, a methylene, ethylene, propylene, butylene, pentylene, hexylene group.

In the C₁-C₆-alkyloxy-C₁-C₆-alkylene groups for the radical R1 to R6, it is possible for the C₁-C₆-alkyloxy group to be selected independently for example from methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)oxy, (1,3-dimethylbutyl)oxy or a (1,2-dimethylbutyl)oxy and to be combined independently of one another with C₁-C₆-alkylene groups such as, for example, methylene, ethylene, propylene, butylene, pentylene, hexylene.

In the di(C₁-C₆-alkyl)amino-C₁-C₆-alkylene group for the radical R1 it is possible for each of the two radicals on the nitrogen atom of the amino group to be selected independently for example from methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, and to be combined with C₁-C₆-alkylene groups such as, for example, methylene, ethylene, propylene, butylene, pentylene, hexylene.

The C₃-C₇-cycloalkyl-C₁-C₆-alkylene groups for the radicals R1 to R6 may be for example a cyclopropyloxymethylene, cyclopropyloxyethylene, cyclopropyloxypropylene, cyclopropyloxybutylene, cyclopropyloxypentylene, cyclopropyloxyhexylene, cyclobutyloxymethylene, cyclobutyloxyethylene, cyclobutyloxypropylene, cyclobutyloxybutylene, cyclobutyloxypentylene, cyclobutyloxyhexylene, cyclopentyloxymethylene, cyclopentyloxyethylene, cyclopentyloxypropylene, cyclopentyloxybutylene, cyclopentyloxypentylene, cyclopentyloxyhexylene, cyclohexyloxymethylene, cyclohexyloxyethylene, cyclohexyloxypropylene, cyclohexyloxybutylene, cyclohexyloxypentylene, cyclohexyloxyhexylene, cycloheptyloxymethylene, cycloheptyloxyethylene, cycloheptyloxypropylene, cycloheptyloxybutylene, cycloheptyloxypentylene, cycloheptyloxyhexylen group.

In the C₁-C₆-alkylamino-C₁-C₆-alkylene groups for the radicals R1 to R6 it is possible for the C₁-C₆-alkylamino group to be selected independently for example from methylamino, ethylamino, propylamino, isopropylamino, butylamino, isobutylamino, sec-butylamino, tert-butylamino, pentylamino, isopentylamino, (2-methylbutyl)amino, (1-methylbutyl)amino, (1-ethylpropyl)amino, neopentylamino, (1,1-dimethylpropyl)amino, hexylamino, (4-methylpentyl)amino, (3-methylpentyl)amino, (2-methylpentyl)amino, (1-methylpentyl)amino, (1-ethylbutyl)amino, (2-ethylbutyl)amino, (3,3-dimethylbutyl)amino, (2,2-dimethylbutyl)amino, (1,1-dimethylbutyl)amino, (2,3-dimethylbutyl)amino, (1,3-dimethylbutyl)amino or a (1,2-dimethylbutyl)amino and to be combined with C₁-C₆-alkylene groups such as, for example, methylene, ethylene, propylene, butylene, pentylene, hexylene.

The phenyloxy-C₁-C₆-alkylene groups for the radicals R1 to R6 may be for example a phenyloxymethyl, phenyloxyethyl, phenyloxypropyl, phenyloxybutyl, phenyloxypentyl, phenyloxyhexyl group.

In the C₁-C₆-acyl-(C₀-C₆-alkyl)amido groups for the radicals R4 to R6, it is possible for each of the C₁-C₆-acyl groups, for example a formyl, acetyl, propionyl, 2-methylpropionyl, 2,2-dimethylpropionyl, butyryl, 2-methylbutyryl, 3-methylbutyryl, 2,2-dimethylbutyryl, 2-ethylbutyryl, pentanoyl, 2-methylpentanoyl, 3-methylpentanoyl, 4-methylpentanoyl or a hexanoyl group, to be combined independently of one another with each (C₀-C₆-alkyl)amido group, for example a hydrogen atom, a methylamido, ethylamido, propylamido, isopropylamido, butylamido, isobutylamido, sec-butylamido, tert-butylamido, pentylamido, isopentylamido, (2-methylbutyl)amido, (1-methylbutyl)amido, (1-ethylpropyl)amido, neopentylamido, (1,1-dimethylpropyl)amido, hexylamido, (4-methylpentyl)amido, (3-methylpentyl)amido, (2-methylpentyl)amido, (1-methylpentyl)amido, (1-ethylbutyl)amido, (2-ethylbutyl)amido, (3,3-dimethylbutyl)amido, (2,2-dimethylbutyl)amido, (1,1-dimethylbutyl)amido, (2,3-dimethylbutyl)amido, (1,3-dimethylbutyl)amido or a (1,2-dimethylbutyl)amido group.

The C₁-C₆-alkylaminocarbonyl groups for the radicals R4 to R6 may be for example a methylaminocarbonyl, ethylaminocarbonyl, propylaminocarbonyl, isopropylaminocarbonyl, butylaminocarbonyl, isobutylaminocarbonyl, sec-butylaminocarbonyl, tert-butylaminocarbonyl, pentylaminocarbonyl, isopentylaminocarbonyl, (2-methylbutyl)aminocarbonyl, (1-methylbutyl)aminocarbonyl, (1-ethylpropyl)aminocarbonyl, neopentylaminocarbonyl, (1,1-dimethylpropyl)aminocarbonyl, hexylaminocarbonyl, (4-methylpentyl)aminocarbonyl, (3-methylpentyl)aminocarbonyl, (2-methylpentyl)aminocarbonyl, (1-methylpentyl)aminocarbonyl, (1-ethylbutyl)aminocarbonyl, (2-ethylbutyl)aminocarbonyl, (3,3-dimethylbutyl)aminocarbonyl, (2,2-dimethylbutyl)aminocarbonyl, (1,1-dimethylbutyl)aminocarbonyl, (2,3-dimethylbutyl)aminocarbonyl, (1,3-dimethylbutyl)aminocarbonyl or a (1,2-dimethylbutyl)aminocarbonyl group.

In the di(C₁-C₆-alkyl)aminocarbonyl groups for the radicals R4 to R6, each of the two C₁-C₆-alkyl radicals on the nitrogen atom of the di(C₁-C₆-alkyl)aminocarbonyl group may be independently of one another for example a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

The (C₃-C₇-cycloalkyl)aminocarbonyl groups for the radicals R4 to R6 may be for example a cyclopropylaminocarbonyl, cyclobutylaminocarbonyl, cyclopentylaminocarbonyl, cyclohexylaminocarbonyl or cycloheptylaminocarbonyl group.

In the di(C₃-C₇-cycloalkyl)aminocarbonyl groups for the radicals R4 to R6, each of the two C₃-C₇-cycloalkyl radicals on the nitrogen atom of the di(C₃-C₇-cycloalkyl)aminocarbonyl group may be independently of one another for example a cyclopropyl, cyclobutyl, cyclopentyl, cyclohexyl or cycloheptyl group.

In the C₃-C₇-cycloalkyl-C₁-C₆-alkyleneaminocarbonyl groups of the radicals R4 to R6 it is possible for each of the C₃-C₇-cycloalkyl groups of the C₃-C₇-cycloalkyl-C₁-C₆-alkyleneaminocarbonyl groups, for example of a cyclopropyl, cyclobutyl, cyclopentyl, cyclohexyl or cycloheptyl group, to be combined independently of one another with each C₁-C₆-alkyleneaminocarbonyl group, for example with a methyleneaminocarbonyl, ethyleneaminocarbonyl, propyleneaminocarbonyl, butyleneaminocarbonyl, pentyleneaminocarbonyl, hexyleneaminocarbonyl group.

The C₁-C₆-alkylcarbonyl groups for the radicals R4 to R6 may be for example a methylcarbonyl, ethylcarbonyl, propylcarbonyl, isopropylcarbonyl, butylcarbonyl, isobutylcarbonyl, sec-butylcarbonyl, tert-butylcarbonyl, pentylcarbonyl, isopentylcarbonyl, (2-methylbutyl)carbonyl, (1-methylbutyl)carbonyl, (1-ethylpropyl)carbonyl, neopentylcarbonyl, (1,1-dimethylpropyl)carbonyl, hexylcarbonyl, (4-methylpentyl)carbonyl, (3-methylpentyl)carbonyl, (2-methylpentyl)carbonyl, (1-methylpentyl)carbonyl, (1-ethylbutyl)carbonyl, (2-ethylbutyl)carbonyl, (3,3-dimethylbutyl)carbonyl, (2,2-dimethylbutyl)carbonyl, (1,1-dimethylbutyl)carbonyl, (2,3-dimethylbutyl)carbonyl, (1,3-dimethylbutyl)carbonyl or a (1,2-dimethylbutyl)carbonyl group.

The C₃-C₇-cycloalkylcarbonyl groups for the radicals R4 to R6 may be for example a cyclopropylcarbonyl, cyclobutylcarbonyl, cyclopentylcarbonyl, cyclohexylcarbonyl or cycloheptylcarbonyl group.

The C₁-C₆-alkyloxycarbonyl groups for the radicals R4 to R6 may be for example a methyloxycarbonyl, ethyloxycarbonyl, propyloxycarbonyl, isopropyloxycarbonyl, butyloxycarbonyl, isobutyloxycarbonyl, sec-butyloxycarbonyl, tert-butyloxycarbonyl, pentyloxycarbonyl, isopentyloxycarbonyl, (2-methylbutyl)oxycarbonyl, (1-methylbutyl)oxycarbonyl, (1-ethylpropyl)oxycarbonyl, neopentyloxycarbonyl, (1,1-dimethylpropyl)oxycarbonyl, hexyloxycarbonyl, (4-methylpentyl)oxycarbonyl, (3-methylpentyl)oxycarbonyl, (2-methylpentyl)oxycarbonyl, (1-methylpentyl)oxycarbonyl, (1-ethylbutyl)oxycarbonyl, (2-ethylbutyl)oxycarbonyl, (3,3-dimethylbutyl)oxycarbonyl, (2,2-dimethylbutyl)oxycarbonyl, (1,1-dimethylbutyl)oxycarbonyl, (2,3-dimethylbutyl)oxycarbonyl, (1,3-dimethylbutyl)oxycarbonyl or a (1,2-dimethylbutyl)oxycarbonyl group.

The C₁-C₆-alkylsulphonyl groups for the radicals R4 to R6 may be for example a methylsulphonyl, ethylsulphonyl, propylsulphonyl, isopropylsulphonyl, butylsulphonyl, isobutylsulphonyl, sec-butylsulphonyl, tert-butylsulphonyl, pentylsulphonyl, isopentylsulphonyl, (2-methylbutyl)sulphonyl, (1-methylbutyl)sulphonyl, (1-ethylpropyl)sulphonyl, neopentylsulphonyl, (1,1-dimethylpropyl)sulphonyl, hexylsulphonyl, (4-methylpentyl)sulphonyl, (3-methylpentyl)sulphonyl, (2-methylpentyl)sulphonyl, (1-methylpentyl)sulphonyl, (1-ethylbutyl)sulphonyl, (2-ethylbutyl)sulphonyl, (3,3-dimethylbutyl)sulphonyl, (2,2-dimethylbutyl)sulphonyl, (1,1-dimethylbutyl)sulphonyl, (2,3-dimethylbutyl)sulphonyl, (1,3-dimethylbutyl)sulphonyl or a (1,2-dimethylbutyl)sulphonyl group.

The C₃-C₇-cycloalkylsulphonyl groups for the radicals R4 to R6 may be for example a cyclopropylsulphonyl, cyclobutylsulphonyl, cyclopentylsulphonyl, cyclohexylsulphonyl or cycloheptylsulphonyl group.

In the C₃-C₇-cycloalkyl-C₁-C₆-alkylenesulphonyl groups of the radicals R4 to R6 it is possible for each of the C₃-C₇-cycloalkyl groups of the C₃-C₇-cycloalkyl-C₁-C₆-alkylenesulphonyl groups, for example of a cyclopropyl, cyclobutyl, cyclopentyl, cyclohexyl or cycloheptyl group, to be combined independently of one another with each C₁-C₆-alkylenesulphonyl group, for example with a methylenesulphonyl, ethylenesulphonyl, propylenesulphonyl, butylenesulphonyl, pentylenesulphonyl, hexylenesulphonyl group.

The C₁-C₆-alkylaminosulphonyl groups for the radicals R4 to R6 may be for example a methylaminosulphonyl, ethylaminosulphonyl, propylaminosulphonyl, isopropylaminosulphonyl, butylaminosulphonyl, isobutylaminosulphonyl, sec-butylaminosulphonyl, tert-butylaminosulphonyl, pentylaminosulphonyl, isopentylaminosulphonyl, (2-methylbutyl)aminosulphonyl, (1-methylbutyl)aminosulphonyl, (1-ethylpropyl)aminosulphonyl, neopentylaminosulphonyl, (1,1-dimethylpropyl)aminosulphonyl, hexylaminosulphonyl, (4-methylpentyl)aminosulphonyl, (3-methylpentyl)aminosulphonyl, (2-methylpentyl)aminosulphonyl, (1-methylpentyl)aminosulphonyl, (1-ethylbutyl)aminosulphonyl, (2-ethylbutyl)aminosulphonyl, (3,3-dimethylbutyl)aminosulphonyl, (2,2-dimethylbutyl)aminosulphonyl, (1,1-dimethylbutyl)aminosulphonyl, (2,3-dimethylbutyl)aminosulphonyl, (1,3-dimethylbutyl)aminosulphonyl or a (1,2-dimethylbutyl)aminosulphonyl group.

In the di(C₁-C₆-alkyl)aminosulphonyl groups for the radicals R4 to R6, each of the two C₁-C₆-alkyl radicals on the nitrogen atom of the di(C₁-C₆-alkyl)aminosulphonyl group may be independently of one another for example a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

The (C₃-C₇-cycloalkyl)aminosulphonyl groups for the radicals R4 to R6 may be for example a cyclopropylaminosulphonyl, cyclobutylaminosulphonyl, cyclopentylaminosulphonyl, cyclohexylaminosulphonyl or cycloheptylaminosulphonyl group.

In the di(C₃-C₇-cycloalkyl)aminosulphonyl groups for the radicals R4 to R6, each of the two C₃-C₇-cycloalkyl radicals on the nitrogen atom of the di(C₃-C₇-cycloalkyl)aminosulphonyl group may be independently of one another for example a cyclopropyl, cyclobutyl, cyclopentyl, cyclohexyl or cycloheptyl group.

In the C₃-C₇-cycloalkyl-C₁-C₆-alkyleneaminosulphonyl groups of the radicals R4 to R6, each of the C₃-C₇-cycloalkyl groups of the C₃-C₇-cycloalkyl-C₁-C₆-alkyleneaminosulphonyl groups, for example of a cyclopropyl, cyclobutyl, cyclopentyl, cyclohexyl or cycloheptyl group, can be combined independently of one another with each C₁-C₆-alkyleneaminosulphonyl group, for example with a methyleneaminosulphonyl, ethyleneaminosulphonyl, propyleneaminosulphonyl, butyleneaminosulphonyl, pentyleneaminosulphonyl, hexyleneaminosulphonyl group.

The C₁-C₆-alkylsulphonylamido groups for the radicals R4 to R6 may be for example a methylsulphonylamido, ethylsulphonylamido, propylsulphonylamido, isopropylsulphonylamido, butylsulphonylamido, isobutylsulphonylamido, sec-butylsulphonylamido, tert-butylsulphonylamido, pentylsulphonylamido, isopentylsulphonylamido, (2-methylbutyl)sulphonylamido, (1-methylbutyl)sulphonylamido, (1-ethylpropyl)sulphonylamido, neopentylsulphonylamido, (1,1-dimethylpropyl)sulphonylamido, hexylsulphonylamido, (4-methylpentyl)sulphonylamido, (3-methylpentyl)sulphonylamido, (2-methylpentyl)sulphonylamido, (1-methylpentyl)sulphonylamido, (1-ethylbutyl)sulphonylamido, (2-ethylbutyl)sulphonylamido, (3,3-dimethylbutyl)sulphonylamido, (2,2-dimethylbutyl)sulphonylamido, (1,1-dimethylbutyl)sulphonylamido, (2,3-dimethylbutyl)sulphonylamido, (1,3-dimethylbutyl)sulphonylamido or a (1,2-dimethylbutyl)sulphonylamido group.

In the —N(C₀-C₆-alkyl)-C(O)—C₁-C₆-alkyl groups of the radicals R4 to R6, each of the (C₀-C₆-alkyl) groups on the nitrogen atom of the —N(C₀-C₆-alkyl)-C(O)—C₁-C₆-alkyl groups, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may be combined independently of one another with each C₁-C₆-alkyl group on the carbonyl group of the amide, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N—(C₀-C₆-alkyl)-C(O)—C₃-C₇-cycloalkyl groups of the radicals R4 to R6, each of the (C₀-C₆-alkyl) groups on the nitrogen atom of the —N(C₀-C₆-alkyl)-C(O)—C₁-C₆-alkyl groups, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may be combined independently of one another with each C₃-C₇-cycloalkyl group on the carbonyl group of the amide, for example with a cyclopropyl, cyclobutyl, cyclopentyl, cyclohexyl or cycloheptyl group.

In the —N(C₀-C₆-alkyl)-C(O)—N-di(C₀-C₆-alkyl) groups of the radicals R4 to R6, all three (C₀-C₆-alkyl) groups may be independently of one another a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N(C₀-C₈-alkyl)-C(O)—O—(C₀-C₆-alkyl) groups of the radicals R4 to R6, both (C₀-C₆-alkyl) groups may be independently of one another a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N(C₀-C₆-alkyl)-C(O)—NH—(C₃-C₇-cycloalkyl) groups of the radicals R4 to R6, each of the (C₀-C₆-alkyl) groups on the nitrogen atom of the —N(C₀-C₆-alkyl)-C(O)—NH—(C₃-C₇-cycloalkyl) groups, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may independently of one another be combined with each C₃-C₇-cycloalkyl group on the terminal nitrogen atom of the urea, for example with a cyclopropyl, cyclobutyl, cyclopentyl, cyclohexyl or cycloheptyl group.

In the —N(C₀-C₆-alkyl)-SO₂—(C₁-C₆-alkyl) groups of the radicals R4 to R6, each of the (C₀-C₆-alkyl) groups on the nitrogen atom of the —N(C₀-C₆-alkyl)-SO₂—(C₁-C₆-alkyl) group, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may independently of one another be combined with each C₁-C₆-alkyl group on the sulphonyl group of the sulphonamide, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N(C₀-C₆-alkyl)-SO₂—C₃-C₇-cycloalkyl groups of the radicals R4 to R6, each of the (C₀-C₆-alkyl) groups on the nitrogen atom of the —N(C₀-C₆-alkyl)-SO₂—C₃-C₇-cycloalkyl group, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may be combined independently of one another with each C₃-C₇-cycloalkyl group on the sulphonyl group, for example with a cyclopropyl, cyclobutyl, cyclopentyl, cyclohexyl or cycloheptyl group.

In the —N(C₀-C₆-alkyl)-SO₂—N-di(C₀-C₆-alkyl) groups of the radicals R4 to R6, all three (C₀-C₆-alkyl) groups may be independently of one another a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N(C₀-C₆-alkyl)-SO₂—NH—(C₃-C₇)-cycloalkyl groups of the radicals R4 to R6, the C₀-C₆-alkyl group of the —N(C₀-C₆-alkyl)-SO₂—NH—(C₃-C₇)-cycloalkyl group, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may be combined independently of one another with each C₃-C₇-cycloalkyl group, for example with a cyclopropyl, cyclobutyl, cyclopentyl, cyclohexyl or cycloheptyl group.

In the —C(O)—N(H)—C₂-C₆-alkylene-(C₁-C₆-alkyl)amine groups of the radicals R4 to R6, each of the C₂-C₆-alkylene groups on the nitrogen atom of the —C(O)—N(H)—C₂-C₆-alkylene-(C₁-C₆-alkyl)amine group, for example an ethylene, propylene, butylene, pentylene or hexylene group, may be combined independently of one another with each C₁-C₆-alkyl group on the amino group, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —C(O)—N(H)—C₂-C₆-alkylene-[di(C₁-C₆-alkyl)]amine groups of the radicals R4 to R6, each of the C₂-C₆-alkylene groups on the nitrogen atom of the —C(O)—N(H)—C₂-C₆-alkylene-[di(C₁-C₆-alkyl)]amine group, for example an ethylene, propylene, butylene, pentylene or hexylene group, may be combined independently of one another with each of the two identically or different C₁-C₆-alkyl groups on the amino group, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl-, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —C(O)—N(H)—C₂-C₆-alkylene-(C₃-C₇-cycloalkyl)amine groups of the radicals R4 to R6, each of the (C₂-C₆-alkylene) groups of the —C(O)—N(H)—C₂-C₆-alkylene-(C₃-C₇-cycloalkyl)amine group, for example an ethylene, propylene, butylene, pentylene or hexylene group, may be combined independently of one another with each C₃-C₇-cycloalkyl group on the amine, for example with a cyclopropyl, cyclobutyl, cyclopentyl, cyclohexyl or cycloheptyl group.

In the —C(O)—N(H)—C₂-C₆-alkylene-(C₃-C₇-cycloalkyl-C₁-C₆-alkylene)amine groups of the radicals R4 to R6, each of the (C₂-C₆-alkylene) groups of the —C(O)—N(H)—C2-C6-alkylene-(C3-C6-cycloalkyl-C1-C6-alkylene)amine group, for example an ethylene, propylene, butylene, pentylene or hexylene group, may be combined independently of one another with each C₃-C₇-cycloalkyl-C₁-C₆-alkylene group on the amine, for example with a cyclopropylmethylene, cyclopropylethylene, cyclopropylpropylene, cyclopropylbutylene, cyclopropylpentylene, cyclopropylhexylene, cyclobutylmethylene, cyclobutylethylene, cyclobutylpropylene, cyclobutylbutylene, cyclobutylpentylene, cyclobutylhexylene, cyclopentylmethylene, cyclopentylethylene, cyclopentylpropylene, cyclopentylhexylene, cyclohexylmethylene, cyclohexylethylene, cyclohexylpropylene, cyclohexylbutylene, cyclohexylpentylene, cyclohexylhexylene, cycloheptylmethylene, cycloheptylethylene, cycloheptylpropylene, cycloheptylbutylene, cycloheptylpentylene or cycloheptylhexylene group.

In the —S(O₂)—N(H)—C₂-C₆-alkylene-(C₁-C₆-alkyl)amine groups of the radicals R4 to R6, the (C₂-C₆-alkylene) groups of the —S(O₂)—N(H)—C₂-C₆-alkylene-(C₁-C₆-alkyl)amine group, for example an ethylene, propylene, butylene, pentylene or hexylene group, may be combined independently of one another with each C₁-C₆-alkyl group on the amino group, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —S(O₂)—N(H)—C₂-C₆-alkylene-[di(C₁-C₆-alkyl)]amine groups of the radicals R4 to R6, the C₂-C₆-alkylene group of the —S(O₂)—N(H)—C₂-C₆-alkylene-[di(C₁-C₆-alkyl)]amine group, for example an ethylene, propylene, butylene, pentylene or hexylene group, may be combined independently of one another with each of the two C₁-C₆-alkyl groups on the amino group, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl-, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —S(O₂)—N(H)—C₂-C₆-alkylene-(C₃-C₇-cycloalkyl)amine groups of the radicals R4 to R6, the C₂-C₆-alkylene group of the —S(O₂)—N(H)—C₂-C₆-alkylene-(C₃-C₇-cycloalkyl)amine group, for example an ethylene, propylene, butylene, pentylene or hexylene group, may be combined independently of one another with each C₃-C₇-cycloalkyl group on the amino group, for example with a cyclopropyl, cyclobutyl, cyclopentyl, cyclohexyl or cycloheptyl group.

In the —S(O₂)—N(H)—C₂-C₆-alkylene-(C₃-C₇-cycloalkyl-C₁-C₆-alkylene)amine groups of the radicals R4 to R6, each C₂-C₆-alkylene group of the —S(O₂)—N(H)—C₂-C₆-alkylene-(C₃-C₇-cycloalkyl-C₁-C₆-alkylene)amine group, for example an ethylene, propylene, butylene, pentylene or hexylene group, may be combined independently of one another with each C₃-C₇-cycloalkyl-C₁-C₆-alkylene group on the amine, for example with a cyclopropylmethylene, cyclopropylethylene, cyclopropylpropylene, cyclopropylbutylene, cyclopropylpentylene, cyclopropylhexylene, cyclobutylmethylene, cyclobutylethylene, cyclobutylpropylene, cyclobutylbutylene, cyclobutylpentylene, cyclobutylhexylene, cyclopentylmethylene, cyclopentylethylene, cyclopentylpropylene, cyclopentylhexylene, cyclohexylmethylene, cyclohexylethylene, cyclohexylpropylene, cyclohexylbutylene, cyclohexylpentylene, cyclohexylhexylene, cycloheptylmethylene, cycloheptylethylene, cycloheptylpropylene, cycloheptylbutylene, cycloheptylpentylen or cycloheptylhexylene group.

In the —O—C₂-C₆-alkylene-(C₁-C₆-alkyl)amine groups of the radicals R4 to R6, the C₂-C₆-alkylene group of the —O—C₂-C₆-alkylene-(C₁-C₆-alkyl)amine group, for example an ethylene, propylene, butylene, pentylene or hexylene group, may be combined independently of one another with each C₁-C₆-alkyl group on the amino group, for example a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —O—C₂-C₆-alkylene-[di(C₁-C₆-alkyl)]amine groups of the radicals R4 to R6, the C₂-C₆-alkylene group of the —O—C₂-C₆-alkylene-[di(C₁-C₆-alkyl)]amine group, for example an ethylene, propylene, butylene, pentylene or hexylene group, may be combined independently of one another with two freely selectable C₁-C₆-alkyl groups on the amino group, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl-, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

Compounds preferred according to the present invention are those of the formula II and III 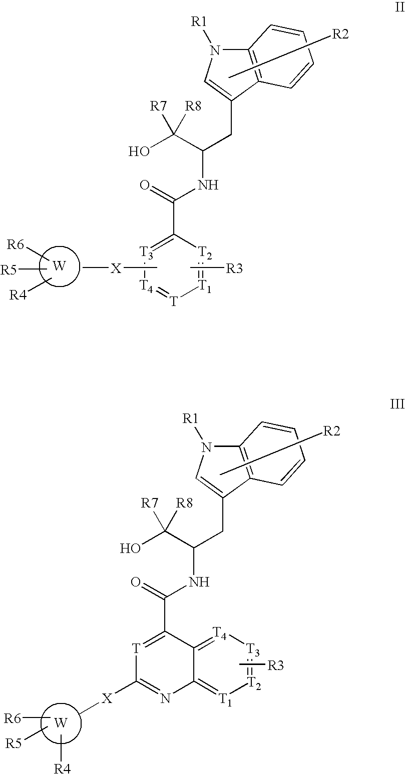
in which the radicals R1 to R8 and W have the same meaning as in formula I and
X is a bond, C₁-C₄-alkylene, C₂-C₄-alkenylene, C₂-C₄-alkynylene;
T is a nitrogen atom or a CH group;
T1, T2, T3, T4 are each independently of one another a nitrogen atom or an R3-C group.

Compounds likewise preferred according to the present invention are those of formula IIa and IIIa 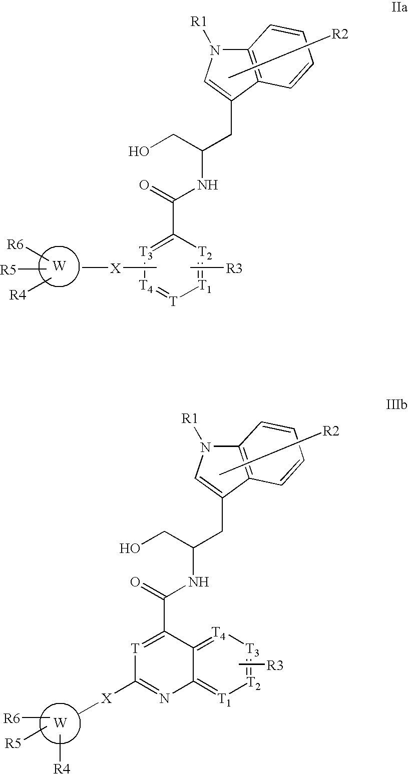
in which the radicals R1 to R6 and W have the same meaning as in formula Ia and
X is a bond, C₁-C₄-alkylene, C₂-C₄-alkenylene, C₂-C₄-alkynylene;
T is a nitrogen atom or a CH group;
T1, T2, T3, T4 are each independently of one another a nitrogen atom or an R3-C group.

Compounds particularly preferred according to the present invention are those of the formula IV and V 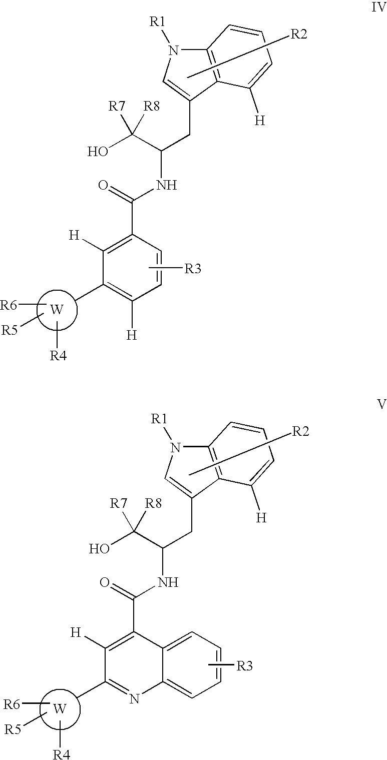
in which the radicals R1 to R8 and W have the same meaning as in formula I.

Compounds likewise particularly preferred according to the present invention are those of the formula IVa and Va 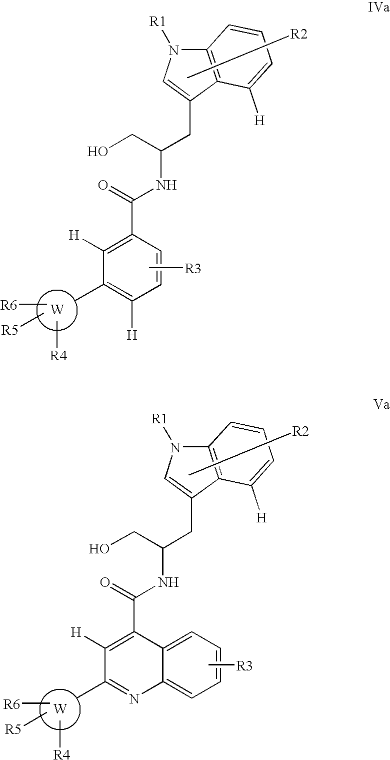
in which the radicals R1 to R6 and W have the same meaning as in formula Ia.

The following compounds are very particularly preferred:
1 N-[(R,S)-2-(5-Bromo-1H-indol-3-yl)-1-(hydroxymethyl)ethyl]-2-(3,4,5-trimethoxyphenyl)quinoline-4-carboxamide;
2 N-[(R,S)-1-(Hydroxymethyl)-2-(5-methyl-1H-indol-3-yl)ethyl]-2-(3,4,5-trimethoxyphenyl)quinoline-4-carboxamide;
3 N-[(R,S)-1-(Hydroxymethyl)-2-(4-methyl-1H-indol-3-yl)ethyl]-2-(3,4,5-trimethoxyphenyl)quinoline-4-carboxamide;
4 N-[(R,S)-1-(Hydroxymethyl)-2-(6-methyl-1H-indol-3-yl)ethyl]-2-(3,4,5-trimethoxyphenyl)quinoline-4-carboxamide;
5 N-[(R)-1-(Hydroxymethyl)-2-(1-methyl-1H-indol-3-yl)ethyl]-2-(3,4,5-trimethoxyphenyl)quinoline-4-carboxamide;
6 N-[(R,S)-2-(5-Fluoro-1H-indol-3-yl)-1-(hydroxymethyl)ethyl]-2-(3,4,5-trimethoxyphenyl)quinoline-4-carboxamide;
7 N-[(R,S)-1-(Hydroxymethyl)-2-(5-methoxy-1H-indol-3-yl)ethyl]-2-(3,4,5-trimethoxyphenyl)quinoline-4-carboxamide;
8 N-[(R,S)-2-(6-Fluoro-1H-indol-3-yl)-1-(hydroxymethyl)ethyl]-2-(3,4,5-trimethoxyphenyl)quinoline-4-carboxamide;
9 N-[(R,S)-1-(Hydroxymethyl)-2-[5-(phenylmethoxy)-1H-indol-3-yl]ethyl]-2-(3,4,5-trimethoxyphenyl)quinoline-4-carboxamide;
10 N-[(R,S)-1-(Hydroxymethyl)-2-(7-methyl-1H-indol-3-yl)ethyl]-2-(3,4,5-trimethoxyphenyl)quinoline-4-carboxamide;
11 N-[(R)-1-(Hydroxymethyl)-2-(1H-indol-3-yl)ethyl]-2-(3,4,5-trimethoxyphenyl)quinoline-4-carboxamide;
12 N-[(S)-1-(Hydroxymethyl)-2-(1H-indol-3-yl)ethyl]-2-(3,4,5-trimethoxyphenyl)quinoline-4-carboxamide;
13 2-(4-Chloro-3-methylphenyl)-N-[(R)-1-(hydroxymethyl)-2-(1H-indol-3-yl)ethyl]quinoline-4-carboxamide;
14 N-[(R)-1-(Hydroxymethyl)-2-(1H-indol-3-yl)ethyl]-6-methoxy-2-(3,4,5-trimethoxyphenyl)quinoline-4-carboxamide;
15 6-Bromo-N-[(R)-1-(hydroxymethyl)-2-(1H-indol-3-yl)ethyl]-2-(3,4,5-trimethoxyphenyl)quinoline-4-carboxamide;
16 N-[(R)-1-(Hydroxymethyl)-2-(1H-indol-3-yl)ethyl]-6-methoxy-2-(2,3,4-trimethoxyphenyl)quinoline-4-carboxamide;
17 6-Fluoro-N-[(R)-1-(hydroxymethyl)-2-(1H-indol-3-yl)ethyl]-2-(3,4,5-trimethoxyphenyl)quinoline-4-carboxamide;
18 N-[(R)-1-(Hydroxymethyl)-2-(1H-indol-3-yl)ethyl]-6-iodo-2-(3,4,5-trimethoxyphenyl)quinoline-4-carboxamide;
19 N-[(R)-1-(Hydroxymethyl)-2-(1H-indol-3-yl)ethyl]-6-nitro-2-(3,4,5-trimethoxyphenyl)quinoline-4-carboxamide;
20 6-Amino-N-[(R)-1-(hydroxymethyl)-2-(1H-indol-3-yl)ethyl]-2-(3,4,5-trimethoxyphenyl)quinoline-4-carboxamide;
21 N-[(R,S)-1-(Hydroxymethyl)-2-(5-fluoro-1H-indol-3-yl)ethyl]-6-methoxy-2-(3,4,5-trimethoxyphenyl)quinoline-4-carboxamide;
22 N-[(R)-1-(Hydroxymethyl)-2-(1-methyl-1H-indol-3-yl)ethyl]-6-methoxy-2-(3,4,5-trimethoxyphenyl)quinoline-4-carboxamide;
23 N-[(R,S)-2-(6-Fluoro-1H-indol-3-yl)-1-(hydroxymethyl)ethyl]-6-methoxy-2-(3,4,5-trimethoxyphenyl)quinoline-4-carboxamide;
24 2-(3,4-Dimethoxyphenyl)-N-[(S)-1-(hydroxymethyl)-2-(1H-indol-3-yl)ethyl]quinoline-4-carboxamide;
25 2-(3,4-Dimethoxyphenyl)-N-[(R)-1-(hydroxymethyl)-2-(1H-indol-3-yl)ethyl]quinoline-4-carboxamide;
26 2-(3,4-Dimethylphenyl)-N-[(R)-1-(hydroxymethyl)-2-(1H-indol-3-yl)ethyl]quinoline-4-carboxamide;
27 2-(2,3-Dihydro-1,4-benzodioxin-6-yl)-N-[(R)-1-(hydroxymethyl)-2-(1H-indol-3-yl)ethyl]-quinoline-4-carboxamide;
28 N-[(R)-1-(Hydroxymethyl)-2-(1H-indol-3-yl)ethyl]-2-[4-(trifluoromethoxy)phenyl]quinoline-4-carboxamide;
29 N-[(R)-1-(Hydroxymethyl)-2-(1H-indol-3-yl)ethyl]-2-[4-(methylsulphanyl)phenyl]quinoline-4-carboxamide;
30 2-(3,5-Dimethoxyphenyl)-N-[(R)-1-(hydroxymethyl)-2-(1H-indol-3-yl)ethyl]quinoline-4-carboxamide;
31 2-[3-(Acetylamino)phenyl]-N-[(R)-1-(hydroxymethyl)-2-(1H-indol-3-yl)ethyl]quinoline-4-carboxamide;
32 2-(4-Chlorophenyl)-N-[(R)-1-(hydroxymethyl)-2-(1H-indol-3-yl)ethyl]quinoline-4-carboxamide;
33 N-[(R)-1-(Hydroxymethyl)-2-(1H-indol-3-yl)ethyl]-2-(4-methoxyphenyl)quinoline-4-carboxamide;
34 N-[(R)-1-(Hydroxymethyl)-2-(1H-indol-3-yl)ethyl]-2-(3-methoxyphenyl)quinoline-4-carboxamide;
35 N-[(R)-2-(1-Ethyl-1H-indol-3-yl)-1-(hydroxymethyl)ethyl]-6-methoxy-2-(3,4,5-trimethoxyphenyl)quinoline-4-carboxamide;
36 2-(2,3-Dihydrobenzofuran-5-yl)-N-[(R)-1-(hydroxymethyl)-2-(1H-indol-3-yl)ethyl]-quinoline-4-carboxamide;
37 N-[(R)-1-(Hydroxymethyl)-2-(1H-indol-3-yl)ethyl]-6-methoxy-2-(7-methoxybenzofuran-2-yl)quinoline-4-carboxamide;
38 2-[(Z)-2-(3,4-Dimethoxyphenyl)ethenyl]-N-[(R)-1-(hydroxymethyl)-2-(1H-indol-3-yl)ethyl]-6-methoxyquinoline-4-carboxamide;
39 N-[(R)-1-(Hydroxymethyl)-2-(1H-indol-3-yl)ethyl]-4′-methoxy[1,1′-biphenyl]-2-carboxamide;
40 N-[(S)-1-(Hydroxymethyl)-2-(1H-indol-3-yl)ethyl]-3′,4′-dimethoxy[1,1′-biphenyl]-3-carboxamide;
41 N-[(R)-1-(Hydroxymethyl)-2-(1H-indol-3-yl)ethyl]-3′,4′-dimethoxy[1,1′-biphenyl]-3-carboxamide;
42 N-[(R)-1-(Hydroxymethyl)-2-(1H-indol-3-yl)ethyl]-2′,3′,4′-trimethoxy[1,1′-biphenyl]-3-carboxamide;
43 N-[(R)-1-(Hydroxymethyl)-2-(1H-indol-3-yl)ethyl]-3′,4′,5′-trimethoxy[1,1′-biphenyl]-3-carboxamide;
44 N-[(R)-1-(Hydroxymethyl)-2-(1H-indol-3-yl)ethyl]-3′,4′,5′-trimethoxy[1,1′-biphenyl]-4-carboxamide;
45 N-[(R)-1-(Hydroxymethyl)-2-(1H-indol-3-yl)ethyl]-3′,4′,5′-trimethoxy-2-methyl[1,1′-biphenyl]-4-carboxamide;
46 N-[(R)-1-(Hydroxymethyl)-2-(1H-indol-3-yl)ethyl]-2′,3′,4′-trimethoxy[1,1′-biphenyl]-2-carboxamide;
47 N-[(R)-1-(Hydroxymethyl)-2-(1H-indol-3-yl)ethyl]-3′,4′,5′-trimethoxy[1,1′-biphenyl]-2-carboxamide;
48 N-[(R)-1-(Hydroxymethyl)-2-(1H-indol-3-yl)ethyl]-2′,3′,4′-trimethoxy-6-methyl[1,1′-biphenyl]-3-carboxamide;
49 N-[(R)-1-(Hydroxymethyl)-2-(1H-indol-3-yl)ethyl]-3′,4′,5′-trimethoxy-6-methyl[1,1′-biphenyl]-3-carboxamide;
50 N-[(R)-1-(Hydroxymethyl)-2-(1H-indol-3-yl)ethyl]-2′,3′,4′-trimethoxy[1,1′-biphenyl]-4-carboxamide;
51 N-[(R)-1-(Hydroxymethyl)-2-(1H-indol-3-yl)ethyl]-2′,3′,4′-trimethoxy-2-methyl[1,1′-biphenyl]-4-carboxamide;
52 N-[(R)-1-(Hydroxymethyl)-2-(1H-indol-3-yl)ethyl]-2′,3′,4,4′-tetramethoxy[1,1′-biphenyl]-2-carboxamide;
53 N-[(R)-1-(Hydroxymethyl)-2-(1H-indol-3-yl)ethyl]-3′,4,4′,5′-tetramethoxy[1,1′-biphenyl]-2-carboxamide;
54 4′-(Hydroxymethyl)-N-[(R)-1-(hydroxymethyl)-2-(1H-indol-3-yl)ethyl]-6-methyl[1,1′-biphenyl]-3-carboxamide;
55 4′-(Hydroxymethyl)-N-[(R)-1-(hydroxymethyl)-2-(1H-indol-3-yl)ethyl]-2-methyl[1,1′-biphenyl]-4-carboxamide;
56 4′-(Hydroxymethyl)-N-[(R)-1-(hydroxymethyl)-2-(1H-indol-3-yl)ethyl][1,1′-biphenyl]-2-carboxamide;
57 4′-(Hydroxymethyl)-N-[(R)-1-(hydroxymethyl)-2-(1H-indol-3-yl)ethyl][1,1′-biphenyl]-3-carboxamide;
58 N-[(R)-1-(Hydroxymethyl)-2-(1H-indol-3-yl)ethyl]-4-methoxy-3′-(1-methylethyl)[1,1′-biphenyl]-2-carboxamide;
59 N-[(R)-1-(Hydroxymethyl)-2-(1H-indol-3-yl)ethyl]-3′-(1-methylethyl)[1,1′-biphenyl]-3-carboxamide;
60 N-[(R)-1-(Hydroxymethyl)-2-(1H-indol-3-yl)ethyl]-6-methyl-3′-(1-methylethyl)[1,1′-biphenyl]-3-carboxamide;
61 N-[(R)-1-(Hydroxymethyl)-2-(1H-indol-3-yl)ethyl]-3′-(1-methylethyl)[1,1′-biphenyl]-4-carboxamide;
62 N-[(R)-1-(Hydroxymethyl)-2-(1H-indol-3-yl)ethyl]-2-methyl-3′-(1-methylethyl)[1,1′-biphenyl]-4-carboxamide;
63 4′-(Hydroxymethyl)-N-[(R)-1-(hydroxymethyl)-2-(1H-indol-3-yl)ethyl]-4-methoxy[1,1′-biphenyl]-2-carboxamide;
64 3′,4′,5′-Trifluoro-N-[(R)-1-(hydroxymethyl)-2-(1H-indol-3-yl)ethyl][1,1′-biphenyl]-2-carboxamide;
65 3′,4′,5′-Trifluoro-N-[(R)-1-(hydroxymethyl)-2-(1H-indol-3-yl)ethyl][1,1′-biphenyl]-3-carboxamide;
66 3′,4′,5′-Trifluoro-N-[(R)-1-(hydroxymethyl)-2-(1H-indol-3-yl)ethyl]-6-methyl[1,1′-biphenyl]-3-carboxamide;
67 3′,4′,5′-Trifluoro-N-[(R)-1-(hydroxymethyl)-2-(1H-indol-3-yl)ethyl][1,1′-biphenyl]-4-carboxamide;
68 3′,4′,5′-Trifluoro-N-[(R)-1-(hydroxymethyl)-2-(1H-indol-3-yl)ethyl]-2-methyl[1,1′-biphenyl]-4-carboxamide;
69 N-[(R)-1-(Hydroxymethyl)-2-(1H-indol-3-yl)ethyl]-2′,5′-dimethoxy[1,1′-biphenyl]-2-carboxamide;
70 N-[(R)-1-(Hydroxymethyl)-2-(1H-indol-3-yl)ethyl]-2′,4,5′-trimethoxy[1,1′-biphenyl]-2-carboxamide;
71 N-[(R)-1-(Hydroxymethyl)-2-(1H-indol-3-yl)ethyl]-2′,5′-dimethoxy-6-methyl[1,1′-biphenyl]-3-carboxamide;
72 N-[(R)-1-(Hydroxymethyl)-2-(1H-indol-3-yl)ethyl]-2′,5′-dimethoxy[1,1′-biphenyl]-4-carboxamide;
73 N-[(R)-1-(Hydroxymethyl)-2-(1H-indol-3-yl)ethyl]-2′,5′-dimethoxy-2-methyl[1,1′-biphenyl]-4-carboxamide;
74 N-[(R)-1-(Hydroxymethyl)-2-(1H-indol-3-yl)ethyl]-3′,4′-dimethoxy[1,1′-biphenyl]-2-carboxamide;
75 N-[(R)-1-(Hydroxymethyl)-2-(1H-indol-3-yl)ethyl]-3′,4,4′-trimethoxy[1,1′-biphenyl]-2-carboxamide;
76 N-[(R)-1-(Hydroxymethyl)-2-(1H-indol-3-yl)ethyl]-3′,4′-dimethoxy-6-methyl[1,1′-biphenyl]-3-carboxamide;
77 N-[(R)-1-(Hydroxymethyl)-2-(1H-indol-3-yl)ethyl]-3′,4′-dimethoxy[1,1′-biphenyl]-4-carboxamide;
78 N-[(R)-1-(Hydroxymethyl)-2-(1H-indol-3-yl)ethyl]-3′,4′-dimethoxy-2-methyl[1,1′-biphenyl]-4-carboxamide;
79 3′-Fluoro-N-[(R)-1-(hydroxymethyl)-2-(1H-indol-3-yl)ethyl]-4′-methoxy[1,1′-biphenyl]-2-carboxamide;
80 3′-Fluoro-N-[(R)-1-(hydroxymethyl)-2-(1H-indol-3-yl)ethyl]-4,4′-dimethoxy[1,1′-biphenyl]-2-carboxamide;
81 3′-Fluoro-N-[(R)-1-(hydroxymethyl)-2-(1H-indol-3-yl)ethyl]-4′-methoxy[1,1′-biphenyl]-3-carboxamide;
82 3′-Fluoro-N-[(R)-1-(hydroxymethyl)-2-(1H-indol-3-yl)ethyl]-4′-methoxy-6-methyl[1,1′-biphenyl]-3-carboxamide;
83 3′-Fluoro-N-[(R)-1-(hydroxymethyl)-2-(1H-indol-3-yl)ethyl]-4′-methoxy[1,1′-biphenyl]-4-carboxamide;
84 3′-Fluoro-N-[(R)-1-(hydroxymethyl)-2-(1H-indol-3-yl)ethyl]-4′-methoxy-2-methyl[1,1′-biphenyl]-4-carboxamide;
85 N-[(R)-1-(Hydroxymethyl)-2-(1H-indol-3-yl)ethyl]-3′,4-dimethoxy[1,1′-biphenyl]-2-carboxamide;
86 N-[(R)-1-(Hydroxymethyl)-2-(1H-indol-3-yl)ethyl]-3′-(1-methylethyl)[1,1′-biphenyl]-2-carboxamide;
87 N-[(R)-1-(Hydroxymethyl)-2-(1H-indol-3-yl)ethyl]-2′,5′-dimethoxy[1,1′-biphenyl]-3-carboxamide;
88 3′,4′,5′-Trifluoro-N-[(R)-1-(hydroxymethyl)-2-(1H-indol-3-yl)ethyl]-4-methoxy[1,1′-biphenyl]-2-carboxamide;
89 3-(Benzofuran-2-yl)-N-[(R)-1-(hydroxymethyl)-2-(1H-indol-3-yl)ethyl]benzamide;
90 N-[(R)-1-(Hydroxymethyl)-2-(1H-indol-3-yl)ethyl]-3-(5-methoxybenzofuran-2-yl)benzamide;
91 N-[(R)-1-(Hydroxymethyl)-2-(1H-indol-3-yl)ethyl]-2-[(3,4,5-trimethoxyphenyl)methoxy]-phenylpropanamide
92 N-[(R)-1-(Hydroxymethyl)-2-(1H-indol-3-yl)ethyl]-4-[[(3,4,5-trimethoxyphenyl)methoxy]-methyl]benzamide;
93 N-[(R)-1-(Hydroxymethyl)-2-(1H-indol-3-yl)ethyl]-3-[(3,4,5-trimethoxyphenyl)methoxy]-thiophene-2-carboxamide;
94 N-[(R)-1-(Hydroxymethyl)-2-(1H-indol-3-yl)ethyl]-4-[(3,4,5-trimethoxyphenyl)methoxy]-phenylacetamide;
95 N-[(R)-1-(Hydroxymethyl)-2-(1H-indol-3-yl)ethyl]-3-[(3,4,5-trimethoxyphenyl)methoxy]-phenylpropanamide;
96 2-[2-(3,4-Dimethoxyphenyl)ethyl]-N-[(R)-1-(hydroxymethyl)-2-(1H-indol-3-yl)ethyl]-6-methoxyquinoline-4-carboxamide;
487 N-[(R)-1-(Hydroxymethyl)-2-(1H-indol-3-yl)ethyl]-2-(3,4,5-trimethoxyphenyl)-1,6-naphthyridine-4-carboxamide;
488 6-Bromo-N-[(R)-1-(hydroxymethyl)-2-(1H-indol-3-yl)ethyl]-2-(3,4,5-trimethoxyphenyl)-1,8-naphthyridine-4-carboxamide;
97 2-(6-Methoxynaphthalen-2-yl)quinoline-4-carboxylic acid [(R)-1-hydroxymethyl-2-(1H-indol-3-yl)ethyl]amide;
98 6-Methoxy-2-(3-methoxyphenyl)quinoline-4-carboxylic acid [(R)-1-hydroxymethyl-2-(1H-indol-3-yl)ethyl]amide;
99 2-(4-Fluoro-3-methoxyphenyl)-6-methoxyquinoline-4-carboxylic acid [(R)-2-hydroxy-1-(1H-indol-3-ylmethyl)ethyl]amide;
100 2-(3-Iodo-4-methoxyphenyl)-6-methoxyquinoline-4-carboxylic acid [(R)-2-hydroxy-1-(1H-indol-3-ylmethyl)ethyl]amide;
101 2-(3-Hydroxyphenyl)-6-methoxyquinoline-4-carboxylic acid [(R)-1-hydroxymethyl-2-(1H-indol-3-yl)ethyl]amide;
102 2-(4-Hydroxy-3,5-dimethoxyphenyl)-6-methoxyquinoline-4-carboxylic acid [(R)-1-hydroxymethyl-2-(1H-indol-3-yl)ethyl]amide;
103 2-(3,5-Difluoro-4-methoxyphenyl)-6-methoxyquinoline-4-carboxylic acid [(R)-2-hydroxy-1-(1H-indol-3-ylmethyl)ethyl]amide;
104 2-(3-Ethylphenyl)-6-methoxyquinoline-4-carboxylic acid [(R)-1-hydroxymethyl-2-(1H-indol-3-yl)ethyl]amide;
105 2-(3-Fluoro-4-methoxyphenyl)-6-methoxyquinoline-4-carboxylic acid [(R)-2-hydroxy-1-(1H-indol-3-ylmethyl)ethyl]amide;
106 2-(3-Fluoro-4-methoxyphenyl)-6-methylquinoline-4-carboxylic acid [(R)-2-hydroxy-1-(1H-indol-3-ylmethyl)ethyl]amide;
107 6-Methyl-2-(3,4,5-trimethoxyphenyl)quinoline-4-carboxylic acid [(R)-1-hydroxymethyl-2-(1H-indol-3-yl)ethyl]amide;
108 6-Bromo-2-(2,4-dimethylthiazol-5-yl)quinoline-4-carboxylic acid [(R)-2-hydroxy-1-(1H-indol-3-ylmethyl)ethyl]amide;
109 2-(7-Methoxybenzofuran-2-yl)-6-trifluoromethoxyquinoline-4-carboxylic acid [(R)-2-hydroxy-1-(1H-indol-3-ylmethyl)ethyl]amide;
110 2-(3-Fluoro-4-methoxyphenyl)-6-iodoquinoline-4-carboxylic acid [(R)-2-hydroxy-1-(1H-indol-3-ylmethyl)ethyl]amide;
111 2-(3-Fluoro-4-methoxyphenyl)-6-trifluoromethoxyquinoline-4-carboxylic acid [(R)-2-hydroxy-1-(1H-indol-3-ylmethyl)ethyl]amide;
112 2-(3-Fluoro-4-methoxyphenyl)-6,8-dimethylquinoline-4-carboxylic acid [(R)-2-hydroxy-1-(1H-indol-3-ylmethyl)ethyl]amide;
113 2-(3,4-Dimethoxyphenyl)-6-methoxy-3-methylquinoline-4-carboxylic acid [(R)-1-hydroxymethyl-2-(1H-indol-3-yl)ethyl]amide;
114 6-Amino-2-(3-fluoro-4-methoxyphenyl)quinoline-4-carboxylic acid [(R)-2-hydroxy-1-(1H-indol-3-ylmethyl)ethyl]amide;
115 2-(4,6-Dimethoxybenzofuran-2-yl)-N-[(R)-1-(hydroxymethyl)-2-(1H-indol-3-yl)ethyl]-6-methoxyquinoline-4-carboxamide;
116 N-[(R)-1-(Hydroxymethyl)-2-(1H-indol-3-yl)ethyl]-6-methoxy-2-(5-methoxybenzofuran-2-yl)quinoline-4-carboxamide;
117 2-(7-Ethoxybenzofuran-2-yl)-N-[(R)-1-(hydroxymethyl)-2-(1H-indol-3-yl)ethyl]-6-methoxyquinoline-4-carboxamide;
118 N-[(R)-1-(Hydroxymethyl)-2-(1H-indol-3-yl)ethyl]-6-methoxy-2-(6-methoxybenzofuran-2-yl)quinoline-4-carboxamide;
119 2-(7-Fluorbenzofuran-2-yl)-N-[(R)-1-(hydroxymethyl)-2-(1H-indol-3-yl)ethyl]-6-methoxyquinoline-4-carboxamide;
120 2-(4-Fluorbenzofuran-2-yl)-N-[(R)-1-(hydroxymethyl)-2-(1H-indol-3-yl)ethyl]-6-methoxyquinoline-4-carboxamide;
121 N-[(R)-1-(Hydroxymethyl)-2-(1H-indol-3-yl)ethyl]-6-methoxy-2-(5-methylbenzofuran-2-yl)quinoline-4-carboxamide;
122 N-[(R)-1-(Hydroxymethyl)-2-(1H-indol-3-yl)ethyl]-6-methoxy-2-(7-methylbenzofuran-2-yl)quinoline-4-carboxamide;
123 N-[(R)-1-(Hydroxymethyl)-2-(1H-indol-3-yl)ethyl]-6-methoxy-2-(4-methoxybenzofuran-2-yl)quinoline-4-carboxamide;
124 N-[(R)-1-(Hydroxymethyl)-2-(1H-indol-3-yl)ethyl]-6-methoxy-2-[5-(trifluoromethoxy)benzofuran-2-yl]quinoline-4-carboxamide;
125 4-Ethoxybiphenyl-3-carboxylic acid [(R)-1-hydroxymethyl-2-(1H-indol-3-yl)ethyl]amide;
126 4-Ethoxy-3′-fluoro-4′-propoxybiphenyl-3-carboxylic acid [(R)-2-hydroxy-1-(1H-indol-3-ylmethyl)ethyl]amide;
127 2-Ethoxy-N-[(R)-1-hydroxymethyl-2-(1H-indol-3-yl)ethyl]-5-(6-methoxypyridin-3-yl)benzamide;
128 4-Ethoxy-2′-fluoro-3′-methoxybiphenyl-3-carboxylic acid [(R)-2-hydroxy-1-(1H-indol-3-ylmethyl)ethyl]amide;
129 4′-Acetylamino-4-ethoxybiphenyl-3-carboxylic acid [(R)-1-hydroxymethyl-2-(1H-indol-3-yl)ethyl]amide;
130 2-Ethoxy-N-[(R)-1-hydroxymethyl-2-(1H-indol-3-yl)ethyl]-5-(2-methoxypyrimidin-5-yl)benzamide;
131 4-Ethoxy-5′-fluoro-3′-methoxybiphenyl-3-carboxylic acid [(R)-2-hydroxy-1-(1H-indol-3-ylmethyl)ethyl]amide;
132 4-Ethoxy-3′,4′-difluoro-5′-methoxybiphenyl-3-carboxylic acid [(R)-2-hydroxy-1-(1H-indol-3-ylmethyl)ethyl]amide;
133 4-Ethoxy-4′-fluoro-3′-methoxybiphenyl-3-carboxylic acid [(R)-2-hydroxy-1-(1H-indol-3-ylmethyl)ethyl]amide;
134 3′,5′-Dimethoxy-4-propoxybiphenyl-3-carboxylic acid [(R)-1-hydroxymethyl-2-(1H-indol-3-yl)ethyl]amide;
135 4-Ethoxy-3′-hydroxymethylbiphenyl-3-carboxylic acid [(R)-1-hydroxymethyl-2-(1H-indol-3-yl)ethyl]amide;
136 4-Ethoxy-3′-methylsulphanylbiphenyl-3-carboxylic acid [(R)-2-hydroxy-1-(1H-indol-3-ylmethyl)ethyl]amide;
137 3′-Cyano-4-ethoxybiphenyl-3-carboxylic acid [(R)-1-hydroxymethyl-2-(1H-indol-3-yl)ethyl]amide;
138 2-Ethoxy-5-(6-fluoro-5-methylpyridin-3-yl)-N-[(R)-2-hydroxy-1-(1H-indol-3-ylmethyl)ethyl]benzamide;
139 4-Ethoxy-4′-trifluoromethoxybiphenyl-3-carboxylic acid [(R)-2-hydroxy-1-(1H-indol-3-ylmethyl)ethyl]amide;
140 5-Benzo[b]thiophene-3-yl-2-ethoxy-N-[(R)-2-hydroxy-1-(1H-indol-3-ylmethyl)ethyl]benzamide;
141 4-Ethoxy-2′-trifluoromethylbiphenyl-3-carboxylic acid [(R)-2-hydroxy-1-(1H-indol-3-ylmethyl)ethyl]amide;
142 4-Ethoxy-2′-trifluoromethoxybiphenyl-3-carboxylic acid [(R)-2-hydroxy-1-(1H-indol-3-ylmethyl)ethyl]amide;
143 4-Ethoxy-3′-trifluoromethoxybiphenyl-3-carboxylic acid [(R)-2-hydroxy-1-(1H-indol-3-ylmethyl)ethyl]amide;
144 4-Ethoxy-3′-fluorobiphenyl-3-carboxylic acid [(R)-2-hydroxy-1-(1H-indol-3-ylmethyl)ethyl]amide;
145 4′-Chloro-4-ethoxybiphenyl-3-carboxylic acid [(R)-2-hydroxy-1-(1H-indol-3-ylmethyl)ethyl]amide;
146 4-Ethoxy-4′-methylsulphanylbiphenyl-3-carboxylic acid [(R)-2-hydroxy-1-(1H-indol-3-ylmethyl)ethyl]amide;
147 4-Ethoxy-3′-trifluoromethylbiphenyl-3-carboxylic acid [(R)-2-hydroxy-1-(1H-indol-3-ylmethyl)ethyl]amide;
148 3′-Chloro-4-ethoxybiphenyl-3-carboxylic acid [(R)-2-hydroxy-1-(1H-indol-3-ylmethyl)ethyl]amide;
149 4-Ethoxy-3′-methylbiphenyl-3-carboxylic acid [(R)-1-hydroxymethyl-2-(1H-indol-3-yl)ethyl]amide;
150 5-Benzofuran-2-yl-2-ethoxy-N-[(R)-1-hydroxymethyl-2-(1H-indol-3-yl)ethyl]benzamide;
151 4-Ethoxy-2′-methylsulphanylbiphenyl-3-carboxylic acid [(R)-2-hydroxy-1-(1H-indol-3-ylmethyl)ethyl]amide;
152 2-Ethoxy-N-[(R)-1-hydroxymethyl-2-(1H-indol-3-yl)ethyl]-5-(1H-indol-4-yl)benzamide;
153 2-Ethoxy-N-[(R)-2-hydroxy-1-(1H-indol-3-ylmethyl)ethyl]-5-(4-methylthiophen-2-yl)benzamide;
154 3′-Acetylamino-4-ethoxybiphenyl-3-carboxylic acid [(R)-1-hydroxymethyl-2-(1H-indol-3-yl)ethyl]amide;
155 4-Ethoxy-2′-methylbiphenyl-3-carboxylic acid [(R)-1-hydroxymethyl-2-(1H-indol-3-yl)ethyl]amide;
156 2-Ethoxy-N-[(R)-1-hydroxymethyl-2-(1H-indol-3-yl)ethyl]-5-(5-methylfuran-2-yl)benzamide;
157 3′-Chloro-4-ethoxy-4′-methylbiphenyl-3-carboxylic acid [(R)-2-hydroxy-1-(1H-indol-3-ylmethyl)ethyl]amide;
158 5-(2-Chloro-6-methylpyridin-3-yl)-2-ethoxy-N-[(R)-2-hydroxy-1-(1H-indol-3-ylmethyl)ethyl]benzamide;
159 4-Ethoxy-4′-fluorobiphenyl-3-carboxylic acid [(R)-2-hydroxy-1-(1H-indol-3-ylmethyl)ethyl]amide;
160 2-Ethoxy-N-[(R)-1-hydroxymethyl-2-(1H-indol-3-yl)ethyl]-5-naphthalen-1-yl-benzamide;
161 5-Benzo[b]thiophene-2-yl-2-ethoxy-N-[(R)-2-hydroxy-1-(1H-indol-3-ylmethyl)ethyl]benzamide;
162 4-Ethoxy-4′-methylbiphenyl-3-carboxylic acid [(R)-1-hydroxymethyl-2-(1H-indol-3-yl)ethyl]amide;
163 2-Ethoxy-N-[(R)-2-hydroxy-1-(1H-indol-3-ylmethyl)ethyl]-5-thiophen-3-yl-benzamide;
164 4-Ethoxy-4′-methoxybiphenyl-3-carboxylic acid [(R)-1-hydroxymethyl-2-(1H-indol-3-yl)ethyl]amide;
165 2′,4′-Dichloro-4-ethoxybiphenyl-3-carboxylic acid [(R)-2-hydroxy-1-(1H-indol-3-ylmethyl)ethyl]amide;
166 4′-Methoxy-4-propoxybiphenyl-3-carboxylic acid [(R)-1-hydroxymethyl-2-(1H-indol-3-yl)ethyl]amide;
167 4-Propoxybiphenyl-3-carboxylic acid [(R)-1-hydroxymethyl-2-(1H-indol-3-yl)ethyl]amide;
168 5-Benzofuran-2-yl-N-[(R)-1-hydroxymethyl-2-(1H-indol-3-yl)ethyl]-2-propoxybenzamide;
169 3′-Chloro-4-propoxybiphenyl-3-carboxylic acid [(R)-2-hydroxy-1-(1H-indol-3-ylmethyl)ethyl]amide;
170 5-Benzo[b]thiophene-2-yl-N-[(R)-2-hydroxy-1-(1H-indol-3-ylmethyl)ethyl]-2-propoxybenzamide;
171 3′-Fluoro-4′-methyl-4-propoxybiphenyl-3-carboxylic acid [(R)-2-hydroxy-1-(1H-indol-3-ylmethyl)ethyl]amide;
172 4-Propoxy-3′-trifluoromethylbiphenyl-3-carboxylic acid [(R)-2-hydroxy-1-(1H-indol-3-ylmethyl)ethyl]amide;
173 2′-Fluoro-5′-methoxy-4-propoxybiphenyl-3-carboxylic acid [(R)-2-hydroxy-1-(1H-indol-3-ylmethyl)ethyl]amide;
174 4-Propoxy-3′,5′-bis-trifluoromethylbiphenyl-3-carboxylic acid [(R)-2-hydroxy-1-(1H-indol-3-ylmethyl)ethyl]amide;
175 4′-Chloro-4-propoxybiphenyl-3-carboxylic acid [(R)-2-hydroxy-1-(1H-indol-3-ylmethyl)ethyl]amide;
176 5-Benzo[b]thiophene-3-yl-N-[(R)-2-hydroxy-1-(1H-indol-3-ylmethyl)ethyl]-2-propoxybenzamide;
177 4-Propoxy-4′-trifluoromethylbiphenyl-3-carboxylic acid [(R)-2-hydroxy-1-(1H-indol-3-ylmethyl)ethyl]amide;
178 3′-Hydroxy-4-propoxybiphenyl-3-carboxylic acid [(R)-1-hydroxymethyl-2-(1H-indol-3-yl)ethyl]amide;
179 N-[(R)-1-Hydroxymethyl-2-(1H-indol-3-yl)ethyl]-2-propoxy-5-quinolin-6-yl-benzamide;
180 5-(6-Fluoro-5-methylpyridin-3-yl)-N-[(R)-2-hydroxy-1-(1H-indol-3-ylmethyl)ethyl]-2-propoxybenzamide;
181 N-[(R)-1-Hydroxymethyl-2-(1H-indol-3-yl)ethyl]-5-(6-methoxypyridin-3-yl)-2-propoxybenzamide;
182 3′-Chloro-4′-methyl-4-propoxybiphenyl-3-carboxylic acid [(R)-2-hydroxy-1-(1H-indol-3-ylmethyl)ethyl]amide;
183 N-[(R)-1-Hydroxymethyl-2-(1H-indol-3-yl)ethyl]-2-propoxy-5-pyridin-4-yl-benzamide;
184 3′-Chloro-4′-fluoro-4-propoxybiphenyl-3-carboxylic acid [(R)-2-hydroxy-1-(1H-indol-3-ylmethyl)ethyl]amide;
185 3′-Acetylamino-4-propoxybiphenyl-3-carboxylic acid [(R)-1-hydroxymethyl-2-(1H-indol-3-yl)ethyl]amide;
186 3′,4′-Difluoro-4-propoxybiphenyl-3-carboxylic acid [(R)-2-hydroxy-1-(1H-indol-3-ylmethyl)ethyl]amide;
187 3′,5′-Difluoro-4-propoxybiphenyl-3-carboxylic acid [(R)-2-hydroxy-1-(1H-indol-3-ylmethyl)ethyl]amide;
188 3′-Cyano-4-propoxybiphenyl-3-carboxylic acid [(R)-1-hydroxymethyl-2-(1H-indol-3-yl)ethyl]amide;
189 5-(2,4-Dimethoxypyrimidin-5-yl)-N-[(R)-1-hydroxymethyl-2-(1H-indol-3-yl)ethyl]-2-propoxybenzamide;
190 2′,3′-Difluoro-4-propoxybiphenyl-3-carboxylic acid [(R)-2-hydroxy-1-(1H-indol-3-ylmethyl)ethyl]amide;
191 2′,5′-Difluoro-4-propoxybiphenyl-3-carboxylic acid [(R)-2-hydroxy-1-(1H-indol-3-ylmethyl)ethyl]amide;
192 5-[(E)-2-(4-Fluorophenyl)-vinyl]-N-[(R)-2-hydroxy-1-(1H-indol-3-ylmethyl)ethyl]-2-propoxybenzamide;
193 5-(5-Cyanothiophene-2-yl)-N-[(R)-2-hydroxy-1-(1H-indol-3-ylmethyl)ethyl]-2-propoxybenzamide;
194 2′-Fluoro-3′-methoxy-4-propoxybiphenyl-3-carboxylic acid [(R)-2-hydroxy-1-(1H-indol-3-ylmethyl)ethyl]amide;
195 N-[(R)-1-Hydroxymethyl-2-(1H-indol-3-yl)ethyl]-5-(2-methoxypyrimidin-5-yl)-2-propoxybenzamide;
196 4′-Chloro-2′,6′-difluoro-4-propoxybiphenyl-3-carboxylic acid [(R)-2-hydroxy-1-(1H-indol-3-ylmethyl)ethyl]amide;
197 3′,5′-Dimethyl-4-propoxybiphenyl-3-carboxylic acid [(R)-1-hydroxymethyl-2-(1H-indol-3-yl)ethyl]amide;
198 N-[(R)-1-Hydroxymethyl-2-(1H-indol-3-yl)ethyl]-2-propoxy-5-quinolin-3-yl-benzamide;
199 4′-Acetylamino-4-propoxybiphenyl-3-carboxylic acid [(R)-1-hydroxymethyl-2-(1H-indol-3-yl)ethyl]amide;
200 4-Propoxy-3′-(2,2,2-trifluoroethoxy)biphenyl-3-carboxylic acid [(R)-2-hydroxy-1-(1H-indol-3-ylmethyl)ethyl]amide;
201 3′-Ethoxy-5′-fluoro-4-propoxybiphenyl-3-carboxylic acid [(R)-2-hydroxy-1-(1H-indol-3-ylmethyl)ethyl]amide;
202 5′-Ethoxy-2′-fluoro-4-propoxybiphenyl-3-carboxylic acid [(R)-2-hydroxy-1-(1H-indol-3-ylmethyl)ethyl]amide;
203 3′-Ethoxy-4-propoxybiphenyl-3-carboxylic acid [(R)-1-hydroxymethyl-2-(1H-indol-3-yl)ethyl]amide;
204 4-Propoxybiphenyl-3-carboxylic acid [(R)-1-hydroxymethyl-2-(1-methyl-1H-indol-3-yl)ethyl]amide;
205 5-Benzofuran-2-yl-N-[(R)-1-hydroxymethyl-2-(1-methyl-1H-indol-3-yl)ethyl]-2-propoxybenzamide;
206 5-Benzo[b]thiophen-2-yl-N-[(R)-2-hydroxy-1-(1-methyl-1H-indol-3-ylmethyl)ethyl]-2-propoxybenzamide;
207 2′-Fluoro-4′-methyl-4-propoxybiphenyl-3-carboxylic acid [(R)-2-hydroxy-1-(1-methyl-1H-indol-3-ylmethyl)ethyl]amide;
208 4′-Fluoro-4-propoxybiphenyl-3-carboxylic acid [(R)-2-hydroxy-1-(1-methyl-1H-indol-3-ylmethyl)ethyl]amide;
209 2′-Fluoro-5′-methoxy-4-propoxybiphenyl-3-carboxylic acid [(R)-2-hydroxy-1-(1-methyl-1H-indol-3-ylmethyl)ethyl]amide;
210 3′-Fluoro-4-propoxybiphenyl-3-carboxylic acid [(R)-2-hydroxy-1-(1-methyl-1H-indol-3-ylmethyl)ethyl]amide;
211 N-[(R)-1-Hydroxymethyl-2-(1-methyl-1H-indol-3-yl)ethyl]-2-propoxy-5-pyridin-3-yl-benzamide;
212 5-Benzo[b]thiophen-3-yl-N-[(R)-2-hydroxy-1-(1-methyl-1H-indol-3-ylmethyl)ethyl]-2-propoxybenzamide;
213 3′-Cyano-4′-fluoro-4-propoxybiphenyl-3-carboxylic acid [(R)-2-hydroxy-1-(1-methyl-1H-indol-3-ylmethyl)ethyl]amide;
214 N-[(R)-1-Hydroxymethyl-2-(1-methyl-1H-indol-3-yl)ethyl]-5-(6-methoxypyridin-3-yl)-2-propoxybenzamide;
215 3′-Acetylamino-4-propoxybiphenyl-3-carboxylic acid [(R)-1-hydroxymethyl-2-(1-methyl-1H-indol-3-yl)ethyl]amide;
216 3′,4′-Difluoro-4-propoxybiphenyl-3-carboxylic acid [(R)-2-hydroxy-1-(1-methyl-1H-indol-3-ylmethyl)ethyl]amide;
217 3′,5′-Difluoro-4-propoxybiphenyl-3-carboxylic acid [(R)-2-hydroxy-1-(1-methyl-1H-indol-3-ylmethyl)ethyl]amide;
218 5-(2,4-Dimethoxypyrimidin-5-yl)-N-[(R)-1-hydroxymethyl-2-(1-methyl-1H-indol-3-yl)ethyl]-2-propoxybenzamide;
219 2′,5′-Difluoro-4-propoxybiphenyl-3-carboxylic acid [(R)-2-hydroxy-1-(1-methyl-1H-indol-3-ylmethyl)ethyl]amide;
220 5-[(E)-2-(4-Fluorophenyl)-vinyl]-N-[(R)-2-hydroxy-1-(1-methyl-1H-indol-3-ylmethyl)ethyl]-2-propoxybenzamide;
221 5-(5-Cyanothiophen-2-yl)-N-[(R)-2-hydroxy-1-(1-methyl-1H-indol-3-ylmethyl)ethyl]-2-propoxybenzamide;
222 N-[(R)-1-Hydroxymethyl-2-(1-methyl-1H-indol-3-yl)ethyl]-5-(2-methoxypyrimidin-5-yl)-2-propoxybenzamide;
223 N-[(R)-1-Hydroxymethyl-2-(1-methyl-1H-indol-3-yl)ethyl]-2-propoxy-5-quinoline-3-yl-benzamide;
224 5′-Fluoro-3′-methoxy-4-propoxybiphenyl-3-carboxylic acid [(R)-2-hydroxy-1-(1-methyl-1H-indol-3-ylmethyl)ethyl]amide;
225 4-Propoxy-3′-(2,2,2-trifluoroethoxy)biphenyl-3-carboxylic acid [(R)-2-hydroxy-1-(1-methyl-1H-indol-3-ylmethyl)ethyl]amide;
226 5′-Ethoxy-2′-fluoro-4-propoxybiphenyl-3-carboxylic acid [(R)-2-hydroxy-1-(1-methyl-1H-indol-3-ylmethyl)ethyl]amide;
227 4′-Methoxy-4-propoxybiphenyl-3-carboxylic acid [2-(5-fluoro-1H-indol-3-yl)-1-hydroxymethylethyl]amide;
228 5-Benzofuran-2-yl-N-[2-(5-fluoro-1H-indol-3-yl)-1-hydroxymethylethyl]-2-propoxybenzamide;
229 3′-Methyl-4-propoxybiphenyl-3-carboxylic acid [2-(5-fluoro-1H-indol-3-yl)-1-hydroxymethylethyl]amide;
230 5-Benzo[b]thiophen-2-yl-N-[2-(5-fluoro-1H-indol-3-yl)-1-hydroxymethylethyl]-2-propoxybenzamide;
231 2′-Fluoro-5′-methoxy-4-propoxybiphenyl-3-carboxylic acid [1-(5-fluoro-1H-indol-3-ylmethyl)-2-hydroxyethyl]amide;
232 4-Propoxy-3′,5′-bis-trifluoromethylbiphenyl-3-carboxylic acid [1-(5-fluoro-1H-indol-3-ylmethyl)-2-hydroxyethyl]amide;
233 N-[2-(5-Fluoro-1H-indol-3-yl)-1-hydroxymethylethyl]-2-propoxy-5-pyridin-3-yl-benzamide;
234 5-Benzo[b]thiophen-3-yl-N-[2-(5-fluoro-1H-indol-3-yl)-1-hydroxymethylethyl]-2-propoxybenzamide;
235 3′-Cyano-4′-fluoro-4-propoxybiphenyl-3-carboxylic acid [1-(5-fluoro-1H-indol-3-ylmethyl)-2-hydroxyethyl]amide;
236 N-[1-(5-Fluoro-1H-indol-3-ylmethyl)-2-hydroxyethyl]-5-(6-fluoro-5-methylpyridin-3-yl)-2-propoxybenzamide;
237 N-[2-(5-Fluoro-1H-indol-3-yl)-1-hydroxymethylethyl]-5-(6-methoxypyridin-3-yl)-2-propoxybenzamide;
238 3′-Chloro-4′-fluoro-4-propoxybiphenyl-3-carboxylic acid [1-(5-fluoro-1H-indol-3-ylmethyl)-2-hydroxyethyl]amide;
239 3′-Acetylamino-4-propoxybiphenyl-3-carboxylic acid [2-(5-fluoro-1H-indol-3-yl)-1-hydroxymethylethyl]amide;
240 3′,4′-Difluoro-4-propoxybiphenyl-3-carboxylic acid [1-(5-fluoro-1H-indol-3-ylmethyl)-2-hydroxyethyl]amide;
241 3′,5′-Difluoro-4-propoxybiphenyl-3-carboxylic acid [2-(5-fluoro-1H-indol-3-yl)-1-hydroxymethylethyl]amide;
242 2′,5′-Difluoro-4-propoxybiphenyl-3-carboxylic acid [1-(5-fluoro-1H-indol-3-ylmethyl)-2-hydroxyethyl]amide;
243 N-[2-(5-Fluoro-1H-indol-3-yl)-1-hydroxymethylethyl]-5-[(E)-2-(4-fluorophenyl)-vinyl]-2-propoxybenzamide;
244 5-(5-Cyanothiophen-2-yl)-N-[2-(5-fluoro-1H-indol-3-yl)-1-hydroxymethylethyl]-2-propoxybenzamide;
245 2′-Fluoro-3′-methoxy-4-propoxybiphenyl-3-carboxylic acid [1-(5-fluoro-1H-indol-3-ylmethyl)-2-hydroxyethyl]amide;
246 N-[2-(5-Fluoro-1H-indol-3-yl)-1-hydroxymethylethyl]-5-(2-methoxypyrimidin-5-yl)-2-propoxybenzamide;
247 N-[2-(5-Fluoro-1H-indol-3-yl)-1-hydroxymethylethyl]-2-propoxy-5-quinolin-3-yl-benzamide;
248 4-Propoxy-3′-(2,2,2-trifluoroethoxy)biphenyl-3-carboxylic acid [1-(5-fluoro-1H-indol-3-ylmethyl)-2-hydroxyethyl]amide;
249 5′-Ethoxy-2′-fluoro-4-propoxybiphenyl-3-carboxylic acid [1-(5-fluoro-1H-indol-3-ylmethyl)-2-hydroxyethyl]amide;
250 3′-Methoxy-4-propoxybiphenyl-3-carboxylic acid [(R)-1-hydroxymethyl-2-(1-methyl-1H-indol-3-yl)ethyl]amide;
251 3′-Chloro-4-propoxybiphenyl-3-carboxylic acid [(R)-2-hydroxy-1-(1-methyl-1H-indol-3-ylmethyl)ethyl]amide;
252 4-Propoxy-3′,5′-bis-trifluoromethylbiphenyl-3-carboxylic acid [(R)-2-hydroxy-1-(1-methyl-1H-indol-3-ylmethyl)ethyl]amide;
253 3′,4′,5′-Trifluoro-4-propoxybiphenyl-3-carboxylic acid [(R)-2-hydroxy-1-(1-methyl-1H-indol-3-ylmethyl)ethyl]amide;
254 4-Propoxy-4′-trifluoromethoxybiphenyl-3-carboxylic acid [(R)-2-hydroxy-1-(1-methyl-1H-indol-3-ylmethyl)ethyl]amide;
255 4-Propoxy-4′-trifluoromethylbiphenyl-3-carboxylic acid [(R)-2-hydroxy-1-(1-methyl-1H-indol-3-ylmethyl)ethyl]amide;
256 5-(6-Fluoro-5-methylpyridin-3-yl)-N-[(R)-2-hydroxy-1-(1-methyl-1H-indol-3-ylmethyl)ethyl]-2-propoxybenzamide;
257 5-(3,5-Dimethylisoxazol-4-yl)-N-[(R)-1-hydroxymethyl-2-(1-methyl-1H-indol-3-yl)ethyl]-2-propoxybenzamide;
258 3′-Chloro-4′-fluoro-4-propoxybiphenyl-3-carboxylic acid [(R)-2-hydroxy-1-(1-methyl-1H-indol-3-ylmethyl)ethyl]amide;
259 3′-Cyano-4-propoxybiphenyl-3-carboxylic acid [(R)-1-hydroxymethyl-2-(1-methyl-1H-indol-3-yl)ethyl]amide;
260 2′,3′-Difluoro-4-propoxybiphenyl-3-carboxylic acid [(R)-2-hydroxy-1-(1-methyl-1H-indol-3-ylmethyl)ethyl]amide;
261 3′,5′-Dimethyl-4-propoxybiphenyl-3-carboxylic acid [(R)-1-hydroxymethyl-2-(1-methyl-1H-indol-3-yl)ethyl]amide;
262 3′-Ethoxy-5′-fluoro-4-propoxybiphenyl-3-carboxylic acid [(R)-2-hydroxy-1-(1-methyl-1H-indol-3-ylmethyl)ethyl]amide;
263 5′-Fluoro-3′-hydroxy-4-propoxybiphenyl-3-carboxylic acid [(R)-2-hydroxy-1-(1-methyl-1H-indol-3-ylmethyl)ethyl]amide;
264 4,3′-Dipropoxybiphenyl-3-carboxylic acid [(R)-1-hydroxymethyl-2-(1-methyl-1H-indol-3-yl)ethyl]amide;
265 3′-Chloro-4-propoxybiphenyl-3-carboxylic acid [2-(5-fluoro-1H-indol-3-yl)-1-hydroxymethylethyl]amide;
266 3′-Fluoro-4′-methyl-4-propoxybiphenyl-3-carboxylic acid [1-(5-fluoro-1H-indol-3-ylmethyl)-2-hydroxyethyl]amide;
267 2′-Fluoro-4′-methyl-4-propoxybiphenyl-3-carboxylic acid [1-(5-fluoro-1H-indol-3-ylmethyl)-2-hydroxyethyl]amide;
268 4-Propoxy-3′-trifluoromethylbiphenyl-3-carboxylic acid [1-(5-fluoro-1H-indol-3-ylmethyl)-2-hydroxyethyl]amide;
269 3′-Isopropyl-4-propoxybiphenyl-3-carboxylic acid [2-(5-fluoro-1H-indol-3-yl)-1-hydroxymethylethyl]amide;
270 3′-Methylsulphanyl-4-propoxybiphenyl-3-carboxylic acid [2-(5-fluoro-1H-indol-3-yl)-1-hydroxymethylethyl]amide;
271 4-Propoxy-4′-trifluoromethoxybiphenyl-3-carboxylic acid [1-(5-fluoro-1H-indol-3-ylmethyl)-2-hydroxyethyl]amide;
272 N-[2-(5-Fluoro-1H-indol-3-yl)-1-hydroxymethylethyl]-2-propoxy-5-quinolin-6-yl-benzamide;
273 3′-Chloro-4′-methyl-4-propoxybiphenyl-3-carboxylic acid [2-(5-fluoro-1H-indol-3-yl)-1-hydroxymethylethyl]amide;
274 5-(3,5-Dimethylisoxazol-4-yl)-N-[2-(5-fluoro-1H-indol-3-yl)-1-hydroxymethylethyl]-2-propoxybenzamide;
275 2′,3′-Difluoro-4-propoxybiphenyl-3-carboxylic acid [1-(5-fluoro-1H-indol-3-ylmethyl)-2-hydroxyethyl]amide;
276 3′,5′-Dimethyl-4-propoxybiphenyl-3-carboxylic acid [2-(5-fluoro-1H-indol-3-yl)-1-hydroxymethylethyl]amide;
277 5′-Ethoxy-3′-fluoro-4-propoxybiphenyl-3-carboxylic acid [2-(5-fluoro-1H-indol-3-yl)-1-hydroxymethylethyl]amide;
278 3′-Fluoro-5′-hydroxy-4-propoxybiphenyl-3-carboxylic acid [2-(5-fluoro-1H-indol-3-yl)-1-hydroxymethylethyl]amide;
279 4,3′-Dipropoxybiphenyl-3-carboxylic acid [2-(5-fluoro-1H-indol-3-yl)-1-hydroxymethylethyl]amide;
280 3′-Ethoxy-4-propoxybiphenyl-3-carboxylic acid [2-(5-fluoro-1H-indol-3-yl)-1-hydroxymethylethyl]amide;
281 4′-Hydroxymethyl-4-propoxybiphenyl-3-carboxylic acid [(R)-1-hydroxymethyl-2-(1H-indol-3-yl)ethyl]amide;
282 3′-Hydroxymethyl-4-propoxybiphenyl-3-carboxylic acid [(R)-1-hydroxymethyl-2-(1H-indol-3-yl)ethyl]amide;
283 4-Propoxybiphenyl-3,4′-dicarboxylic acid 3-{[(R)-1-hydroxymethyl-2-(1H-indol-3-yl)ethyl]amide}4′-methylamide;
284 N-[(R)-2-Hydroxy-1-(1H-indol-3-ylmethyl)ethyl]-5-(5-hydroxymethylthiophen-2-yl)-2-propoxybenzamide;
285 5′-Fluoro-4-propoxybiphenyl-3,3′-dicarboxylic acid 3-{[(R)-2-hydroxy-1-(1H-indol-3-ylmethyl)ethyl]amide}3′-methylamide;
286 3′-Chloro-4-propoxybiphenyl-3,4′-dicarboxylic acid 3-{[(R)-2-hydroxy-1-(1H-indol-3-ylmethyl)ethyl]amide}4′-methylamide;
287 3′-Hydroxymethyl-4-propoxybiphenyl-3-carboxylic acid [(R)-1-hydroxymethyl-2-(1-methyl-1H-indol-3-yl)ethyl]amide;
288 3′-Hydroxymethyl-4-propoxybiphenyl-3-carboxylic acid [2-(5-fluoro-1H-indol-3-yl)-1-hydroxymethylethyl]amide;
289 3′-Chloro-4-propoxybiphenyl-3,4′-dicarboxylic acid 3-{[2-(5-fluoro-1H-indol-3-yl)-1-hydroxymethylethyl]amide}4′-methylamide;
290 N-[(R)-2-Hydroxy-1-(1-methyl-1H-indol-3-ylmethyl)ethyl]-5-(5-hydroxymethylthiophen-2-yl)-2-propoxybenzamide;
291 3′-Chloro-4-propoxybiphenyl-3,4′-dicarboxylic acid 3-{[(R)-2-hydroxy-1-(1-methyl-1H-indol-3-ylmethyl)ethyl]amide}4′-methylamide;
292 4′-Hydroxymethyl-4-propoxybiphenyl-3-carboxylic acid [2-(5-fluoro-1H-indol-3-yl)-1-hydroxymethylethyl]amide;
293 4-Propoxybiphenyl-3,4′-dicarboxylic acid 3-{[2-(5-fluoro-1H-indol-3-yl)-1-hydroxymethylethyl]amide}4′-methylamide;
294 5′-Fluoro-4-propoxybiphenyl-3,3′-dicarboxylic acid 3-{[2-(5-fluoro-1H-indol-3-yl)-1-hydroxymethylethyl]amide}3′-methylamide;
295 4-Ethoxy-3′-fluoro-N-[(R)-1-(hydroxymethyl)-2-(1H-indol-3-yl)ethyl]-4′-methoxy[1,1′-biphenyl]-3-carboxamide;
296 4-Ethoxy-N-[(R)-1-(hydroxymethyl)-2-(1H-indol-3-yl)ethyl]-3′-methoxy[1,1′-biphenyl]-3-carboxamide;
297 4-Ethoxy-N-[(R)-1-(hydroxymethyl)-2-(1H-indol-3-yl)ethyl]-N′-methyl[1,1′-biphenyl]-3,3′-dicarboxamide;
298 4-Ethoxy-N-[(R)-1-(hydroxymethyl)-2-(1H-indol-3-yl)ethyl]-3′,4′,5′-trimethoxy[1,1′-biphenyl]-3-carboxamide;
299 4-Ethoxy-N-[(R)-1-(hydroxymethyl)-2-(1H-indol-3-yl)ethyl]-3′,4′-dimethoxy[1,1′-biphenyl]-3-carboxamide;
300 4-Ethoxy-N-[(R)-1-(hydroxymethyl)-2-(1H-indol-3-yl)ethyl]-3′-(1-methylethyl)[1,1′-biphenyl]-3-carboxamide;
301 N-[(R)-1-(Hydroxymethyl)-2-(1H-indol-3-yl)ethyl]-3′,4′,5′-trimethoxy-4-propoxy[1,1′-biphenyl]-3-carboxamide;
302 N-[(R)-1-(Hydroxymethyl)-2-(1H-indol-3-yl)ethyl]-3′,4′-dimethoxy-4-propoxy[1,1′-biphenyl]-3-carboxamide;
303 N-[(R)-1-(Hydroxymethyl)-2-(1H-indol-3-yl)ethyl]-3′-methoxy-4-propoxy[1,1′-biphenyl]-3-carboxamide;
304 N-[(R)-1-(Hydroxymethyl)-2-(1H-indol-3-yl)ethyl]-N′-methyl-4-propoxy[1,1′-biphenyl]-3,3′-dicarboxamide;
305 4,3′,4′,5′-Tetramethoxybiphenyl-3-carboxylic acid [(R)-1-hydroxymethyl-2-(1H-indol-3-yl)ethyl]amide;
306 4,3′,4′-Trimethoxybiphenyl-3-carboxylic acid [(R)-1-hydroxymethyl-2-(1H-indol-3-yl)ethyl]amide;
307 3′-Fluoro-4,4′-dimethoxybiphenyl-3-carboxylic acid [(R)-2-hydroxy-1-(1H-indol-3-ylmethyl)ethyl]amide;
308 4,3′-Dimethoxybiphenyl-3-carboxylic acid [(R)-1-hydroxymethyl-2-(1H-indol-3-yl)ethyl]amide;
309 5-Benzo[1,3]dioxol-5-yl-N-[(R)-1-hydroxymethyl-2-(1H-indol-3-yl)ethyl]-2-methoxybenzamide;
310 3′,4′-Difluoro-4,5′-dimethoxybiphenyl-3-carboxylic acid [(R)-2-hydroxy-1-(1H-indol-3-ylmethyl)ethyl]amide;
311 4-Isopropoxy-3′-methoxybiphenyl-3-carboxylic acid [(R)-1-hydroxymethyl-2-(1H-indol-3-yl)ethyl]amide;
312 5-Benzo[1,3]dioxol-5-yl-N-[(R)-1-hydroxymethyl-2-(1H-indol-3-yl)ethyl]-2-isopropoxybenzamide;
313 4-Isopropoxy-3′,4′,5′-trimethoxybiphenyl-3-carboxylic acid [(R)-1-hydroxymethyl-2-(1H-indol-3-yl)ethyl]amide;
314 3′-Fluoro-4-isopropoxy-4′-methoxybiphenyl-3-carboxylic acid [(R)-2-hydroxy-1-(1H-indol-3-ylmethyl)ethyl]amide;
315 4-Isopropoxy-3′,4′-dimethoxybiphenyl-3-carboxylic acid [(R)-1-hydroxymethyl-2-(1H-indol-3-yl)ethyl]amide;
316 4-Isopropoxy-3′-methylbiphenyl-3-carboxylic acid [(R)-1-hydroxymethyl-2-(1H-indol-3-yl)ethyl]amide;
317 4′-Fluoro-4-isopropoxy-3′-methylbiphenyl-3-carboxylic acid [(R)-2-hydroxy-1-(1H-indol-3-ylmethyl)ethyl]amide;
318 3′,4′-Difluoro-4-isopropoxy-5′-methoxybiphenyl-3-carboxylic acid [(R)-2-hydroxy-1-(1H-indol-3-ylmethyl)ethyl]amide;
319 4,3′,4′,5′-Tetramethoxy-5-methylbiphenyl-3-carboxylic acid [(R)-1-hydroxymethyl-2-(1H-indol-3-yl)ethyl]amide;
320 4,3′,4′-Trimethoxy-5-methylbiphenyl-3-carboxylic acid [(R)-1-hydroxymethyl-2-(1H-indol-3-yl)ethyl]amide;
321 3′-Fluoro-4,4′-dimethoxy-5-methylbiphenyl-3-carboxylic acid [(R)-2-hydroxy-1-(1H-indol-3-ylmethyl)ethyl]amide;
322 5-Benzo[1,3]dioxol-5-yl-N-[(R)-1-hydroxymethyl-2-(1H-indol-3-yl)ethyl]-2-methoxy-3-methylbenzamide;
323 4,3′-Dimethoxy-5-methylbiphenyl-3-carboxylic acid [(R)-1-hydroxymethyl-2-(1H-indol-3-yl)ethyl]amide;
324 4-Methoxy-5,3′-dimethylbiphenyl-3-carboxylic acid [(R)-1-hydroxymethyl-2-(1H-indol-3-yl)ethyl]amide;
325 4′-Fluoro-4-methoxy-5,3′-dimethylbiphenyl-3-carboxylic acid [(R)-2-hydroxy-1-(1H-indol-3-ylmethyl)ethyl]amide;
326 3′,4′-Difluoro-4,5′-dimethoxy-5-methylbiphenyl-3-carboxylic acid [(R)-2-hydroxy-1-(1H-indol-3-ylmethyl)ethyl]amide;
327 3′-Hydroxy-4-isopropoxybiphenyl-3-carboxylic acid [(R)-1-hydroxymethyl-2-(1H-indol-3-yl)ethyl]amide;
328 3′,4′,5′-Trimethoxy-4-(3-methyl-but-2-enyloxy)biphenyl-3-carboxylic acid [(R)-1-hydroxymethyl-2-(1H-indol-3-yl)ethyl]amide;
329 3′-Butoxy-4-ethoxybiphenyl-3-carboxylic acid [(R)-1-hydroxymethyl-2-(1H-indol-3-yl)ethyl]amide;
330 4-Ethoxy-3′-isopropoxybiphenyl-3-carboxylic acid [(R)-1-hydroxymethyl-2-(1H-indol-3-yl)ethyl]amide;
331 N-[(R)-1-Hydroxymethyl-2-(1H-indol-3-yl)ethyl]-5-(7-methoxybenzofuran-2-yl)-2-propoxybenzamide;
332 6-Methoxy-2-(3,4,5-trimethoxyphenyl)quinoline-4-carboxylic acid [(R)-2-(6-chlor-1H-indol-3-yl)-1-hydroxymethylethyl]amide;
333 6-Methoxy-2-(3,4,5-trimethoxyphenyl)quinoline-4-carboxylic acid [(R)-1-hydroxymethyl-2-(2-methyl-1H-indol-3-yl)ethyl]amide;
334 6-Methoxy-2-(3,4,5-trimethoxyphenyl)quinoline-4-carboxylic acid [1-hydroxymethyl-2-(6-methyl-1H-indol-3-yl)ethyl]amide;
335 N-[(R)-1-(Hydroxymethyl)-2-(1-ethyl)-1H-indol-3-yl)ethyl]-6-methoxy-2-(3-methoxyphenyl)quinoline-4-carboxamide;
336 N-[(R)-1-(Hydroxymethyl)-2-(1-propyl-1H-indol-3-yl)ethyl]-6-methoxy-2-(3,4,5-trimethoxyphenyl)quinoline-4-carboxamide;
337 N-[(R)-1-(Hydroxymethyl)-2-(1-ethyl)-1H-indol-3-yl)ethyl]-2-(3,5-difluoro-4-methoxyphenyl)-6-methoxyquinoline-4-carboxamide;
338 N-[(R)-1-(Hydroxymethyl)-2-(1-isopropyl-1H-indol-3-yl)ethyl]-6-methoxy-2 (3-methoxyphenyl)-quinoline-4-carboxamide;
339 N-[(R)-1-(Hydroxymethyl)-2-(1-isopropyl-1H-indol-3-yl)ethyl]-2-(3,5-difluoro-4-methoxyphenyl)-6-methoxyquinoline-4-carboxamide;
340 N-[(R)-1-(Hydroxymethyl)-2-(1-isopropyl-1H-indol-3-yl)ethyl]-6-methoxy-2-(3,4,5-trimethoxyphenyl)quinoline-4-carboxamide;
341 N-[(R)-1-(Hydroxymethyl)-2-(1-ethyl)-1H-indol-3-yl)ethyl]-2-(3-fluoro-4-methoxyphenyl)-6-trifluoromethoxyquinoline-4-carboxamide;
342 N-[(R)-1-(Hydroxymethyl)-2-(1-ethyl)-1H-indol-3-yl)ethyl]-2-(7-methoxybenzofuran-2-yl)-6-trifluoromethoxyquinoline-4-carboxamide;
343 N-[(R)-1-(Hydroxymethyl)-2-(1-isopropyl-1H-indol-3-yl)ethyl]-2-(3-fluoro-4-methoxyphenyl)-6-trifluoromethoxyquinoline-4-carboxamide;
344 N-[(R)-1-(Hydroxymethyl)-2-(1-isopropyl-1H-indol-3-yl)ethyl]-2-(7-methoxybenzofuran-2-yl)-6-trifluoromethoxyquinoline-4-carboxamide;
345 N-[(R)-1-(Hydroxymethyl)-2-(1-n hexyl-1H-indol-3-yl)ethyl]-6-methoxy-2-(3,4,5-trimethoxyphenyl)quinoline-4-carboxamide;
346 N-[(R)-1-(Hydroxymethyl)-2-(1-ethyl)-1H-indol-3-yl)ethyl]-4-ethoxy-3′-methoxybiphenyl-3-carboxamide;
333 N-[(R)-1-(Hydroxymethyl)-2-(1-isopropyl)-1H-indol-3-yl)ethyl]-4-ethoxy-3′-methoxybiphenyl)-3-carboxamide;
347 N-[(R)-2-(1-Ethyl-1H-indol-3-yl)-1-(hydroxymethyl)ethyl]-3′-fluoro-4′-methoxy[1,1′-biphenyl]-3-carboxamide;
348 3′-Fluoro-N-[(R)-1-(hydroxymethyl)-2-(1-propyl-1H-indol-3-yl)ethyl]-4′-methoxy[1,1′-biphenyl]-3-carboxamide;
349 N-[(R)-2-(1-Butyl-1H-indol-3-yl)-1-(hydroxymethyl)ethyl]-3′-fluoro-4′-methoxy[1,1′-biphenyl]-3-carboxamide;
350 3′-Fluoro-N-[(R)-1-(hydroxymethyl)-2-[1-(3-methylbutyl)-1H-indol-3-yl]ethyl]-4′-methoxy[1,1′-biphenyl]-3-carboxamide;
351 3′-Fluoro-N-[(R)-1-(hydroxymethyl)-2-(1-pentyl-1H-indol-3-yl)ethyl]-4′-methoxy[1,1′-biphenyl]-3-carboxamide;
352 3′-Fluoro-N-[(R)-2-(1-hexyl-1H-indol-3-yl)-1-(hydroxymethyl)ethyl]-4′-methoxy[1,1′-biphenyl]-3-carboxamide;
353 4-Ethoxy-3′-methoxybiphenyl-3-carboxylic acid [2-(5,6-difluoro-1H-indol-3-yl)-1-hydroxymethylethyl]amide;
354 6-Methoxy-2-(3,4,5-trimethoxyphenyl)quinoline-4-carboxylic acid [2-(5,6-difluoro-1H-indol-3-yl)-1-hydroxymethylethyl]amide;
355 N-[(R)-1-(Hydroxymethyl)-2-(1-ethyl-5-fluoro-1H-indol-3-yl)ethyl]-6-methoxy-2-(3,4,5-trimethoxyphenyl)quinoline-4-carboxamide;
356 6-Methoxy-2-(3,4,5-trimethoxyphenyl)quinoline-4-carboxylic acid [2-(1-ethyl-5-fluoro-1H-indol-3-yl)-1-hydroxymethylethyl]amide;
357 6-(3,4,5-Trimethoxyphenyl)quinoline-8-carboxylic acid [(R)-1-hydroxymethyl-2-(1H-indol-3-yl)ethyl]amide;
358 3-(3,4,5-Trimethoxyphenyl)naphthalene-1-carboxylic acid [(R)-1-hydroxymethyl-2-(1H-indol-3-yl)ethyl]amide;
359 4-Methoxy-5-(3,4,5-trimethoxyphenyl)thiophene-3-carboxylic acid [(R)-2-hydroxy-1-(1H-indol-3-ylmethyl)ethyl]amide;
360 6-(3,4,5-Trimethoxyphenyl)-1H-benzoimidazol-4-carboxylic acid [(R)-2-hydroxy-1-(1H-indol-3-ylmethyl)ethyl]amide;
361 2-(3,4,5-Trimethoxyphenyl)thiazol-4-carboxylic acid [(R)-2-hydroxy-1-(1H-indol-3-ylmethyl)ethyl]amide;
362 5-(3,4,5-Trimethoxyphenyl)thiophene-2-carboxylic acid [(R)-2-hydroxy-1-(1H-indol-3-ylmethyl)ethyl]amide;
363 5-(3,4,5-Trimethoxyphenyl)benzo[b]thiophene-2-carboxylic acid [(R)-2-hydroxy-1-(1H-indol-3-ylmethyl)ethyl]amide;
364 2-(3-Fluoro-4-methoxyphenyl)-N-[(R)-2-hydroxy-1-(1H-indol-3-ylmethyl)ethyl]-6-methylisonicotinamide;
365 2-(3-Fluoro-4-methoxyphenyl)-6-methylpyrimidine-4-carboxylic acid [(R)-2-hydroxy-1-(1H-indol-3-ylmethyl)ethyl]amide;
366 6-(4-Methoxyphenyl)pyrimidine-4-carboxylic acid [(R)-1-hydroxymethyl-2-(1H-indol-3-yl)ethyl]amide;
367 2-(3-Fluoro-4-methoxyphenyl)-6-methoxyquinazoline-4-carboxylic acid [(R)-2-hydroxy-1-(1H-indol-3-ylmethyl)ethyl]amide;
368 2-(3-Fluoro-4-methoxyphenyl)-6-iodoquinazoline-4-carboxylic acid [(R)-2-hydroxy-1-(1H-indol-3-ylmethyl)ethyl]amide;
369 2-(4-Methoxyphenyl)quinazoline-4-carboxylic acid [(R)-1-hydroxymethyl-2-(1H-indol-3-yl)ethyl]amide;
370 2-(3-Fluoro-4-methoxyphenyl)-6-methoxyquinazoline-4-carboxylic acid [(R)-1-(1-ethyl-1H-indol-3-ylmethyl)-2-hydroxyethyl]amide;
371 2-(3,4,5-Trimethoxyphenyl)quinoline-4-carboxylic acid [(R)-2-hydroxy-1-(1H-indol-3-ylmethyl)-2-methylpropyl]amide;
372 6-Methoxy-2-(3,4,5-trimethoxyphenyl)quinoline-4-carboxylic acid [(R)-2-hydroxy-1-(1H-indol-3-ylmethyl)-2-methylpropyl]amide;
373 6-(4-Hydroxybut-1-ynyl)-2-(3,4,5-trimethoxyphenyl)quinoline-4-carboxylic acid [(R)-1-hydroxymethyl-2-(1H-indol-3-yl)ethyl]amide;
374 6-(5-Hydroxypent-1-ynyl)-2-(3,4,5-trimethoxyphenyl)quinoline-4-carboxylic acid [(R)-1-hydroxymethyl-2-(1H-indol-3-yl)ethyl]amide;
375 6-(3-Hydroxyprop-1-ynyl)-2-(3,4,5-trimethoxyphenyl)quinoline-4-carboxylic acid [(R)-1-hydroxymethyl-2-(1H-indol-3-yl)ethyl]amide;
376 6-(3-Methoxyprop-1-ynyl)-2-(3,4,5-trimethoxyphenyl)quinoline-4-carboxylic acid [(R)-1-hydroxymethyl-2-(1H-indol-3-yl)ethyl]amide;
377 5-(4-Hydroxybut-1-ynyl)-3′,4′,5′-trimethoxybiphenyl-3-carboxylic acid [(R)-1-hydroxymethyl-2-(1H-indol-3-yl)ethyl]amide;
378 5-(3-Hydroxyprop-1-ynyl)-3′,4′,5′-trimethoxybiphenyl-3-carboxylic acid [(R)-1-hydroxymethyl-2-(1H-indol-3-yl)ethyl]amide;
379 5-(5-Hydroxypent-1-ynyl)-3′,4′,5′-trimethoxybiphenyl-3-carboxylic acid [(R)-1-hydroxymethyl-2-(1H-indol-3-yl)ethyl]amide;
380 3′,4′,5′-Trimethoxy-5-(3-methoxyprop-1-ynyl)biphenyl-3-carboxylic acid [(R)-1-hydroxymethyl-2-(1H-indol-3-yl)ethyl]amide;
381 3′,4′-Dimethoxy-5-(3-methoxyprop-1-ynyl)biphenyl-3-carboxylic acid [(R)-1-hydroxymethyl-2-(1H-indol-3-yl)ethyl]amide;
382 5-(3-Hydroxyprop-1-ynyl)-3′,4′-dimethoxybiphenyl-3-carboxylic acid [(R)-1-hydroxymethyl-2-(1H-indol-3-yl)ethyl]amide;
383 3′,4′,5′-Trimethoxy-5-(4-methoxyphenylethynyl)biphenyl-3-carboxylic acid [(R)-1-hydroxymethyl-2-(1H-indol-3-yl)ethyl]amide;
384 3′,4′,5′-Trimethoxy-5-((Z)-3-methoxypropenyl)biphenyl-3-carboxylic acid [(R)-1-hydroxymethyl-2-(1H-indol-3-yl)ethyl]amide;
385 5-((Z)-4-Hydroxybut-1-enyl)-3′,4′,5′-trimethoxybiphenyl-3-carboxylic acid [(R)-1-hydroxymethyl-2-(1H-indol-3-yl)ethyl]amide;
386 5-((Z)-3-Hydroxypropenyl)-3′,4′,5′-trimethoxybiphenyl-3-carboxylic acid [(R)-1-hydroxymethyl-2-(1H-indol-3-yl)ethyl]amide;
387 5-((Z)-5-Hydroxypent-1-enyl)-3′,4′,5′-trimethoxybiphenyl-3-carboxylic acid [(R)-1-hydroxymethyl-2-(1H-indol-3-yl)ethyl]amide;
388 6-(5-Hydroxypentyl)-2-(3,4,5-trimethoxyphenyl)quinoline-4-carboxylic acid [(R)-1-hydroxymethyl-2-(1H-indol-3-yl)ethyl]amide;
389 6-(4-Hydroxybutyl)-2-(3,4,5-trimethoxyphenyl)quinoline-4-carboxylic acid [(R)-1-hydroxymethyl-2-(1H-indol-3-yl)ethyl]amide;
390 6-(3-Hydroxypropyl)-2-(3,4,5-trimethoxyphenyl)quinoline-4-carboxylic acid [(R)-1-hydroxymethyl-2-(1H-indol-3-yl)ethyl]amide;
391 6-(3-Methoxypropyl)-2-(3,4,5-trimethoxyphenyl)quinoline-4-carboxylic acid [(R)-1-hydroxymethyl-2-(1H-indol-3-yl)ethyl]amide;
392 3′,4′,5′-Trimethoxy-4-(3-methoxypropyl)biphenyl-3-carboxylic acid [(R)-1-hydroxymethyl-2-(1H-indol-3-yl)ethyl]amide;
393 3′,4′,5′-Trimethoxy-5-(3-methoxypropyl)biphenyl-3-carboxylic acid [(R)-1-hydroxymethyl-2-(1H-indol-3-yl)ethyl]amide;
394 3′,4′-Dimethoxy-5-(3-methoxypropyl)biphenyl-3-carboxylic acid [(R)-1-hydroxymethyl-2-(1H-indol-3-yl)ethyl]amide;
395 5-(3-Hydroxypropyl)-3′,4′-dimethoxybiphenyl-3-carboxylic acid [(R)-1-hydroxymethyl-2-(1H-indol-3-yl)ethyl]amide;
396 5-(5-Hydroxypentyl)-3′,4′,5′-trimethoxybiphenyl-3-carboxylic acid [(R)-1-hydroxymethyl-2-(1H-indol-3-yl)ethyl]amide;
397 5-(3-Hydroxypropyl)-3′,4′,5′-trimethoxybiphenyl-3-carboxylic acid [(R)-1-hydroxymethyl-2-(1H-indol-3-yl)ethyl]amide;
398 5-(4-Hydroxybutyl)-3′,4′,5′-trimethoxybiphenyl-3-carboxylic acid [(R)-1-hydroxymethyl-2-(1H-indol-3-yl)ethyl]amide;
399 3′,4′,5′-Trimethoxy-5-[2-(4-methoxyphenyl)ethyl]-biphenyl-3-carboxylic acid [(R)-1-hydroxymethyl-2-(1H-indol-3-yl)ethyl]amide;
400 N-[(R)-2-[1-(2-Cyanoethyl)-1H-indol-3-yl]-1-(hydroxymethyl)ethyl]-3′-fluoro-4′-methoxy[1,1′-biphenyl]-3-carboxamide;
401 3′-Fluoro-N-[(R)-2-(1-heptyl-1H-indol-3-yl)-1-(hydroxymethyl)ethyl]-4′-methoxy[1,1′-biphenyl]-3-carboxamide;
402 N-[(R)-2-[1-(4-Cyanobutyl)-1H-indol-3-yl]-1-(hydroxymethyl)ethyl]-3′-fluoro-4′-methoxy[1,1′-biphenyl]-3-carboxamide;
403 3′-Fluoro-N-[(R)-1-(hydroxymethyl)-2-[1-(3-phenoxypropyl)-1H-indol-3-yl]ethyl]-4′-methoxy[1,1′-biphenyl]-3-carboxamide;
404 3′-Fluoro-N-[(R)-1-(hydroxymethyl)-2-[1-(2-methoxyethyl)-1H-indol-3-yl]ethyl]-4′-methoxy[1,1′-biphenyl]-3-carboxamide;
405 N-[(R)-2-[1-(3-Cyanopropyl)-1H-indol-3-yl]-1-(hydroxymethyl)ethyl]-3′-fluoro-4′-methoxy[1,1′-biphenyl]-3-carboxamide;
406 4-Ethoxy-3′-methoxybiphenyl-3-carboxylic acid [(R)-2-(1-cyanomethyl-1H-indol-3-yl)-1-hydroxymethylethyl]amide;
407 6-Methoxy-2-(3,4,5-trimethoxyphenyl)quinoline-4-carboxylic acid [(R)-2-(1-cyanomethyl-1H-indol-3-yl)-1-hydroxymethylethyl]amide;
408 6-Methoxy-2-(3,4,5-trimethoxyphenyl)quinoline-4-carboxylic acid {(R)-2-[1-(4-cyanobutyl)-1H-indol-3-yl]-1-hydroxymethylethyl}amide;
409 4-Hydroxy-3′,4′,5′-trimethoxybiphenyl-3-carboxylic acid [(R)-1-hydroxymethyl-2-(1H-indol-3-yl)ethyl]amide;
410 4-(3-Cyanopropoxy)-3′,4′,5′-trimethoxybiphenyl-3-carboxylic acid [(R)-1-hydroxymethyl-2-(1H-indol-3-yl)ethyl]amide;
411 4-Cyclopentyloxy-3′-fluoro-4′-methoxybiphenyl-3-carboxylic acid [(R)-2-hydroxy-1-(1H-indol-3-ylmethyl)ethyl]amide;
412 4-Cyclopentyloxy-3′-methylbiphenyl-3-carboxylic acid [(R)-1-hydroxymethyl-2-(1H-indol-3-yl)ethyl]amide;
413 3′-(1-Butyl-3-methylureido)-4-cyclopentyloxybiphenyl-3-carboxylic acid [(R)-1-hydroxymethyl-2-(1H-indol-3-yl)ethyl]amide;
414 4-Cyclopentyloxy-4′-fluoro-3′-methylbiphenyl-3-carboxylic acid [(R)-2-hydroxy-1-(1H-indol-3-ylmethyl)ethyl]amide;
415 4-Cyclopentyloxy-3′-methoxybiphenyl-3-carboxylic acid [(R)-1-hydroxymethyl-2-(1H-indol-3-yl)ethyl]amide;
416 4-Cyclopentyloxy-3′,4′-dimethoxybiphenyl-3-carboxylic acid [(R)-1-hydroxymethyl-2-(1H-indol-3-yl)ethyl]amide;
417 5-Benzo[1,3]dioxol-5-yl-2-cyclopentyloxy-N-[(R)-1-hydroxymethyl-2-(1H-indol-3-yl)ethyl]benzamide;
418 4-Cyclopentyloxy-3′,4′,5′-trimethoxybiphenyl-3-carboxylic acid [(R)-1-hydroxymethyl-2-(1H-indol-3-yl)ethyl]amide;
419 4-Cyclopentyloxy-3′,4′-difluoro-5′-methoxybiphenyl-3-carboxylic acid [(R)-2-hydroxy-1-(1H-indol-3-ylmethyl)ethyl]amide;
420 3′-[Butyl[(1,1-dimethylethoxy)carbonyl]amino]-4-ethoxy-N-[(R)-1-(hydroxymethyl)-2-(1H-indol-3-yl)ethyl][1,1′-biphenyl]-3-carboxamide;
421 3′-(Butylamino)-4-ethoxy-N-[(R)-1-(hydroxymethyl)-2-(1H-indol-3-yl)ethyl][1,1′-biphenyl]-3-carboxamide;
422 3′-[Butyl[(methylamino)carbonyl]amino]-4-ethoxy-N-[(R)-1-(hydroxymethyl)-2-(1H-indol-3-yl)ethyl][1,1′-biphenyl]-3-carboxamide;
423 3′-[Butyl[(1,1-dimethylethoxy)carbonyl]amino]-N-[(R)-1-(hydroxymethyl)-2-(1H-indol-3-yl)ethyl]-4-propoxy[1,1′-biphenyl]-3-carboxamide;
424 3′-(1-Butyl-3-methylureido)-4-methoxybiphenyl-3-carboxylic acid [(R)-1-hydroxymethyl-2-(1H-indol-3-yl)ethyl]amide;
425 3′-(1-Butyl-3-methylureido)-4-methoxy-5-methylbiphenyl-3-carboxylic acid [(R)-1-hydroxymethyl-2-(1H-indol-3-yl)ethyl]amide;
426 3′-(1-Butyl-3-methylureido)-4-isopropoxybiphenyl-3-carboxylic acid [(R)-1-hydroxymethyl-2-(1H-indol-3-yl)ethyl]amide;
427 3′-(2-Dimethylaminoethoxy)-4-ethoxybiphenyl-3-carboxylic acid [(R)-1-hydroxymethyl-2-(1H-indol-3-yl)ethyl]amide;
428 4′-Ethoxy-3′-[(R)-1-hydroxymethyl-2-(1H-indol-3-yl)ethylcarbamoyl]-biphenyl-3-carboxylic acid methyl ester;
429 4-Ethoxy-[1,1′;3′,1″]terphenyl-3-carboxylic acid [1-hydroxymethyl-2-(1H-indol-3-yl)ethyl]amide;
430 3′-Acetyl-4-ethoxybiphenyl-3-carboxylic acid [(R)-1-hydroxymethyl-2-(1H-indol-3-yl)ethyl]amide;
431 4-Ethoxy-3′-pyrrolidin-1-ylbiphenyl-3-carboxylic acid [(R)-1-hydroxymethyl-2-(1H-indol-3-yl)ethyl]amide;
432 4′-Cyanomethyl-4-ethoxybiphenyl-3-carboxylic acid [(R)-1-hydroxymethyl-2-(1H-indol-3-yl)ethyl]amide;
433 4′-Dimethylamino-4-propoxybiphenyl-3-carboxylic acid [(R)-1-hydroxymethyl-2-(1H-indol-3-yl)ethyl]amide;
434 4′-Hydroxymethyl-4-propoxybiphenyl-3-carboxylic acid [(R)-1-hydroxymethyl-2-(1H-indol-3-yl)ethyl]amide;
435 4-Propoxybiphenyl-3,4′-dicarboxylic acid 4′-diethylamide 3-{[(R)-1-hydroxymethyl-2-(1H-indol-3-yl)ethyl]amide};
436 3′-[(R)-1-Hydroxymethyl-2-(1H-indol-3-yl)ethylcarbamoyl]-4′-propoxybiphenyl-4-carboxylic acid;
437 4′-Acetyl-4-propoxybiphenyl-3-carboxylic acid [(R)-1-hydroxymethyl-2-(1H-indol-3-yl)ethyl]amide;
438 4′-Ethanesulphonyl-4-propoxybiphenyl-3-carboxylic acid [(R)-2-hydroxy-1-(1H-indol-3-ylmethyl)ethyl]amide;
439 3′-Cyanomethyl-4-propoxybiphenyl-3-carboxylic acid [(R)-1-hydroxymethyl-2-(1H-indol-3-yl)ethyl]amide;
440 3′-Methanesulphonylamino-4-propoxybiphenyl-3-carboxylic acid [(R)-2-hydroxy-1-(1H-indol-3-ylmethyl)ethyl]amide;
441 3′-Cyclopropylmethoxy-4-propoxybiphenyl-3-carboxylic acid [(R)-1-hydroxymethyl-2-(1H-indol-3-yl)ethyl]amide;
442 3′-Methanesulphonyl-4-propoxybiphenyl-3-carboxylic acid [(R)-2-hydroxy-1-(1H-indol-3-ylmethyl)ethyl]amide;
443 4-Propoxybiphenyl-3,3′-dicarboxylic acid 3′-[(2-dimethylaminoethyl)-amide]3-{[(R)-1-hydroxymethyl-2-(1H-indol-3-yl)ethyl]amide};
444 3′-[(R)-1-Hydroxymethyl-2-(1H-indol-3-yl)ethylcarbamoyl]-3-methoxy-4′-propoxybiphenyl-4-carboxylic acid methyl ester;
445 3′-Chloro-4-propoxybiphenyl-3,4′-dicarboxylic acid 4′-amide 3-{[(R)-2-hydroxy-1-(1H-indol-3-ylmethyl)ethyl]amide};
446 3′-Dimethylsulphamoyl-4-propoxybiphenyl-3-carboxylic acid [(R)-2-hydroxy-1-(1H-indol-3-ylmethyl)ethyl]amide;
447 4′-(Propane-2-sulphonyl)-4-propoxybiphenyl-3-carboxylic acid [(R)-2-hydroxy-1-(1H-indol-3-ylmethyl)ethyl]amide;
448 4′-Methylsulphamoyl-4-propoxybiphenyl-3-carboxylic acid [(R)-2-hydroxy-1-(1H-indol-3-ylmethyl)ethyl]amide;
449 4′-Dimethylsulphamoyl-4-propoxybiphenyl-3-carboxylic acid [(R)-2-hydroxy-1-(1H-indol-3-ylmethyl)ethyl]amide;
450 4-Propoxybiphenyl-3,4′-dicarboxylic acid 4′-amide 3-{[(R)-1-hydroxymethyl-2-(1H-indol-3-yl)ethyl]amide};
451 3′-Methylsulphamoyl-4-propoxybiphenyl-3-carboxylic acid [(R)-2-hydroxy-1-(1H-indol-3-ylmethyl)ethyl]amide;
452 3′-Methanesulphonyl-4-propoxybiphenyl-3-carboxylic acid [(R)-2-hydroxy-1-(1-methyl-1H-indol-3-ylmethyl)ethyl]amide;
453 3′-[(R)-1-Hydroxymethyl-2-(1-methyl-1H-indol-3-yl)ethylcarbamoyl]-3-methoxy-4′-propoxybiphenyl-4-carboxylic acid methyl ester;
454 4-Propoxybiphenyl-3,4′-dicarboxylic acid 4′-[(2-dimethylaminoethyl)amide]3-{[(R)-1-hydroxymethyl-2-(1-methyl-1H-indol-3-yl)ethyl]amide};
455 3′-[2-(5-Fluoro-1H-indol-3-yl)-1-hydroxymethylethylcarbamoyl]-4′-propoxybiphenyl-4-carboxylic acid;
456 3′-Methanesulphonylamino-4-propoxybiphenyl-3-carboxylic acid [2-(5-fluoro-1H-indol-3-yl)-1-hydroxymethylethyl]amide;
457 3′-Methanesulphonyl-4-propoxybiphenyl-3-carboxylic acid [2-(5-fluoro-1H-indol-3-yl)-1-hydroxymethylethyl]amide;
458 4-Propoxybiphenyl-3,3′-dicarboxylic acid 3′-[(2-dimethylaminoethyl)amide]3-{[2-(5-fluoro-1H-indol-3-yl)-1-hydroxymethylethyl]amide};
459 3′-Chloro-4-propoxybiphenyl-3,4′-dicarboxylic acid 4′-amide 3-{[2-(5-fluoro-1H-indol-3-yl)-1-hydroxymethylethyl]amide};
460 3′-Dimethylsulphamoyl-4-propoxybiphenyl-3-carboxylic acid [2-(5-fluoro-1H-indol-3-yl)-1-hydroxymethylethyl]amide;
461 4′-(Propane-2-sulphonyl)-4-propoxybiphenyl-3-carboxylic acid [2-(5-fluoro-1H-indol-3-yl)-1-hydroxymethylethyl]amide;
462 4′-Dimethylsulphamoyl-4-propoxybiphenyl-3-carboxylic acid [2-(5-fluoro-1H-indol-3-yl)-1-hydroxymethylethyl]amide;
463 4-Propoxybiphenyl-3,4′-dicarboxylic acid 4′-diethylamide 3-{[2-(5-fluoro-1H-indol-3-yl)-1-hydroxymethylethyl]amide};
464 3′-Methylsulphamoyl-4-propoxybiphenyl-3-carboxylic acid [2-(5-fluoro-1H-indol-3-yl)-1-hydroxymethylethyl]amide;
465 3′-Acetyl-4-propoxybiphenyl-3-carboxylic acid [(R)-1-hydroxymethyl-2-(1-methyl-1H-indol-3-yl)ethyl]amide;
466 4-Propoxy-[1,1′;3′,1″]terphenyl-3-carboxylic acid [(R)-1-hydroxymethyl-2-(1-methyl-1H-indol-3-yl)ethyl]amide;
467 3′-Cyanomethyl-4-propoxybiphenyl-3-carboxylic acid [(R)-1-hydroxymethyl-2-(1-methyl-1H-indol-3-yl)ethyl]amide;
468 3′-Methanesulphonylamino-4-propoxybiphenyl-3-carboxylic acid [(R)-2-hydroxy-1-(1-methyl-1H-indol-3-ylmethyl)ethyl]amide;
469 4′-Cyanomethyl-4-propoxybiphenyl-3-carboxylic acid [(R)-1-hydroxymethyl-2-(1-methyl-1H-indol-3-yl)ethyl]amide;
470 4-Propoxybiphenyl-3,3′-dicarboxylic acid 3′-[(2-dimethylaminoethyl)amide]3-{[(R)-1-hydroxymethyl-2-(1-methyl-1H-indol-3-yl)ethyl]amide};
471 4-Fluoro-3′-[(R)-2-hydroxy-1-(1-methyl-1H-indol-3-ylmethyl)ethylcarbamoyl]-4′-propoxybiphenyl-3-carboxylic acid;
472 3′-Chloro-4-propoxybiphenyl-3,4′-dicarboxylic acid 4′-amide 3-{[(R)-2-hydroxy-1-(1-methyl-1H-indol-3-ylmethyl)ethyl]amide};
473 4-Propoxybiphenyl-3,4′-dicarboxylic acid 4′-diethylamide 3-{[(R)-1-hydroxymethyl-2-(1-methyl-1H-indol-3-yl)ethyl]amide};
474 4′-Dimethylamino-4-propoxybiphenyl-3-carboxylic acid [2-(5-fluoro-1H-indol-3-yl)-1-hydroxymethylethyl]amide;
475 4′-Acetyl-4-propoxybiphenyl-3-carboxylic acid [2-(5-fluoro-1H-indol-3-yl)-1-hydroxymethylethyl]amide;
476 3′-Acetyl-4-propoxybiphenyl-3-carboxylic acid [2-(5-fluoro-1H-indol-3-yl)-1-hydroxymethylethyl]amide;
477 4-Propoxy-[1,1′;3′,1″]terphenyl-3-carboxylic acid [2-(5-fluoro-1H-indol-3-yl)-1-hydroxymethylethyl]amide;
478 3′-[2-(5-Fluoro-1H-indol-3-yl)-1-hydroxymethylethylcarbamoyl]-3-methoxy-4′-propoxybiphenyl-4-carboxylic acid methyl ester;
480 4-Propoxybiphenyl-3,4′-dicarboxylic acid 4′-amide 3-{[2-(5-fluoro-1H-indol-3-yl)-1-hydroxymethylethyl]amide};
481 4-Ethoxy-4′-methoxymethylbiphenyl-3-carboxylic acid [(R)-1-hydroxymethyl-2-(1H-indol-3-yl)ethyl]amide;
482 4-Ethoxybiphenyl-3,3′-dicarboxylic acid 3′-amide 3-{[(R)-1-hydroxymethyl-2-(1H-indol-3-yl)ethyl]amide};
483 4′-Ethanesulphonyl-4-ethoxybiphenyl-3-carboxylic acid [(R)-2-hydroxy-1-(1H-indol-3-ylmethyl)ethyl]amide;
484 4-Ethoxy-4′-(4-methylpiperazine-1-carbonyl)biphenyl-3-carboxylic acid [(R)-1-hydroxymethyl-2-(1H-indol-3-yl)ethyl]amide;
3′-Cyclopropylmethoxy-4-ethoxybiphenyl-3-carboxylic acid [(R)-1-hydroxymethyl-2-(1H-indol-3-yl)ethyl]amide;
3′-[(R)-1-Hydroxymethyl-2-(1H-indol-3-yl)ethylcarbamoyl]biphenyl-2-carboxylic acid methyl ester.

The present invention also relates to a process for preparing the compounds according to the invention. Compounds of the general formula I or Ia can be prepared as shown in Scheme 1 by an amide-formation reaction between the tryptophanol derivative VI or VIa and the carboxylic acid VII. Reagents suitable for this purpose are all suitable peptide-coupling reagents which are known to the skilled person and which convert the carboxylic acid, where appropriate in the presence of a base, into an intermediate active ester, for example PyBOP ([(1H-benzotriazol-1-yl)oxy]tris(pyrrolidin-1-yl)phosphonium hexafluorophosphate), HATU (2-(7-aza-1H-benzotriazol-1-yl)-1,1,3,3-tetramethyluronium hexafluorophosphate), HBTU (2-(1H-benzotriazol-1-yl)-1,1,3,3-tetramethyluronium hexafluorophosphate), EDC (N-[3-(dimethylamino)propyl]-N′-ethylcarbodiimide hydrochloride)/HOBt (1-hydroxy-1H-benzotriazole). It is possible as alternative for the carboxylic acid to be converted, where appropriate in the presence of a base, into the carbonyl chloride and reacted with the tryptophanol VI or VIa to give the product of the general formula I or Ia.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding U.S. Provisional Application Ser. No. 60/706,743, filed Aug. 10, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

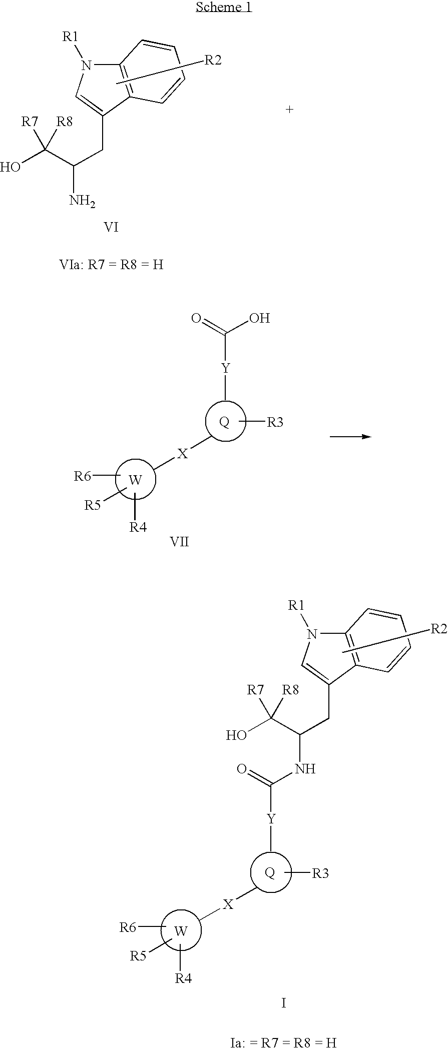

Compounds of general formulae II, IIa, III and IIIa can be prepared as shown in Scheme 2 by an amide-formation reaction between the tryptophanol derivative VI or VIa and the appropriate carboxylic acid VIII or IX. Reagents suitable for this purpose are all known peptide-coupling reagents which convert the carboxylic acid, where appropriate in the presence of a base, into an intermediate active ester, for example PyBOP ([(1H-benzotriazol-1-yl)oxy]tris(pyrrolidin-1-yl)phosphonium hexafluorophosphate), HATU (2-(7-aza-1H-benzotriazol-1-yl)-1,1,3,3-tetramethyluronium hexafluorophosphate), HBTU (2-(1H-benzotriazol-1-yl)-1,1,3,3-tetramethyluronium hexafluorophosphate), EDC (N-[3-(dimethylamino)propyl]-N′-ethylcarbodiimide hydrochloride)/HOBt (1-hydroxy-1H-benzotriazole). It is possible as alternative for the carboxylic acid to be converted, where appropriate in the presence of a base, into the carbonyl chloride and reacted with the tryptophanol VI or VIa to give the product of the general formula II, IIa, III or IIIa.

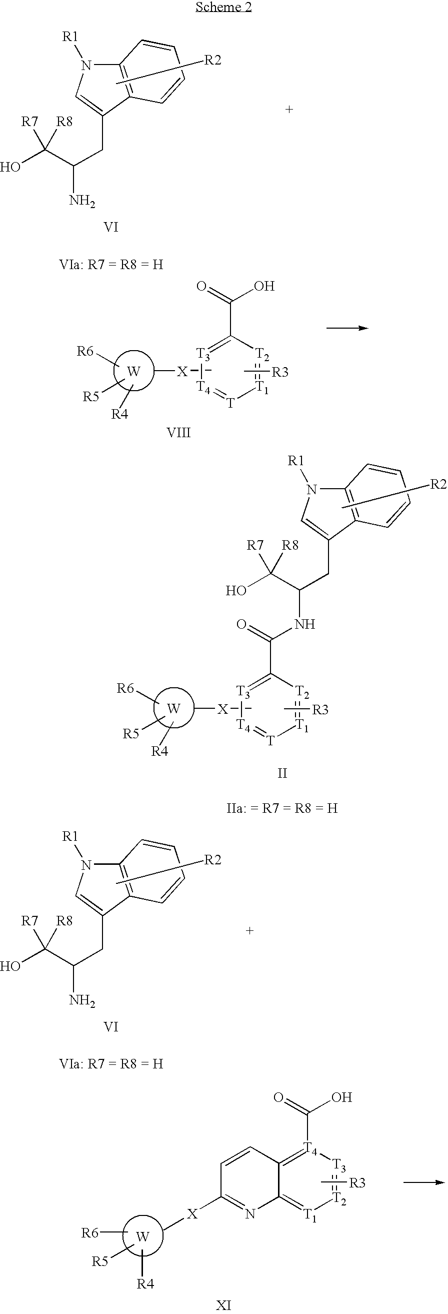

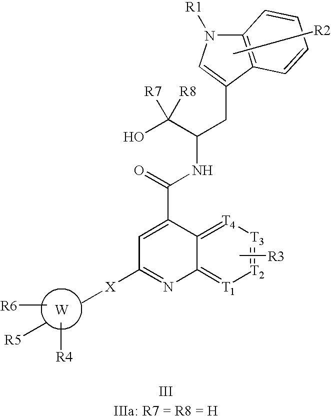

Compounds of general formulae IV, IVa, V and Va can be prepared as shown in Scheme 3 by an amide-formation reaction between the tryptophanol derivative VI or VIa and the appropriate carboxylic acid X or XI. Reagents suitable for this purpose are all suitable peptide-coupling reagents which are known to the skilled person and which convert the carboxylic acid, where appropriate in the presence of a base, into an intermediate active ester, for example PyBOP ([(1H-benzotriazol-1-yl)oxy]tris(pyrrolidin-1-yl)phosphonium hexafluorophosphate), HATU (2-(7-aza-1H-benzotriazol-1-yl)-1,1,3,3-tetramethyluronium hexafluorophosphate), HBTU (2-(1H-benzotriazol-1-yl)-1,1,3,3-tetramethyluronium hexafluorophosphate), EDC (N-[3-(dimethylamino)propyl]-N′-ethylcarbodiimide hydrochloride)/HOBt (1-hydroxy-1H-benzotriazole). It is possible as alternative for the carboxylic acid to be converted, where appropriate in the presence of a base, into the carbonyl chloride and reacted with the tryptophanol VI or VIa to give the product of the general formula IV, IVa, V or Va.

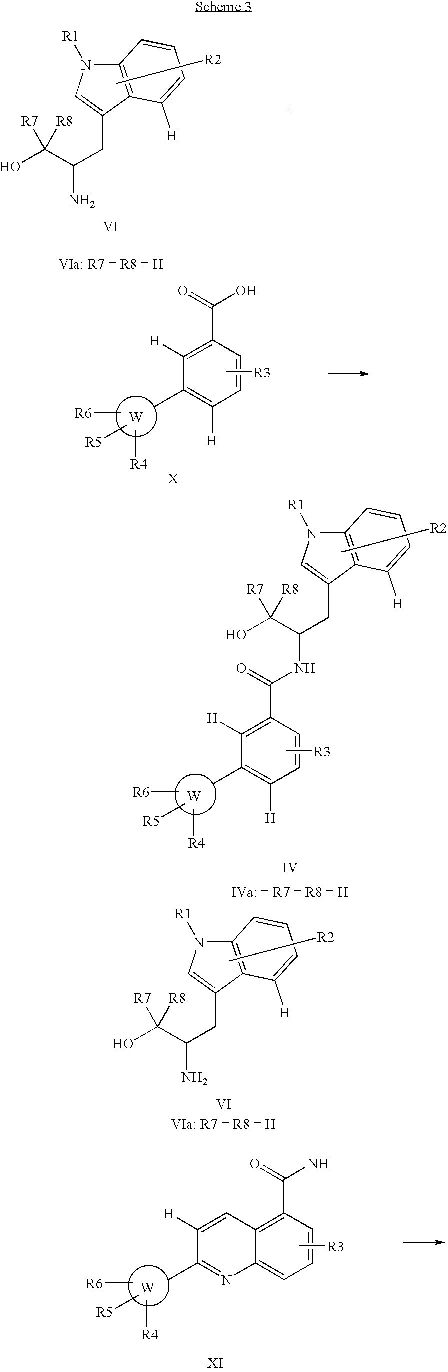

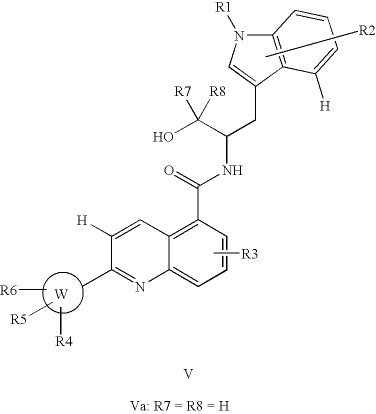

The present invention further relates to the carboxylic acids of the formulae VII, VIII, IX, X and XI as intermediates of the process according to the invention for preparing the compounds according to the invention, namely:
2-(4-Chloro-3-methylphenyl)quinoline-4-carboxylic acid;
6-Methoxy-2-(3,4,5-trimethoxyphenyl)quinoline-4-carboxylic acid;
6-Methoxy-2-(2,3,4-trimethoxyphenyl)quinoline-4-carboxylic acid;
6-Fluoro-2-(3,4,5-trimethoxyphenyl)quinoline-4-carboxylic acid;
6-Iodo-2-(3,4,5-trimethoxyphenyl)quinoline-4-carboxylic acid;
6-Nitro-2-(3,4,5-trimethoxyphenyl)quinoline-4-carboxylic acid;
2-[4-(Trifluoromethoxy)phenyl]quinoline-4-carboxylic acid;
2-(3,5-dimethoxyphenyl)quinoline-4-carboxylic acid;
2-[(E)-2-(3,4-dimethoxyphenyl)ethenyl]-6-methoxyquinoline-4-carboxylic acid;
2′,3′,4′-Trimethoxy[1,1′-biphenyl]-3-carboxylic acid;
3′,4′,5′-Trimethoxy[1,1′-biphenyl]-4-carboxylic acid;
3′,4′,5′-Trimethoxy-2-methyl[1,1′-biphenyl]-4-carboxylic acid;
2′,3′,4′-Trimethoxy-6-methyl[1,1′-biphenyl]-3-carboxylic acid;
2′,3′,4′-Trimethoxy[1,1′-biphenyl]-4-carboxylic acid;
2′,3′,4′-Trimethoxy-2-methyl[1,1′-biphenyl]-4-carboxylic acid;
3′,4,4′,5′-Tetramethoxy[1,1′-biphenyl]-4-carboxylic acid;
4′-(Hydroxymethyl)-6-methyl[1,1′-biphenyl]-3-carboxylic acid;
4′-(Hydroxymethyl)-2-methyl[1,1′-biphenyl]-4-carboxylic acid;
4-methoxy-3′-(1-methylethyl)[1,1′-biphenyl]-2-carboxylic acid;
3′-(1-methylethyl)[1,1′-biphenyl]-3-carboxylic acid;
6-methyl-3′-(1-methylethyl)[1,1′-biphenyl]-3-carboxylic acid;
3′-(1-methylethyl)[1,1′-biphenyl]-4-carboxylic acid;
2-methyl-3′-(1-methylethyl)[1,1′-biphenyl]-4-carboxylic acid;
4′-(Hydroxymethyl)-4-methoxy[1,1′-biphenyl]-2-carboxylic acid;
3′,4′,5′-Trifluoro[1,1′-biphenyl]-2-carboxylic acid;
3′,4′,5′-Trifluoro[1,1′-biphenyl]-3-carboxylic acid;
3′,4′,5′-Trifluoro-6-methyl[1,1′-biphenyl]-3-carboxylic acid;
3′,4′,5′-Trifluoro[1,1′-biphenyl]-4-carboxylic acid;
3′,4′,5′-Trifluoro-2-methyl[1,1′-biphenyl]-4-carboxylic acid;
2′,4,5′-Trimethoxy[1,1′-biphenyl]-2-carboxylic acid;
2′,4,5′-Trimethoxy[1,1′-biphenyl]-2-carboxylic acid;
2′,5′-dimethoxy[1,1′-biphenyl]-4-carboxylic acid;
2′,5′-dimethoxy-2-methyl[1,1′-biphenyl]-4-carboxylic acid;
3′,4,4′-Trimethoxy[1,1′-biphenyl]-2-carboxylic acid;
3′,4′-dimethoxy-6-methyl[1,1′-biphenyl]-2-carboxylic acid;
3′,4′-dimethoxy-2-methyl[1,1′-biphenyl]-4-carboxylic acid;
3′-Fluoro-4′-methoxy[1,1′-biphenyl]-2-carboxylic acid;
3′-Fluoro-4,4′-dimethoxy[1,1′-biphenyl]-2-carboxylic acid;
3′-Fluoro-4′-methoxy[1,1′-biphenyl]-3-carboxylic acid;
3′-Fluoro-4′-methoxy-6-methyl[1,1′-biphenyl]-3-carboxylic acid;
3′-Fluoro-4′-methoxy-2-methyl[1,1′-biphenyl]-4-carboxylic acid;
3′,4′-dimethoxy[1,1′-biphenyl]-2-carboxylic acid;
3′-(1-methylethyl)[1,1′-biphenyl]-2-carboxylic acid;
2′,5′-dimethoxy[1,1′-biphenyl]-3-carboxylic acid;
3′,4′,5′-Trifluoro-4-methoxy[1,1′-biphenyl]-2-carboxylic acid;
3-(Benzofuran-2-yl)benzoic acid;
3-(5-methoxybenzofuran-2-yl)benzoic acid;
2-[(3,4,5-Trimethoxyphenyl)methoxy]phenylpropanoic acid;
4-[[(3,4,5-Trimethoxyphenyl)methoxy]methyl]benzoic acid;
3-[(3,4,5-Trimethoxyphenyl)methoxy]thiophene-2-carboxylic acid;
4-[(3,4,5-Trimethoxyphenyl)methoxy]phenylacetic acid;
3-[3-((3,4,5-Trimethoxyphenyl)methoxy)phenyl]propionic acid;
2-[(E)-2-(3,4-dimethoxyphenyl)ethenyl]-6-methoxyquinoline-4-carboxylic acid;
2-(4-Fluoro-3-methoxyphenyl)-6-methoxyquinoline-4-carboxylic acid;
2-(3-iodo-4-methoxyphenyl)-6-methoxyquinoline-4-carboxylic acid;
2-(3-Hydroxyphenyl)-6-methoxyquinoline-4-carboxylic acid;
2-(4-Hydroxy-3,5-dimethoxyphenyl)-6-methoxyquinoline-4-carboxylic acid;
2-(3,5-Difluoro-4-methoxyphenyl)-6-methoxyquinoline-4-carboxylic acid;
2-(3-Ethylphenyl)-6-methoxyquinoline-4-carboxylic acid;
2-(3-Fluoro-4-methoxyphenyl)-6-methoxyquinoline-4-carboxylic acid;
2-(3-Fluoro-4-methoxyphenyl)-6-methylquinoline-4-carboxylic acid;
6-Methyl-2-(3,4,5-trimethoxyphenyl)quinoline-4-carboxylic acid;
6-Bromo-2-(2,4-dimethylthiazol-5-yl)quinoline-4-carboxylic acid;
2-(7-Methoxybenzofuran-2-yl)-6-trifluoromethoxyquinoline-4-carboxylic acid;
2-(3-Fluoro-4-methoxyphenyl)-6-iodoquinoline-4-carboxylic acid;
2-(3-Fluoro-4-methoxyphenyl)-6-trifluoromethoxyquinoline-4-carboxylic acid;
2-(3-Fluoro-4-methoxyphenyl)-6,8-dimethylquinoline-4-carboxylic acid;
2-(3,4-Dimethoxyphenyl)-6-methoxy-3-methylquinoline-4-carboxylic acid;
2-(4,6-Dimethoxybenzofuran-2-yl)-6-methoxyquinoline-4-carboxylic acid;
6-Methoxy-2-(5-methoxybenzofuran-2-yl)quinoline-4-carboxylic acid;
2-(7-Ethoxybenzofuran-2-yl)-6-methoxyquinoline-4-carboxylic acid;
6-Methoxy-2-(6-methoxybenzofuran-2-yl)quinoline-4-carboxylic acid;
2-(7-Fluorobenzofuran-2-yl)-6-methoxyquinoline-4-carboxylic acid;
2-(4-Fluorobenzofuran-2-yl)-6-methoxyquinoline-4-carboxylic acid;
6-Methoxy-2-(5-methylbenzofuran-2-yl)quinoline-4-carboxylic acid;
6-Methoxy-2-(7-methylbenzofuran-2-yl)quinoline-4-carboxylic acid;
6-Methoxy-2-(4-methoxybenzofuran-2-yl)quinoline-4-carboxylic acid;
6-Methoxy-2-[5-(trifluoromethoxy)benzofuran-2-yl]quinoline-4-carboxylic acid;
4-Ethoxy-3′-fluoro-4′-methoxy[1,1′-biphenyl]-3-carboxylic acid;
4-Ethoxy-3′-methoxy[1,1′-biphenyl]-3-carboxylic acid;
4-Ethoxy-3′-[(methylamino)carbonyl][1,1′-biphenyl]-3-carboxylic acid;
4-Ethoxy-3′,4′,5′-trimethoxy[1,1′-biphenyl]-3-carboxylic acid;
4-Ethoxy-3′,4′-dimethoxy[1,1′-biphenyl]-3-carboxylic acid;
4-Ethoxy-3′-(1-methylethyl)[1,1′-biphenyl]-3-carboxylic acid;
3′,4′,5′-Trimethoxy-4-propoxy[1,1′-biphenyl]-3-carboxylic acid;
3′,4′-Dimethoxy-4-propoxy[1,1′-biphenyl]-3-carboxylic acid;
3′-Methoxy-4-propoxy[1,1′-biphenyl]-3-carboxylic acid;
3′-[(Methylamino)carbonyl]-4-propoxy[1,1′-biphenyl]-3-carboxylic acid;
4,3′,4′,5′-Tetramethoxybiphenyl-3-carboxylic acid;
4,3′,4′-Trimethoxybiphenyl-3-carboxylic acid;
3′-Fluoro-4,4′-dimethoxybiphenyl-3-carboxylic acid;
4,3′-Dimethoxybiphenyl-3-carboxylic acid;
5-Benzo[1,3]dioxol-5-yl-2-methoxybenzoic acid;
3′,4′-Difluoro-4,5′-dimethoxybiphenyl-3-carboxylic acid;
4-Isopropoxy-3′-methoxybiphenyl-3-carboxylic acid;
5-Benzo[1,3]dioxol-5-yl-2-isopropoxybenzoic acid;
4-Isopropoxy-3′,4′,5′-trimethoxybiphenyl-3-carboxylic acid;
3′-Fluoro-4-isopropoxy-4′-methoxybiphenyl-3-carboxylic acid;
4-isopropoxy-3′,4′-dimethoxybiphenyl-3-carboxylic acid;
4-Isopropoxy-3′-methylbiphenyl-3-carboxylic acid;
4′-Fluoro-4-isopropoxy-3′-methylbiphenyl-3-carboxylic acid;
3′,4′-Difluoro-4-isopropoxy-5′-methoxybiphenyl-3-carboxylic acid;
4,3′,4′,5′-Tetramethoxy-5-methylbiphenyl-3-carboxylic acid;
4,3′,4′-Trimethoxy-5-methylbiphenyl-3-carboxylic acid;
3′-Fluoro-4,4′-dimethoxy-5-methylbiphenyl-3-carboxylic acid;
5-Benzo[1,3]dioxol-5-yl-2-methoxy-3-methyl-benzoic acid;
4,3′-Dimethoxy-5-methylbiphenyl-3-carboxylic acid;
4-Methoxy-5,3′-dimethylbiphenyl-3-carboxylic acid;
4′-Fluoro-4-methoxy-5,3′-dimethylbiphenyl-3-carboxylic acid;
3′,4′-Difluoro-4,5′-dimethoxy-5-methylbiphenyl-3-carboxylic acid;
3′-Hydroxy-4-isopropoxybiphenyl-3-carboxylic acid;
3′,4′,5′-Trimethoxy-4-(3-methyl-but-2-enyloxy)biphenyl-3-carboxylic acid;
5-(7-Methoxybenzofuran-2-yl)-2-propoxybenzoic acid;
6-Methoxy-2-(3,4,5-trimethoxyphenyl)quinoline-4-carboxylic acid;
2-(3,4,5-Trimethoxyphenyl)thiazol-4-carboxylic acid;
5-(3,4,5-Trimethoxyphenyl)thiophene-2-carboxylic acid;
5-(3,4,5-Trimethoxyphenyl)-benzo[b]thiophene-2-carboxylic acid;
2-(3-Fluoro-4-methoxyphenyl)-6-methylisonicotinic acid;
2-(3-Fluoro-4-methoxyphenyl)-6-methylpyrimidine-4-carboxylic acid;
6-(4-Methoxyphenyl)-pyrimidine-4-carboxylic acid;
2-(3-Fluoro-4-methoxyphenyl)-6-methoxyquinazoline-4-carboxylic acid;
2-(3-Fluoro-4-methoxyphenyl)-6-iodoquinazoline-4-carboxylic acid;
2-(4-methoxyphenyl)-quinazoline-4-carboxylic acid;
4-Hydroxy-3′,4′,5′-trimethoxybiphenyl-3-carboxylic acid;
4-(3-Cyano-propoxy)-3′,4′,5′-trimethoxybiphenyl-3-carboxylic acid;
4-Cyclopentyloxy-3′-fluoro-4′-methoxybiphenyl-3-carboxylic acid;
4-Cyclopentyloxy-3′-methylbiphenyl-3-carboxylic acid;
3′-(1-Butyl-3-methylureido)-4-cyclopentyloxybiphenyl-3-carboxylic acid;
4-Cyclopentyloxy-4′-fluoro-3′-methylbiphenyl-3-carboxylic acid;
4-Cyclopentyloxy-3′-methoxybiphenyl-3-carboxylic acid;
4-Cyclopentyloxy-3′,4′-dimethoxybiphenyl-3-carboxylic acid;
5-Benzo[1,3]dioxol-5-yl-2-cyclopentyloxybenzoic acid;
4-Cyclopentyloxy-3′,4′,5′-trimethoxybiphenyl-3-carboxylic acid;
4-Cyclopentyloxy-3′,4′-difluoro-5′-methoxybiphenyl-3-carboxylic acid;
3′-[Butyl[(1,1-dimethylethoxy)carbonyl]amino]-4-propoxy[1,1′-bipheny]-3-carboxylic acid;
3′-(1-Butyl-3-methylureido)-4-methoxybiphenyl-3-carboxylic acid;
3′-(1-Butyl-3-methylureido)-4-methoxy-5-methylbiphenyl-3-carboxylic acid;
3′-(1-Butyl-3-methylureido)-4-isopropoxybiphenyl-3-carboxylic acid
N-[(R)-1-(Methoxycarbonyl)-2-(1-ethyl)-1H-indol-3-yl)ethyl]-6-methoxy-2-(3-methoxyphenyl)quinoline-4-carboxamide;
N-[(R)-1-(Methoxycarbonyl)-2-(1-propyl-1H-indol-3-yl)ethyl]-6-methoxy-2-(3,4,5-trimethoxyphenyl)quinoline-4-carboxamide;
N-[(R)-1-(Methoxycarbonyl)-2-(1-ethyl)-1H-indol-3-yl)ethyl]-2-(3,5-difluoro-4-methoxyphenyl)-6-methoxyquinoline-4-carboxamide;
N-[(R)-1-(Methoxycarbonyl)-2-(1-isopropyl-1H-indol-3-yl)ethyl]-6-methoxy-2 (3-methoxyphenyl)-quinoline-4-carboxamide;
N-[(R)-1-(Methoxycarbonyl)-2-(1-isopropyl-1H-indol-3-yl)ethyl]-2-(3,5-difluoro-4-methoxyphenyl)-6-methoxyquinoline-4-carboxamide;
N-[(R)-1-(Methoxycarbonyl)-2-(1-isopropyl-1H-indol-3-yl)ethyl]-6-methoxy-2-(3,4,5-trimethoxyphenyl)quinoline-4-carboxamide;
N-[(R)-1-(Methoxycarbonyl)-2-(1-ethyl)-1H-indol-3-yl)ethyl]-2-(3-fluoro-4-methoxyphenyl)-6-trifluoromethoxyquinoline-4-carboxamide;
N-[(R)-1-(Methoxycarbonyl)-2-(1-ethyl)-1H-indol-3-yl)ethyl]-2-(7-methoxybenzofuran-2-yl)-6-trifluoromethoxyquinoline-4-carboxamide;
N-[(R)-1-(Methoxycarbonyl)-2-(1-isopropyl-1H-indol-3-yl)ethyl]-2-(3-fluoro-4-methoxyphenyl)-6-trifluoromethoxyquinoline-4-carboxamide;
N-[(R)-1-(Methoxycarbonyl)-2-(1-isopropyl-1H-indol-3-yl)ethyl]-2-(7-methoxybenzofuran-2-yl)-6-trifluoromethoxyquinoline-4-carboxamide;
N-[(R)-1-(Methoxycarbonyl)-2-(1-n-hexyl-1H-indol-3-yl)ethyl]-6-methoxy-2-(3,4,5-trimethoxyphenyl)quinoline-4-carboxamide;
N-[(R)-1-(Methoxycarbonyl)-2-(1-ethyl)-1H-indol-3-yl)ethyl]-4-ethoxy-3′-methoxybiphenyl-3-carboxamide;
N-[(R)-1-(Methoxycarbonyl)-2-(1-isopropyl)-1H-indol-3-yl)ethyl]-4-ethoxy-3′-methoxybiphenyl)-3-carboxamide;
6-Bromoquinoline-8-carboxylic acid [(R)-1-hydroxymethyl-2-(1H-indol-3-yl)ethyl]amide;
3-Bromonaphthalene-1-carboxylic acid [(R)-1-hydroxymethyl-2-(1H-indol-3-yl)ethyl]amide;
5-Bromo-4-methoxythiophene-3-carboxylic acid [(R)-2-hydroxy-1-(1H-indol-3-ylmethyl)ethyl]amide;
6-Bromo-1H-benzimidazole-4-carboxylic acid [(R)-2-hydroxy-1-(1H-indol-3-ylmethyl)ethyl]amide;
5-Bromo-2-ethoxy-N-[(R)-2-hydroxy-1-(1H-indol-3-ylmethyl)ethyl]benzamide;
N-[(R)-1-Hydroxymethyl-2-(1H-indol-3-yl)ethyl]-5-iodo-2-propoxybenzamide;
N-[(R)-1-Hydroxymethyl-2-(1-methyl-1H-indol-3-yl)ethyl]-5-iodo-2-propoxybenzamide;
N-[2-(5-Fluoro-1H-indol-3-yl)-1-hydroxymethylethyl]-5-iodo-2-propoxybenzamide
   and the methyl, ethyl, propyl and butyl esters thereof.
   Pharmacological Investigations
   HTRF Assay for Measuring cAMP in Cells

The method is based on a competitive immunoassay between native cAMP, which has been produced by the cells, and cAMP which is labelled with XL665. The tracer binding was visualized by a monoclonal antibody, anti-cAMP labelled with cryptate [HTRF=homogeneous time-resolved fluorescence].

The specific signal is inversely proportional to the cAMP concentration of the samples employed.

The 665 nm/620 nm fluorescence ratio was evaluated.

The following material was used: 96-well plates for the tissue culture, 96-well plates with black edge and black base (e.g. Fluotrac 600 from Greiner), 96-well plates for the substance dilutions of polypropylene and cAMP Femtomolar (4000 wells Kit, CIS Bio International # 62AM1PEC).

The following reagents were used: BSA (bovine serum albumin) Fraction V protease-free, IBMX (3-isobutyl-1-methylxanthine), hFSH (human follicle stimulating hormone), Triton X-100 analytical grade, potassium fluoride analytical grade, G 418 (Geneticin) and Accutase.

Buffer 1 (washing and testing buffer) contained PBS, 1 mM CaCl2, 1 mM MgCl₂, 0.2% glucose; 0.1% BSA, 1 mM IBMX.

Buffer 2 (2× lysis buffer) contained 1% Triton X-100 in PBS (without CaCl₂ and MgCl₂).

Buffer 3 (assay buffer) contained 50 mM potassium phosphate buffer (pH 7.0); 800 mM potassium fluoride; 0.2% BSA (always added fresh).

Procedure:

On day 1, the cells were seeded in 96-well plates (3×10⁴ cells per well hFSHR clone 16 cells (CHO cells stably transfected with the human FSH receptor in 150 μl of medium).

The next day, test substance dilutions were made up. For this purpose, all the substances were diluted in ice-cold buffer 1 (with or without hFSH), and the substance dilutions were placed on ice until applied to the cells.

The cell supernatant was then aspirated off, and the cells were washed 2× with 200 μl of buffer 1. The cells were treated with 60 μl of the appropriate substance concentrations at 37° C. for 2 h. The cells were then lysed with 60 μl of buffer 2 (put onto the supernatant) (on a plate shaker at RT for 30 min).

The test conjugates (XL-665 and anti-cAMP cryptate) were diluted in buffer 3 in accordance with the manufacturers' information. The actual mixture for measurement was pipetted into a black 96-well plate (in each case 15 μl of the cell lysate diluted with 35 μl of buffer 1; firstly 25 μl of XL-665 conjugate were pipetted and, after 10 min, 25 μl of the anti-cAMP cryptate were added). This is followed by incubation at RT for 90 minutes. The measurement was carried out in a PheraStar (BMG).

Tissue culture conditions

| | |
|---|---|
| 1) hFSHr clone 16 | Ham's F12 |
| | PSG |
| | 10% FCS |
| | 700 μg/ml G 418 (Geneticin) from PAA. |

Dose-effect curve (hFSH) for the human receptor: 1e-8, 3e-9, 1e-9, 3e-10, 1e-10, 3e-11, 1e-11, 3e-12 mol/l.

The test substances were employed in suitable dilutions in the absence (test for agonism) and in the presence of 1e-9 mol/l hFSH.

Evaluation

The values of the well ratio were averaged and then entered directly in SigmaPlot versus the concentrations. The maximum and minimum values were determined for each plate, and half the difference is to be regarded as IC₅₀.

The test results (Table 1) show that the compounds according to the invention have an FSH-antagonistic effect.

TABLE 1
FSH-antagonistic effect of selected compounds in the HTRF assay

| Compound [Ex. #] | IC₅₀ | |
|---|---|---|
| 1 | 7 | μM |
| 8 | 1 | μM |
| 9 | 200 | nM |
| 10 | 1 | μM |
| 16 | 4 | μM |
| 17 | 400 | nM |
| 19 | 6 | μM |
| 22 | 300 | nM |
| 26 | 9 | μM |
| 36 | 6 | μM |
| 38 | 900 | nM |
| 59 | 6 | μM |
| 86 | 8 | μM |
| 87 | 10 | μM |
| 88 | 10 | μM |
| 91 | 6 | μM |
| 96 | 7 | μM |
| 97 | 4 | μM |
| 108 | 3 | μM |
| 120 | 1.5 | μM |
| 130 | 4 | μM |

TABLE 2
(continuation) FSH-antagonistic effect of selected compounds
in the HTRF assay

| Compound [Ex. #] | IC₅₀ | |
|---|---|---|
| 162 | 1.5 | μM |
| 307 | 450 | nM |
| 333 | 450 | nM |
| 337 | 3.5 | μM |
| 345 | 1 | μM |
| 361 | 4 | μM |
| 368 | 2.5 | μM |
| 373 | 8 | μM |
| 379 | 4.5 | μM |
| 388 | 400 | nM |
| 392 | 1.5 | μM |
| 396 | 3.5 | μM |
| 403 | 100 | nM |
| 418 | 300 | nM |
| 430 | 400 | nM |
| 483 | 1 | μM |

Dosage

Satisfactory results are generally to be expected if the daily doses comprise a range from 5 μg to 50 mg of the compound according to the invention per kg of body weight. A recommended daily dose for larger mammals, for example humans, is in the range from 10 μg to 30 mg per kg of body weight. Suitable dosages for the compounds according to the invention are from 0.005 to 50 mg per day per kg of body weight, depending on the age and constitution of the patient, it being possible to administer the necessary daily dose by single or multiple delivery.

Pharmaceutical products based on the novel compounds are formulated in a manner known per se by processing the active ingredient with the carrier substances, fillers, substances which influence disintegration, binders, humectants, lubricants, absorbents, diluents, test modifiers, colorants etc. which are used in pharmaceutical technology, and converting into the desired administration form. Reference should be made in this connection to Remington's Pharmaceutical Science, 15^{th} ed. Mack Publishing Company, East Pennsylvania (1980).

Suitable for oral administration are in particular tablets, coated tablets, capsules, pills, powders, granules, pastilles, suspensions, emulsions or solutions. Preparations for injection and infusion are possible for parenteral administration. Appropriately prepared crystal suspensions can be used for intraarticular injection. Aqueous and oily solutions for injection or suspensions and corresponding depot preparations can be used for intramuscular injection. The novel compounds can be used for rectal administration in the form of suppositories, capsules, solutions (e.g. in the form of enemas) and ointments both for systemic and for local therapy. Formulations possible for topical application are gels, ointments, greasy ointments, creams, pastes, dusting powders, milk and tinctures. The dosage of the compounds of the general formula I in these preparations should be 0.01%-20% in order to achieve an adequate pharmacological effect. Topical use can also take place by means of a transdermal system, for example a patch.

The invention likewise encompasses the compounds according to the invention of the general formula I as therapeutic active ingredient. The invention further includes the compounds according to the invention of the general formula I as therapeutic active ingredients together with pharmaceutically suitable and acceptable excipients and carriers. The invention likewise encompasses a pharmaceutical composition which comprises one of the pharmaceutically active compounds according to the invention or mixture thereof and a pharmaceutically suitable salt or pharmaceutically suitable excipients and carriers.

The present invention therefore also relates to pharmaceutical compositions which comprise at least one compound of the general formula I, where appropriate together with pharmaceutically suitable excipients and/or carriers.

Suitable for forming pharmaceutically suitable salts of the compounds according to the invention of the general formula I are, by methods known to the skilled person, as inorganic acids inter alia hydrochloric acid, hydrobromic acid, sulphuric acid and phosphoric acid, nitric acid, as carboxylic acids inter alia acetic acid, propionic acid, hexanoic acid, octanoic acid, decanoic acid, oleic acid, stearic acid, maleic acid, fumaric acid, succinic acid, benzoic acid, ascorbic acid, oxalic acid, salicylic acid, tartaric acid, citric acid, lactic acid, glycolic acid, malic acid, mandelic acid, cinnamic acid, glutamic acid, aspartic acid, and as sulphonic acids inter alia methanesulphonic acid, ethanesulphonic acid, toluenesulphonic acid, benzenesulphonic acid and naphthalenesulphonic acid.

These pharmaceutical compositions and medicaments may be intended for oral, rectal, subcutaneous, transdermal, percutaneous, intravenous or intramuscular administration.

They comprise besides conventional carriers and/or diluents at least one compound of the general formula I.

The medicaments of the invention are produced using the customary solid or liquid carriers or diluents and the excipients customarily used in pharmaceutical technology, in accordance with the desired mode of administration with a suitable dosage in a known manner. The preferred preparations consist of a dosage form which is suitable for oral administration.

Examples of such dosage forms are tablets, film-coated tablets, sugar-coated tablets, capsules, pills, powders, solutions or suspensions or else depot forms.

The pharmaceutical compositions which comprise at least one of the compounds according to the invention are preferably administered orally.

Parenteral preparations such as solutions for injection are also suitable. Preparations which may also be mentioned for example are suppositories.

Appropriate tablets can be obtained for example by mixing the active ingredient with known excipients, for example inert diluents such as dextrose, sugar, sorbitol, mannitol, polyvinylpyrrolidone, disintegrants such as maize starch or alginic acid, binders such as starch or gelatine, lubricants such as magnesium stearate or talc and/or agents to achieve a depot effect such as carboxylpolymethylene, carboxylmethylcellulose, cellulose acetate phthalate or polyvinyl acetate. The tablets may also consist of a plurality of layers.

Correspondingly, coated tablets can be produced by coating cores which have been produced in analogy to the tablets with agents normally used in tablet coatings, for example polyvinylpyrrolidone or shellac, gum Arabic, talc, titanium oxide or sugar. The tablet coating may also consist of a plurality of layers, it being possible to use the excipients mentioned above for tablets.

Solutions or suspensions with the compounds according to the invention of the general formula I may additionally comprise taste-improving agents such as saccharin, cyclamate or sugar and, for example, flavourings such as vanillin or orange extract. They may additionally comprise suspending aids such as sodium carboxymethylcellulose or preservatives such as p-hydroxybenzoates.

Capsules comprising the compounds of the general formula I can be produced for example by the compound(s) of the general formula I being mixed with an inert carrier such as lactose or sorbitol and encapsulated in gelatine capsules.

Suitable suppositories can be produced for example by mixing with carriers intended for this purpose, such as neutral fats or polyethylene glycol or derivatives thereof.

The compounds according to the invention of the general formula I can be prepared as described below.

Abbreviations Used:

ACN Acetonitrile
DIBAC Diisobutylaluminium hydride
DMF N,N-Dimethylformamide
EDC N-Ethyl-N′-(3-dimethylaminopropyl)carbodiimide
EtOH Ethanol
HATU O-(7-Azabenzotriazol-1-yl)-N,N,N′,N′-tetramethyluronium hexafluorophosphate
FMOC (9H-Fluoren-9-ylmethoxy)carbonyl
HOBt 1-Hydroxy-1H-benzotriazole
MeCN Acetonitrile
MeOH Methanol
MTBE Methyl tert-butyl ether
NMM 4-methylmorpholine
NMP N-Methylpyrrolidinone
Rf Reflux
RT Room temperature
TBAF Tetrabutylammonium fluoride
TFA Trifluoroacetic acid
THF Tetrahydrofuran

Compounds of the general formula I or Ia can in principle be prepared as shown in Scheme 4 by an amide-formation reaction between a tryptophanol derivative VI or VIa and a carboxylic acid VII. The reagents typically used for the coupling are EDC and HOBt.

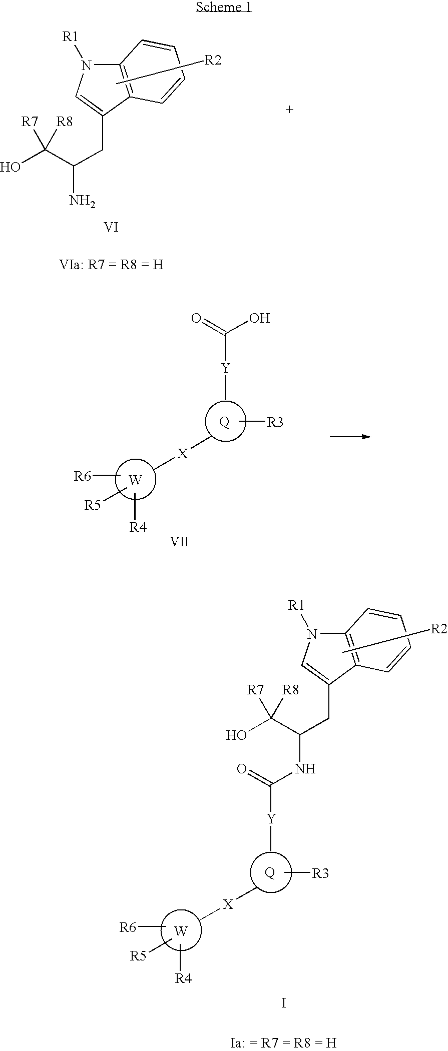

The tryptophanol derivatives of the formula VI can be prepared as shown in Scheme 5 from the corresponding amino acids which can be purchased or are known from the literature.

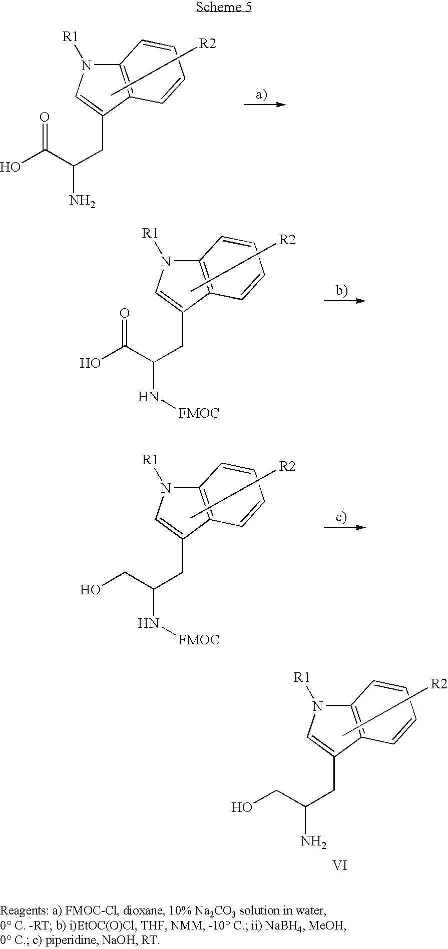

The carboxylic acids of the general formula VII can be prepared as shown in Scheme 6 by a Suzuki reaction between a boronic acid XII or XVI and a halogen compound XIII or XV (Hal ═I, Br, Cl).

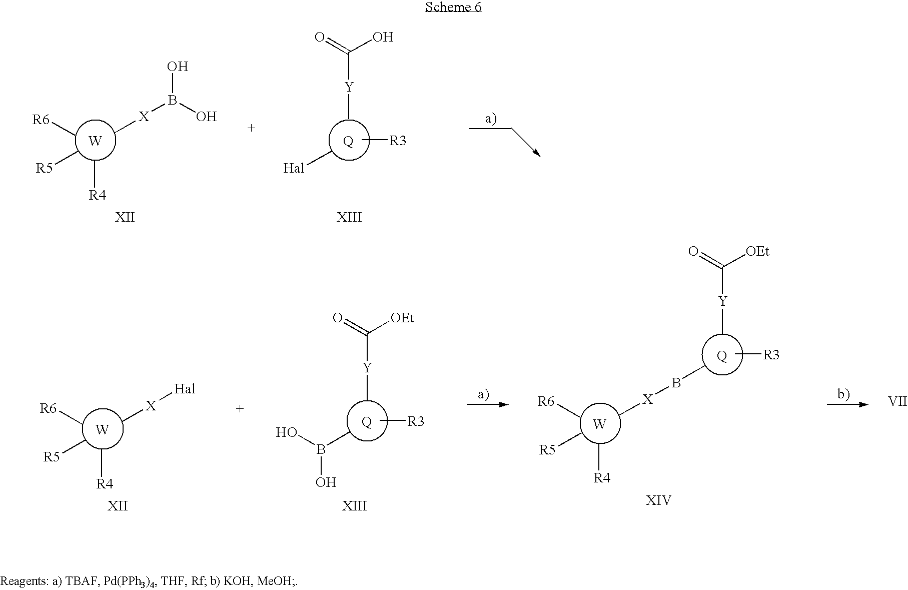

Carboxylic acids of the formula XIX can be prepared as shown in Scheme 7 in a so-called Pfitzinger reaction from a methyl ketone and an isatin derivative XVIII.

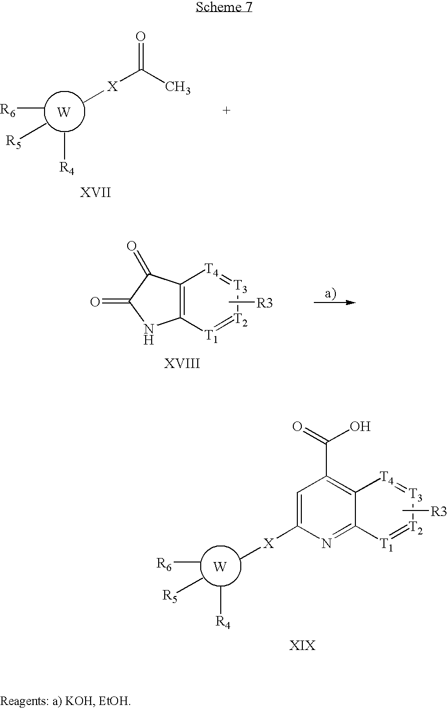

Carboxylic acids of the general formulae XXI and XXII can likewise be prepared by a Pfitzinger reaction as shown in Scheme 8.

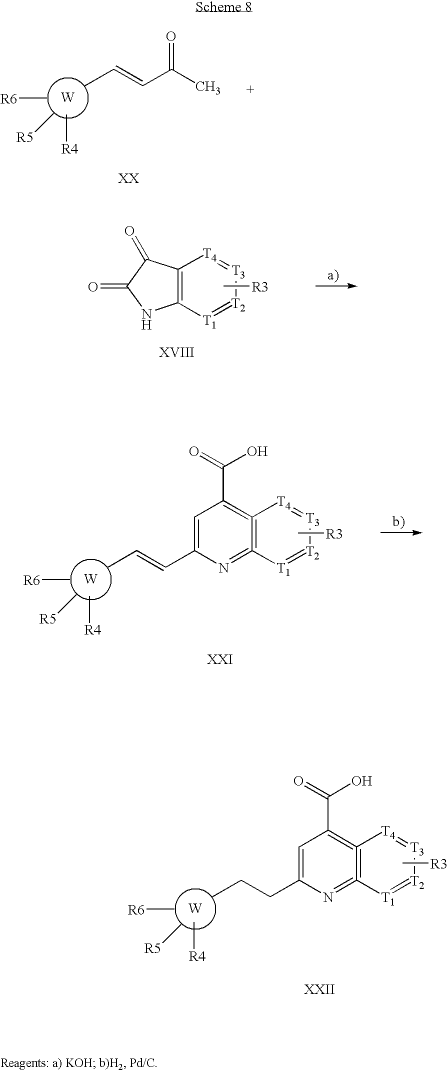

Carboxylic acids of the general formula XXVIII can be prepared in an ether synthesis as shown in Scheme 9.

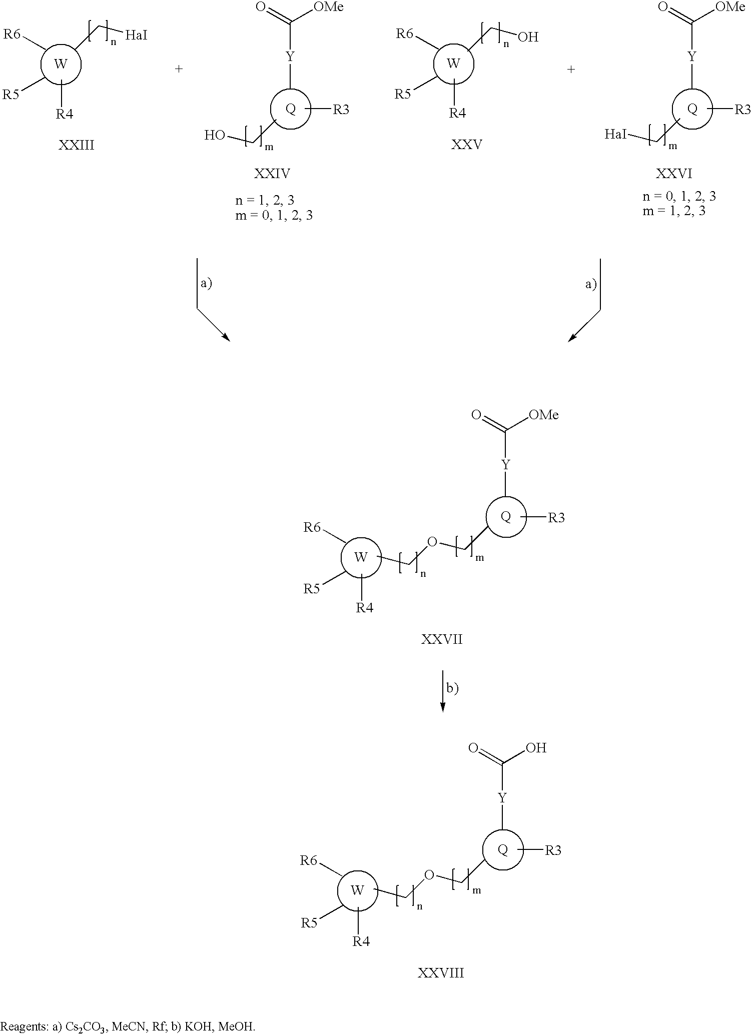

### Synthesis of the Compounds According to the Invention

### EXAMPLE 1

### N-[(R,S)-2-(5-Bromo-1H-indol-3-yl)-1-(hydroxymethyl)ethyl]-2-(3,4,5-trimethoxyphenyl)quinoline-4-carboxamide

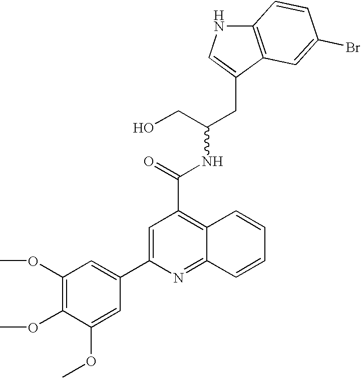

### 1a) (R,S)-5-Bromo-α-[[(9H-fluoren-9-ylmethoxy)carbonyl]amino]-1H-indole-3-propanoic acid

A solution of 0.36 mmol (92 mg) of 9-fluorenylmethyl chloroformate in 1.11 ml of dioxane was slowly added, while stirring and cooling to 0° C. in an ice bath, to a solution of 0.35 mmol (100 mg) of 5-bromo-DL-tryptophan in 0.55 ml of dioxane and 1.11 ml of 10% strength aqueous sodium carbonate solution. After the addition was complete, the mixture was stirred at 0° C. for one hour and at room temperature for a further three hours, cooled again to 0° C. and 24 ml of water were added dropwise. Then 1.0 ml of concentrated hydrochloric acid is used to acidify, whereupon the protected amino acid precipitated. After the precipitate had been stored in a refrigerator and filtered, 163 mg of white amorphous solid product were obtained.

¹H-NMR (400 MHz, DMSO-d₆): δ [ppm]=12.73 s (1H, COOH); 11.07 s (1H, NH); 7.87 d (J=7.5 Hz, 2H, aryl); 7.75 s (1H, aryl); 7.70 d (J=8.1 Hz, 1H, aryl); 7.63 m (2H, aryl); 7.39 m (2H, aryl); 7.27 m (4H, aryl); 7.17 d (J=6.9 Hz, 1H, aryl); 4.18 m (2H, CH); 3.56 s (2H, OCH₂); 3.18 dd (J=14.5 Hz/4.7 Hz, 1H, CH); 3.14 dd (J=14.5 Hz/4.4 Hz, 1H, CH).

MS (ESI, +): 505 (M+1).

### 1b,c) (9H-Fluoren-9-ylmethyl)[(R,S)-2-(5-bromo-1H-indol-3-yl)-1-(hydroxymethyl)ethyl]carbamate

0.32 mmol (35 μl) of N-methylmorpholine was added to a stirred solution of 0.32 mmol (163 mg) of the protected amino acid prepared as in 1a) in 1.7 ml of THF at −10° C., followed by 0.32 mmol (31 μl) of ethyl chloroformate. The mixture was then stirred for a further hour at the stated temperature. Subsequently, 0.96 mmol (36 mg) of sodium borohydride was added in one portion.

When the reaction mixture had reached the temperature of 0° C., 3.2 ml of methanol were added dropwise. The solution was stirred for a further 10 minutes and then neutralized with 0.4 ml of 1 M hydrochloric acid. The organic solvents were removed in vacuo. The residue was taken up in water and extracted with methyl tertiary butyl ether. The resulting organic phase was dried over magnesium sulphate, filtered and concentrated in vacuo. 157 mg of the target compound were obtained as a colourless foam.

¹H-NMR (400 MHz, DMSO-d₆): δ [ppm]=11.00 s (1H, NH); 7.86 d (J=7.5 Hz, 2H, aryl); 7.76 s (1H, aryl); 7.64 m (2H, aryl); 7.39 m (2H, aryl); 7.29 m (3H, aryl); 7.16 m (3H, aryl); 4.74 t (J=5.6 Hz, 1H, OH); 4.17 m (4H, CH, OCH₂); 3.74 m (2H, OCH₂); 2.91 dd (J=14.3 Hz/5.8 Hz, 1H, CH); 2.70 dd (J=14.4 Hz/8.4 Hz, 1H, CH).

MS (ESI,+): 491 (M+1).

### 1d) (R,S)-β-Amino-5-bromo-1H-indole-3-propanol

0.30 mmol (150 mg) of the protected amino alcohol prepared as in 1 b,c) was stirred in 4 ml of piperidine at room temperature for one hour. After the solution had been cooled to 0° C., 2 ml of water were added dropwise. The resulting precipitate was filtered off, and a total of 1.5 g of potassium hydroxide powder was added in portions to the filtrate while stirring. The piperidine phase was separated off and concentrated in vacuo with addition of toluene. 110 mg of the amino alcohol still contaminated with piperidine were obtained.

MS (ESI,+): 269(M+1).

### 1e) N-[(R,S)-2-(5-Bromo-1H-indol-3-yl)-1-(hydroxymethyl)ethyl]-2-(3,4,5-trimethoxyphenyl)quinoline-4-carboxamide

0.38 mmol (130 mg) of 2-(3,4,5-trimethoxyphenyl)quinoline-4-carboxylic acid were dissolved in 3 ml of DMF and, at room temperature, 0.38 mmol (59 mg) of 1-hydroxy-1H-benzotriazole hydrate and 0.38 mmol (73 mg) of N-[3-(dimethylamino)propyl]-N′-ethylcarbodiimide hydrochloride were added. The mixture was stirred at the stated temperature for 30 minutes and then about 0.35 mmol (100 mg) of the amino alcohol obtained as in 1d) was added.

After a further hour, the reaction mixture was added to saturated aqueous sodium hydrogen carbonate solution, and the precipitate was filtered and washed with water. Purification by chromatography on silica gel with the eluent cyclohexane/ethyl acetate affords 50 mg of the amide as yellowish solid.

¹H-NMR (400 MHz, DMSO-d₆): δ [ppm]=11.09 s (1H, NH); 8.64 d (J=8.3 Hz, 1H, aryl); 8.07 d (J=8.4 Hz, 1H, aryl); 7.98 s (1H, aryl); 7.82 s (1H, aryl); 7.74 m (2H, aryl); 7.53 s (2H, aryl); 7.46 t (J=7.5 Hz, 1H, aryl); 7.34 d (J=8.5 Hz, 1H, aryl); 7.26 s (1H, aryl); 7.16 d (J=7.3 Hz, 1H, aryl); 4.92 t (J=5.0 Hz, 1H, OH); 4.36 m (1H, CH); 3.93 s (6H, OCH₃); 3.76 s (3H, OCH₃); 3.59 m (2H, OCH₂); 3.06 dd (J=14.6 Hz/5.4 Hz, 1H, CH); 2.89 dd (J=14.6 Hz/8.5 Hz, 1H, CH).

MS (APCl, −): 588 (M−1).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 2 | N-[(R,S)-1-(Hydroxymethyl)-2- (5-methyl-1H-indol-3-yl)ethyl]-2- (3,4,5-trimethoxy- phenyl)quinoline-4- carboxamide; (R,S)-β-Amino-5-methyl-1H- indole-3-propanol and 2-(3,4,5- Trimethoxyphenyl)quinoline-4- carboxylic acid | 1 | (DMSO-d₆): 10.72 s (1H, NH); 8.63 d (J=8.3 Hz, 1H, NH); 8.07 d (J=8.4 Hz, 1H, aryl); 7.97 s (1H, aryl); 7.81 d (J=8.4 Hz, 1H, aryl); 7.76 t (J=7.6 Hz, 1H, aryl); | |
| # 7.52 s (2H, aryl); 7.49 t (J=7.6 Hz, 1H, aryl); 7.41 s (1H, aryl); 7.24 d (J=8.2 Hz, 1H, aryl); 7.14 s (1H, aryl); 6.88 d (J=8.2 Hz, 1H, aryl); 4.89 t (J=5.7 Hz, 1H, OH); 4.38 m (1H, CH); 3.93 s (6H, OCH₃); 3.76 s (3H, OCH₃); 3.60 m (2H, OCH₂); 3.06 dd (J=14.4 Hz/5.5 Hz, 1H, CH); 2.89 dd (J=14.4 Hz/8.3 Hz, 1H, CH); 2.30 s (3H, CH₃). MS (ESI; +): 526. | 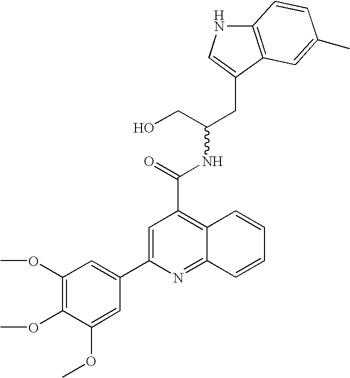 | | | |
| 3 | N-[(R,S)-1-(Hydroxymethyl)-2- (4-methyl-1H-indol-3-yl)ethyl]-2- (3,4,5-trimethoxyphenyl)- quinoline-4-carboxamide; (R,S)-β-Amino-4-methyl-1H- indole-3-propanol and 2-(3,4,5- Trimethoxyphenyl)quinoline-4- carboxylic acid | 1 | (DMSO-d₆): 10.82 s (1H, NH); 8.66 d (J=8.6 Hz, 1H, NH); 8.07 d (J=8.4 Hz, 1H, aryl); 8.01 s (1H, aryl); 7.78 m (2H, aryl); 7.54 s (2H, aryl); 7.50 t (J=7.6 Hz, 1H, aryl); | |
| # 4.97 t (J=5.7 Hz, 1H, OH); 4.39 m (1H, CH); 3.93 s (6H, OCH₃); 3.76 s (3H, OCH₃); 3.67 m (1H, OCH); 3.59 m (1H, OCH); 3.31 m (1H, CH); 3.00 m (1H, CH); 2.70 s (3H, CH₃). MS (ESI; +): 526 (M + 1). | 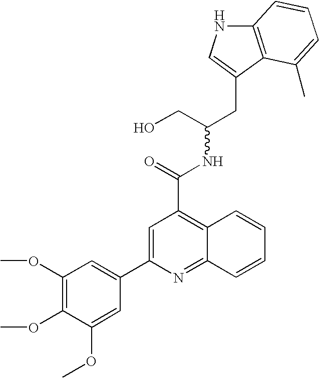 | | | |
| 4 | N-[(R,S)-1-(Hydroxymethyl)-2- (6-methyl-1H-indol-3-yl)ethyl]-2- (3,4,5-trimethoxyphenyl)- quinoline-4-carboxamide; (R,S)-β-Amino-6-methyl-1H- indole-3-propanol and 2-(3,4,5- Trimethoxyphenyl)quinoline-4- carboxylic acid | 1 | (DMSO-d₆): 10.68 s (1H, NH); 8.62 d (J=8.4 Hz, 1H, NH); 8.07 d (J=8.4 Hz, 1H, aryl); 7.99 s (1H, aryl); 7.82 d (J=8.3 Hz, 1H, aryl); 7.76 t (J=7.6 Hz, 1H, aryl); 7.53 | |
| # s (2H, aryl); 7.49 m (J=7.7 Hz, 2H, aryl); 7.12 s (1H, aryl); 7.10 s (1H, aryl); 6.78 d (J=8.1 Hz, 1H, aryl), 4.88 t (J=5.5 Hz, 1H, OH); 4.38 m (1H, CH); 3.92 s (6H, OCH₃); 3.76 s (3H, OCH₃); 3.59 m (2H, OCH₂); 3.05 dd (J=14.5 Hz/5.7 Hz, 1H, CH); 2.90 dd (J=14.4 Hz/8.3 Hz, 1H, CH); 2.37 s (3H, CH₃). MS (APCI; −): 524 (M − 1). | 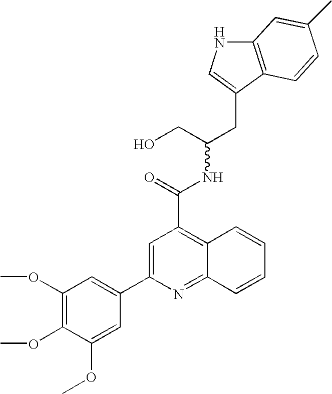 | | | |
| 5 | N-[(R,S)-1-(Hydroxymethyl)-2-(1- methyl-1H-indol-3-yl)ethyl]-2- (3,4,5-trimethoxyphenyl)- quinoline-4-carboxamide (R)-β-Amino-1-methyl-1H- indole-3-propanol 2-(3,4,5- Trimethoxyphenyl)quinoline-4- carboxylic acid | 1 | (DMSO-d₆): 8.65 d (J=8.4 Hz, 1H, NH); 8.08 d (J=8.4 Hz, 1H, aryl); 8.03 s (1H, aryl); 7.81 d (J=8.4 Hz, 1H, aryl); 7.77 t (J=7.6 Hz, 1H, aryl); 7.68 d (J=7.9 Hz, 1H, aryl); 7.55 | |
| # s (2H, aryl); 7.48 t (J=7.5 Hz, 1H, aryl); 7.41 d (J=8.2 Hz, 1H, aryl); 7.16 s (1H, aryl); 7.13 t (J=7.8 Hz, 1H, aryl); 7.00 t (J=7.4 Hz, 1H, aryl); 4.90 t (J=5.7 Hz, 1H, OH); 4.38 m (1H, CH); 3.93 s (6H, OCH₃); 3.76 s (3H, OCH₃); 3.74 s (3H, NCH₃); 3.60 m (2H, OCH₂); 3.07 dd (J=14.5 Hz/5.8 Hz, 1H, CH); 2.91 dd (J=14.5 Hz/8.1 Hz, 1H, CH). MS (ESI; +): 526 (M + 1). | 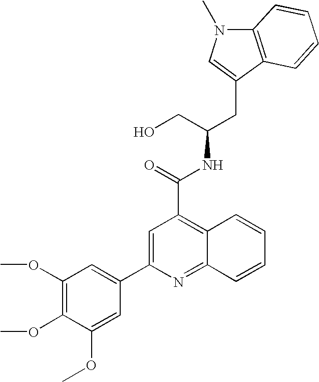 | | | |
| 6 | N-[(R,S)-2-(5-Fluoro-1H-indol-3- yl)-1-(hydroxymethyl)ethyl]-2- (3,4,5-trimethoxyphenyl)- quinoline-4-carboxamide; (R,S)-β-Amino-5-fluoro-1H- indole-3-propanol 2-(3,4,5- Trimethoxyphenyl)quinoline-4- carboxylic acid | 1 | (DMSO-d₆): 10.96 s (1H, NH); 8.65 d (J=8.4 Hz, 1H, NH); 8.07 d (J=8.6 Hz, 1H, aryl); 8.00 s (1H, aryl); 7.76 m (2H, aryl); 7.54 s (2H, aryl); 7.47 t (J=7.5 Hz, 1H, aryl); 7.40 d (J=10.0 | |
| # Hz, 1H, aryl); 7.35 m (1H, aryl); 7.28 s (1H, aryl); 6.89 t (J=10.2 Hz, 1H, aryl); 4.91 t (J=5.4 Hz, 1H, OH); 4.37 m (1H, CH); 3.93 s (6H, OCH₃); 3.76 s (3H, OCH₃); 3.60 m (2H, OCH₂); 3.05 dd (J=14.4 Hz/5.6 Hz, 1H, CH); 2.89 dd (J=14.4 Hz/8.1 Hz, 1H, CH). ¹⁹F-NMR (400 MHz, DMSO-d₆): −124.84 m (1F). MS (APCI; −): 528 (M − 1). | 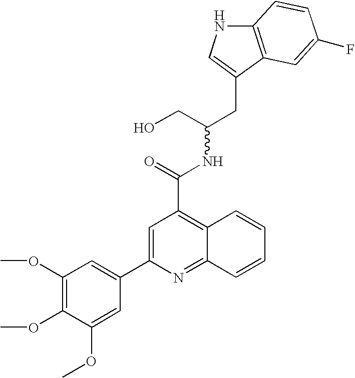 | | | |
| 7 | N-[(R,S)-1-(Hydroxymethyl)-2- (5-methoxy-1H-indol-3-yl)ethyl]- 2-(3,4,5- trimethoxyphenyl)quinoline-4- carboxamide; (R,S)-β-Amino-5-methoxy-1H- indole-3-propanol and 2-(3,4,5- Trimethoxyphenyl)quinoline-4- carboxylic acid | 1 | (DMSO-d₆): 10.69 s (1H, NH); 8.65 d (J=8.4 Hz, 1H, NH); 8.07 d (J=8.3 Hz, 1H, aryl); 7.99 s (1H, aryl); 7.82 d (J=8.3 Hz, 1H, aryl); 7.76 t (J=7.0 Hz, 1H, aryl); 7.53 s | |
| # (2H, aryl); 7.48 t (J=7.5 Hz, 1H, aryl); 7.24 d (J=8.7 Hz, 1H, aryl); 7.16 s (2H, aryl); 6.71 d (J=8.7 Hz, 1H, aryl); 4.89 t (J=5.7 Hz, 1H, OH); 4.38 m (1H, CH); 3.93 s (6H, OCH₃); 3.76 s (3H, OCH₃); 3.68 s (3H, OCH₃); 3.60 m (2H, OCH₂); 3.04 dd (J=14.5 Hz/5.6 Hz, 1H, CH); 2.90 dd (J=14.5 Hz/8.3 Hz, 1H, CH). MS (ESI; +): 542 (M + 1). | 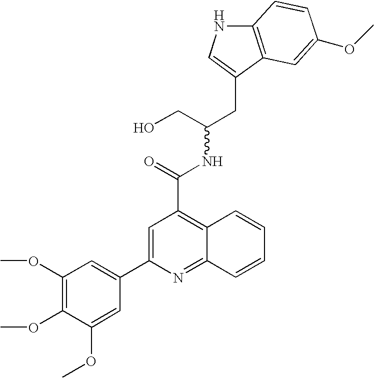 | | | |
| 8 | N-[(R,S)-2-(6-Fluoro-1H-indol-3- yl)-1-(hydroxymethyl)ethyl]-2- (3,4,5-trimethoxyphenyl)- quinoline-4-carboxamide; (R,S)-β-Amino-6-fluoro-1H- indole-3-propanol and 2-(3,4,5- Trimethoxyphenyl)quinoline-4- carboxylic acid | 1 | (DMSO-d₆): 10.93 s (1H, NH); 8.64 d (J=8.3 Hz, 1H, NH); 8.07 d (J=8.4 Hz, 1H, aryl); 8.00 s (1H, aryl); 7.78 m (2H, aryl); 7.64 m (1H, aryl); 7.54 s (2H, aryl); 7.48 t (J=7.5 Hz, | |
| # 1H, aryl); 7.20 s (1H, aryl); 7.19 d (J=10.2 Hz, 1H, aryl); 6.82 t (J=8.0 Hz, 1H, aryl); 4.91 t (J=5.7 Hz, 1H, OH); 4.38 m (1H, CH); 3.93 s (6H, OCH₃); 3.76 s (3H, OCH₃); 3.59 m (2H, OCH₂); 3.06 dd (J=14.5 Hz/5.6 Hz, 1H, CH); 2.91 dd (J=14.5 Hz/8.3 Hz, 1H, CH). ¹⁹F-NMR (400 MHz, DMSO-d₆): −121.73 m (1F). MS (ESI; +): 530 (M + 1). | 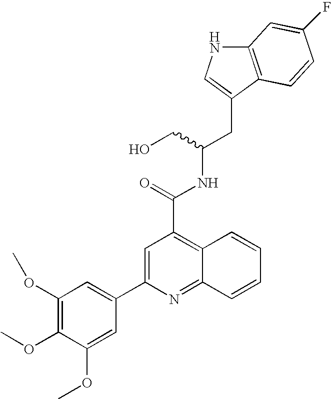 | | | |
| 9 | N-[(R,S)-1-(Hydroxymethyl)-2- [5-(phenylmethoxy)-1H-indol-3- yl]ethyl]-2-(3,4,5- trimethoxyphenyl)quinoline-4- carboxamide; (R,S)-β-Amino-5-methoxy-1H- indole-3-propanol and 2-(3,4,5- Trimethoxyphenyl)quinoline-4- carboxylic acid | 1 | (DMSO-d₆): 10.71 s (1H, NH); 8.66 d (J=8.4 Hz, 1H, NH); 8.07 d (J=8.4 Hz, 1H, aryl); 7.96 s (1H, aryl); 7.81 d (J=8.4 Hz, 1H, aryl); 7.76 t (J=7.5 Hz, 1H, | |
| # aryl); 7.51 s (1H, aryl); 7.48 t (J=7.5 Hz, 1H, aryl); 7.32 m (8H, aryl); 7.18 s (1H, aryl); 6.77 d (J=8.7 Hz, 1H, aryl); 4.98 d (J=11.7 Hz, 1H, OCH); 4.38 m (1H, CH); 3.91 s (6H, OCH₃); 3.75 s (3H, OCH₃); 3.61 m (2H, OCH₂); 3.04 dd (J=14.4 Hz/5.4 Hz, 1H, CH); 2.89 dd (J=14.5 Hz/8.4 Hz, 1H, CH). MS (ESI; +): 618 (M + 1). | 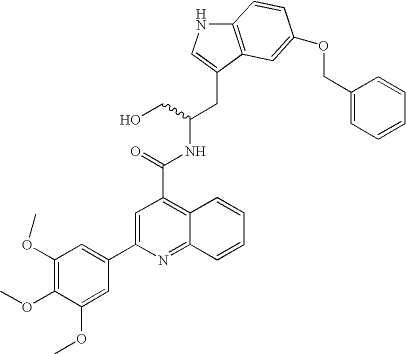 | | | |
| 10 | N-[(R,S)-1-(Hydroxymethyl)-2- (7-methyl-1H-indol-3-yl)ethyl]-2- (3,4,5-trimethoxyphenyl)- quinoline-4-carboxamide; (R,S)-β-Amino-7-methyl-1H- indole-3-propanol and 2-(3,4,5- Trimethoxyphenyl)quinoline-4- carboxylic acid | 1 | (DMSO-d₆): 10.82 s (1H, NH); 8.65 d (J=8.3 Hz, 1H, NH); 8.07 d (J=8.4 Hz, 1H, aryl); 8.02 s (1H, aryl); 7.83 d (J=8.3 Hz, 1H, aryl); 7.76 t (J=7.5 Hz, 1H, aryl); 7.54 s (2H, | |
| # aryl); 7.48 m (2H, aryl); 7.18 s (1H, aryl); 6.86 m (2H, aryl); 4.90 t (J=5.6 Hz, 1H, OH); 4.39 m (1H, OH); 3.93 s (6H, OCH₃); 3.76 s (3H, OCH₃); 3.58 m (2H, OCH₂); 3.08 dd (J=14.4 Hz/5.6 Hz, 1H, OH); 2.93 dd (J=14.4 Hz/8.2 Hz, 1H, CH); 2.46 s (3H, CH₃). MS (ESI; +): 526 (M + 1). | 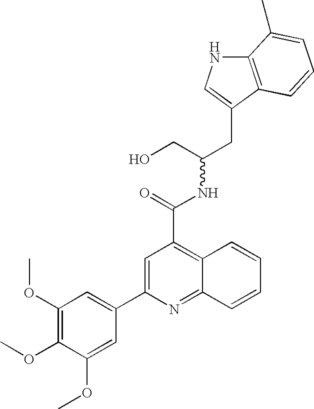 | | | |
| 11 | N-[(R)-1-(Hydroxymethyl)-2-(1H- indol-3-yl)ethyl]-2-(3,4,5-tri- methoxyphenyl)quinoline-4- carboxamide; D-Tryptophanol and 2-(3,4,5-Trimethoxyphenyl)- quinoline-4-carboxylic acid | 1e | (CDCl₃): 8.27 s (1H, NH); 8.13 d (J=8.4 Hz, 1H, aryl); 7.85 d (J=8.0 Hz, 1H, aryl); 7.72 d (J=8.0 Hz, 1H, aryl); 7.70 s (1H, aryl); 7.69 dd (J=8.4 Hz/8.0 Hz, 1H, aryl); 7.38 m (2H, aryl); 7.29 s (2H, aryl); 7.19 dd | |
| # (J=8.0 Hz/8.0 Hz, 1H, aryl); 7.11 d (J=8.0 Hz, 1H, aryl); 7.08 d (J=2.6 Hz, 1H, aryl); 6.51 d (J=8.0 Hz, 1H, NH); 4.66 m (1H, CH); 3.94 s (6H, OCH₃); 3.90 s (3H, OCH₃); 3.94 m (1H, CH₂OH); 3.81 dd (J=11.0 Hz/5.1 Hz, 1H, CH₂OH); 3.19 d (J=7.2 Hz, 2H, CH₂). | 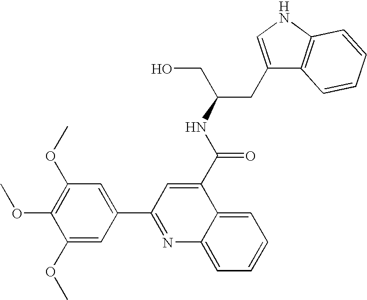 | | | |
| 12 | N-[(S)-1-(Hydroxymethyl)-2-(1H- indol-3-yl)ethyl]-2-(3,4,5- trimethoxyphenyl)quinoline-4- carboxamide; L-Tryptophanol and 2-(3,4,5-Trimethoxyphenyl)- quinoline-4-carboxylic acid | 1e | (CDCl₃): 8.21 s (1H, NH); 8.14 d (J=8.4 Hz, 1H, aryl); 7.86 d (J=8.0 Hz, 1H, aryl); 7.72 d (J=8.0 Hz, 1H, aryl); 7.71 s (1H, aryl); 7.69 dd (J=8.4 Hz/8.0 Hz, 1H, aryl); 7.38 m (2H, aryl); 7.29 s (2H, aryl); 7.20 dd (J=8.0 | |
| # Hz/8.0 Hz, 1H, aryl); 7.12 d (J=8.0 Hz, 1H, aryl); 7.09 d (J=2.6 Hz, 1H, aryl); 6.47 d (J=7.6 Hz, 1H, NH); 4.67 m (1H, CH); 3.95 s (6H, OCH₃); 3.91 s (3H, OCH₃); 3.93 m (1H, CH₂OH); 3.83 dd (J=11.0 Hz/5.1 Hz, 1H, CH₂OH); 3.20 d (J=7.2 Hz, 2H, CH₂). MS (ESI; +): 512 (M + 1). [α]_{D} = −16.5° (c = 0.475, MeOH/CH₂Cl₂ 1:1). | 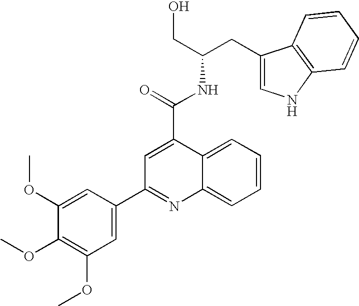 | | | |

### EXAMPLE 13

### 2-(4-Chloro-3-methylphenyl)-N-[(R)-1-(hydroxymethyl)-2-(1H-indol-3-yl)ethyl]quinoline-4-carboxamide

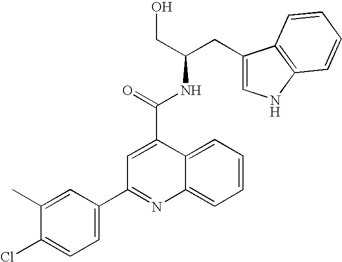

### 13a) 2-(4-Chloro-3-methylphenyl)quinoline-4-carboxylic acid

2.05 mmol (135 mg) of potassium hydroxide were slowly added to a stirred solution of 0.68 mmol (100 mg) of isatin in 7 ml of ethanol and 0.82 mmol (138 mg) of 4-chloro-3-methylacetophenone. After the addition was complete, the mixture was stirred at 80° C. for six hours. The solution was cooled and then the ethanol was removed in vacuo. The residue was taken up in water and acidified with 2 ml of 1°M aqueous hydrochloric acid. The aqueous phase was extracted with ethyl acetate. The resulting organic phase was dried over magnesium sulphate, filtered and concentrated in vacuo. Flash chromatography resulted in 145 mg of the target compound.

¹H-NMR (400 MHz, pyridine-d₅): δ [ppm]=9.33 d (J=8 Hz, 1H, aryl); 8.81 s (1H, aryl); 8.41 d (J=8 Hz, 1H, aryl); 8.28 s (1H, aryl); 8.15 dbr (J=8 Hz, 1H, aryl); 7.75 dd (J=8 Hz/7 Hz, 1H, aryl); 7.56 m (1H, aryl); 7.51 m (1H, aryl); 2.34 s (3H, Me).

### 13b) 2-(4-Chloro-3-methylphenyl)-N-[(R)-1-(hydroxymethyl)-2-(1H-indol-3-yl)ethyl]quinoline-4-carboxamide

In analogy to Example 1e), 67 mg of the title compound were obtained from 0.31 mmol (93 mg) of 2-(4-chloro-3-methylphenyl)quinoline-4-carboxylic acid and 0.26 mmol (50 mg) of D-tryptophanol.

¹H-NMR (400 MHz, pyridine-d₅): δ [ppm]=11.98 s (1H, NH); 9.65 d (J=8.4 Hz, 1H, NH); 8.54 d (J=7.6 Hz, 1H, aryl); 8.31 d (J=8.4 Hz, 1H, aryl); 8.20 d (J=7.6 Hz, 1H, aryl); 8.16 s (1H, aryl); 8.06 s (1H, aryl); 7.93 dd (J=8.4 Hz/2.1 Hz, 1H, aryl); 7.68 dd (J=8.0 Hz/7.6 Hz, 1H, aryl); 7.65 d (J=8.4 Hz, 1H, aryl); 7.56 s (1H, aryl); 7.48 d (J=8.4 Hz, 1H, aryl); 7.45 dd (J=8.4 Hz/8.0 Hz, 1H, aryl); 7.33 dd (J=8.0 Hz/7.6 Hz, 1H, aryl); 7.25 dd (J=8.4 Hz/8.0 Hz, 1H, aryl); 5.38 m (1H, CH); 4.38 dd (J=10.5 Hz/4.6 Hz, 1H, CH₂OH); 4.33 dd (J=10.5 Hz/5.5 Hz, 1H, CH₂OH); 3.73 dd (J=14.3 Hz/6.7 Hz, 1H, CH₂); 3.68 dd (J=14.3 Hz/6.7 Hz, 1H, CH₂); 2.31 s (3H, CH₃).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 14 | N-[(R)-1-(Hydroxymethyl)-2-(1H- indol-3-yl)ethyl]-6-methoxy-2- (3,4,5-trimethoxyphenyl)- quinoline-4-carboxamide; D-Tryptophanol and 6-Methoxy-2-(3,4,5- trimethoxyphenyl)quinoline-4- carboxylic acid | 13 | (CDCl₃): 8.19 s (1H); 8.04 d (J=8.4 Hz, 1H); 7.71 d (J=8.0 Hz, 1H); 7.68 s (1H); 7.38 m (3H); 7.20 m (1H); 7.11 m (2H); 6.52 d (J=8.0 Hz, 1H); 4.71 m (1H); 3.99 s (6H); 3.91 s (3H); 3.91 m (1H); | |
| # 3.82 m (1H); 3.72 s (3H); 3.22 m (2H); 2.62 t (J=5.7 Hz, 1H). MS (ESI; +): 542 (M + 1). | 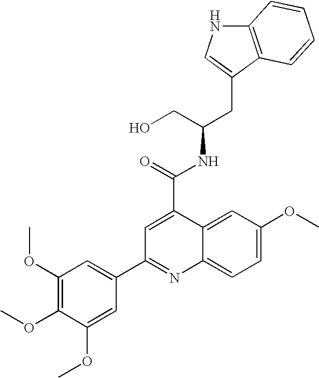 | | | |
| 15 | 6-Bromo-N-[(R)-1-(hydroxy- methyl)-2-(1H-indol-3-yl)ethyl]-2- (3,4,5- trimethoxyphenyl)quinoline-4- carboxamide; D-Tryptophanol und 6-Bromo-2-(3,4,5- trimethoxyphenyl)quinoline-4- carboxylic acid | 13 | (DMSO-d₆): 10.86 s (1H); 8.79 d (J=8.4 Hz, 1H); 8.25 d (J=2.1 Hz, 1H); 8.07 m (2H); 7.92 dd (J=2.1 Hz/8.9 Hz, 1H); 7.69 d (J=8.0 Hz, 1H); 7.58 s (2H); 7.35 d (J=8.0 Hz, 1H); 7.25 d (J=2.1 | |
| # Hz, 1H); 7.06 m (1H); 6.96 m (1H); 4.92 m (1H); 4.39 m (1H); 3.92 s (6H); 3.79 s (3H); 3.68 m (2H); 3.10 dd (J=6.3 Hz/14.8 Hz, 1H); 2.98 dd (J=7.6 Hz/14.3 Hz, 1H). MS (ESI; +): 591 (M + 1) | 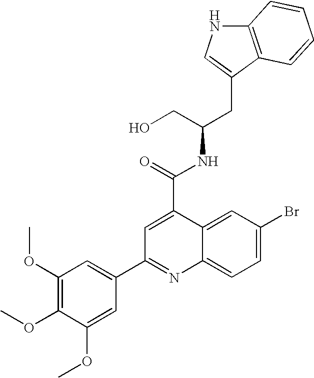 | | | |
| 16 | N-[(R)-1-(Hydroxymethyl)-2-(1H- indol-3-yl)ethyl]-6-methoxy-2- (2,3,4-trimethoxyphenyl)- quinoline-4-carboxamide; D-Tryptophanol and 6-Methoxy-2-(2,3,4- trimethoxyphenyl)quinoline-4- carboxylic acid | 13 | (CDCl₃): 8.23 s (1H); 8.03 d (J=9.1 Hz, 1H); 7.81 s (1H); 7.70 d (J=7.8 Hz, 1H); 7.58 m (2H); 7.37 m (2H); 7.12 m (3H); 6.80 d (J=8.8 Hz, 1H); 6.51 d (J=7.8 Hz, 1H); 4.61 m (1H); 3.98 s (3H); 3.95 | |
| # s (3H); 3.84 s (3H); 3.69 s (3H); 3.20 d (J=7.1 Hz, 2H); 2.68 s (1H). MS (ESI; +): 542 (M + 1). | 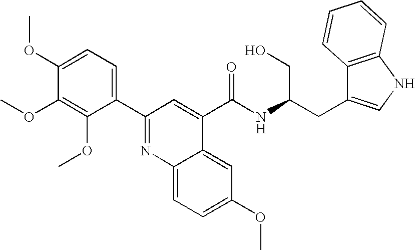 | | | |
| 17 | 6-Fluoro-N-[(R)-1-(hydroxy- methyl)-2-(1H-indol-3-yl)ethyl]-2- (3,4,5- trimethoxyphenyl)quinoline-4- carboxamide; D-Tryptophanol and 6-Fluoro-2-(3,4,5-trimethoxy- phenyl)quinoline-4-carboxylic acid | 13 | (CDCl₃): 8.22 s (1H); 8.15 m (1H); 7.71 m (2H); 7.63 dd (J=2.8 Hz/9.9 Hz, 1H); 7.48 m (1H); 7.39 d (J=8.1 Hz, 1H); 7.20 m (1H); 7.11 m (2H); 6.50 d (J=7.6 Hz, 1H); 4.69 m (1H); 3.99 s (6H); 3.91 | |
| # s (3H); 3.85 m (1H); 3.21 d (J=7.1 Hz, 2H); 2.60 s (1H). MS (ESI; +): 530 (M + 1). | 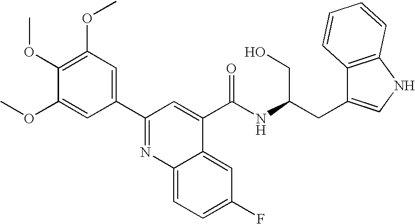 | | | |
| 18 | N-[(R)-1-(Hydroxymethyl)-2-(1H- indol-3-yl)ethyl]-6-iod-2-(3,4,5- trimethoxyphenyl)quinoline-4- carboxamide; D-Tryptophanol and 6-Iodo-2-(3,4,5- trimethoxyphenyl)quinoline-4- carboxylic acid | 13 | (DMSO-d₆): 10.82 s (1H); 8.73 d (J=8.3 Hz, 1H); 8.45 s (1H); 8.02 m (2H); 7.88 d (J=8.8 Hz, 1H); 7.64 d (J=7.8 Hz, 1H); 7.52 s (2H); 7.31 d (J=8.3 Hz, 1H); 7.03 m (1H); 6.92 m (1H); 5.72 s (1H); 4.88 m | |
| # (1H); 4.32 m (1H); 3.90 s (6H); 3.71 s (3H); 3.58 m (2H); 3.04 m (1H); 2.93 m (1H). MS (ESI; +): 638 (M + 1). | 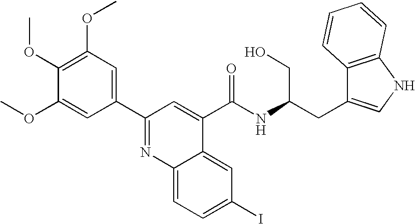 | | | |
| 19 | N-[(R)-1-(Hydroxymethyl)-2-(1H- indol-3-yl)ethyl]-6-nitro-2-(3,4,5- trimethoxyphenyl)quinoline-4- carboxamide; D-Tryptophanol and 6-Nitro-2-(3,4,5-trimethoxy- phenyl)quinoline-4-carboxylic acid | 13 | (DMSO-d₆): 10.80 s (1H); 8.98 d (J=2.6 Hz, 1H); 8.84 d (J=8.5 Hz, 1H); 8.46 dd (J=2.6 Hz/9.4 Hz, 1H); 8.28 m (2H); 7.64 s (1H); 7.61 s (2H); 7.29 d (J=7.9 Hz, 1H); 7.21 d (J=2.1 Hz, 1H); 6.99 | |
| # m (1H); 6.88 m (1H); 4.90 m (1H); 4.35 m (1H); 3.92 s (6H); 3.73 s (3H); 3.60 s (2H); 3.09 dd (J=6.2 Hz/14.7 Hz, 1H); 3.95 dd (J=7.7 Hz/14.5 Hz, 1H). MS (ESI; +): 557 (M + 1). | 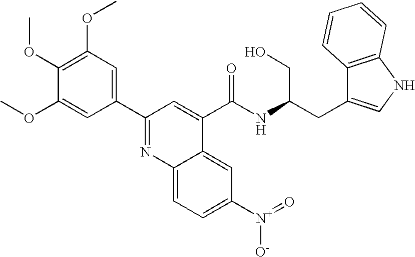 | | | |

### EXAMPLE 20

### 6-Amino-N-[(R)-1-(hydroxymethyl)-2-(1H-indol-3-yl)ethyl]-2-(3,4,5-trimethoxyphenyl)quinoline-4-carboxamide

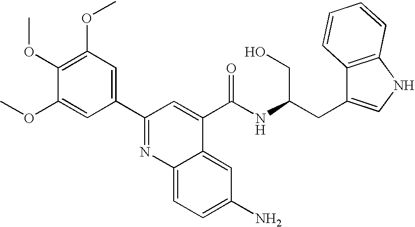

5.21 mmol (2.9 g) of the compound prepared in Example 19), and the catalyst palladium on carbon (10%, 500 mg) were suspended in methanol (40 ml) and hydrogenated with hydrogen under atmospheric pressure and at room temperature. After hydrogen uptake was complete, the catalyst was filtered off and the solvent was distilled off in a rotary evaporator. Oil-pump drying resulted in 2.15 g (78% yield) of the crystalline title compound.

¹H-NMR (400 MHz, DMSO-d₆): δ [ppm]=10.81 s (1H); 8.48 d (J=8.1 Hz, 1H); 7.74 m (2H); 7.68 d (J=7.8 Hz, 1H); 7.40 s (2H); 7.31 d (J=8.1 Hz, 1H); 7.21 d (J=2.3 Hz, 1H); 7.13 dd (J=2.5 Hz/9.1 Hz, 1H); 7.03 m (2H); 6.98 m (1H); 5.70 s (2H); 4.82 m (1H); 4.29 m (1H); 3.88 s (6H); 3.70 s (3H); 3.58 m (1H); 3.51 m (1H); 3.06 dd (J=6.6 Hz/14.7 Hz, 1H); 2.93 dd (J=7.6 Hz/14.7 Hz, 1H).

MS (ESI; +): 527 (M+1).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 21 | N-[(R,S)-1-(Hydroxymethyl)-2- (5-fluoro-1H-indol-3-yl)ethyl]-6- methoxy-2-(3,4,5-trimethoxy- phenyl)quinoline-4- carboxamide; (R,S)-β-Amino-5-fluoro-1H- indole-3-propanol and 6-Methoxy-2-(3,4,5-trimethoxy- phenyl)quinoline-4-carboxylic acid | 1 13 | (DMSO-d₆): 10.94 s (1H, NH); 8.71 d (J=8.4 Hz, 1H, NH); 8.00 d (J=8.6 Hz, 1H, aryl); 7.97 s (1H, aryl); 7.50 s (2H, aryl); 7.41 m (3H, aryl); 7.32 | |
| # m (1H, aryl); 7.29 m (1H, aryl); 6.88 t (J=8.9 Hz, 1H, aryl); 4.96 s (1H, OH); 4.37 m (1H, CH); 3.93 s (6H, OCH₃); 3.75 s (6H, OCH₃); 3.60 m (2H, OCH₂); 3.01 dd (J=14.4 Hz/5.6 Hz, 1H, CH); 2.91 dd (J=14.4 Hz/7.8 Hz, 1H, CH). ¹⁹F-NMR (400 MHz, DMSO-d₆): −124.81 m (1F). MS (ESI; +): 560 (M + 1). | 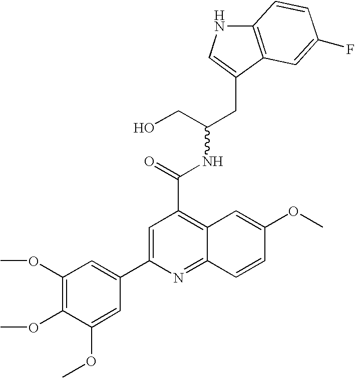 | | | |
| 22 | N-[(R)-1-(Hydroxymethyl)-2-(1- methyl-1H-indol-3-yl)ethyl]-6- methoxy-2-(3,4,5-trimethoxy- phenyl)quinoline-4-carboxamide (R)-β-Amino-1-methyl-1H- indole-3-propanol and 6-Methoxy-2-(3,4,5-trimethoxy- phenyl)quinoline-4-carboxylic acid | 1 13 | (DMSO-d₆): 8.67 d (J=8.3 Hz, 1H, NH); 8.00 d (J=7.8 Hz, 1H, aryl); 7.99 s (1H, aryl); 7.68 d (J=7.9 Hz, 1H, aryl); 7.51 s (2H, aryl); 7.41 m (3H, aryl); 7.18 | |
| # s (1H, aryl); 7.12 t (J=7.5 Hz, 1H, aryl); 6.99 t (J=7.4 Hz, 1H, aryl); 4.91 t (J=5.3 Hz, 1H, OH); 4.38 m (1H, CH); 3.93 s (6H, OCH₃); 3.75 s (6H, OCH₃); 3.72 s (3H, NCH₃); 3.60 t (J=5.2 Hz, 2H, OCH₂); 3.03 dd (J=14.4 Hz/6.1 Hz, 1H, CH); 2.96 dd (J=14.4 Hz/7.5 Hz, 1H, CH). MS (APCI; +): 556 (M + 1). | 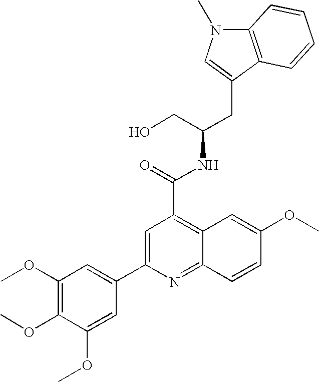 | | | |
| 23 | N-[(R,S)-2-(6-Fluoro-1H-indol-3- yl)-1-(hydroxymethyl)ethyl]-6- methoxy-2-(3,4,5-trimethoxy- phenyl)quinoline-4- carboxamide; (R,S)-β-Amino-6-fluoro-1H- indole-3-propanol and 6-Methoxy-2-(3,4,5-trimethoxy- phenyl)quinoline-4-carboxylic acid | 1 13 | (DMSO-d₆): 10.90 s (1H, NH); 8.67 d (J=8.4 Hz, 1H, NH); 8.00 dd (J=8.6 Hz, 1H, aryl); 7.97 s (1H, aryl); 7.63 m (1H, aryl); 7.50 s (2H, aryl); 7.42 m (2H, | |
| # aryl); 7.22 s (1H, aryl); 7.10 d (J=10.2 Hz, 1H, aryl); 6.81 t (J=8.1 Hz, 1H, aryl); 4.92 t (J=5.4 Hz, 1H, OH); 4.39 m (1H, CH); 3.60 t (J=5.3 Hz, 2H, OCH₂); 3.04 dd (J=14.5 Hz/5.8 Hz, 1H, CH); 2.93 dd (J=14.6 Hz/7.9 Hz, 1H, CH). ¹⁹F-NMR (400 MHz, DMSO-d₆): −121.70 m (1F). MS (APCI; +): 560 (M + 1). | 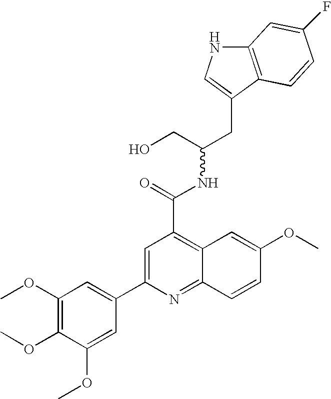 | | | |
| 24 | 2-(3,4-Dimethoxyphenyl)-N-[(S)- 1-(hydroxymethyl)-2-(1H-indol-3- yl)ethyl]quinoline-4- carboxamide; L-Tryptophanol and 2-(3,4-Dimethoxyphenyl)- quinoline-4-carboxylic acid | 13 | (Pyridin-d₅): 11.97 s (1H, NH); 9.63 d (J=8.4 Hz, 1H, NH); 8.53 d (J=7.6 Hz, 1H, aryl); 8.32 d (J=8.4 Hz, 1H, aryl); 8.28 s (1H, aryl); 8.20 d (J=7.6 Hz, 1H, aryl); 8.13 d (J=2.1 Hz, 1H, aryl); 7.70 dd (J=8.4 Hz/2.1 Hz, | |
| # 1H, aryl); 7.65 dd (J=8.0 Hz/7.6 Hz, 1H, aryl); 7.64 d (J=8.0 Hz, 1H, aryl); 7.58 d (J=2.1 Hz, 1H, aryl); 7.41 dd (J=8.4 Hz/8.0 Hz, 1H, aryl); 7.33 dd (J=7.6 Hz/7.0 Hz, 1H, aryl); 7.26 dd (J=8.0 Hz/7.0 Hz, 1H, aryl); 7.00 d (J=8.4 Hz, 1H, aryl); 5.38 m (1H, CH); 4.35 m (2H, CH₂OH); 3.78 s (3H, OCH₃); 3.76 s (3H, OCH₃); 3.70 m (2H, CH₂). [α]_{D} = −11.0° | | | | |
| # (c = 0.330, MeOH/CH₂Cl₂ 1:1) | 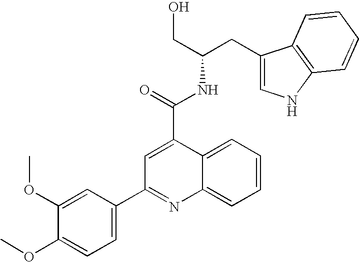 | | | |
| 25 | 2-(3,4-Dimethoxyphenyl)-N-[(R)- 1-(hydroxymethyl)-2-(1H-indol-3- yl)ethyl]quinoline-4- carboxamide; D-Tryptophanol and 2-(3,4-Dimethoxyphenyl)- quinoline-4-carboxylic acid | 13 | (Pyridin-d₅): 11.97 s (1H, NH); 9.63 d (J=8.4 Hz, 1H, NH); 8.53 d (J=7.6 Hz, 1H, aryl); 8.32 d (J=8.4 Hz, 1H, aryl); 8.28 s (1H, aryl); 8.20 d (J=7.6 Hz, 1H, aryl); 8.13 d (J=2.1 Hz, 1H, aryl); 7.70 dd (J=8.4 Hz/2.1 Hz, | |
| # 1H, aryl); 7.65 dd (J=8.0 Hz/7.6 Hz, 1H, aryl); 7.64 d (J=8.4 Hz, 1H, aryl); 7.58 d (J=2.1 Hz, 1H, aryl); 7.41 dd (J=8.4 Hz/8.0 Hz, 1H, aryl); 7.33 dd (J=8.4 Hz/8.0 Hz, 1H, aryl); 7.26 dd (J=8.0 Hz/7.6 Hz, 1H, aryl); 7.00 d (J=8.4 Hz, 1H, aryl); 5.38 m (1H, CH); 4.35 m (2H, CH₂OH); 3.78 s (3H, OCH₃); 3.76 s (3H, OCH₃); 3.70 m (2H, CH₂). [α]_{D} = +12.1° | | | | |
| # (c = 0.550, MeOH/CH₂Cl₂ 1:1) | 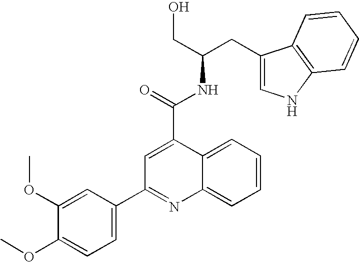 | | | |
| 26 | 2-(3,4-Dimethoxyphenyl)-N-[(R)-1- (hydroxymethyl)-2-(1H-indol-3- yl)ethyl]quinoline-4- carboxamide; D-Tryptophanol and 2-(3,4-Dimethoxyphenyl)quinoline- 4-carboxylic acid | 13 | (CDCl₃): 8.15 s (1H, NH); 8.13 d (J=8.0 Hz, 1H, aryl); 7.93 d (J=8.4 Hz, 1H, aryl); 7.89 s (1H, aryl); 7.74 d (J=8.0 Hz, 1H, aryl); 7.69 dd (J=8.0 Hz/8.0 Hz, 1H, aryl); 7.68 d (J=8.0 Hz, 1H, aryl); 7.65 s (1H, aryl); 7.39 dd | |
| # (J=8.0 Hz/8.0 Hz, 2H, aryl); 7.25 d (J=8.4 Hz, 1H, aryl); 7.23 dd (J=8.0 Hz/8.0 Hz, 1H, aryl); 7.15 d(J=8.0 Hz, 1H, aryl); 7.12 s (1H, aryl); 6.43 d (J=7.6 Hz, 1H, NH); 4.66 m (1H, CH); 3.93 d (J=11.0 Hz, 1H, CH₂OH); 3.85 dd (J=11.0 Hz/5.0 Hz, 1H, CH₂OH); 3.22 d (J=7.2 Hz, 2H, CH₂); 2.38 s (3H, CH₃); 2.35 s (3H, CH₃). | 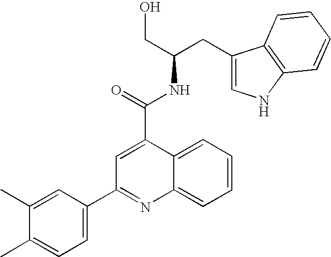 | | | |
| 27 | 2-(2,3-Dihydro-1,4-benzodioxin- 6-yl)-N-[(R)-1-(hydroxymethyl)- 2-(1H-indol-3-yl)ethyl]quinoline- 4-carboxamide; D-Tryptophanol and 2-(2,3-Dihydro-1,4-benzodioxin- 6-yl)quinoline-4-carboxylic acid | 13 | (CDCl₃): 8.22 s (1H, NH); 8.06 d (J=8.4 Hz, 1H, aryl); 7.92 d (J=8.0 Hz, 1H, aryl); 7.72 d (J=8.0 Hz, 1H, aryl); 7.65 dd (J=8.4 Hz/8.0 Hz, 1H, aryl); 7.58 d (J=2.1 Hz, 1H, aryl); 7.46 dd (J=8.4 | |
| # Hz/2.1 Hz, 1H, aryl); 7.44 s (1H, aryl); 7.38 dd (J=8.0 Hz/8.0 Hz, 1H, aryl); 7.36 dd (J=8.0 Hz/8.0 Hz, 1H, aryl); 7.22 dd (J=8.0 Hz/8.0 Hz, 1H, aryl); 7.15 d (J=8.0 Hz, 1H, aryl); 7.11 d (J=2.5 Hz, 1H, aryl); 6.96 d (J=8.4 Hz, 1H, aryl); 6.43 d (J=7.6 Hz, 1H, NH); 4.63 m (1H, CH); 4.32 s (4H, CH₂O); 3.93 dd (J=11.0 Hz/3.8 Hz, 1H, CH₂OH); 3.83 dd (J=11.0 Hz/5.5 Hz, 1H, | | | | |
| # CH₂OH); 3.22 dd (J=15.0 Hz/8.0 Hz, 1H, CH₂); 3.17 dd (J=15.0 Hz/6.7 Hz, 1H, CH₂). | 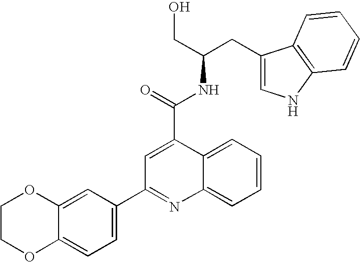 | | | |
| 28 | N-[(R)-1-(Hydroxymethyl)-2-(1H- indol-3-yl)ethyl]-2-[4- (trifluoromethoxy)phenyl]- quinoline-4-carboxamide D-Tryptophanol 2-[4-(Trifluoromethoxy)phenyl]- quinoline-4-carboxylic acid | 13 | (Pyridin-d₅): 11.99 s (1H, NH); 9.60 d (J=8.0 Hz, 1H, NH); 8.52 d (J=7.6 Hz, 1H, aryl); 8.28 d (J=8.4 Hz, 1H, aryl); 8.18 d (J=7.6 Hz, 1H, aryl); 8.15 d (J=8.9 Hz, 1H, aryl); 8.10 s (1H, aryl); 7.67 dd (J= | |
| # 8.0 Hz/7.6 Hz, 1H, aryl); 7.65 d (J=8.4 Hz, 1H, aryl); 7.57 d (J=2.5 Hz, 1H, aryl); 7.44 dd (J=8.4 Hz/8.0 Hz, 1H, aryl); 7.36 d (J=8.9 Hz, 1H, aryl); 7.33 dd (J=8.0 Hz/7.6 Hz, 1H, aryl); 7.24 dd (J=8.4 Hz/8.0 Hz, 1H, aryl); 5.38 m (1H, CH); 4.38 dd (J=10.5 Hz/5.1 Hz, 1H, CH₂OH); 4.32 dd (J=10.5 Hz/5.5 Hz, 1H, CH₂OH); 3.72 dd (J=16.4 Hz/6.7 Hz, 1H, CH₂); 3.68 dd (J=16.4 | | | | |
| # Hz/6.7 Hz, 1H, CH₂). | 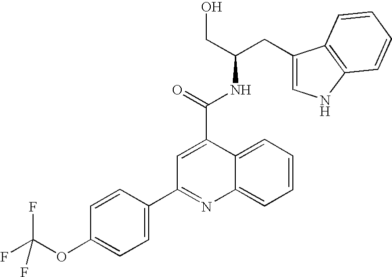 | | | |
| 29 | N-[(R)-1-(Hydroxymethyl)-2-(1H- indol-3-yl)ethyl]-2-[4- (methylsulphanyl)phenyl]- quinoline-4-carboxamide; D-Tryptophanol and (Methylsulphanyl)phenyl]- quinoline-4-carboxylic acid | 13 | (CDCl₃): 8.16 s (1H, NH); 8.11 d (J=8.4 Hz, 1H, aryl); 7.93 d (J=8.6 Hz, 1H, aryl); 7.93 d (J=8.0 Hz, 1H, aryl); 7.73 d (J=8.0 Hz, 1H, aryl); 7.56 s (1H, aryl); 7.69 dd (J=8.4 Hz/8.0 Hz, 1H, aryl); 7.41 d | |
| # (J=8.0 Hz, 1H, aryl); 7.40 dd (J=8.0 Hz/8.0 Hz, 1H, aryl); 7.35 d (J=8.6 Hz, 1H, aryl); 7.24 dd (J=8.0 Hz/8.0 Hz, 1H, aryl); 7.14 dd (J=8.0 Hz/8.0 Hz, 1H, aryl); 7.12 s (1H, aryl); 6.40 d (J=7.6 Hz, 1H, NH); 4.66 m (1H, CH); 3.94 dd (J=11.4 Hz/3.8 Hz, 1H, CH₂OH); 3.85 dd (J=11.4 Hz/5.5 Hz, 1H, CH₂OH); 3.23 dd (J=15.6 Hz/7.2 Hz, 1H, CH₂); 3.20 dd (J=15.6 Hz/7.2 Hz, 1H, | | | | |
| # CH₂); 2.56 s (3H, SCH₃). | 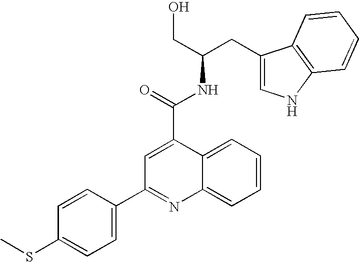 | | | |
| 30 | 2-(3,5-Dimethoxyphenyl)-N-[(R)- 1-(hydroxymethyl)-2-(1H-indol-3- yl)ethyl]quinoline-4-carboxamide D-Tryptophanol 2-(3,5- Dimethoxyphenyl)quinoline-4- carboxylic acid | 13 | (Pyridine-d₅): 11.96 s (1H, NH); 9.66 d (J=8.0 Hz, 1H, NH); 8.52 d (J=7.6 Hz, 1H, aryl); 8.32 d (J=8.4 Hz, 1H, aryl); 8.32 s (1H, aryl); 8.20 d (J=7.6 Hz, 1H, aryl); 7.66 dd (J=8.0 Hz/7.6 Hz, 1H, aryl); 7.63 d (J=8.4 Hz, 1H, | |
| # aryl); 7.58 d (J=2.3 Hz, 2H, aryl); 7.57 s (1H, aryl); 7.43 dd (J=8.4 Hz/8.0 Hz, 1H, aryl); 7.32 dd (J=8.4 Hz/8.0 Hz, 1H, aryl); 7.26 dd (J=8.0 Hz/7.6 Hz, 1H, aryl); 6.78 t (J=2.3 Hz, 1H, aryl); 5.37 m (1H, CH); 4.34 m (2H, CH₂OH); 3.71 s (6H, OCH₃); 3.69 m (2H, CH₂). | 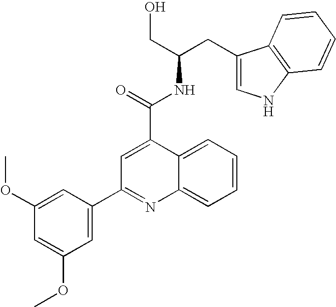 | | | |
| 31 | 2-[3-(Acetylamino)phenyl]-N- [(R)-1-(hydroxymethyl)-2-(1H- indol-3-yl)ethyl]quinoline-4- carboxamide; D-Tryptophanol and 2-[4-(Acetylamino)phenyl]- quinoline-4-carboxylic acid | 13 | (Pyridine-d₅): 11.91 s (1H, NH); 10.91 s (1H, NH); 9.57 d (J=8.4 Hz, 1H, NH); 8.90 s (1H, aryl); 8.51 d (J=7.6 Hz, 1H, aryl); 8.21 d (J=8.4 Hz, 1H, aryl); 8.20 s (1H, aryl); 8.19 d (J=7.6 Hz, 1H, aryl); 8.15 d | |
| # (J=8.0 Hz, 1H, aryl); 7.82 d (J=8.0 Hz, 1H, aryl); 7.65 d (J=8.4 Hz, 1H, aryl); 7.64 dd (J=8.0 Hz/7.6 Hz, 1H, aryl); 7.63 s (1H, aryl); 7.43 dd (J=8.4 Hz/8.0 Hz, 1H, aryl); 7.41 dd (J=8.0 Hz/8.0 Hz, 1H, aryl); 7.33 dd (J=8.4 Hz/8.0 Hz, 1H, aryl); 7.26 dd (J=8.0 Hz/7.6 Hz, 1H, aryl); 5.36 m (1H, CH); 4.36 m (2H, CH₂OH); 3.71 s (6H, CH₂OH); 3.73 dd (J=14.6 Hz/6.7 Hz, 1H, CH₂); | | | | |
| # 3.68 dd (J=14.6 Hz/7.2 Hz, 1H, CH₂); 2.24 s (3H, CH₃). | 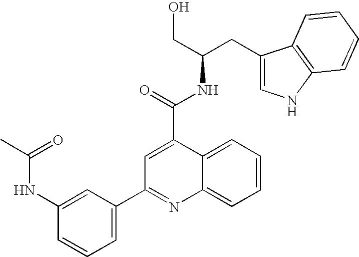 | | | |
| 32 | 2-(4-Chlorophenyl)-N-[(R)-1- (hydroxymethyl)-2-(1H-indol-3- yl)ethyl]quinoline-4- carboxamide; D-Tryptophanol and 2-(4-Chlorophenyl)quinoline-4- carboxylic acid | 13 | (CDCl₃): 8.14 s (1H, NH); 8.13 d (J=8.4 Hz, 1H, aryl); 7.98 d (J=8.0 Hz, 1H, aryl); 7.95 d (J=8.6 Hz, 2H, aryl); 7.74 d (J=8.0 Hz, 1H, aryl); 7.72 dd (J=8.4 Hz/8.0 Hz, 1H, aryl); 7.56 s (1H, aryl); 7.47 d (J=8.6 Hz, 2H, aryl); | |
| # 7.45 dd (J=8.0 Hz/8.0 Hz, 1H, aryl); 7.44 d (J=8.0 Hz, 1H, aryl); 7.25 dd (J=8.0 Hz/8.0 Hz, 1H, aryl); 7.14 dd (J=8.0 Hz/8.0 Hz, 1H, aryl); 7.14 s (1H, aryl); 6.37 d (J=7.6 Hz, 1H, NH); 4.69 m (1H, CH); 3.96 d (J=11.4 Hz, 1H, CH₂OH); 3.86 d (J=11.4 Hz, 1H, CH₂OH); 3.26 dd (J=14.8 Hz/6.3 Hz, 1H, CH₂); 3.21 dd (J=14.8 Hz/7.6 Hz, 1H, CH₂). | 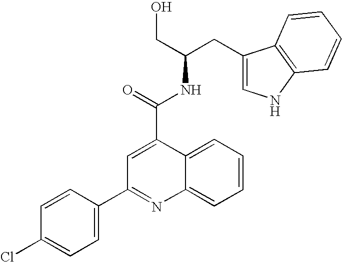 | | | |
| 33 | N-[(R)-1-(Hydroxymethyl)-2-(1H- indol-3-yl)ethyl]-2-(4-methoxy- phenyl)quinoline-4-carboxamide; D-Tryptophanol and 2-(4-Methoxyphenyl)quinoline-4- carboxylic acid | 13 | (CDCl₃): 8.16 s (1H, NH); 8.09 d (J=8.4 Hz, 1H, aryl); 7.94 d (J=8.9 Hz, 2H, aryl); 7.91 d (J=8.0 Hz, 1H, aryl); 7.73 d (J=8.0 Hz, 1H, aryl); 7.67 dd (J=8.4 Hz/8.0 Hz, 1H, aryl); 7.53 s (1H, aryl); 7.40 d (J=8.0 HZ, 1H, aryl); | |
| # 7.37 dd (J=8.0 Hz/8.0 Hz, 1H, aryl); 7.24 dd (J=8.0 Hz/8.0 Hz, 1H, aryl); 7.14 dd (J=8.0 Hz/8.0 Hz, 1H, aryl); 7.10 d (J=2.5 Hz, 1H, aryl); 7.00 d (J=8.9 Hz, 2H, aryl); 6.40 d (J=8.0 Hz, 1H, NH); 4.65 m (1H, CH); 3.92 d (J=11.4 Hz, 1H, CH₂OH); 3.89 s (3H, CH₃); 3.84 d (J=11.4 Hz, 1H, CH₂OH); 3.21 m (2H, CH₂). | 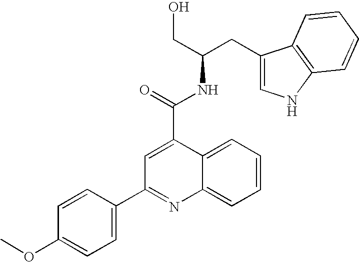 | | | |
| 34 | N-[(R)-1-(Hydroxymethyl)-2-(1H- indol-3-yl)ethyl]-2-(3- methoxyphenyl)quinoline-4- carboxamide D-Tryptophanol 2-(3-Methoxyphenyl)quinoline-4- carboxylic acid | 13 | (Pyridine-d₅): 11.97 s (1H, NH); 9.63 d (J=8.0 Hz, 1H, NH); 8.52 d (J=7.6 Hz, 1H, aryl); 8.31 d (J=8.4 Hz, 1H, aryl); 8.23 s (1H, aryl); 8.20 d (J=7.6 Hz, 1H, aryl); 8.03 dd (J=2.1 Hz/1.7 Hz, 1H, aryl); 7.67 dd (J=8.0 Hz/7.6 Hz, | |
| # 1H, aryl); 7.64 dd (J=8.0 Hz/1.7 Hz, 1H, aryl); 7.64 d (J=8.4 Hz, 1H, aryl); 7.56 s (1H, aryl); 7.43 dd (J=8.4 Hz/8.0 Hz, 1H, aryl); 7.39 dd (J=8.0 Hz/7.6 Hz, 1H, aryl); 7.34 dd (J=8.0 Hz/7.6 Hz, 1H, aryl); 7.26 dd (J=8.4 Hz/8.0 Hz, 1H, aryl); 7.08 dd (J=7.6 Hz/2.1 Hz Hz, 1H, aryl); 5.38 m (1H, CH); 4.36 m (2H, CH₂OH); 3.71 s (3H, OCH₃); 3.71 m (2H, CH₂). | 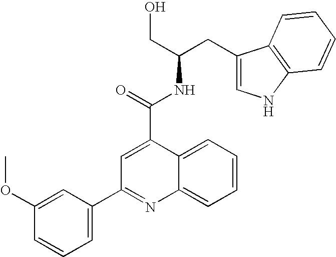 | | | |
| 35 | N-[(R)-2-(1-Ethyl-1H-indol-3-yl)- 1-(hydroxymethyl)ethyl]-6- methoxy-2-(3,4,5-trimethoxy- phenyl)quinoline-4- carboxamide; (R)-2-Amino-3-(1-ethyl-1H- indol-3-yl)-propan-1-ol and 6-Methoxy-2-(2,3,4-tri-methoxy- phenyl)quinoline-4-carboxylic acid | 13 | (DMSO-d₆): 8.69 d (J=8.3 Hz, 1H, NH), 8.00 d (J=8.3 Hz, 1H, aryl); 7.99 s (1H, aryl); 7.67 d (J=7.84, 1H, aryl); 7.50 s (2H, aryl); 7.44 m (3H, aryl); | |
| # 7.24 s (1H, aryl); 7.10 t (J=7.7 Hz, 1H, aryl); 6.97 t (J=7.3 Hz, 1H, aryl); 4.93 t (J=5.7 Hz, 1H, OH): 4.38 m =5.7 Hz, 1H, OH); 4.38 m (1H, CH); 4.13 q (J=7.17 Hz, 2H, NC₂H₅); 3.92 s (6H, OCH₃); 3.75 s (6H, OCH₃); 3.61 m (2H, OCH₂); 3.03 dd (J=14.4 Hz/5.8 Hz, 1H, CH); 2.95 dd (J=14.4 Hz/7.6 Hz, 1H, CH); 1.27 t (J=7.17 Hz, 3H, NC₂H₅). MS (APCI; +): | | | | |
| # 570 (M + 1) | 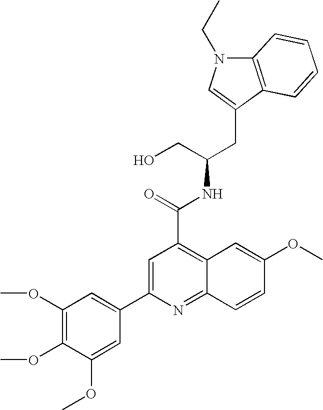 | | | |
| 36 | 2-(2,3-Dihydrobenzofuran-5-yl)- N-[(R)-1-(hydroxymethyl)-2-(1H- indol-3-yl)ethyl]quinoline-4- carboxamide; D-Tryptophanol and 2-(2,3-Dihydrobenzofuran-5- yl)quinoline-4-carboxylic acid | | (CDCl₃): 8.19 s (1H, NH); 8.06 d (J=8.3 Hz, 1H, aryl); 7.93 s (1H, aryl); 7.89 d (J=8.3 Hz, 1H, aryl); 7.72 d (J=7.8 Hz, 1H, aryl); 7.65 dd (J=8.3 Hz/8.3 Hz, 1H, aryl); 7.64 d (J=8.3 Hz, 1H, aryl); 7.49 s (1H, | |
| # aryl); 7.39 d (J=8.3 Hz, 1H, aryl); 7.35 dd (J=8.3 Hz/8.3 Hz, 1H, aryl); 7.22 dd (J=8.3 Hz/7.0 Hz, 1H, aryl); 7.13 dd (J=7.8 Hz/7.0 Hz, 1H, aryl); 7.08 d (J=2.3 Hz, 1H, aryl); 6.85 d (J=8.3 Hz, 1H, aryl); 6.44 d (J=7.8 Hz, 1H, NH); 4.63 m (1H, CH); 4.64 t (J=8.7 Hz, 2H, CH₂O); 3.90 dd (J=11.1 Hz/3.8 Hz, 1H, CH₂OH); 3.82 dd (J=11.1 Hz/5.1 Hz, 1H, CH₂OH); 3.27 t (J=8.7 | | | | |
| # Hz, 2H, CH₂); 3.21 dd (J=15.7 Hz/6.8 Hz, 1H, CH₂); 3.17 dd (J=15.7 Hz/6.8 Hz, 1H, CH₂). | 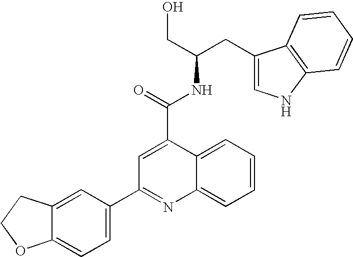 | | | |
| 37 | N-[(R)-1-(Hydroxymethyl)-2-(1H- indol-3-yl)ethyl]-6-methoxy-2-(7- methoxybenzofuran-2-yl)- quinoline-4-carboxamide; D-Tryptophanol and 6-Methoxy-2-(7-methoxy- benzofuran-2-yl)quinoline-4- carboxylic acid | | (Pyridine-d₆): 11.85 S (1H, NH); 9.64 d (J=8.3 Hz, 1H, NH); 8.45 s (1H, aryl); 8.20 d (J=9.2 Hz, 2H, aryl); 8.06 d (J=2.8 Hz, 1H, aryl); 7.68 s (1H, aryl); 7.62 s (1H, aryl); 7.59 d (J=8.0 Hz, | |
| # 1H, aryl); 7.44 dd (J=9.2 Hz/2.8 Hz, 1H, aryl); 7.33 d (J=7.8 Hz, 1H, aryl); 7.31 dd(J=8.0 Hz/8.0 Hz, 1H, aryl); 7.29 dd (J=9.2 Hz/8.0 Hz, 1H, aryl); 7.24 dd (J=7.8 Hz/7.8 Hz, 1H, aryl); 6.93 d (J=7.8 Hz, 1H, aryl); 5.37 m (1H, CH); 4.38 dd (J=10.9 Hz/4.5 Hz, 2H, CH₂OH); 4.30 dd (J=10.9 Hz/6.0 Hz, 2H, CH₂OH); 3.92 s (3H, OCH₃); 3.70 s (3H, OCH₃); 3.68 m (2H, CH₂). | | | | |
| # 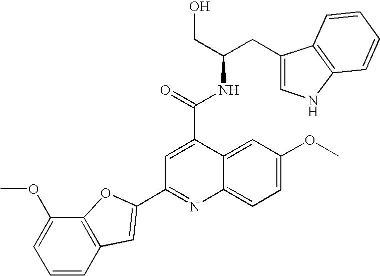 | | | | |
| 38 | 2-[(E)-2-(3,4-Dimethoxyphenyl)- ethenyl]-N-[(R)-1-(hydroxy- methyl)-2-(1H-indol-3-yl)ethyl]-6- methoxyquinoline-4- carboxamide; D-Tryptophanol and 2-[(E)-2-(3,4- Dimethoxyphenyl)ethenyl]-6- methoxyquinoline-4-carboxylic acid | 13 | (DMSO-d₆): 10.80 s (1H); 8.55 d (J=8.5 Hz, 1H); 7.85 d (J=9.6 Hz, 1H); 7.60 m (3H); 7.33 m (5H); 7.19 m (2H); 6.98 m (3H); 4.85 t (J=5.7 Hz, 1H); 4.38 m (1H); 3.84 s | |
| # (3H); 3.79 s (3H); 3.68 s (3H); 3.57 t (J=5.7 Hz, 1H); 3.02 dd (J=14.9 Hz/6.2 Hz, 1H); 2.91 m (1H). MS (ESI; +): 538 (M + 1). | 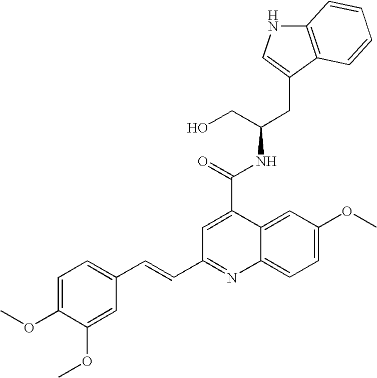 | | | |

### EXAMPLE 39

### N-[(R)-1-(Hydroxymethyl)-2-(1H-indol-3-yl)ethyl]-4′-methoxy[1,1′-biphenyl]-2-carboxamide

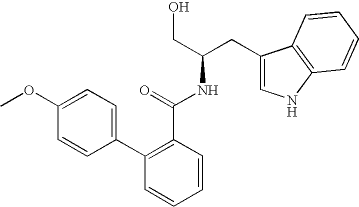

### 39a) ethyl 4′-methoxy[1,1′-biphenyl]-2-carboxylate

0.66 mmol (100 mg) of 4-methoxyphenylboronic acid, 0.88 mmol (0.88 ml) of a 1 molar solution of tetrabutylammonium fluoride in tetrahydrofuran and 0.044 mmol (51 mg) of tetrakis(triphenylphosphine)palladium(0) were added to a solution of 0.44 mmol (69 μl) of ethyl 2-bromobenzoate in 4.4 ml of toluene and 2.2 ml of ethanol. The mixture was heated to boiling for four hours. After cooling, the reaction mixture was diluted with saturated aqueous sodium hydrogen carbonate solution and extracted with ethyl acetate. The combined organic phases were washed with saturated aqueous sodium chloride solution, dried over sodium sulphate, filtered and concentrated in vacuo. Flash chromatography resulted in 107 mg of the target compound.

¹H-NMR (400 MHz, CDCl₃): δ [ppm]=7.79 d (J=7.8 Hz, 1H, aryl); 7.50 dd (J=7.6 Hz/7.3 Hz, 1H, aryl); 7.37 dd (J=7.8 Hz/7.3 Hz, 1H, aryl); 7.36 d (J=7.6 Hz, 1H, aryl); 7.26 d (J=8.6 Hz, 1H, aryl); 6.93 d (J=8.6 Hz, 1H, aryl); 4.12 q (J=7.1 Hz, 2H, OCH₂); 3.85 s (3H, OCH₃); 1.06 t (J=7.1 Hz, 3H, CH₃).

### 39b) 4′-methoxy[1,1′-biphenyl]-2-carboxylic acid

0.39 mmol (100 mg) of the compound prepared as in 39a) were stirred in 4 ml of methanol with 2.39 ml of a 2 molar aqueous sodium hydroxide solution at room temperature for 16 hours. The reaction mixture was concentrated in vacuo, acidified to pH 4 with 1°M aqueous hydrochloric acid and stirred for a further hour. 82 mg of the target compound were obtained by filtering off the precipitate with suction.

¹H-NMR (400 MHz, CDCl₃): δ [ppm]=7.92 d (J=7.8 Hz, 1H, aryl); 7.54 dd (J=7.6 Hz/7.6 Hz, 1H, aryl); 7.39 dd (J=7.8 Hz/7.6 Hz, 1H, aryl); 7.36 d (J=7.6 Hz, 1H, aryl); 7.27 d (J=8.7 Hz, 1H, aryl); 6.93 d (J=8.6 Hz, 1H, aryl); 3.85 s (3H, OCH₃).

### 39c) N-[(R)-1-(Hydroxymethyl)-2-(1H-indol-3-yl)ethyl]-4′-methoxy[1,1′-biphenyl]-2-carboxamide

In analogy to Example 1e), 89 mg of the title compound were obtained from 0.33 mmol (75 mg) of the compound prepared as in 39b) and 0.26 mmol (50 mg) of D-tryptophanol.

¹H-NMR (400 MHz, CDCl₃): δ [ppm]=8.05 s (1H, NH); 7.63 d (J=7.8 Hz, 1H, aryl); 7.54 d (J=7.8 Hz, 1H, aryl); 7.44 dd (J=7.8 Hz/7.5 Hz, 1H, aryl); 7.34 d (J=8.0 Hz, 1H, aryl); 7.33 dd (J=7.8 Hz/7.5 Hz, 1H, aryl); 7.31 d (J=7.8 Hz, 1H, aryl); 7.29 d (J=8.8 Hz, 2H, aryl); 7.19 dd (J=7.8 Hz/7.1 Hz, 1H, aryl); 7.09 dd (J=8.0 Hz/7.1 Hz, 1H, aryl); 6.89 d (J=8.8 Hz, 2H, aryl); 6.84 d (J=2.3 Hz, 1H, aryl); 5.61 d (J=7.6 Hz, 1H, NH); 4.26 m (1H, CH); 3.78 s (3H, OCH₃); 3.48 m (2H, CH₂OH); 2.77 d (J=6.6 Hz, 2H, CH₂).

The following compounds were obtained in analogy to the preparation methods described in detail:

| | | Method | | |
|---|---|---|---|---|
| | Product; | analogous | ¹H-NMR (400 MHz) δ5 | |
| Ex. | reagents | to | [ppm] | Structure |
| 40 | N-[(S)-1-(Hydroxymethyl)-2-(1H- indol-3-yl)ethyl]-3′,4′- dimethoxy[1,1′-biphenyl]-3- carboxamide; L-Tryptophanol and 3′,4′-Dimethoxy[1,1′-biphenyl]-3- carboxylic acid | 39 | (CDCl₃): 8.15 s (1H, NH); 7.87 s (1H, aryl); 7.73 d (J=8.0 Hz, 1H, aryl); 7.65 d (J=7.6 Hz, 1H, aryl); 7.51 d (J=7.6 Hz, 1H, aryl); 7.40 dd (J=8.0 Hz/8.0 Hz, 1H, aryl); 7.40 dd (J=7.6 Hz/7.6 Hz, 1H, | |
| # aryl); 7.22 dd (J=8.0 Hz/8.0 Hz, 1H, aryl); 7.15 d (J=8.0 Hz, 1H, aryl); 7.12 d (J=2.1 Hz, 1H, aryl); 7.09 dd (J=8.0 Hz/2.1 Hz, 1H, aryl); 7.07 s (1H, aryl); 6.94 d (J=8.0 Hz, 1H, aryl); 6.55 d (J=7.6 Hz, 1H, NH); 4.51 m (1H, CH); 3.94 s (6H, OCH₃); 3.85 dd (J=11.0 Hz/4.2 Hz, 1H, CH₂OH); 3.80 dd (J=11.0 Hz/5.4 Hz, 1H, CH₂OH); 3.18 d (J=6.7 Hz, 2H, CH₂). | | | | |
| # [α]_{D} = −45.7° (c = 0.980, MeOH/CH₂Cl₂ 1:1) | 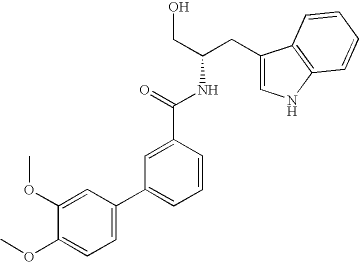 | | | |
| 41 | N-[(R)-1-(Hydroxymethyl)-2-(1H- indol-3-yl)ethyl]-3′,4′-dimethoxy- [1,1′-biphenyl]-3-carboxamide; D-Tryptophanol and 3′,4′-Dimethoxy[1,1′-biphenyl]-3- carboxylic acid | 39 | (CDCl₃): 8.14 s (1H, NH); 7.87 s (1H, aryl); 7.73 d (J=8.0 Hz, 1H, aryl); 7.65 d (J=7.6 Hz, 1H, aryl); 7.51 d (J=7.6 Hz, 1H, aryl); 7.40 dd (J=8.0 Hz/8.0 Hz, 1H, aryl); 7.40 dd (J=7.6 Hz/7.6 Hz, 1H, | |
| # aryl); 7.22 dd (J=8.0 Hz/8.0 Hz, 1H, aryl); 7.15 d (J=8.0 Hz, 1H, aryl); 7.13 d (J=2.1 Hz, 1H, aryl); 7.09 dd (J=8.0 Hz/2.1 Hz, 1H, aryl); 7.07 s (1H, aryl); 6.94 d (J=8.0 Hz, 1H, aryl); 6.55 d (J=7.6 Hz, 1H, NH); 4.51 m (1H, CH); 3.94 s (6H, OCH₃); 3.85 dd (J=11.0 Hz/4.2 Hz, 1H, CH₂OH); 3.80 dd (J=11.0 Hz/5.4 Hz, 1H, CH₂OH); 3.18 d (J=6.7 Hz, 2H, CH₂). | | | | |
| # [α]_{D} = +51.5° (c = 0.690, MeOH/CH₂Cl₂ 1:1) | 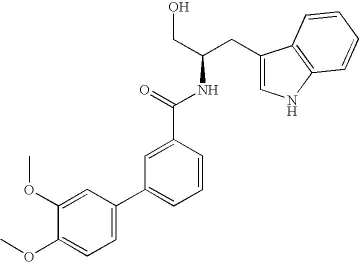 | | | |
| 42 | N-[(R)-1-(Hydroxymethyl)-2-(1H- indol-3-yl)ethyl]-2′,3′,4′- trimethoxy[1,1′-biphenyl]-3- carboxamide; D-Tryptophanol and 2′,3′,4′-Trimethoxy[1,1′- biphenyl]-3-carboxylic acid | 39 | (CDCl₃): 8.20 s (1H, NH); 7.77 s (1H, aryl); 7.71 d (J=7.8 Hz, 1H, aryl); 7.62 d (J=7.5 Hz, 1H, aryl); 7.60 d (J=8.0 Hz, 1H, aryl); 7.39 dd (J=8.0 Hz/7.5 Hz, 1H, aryl); 7.36 d (J=8.3 Hz, | |
| # 1H, aryl); 7.19 dd (J=7.8 Hz/7.0 Hz, 1H, aryl); 7.11 dd (J=8.3 Hz/7.0 Hz, 1H, aryl); 7.11 d (J=2.5 Hz, 1H, aryl); 6.97 d (J=8.6 Hz, 1H, aryl); 6.74 d (J=8.6 Hz, 1H, aryl); 6.55 d (J=7.3 Hz, 1H, NH); 4.49 m (1H, CH); 3.94 s (3H, OCH₃); 3.91 s (3H, OCH₃); 3.80 m (2H, CH₂OH); 3.62 s (3H, OCH₃); 3.16 d (J=6.8 Hz, 2H, CH₂). | 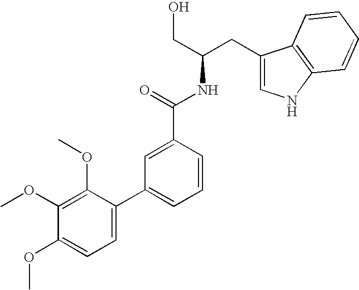 | | | |
| 43 | N-[(R)-1-(Hydroxymethyl)-2-(1H- indol-3-yl)ethyl]-3′,4′,5′-tri- methoxy[1,1′-biphenyl]-3- carboxamide; D-Tryptophanol and 3′,4′,5′-Trimethoxy[1,1′-bi- phenyl]-3-carboxylic acid | 39 | (CDCl₃): 8.16 s (1H, NH); 7.90 s (1H, aryl); 7.72 d (J=8.0 Hz, 1H, aryl); 7.65 d (J=7.8 Hz, 1H, aryl); 7.53 d (J=7.8 Hz, 1H, aryl); 7.41 dd (J=7.8 Hz/7.8 Hz, 1H, aryl); 7.38 d (J=8.0 | |
| # Hz, 1H, aryl); 7.21 dd (J=8.0 Hz/7.0 Hz, 1H, aryl); 7.12 dd (J=8.0 Hz/7.0 Hz, 1H, aryl); 7.12 d (J=2.3 Hz, 1H, aryl); 6.74 s (2H, aryl); 6.56 d (J=6.5 Hz, 1H, NH); 4.51 m (1H, CH); 3.91 s (6H, OCH₃); 3.90 s (3H, OCH₃); 3.82 m (2H, CH₂OH); 3.18 d (J=6.8 Hz, 2H, CH₂). | 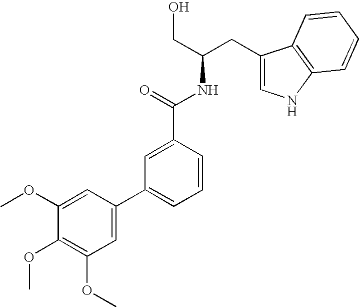 | | | |
| 44 | N-[(R)-1-(Hydroxymethyl)-2-(1H- indol-3-yl)ethyl]-3′,4′,5′-tri- methoxy[1,1′-biphenyl]-4- carboxamide; D-Tryptophanol and 3′,4′,5′-Trimethoxy[1,1′-bi- phenyl]-4-carboxylic acid | 39 | (CDCl₃): 8.18 s (1H, NH); 7.73 d (J=7.8 Hz, 1H, aryl); 7.70 d (J=8.5 Hz, 2H, aryl); 7.54 d (J=8.5 Hz, 2H, aryl); 7.39 d (J=8.0 Hz, 1H, aryl); 7.23 dd (J=7.8 Hz/7.0 Hz, 1H, aryl); 7.17 dd | |
| # (J=8.0 Hz/7.0 Hz, 1H, aryl); 7.12 d (J=2.3 Hz, 1H, aryl); 6.76 s (2H, aryl); 6.54 d (J=7.5 Hz, 1H, NH); 4.51 m (1H, CH); 3.92 s (6H, OCH₃); 3.89 s (3H, OCH₃); 3.82 m (2H, CH₂OH); 3.18 d (J=6.8 Hz, 2H, CH₂). | 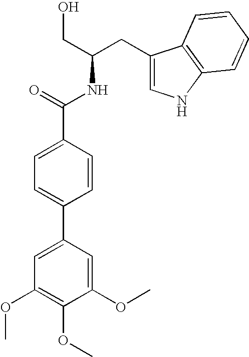 | | | |
| 45 | N-[(R)-1-(Hydroxymethyl)-2-(1H- indol-3-yl)ethyl]-3′,4′,5′-tri- methoxy-2-methyl[1,1′-biphenyl]- 4-carboxamide; D-Tryptophanol and 3′,4′,5′-Trimethoxy-2- methyl[1,1′-biphenyl]-4- carboxylic acid | 39 | CDCl₃): 8.24 s (1H, NH); 7.74 d (J=8.0 Hz, 1H, aryl); 7.52 m (1H, aryl); 7.48 d (J=8.0 Hz, 1H, aryl); 7.39 d (J=8.0 Hz, 1H, aryl); 7.23 d (J=8.0 Hz, 1H, aryl); 7.23 dd | |
| # (J=8.0 Hz/7.0 Hz, 1H, aryl); 7.16 dd (J=8.0 Hz/7.0 Hz, 1H, aryl); 7.12 d (J=2.3 Hz, 1H, aryl); 6.46 s (2H, aryl); 6.53 d (J=7.3 Hz, 1H, NH); 4.50 m (1H, CH); 3.90 s (3H, OCH₃); 3.85 s (6H, OCH₃); 3.82 m (2H, CH₂OH); 3.18 d (J=6.8 Hz, 2H, CH₂). | 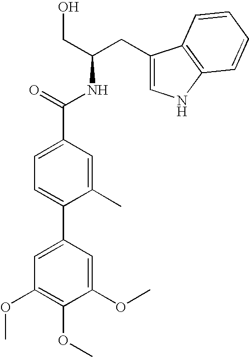 | | | |
| 46 | N-[(R)-1-(Hydroxymethyl)-2-(1H- indol-3-yl)ethyl]-2′,3′,4′-tri- methoxy[1,1′-biphenyl]-2- carboxamide; D-Tryptophanol and 2′,3′,4′-Trimethoxy[1,1′- biphenyl]-2-carboxylic acid | 39 | (CDCl₃): 8.09 s (1H, NH); 7.66 d (J=7.5 Hz, 1H, aryl); 7.56 d (J=8.0 Hz, 1H, aryl); 7.44 dd (J=7.5 Hz/7.5 Hz, 1H, aryl); 7.38 dd (J=7.5 Hz/7.5 Hz, 1H, aryl); 7.33 d (J=8.0 Hz, 1H, aryl); | |
| # 7.24 d (J=7.5 Hz, 1H, aryl); 7.18 dd (J=8.0 Hz/7.0 Hz, 1H, aryl); 7.08 dd (J=8.0 Hz/7.0 Hz, 1H, aryl); 6.92 d (J=1.5 Hz, 1H, aryl); 6.86 d (J=8.5 Hz, 1H, aryl); 6.65 d (J=8.5 Hz, 1H, aryl); 6.04 d (J=8.0 Hz, 1H, NH); 4.26 m (1H, CH); 3.90 s (3H, OCH₃); 3.85 s (3H, OCH₃); 3.59 m (1H, CH₂OH); 3.57 s (3H, OCH₃); 3.46 m (1H, CH₂OH); 2.84 m (2H, CH₂). | 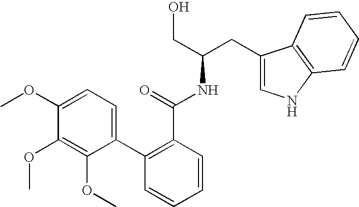 | | | |
| 47 | N-[(R)-1-(Hydroxymethyl)-2-(1H- indol-3-yl)ethyl]-3′,4′,5′-tri- methoxy[1,1′-biphenyl]-2- carboxamide; D-Tryptophanol and 3′,4′,5′-Trimethoxy[1,1′-bi- phenyl]-2-carboxylic acid | 39 | (CDCl₃): 8.09 s (1H, NH); 7.65 d (J=7.5 Hz, 1H, aryl); 7.54 d (J=8.0 Hz, 1H, aryl); 7.46 dd (J=7.5 Hz/7.5 Hz, 1H, aryl); 7.40 dd (J=7.5 Hz/7.5 Hz, 1H, aryl); 7.34 d (J=7.5 Hz, 1H, aryl); | |
| # 7.33 d (J=8.0 Hz, 1H, aryl); 7.17 dd (J=8.0 Hz/7.0 Hz, 1H, aryl); 7.07 dd (J=8.0 Hz/7.0 Hz, 1H, aryl); 6.75 d (J=2.5 Hz, 1H, aryl); 6.59 s (2H, aryl); 5.68 d (J=7.3 Hz, 1H, NH); 4.25 m (1H, CH); 3.87 s (3H, OCH₃); 3.83 s (6H, OCH₃); 3.50 m (2H, CH₂OH); 2.78 d (J=7.0 Hz, 2H, CH₂). | 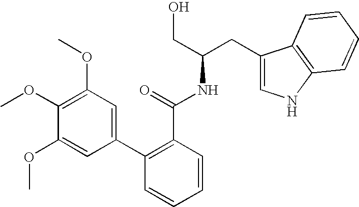 | | | |
| 48 | N-[(R)-1-(Hydroxymethyl)-2-(1H- indol-3-yl)ethyl]-2′,3′,4′-tri- methoxy[1,1′-biphenyl]- 3-carboxamide; D-Tryptophanol and 2′,3′,4′-Trimethoxy-6- methyl[1,1′-biphenyl]-3- carboxylic acid | 39 | (CDCl₃): 8.16 s (1H, NH); 7.69 d (J=8.0 Hz, 1H, aryl); 7.55 dd (J=8.0 Hz/2.0 Hz, 1H, aryl); 7.45 d (J=2.0 Hz, 1H, aryl); 7.34 d (J=8.0 Hz, 1H, aryl); 7.25 d (J=8.0 Hz, 1H, | |
| # aryl); 7.17 dd (J=8.0 Hz/7.0 Hz, 1H, aryl); 7.08 dd (J=8.0 Hz/7.0 Hz, 1H, aryl); 7.09 d (J=2.3 Hz, 1H, aryl); 6.78 d (J=8.5 Hz, 1H, aryl); 6.72 d (J=8.5 Hz, 1H, aryl); 6.48d (J=7.0 Hz, 1H, NH); 4.45 m (1H, CH); 3.93 s (3H, OCH₃); 3.92 s (3H, OCH₃); 3.79 m (2H, CH₂OH); 3.52 s (3H, OCH₃); 3.14 m (2H, CH₂); 2.19 s (3H, CH₃). | 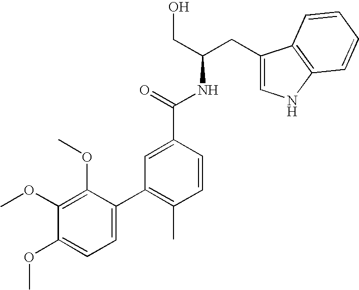 | | | |
| 49 | N-[(R)-1-(Hydroxymethyl)-2-(1H- indol-3-yl)ethyl]-3′,4′,5′-tri- methoxy-6-methyl[1,1′-biphenyl]- 3-carboxamide; D-Tryptophanol and 3′,4′,5′-Trimethoxy-6- methyl[1,1′-biphenyl]-3- carboxylic acid | 39 | (CDCl₃): 8.15 s (1H, NH); 7.69 d (J=7.8 Hz, 1H, aryl); 7.53 dd (J=8.0 Hz/2.0 Hz, 1H, aryl); 7.50 d (J=2.0 Hz, 1H, aryl); 7.35 d (J=8.0 Hz, 1H, aryl); 7.26 d (J=8.0 Hz, | |
| # 1H, aryl); 7.18 dd (J=8.0 Hz/7.0 Hz, 1H, aryl); 7.07 dd (J=8.0 Hz/7.0 Hz, 1H, aryl); 7.10 d (J=2.3 Hz, 1H, aryl); 6.44 s (2H, aryl); 6.49 d (J=6.8 Hz, 1H, NH); 4.48 m (1H, CH); 3.92 s (3H, OCH₃); 3.85 s (6H, OCH₃); 3.79 m (2H, CH₂OH); 3.15 m (2H, CH₂); 2.29 s (3H, CH₃). | 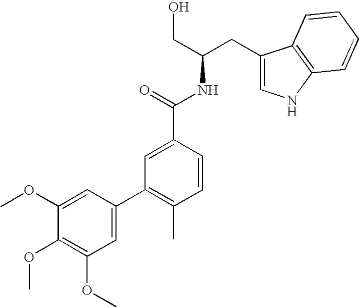 | | | |
| 50 | N-[(R)-1-(Hydroxymethyl)-2-(1H- indol-3-yl)ethyl]-2′,3′,4′-tri- methoxy[1,1′-biphenyl]-4- carboxamide; D-Tryptophanol und 2′,3′,4′-Trimethoxy[1,1′-bi- phenyl]-4-carboxylic acid | 39 | (CDCl₃): 8.21 s (1H, NH); 7.73 d (J=8.0 Hz, 1H, aryl); 7.68 d (J=8.5 Hz, 2H, aryl); 7.51 d (J=8.5 Hz, 2H, aryl); 7.38 d (J=8.0 Hz, 1H, aryl); 7.22 dd (J=7.8 Hz/7.0 Hz, 1H, aryl); 7.15 dd | |
| # (J=8.0 Hz/7.0 Hz, 1H, aryl); 7.11 d (J=2.3 Hz, 1H, aryl); 7.01 d (J=8.6 Hz, 1H, aryl); 6.74 d (J=8.6 Hz, 1H, aryl); 6.55 d (J=7.2 Hz, 1H, NH); 4.51 m (1H, CH); 3.93 s (3H, OCH₃); 3.90 s (3H, OCH₃); 3.81 m (2H, CH₂OH); 3.64 s (3H, OCH₃); 3.17 d (J=6.8 Hz, 2H, CH₂). | 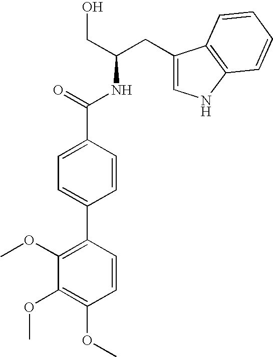 | | | |
| 51 | N-[(R)-1-(Hydroxymethyl)-2-(1H- indol-3-yl)ethyl]-2′,3′,4′- trimethoxy-2-methyl[1,1′- biphenyl]-4-carboxamide; D-Tryptophanol and 2′,3′,4′-Trimethoxy-2-methyl- [1,1′-biphenyl]-4-carboxylic acid | 39 | (CDCl₃): 8.19 s (1H, NH); 7.74 d (J=8.0 Hz, 1H, aryl); 7.50 s (1H, aryl); 7.47 d (J=8.0 Hz, 1H, aryl); 7.39 d (J=8.0 Hz, 1H, aryl); 7.22 dd (J=8.0 Hz/7.0 Hz, 1H, aryl); | |
| # 7.19 d (J=8.0 Hz, 1H, aryl); 7.16 dd (J=8.0 Hz/7.0 Hz, 1H, aryl); 7.12 d (J=2.3 Hz, 1H, aryl); 6.78 d (J=8.6 Hz, 1H, aryl); 6.71 d (J=8.6 Hz, 1H, aryl); 6.51 d (J=7.0 Hz, 1H, NH); 4.50 m (1H, CH); 3.92 s (3H, OCH₃); 3.90 s (3H, OCH₃); 3.82 m (2H, CH₂OH); 3.54 s (3H, OCH₃); 3.18 m (2H, CH₂); 2.14 s (3H, CH₃). | 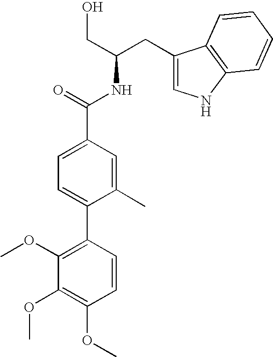 | | | |
| 52 | N-[(R)-1-(Hydroxymethyl)-2-(1H- indol-3-yl)ethyl]-2′,3′,4,4′-tetra- methoxy[1,1′-biphenyl]-2- carboxamide; D-Tryptophanol and 2′,3′,4,4′-Tetramethoxy[1,1′- biphenyl]-4-carboxylic acid | 39 | (CDCl₃): 8.07 s (1H, NH); 7.56 d (J=8.0 Hz, 1H, aryl); 7.33 d (J=8.0 Hz, 1H, aryl); 7.22 d (J=2.8 Hz, 1H, aryl); 7.17 dd (J=7.8 Hz/7.0 Hz, 1H, aryl); 7.14 d (J=8.5 Hz, 1H, aryl); | |
| # 7.08 dd (J=8.0 Hz/7.0 Hz, 1H, aryl); 6.98 dd (J=8.5 Hz/2.8 Hz, 1H, aryl); 6.98 d (J=2.3 Hz, 1H, aryl); 6.83 d (J=8.7 Hz, 1H, aryl); 6.63 d (J=8.7 Hz, 1H, aryl); 6.05 d (J=7.9 Hz, 1H, NH); 4.25 m (1H, CH); 3.90 s (3H, OCH₃); 3.83 s (6H, OCH₃); 3.58 s (3H, OCH₃); 3.58 m (1H, CH₂OH); 3.44 m (1H, CH₂OH); 2.81 m (2H, CH₂). | 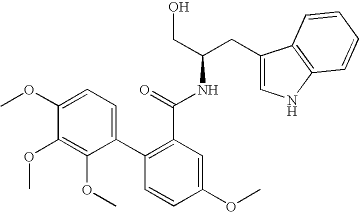 | | | |
| 53 | N-[(R)-1-(Hydroxymethyl)-2-(1H- indol-3-yl)ethyl]-3′,4,4′,5′- tetramethoxy[1,1′-biphenyl]-2- carboxamide; D-Tryptophanol and 3′,4,4′,5′-Tetramethoxy[1,1′- biphenyl]-4-carboxylic acid | 39 | (DMSO-d₆): 10.79 s (1H, NH); 7.81 d (J=8.1 Hz, 1H, NH); 7.58 d (J=7.9 Hz, 1H, aryl); 7.33 d (J=8.5 Hz, 1H, aryl); 7.32 d (J=7.6 Hz, 1H, aryl); 7.09 d (J=2.8 Hz, 1H, aryl); 7.05 dd | |
| # (J=7.6 Hz/7.0 Hz, 1H, aryl); 7.02 dd (J=8.5 Hz/2.8 Hz, 1H, aryl); 6.96 dd (J=7.9 Hz/7.0 Hz, 1H, aryl); 6.80 d (J=2.6 Hz, 1H, aryl); 6.62 s (2H, aryl); 4.01 m (1H, CH); 3.76 s (3H, OCH₃); 3.73 s (6H, OCH₃); 3.64 s (3H, OCH₃); 3.35 m (1H, CH₂OH); 3.25 m (1H, CH₂OH); 2.83 dd (J=14.3 Hz/7.0 Hz, 1H, CH₂); 2.71 dd (J=14.3 Hz/7.0 Hz, 1H, CH₂). | 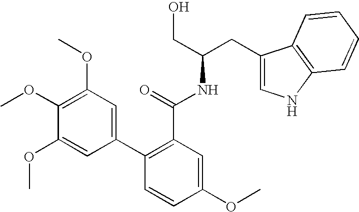 | | | |
| 54 | 4′-(Hydroxymethyl)-N-[(R)-1- (hydroxymethyl)-2-(1H-indol-3- yl)ethyl]-6-methyl[1,1′-biphenyl]- 3-carboxamide; D-Tryptophanol and 4′-(Hydroxymethyl)-6-methyl- [1,1′-biphenyl]-3-carboxylic acid | 39 | (CD₃OD): 7.66 d (J=7.8 Hz, 1H, aryl); 7.64 dd (J=7.5 Hz/2.0 Hz, 1H, aryl); 7.58 d (J=2.0 Hz, 1H, aryl); 7.43 d (J=8.5 Hz, 2H, aryl); 7.32 d (J=8.0 Hz, 1H, aryl); 7.31 d (J=7.5 Hz, | |
| # 1H, aryl); 7.29 d (J=8.5 Hz, 2H, aryl); 7.10 s (1H, aryl); 7.06 dd (J=7.8 Hz/7.0 Hz, 1H, aryl); 6.96 dd (J=8.0 Hz/7.0 Hz, 1H, aryl); 4.67 s (2H, CH₂OH); 4.44 m (1H, CH); 3.68 m (2H, CH₂OH); 3.13 dd (J=14.6 Hz/6.8 Hz, 1H, CH₂); 3.05 dd (J=14.6 Hz/8.0 Hz, 1H, CH₂); 2.27 s (3H, CH₃). | 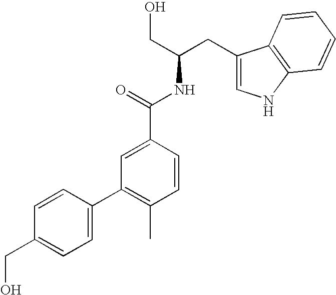 | | | |
| 55 | 4′-(Hydroxymethyl)-N-[(R)-1- (hydroxymethyl)-2-(1H-indol-3- yl)ethyl]-2-methyl[1,1′-biphenyl]- 4-carboxamide; D-Tryptophanol and 4′-(Hydroxymethyl)-2- methyl[1,1′-biphenyl]-4- carboxylic acid | 39 | (CD₃OD): 7.69 d (J=7.8 Hz, 1H, aryl); 7.63 s (1H, aryl); 7.61 d (J=7.8 Hz, 1H, aryl); 7.43 d (J=8.0 Hz, 2H, aryl); 7.32 d (J=8.3 Hz, 1H, aryl); 7.28 d (J=8.0 Hz, 2H, aryl); 7.23 | |
| # d (J=7.8 Hz, 1H, aryl); 7.12 s (1H, aryl); 7.08 dd (J=7.8 Hz/7.0 Hz, 1H, aryl); 7.01 dd (J=8.3 Hz/7.0 Hz, 1H, aryl); 4.66 s (2H, CH₂OH); 4.46 m (1H, CH); 3.71 m (2H, CH₂OH); 3.16 dd (J=14.6 Hz/6.8 Hz, 1H, CH₂); 3.07 dd (J=14.6 Hz/7.0 Hz, 1H, CH₂); 2.26 s (3H, CH₃). | 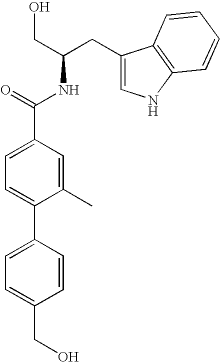 | | | |
| 56 | 4′-(Hydroxymethyl)-N-[(R)-1- (hydroxymethyl)-2-(1H-indol-3- yl)ethyl][1,1′-biphenyl]-2- carboxamide; D-Tryptophanol and 4′-(Hydroxymethyl)[1,1′-bi- phenyl]-2-carboxylic acid | 39 | (CD₃OD): 7.57 d (J=7.8 Hz, 1H, aryl); 7.46 dd (J=7.3 Hz/7.3 Hz, 1H, aryl); 7.38 dd (J=7.3 Hz/7.3 Hz, 1H, aryl); 7.37 d (J=7.8 Hz, 1H, aryl); 7.35 d (J=7.3 Hz, 2H, aryl); 7.29 d (J=8.0 Hz, 2H, aryl); | |
| # 7.24 d (J=8.0 Hz, 2H, aryl); 7.10 dd (J=7.8 Hz/7.0 Hz, 1H, aryl); 6.99 dd (J=7.8 Hz/7.0 Hz, 1H, aryl); 6.97 s (1H, aryl); 4.56 s (2H, CH₂OH); 4.22 m (1H, CH); 3.49 dd (J=11.0 Hz/5.2 Hz, 1H, CH₂OH); 3.41 dd (J=11.0 Hz/5.7 Hz, 1H, CH₂OH); 2.93 dd (J=14.6 Hz/6.5 Hz, 1H, CH₂); 2.81 dd (J=14.6 Hz/7.3 Hz, 1H, CH₂). | 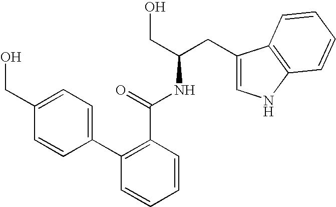 | | | |
| 57 | 4′-(Hydroxymethyl)-N-[(R)-1- (hydroxymethyl)-2-(1H-indol-3- yl)ethyl][1,1′-biphenyl]-3- carboxamide; D-Tryptophanol and 4′-(Hydroxymethyl)[1,1′-bi- phenyl]-3-carboxylic acid | 39 | (CD₃OD): 7.95 d (J=1.8 Hz/1.5 Hz, 1H, aryl); 7.75 d (J=7.8 Hz, 1H, aryl); 7.71 d(J=7.8 Hz, 1H, aryl); 7.68 d (J=7.8 Hz, 1H, aryl); 7.61 d (J=8.0 Hz, 2H, aryl); 7.48 dd (J=7.8 Hz/7.8 Hz, 1H, | |
| # aryl); 7.45 d (J=8.0 Hz, 2H, aryl); 7.32 d (J=8.0 Hz, 1H, aryl); 7.13 s (1H, aryl); 7.08 dd (J=7.8 Hz/7.0 Hz, 1H, aryl); 6.99 dd (J=8.0 Hz/7.0 Hz, 1H, aryl); 4.66 s (2H, CH₂OH); 4.47 m (1H, CH); 3.72 m (2H, CH₂OH); 3.17 dd (J=14.8 Hz/6.8 Hz, 1H, CH₂); 3.08 dd (J=14.8 Hz/7.9 Hz, 1H, CH₂). | 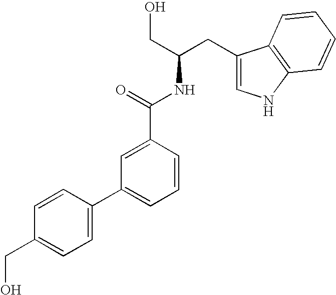 | | | |
| 58 | N-[(R)-1-(Hydroxymethyl)-2-(1H- indol-3-yl)ethyl]-4-methoxy-3′-(1- methylethyl)[1,1′-biphenyl]-2- carboxamide D-Tryptophanol 4-Methoxy-3′-(1-methylethyl)- [1,1′-biphenyl]-2-carboxylic acid | 39 | (CDCl₃): 7.97 s (1H, NH); 7.54 d (J=8.0 Hz, 1H, aryl); 7.33 d (J=8.0 Hz, 1H, aryl); 7.32 dd (J=8.2 Hz/7.6 Hz, 1H, aryl); 7.26 d (J=8.5 Hz, 1H, aryl); 7.25 d (J=8.2 Hz, 1H, aryl); 7.22 s (1H, | |
| # aryl); 7.18 dd (J=8.0 Hz/7.0 Hz, 1H, aryl); 7.16 d (J=7.6 Hz, 1H, aryl); 7.08 dd (J=8.0 Hz/7.0 Hz, 1H, aryl); 7.01 dd (J=8.5 Hz/2.8 Hz, 1H, aryl); 6.98 d (J=2.3 Hz, 1H, aryl); 6.82 d (J=2.8 Hz, 1H, aryl); 5.54 d (J=7.5 Hz, 1H, NH); 4.20 m (1H, CH); 3.85 s (3H, OCH₃); 3.38 m (2H, CH₂OH); 2.93 sept (J=7.0 Hz, 1H, CH); 2.70 m (2H, CH₂); 1.26 d (J=7.0 Hz, 6H, CH₃). | 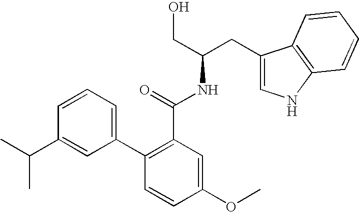 | | | |
| 59 | N-[(R)-1-(Hydroxymethyl)-2-(1H- indol-3-yl)ethyl]-3′-(1-methyl- ethyl)[1,1′-biphenyl]-3- carboxamide D-Tryptophanol and 3′-(1-Methylethyl)[1,1′-biphenyl]- 3-carboxylic acid | 39 | (CDCl₃): 8.12 s (1H, NH); 7.88 dd (J=1.8 Hz/1.5 Hz, 1H, aryl); 7.73 d (J=8.0 Hz, 1H, aryl); 7.69 d (J=7.5 Hz, 1H, aryl); 7.57 d (J=7.3 Hz, 1H, aryl); 7.42 dd (J=7.5 Hz/7.3 Hz, 1H, aryl); 7.41 s (1H, | |
| # aryl); 7.38 d (J=7.3 Hz, 1H, aryl); 7.37 dd (J=7.3 Hz/7.3 Hz, 1H, aryl); 7.33 d (J=7.3 Hz, 1H, aryl); 7.25 d (J=8.0 Hz, 1H, aryl); 7.22 dd (J=8.0 Hz/7.0 Hz, 1H, aryl); 7.14 dd (J=8.0 Hz/7.0 Hz, 1H, aryl); 7.12 d (J=1.8 Hz, 1H, aryl); 6.54 d (J=7.0 Hz, 1H, NH); 4.51 m (1H, CH); 3.82 m (2H, CH₂OH); 2.98 sept (J=6.8 Hz, 1H, CH); 3.18 m (2H, CH₂); 1.30 d (J=6.8 Hz, 6H, | | | | |
| # CH₃). | 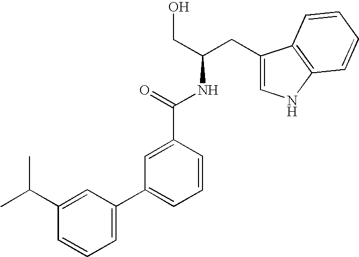 | | | |
| 60 | N-[(R)-1-(Hydroxymethyl)-2-(1H- indol-3-yl)ethyl]-6-methyl-3′-(1- methylethyl)[1,1′-biphenyl]-3- carboxamide D-Tryptophanol and 6-Methyl-3′-(1-methylethyl)[1,1′- biphenyl]-3-carboxylic acid | 39 | (CDCl₃): 8.12 s (1H, NH); 7.70 d (J=8.0 Hz, 1H, aryl); 7.55 dd (J=7.7 Hz/2.0 Hz, 1H, aryl); 7.50 d (J=2.0 Hz, 1H, aryl); 7.34 dd (J=8.0 Hz/7.8 Hz, 1H, aryl); 7.34 d (J=8.0 Hz, 1H, aryl); | |
| # 7.27 d (J=8.0 Hz, 1H, aryl); 7.23 d (J=7.8 Hz, 1H, aryl); 7.17 dd (J=8.0 Hz/7.0 Hz, 1H, aryl); 7.12 s (1H, aryl); 7.09 dd (J=8.0 Hz/7.0 Hz, 1H, aryl); 7.09 d (J=1.8 Hz, 1H, aryl); 7.06 d (J=7.7 Hz, 1H, aryl); 6.48 d (J=7.0 Hz, 1H, NH); 4.47 m (1H, CH); 3.82 dd (J=11.0 Hz/3.5 Hz, 1H, CH₂OH); 3.76 dd (J=11.0 Hz/5.3 Hz, 1H, CH₂OH); 3.15 m (2H, CH₂); 2.95 sept (J=7.0 Hz, 1H, | | | | |
| # CH); 2.27 s (3H, CH₃); 1.29 d (J=7.0 Hz, 6H, CH₃). | 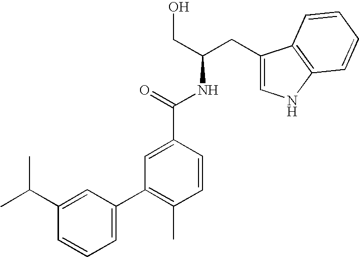 | | | |
| 61 | N-[(R)-1-(Hydroxymethyl)-2-(1H- indol-3-yl)ethyl]-3′-(1-methyl- ethyl)[1,1′-biphenyl]-4- carboxamide; D-Tryptophanol and 3′-(1-Methylethyl)[1,1′-biphenyl]- 4-carboxylic acid | 39 | (CDCl₃): 8.14 s (1H, NH); 7.74 d (J=8.0 Hz, 1H, aryl); 7.71 d (J=8.5 Hz, 2H, aryl); 7.59 d (J=8.5 Hz, 2H, aryl); 7.43 s (1H, aryl); 7.40 d (J=8.0 Hz, 1H, aryl); 7.39 d (J=8.0 Hz, 1H, aryl); 7.37 dd | |
| # (J=8.0 Hz/8.0 Hz, 1H, aryl); 7.26 m (1H, aryl); 7.23 dd (J=8.0 Hz/7.0 Hz, 1H, aryl); 7.16 dd (J=8.0 Hz/7.0 Hz, 1H, aryl); 7.13 d (J=2.5 Hz, 1H, aryl); 6.53 d (J=7.0 Hz, 1H, NH); 4.51 m (1H, CH); 3.83 m (2H, CH₂OH); 3.19 m (2H, CH₂); 2.98 sept (J=7.0 Hz, 1H, CH); 1.30 d (J=7.0 Hz, 6H, CH₃). | 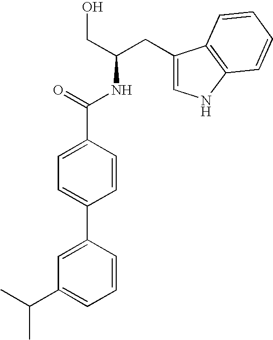 | | | |
| 62 | N-[(R)-1-(Hydroxymethyl)-2-(1H- indol-3-yl)ethyl]-2-methyl-3′-(1- methylethyl)[1,1′-biphenyl]-4- carboxamide; D-Tryptophanol and 2-Methyl-3′-(1-methylethyl)[1,1′- biphenyl]-4-carboxylic acid | 39 | (CDCl₃): 8.16 s (1H, NH); 7.74 d (J=8.0 Hz, 1H, aryl); 7.47 d (J=8.0 Hz, 1H, aryl); 7.09 d (J=8.0 Hz, 1H, aryl); 7.13 s (1H, aryl); 7.39 d (J=8.0 Hz, 1H, aryl); 7.23 m (2H, aryl); 7.34 dd | |
| # (J=8.0 Hz/8.0 Hz, 1H, aryl); 7.52 s (1H, aryl); 7.23 dd (J=8.0 Hz/7.0 Hz, 1H, aryl); 7.17 dd (J=8.0 Hz/7.0 Hz, 1H, aryl); 7.13 d (J=1.5 Hz, 1H, aryl); 6.51 d (J=6.8 Hz, 1H, NH); 4.50 m (1H, CH); 3.83 m (2H, CH₂OH); 3.18 m (2H, CH₂); 2.94 sept (J=7.0 Hz, 1H, CH); 2.24 s (3H, CH₃); 1.28 d (J=7.0 Hz, 6H, CH₃). | 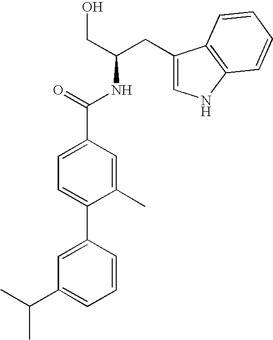 | | | |
| 63 | 4′-(Hydroxymethyl)-N-[(R)-1- (hydroxymethyl)-2-(1H-indol-3- yl)ethyl]-4-methoxy[1,1′-bi- phenyl]-2-carboxamide; D-Tryptophanol and 4′-(Hydroxymethyl)-4-methoxy- [1,1′-biphenyl]-2-carboxylic acid | 39 | (CD₃OD): 7.58 d (J=8.0 Hz, 1H, aryl); 7.34 d (J=8.0 Hz, 1H, aryl); 7.27 d (J=8.3 Hz, 1H, aryl); 7.25 d (J=8.3 Hz, 2H, aryl); 7.21 d (J=8.3 Hz, 2H, aryl); 7.09 dd (J=7.8 Hz/7.0 Hz, | |
| # 1H, aryl); 7.02 d (J=8.3 Hz, 1H, aryl); 7.00 dd (J=7.8 Hz/7.0 Hz, 1H, aryl); 6.98 s (1H, aryl); 6.89 d (J=2.8 Hz, 1H, aryl); 4.55 s (2H, CH₂OH); 4.22 m (1H, CH); 3.79 s (3H, OCH₃); 3.50 dd (J=10.8 Hz/5.1 Hz, 1H, CH₂OH); 3.43 dd (J=10.8 Hz/5.6 Hz, 1H, CH₂OH); 2.94 dd (J=14.7 Hz/6.3 Hz, 1H, CH₂); 2.82 dd (J=14.7 Hz/7.6 Hz, 1H, CH₂). | 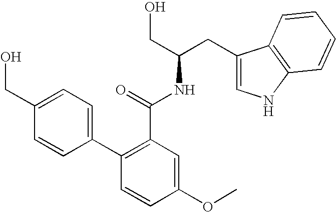 | | | |
| 64 | 3′,4′,5′-Trifluoro-N-[(R)-1- (hydroxymethyl)-2-(1H-indol-3- yl)ethyl][1,1′-biphenyl]-2- carboxamide; D-Tryptophanol and 3′,4′,5′-Trifluoro[1,1′-biphenyl]-2- carboxylic acid | 39 | (CD₃OD): 7.59 d (J=8.0 Hz, 1H, aryl); 7.49 dd (J=7.3 Hz/7.3 Hz, 1H, aryl); 7.39 dd (J=7.3 Hz/7.3 Hz, 1H, aryl); 7.34d (J=8.0 Hz, 1H, aryl); 7.34 d (J=7.3 Hz, 1H, aryl); 7.32 d (J=7.3 Hz, | |
| # 1H, aryl); 7.11 m (2H, aryl); 7.11 dd (J=8.0 Hz/7.0 Hz, 1H, aryl); 7.06 d (J=1.8 Hz, 1H, aryl); 6.99 dd (J=8.0 Hz/7.0 Hz, 1H, aryl); 4.27 m (1H, CH); 3.53 m (2H, CH₂OH); 3.00 dd (J=15.1 Hz/6.8 Hz, 1H, CH₂); 2.87 dd (J=15.1 Hz/6.8 Hz, 1H, CH₂). | 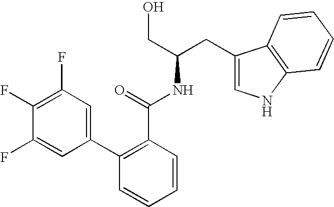 | | | |
| 65 | 3′,4′,5′-Trifluoro-N-[(R)-1- (hydroxymethyl)-2-(1H-indol-3- yl)ethyl][1,1′-biphenyl]-3- carboxamide; D-Tryptophanol and 3′,4′,5′-Trifluoro[1,1′-biphenyl]-3- carboxylic acid | 39 | (CD₃OD): 7.88 s (1H, aryl); 7.78 d (J=7.7 Hz, 1H, aryl); 7.75 d (J=7.7 Hz, 1H, aryl); 7.66 d (J=8.0 Hz, 1H, aryl); 7.51 dd (J=7.7 Hz/7.7 Hz, 1H, aryl); 7.42 m (2H, aryl); 7.32 d (J=8.0 Hz, 1H, | |
| # aryl); 7.12 d (J=1.8 Hz, 1H, aryl); 7.06 dd (J=8.0 Hz/7.0 Hz, 1H, aryl); 6.97 dd (J=8.0 Hz/7.0 Hz, 1H, aryl); 4.48 m (1H, CH); 3.72 m (2H, CH₂OH); 3.16 dd (J=14.5 Hz/6.4 Hz, 1H, CH₂); 3.07 dd (J=14.5 Hz/7.2 Hz, 1H, CH₂). | 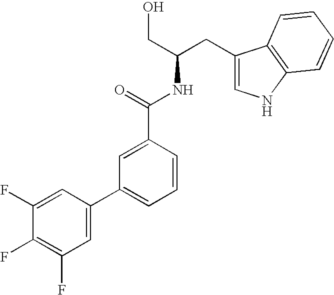 | | | |
| 66 | 3′,4′,5′-Trifluoro-N-[(R)-1- (hydroxymethyl)-2-(1H-indol-3- yl)ethyl]-6-methyl[1,1′-biphenyl]- 3-carboxamide; D-Tryptophanol and 3′,4′,5′-Trifluoro-6-methyl[1,1′- biphenyl]-3-carboxylic acid | 39 | (CD₃OD): 7.68 dd (J=7.9 Hz/1.9 Hz, 1H, aryl); 7.64 d (J=8.0 Hz, 1H, aryl); 7.50 d (J=1.9 Hz, 1H, aryl); 7.35 d (J=7.9 Hz, 1H, aryl); 7.30 d (J=8.0 Hz, 1H, aryl); 7.10 m | |
| # (2H, aryl); 7.10 d (J=1.8 Hz, 1H, aryl); 7.04 dd (J=8.0 Hz/7.0 Hz, 1H, aryl); 6.94 dd (J=8.0 Hz/7.0 Hz, 1H, aryl); 4.44 m (1H, CH); 3.69 m (2H, CH₂OH); 3.13 dd (J=14.5 Hz/6.7 Hz, 1H, CH₂); 3.04 dd (J=14.5 Hz/7.2 Hz, 1H, CH₂); 2.29 s (3H, CH₃). | 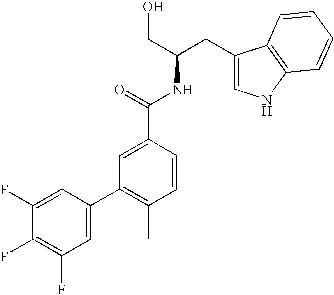 | | | |
| 67 | 3′,4′,5′-Trifluoro-N-[(R)-1- (hydroxymethyl)-2-(1H-indol-3- yl)ethyl][1,1′-biphenyl]-4- carboxamide; D-Tryptophanol and 3′,4′,5′-Trifluoro[1,1′-biphenyl]-4- carboxylic acid | 39 | (CD₃OD): 7.84 d (J=8.7 Hz, 2H, aryl); 7.67 d (J=8.7 Hz, 2H, aryl); 7.67 d (J=8.0 Hz, 1H, aryl); 7.46 m (2H, aryl); 7.32 d (J=8.0 Hz, 1H, aryl); 7.11 d (J=1.8 Hz, 1H, aryl); 7.08 dd (J=8.0 | |
| # Hz/7.0 Hz, 1H, aryl); 7.00 dd (J=8.0 Hz/7.0 Hz, 1H, aryl); 4.47 m (1H, CH); 3.71 m (2H, CH₂OH); 3.16 dd (J=15.1 Hz/6.8 Hz, 1H, CH₂); 3.06 dd (J=15.1 Hz/7.3 Hz, 1H, CH₂). | 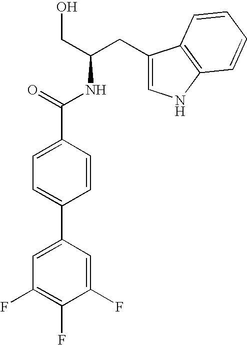 | | | |
| 68 | 3′,4′,5′-Trifluoro-N-[(R)-1- (hydroxymethyl)-2-(1H-indol-3- yl)ethyl]-2-methyl[1,1′-biphenyl]- 4-carboxamide; D-Tryptophanol and 3′,4′,5′-Trifluoro-2-methyl[1,1′- biphenyl]-4-carboxylic acid | 39 | (CD₃OD): 7.68 d (J=7.7 Hz, 1H, aryl); 7.64 s (1H, aryl); 7.63 d (J=8.0 Hz, 1H, aryl); 7.32 d (J=8.0 Hz, 1H, aryl); 7.25 d (J=7.7 Hz, 1H, aryl); 7.11 m (2H, aryl); 7.08 dd | |
| # (J=8.0 Hz/7.0 Hz, 1H, aryl); 7.08 d (J=1.8 Hz, 1H, aryl); 7.00 dd (J=8.0 Hz/7.0 Hz, 1H, aryl); 4.46 m (1H, CH); 3.71 m (2H, CH₂OH); 3.16 dd (J=14.8 Hz/7.2 Hz, 1H, CH₂); 3.07 dd (J=14.8 Hz/7.3 Hz, 1H, CH₂); 2.28 s (3H, CH₃). | 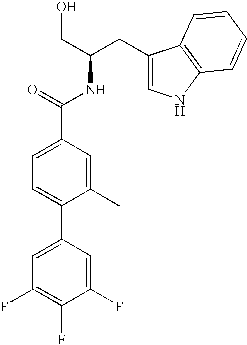 | | | |
| 69 | N-[(R)-1-(Hydroxymethyl)-2-(1H- indol-3-yl)ethyl]-2′,5′-dimethoxy- [1,1′-biphenyl]-2-carboxamide; D-Tryptophanol und 2′,5′-Dimethoxy[1,1′-biphenyl]-2- carboxylic acid | 39 | (CD₃OD): 7.57 d (J=8.0 Hz, 1H, aryl); 7.51 d (J=7.5 Hz, 1H, aryl); 7.47 dd (J=7.5 Hz/7.5 Hz, 1H, aryl); 7.38 dd (J=7.5 Hz/7.5 Hz, 1H, aryl); 7.31 d (J=8.0 Hz, 1H, aryl); 7.26 d (J=7.5 Hz, 1H, aryl); | |
| # 7.07 dd (J=8.0 Hz/7.0 Hz, 1H, aryl); 6.98 d (J=1.8 Hz, 1H, aryl); 6.97 dd (J=8.0 Hz/7.0 Hz, 1H, aryl); 6.86 m (2H, aryl); 6.77 s (1H, aryl); 4.16 m (1H, CH); 3.73 s (3H, OCH₃); 3.60 s (3H, OCH₃); 3.39 dd (J=10.9 Hz/4.5 Hz, 1H, CH₂OH); 3.30 dd (J=10.9 Hz/5.3 Hz, 1H, CH₂OH); 2.80 dd (J=14.7 Hz/7.6 Hz, 1H, CH₂); 2.71 dd (J=14.7 Hz/5.8 Hz, 1H, CH₂). | 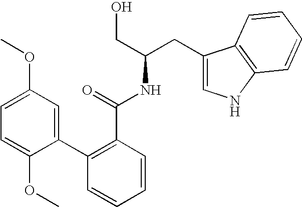 | | | |
| 70 | N-[(R)-1-(Hydroxymethyl)-2-(1H- indol-3-yl)ethyl]-2′,4.5′-tri- methoxy[1,1′-biphenyl]-2- carboxamide; D-Tryptophanol and 2′,4.5′-Trimethoxy[1,1′-biphenyl]- 2-carboxylic acid | 39 | (CD₃OD): 7.57 d (J=8.0 Hz, 1H, aryl); 7.31 d (J=8.0 Hz, 1H, aryl); 7.17 d (J=8.8 Hz, 1H, aryl); 7.07 dd (J=8.0 Hz/7.0 Hz, 1H, aryl); 7.03 s (1H, aryl); 7.02 dd (J=8.8 Hz/2.8 Hz, 1H, aryl); 6.98 d | |
| # (J=1.8 Hz, 1H, aryl); 6.97 dd (J=8.0 Hz/7.0 Hz, 1H, aryl); 6.85 d (J=8.6 Hz, 1H, aryl); 6.83 dd (J=8.6 Hz/2.5 Hz, 1H, aryl); 6.75 d (J=2.5 Hz, 1H, aryl); 4.15 m (1H, CH); 3.81 s (3H, OCH₃); 3.73 s (3H, OCH₃); 3.60 s (3H, OCH₃); 3.40 dd (J=10.9 Hz/4.5 Hz, 1H, CH₂OH); 3.31 m (1H, CH₂OH); 2.80 dd (J=14.4 Hz/7.6 Hz, 1H, CH₂); 2.72 dd (J=14.4 Hz/6.3 Hz, 1H, | | | | |
| # CH₂). | 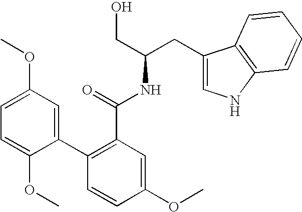 | | | |
| 71 | N-[(R)-1-(Hydroxymethyl)-2-(1H- indol-3-yl)ethyl]-2′,5′-dimethoxy- 6-methyl[1,1′-biphenyl]-3- carboxamide; D-Tryptophanol and 2′,4.5′-Trimethoxy[1,1′-biphenyl]- 2-carboxylic acid | 39 | (DMSO-d₆): 10.74 s (1H, NH); 8.10 d (J=8.1 Hz, 1H, NH); 7.73 dd (J=7.8 Hz/1.8 Hz, 1H, aryl); 7.63 d (J=8.0 Hz, 1H, aryl); 7.62 s (1H, aryl); 7.30 d (J=8.0 Hz, 1H, aryl); 7.30 d (J=7.8 Hz, 1H, | |
| # aryl); 7.10 d (J=1.8 Hz, 1H, aryl); 7.03 dd (J=8.0 Hz/7.0 Hz, 1H, aryl); 7.03 d (J=8.8 Hz, 1H, aryl); 6.95 dd (J=8.8 Hz/3.0 Hz, 1H, aryl); 6.94 dd (J=8.0 Hz/7.0 Hz, 1H, aryl); 6.69 d (J=3.0 Hz, 1H, aryl); 4.23 m (1H, CH); 3.74 s (3H, OCH₃); 3.64 s (3H, OCH₃); 3.50 m (1H, CH₂OH); 3.44 m (1H, CH₂OH); 2.99 dd (J=14.7 Hz/6.1 Hz, 1H, CH₂); 2.89 dd (J=14.7 Hz/7.8 Hz, 1H, | | | | |
| # CH₂). | 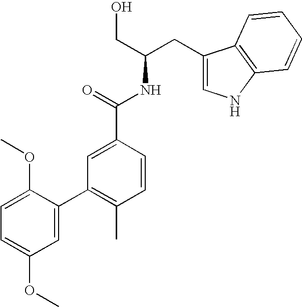 | | | |
| 72 | N-[(R)-1-(Hydroxymethyl)-2-(1H- indol-3-yl)ethyl]-2′,5′-dimethoxy- [1,1′-biphenyl]-4-carboxamide; D-Tryptophanol and 2′,5′-Dimethoxy[1,1′-biphenyl]-4- carboxylic acid | 39 | (CD₃OD): 7.78 d (J=8.6 Hz, 2H, aryl); 7.69 d (J=8.0 Hz, 1H, aryl); 7.55 d (J=8.6 Hz, 2H, aryl); 7.32 d (J=8.0 Hz, 1H, aryl); 7.12 d (J=1.8 Hz, 1H, aryl); 7.08 dd (J=8.0 Hz/7.0 Hz, 1H, aryl); 7.01 d | |
| # (J=8.6 Hz, 1H, aryl); 7.00 dd (J=8.0 Hz/7.0 Hz, 1H, aryl); 6.91 dd (J=8.6 Hz/3.0 Hz, 1H, aryl); 6.87 d (J=3.0 Hz, 1H, aryl); 4.46 m (1H, CH); 3.78 s (3H, OCH₃); 3.72 s (3H, OCH₃); 3.71 m (2H, CH₂OH); 3.16 dd (J=14.4 Hz/6.8 Hz, 1H, CH₂); 3.07 dd (J=14.4 Hz/7.1 Hz, 1H, CH₂). | 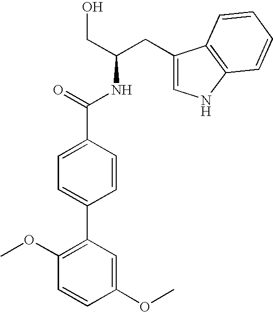 | | | |
| 73 | N-[(R)-1-(Hydroxymethyl)-2-(1H- indol-3-yl)ethyl]-2′,5′-dimethoxy- 2-methyl[1,1′-biphenyl]-4- carboxamide; D-Tryptophanol and 2′,5′-Dimethoxy-2-methyl[1,1′-bi- phenyl]-4-carboxylic acid | 39 | (CD₃OD): 7.69 d (J=8.0 Hz, 1H, aryl); 7.59 s (1H, aryl); 7.58 d (J=7.8 Hz, 1H, aryl); 7.33 d (J=8.0 Hz, 1H, aryl); 7.15 d (J=7.8 Hz, 1H, aryl); 7.13 d (J=1.8 Hz, 1H, aryl); 7.09 | |
| # dd (J=8.0 Hz/7.0 Hz, 1H, aryl); 7.01 dd (J=8.0 Hz/7.0 Hz, 1H, aryl); 6.98 d (J=9.1 Hz, 1H, aryl); 6.92 dd (J=9.1 Hz/3.0 Hz, 1H, aryl); 6.65 d (J=3.0 Hz, 1H, aryl); 4.45 m (1H, CH); 3.76 s (3H, OCH₃); 3.70 m (2H, CH₂OH); 3.66 s (3H, OCH₃); 3.16 dd (J=14.4 Hz/6.3 Hz, 1H, CH₂); 3.07 dd (J=14.4 Hz/6.6 Hz, 1H, CH₂); 2.12 s (3H, CH₃). | 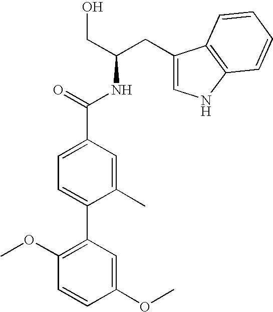 | | | |
| 74 | N-[(R)-1-(Hydroxymethyl)-2-(1H- indol-3-yl)ethyl]-3′,4′-dimethoxy- [1,1′-biphenyl]-2-carboxamide; D-Tryptophanol and 3′,4′-Dimethoxy[1,1′-biphenyl]-2- carboxylic acid | 39 | (CD₃OD): 7.58 d (J=8.0 Hz, 1H, aryl); 7.49-7.31 m (4H, aryl); 7.35 d (J=8.0 Hz, 1H, aryl); 7.09 dd (J=8.0 Hz/7.0 Hz, 1H, aryl); 7.00 d (J=1.8 Hz, 1H, aryl); 6.99 dd (J=8.0 Hz/7.0 Hz, 1H, aryl); 6.96 | |
| # d (J=2.1 Hz, 1H, aryl); 6.77 dd (J=8.3 Hz/2.1 Hz, 1H, aryl); 6.71 d (J=8.3 Hz, 1H, aryl); 4.21 m (1H, CH); 3.78 s (3H, OCH₃); 3.77 s (3H, OCH₃); 3.47 dd (J=11.1 Hz/5.3 Hz, 1H, CH₂OH); 3.40 dd (J=11.1 Hz/5.7 Hz, 1H, CH₂OH); 2.91 dd (J=14.5 Hz/7.0 Hz, 1H, CH₂); 2.78 dd (J=14.5 Hz/7.0 Hz, 1H, CH₂). | 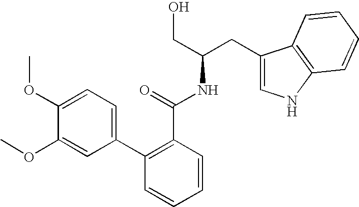 | | | |
| 75 | N-[(R)-1-(Hydroxymethyl)-2-(1H- indol-3-yl)ethyl]-3′,4,4′-tri- methoxy[1,1′-biphenyl]-2- carboxamide; D-Tryptophanol and 3′,4,4′-Trimethoxy[1,1′-biphenyl]- 2-carboxylic acid | 39 | (CD₃OD): 7.58 d (J=8.0 Hz, 1H, aryl); 7.34 d (J=8.0 Hz, 1H, aryl); 7.27 d (J=8.7 Hz, 1H, aryl); 7.09 dd (J=8.0 Hz/7.0 Hz, 1H, aryl); 7.01 d (J=2.6 Hz, 1H, aryl); 7.00 dd (J=8.7 Hz/2.6 Hz, 1H, | |
| # aryl); 6.99 dd (J=8.0 Hz/7.0 Hz, 1H, aryl); 6.92 d (J=1.8 Hz, 1H, aryl); 6.90 d (J=2.6 Hz, 1H, aryl); 6.74 dd (J=8.3 Hz/1.8 Hz, 1H, aryl); 6.70 d (J=8.3 Hz, 1H, aryl); 4.21 m (1H, CH); 3.79 s (3H, OCH₃); 3.78 s (3H, OCH₃); 3.76 s (3H, OCH₃); 3.47 dd (J=11.1 Hz/5.3 Hz, 1H, CH₂OH); 3.41 dd (J=11.1 Hz/5.7 Hz, 1H, CH₂OH); 2.91 dd (J=14.5 Hz/6.2 Hz, 1H, CH₂); | | | | |
| # 2.78 dd (J=14.5 Hz/7.0 Hz, 1H, CH₂). | 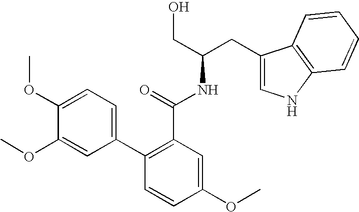 | | | |
| 76 | N-[(R)-1-(Hydroxymethyl)-2-(1H- indol-3-yl)ethyl]-3′,4′-dimethoxy- 6-methyl[1,1′-biphenyl]-3- carboxamide; D-Tryptophanol and 3′,4′-Dimethoxy-6-methyl[1,1′-bi- phenyl]-2-carboxylic acid | 39 | (CD₃OD): 7.65 d (J=8.0 Hz, 1H, aryl); 7.62 dd (J=7.9 Hz/1.9 Hz, 1H, aryl); 7.59 d (J=1.9 Hz, 1H, aryl); 7.31 d (J=7.9 Hz, 1H, aryl); 7.30 d (J=8.0 Hz, 1H, aryl); 7.10 d (J=1.8 Hz, | |
| # 1H, aryl); 7.05 dd (J=8.0 Hz/7.0 Hz, 1H, aryl); 7.02 d (J=8.1 Hz, 1H, aryl); 6.95 dd (J=8.0 Hz/7.0 Hz, 1H, aryl); 6.89 d (J=2.1 Hz, 1H, aryl); 6.85 dd (J=8.1 Hz/2.1 Hz, 1H, aryl); 4.44 m (1H, CH); 3.88 s (3H, OCH₃); 3.84 s (3H, OCH₃); 3.68 m (2H, CH₂OH); 3.13 dd (J=14.3 Hz/6.2 Hz, 1H, CH₂); 3.05 dd (J=14.3 Hz/7.2 Hz, 1H, CH₂).; 2.29 s (3H, CH₃) | 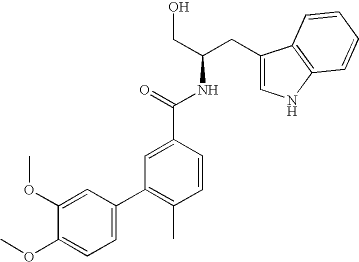 | | | |
| 77 | N-[(R)-1-(Hydroxymethyl)-2-(1H- indol-3-yl)ethyl]-3′,4′-dimethoxy- [1,1′-biphenyl]-4-carboxamide; D-Tryptophanol and 3′,4′-Dimethoxy[1,1′-biphenyl]-4- carboxylic acid | 39 | (CD₃OD): 7.81 d (J=8.7 Hz, 2H, aryl); 7.68 d (J=8.0 Hz, 1H, aryl); 7.65 d (J=8.7 Hz, 2H, aryl); 7.32 d (J=8.0 Hz, 1H, aryl); 7.23 d (J=1.7 Hz, 1H, aryl); 7.22 dd (J=9.0 Hz/1.7 Hz, 1H, aryl); 7.12 d | |
| # (J=1.8 Hz, 1H, aryl); 7.08 dd (J=8.0 Hz/7.0 Hz, 1H, aryl); 7.04 d (J=9.0 Hz, 1H, aryl); 7.00 dd (J=8.0 Hz/7.0 Hz, 1H, aryl); 4.47 m (1H, CH); 3.90 s (3H, OCH₃); 3.87 s (3H, OCH₃); 3.71 m (2H, CH₂OH); 3.16 dd (J=14.7 Hz/6.6 Hz, 1H, CH₂); 3.08 dd (J=14.7 Hz/7.3 Hz, 1H, CH₂). | 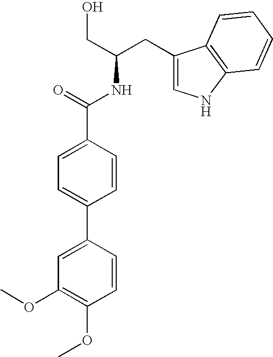 | | | |
| 78 | N-[(R)-1-(Hydroxymethyl)-2-(1H- indol-3-yl)ethyl]-3′,4′-dimethoxy- 2-methyl[1,1′-biphenyl]-4- carboxamide; D-Tryptophanol and 3′,4′-Dimethoxy-2-methyl[1,1′- biphenyl]-4-carboxylic acid | 39 | (CD₃OD): 7.69 d (J=8.0 Hz, 1H, aryl); 7.62 s (1H, aryl); 7.60 d (J=7.9 Hz, 1H, aryl); 7.33 d (J=8.0 Hz, 1H, aryl); 7.24d (J=7.9 Hz, 1H, aryl); 7.13 d (J=1.8 Hz, 1H, aryl); 7.08 dd (J=8.0 | |
| # Hz/7.0 Hz, 1H, aryl); 7.02 d (J=8.1 Hz, 1H, aryl); 7.01 dd (J=8.0 Hz/7.0 Hz, 1H, aryl); 6.86 dd (J=8.1 Hz/2.1 Hz, 1H, aryl); 6.80 d (J=2.1 Hz, 1H, aryl); 4.46 m (1H, CH); 3.87 s (3H, OCH₃); 3.84 s (3H, OCH₃); 3.71 m (2H, CH₂OH); 3.16 dd (J=14.7 Hz/7.0 Hz, 1H, CH₂); 3.07 dd (J=14.7 Hz/6.8 Hz, 1H, CH₂); 2.29 s (3H, CH₃). | 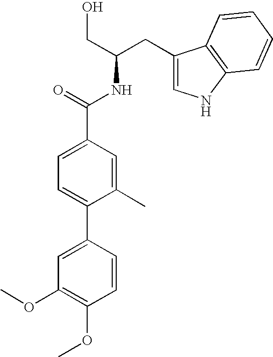 | | | |
| 79 | 3′-Fluoro-N-[(R)-1-(hydroxy- methyl)-2-(1H-indol-3-yl)ethyl]- 4′-methoxy[1,1′-biphenyl]-2- carboxamide; D-Tryptophanol and 3′-Fluoro-4′-methoxy]1,1′- biphenyl]-2-carboxylic acid | 39 | (CD₃OD): 7.60 d (J=8.0 Hz, 1H, aryl); 7.45 m (1H, aryl); 7.35 m (3H, aryl); 7.33 d (J=8.0 Hz, 1H, aryl); 7.12 dd (J=12.4 Hz/2.2 Hz, 1H, aryl); 7.10 dd (J=8.0 Hz/7.0 Hz, 1H, aryl); 7.06 d (J=1.8 | |
| # Hz, 1H, aryl); 7.00 dd (J=8.0 Hz/7.0 Hz, 1H, aryl); 6.89 ddd (J=8.5 Hz/2.2 Hz/1.1 Hz, 1H, aryl); 6.73 dd (J=8.7 Hz/8.5 Hz, 1H, aryl); 4.25 m (1H, CH); 3.79 s (3H, OCH₃); 3.54 dd (J=11.0 Hz/5.3 Hz, 1H, CH₂OH); 3.47 dd (J=11.0 Hz/5.8 Hz, 1H, CH₂OH); 2.98 dd (J=14.7 Hz/6.4 Hz, 1H, CH₂); 2.83 dd (J=14.7 Hz/7.5 Hz, 1H, CH₂). | 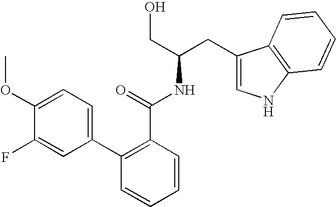 | | | |
| 80 | 3′-Fluoro-N-[(R)-1-(hydroxy- methyl)-2-(1H-indol-3-yl)ethyl]- 4,4′-dimethoxy[1,1′-biphenyl]-2- carboxamide; D-Tryptophanol and 3′-Fluoro-4,4′-dimethoxy]1,1′-bi- phenyl]-2-carboxylic acid | 39 | (CD₃OD): 7.60 d (J=8.0 Hz, 1H, aryl); 7.36 d (J=8.0 Hz, 1H, aryl); 7.24 d (J=8.5 Hz, 1H, aryl); 7.09 dd (J=8.0 Hz/7.0 Hz, 1H, aryl); 7.08 d (J=11.3 Hz, 1H, aryl); 7.06 d (J=1.8 Hz, | |
| # 1H, aryl); 7.00 dd (J=8.0 Hz/7.0 Hz, 1H, aryl); 7.00 dd (J=8.5 Hz/2.6 Hz, 1H, aryl); 6.86 d (J=2.6 Hz, 1H, aryl); 6.85 ddd (J=8.7 Hz/2.1 Hz/1.1 Hz, 1H, aryl); 6.72 dd (J=8.7 Hz/8.7 Hz, 1H, aryl); 4.25 m (1H, CH); 3.79 s (3H, OCH₃); 3.78 s (3H, OCH₃); 3.54 dd (J=11.0 Hz/5.3 Hz, 1H, CH₂OH); 3.48 dd (J=11.0 Hz/5.8 Hz, 1H, CH₂OH); 2.98 dd (J=14.7 Hz/6.3 Hz, 1H, CH₂); | | | | |
| # 2.84 dd (J=14.7 Hz/7.5 Hz, 1H, CH₂). | 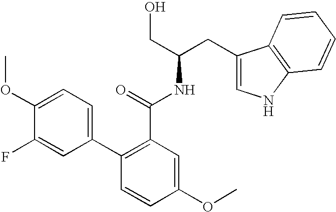 | | | |
| 81 | 3′-Fluoro-N-[(R)-1-(hydroxy- methyl)-2-(1H-indol-3-yl)ethyl]- 4′-methoxy[1,1′-biphenyl]-3- carboxamide; D-Tryptophanol and 3′-Fluoro-4′-methoxy]1,1′-bi- phenyl]-3-carboxylic acid | 39 | (CD₃OD): 7.90 dd (J=1.7 Hz/1.5 Hz, 1H, aryl); 7.71 d (J=7.9 Hz, 1H, aryl); 7.69 d (J=7.7 Hz, 1H, aryl); 7.68 d (J=8.0 Hz, 1H, aryl); 7.46 dd (J=7.9 Hz/7.7 Hz, 1H, aryl); 7.42 d (J=9.0 Hz, 1H, | |
| # aryl); 7.39 d (J=10.2 Hz, 1H, aryl); 7.32 d (J=8.0 Hz, 1H, aryl); 7.16 dd (J=9.0 Hz/8.5 Hz, 1H, aryl); 7.12 d (J=1.8 Hz, 1H, aryl); 7.08 dd (J=8.0 Hz/7.0 Hz, 1H, aryl); 6.98 dd (J=8.0 Hz/7.0 Hz, 1H, aryl); 4.47 m (1H, CH); 3.92 s (3H, OCH₃); 3.72 m (2H, CH₂OH); 3.16 dd (J=15.1 Hz/6.8 Hz, 1H, CH₂); 3.07 dd (J=15.1 Hz/7.5 Hz, 1H, CH₂). | 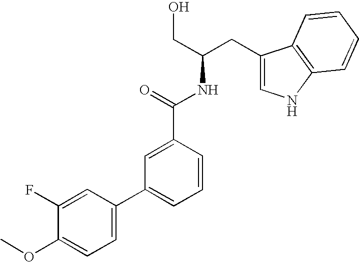 | | | |
| 82 | 3′-Fluoro-N-[(R)-1-(hydroxy- methyl)-2-(1H-indol-3-yl)ethyl]- 4′-methoxy-6-methyl[1,1′-bi- phenyl]-3-carboxamide; D-Tryptophanol and 3′-Fluoro-4′-methoxy-6-methyl- ]1,1′-biphenyl]-3-carboxylic acid | 39 | (CD₃OD): 7.65 d (J=8.0 Hz, 1H, aryl); 7.63 dd (J=7.9 Hz/2.1 Hz, 1H, aryl); 7.55 d (J=2.1 Hz, 1H, aryl); 7.31 d (J=8.0 Hz, 1H, aryl); 7.31 d (J=7.9 Hz, 1H, aryl); 7.15 dd | |
| # (J=9.0 Hz/8.3 Hz, 1H, aryl); 7.10 d (J=1.8 Hz, 1H, aryl); 7.06 dd (J=8.0 Hz/7.0 Hz, 1H, aryl); 7.06 m (2H, aryl); 6.96 dd (J=8.0 Hz/7.0 Hz, 1H, aryl); 4.43 m (1H, CH); 3.92 s (3H, OCH₃); 3.72 m (2H, CH₂OH); 3.13 dd (J=14.9 Hz/7.3 Hz, 1H, CH₂); 3.05 dd (J=14.9 Hz/6.6 Hz, 1H, CH₂); 2.28 s (3H, CH₃). | 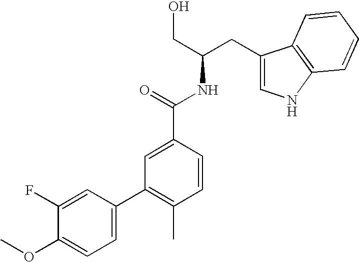 | | | |
| 83 | 3′-Fluoro-N-[(R)-1-(hydroxy- methyl)-2-(1H-indol-3-yl)ethyl]- 4′-methoxy[1,1′-biphenyl]-4- carboxamide; D-Tryptophanol and 3′-Fluoro-4′-methoxy]1,1′-bi- phenyl]-4-carboxylic acid | 39 | (CD₃OD): 7.81 d (J=8.7 Hz, 2H, aryl); 7.68 d (J=8.0 Hz, 1H, aryl); 7.63 d (J=8.7 Hz, 2H, aryl); 7.42 m (2H, aryl); 7.32 d (J=8.0 Hz, 1H, aryl); 7.16 dd (J=8.7 Hz/8.5 Hz, 1H, aryl); 7.12 d | |
| # (J=1.8 Hz, 1H, aryl); 7.08 dd (J=8.0 Hz/7.0 Hz, 1H, aryl); 7.00 dd (J=8.0 Hz/7.0 Hz, 1H, aryl); 4.47 m (1H, CH); 3.91 s (3H, OCH₃); 3.71 m (2H, CH₂OH); 3.16 dd (J=15.1 Hz/6.8 Hz, 1H, CH₂); 3.07 dd (J=15.1 Hz/7.7 Hz, 1H, CH₂). | 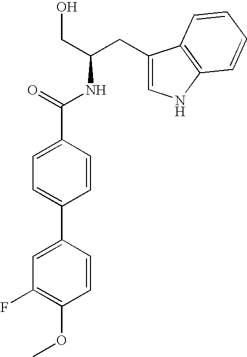 | | | |
| 84 | 3′-Fluoro-N-[(R)-1-(hydroxy- methyl)-2-(1H-indol-3-yl)ethyl]- 4′-methoxy-2-methyl[1,1′-bi- phenyl]-4-carboxamide; D-Tryptophanol and 3′-Fluoro-4′-methoxy-2-methyl- ]1,1′-biphenyl]-4-carboxylic acid | 39 | (CD₃OD): 7.68 d (J=8.0 Hz, 1H, aryl); 7.62 s (1H, aryl); 7.60 dd (J=9.2 Hz/1.3 Hz, 1H, aryl); 7.32 d (J=8.0 Hz, 1H, aryl); 7.22 d (J=9.2 Hz, 1H, aryl); 7.15 dd (J=8.7 Hz/8.5 | |
| # Hz, 1H, aryl); 7.12 d (J=1.8 Hz, 1H, aryl); 7.08 dd (J=8.0 Hz/7.0 Hz, 1H, aryl); 7.06 m (2H, aryl); 7.01 dd (J=8.0 Hz/7.0 Hz, 1H, aryl); 4.46 m (1H, CH); 3.91 s (3H, OCH₃); 3.71 m (2H, CH₂OH); 3.15 dd (J=14.7 Hz/7.0 Hz, 1H, CH₂); 3.05 dd (J=14.7 Hz/7.0 Hz, 1H, CH₂); 2.27 s (3H, CH₃). | 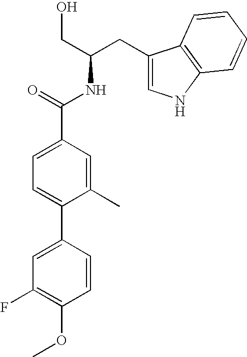 | | | |
| 85 | N-[(R)-1-(Hydroxymethyl)-2-(1H- indol-3-yl)ethyl]-3′,4-dimethoxy- [1,1′-biphenyl]-2-carboxamide; D-Tryptophanol and 3′,4′-Dimethoxy[1,1′-biphenyl]-2- carboxylic acid | 39 | (CD₃OD): 7.57 d (J=8.0 Hz, 1H, aryl); 7.33 d (J=8.0 Hz, 1H, aryl); 7.29 d (J=8.5 Hz, 1H, aryl); 7.16 dd (J=7.9 Hz/7.9 Hz, 1H, aryl); 7.08 dd (J=8.0 Hz/7.0 Hz, 1H, aryl); 7.02 dd (J=8.5 Hz/2.8 Hz, 1H, aryl); | |
| # 6.99 d (J=1.8 Hz, 1H, aryl); 6.98 dd (J=8.0 Hz/7.0 Hz, 1H, aryl); 6.91 m (1H, aryl); 6.89 d (J=2.8 Hz, 1H, aryl); 6.86 d (J=7.9 Hz, 1H, aryl); 6.82 d (J=7.9 Hz, 1H, aryl); 4.20 m (1H, CH); 3.78 s (3H, OCH₃); 3.75 s (3H, OCH₃); 3.45 dd (J=11.1 Hz/5.3 Hz, 1H, CH₂OH); 3.39 dd (J=11.1 Hz/5.7 Hz, 1H, CH₂OH); 2.88 dd (J=14.7 Hz/7.2 Hz, 1H, CH₂); 2.76 dd (J=14.7 Hz/7.3 | | | | |
| # Hz, 1H, CH₂). | 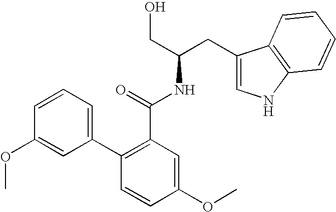 | | | |
| 86 | N-[(R)-1-(Hydroxymethyl)-2-(1H- indol-3-yl)ethyl]-3′-(1- methylethyl)[1,1′-biphenyl]-2- carboxamide D-Tryptophanol 3′-(1-Methylethyl)[1,1′-biphenyl]- 2-carboxylic acid | 39 | (CD₃OD): 7.53 d (J=8.0 Hz, 1H, aryl); 7.43 dd (J=7.2 Hz/7.2 Hz, 1H, aryl); 7.34 m (2H, aryl); 7.32 d (J=8.0 Hz, 1H, aryl); 7.30 dd (J=8.2 Hz/7.6 Hz, 1H, aryl); 7.22 s (1H, aryl); 7.16 m (2H, aryl); 7.11 m (1H, | |
| # aryl); 7.04 dd (J=8.0 Hz/7.0 Hz, 1H, aryl); 6.93 dd (J=8.0 Hz/7.0 Hz, 1H, aryl); 6.92d (J=1.8 Hz, 1H, aryl); 4.13 m (1H, CH); 3.37 dd (J=11.1 Hz/5.3 Hz, 1H, CH₂OH); 3.29 m (1H, CH₂OH); 2.82 m (1H, CH); 2.80 dd (J=15.1 Hz/7.0 Hz, 1H, CH₂); 2.70 dd (J=15.1 Hz/7.2 Hz, 1H, CH₂); 1.19 d (J=7.0 Hz, 6H, CH₃). | 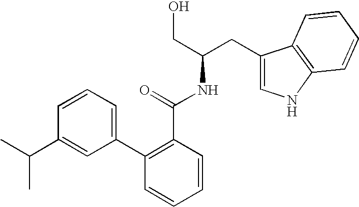 | | | |
| 87 | N-[(R)-1-(Hydroxymethyl)-2-(1H- indol-3-yl)ethyl]-2′,5′-dimethoxy- [1,1′-biphenyl]-3-carboxamide; D-Tryptophanol and 2′,5′-Dimethoxy[1,1′-biphenyl]-3- carboxylic acid | 39 | (CD₃OD): 7.85 dd (J=1.7 Hz/1.3 Hz, 1H, aryl); 7.68 d (J=8.0 Hz, 1H, aryl); 7.68 d (J=7.7 Hz, 1H, aryl); 7.63 d (J=7.7 Hz, 1H, aryl); 7.42 dd (J=7.7 Hz/7.7 Hz, 1H, aryl); 7.31 d (J=8.0 Hz, 1H, aryl); 7.12 | |
| # d (J=1.8 Hz, 1H, aryl); 7.06 dd (J=8.0 Hz/7.0 Hz, 1H, aryl); 7.00 d (J=9.6 Hz, 1H, aryl); 6.97 dd (J=8.0 Hz/7.0 Hz, 1H, aryl); 6.90 dd (J=9.6 Hz/3.0 Hz, 1H, aryl); 6.80 d (J=3.0 Hz, 1H, aryl); 4.46 m (1H, CH); 3.78 s (3H, OCH₃); 3.70 s (3H, OCH₃); 3.70 m (2H, CH₂OH); 3.15 dd (J=14.5 Hz/6.2 Hz, 1H, CH₂); 3.07 dd (J=14.5 Hz/7.2 Hz, 1H, CH₂). | 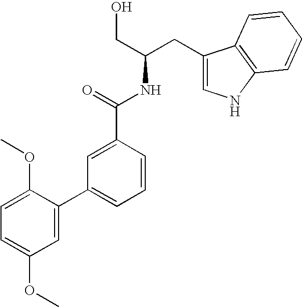 | | | |
| 88 | 3′,4′, 5′-Trifluoro-N-[(R)-1- (hydroxymethyl)-2-(1H-indol-3- yl)ethyl]-4-methoxy[1,1′-bi- phenyl]-2-carboxamide; D-Tryptophanol and 3′,4′,5′-Trifluoro-4-methoxy[1,1′- biphenyl]-2-carboxylic acid | 39 | (CD₃OD): 7.59 d (J=8.0 Hz, 1H, aryl); 7.34 d (J=8.0 Hz, 1H, aryl); 7.27 d (J=8.7 Hz, 1H, aryl); 7.09 dd (J=8.0 Hz/7.0 Hz, 1H, aryl); 7.07 d (J=1.6 Hz, 1H, aryl); 7.07 m | |
| # (2H, aryl); 7.01 dd (J=8.7 Hz/2.7 Hz, 1H, aryl); 7.00 dd (J=8.0 Hz/7.0 Hz, 1H, aryl); 6.79 d (J=2.7 Hz, 1H, aryl); 4.28 m (1H, CH); 3.75 s (3H, OCH₃); 3.55 m (2H, CH₂OH); 3.01 dd (J=14.5 Hz/5.8 Hz, 1H, CH₂); 2.87 dd (J=14.5 Hz/7.7 Hz, 1H, CH₂). | 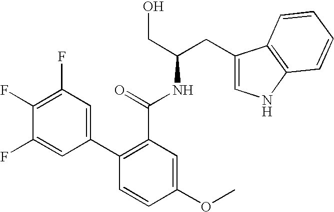 | | | |
| 89 | 3-(Benzofuran-2-yl)-N-[(R)-1- (hydroxymethyl)-2-(1H-indol-3- yl)ethyl]benzamide; D-Tryptophanol and 3-(Benzofuran-2-yl)benzoic acid | 39 | (CD₃OD): 8.24 s (1H, aryl); 8.01 d (J=7.9 Hz, 1H, aryl); 7.73 d (J=7.7 Hz, 1H, aryl); 7.69 d (J=8.0 Hz, 1H, aryl); 7.61 d (J=7.4 Hz, 1H, aryl); 7.53 d (J=7.4 Hz, 1H, aryl); 7.51 dd (J=7.9 Hz/7.7 Hz, 1H, aryl); 7.32 d (J=8.0 Hz, 1H, aryl); 7.30 dd | |
| # (J=8.3 Hz/7.4 Hz, 1H, aryl); 7.23 dd (J=8.3 Hz/7.4 Hz, 1H, aryl); 7.20 s (1H, aryl); 7.14 d (J=1.8 Hz, 1H, aryl); 7.08 dd (J=8.0 Hz/7.0 Hz, 1H, aryl); 7.00 dd (J=8.0 Hz/7.0 Hz, 1H, aryl); 4.49 m (1H, CH); 3.73 m (2H, CH₂OH); 3.17 dd (J=14.7 Hz/7.0 Hz, 1H, CH₂); 3.09 dd (J=14.7 Hz/7.3 Hz, 1H, CH₂). | 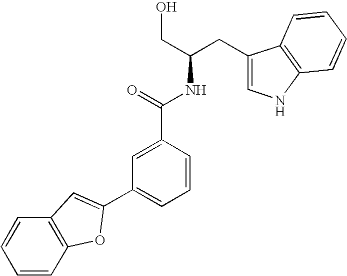 | | | |
| 90 | N-[(R)-1-(Hydroxymethyl)-2-(1H- indol-3-yl)ethyl]-3-(5-methoxy- benzofuran-2-yl)benzamide; D-Tryptophanol and 3-(5-Methoxybenzofuran-2-yl)- benzoic acid | 39 | (DMSO-d₆): 10.77 s (1H, NH); 8.37 d (J=8.3 Hz, 1H, NH); 8.33 s (1H, aryl); 8.02 d (J=7.8 Hz, 1H, aryl); 7.84 d (J=7.8 Hz, 1H, aryl); 7.68 d (J=8.0 Hz, 1H, aryl); 7.57 dd (J=7.8 Hz/7.8 Hz, 1H, aryl); 7.56 d (J=9.1 Hz, 1H, aryl); 7.43 s | |
| # (1H, aryl); 7.32 d (J=8.0 Hz, 1H, aryl); 7.16 s (1H, aryl); 7.05 dd (J=8.0 Hz/7.0 Hz, 1H, aryl); 6.98 dd (J=8.0 Hz/7.0 Hz, 1H, aryl); 6.93 dd (J=9.1 Hz/2.5 Hz, 1H, aryl); 4.28 m (1H, CH); 3.81 s (3H, OCH₃); 3.57 m (1H, CH₂OH); 3.53 m (1H, CH₂OH); 3.05 dd (J=14.4 Hz/5.8 Hz, 1H, CH₂); 2.96 dd (J=14.4 Hz/8.1 Hz, 1H, CH₂). | 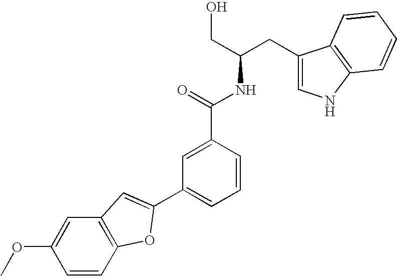 | | | |

### EXAMPLE 91

### N-[(R)-1-(Hydroxymethyl)-2-(1H-indol-3-yl)ethyl]-2-[(3,4,5-trimethoxyphenyl)methoxy]phenylpropanamide

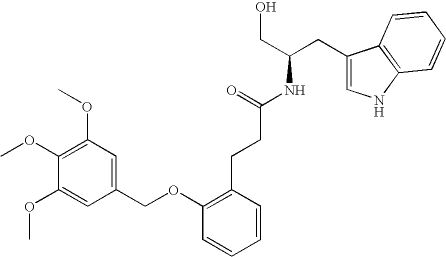

### 91a) Methyl 2-[(3,4,5-trimethoxyphenyl)methoxy]phenylpropanoate

0.38 mmol (68 mg) of methyl 2-hydroxyphenylpropanoate and 0.384 mmol (125 mg) of caesium carbonate were added to a solution of 0.38 mmol (100 mg) of 1-(bromomethyl)-3,4,5-trimethoxybenzene in 4 ml of acetonitrile. The mixture was heated to boiling for four hours. After cooling, the reaction mixture was diluted with saturated aqueous sodium hydrogen carbonate solution and extracted with ethyl acetate. The combined organic phases were washed with saturated aqueous sodium chloride solution, dried over sodium sulphate, filtered and concentrated in vacuo. Flash chromatography resulted in 122 mg of the target compound.

¹H-NMR (400 MHz, CDCl₃): δ [ppm]=7.18 d (J=7.3 Hz, 1H, aryl); 7.19 dd (J=8.3 Hz/7.6 Hz, 1H, aryl); 6.91 dd (J=8.3 Hz/7.3 Hz, 1H, aryl); 6.90 d (J=7.6 Hz, 1H, aryl); 6.67 s (2H, aryl); 5.02 s (2H, OCH₂); 3.88 s (6H, OCH₃); 3.86 s (3H, OCH₃); 3.64 s (3H, OCH₃); 3.02 t (J=7.9 Hz, 2H, CH₂); 2.67 t (J=7.9 Hz, 2H, CH₂).

### 91b) 2-[(3,4,5-Trimethoxyphenyl)methoxy]phenylpropanoic acid

In analogy to Example 39b), 112 mg of the title compound were obtained from 0.32 mmol (115 mg) of the compound prepared as in 91a) in 6.4 ml of methanol with 1.6 ml of a 2 molar aqueous sodium hydroxide solution.

¹H-NMR (400 MHz, CD₃OD): δ [ppm]=7.16 m (21H, aryl); 6.86 dd (J=7.5 Hz/7.3 Hz, 1H, aryl); 6.98 d (J=8.5 Hz, 1H, aryl); 6.78 s (2H, aryl); 5.05 s (2H, OCH₂); 3.83 s (6H, OCH₃); 3.75 s (3H, OCH₃); 2.96 t (J=7.9 Hz, 2H, CH₂); 2.60 t (J=7.9 Hz, 2H, CH₂).

### 91c) N-[(R)-1-(Hydroxymethyl)-2-(1H-indol-3-yl)ethyl]-2-[(3,4,5-trimethoxyphenyl)methoxy]phenylpropanamide

In analogy to Example 1e), 87 mg of the title compound were obtained from 0.27 mmol (95 mg) of the compound prepared as in 91 b) and 0.26 mmol (50 mg) of D-tryptophanol.

¹H-NMR (400 MHz, DMSO-d₆): δ [ppm]=10.74 s (1H, NH); 7.63 d (J=8.3 Hz, 1H, NH); 7.60 d (J=8.0 Hz, 1H, aryl); 7.31 d (J=8.0 Hz, 1H, aryl); 7.16 dd (J=7.9 Hz/7.7 Hz, 1H, aryl); 7.12 d (J=8.1 Hz, 1H, aryl); 7.05 s (1H, aryl); 7.04 dd (J=8.1 Hz/7.7 Hz, 1H, aryl); 7.04 d (J=7.9 Hz, 1H, aryl); 6.95 dd (J=8.0 Hz/7.0 Hz, 1H, aryl); 6.84 dd (J=8.0 Hz/7.0 Hz, 1H, aryl); 6.79 s (2H, aryl); 5.04 s (2H, OCH₂); 3.97 m (1H, CH); 3.76 s (6H, OCH₃); 3.65 s (3H, OCH₃); 3.33 m (2H, CH₂OH); 2.87 dd (J=14.5 Hz/7.9 Hz, 1H, CH₂); 2.83 m (2H, CH₂); 2.71 dd (J=14.5 Hz/7.0 Hz, 1H, CH₂); 2.38 m (2H, CH₂).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 92 | N-[(R)-1-(Hydroxy- methyl)-2- (1H-indol-3-yl)ethyl]- 4-[[(3,4,5- trimethoxyphenyl) methoxy]- methyl]benzamide; D-Tryptophanol and 4-[[(3,4,5- Trimethoxy- phenyl)methoxy]methyl]benzoic acid | 91 | | |
| # (DMSO-d₆): 10.74 broad s (1H, indole-NH); 8.13 d (J=8.3 Hz, 1H, amide); 7.83 D (J=8.4 Hz, 2H, aryl); 7.64 d (J=8.0 Hz, 1H, aryl); 7.42 d (J=8.4 Hz, 2H, aryl); 7.31 d (J=8.0 Hz, 1H, aryl); 7.12 d (J=2.1 Hz, 1H, aryl); 7.04 dd (J=8.0 Hz/7.0 Hz, 1H, aryl); 6.96 dd (J=8.0 Hz/ 7.0 Hz, 1H, aryl); 6.66 s | | | | |
| # (2H, aryl); 4.77 dd (J=5,7 Hz/5.7 Hz, OH); 4.57 s (2H, CH₂O); 4.47 S (2H, CH₂O); 4.23 m (1H, CHNH); 3.76 s (6H, OMe); 3.64 s (3H, OMe); 3.52 m (1H, CH₂OH); 3.48 m (1H, CH₂OH); 3.02 dd (J=15.1 Hz/5.8 Hz; 1H, CH₂- indole); 2.91 dd (J=15.1 Hz/7.7 Hz; 1H, CH₂- indole). | 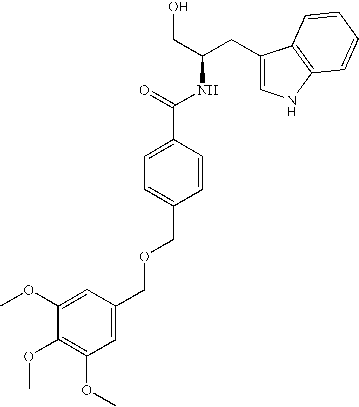 | | | |
| 93 | N-[(R)-1-(Hydroxy- methyl)-2- (1H-indol-3-yl)ethyl]- 3-[(3,4,5- trimethoxyphenyl)- methoxy]thio- phene-2-carboxamide; D-Tryptophanol and 3-[(3,4,5-Trimethoxy- phenyl)methoxy]- thiophene-2- carboxylic acid | 91 | (DMSO-d₆): 10.80 s (1H, | |
| # indole-NH); 7.68 d (J=5.5 Hz, 1H, aryl); 7.57 d (J=8.0 Hz, 1H, aryl); 7.55 d (J=8.1 Hz, 1H, amide); 7.31 d (J=8.0 Hz, 1H, aryl); 7.22 d (J=5.5 Hz; 1H, aryl); 7.05 dd (J=8.0 Hz/7.0 Hz, 1H, aryl); 6.98 d(J=2.3 Hz, 1H, indole); 6.95 dd (J=8.0/7.0 Hz, 1H, aryl); 6.76 s (2H, aryl); 5.21 d (J=11.7 Hz, 1H, | | | | |
| # CH₂O); 5.14 m (1H, CHNH); 5.13 d (J=11.7 Hz, 1H, CH₂O); 4.95 dd (J=5.3 Hz/5.3 Hz; 1H, OH); 3.93 dd (J=14.7 Hz/7,0 Hz, 1H, CH₂); 3.81 dd(J=14.7 Hz/6.4 Hz, 1H, CH₂); 3.73 s (6H, OMe); 3.63 s (3H, OMe); 3.44 m (1H, CH₂); 3.40 m (1H, CH₂). | 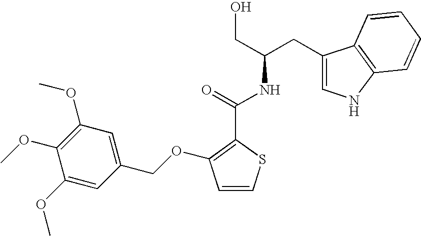 | | | |
| 94 | N-[(R)-1-(Hydroxy methyl)-2- (1H-indol-3-yl)- ethyl]-4-[(3,4,5- trimethoxy- phenyl)methoxy]- phenylacetamide; D-Tryptophanol and 4-[(3,4,5-Trimethoxy- phenyl)methoxy]- benzenessigsäure | 91 | (DMSO-d₆): 10.76 s (1H, | |
| # indole-NH); 7.82 d (J=8.1 Hz, 1H, amide); 7.58 d (J=8.0 Hz, 1H, aryl); 7.32 d (J=8.0 Hz, 1H, aryl); 7.08 d (J=8.7 Hz, 2H, aryl); 7.07 s (1H, aryl); 7.05 dd (J=8.0 Hz/7.0 Hz, 1H, aryl); 6.95 dd (J=8.0 Hz/7.0 Hz, 1H, aryl); 6.89 d (J=8.7 Hz; 2H; aryl); 6.76 s (2H, aryl); 4.97 s (2H, | | | | |
| # CH₂O); 4.73 dd (J=5.5 Hz/ 5.5 Hz, 1H, OH); 3.95 m (1H, CHNH); 3.77 s (6H, OMe); 3.65 s (3H, OMe); 3.36 m (2H, CH₂OH); 3.33 s (2H, CH₂); 2.90 dd (J=14.1 Hz/6.2 Hz, 1H, CH₂- indole); 2.74 dd (J=14.1 Hz/7.5 Hz, 1H,CH₂- indole). | 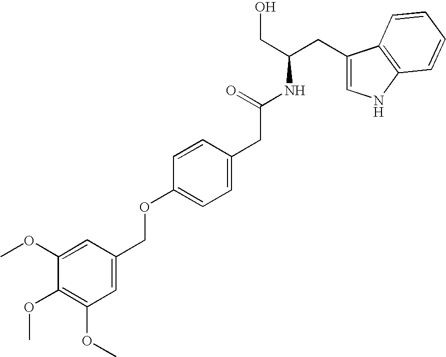 | | | |
| 95 | N-[(R)-1-(Hydroxy methyl)-2- (1H-indol-3-yl)- ethyl]-4-[(3,4,5- methoxyphenyl)- methoxy]- phenylpropanamide; D-Tryptophanol and 3-[3-((3,4,5-trimethoxy- phenyl)methoxy)- phenyl]-propionsäure | 91 | (DMSO-d₆): 10.75 s (1H, indole-NH); 7.68 d (J=8.3 Hz, 1H, amide); 7.60 d (J= | |
| # 8.0 Hz, 1H, aryl); 7.31 d (J=8.0 Hz, 1H, aryl); 7.17 dd (J=7.9 Hz/7.7 Hz, 1H, aryl); 7.07 d (J=2.1 Hz, 1H, indole); 7.05 dd (J=8.0 Hz/7.0 Hz, 1H, aryl); 6.87 s (1H, aryl); 6.85 dd (J=8.0 Hz/7.0 Hz, 1H, aryl); 6.82 dd (J=7.9 Hz/ | | | | |
| # 2.5 Hz, 1H, aryl); 6.76 dd (J=7.7 Hz/2.5 Hz, 1H, aryl); 6.75 s (2H, aryl); 4.96 s (2H, CH₂O); 4.70 dd (J=5.7 Hz/5.5 Hz, 1H, OH); 3.76 s (6H, OMe); 3.98 m (1H, CHNH); 3.65 s (3H, OMe); 3.33 m (2H, CH₂OH); 2.89 dd (J=14,1 Hz/6.6 Hz, 1H, CH₂); 2.74 m (2H, CH₂); 2.72 dd (J= | | | | |
| # 14.1 Hz/7.0 Hz, 1H, CH₂); 2.35 m (2H, CH₂). | 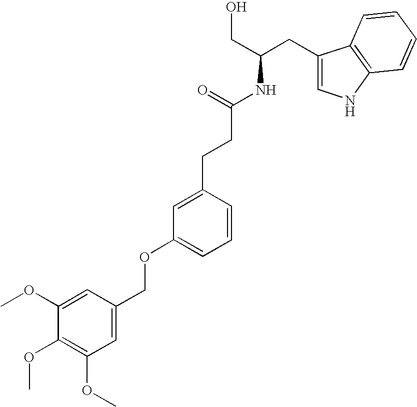 | | | |

### EXAMPLE 96

### 2-[2-(3,4-dimethoxyphenyl)ethyl]-N-[(R)-1-(hydroxymethyl)-2-(1H-indol-3-yl)ethyl]-6-methoxyquinoline-4-carboxamide

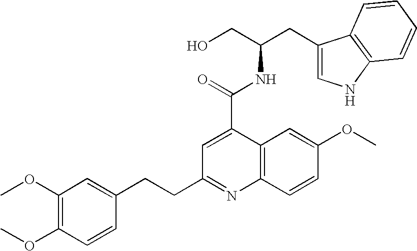

### 96a) 2-[(E)-2-(3,4-dimethoxyphenyl)ethenyl]-6-methoxyquinoline-4-carboxylic acid

4-methoxyisatin (4 g, 22.5 mmol) and (E)-3,4-dimethoxybenzylideneacetone (4.6 g, 22.5 mmol) were suspended in 30% strength aqueous KOH (20 ml) and heated under reflux for 8 hours. The reaction mixture was cooled and diluted with water, and the solid was filtered off. The residue on the filter was boiled three times with sodium hydroxide solution (1 N, 100 ml), and the combined mother liquors were acidified by adding acetic acid. A solid precipitates out of the solution after it has stood in a refrigerator overnight. The precipitate was filtered off, washed with water (100 ml) and dried in vacuo. 1.67 g (20% yield) of the title compound were obtained and could be employed in the next stage without further purification.

¹H-NMR (400 MHz, DMSO-d₆): δ [ppm]=8.18 s (1H); 8.08 d (J=2.6 Hz, 1H); 7.94 d (J=9.2 Hz, 1H); 7.71 d (J=16.2 Hz, 1H); 7.44 m (2H); 7.40 d (J=16.3 Hz, 1H); 7.22 d (J=8.1 Hz, 1H); 6.96 d (J=8.5 Hz, 1H); 3.86 s (3H); 3.81 s (3H); 3.76 s (3H).

### 96b) 2-[2-(3,4-dimethoxyphenyl)ethyl]-6-methoxyquinoline-4-carboxylic acid

2-[(E)-2-(3,4-dimethoxyphenyl)ethenyl]-6-methoxyquinoline-4-carboxylic acid (500 mg) was dissolved in methanol (10 ml) and aqueous sodium hydroxide solution (1 N, 5 ml) and concentrated to dryness in vacuo. The residue is dissolved in methanol (5 ml), a spatula tip of Pd/C is added, and hydrogenation is carried out under low pressure and at room temperature until no further uptake of hydrogen is to be observed. The catalyst was filtered off and the filtrate was concentrated in a rotary evaporator. Acidification with aqueous hydrochloric acid (1 N), removal of the precipitate by filtration and drying in vacuo resulted in 277 mg of the title compound which could be employed in the next stage without further purification.

¹H-NMR (400 MHz, DMSO-d₆): δ [ppm]=13.70 s broad (1H, acid); 8.07 d (J=2.8 Hz, 1H, aryl); 7.93 d (J=9.3 Hz, 1H, aryl); 7.83 s (1H, aryl); 7.42 dd (J=9.1 Hz/2.8 Hz; 1H, aryl); 6.85 d (J=1.8 Hz, 1H, aryl); 6.79 d (J=8.1 Hz, 1H, aryl); 6.72 dd (J=8.1 Hz/1.5 Hz, 1H, aryl); 3.85 s (3H, OMe); 3.66 s (6H, OMe); 3.18 m (2H, CH₂); 2.97 m (2H, CH₂).

### 96c) 2-[2-(3,4-dimethoxyphenyl)ethyl]-N-[(R)-1-(hydroxymethyl)-2-(1H-indol-3-yl)ethyl]-6-methoxyquinoline-4-carboxamide

In analogy to method 1e), D-tryptophanol (103 mg, 0.54 mmol) and the quinolinecarboxylic acid from method 98b) (100 mg, 0.27 mmol) were reacted to give the title compound and purified by recrystallization from ethanol. 122 mg of the title compound were obtained.

¹H-NMR (400 MHz, DMSO-d₆): δ [ppm]=10.80 s (1H, indole-NH); 8.47 d (J=8.7 Hz, 1H, amide); 7.86 d (J=9.9 Hz, 1H, aryl); 7.62 d (J=7.7 Hz, 1H, aryl); 7.30 m (4H, aryl); 7.17 d (J=1.9 Hz, 1H, aryl); 7.03 dd (J=7.0 Hz/7.0 Hz, 1H, aryl); 6.94 dd (J=7.0 Hz/7.0 Hz, 1H, aryl); 6.87 d (J=1.5 Hz, 1H, aryl); 6.80 m (2H, aryl); 4.84 dd (J=5.5 Hz/5.5 Hz, 1H, OH); 4.35 m (1H, CH₂); 3.68 s (3H, OMe); 3.67 s (3H, OMe); 3.66 s (3H, OMe); 3.54 dd (J=5.6 Hz/5.6 Hz, 2H, CH₂); 3.10 m (2H, CH₂); 2.92 m (4H, CH₂).

| | | Method | | |
|---|---|---|---|---|
| | Product | analogous | ¹H-NMR (400 MHz) δ | |
| Ex. | reagents | to | [ppm] | Structure |
| 97 | 2-(6-Methoxy-naphthalen-2- yl)quinoline-4-carboxylic acid [(R)-1-hydroxymethyl-2-(1H- indol-3-yl)ethyl]amide; (D)-Tryptophanol and 2-(6-Methoxy-naphthalen-2- yl)quinoline-4-carboxylic acid | 13 | (DMSO-d₆): 10.85 s (1H); 8.67 s (1H); 8.64 d (J=8.3 Hz, 1H); 8.33 dd (J=2.0 | |
| # Hz/8.8 Hz, 1H); 8.09-7.96 m (4H); 7.84 d (J=8.3 Hz, 1H); 7.76 m (1H); 7.66 d (J=7.8 Hz, 1H); 7.48 m (1H); 7.37 m (2H); 7.24 dd (J=2.5 Hz/9.1 Hz, 1H); 7.18 m (1H); 7.06 m (1H); 6.95 m (1H); 4.39 m (1H); 3.89 s (3H); 3.55 m (2H); 3.08 dd (J=5.8 Hz/14.7 Hz, 1H); 2.92 dd (J=8.3 Hz/14.4 Hz, 1H). | 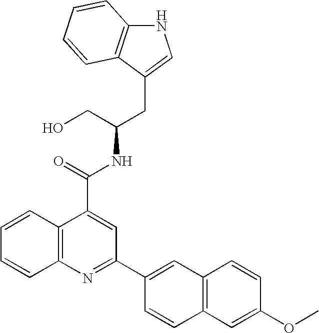 | | | |
| 98 | 6-Methoxy-2-(3- methoxyphenyl)quinoline-4- carboxylic acid [(R)-1- hydroxymethyl-2-(1H-indol-3- yl)ethyl]amide; (D)-Tryptophanol and 6-Methoxy-2-(3- methoxyphenyl)quinoline-4- carboxylic acid | 13 | (DMSO-d₆): 10.81 s (1H); 8.63 d (J=8.6 Hz, 1H); 7.98 d (J=9.1 Hz, 1H); 7.89 s (1H); 7.75 s (1H); | |
| # 7.71 d (J=7.8 Hz, 1H); 7.64 d (J=7.8 Hz, 1H); 7.45 m (3H); 7.31 d (J=8.1 Hz, 1H); 7.18 s (1H); 7.05 m (1H); 6.93 m (1H); 4.89 m (1H); 4.38 m (1H); 3.84 s (3H); 3.71 s (3H); 3.57 m (2H); 3.03 dd (J=5.6 Hz/14.7 Hz, 1H); dd (J=8.1 Hz/14.7 Hz, 1H). | 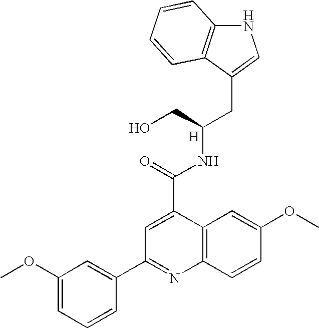 | | | |
| 99 | 2-(4-Fluoro-3-methoxyphenyl)- 6-methoxyquinoline-4- carboxylic acid [(R)-2-hydroxy- 1-(1H-indol-3- ylmethyl)ethyl]amide; (D)-Tryptophanol and 2-(4-Fluoro-3-methoxyphenyl)- 6-methoxyquinoline-4- carboxylic acid | 13 | (DMSO-d₆): 10.80 s (1H); 8.62 d (J=8.7 Hz, 1H); 7.96 m (2H); 7.90 s (1H); | |
| # 7.69 m (1H); 7.61 d (J=7.9 Hz, 1H); 7.42 m (3H); 7.19 s (1H); 7.03 m (1H); 6.93 m (1H); 4.87 m (1H); 4.37 m (1H); 3.96 s (3H); 3.71 m (3H); 3.57 m (2H); 3.01 m (1H); 2.91 m (1H). | 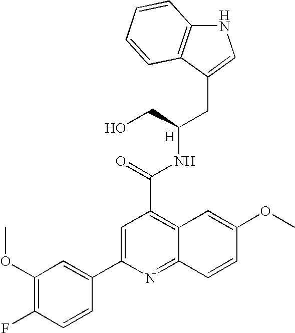 | | | |
| 100 | 2-(3-Iodo-4-methoxyphenyl)-6- methoxyquinoline-4-carboxylic acid [(R)-2-hydroxy-1-(1H-indol- 3-ylmethyl)ethyl]amide; (D)-Tryptophanol and 2-(3-Iodo-4-methoxyphenyl)-6- methoxyquinoline-4- carboxylic acid | 13 | (DMSO-d₆): 10.79 s (1H); 8.62 m (1H); 8.15 dd (J= | |
| # 2.1 Hz/8.5 Hz, 1H); 7.95 d (J=9.6 Hz, 1H); 7.84 s (1H); 7.63 d (J=7.9 Hz, 1H); 7.40 s (1H); 7.30 m (2H); 7.18 m (2H); 7.04 m (1H); 6.94 m (1H); 4.87 m (1H); 4.36 m (1H); 3.90 s (3H); 3.70 s (3H); 3.57 m (2H); 3.01 m (1H); 2.90 m (1H). | 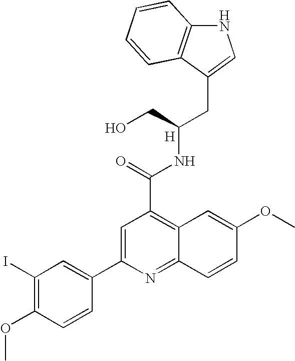 | | | |
| 101 | 2-(3-Hydroxyphenyl)-6- methoxyquinoline-4-carboxylic acid [(R)-1-hydroxymethyl-2- (1H-indol-3-yl)ethyl]amide; (D)-Tryptophanol and 2-(3-Hydroxyphenyl)-6- methoxyquinoline-4- carboxylic acid | 13 | (DMSO-d₆): 10.79 s (1H); 9,60 s (1H); 8.62 d (J=8.7 Hz, 1H); 7.94 d (J=9.6 | |
| # Hz, 1H); 7.82 S (1H); 7.64 m (2H); 7.51 d (J=8.1 Hz, 1H); 7.41 m (2H); 7.32 m (2H); 7.18 s (1H); 7.03 m (1H); 6.93 m (1H); 6.87 m (1H); 4.88 m (1H); 4.38 m (1H); 3.71 s (3H); 3.56 m (2H); 3.01 m (1H); 2.93 m (1H). | 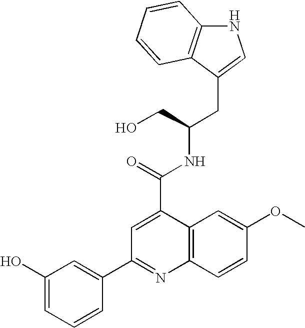 | | | |
| 102 | 2-(4-Hydroxy-3,5- dimethoxyphenyl)-6- methoxyquinoline-4-carboxylic acid [(R)-1-hydroxymethyl-2- (1H-indol-3-yl)ethyl]amide; (D)-Tryptophanol and 2-(4-Hydroxy-3,5- dimethoxyphenyl)-6- methoxyquinoline-4- carboxylic acid | 13 | (DMSO-d₆): 10.79 s (1H); 8.78 s (1H); 8.61 d (J=8.8 | |
| # Hz, 1H); 7.90 m (2H); 7.63 d (J=7.8 Hz, 1H); 7.47 s (2H); 7.39 m (3H); 7.30 d (J=8.1 Hz, 1H); 7.19 s (1H); 7.02 m (1H); 6.93 m (1H); 4.86 m (1H); 4.37 m (1H); 3.87 s (6H); 3.69 s (3H); 3.57 m (2H); 3.02 dd (J=5.8 Hz/15.1 Hz, 1H); 2.91 dd (J=7.6 Hz/14.4 Hz, 1H). | 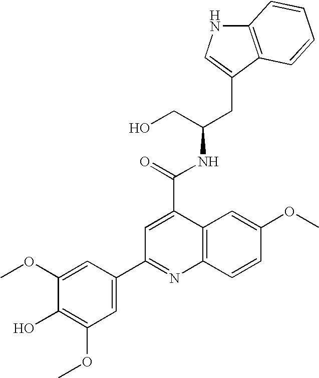 | | | |
| 103 | 2-(3,5-Difluoro-4- methoxyphenyl)-6- methoxyquinoline-4-carboxylic acid [(R)-2-hydroxy-1-(1H-indol- 3-ylmethyl)ethyl]amide; (D)-Tryptophanol and 2-(3,5-Difluoro-4- methoxyphenyl)-6- methoxyquinoline-4- carboxylic acid | 13 | (DMSO-d₆): 10.80 s (1H); 8.59 d (J=8.7 Hz, 1H); 7.93 m (4H); 7.63 (d (J= | |
| # 7.9 Hz, 1H); 7.41 m (2H); 7.31 d (J=7.9 Hz, 1H); 7.18 s (1H); 7.02 m (1H); 6.92 m (1H); 4.87 m (1H); 4.37 m (1H); 3.99 s (3H); 3.70 s (3H); 3.57 m (2H); 3.01 dd(J=6.4 Hz/14.7 Hz, 1H); 2.89 dd (J=8.1 Hz/14.3 Hz, 1H). | 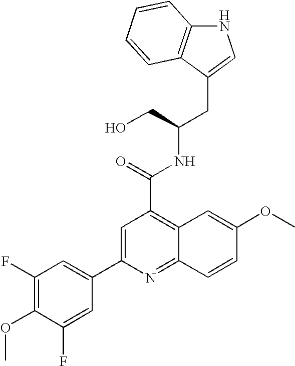 | | | |
| 104 | 2-(3-Ethylphenyl)-6- methoxyquinoline-4-carboxylic acid [(R)-1-hydroxymethyl-2- (1H-indol-3-yl)ethyl]amide; (D)-Tryptophanol and 2-(3-Ethylphenyl)-6- methoxyquinoline-4- carboxylic acid | 13 | (DMSO-d₆): 10.80 s (1H); 8.61 d (J=8.6 Hz, 1H); 7.96 m (4H); 7.86 s (1H); | |
| # 7.63 d (J=7.8 Hz, 1H); 7.41 m (4H); 7.32 d (J=8.1 Hz, 1H); 7.18 s (1H); 7.03 m (1H); 6.93 m (1H); 4.87 m (1H); 4.37 m (1H); 3.71 s (3H); 3.57 m (2H); 2.95 dd (J=5.6 Hz/14.7 Hz, 1H); 2.91 dd (J=8.1 Hz/14.7 Hz, 1H); 2.71 m (2H); 1.21 m (3H). (2H); 1.21 m (3H). | 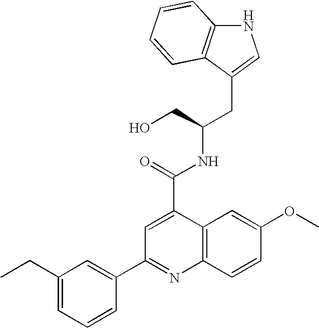 | | | |
| 105 | 2-(3-Fluoro-4-methoxyphenyl)- 6-methoxyquinoline-4- carboxylic acid [(R)-2-hydroxy- 1-(1H-indol-3- ylmethyl)ethyl]amide; (D)-Tryptophanol and 2-(3-Fluoro-4-methoxyphenyl)- 6-methoxyquinoline-4- carboxylic acid | 13 | (DMSO-d₆): 10.80 s (1H); 8.59 d (J=8.7 Hz, 1H); | |
| # 8.00 m (3H); 7.85 s (1H); 7.63 d (J=7.7 Hz, 1H); 7.35 m (4H); 7.18 d (J=2.1 Hz, 1H); 7.04 m (1H); 6.93 m (1H); 4.86 m (1H); 4.37 m (1H); 3.91 s (3H); 3.70 s (3H); 3.57 m (2H); 3.02 m (1H); 2.93 m (1H). | 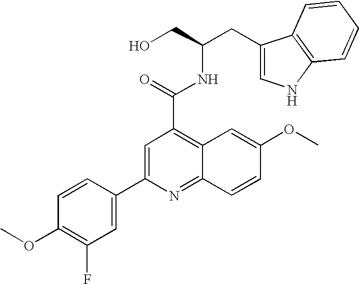 | | | |
| 106 | 2-(3-Fluoro-4-methoxyphenyl)- 6-methylquinoline-4-carboxylic acid [(R)-2-hydroxy-1-(1H-indol- 3-ylmethyl)ethyl]amide; (D)-Tryptophanol and 2-(3-Fluoro-4-methoxyphenyl)- 6-methylquinoline-4- carboxylic acid | 13 | (DMSO-d₆): 10.83 s (1H); 8.56 d (J=8.7 Hz, 1H); 8.06 dd (J=2.1Hz/13.0 | |
| # Hz, 1H); 7.98 d (J=9.8 Hz, 1H); 7.91 d (J=8.5 Hz, 1H); 7.84 s (1H); 7.62 m (3H); 7.32 m (2H); 7.18 s (1H); 7.05 m (1H); 6.94 m (1H); 4.88 m (1H); 4.38 m (1H); 3.91 s (3H); 3.57 m (2H); 3.03 m (1H); 2.90 m (1H); 2.38 s (3H). | 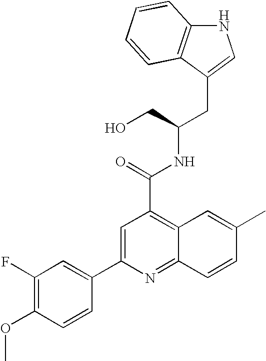 | | | |
| 107 | 6-Methyl-2-(3,4,5- trimethoxyphenyl)quinoline-4- carboxylic acid [(R)-1- hydroxymethyl-2-(1H-indol-3- yl)ethyl]amide; | 13 | (DMSO-d₆): 10.81 s (1H); 8.58 d (J=8.3 Hz, 1H); 7.93 m (2H); 7.62 m (2H); 7.58 dd (J=1.8 Hz/8.6 Hz, 1H); 7.48 s (2H); 7.32 d (J=8.1 Hz, 1H); 7.18 s (1H); 6.95 m (1H); 6.93 m | |
| # (1H); 4.86 m (1H); 4.36 m (1H); 3.89 s (6H); 3.72 s (3H); 3.57 m (2H); 3.04 m (1H); 2.91 m (1H); 2.65 s (3H). | 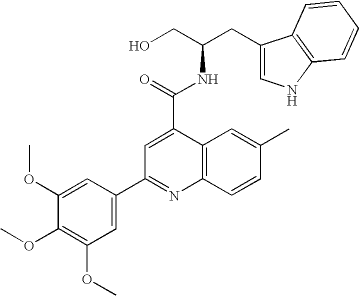 | | | |
| 108 | 6-Bromo-2-(2,4-dimethyl- thiazol-5-yl)quinoline-4- carboxylic acid [(R)-2-hydroxy- 1-(1H-indol-3- ylmethyl)ethyl]amide; (D)-Tryptophanol and 6-Bromo-2-(2,4-dimethyl- thiazol-5-yl)quinoline-4- carboxylic acid | 13 | (DMSO-d₆): 10.81 s (1H); 8.72 d (J=8.7 Hz, 1H); 8.15 s (1H); 7.88 s (2H); | |
| # 7.63 s (1H); 7.58 d (J=7.9 Hz, 1H); 7.32 d (J=8.1 Hz, 1H); 7.17 d(J=2.1 Hz, 1H); 7.02 m (1H); 6.92 m (1H); 4.88 m (1H); 4.33 m (1H); 3.55 m (2H); 3.02 dd (J=5.3 Hz/14.7 Hz, 1H); 2.90 dd (J=8.3 Hz/ 14.7 Hz, 1H); 2.64 s (3H); 2.62 s (3H). | 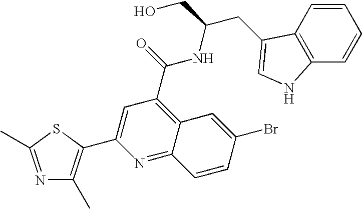 | | | |
| 109 | 2-(7-Methoxybenzofuran-2-yl)- 6-trifluoromethoxyquinoline-4- carboxylic acid [(R)-2-hydroxy- 1-(1H-indol-3- ylmethyl)ethyl]amide; (D)-Tryptophanol and 2-(7-Methoxybenzofuran-2-yl)- 6-trifluoromethoxyquinoline-4- carboxylic acid | 13 | (DMSO-d₆): 10.79 s (1H); 8.81 d (J=8.9 Hz, 1H); 8.23 d (J=9.2 Hz, 1H); | |
| # 8.07 s (1H); 7.93 s (1H); 7.80 m (2H); 7.62 d (J=7.7 Hz, 1H); 7.33 m (3H); 7.18 s (1H); 7.03 m (3H); 4.90 m (1H); 4.36 m (1H); 3.99 s (3H); 3.56 m (2H); 3.02 m (1H); 2.92 m (1H). | 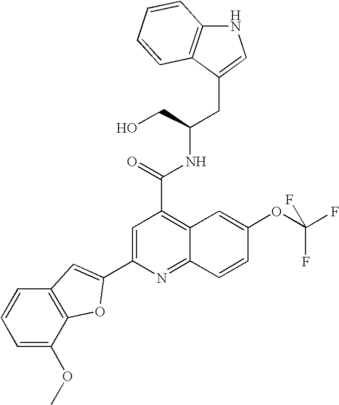 | | | |
| 110 | 2-(3-Fluoro-4-methoxyphenyl)- 6-iodoquinoline-4-carboxylic acid [(R)-2-hydroxy-1-(1H-indol- 3-ylmethyl)ethyl]amide; (D)-Tryptophanol and 2-(3-Fluoro-4-methoxyphenyl)- 6-iodoquinoline-4- carboxylic acid | 13 | (DMSO-d₆): 10.80 s (1H); 8.68 d (J=8.5 Hz, 1H); 8.44 s (1H); 8.00 m (3H); | |
| # 7.91 s (1H); 7.80 d (J=8.9 Hz, 1H); 7.65 d (J=7,9 Hz, 1H); 7.33 m (2H); 7.18 s (1H); 7.04 m (1H); 6.94 m (1H); 4.34 m (1H); 3.92 s (3H); 3.57 m (2H); 3.03 m (1H); 2.92 m (1H). | 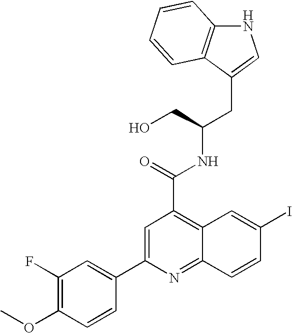 | | | |
| 111 | 2-(3-Fluoro-4-methoxyphenyl)- 6-trifluoromethoxyquinoline-4- carboxylic acid [(R)-2-hydroxy- 1-(1H-indol-3- ylmethyl)ethyl]amide; (D)-Tryptophanol and 2-(3-Fluoro-4-methoxyphenyl)- 6-trifluoromethoxyquinoline-4- carboxylic acid | 13 | (DMSO-d₆): 10.84 s (1H); 8.76 d (J=8.5 Hz); 8.76 d | |
| # (J=8.5 Hz, 1H); 8.21 d (J=9.23 Hz, 1H); 8.16-8.04 m (4H); 7.81 dd (J=2.8 Hz/8.5 Hz, 1H); 7.68 d (J=7.7 Hz, 1H); 7.37 m (2H); 7.21 d (J=2.1 Hz, 1H); 7.07 m (1H); 6.97 m (1H); 4.91 m (1H); 4.38 m (1H); 3.97 s (3H); 3.61 m (2H); 3.09 dd (J=6.2 Hz/ 14.7 Hz, 1H); 2.94 dd (J= | | | | |
| # 7.5 Hz/14.5 Hz, 1H). | 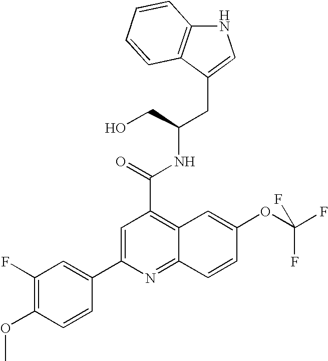 | | | |
| 112 | 2-(3-Fluoro-4-methoxyphenyl)- 6,8-dimethylquinoline-4- carboxylic acid [(R)-2-hydroxy- 1-(1H-indol-3- ylmethyl)ethyl]amide; (D)-Tryptophanol and 2-(3-Fluoro-4-methoxyphenyl)- 6,8-dimethylquinoline-4- carboxylic acid | 13 | (DMSO-d₆): 10.86 s (1H); 8.55 d (J=8.5 Hz, 1H); 8.14 dd (J=2.1 Hz/15.3 | |
| # Hz, 1H); 8.04 d (J=8.7 Hz, 1H); 7.87 s (1H); 7.69 d(J=7.9 Hz, 1H); 7.49 d (J=2.3 Hz, 1H); 7.37 d (J=8.7 Hz, 2H); 7.22 s (1H); 7.10 m (1H); 6.99 m (1H); 4.90 m (1H); 4.40 m (1H); 3.96 s (3H); 3.60 m (2H); 3.08 m (1H); 2.95 m (1H); 2.76 s (3H); 2.38 s (3H). | 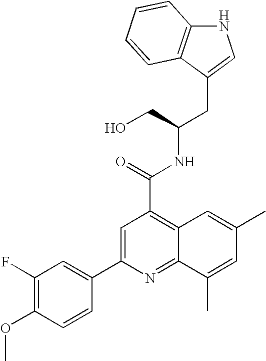 | | | |

### EXAMPLE 113

### 2-(3,4-Dimethoxyphenyl)-6-methoxy-3-methylquinoline-4-carboxylic acid [(R)-1-hydroxymethyl-2-(1H-indol-3-yl)ethyl]amide;

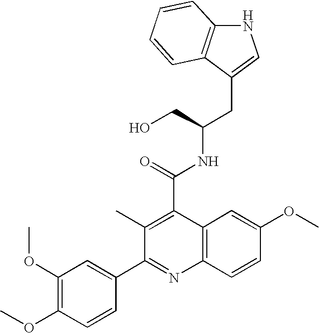

### 113a) 2-(3,4-Dimethoxyphenyl)-6-methoxy-3-methylquinoline-4-carboxylic acid

3′,4′-Dimethoxy-1-phenylpropiophenone (1.5 g) and 5-methoxyisatin (1.4 g) were heated together in aqueous 30% strength potassium hydroxide solution (20 ml) under reflux overnight. The reaction mixture was added to water and the remaining residue was filtered off with suction. The filtrate was acidified with glacial acetic acid and placed in a refrigerator overnight. The precipitated reaction product was filtered off, dried in vacuo and employed without further purification in the next stage (yield 34%).

(DMSO-d₆): 7.90 d (J=9.2 Hz); 7.39 dd (J=9.2 Hz/2.8 Hz, 1H); 7.04 m (4H); 3.85 s (3H); 3.79 s (3H); 3.76 s (3H); 2.35 s (3H).

### 113b) 2-(3,4-Dimethoxyphenyl)-6-methoxy-3-methylquinoline-4-carboxylic acid [(R)-1-hydroxymethyl-2-(1H-indol-3-yl)ethyl]amide

The quinolinecarboxylic acid from the previous stage (200 mg) was stirred together with (D)-tryptophanol (108 mg), HOBt (87 mg), EDC (109 mg) and diisopropylethylamine (0.099 ml) in DMF (10 ml) at room temperature overnight. The mixture was added to water and stirred for 10 minutes, and the precipitate was filtered off. The crude product was purified by column chromatography using Flashmasters and crystallized from diisopropyl ether. The title compound is obtained in 30% yield (90 mg).

(DMSO-d₆): 10.76 s (1H); 8.56 d (J=8.9 Hz, 1H); 7.83 d (J=9.2 Hz, 1H); 7.59 d (J=7.7 Hz, 1H); 7.27 m (3H); 7.02 m (5H); 4.90 m (1H); 4.47 m (1H); 3.79 s (6H); 3.56 m (2H); 2.96 m (1H); 2.69 m (1H); 2.05 s (3H).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 114 | 6-Amino-2-(3-fluoro-4- methoxyphenyl)quinoline-4- carboxylic acid [(R)-2-hydroxy- 1-(1H-indol-3- ylmethyl)ethyl]amide; 2-(3-Fluoro-4-methoxyphenyl)- 6-nitroquinoline-4-carboxylic acid [(R)-2-hydroxy-1-(1H-indole- 3-ylmethyl)ethyl]amide | 20 | (DMSO-d₆): 10.82 s (1H); 8.43 d (J=8.3 Hz, 1H); 7.94 d (J=2.3 Hz/13.1 Hz | |
| # 1H); 7.84 d (J=9.4 Hz, 1H); 7.70 m (2H); 7.19 s (1H); 7.14 m (1H); 7.03 m (1H); 6.96 m (1H); 5.69 s (2H); 4.82 (m, 1H); 4.28 m (1H); 3.89 s (3H); 3.55 m (2H); 3.03 m (1H); 2.96 m (1H). | 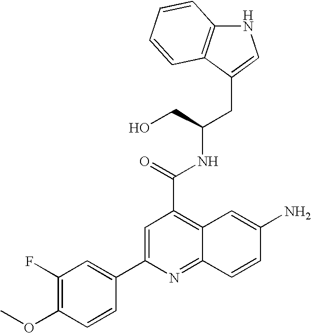 | | | |

### EXAMPLE 115

### 2-(4,6-Dimethoxybenzofuran-2-yl)-N-[(R)-1-(hydroxymethyl)-2-(1H-indol-3-yl)ethyl]-6-methoxyquinoline-4-carboxamide

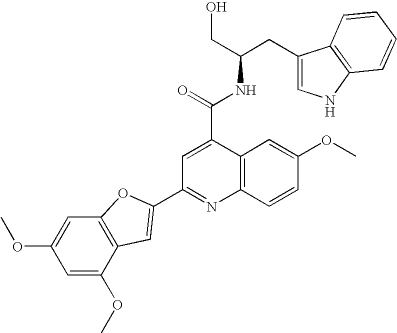

### 115a) 1-(4,6-Dimethoxybenzofuran-2-yl)ethanone

4,6-Dimethoxysalicylaldehyde (500 mg), 1-chloro-2-propanone (241 μl), potassium carbonate (379 mg) were stirred in 2-butanone (20 ml) under a nitrogen atmosphere at 90° C. for 8 hours. The reaction mixture was diluted with water and extracted with ethyl acetate, and the combined organic phases were washed with saturated aqueous NaCl solution. The solvent was distilled out in vacuo, and the crude product was purified by Flashmaster chromatography. The title compound was obtained in 29% yield (174 mg).

(CDCl₃): 7.53 s (1H); 6.64 m (1H); 6.32 s (1H); 3.91 s (3H); 3.86 s (3H); 2.53 s (3H).

### 115b) 2-(4,6-Dimethoxybenzofuran-2-yl)-6-methoxyquinoline-4-carboxylic acid

1-(4,6-Dimethoxybenzofuran-2-yl)-ethanone (169 mg), 5-methoxyisatin (136 mg) were stirred together with potassium hydroxide solution (30% strength in water, 2.7 ml) under a nitrogen atmosphere at 80° C. for 8 hours. The reaction mixture was added to 150 ml of water and, while cooling in ice, acidified with 70% strength acetic acid until a pH of 5-6 was reached. After 30 minutes, stirring with n-butanol/ethyl acetate (1:1, 20 ml) and back-extraction with ethyl acetate were carried out. The combined organic phases were washed with saturated aqueous NaCl solution. The solvent was distilled out in vacuo. Crystallization from dichloromethane/methanol results in the title compound in 78% yield (227 mg).

(DMSO-d₆): 8.39 s (1H); 8.10 s (1H); 8.00 m (J=9.3 Hz, 1H); 7.64 s (1H); 7.48 m (1H); 6.95 s (1H); 6.44 s (1H); 3.88 s (6H); 3.81 s (3H).

### 115c) 2-(4,6-Dimethoxybenzofuran-2-yl)-N-[(R)-1-(hydroxymethyl)-2-(1H-indol-3-yl)ethyl]-6-methoxyquinoline-4-carboxamide

The quinolinecarboxylic acid (120 mg) was converted into the title compound in 64% yield (93 mg) in analogy to general method 113b.

(DMSO-d₆): 10.85 s (1H); 8.68 d (J=8.8 Hz, 1H); 7.97 d (J=9.1 Hz, 1H); 7.92 s (1H); 7.66 d (J=8.0 Hz, 1H); 7.63 d (J=0.8 Hz, 1H); 7.44 dd (J=9,1 Hz/2.8 Hz, 1H); 7.39 d (J=2.8 Hz, 1H); 7.36 d (J=8.0 Hz, 1H); 7.22 d (J=2.0 Hz, 1H); 7.08 dd (J=8.0 Hz/7.0 Hz, 1H); 6.99 dd (J=8.0 Hz/7.0 Hz, 1H); 6.98 s (1H); 6.49 s (1H); 4.93 m (1H); 4.42 m (1H); 3.94 s (3H); 3.86 s (3H); 3.73 s (3H); 3.61 m (2H); 3.05 dd (J=14.9 Hz/6.3 Hz, 1H); 2.93 dd (J=14.9 Hz/7.8 Hz, 1H).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 116 | N-[(R)-1-(Hydroxy- methyl)-2-(1H- indol-3-yl)ethyl]- 6-methoxy-2-(5- methoxybenzofuran-2- yl)quinoline-4-carboxamide; D-Tryptophanol and 6-Methoxy-2-(5- methoxybenzofuran-2- yl)quinoline-4-carboxylic acid | 115 | (DMSO-d₆): 10.85 s(1H); | |
| # 8.71 d(J=8.6 Hz, 1H); 8.02 d(J=9.1 Hz, 1H); 7.94 s(1H); 7.67 d(J=8.0 Hz, 1H); 7.66 d(J=8.6 Hz, 1H); 7.65 s(1H); 7.46 dd(J=9.1 Hz/2.8 Hz, 1H); 7.41 d(J=2.8 Hz, 1H); 7.36 d(J=8.0 Hz, 1H); 7.27 d(J=2.8 Hz, 1H); 7.23 d(J=2.0 Hz, 1H); 7.09 dd(J=8.0 Hz, 1H); 7.09 dd(J=8.0 Hz/7.0 Hz, 1H); 7.02 dd Hz/7.0 Hz, 1H); 7.02 dd (J=8.6 Hz/2.8 Hz, | | | | |
| # 1H); (J=8.6 Hz/2.8 Hz, 1H); 6.99 dd (J=8.0 Hz/7.0 Hz, 1H); 4.93 m(1H); 4.43 m(1H); 3.845(3H); 3.745 (3H); 3.61 m(2H); 3.06 dd (J=14.7 Hz/5.6 Hz, 1H); 2.94 dd(J=14.7 Hz/8.1 Hz, 1H). | 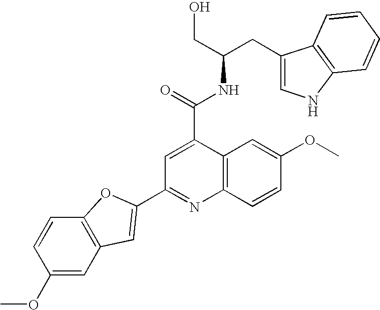 | | | |
| 117 | 2-(7-Ethoxybenzo- furan-2-yl)-N- [(R)-1-(hydroxy- methyl)-2-(1H- indol-3-yl)ethyl]-6- methoxyquinoline-4- carboxamide; D-Tryptophanol and 2-(7-Ethoxybenzo- furan-2-yl)-6- methoxyquinoline-4- carboxylic acid | 115 | (DMSO-d₆): 10.84 s (1H); 8.74 d (J=8.6 Hz, 1H); | |
| # 8.04 d (J=9.1 Hz, 1H); 7.96 s (1H); 7.70 s (1H); 7.68 d (J=8.0 Hz, 1H); 7.47 dd (J=9.1 Hz/2.8 Hz, 1H); 7.42 d (J=2.8 Hz, 1H); 7.36 d (J=8.0 Hz, 1H); 7.32 d (J=7.8 Hz, 1H); 7.24 d (J=2.3 Hz, 1H); 7.23 dd (J=7.8 Hz/7.8 Hz, 1H); 7.08 dd Hz/7.8 Hz, 1H); 7.08 dd (J=8.0 Hz/7.0 Hz, 1H); 7.03 d (J=7.8 Hz, 1H); 7.01 dd (J=8.0 | | | | |
| # Hz/7.0 Hz, 1H); 4.94 m (1H); 4.45 m (1H); 4.30 q (J=6.9 Hz, 2H); 3.73 s (3H); 3.62 m (2H); 3.06 dd (J=14.7 Hz/ 5.6 Hz, 1H); 2.95 dd (J=14.7 Hz/8.3 Hz, 1H); 1.48 t (J=6.9 Hz, 3H). | 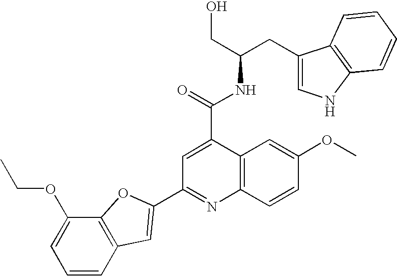 | | | |
| 118 | N-[(R)-1-(Hydroxy- methyl)-2-(1H- indol-3-yl)ethyl]- 6-methoxy-2-(6- methoxybenzofuran-2- yl)quinoline-4-carboxamide; D-Tryptophanol and 6-Methoxy-2-(6- methoxy-2-(6- methoxybenzofuran-2- yl)quinoline-4- carboxylic acid | 115 | (DMSO-d₆): 10.85 s (1H); 8.70 d (J=8.6 Hz, 1H); | |
| # 7.99 d (J=9.1 Hz, 1H); 7.90 s (1H); 7.67 d (J=8.0 Hz, 1H); 7.65 d (J=8.6 Hz, 1H); 7.64 s (1H); 7.45 dd (J=9.1 Hz/2.8 Hz, 1H); 7.41 d (J=2.8 Hz, 1H); 7.36 d (J=8.0 Hz, 1H); 7.27 d (J=2.1 Hz, 1H); 7.23 d (J=2.3 Hz, 1H); 7.09 dd (J=8.0 Hz/7.0 Hz, 1H); 6.99 dd (J=8.0 Hz/7.0 Hz, 1H); 6.97 dd (J=8.6 Hz/ | | | | |
| # 2.3 Hz, 1H); 4.92 m (1H); 4.44 m (1H); 3.87 s (3H); 3.73 s (3H); 3.61 m (2H); 3.06 dd (J=14.4 Hz/5.6 Hz, 1H); 2.98 dd (J=14.4 Hz/8.1 Hz, 1H); | 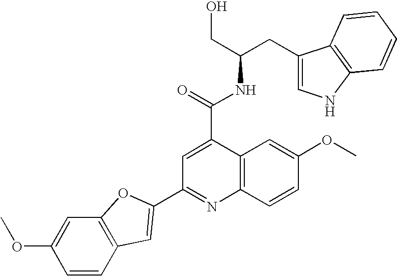 | | | |
| 119 | 2-(7-Fluorobenzo- furan-2-yl)-N- [(R)-1-(hydroxy- methyl)-2-(1H- indol-3-yl)ethyl]-6- methoxyquinoline-4- carboxamide; D-Tryptophanol and 2-(7-Fluorobenzo- furan-2-yl)-6- methoxyquinoline- 4-carboxylic acid | 115 | (DMSO-d₆): 10.85 s (1H); | |
| # 8.75 s (1H); 8.05 d (J=9.1 Hz, 1H); 7.99 s (1H); 7.83 d (J=3.0 Hz, 1H); 7.67 d (J=8.0 Hz, 1H); 7.62 m (1H); 7.49 dd (J=9.1 Hz/2.8 Hz, 1H); 7.43 d (J=2.8 Hz, 1H); 7.36 d (J=8.0 Hz, 1H); 7.34 m (2H); 7.23 d (J=2.0 Hz, 1H); 7.08 dd (J=8.0 Hz/ 7.0 Hz, 1H); 6.99 dd (J=8.0 Hz/7.0 Hz, 1H); 4.95 m (1H); 4.44 m (1H); 3.74 s (3H); 3.62 m (2H); | | | | |
| # dd (J=14.7 Hz/5.6 Hz, 1H); 2.94 dd (J=14.7 Hz/ 8.1 Hz, 1H). | 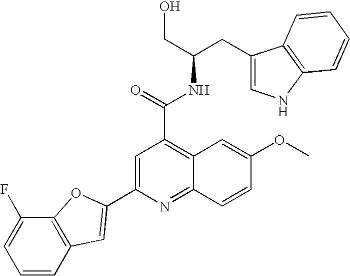 | | | |
| 120 | 2-(4-Fluorobenzo- furan-2-yl)-N- [(R)-1-(hydroxy- methyl)-2-(1H- indol-3-yl)ethyl]-6- methoxyquinoline-4- carboxamide; D-Tryptophanol and 2-(4-Fluorobenzo- furan-2-yl)-6- methoxyquinoline-4- carboxylic acid acid | 115 | (DMSO-d₆): 10.88 s (1H); 8.75 d (J=8.6 Hz, 1H); | |
| # 8.04 d (J=9.1 Hz, 1H); 8.02 s (1H); 7.83 d (J=1.0 Hz, 1H); 7.66 d (J=8.3 Hz, 1H); 7.66 d (J=8.0 Hz, 1H); 7.48 dd (J=9.1 Hz/2.8 Hz, 1H); 7.45 ddd (J=8.3 Hz/8.3 Hz/ 5.8 Hz, 1H); 7.42 d (J=2.8 Hz, 1H); 7.36 d (J=8.0 Hz, 1H); 7.23 d (J=2.0 Hz, 1H); 7.19 dd (J=9.6 Hz/8.3 Hz, 1H); 7.08 dd (J=8.0 Hz/7.0 | | | | |
| # Hz, 1H); 6.98 dd (J=8.0 Hz/ 7.0 Hz, 1H); 4.97 m (1H); 4.43 m (1H); 3.74 s (3H); 3.62 m (2H); 3.06 dd (J=14.7 Hz/5.8 Hz, 1H); 2.94 dd (J=14.7 Hz/8.1 Hz, 1H). | 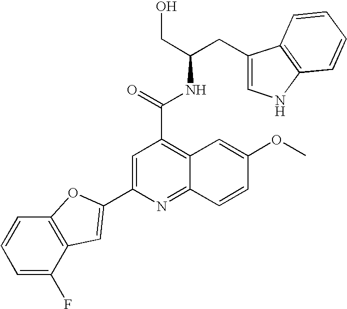 | | | |
| 121 | N-[(R)-1-(Hydroxy- methyl)-2-(1H- indol-3-yl)ethyl]- 6-methoxy-2-(5- methylbenzofuran-2- yl)quinoline-4- carboxamide; D-Tryptophanol and 6-Methoxy-2-(5- methylbenzofuran-2- yl)quinoline-4- carboxylic acid | 115 | (DMSO-d₆): 10.86 s (1H); | |
| # 8.72 d (J=8.6 Hz, 1H); 8.01 d (J=9.1 Hz, 1H); 7.94 s (1H); 7.67 d (J=8.0 Hz, 1H); 7.64 s (1H); 7.63 d (J=8.6 Hz, 1H); 7.56 d (J=1.5 Hz, 1H); 7.46 dd (J=9.1 Hz/2.8 Hz, 1H); 7.41 d (J=2.8 Hz, 1H); 7.36 d (J=8.0 Hz, 1H); 7.24 dd (J=8.6 Hz/1.5 Hz, 1H); 7.23 d (J=2.0 Hz, 1H); 7.09 dd (J=8.0 Hz/7.0 Hz, 1H); | | | | |
| # 6.99 dd (J=8.0 Hz/7.0 Hz, 1H); 4.94 m (1H); 4.43 m (1H); 3.74 s (3H); 3.61 m (2H); 3.06 dd (J=14.7 Hz/ 5.8 Hz, 1H); 2.94 dd (J=14.7 Hz/8.1 Hz, 1H); 2.44 s (3H). | 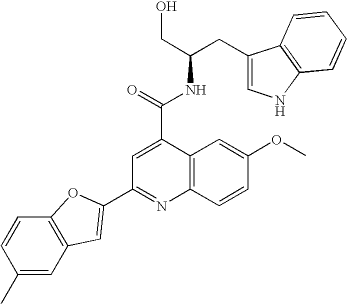 | | | |
| 122 | N-[(R)-1-(Hydroxy- methyl)-2-(1H- indol-3-yl)ethyl]-6- methoxy-2-(7- methylbenzofuran-2- yl)quinoline-4- carboxamide; D-Tryptophanol and 6-Methoxy-2-(7- methylbenzofuran-2- yl)quinoline-4- carboxylic acid | 115 | (DMSO-d₆): 10.86 s (1H); 8.75 d (J=8.6 Hz, 1H); | |
| # 8.03 d (J=9.1 Hz, 1H); 7.98 s (1H); 7.70 s (1H); 7.67 d(J=8.0 Hz, 1H); 7.60 m (1H); 7.47 dd (J=9.1 Hz/2.8 Hz, 1H); 7.43 d (J=2.8 Hz, 1H); 7.36 d (J=8.0 Hz, 1H); 7.24 m (3H); 7.08 dd (J=8.0 Hz/ 7.0 Hz, 1H); 6.99 dd (J=8.0 Hz/7.0 Hz, 1H); 4.95 m (1H); 4.43 m (1H); 3.74 s (3H); 3.62 m (2H); 3.06 dd (J=14.7 Hz/5.6 Hz, 1H); 2.95 dd (J=14.7 | | | | |
| # Hz/ 8.3 Hz, 1H); 2.61 s (3H). | 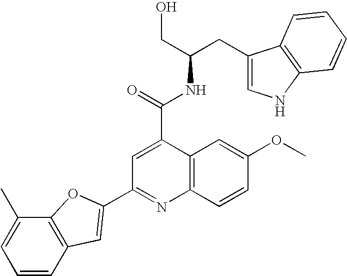 | | | |
| 123 | N-[(R)-1-(Hydroxy- methyl)-2-(1H- indol-3-yl)ethyl]- 6-methoxy-2-(4- methoxybenzofuran-2- yl)quinoline-4- carboxamide; D-Tryptophanol and 6-Methoxy-2-(4- methoxybenzofuran-2- yl)quinoline-4- carboxylic acid | 115 | (DMSO-d₆): 10.85 s (1H); 8.70 d (J=8.6 Hz, 1H); | |
| # 8.01 d (J=9.1 Hz, 1H); 7.99 s (1H); 7.73 s (1H); 7.67 d (J=8.0 Hz, 1H); 7.46 dd (J=9.1 Hz/2.8 Hz, 1H); 7.41 d (J=2.8 Hz, 1H); 7.37 m (2H); 7.36 d (J=8.0 Hz, 1H); 7.22 s (1H); 7.08 dd (J=8.0 Hz/ (1H); 7.08 dd (J=8.0 Hz/ 7.0 Hz, 1H); 6.99 dd (J=8.0 Hz/7.0 Hz, 1H); 6.88 dd (J=7.1 Hz/1.8 Hz, 1H); 4.94 m (1H); 4.43 m (1H); 3.98 s (3H); | | | | |
| # 3.73 s (3H); 3.61 m (2H); 3.05 dd (J=14.7 Hz/5.6 Hz, 1H); 2.93 dd (J=14.7 Hz/8.1 Hz, 1H). | 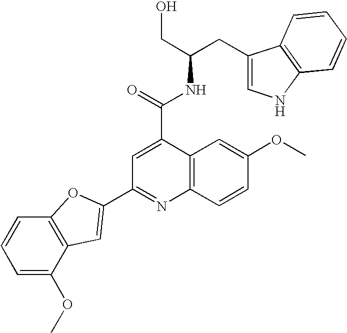 | | | |
| 124 | N-[(R)-1-(Hydroxy- methyl)-2-(1H- indol-3-yl)ethyl]-6- methoxy-2-[5- (trifluoromethoxy)- benzofuran-2- yl]quinoline-4- carboxamide: D-Tryptophanol and 6-Methoxy-2-[5- (trifluoromethoxy)- benzofuran-2- yl]quinoline-4- carboxylic acid | 115 | (DMSO-d₆): 10.86 s (1H); | |
| # 8.75 d (J=8.6 Hz, 1H); 8.04 d (J=9.1 Hz, 1H); 7.98 s (1H); 7.90 d (J=8.8 Hz, 1H); 7.83 d (J=1.5 Hz, 1H); 7.78 d (J=0.8 Hz, 1H); 7.66 d (J=8.0 Hz, 1H); 7.49 dd (J=9.1 Hz/2.8 Hz, 1H); 7.43 dd (J=8.8 Hz/1.5 Hz, 1H); 7.42 d (J=2.8 Hz, 1H); 7.36 d (J=8.0 Hz, 1H); 7.23 d (J=2.0 Hz, 1H); 7.08 dd (J=8.0 | | | | |
| # Hz/ 7.0 Hz, 1H); 6.99 dd (J=8.0 Hz/7.0 Hz, 1H); 4.95 m (1H); 4.43 m (1H); 3.74 s (3H); 3.61 m (2H); 3.06 dd (J=14.7 Hz/5.6 Hz, 1H); 2.94 dd (J=14.7 Hz/ 8.1 Hz, 1H). | 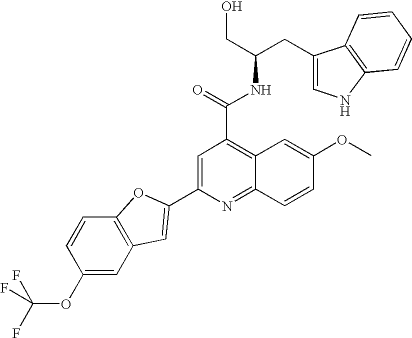 | | | |

### EXAMPLE 125

### 4-Ethoxybiphenyl-3-carboxylic acid [(R)-1-hydroxymethyl-2-(1H-indol-3-yl)ethyl]amide

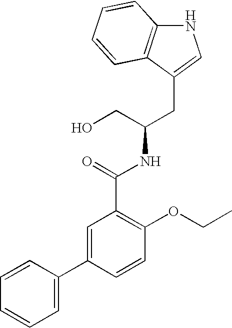

### 125a) Ethyl 5-bromo-2-ethoxybenzoate

5-Bromo-2-hydroxybenzoic acid (5 g) and potassium carbonate (6.37 g) in acetone (230 ml) were stirred under reflux under a nitrogen atmosphere and, at the boiling point, iodoethane (5×5 ml) was slowly added at intervals of 1 hour. Stirring under reflux was continued for 4 hours. The solvent was distilled out in a rotary evaporator, the residue was taken up in ethyl acetate and extracted with water and saturated aqueous NaCl solution, and the combined organic phases were freed of solvent. Flash chromatography resulted in 4.1 g (65% yield) of the title compound. MS (ESI, +): 274 (M+1).

### 125b) 5-Bromo-2-ethoxybenzoic acid

Ethyl 5-bromo-2-ethoxybenzoate (5 g) were stirred under reflux in potassium hydroxide (10% strength in ethanol, 50 ml) for twelve hours. The cooled reaction mixture was mixed with water, and the remaining ethanol was distilled out in a rotary evaporator. The remaining aqueous phase was washed with diethyl ether and acidified by adding 2N HCl. The precipitated reaction product was filtered off and washed with water. Drying in vacuo resulted in 4.25 g (95% yield) of the title compound, which was employed without further purification in the next stage.

MS (ESI, +): 246 (M+1)

### 125c) Methyl (R)-2-(5-bromo-2-ethoxybenzoylamino)-3-(1H-indol-3-yl)-propionate

5-Bromo-2-ethoxybenzoic acid (500 mg), (D)-tryptophan methyl ester hydrochloride (520 mg), EDC (390 mg), HOBt (310 mg) and diisopropylethylamine (0.36 ml) in DMF (10 ml) were stirred together at room temperature overnight. The reaction mixture was concentrated, taken up in ethyl acetate and extracted several times with water. The combined organic phases were freed of solvent, and the reaction mixture was purified by flash chromatography. 660 mg of the title compound (73% yield) were obtained. MS (ESI, +): 446 (M+1)

### 125d) 5-Bromo-2-ethoxy-N-[(R)-1-hydroxymethyl-2-(1H-indol-3-yl)ethyl]benzamide

A solution of methyl (R)-2-(5-bromo-2-ethoxybenzoylamino)-3-(1H-indol-3-yl)-propionate (500 mg) in THF (10 ml) was cooled to −10° C., and a solution of lithium borohydride in THF (0.84 ml, 2 mmol/ml) was slowly added dropwise. The mixture was stirred overnight and then 1N HCl was cautiously added. The solvent was distilled out in a rotary evaporator, and the remaining aqueous phase was extracted with ethyl acetate. The combined organic phases were freed of solvent and dried in vacuo. 435 mg of the title compound (93% yield) were obtained after crystallization from ethanol. MS (ESI, +): 418 (M+1)

### 125e) 4-Ethoxybiphenyl-3-carboxylic acid [(R)-1-hydroxymethyl-2-(1H-indol-3-yl)ethyl]amide

5-Bromo-2-ethoxy-N-[(R)-1-hydroxymethyl-2-(1H-indol-3-yl)ethyl]benzamide (200 mg), phenylboronic acid (64 mg), sodium carbonate (2 M solution in water, 1 ml) and Pd(PPh₃)₄ (6 mg) were heated to reflux together in toluene (6 ml) and ethanol (0.4 ml) overnight. The reaction mixture was filtered and the filtrate was concentrated. The residue was taken up in ethyl acetate and extracted with water. The organic phases were dried and the solvent was distilled off in a rotary evaporator. Flash chromatography resulted in 45 mg of the title compound (21% yield).

(DMSO-d₆): 10.78 s (1H); 8.37 d (J=8 Hz, 1H); 8.13 s (1H); 7.76-7.50 broad m (5H); 7.42 m (2H); 7.29 m (2H); 7.03 m (1H); 6.91 m (1H); 4.30 m (1H); 4.11 m (2H); 3.42 m, (2H): 2.95 m (2H); 1.28 m (3H).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 126 | 4-Ethoxy-3′-fluoro-4′- propoxybiphenyl-3- carboxylic acid [(R)-2-hydroxy-1- (1H-indol-3- ylmethyl)ethyl]amide; 5-Bromo-2-ethoxy-N- [(R)-2-hydroxy-1-(1H- indol-3-ylmethyl)- ethyl]benzamide and 3-Fluoro-4- propoxyphenyl- boronic acid | 125 | | |
| # (DMSO-d₆): 10.78 s (1H); 8.36 d (J=8.1 Hz, 1H); 8.08 s (1H); 7.70 m (2H); 7.47 d (J=12.9 Hz, 1H); 7.37 m (1H); 7.35 m (1H): 7.15 m (4H); 7.03 m (1H); 6.93 m (1H); 4.90 m (1H); 4.22 m (1H); 4.12 m (2H); 3.46 m (1H); 3.40 m (1H); 2.95 m (2H); 1.73 m (2H); 1.27 m (3H); 0.98 m (3H). | 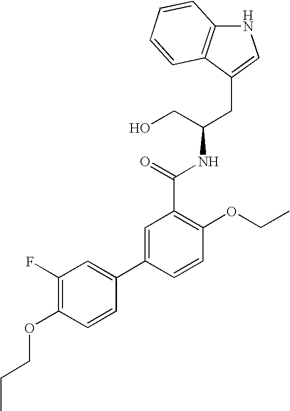 | | | |
| 127 | 2-Ethoxy-N-[(R)-1- hydroxymethyl-2- (1H-indol-3- yl)ethyl]-5-(6-methoxypyridin-3- yl)benzamide; 5-Bromo-2-ethoxy- N-[(R)-2- hydroxy-1- (1H-indol-3- ylmethyl)ethyl]- benzamide and 2-Methoxy-5- pyridineboronic acid | 125 | (DMSO-d₆): 10.79 s (1H); | |
| # 8.40 d (J=2.5 Hz, 1H); 8.36 d (J=8.1 Hz, 1H); 8.07 s (1H); 7.94 dd (J=2.8/8.6 Hz, 1H); 7.68 m (2H); 7.30 d (J=8.1 Hz, 1H); 7.18 d (J=8.6 Hz, 1H); 7.13 s (1H); 7.03 m (1H); 6.93 m (1H); 6.88 m (1H); 4.22 m (1H); 4.12 m (2H); 3.86 s (3H); 3.47 m (2H); 2.95 m (2H); 1.28 m (3H). | 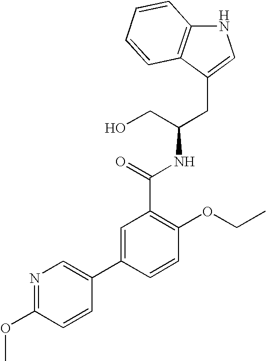 | | | |
| 128 | 4-Ethoxy-2′-fluoro-3′- methoxybiphenyl- 3-carboxylic acid [(R)-2-hydroxy-1- (1H-indol-3- ylmethyl)ethyl]amide; 5-Bromo-2-ethoxy- N-[(R)-2- hydroxy-1-(1H-indol-3- ylmethyl)ethyl]benzamide and 2-Fluoro-3-methoxy- phenyl-boronic acid | 125 | (DMSO-d₆): 10.79 s (1H); | |
| # 8.36 d (J=8.1 Hz, 1H); 8.02 s (1H); 7.65 d (J=8.1 Hz, 1H); 7.60 m (1H); 7.29 d (J=8.1 Hz, 1H); 7.18 m (4H); 7.00 m (2H); 6.93 m (1H); 4.90 m (1H); 4.23 m (1H); 4.13 m (2H); 3.84 s (3H); 3.46 m (1H); 3.38 m (1H); 2.95 m (2H); 1.28 m (3H). | 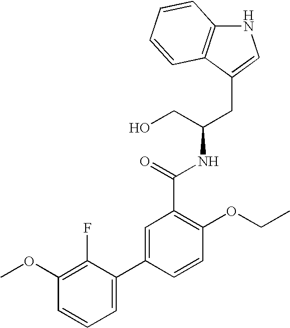 | | | |
| 129 | 4′-Acetylamino-4- ethoxybiphenyl- 3-carboxylic acid [(R)-1-hydroxy- methyl-2- (1H-indol-3-yl)- ethyl]amide; 5-Bromo-2-ethoxy- N-[(R)-2- hydroxy-1-(1H- indol-3-ylmethyl)- ethyl]benzamide and 4-Acetamidophenyl- boronic acid | 125 | (DMSO-d₆): 10.82 s (1H); | |
| # 10.04 s (1H); 8.43 d (J=8.1 Hz, 1H); 8.19 d (J=2.5 Hz, 1H); 7.89 s (1H); 7.73 m (2H); 7.58 m (1H); 7.33 m (3H); 7.26 d (J=8.9 Hz, 1H); 7.18 s (1H); 7.07 m (1H); 6.96 m (1H); 4.29 m (1H); 4.17 m (2H); 3.48 m (2H); 2.99 m (2H); 2.08 s (3H); 1.33 m (3H). | 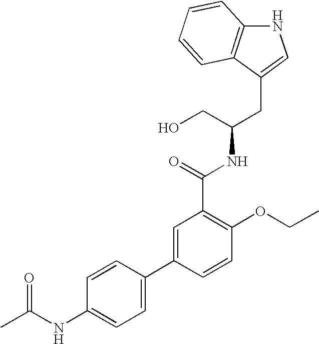 | | | |
| 130 | 2-Ethoxy-N-[(R)-1- hydroxymethyl-2- (1H-indol-3- yl)ethyl]-5-(2- methoxypyrimidin- 5-yl)benzamide; 5-Bromo-2-ethoxy-N- [(R)-2- hydroxy-1- (1H-indol-3- ylmethyl)ethyl]- benzamide and 2-Methoxypyrimidine- 5-boronic acid | 125 | (CDCl₃): 8.88 s (2H); 8.49 | |
| # d (J=7.6 Hz, 1H); 8.41 s (1H); 8.17 s (1H); 7.70 d (J=7.6 Hz, 1H); 7.56 d (J=8.3 Hz, 1H); 7.37 d (J=8.1 Hz, 1H); 7.19 m (1H); 7.11 m (2H); 7.02 d (J=8.3 Hz, 1H); 4.59 m (2H); 4.10 m (5H); 3.84 m (2H); 3.16 m (2H); 1.28 m (3H). 3.16 m (2H); 1.28 m (3H). | 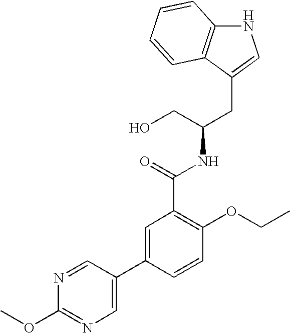 | | | |
| 131 | 4-Ethoxy-5′-fluoro-3- methoxybiphenyl- 3-carboxylic acid [(R)-2-hydroxy-1- (1H-indol-3- ylmethyl)ethyl]amide; 5-Bromo-2-ethoxy-N-[(R)-2- hydroxy-1-(1H-indol-3- ylmethyl)ethyl]- benzamide and 3-Fluoro-5-methoxyphenyl- boronic acid | 125 | (DMSO-d₆): 10.78 s (1H); 8.35 d (J=7.8 Hz, 1H); | |
| # 8.10 s (1H); 7.77 dd (J=2.5 Hz/8.8 Hz, 1H); 7.66 d (J=7.6 Hz, 1H); 7.61-7.49 m (3H); 7.30 d (J=8.1 Hz, 1H); 7.18 d (J=7.8 Hz, 1H); 7.13 s (1H); 7.04-6.91 m (3H); 6.77 m (1H); 4.90 m (1H); 4.21 m (1H); 4.12 m (2H); 3.81 s (1H); 3.42 m (2H); 2.95 m (2H); 1.30 m (3H). | 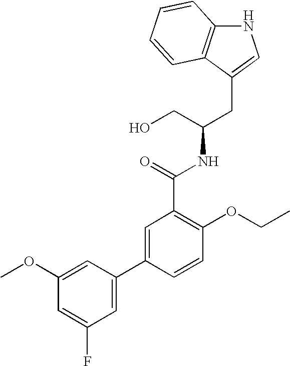 | | | |
| 132 | 4-Ethoxy-3′,4′-difluoro-5′- methoxybiphenyl-3- carboxylic acid [(R)-2-hydroxy-1- (1H-indol-3- ylmethyl)ethyl]amide; 5-Bromo-2-ethoxy-N- [(R)-2-hydroxy-1- (1H-indol-3- ylmethyl)ethyl]- benzamide and 3,4-Difluoro-5-methoxy- phenyl-boronic acid | 125 | (DMSO-d₆): 10.78 s (1H); | |
| # 8.34 d (J=8.1Hz, 1H); 8.11 s (1H); 7.78 dd (J=2.5 Hz/8.6 Hz, 1H); 7.67 d(J=7.8 Hz, 1H); 7.30 d (J=8.1 Hz, 1H); 7.25-7.17 m (3H); 7.13 s (1H); 7.02 m (1H); 6.93 m (1H); 4.22 m (1H); 4.14 m (2H); 3.94 s (3H); 3.47 m (1H); 3.39 m (1H); 2.95 m (2H); 1.29 m (3H). | 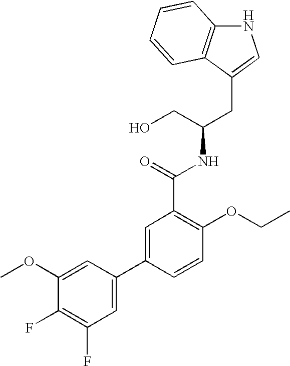 | | | |
| 133 | 4-Ethoxy-4′-fluoro-3′- methoxybiphenyl- 3-carboxylic acid [(R)-2-hydroxy-1- (1H-indol-3- ylmethyl)ethyl]amide; 5-Bromo-2-ethoxy- N-[(R)-2-hydroxy- 1-(1H-indol-3- ylmethyl)ethyl]benzamide and 4-Fluoro-3-methoxy- phenylboronic acid | 125 | (DMSO-d₆): 10.78 s (1H); | |
| # 8.36 d (J=8.1 Hz, 1H); 8.10 s (1H); 7.72 dd (J=2.6 Hz/8.7 Hz, 1H); 7.65 d (J=8.8 Hz, 1H); 7.19 m (6H); 7.02 m (1H); 6.93 m (1H); 4.25 m (1H); 4.14 (m 2H); 3.90 s (3H); 3.45 m (2H); 2.95 m (2H); 1.28 m (3H). | 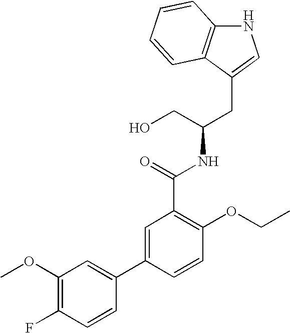 | | | |
| 134 | 3′,5′-Dimethoxy-4- propoxybiphenyl-3- carboxylic acid [(R)-1-hydroxy- methyl-2-(1H-indol- 3-yl)ethyl]amide; N-[(R)-1-Hydroxymethyl-2- (1H-indol-3-yl)ethyl]- 5-iod-2-propoxybenzamide and 3,5-Dimethoxy- phenylboronic acid pinacol ester | 125 | (DMSO-d₆): 10.78 s (1H); | |
| # 8.31 d (J=8.2 Hz, 1H); 8.11 s (1H); 7.71 dd (J=2.6 Hz/8.7 Hz, 1H); 7.65 d (J=7.9 Hz, 1H); 7.30 d (J=8.1 Hz, 1H); 7.15 d (J=8.9 Hz, 1H); 7.12 s (1H); 7.02 m (1H); 6.93 m (1H); 6.70 s (2H); 6.45 s (1H); 4.89 m (1H); 4.24 m (1H); 4.03 m (2H); 3.77 s (6H); 3.46 m (2H); 2.95 m (2H); 1.65 m (2H); 0.91 m (3H). | 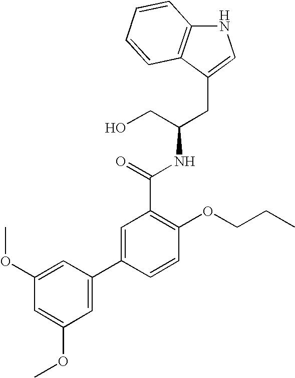 | | | |

### EXAMPLE 135

### 4-Ethoxy-3′-hydroxymethylbiphenyl-3-carboxylic acid [(R)-1-hydroxymethyl-2-(1H-indol-3-yl)ethyl]amide

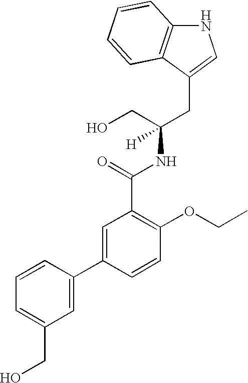

5-Bromo-2-ethoxy-N-[(R)-1-hydroxymethyl-2-(1H-indol-3-yl)ethyl]benzamide (0.2 M solution in THF, 500 μl, prepared by general method 125a-d), triethylamine (0.6 M solution in THF, 200 μl), palladium(II) acetate (0.0375 M in THF, 250 μl), triotolylphosphine (0.05 M solution in THF, 400 μl), 3-hydroxymethyl-phenylboronic acid (0.4 M solution in THF, 200 μl) and water (200 μl) were pipetted into a glass reactor of a microwave and provided with a stirring bar. The mixture was stirred in the microwave at 1200 W, and at 120° C. under pressure for 30 minutes.

The THF was stripped off in a centrifuge, and the residue was then dissolved in 2 ml of DMSO and purified by HPLC.

HPLC-MS: Column Purospher Star RP C18 4.6×125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5°) to 5% (2.5′)

Molecular peak (ESI, M+1): 445.5

Retention time: 8.3 min.

The following compounds were obtained in analogy to the preparation methods described in detail:

| | | Method | | |
|---|---|---|---|---|
| | Product | analogous | HPLC-MS conditions/ | |
| Ex. | reagents | to | ¹H-NMR (400 MHz) δ [ppm] | Structure |
| 136 | 4-Ethoxy-3′- methylsulphanylbiphenyl-3- carboxylic acid [(R)- 2-hydroxy-1-(1H- indol-3-ylmethyl)ethyl]amide; 5-Bromo- 2-ethoxy-N-[(R)-2- hydroxy-1-(1H-indol-3- ylmethyl)ethyl]- benzamide and 3-(Methylthio)phenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 461.6 Retention time: 10.11 min. | 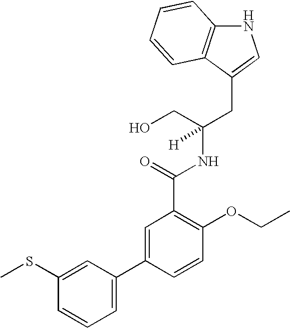 | | | |
| 137 | 3′-Cyano-4-ethoxy- biphenyl-3- carboxylic acid [(R)- 1-hydroxy-methyl- 2-(1H-indol-3- yl)ethyl]amide; 5-Bromo-2-ethoxy-N- [(R)-2-hydroxy-1- (1H-indol-3- ylmethyl)ethyl]- benzamide and 3-Cyanophenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 440.5 Retention time: 9.28 min. | 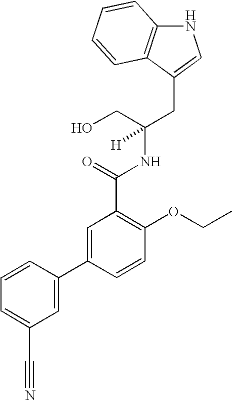 | | | |
| 138 | 2-Ethoxy-5-(6-fluoro-5- methylpyridin-3-yl)- N-[(R)-2-hydroxy- 1-(1H-indol-3- ylmethyl)ethyl]benzamide; 5-Bromo-2-ethoxy- N-[(R)-2-hydroxy- 1-(1H-indol-3- ylmethyl)ethyl]benzamide and 2-Fluoro-3- methylpyridine-5- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 448.5 Retention time: 9.1 min. | 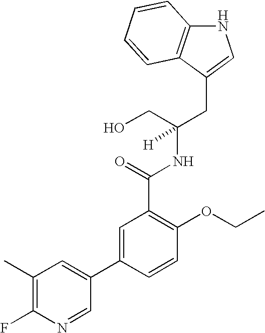 | | | |
| 139 | 4-Ethoxy-4′- trifluoromethoxy- biphenyl-3- carboxylic acid [(R)- 2-hydroxy- 1-(1H-indol-3- ylmethyl)ethyl]amide; 5-Bromo-2-ethoxy- N-[(R)-2- hydroxy-1-(1H- indol-3- ylmethyl)ethyl]benzamide and 4-(Trifluoro- methoxy)phenyl- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 499.5 Retention time: 10.55 min. | 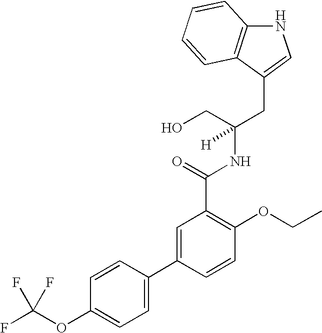 | | | |
| 140 | 5-Benzo[b]thio- phene-3-yl-2- ethoxy-N-[(R)-2- hydroxy-1-(1H-indol-3- ylmethyl)ethyl]benzamide; 5-Bromo-2-ethoxy- N-[(R)-2-hydroxy-1- (1H-indol-3- ylmethyl)ethyl]- benzamide and 1-Benzothiophen-3- ylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 471.6 Retention time: 10.68 min. | 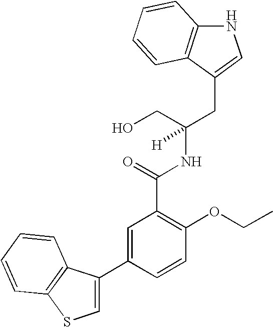 | | | |
| 141 | 4-Ethoxy-2′- trifluoromethyl- biphenyl-3- carboxylic acid [(R)-2-hydroxy- 1-(1H-indol-3- ylmethyl)ethyl]amide; 5-Bromo-2-ethoxy- N-[(R)-2- hydroxy-1-(1H-indol-3- ylmethyl)ethyl]benzamide and 2-(Trifluoromethyl)- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 483.5 Retention time: 10.05 min. | 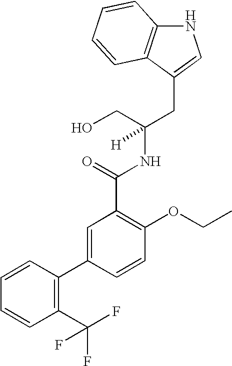 | | | |
| 142 | 4-Ethoxy-2′- trifluoromethoxy- biphenyl-3- carboxylic acid [(R)- 2-hydroxy-1- (1H-indol-3- ylmethyl)ethyl]amide; 5-Bromo-2-ethoxy- N-[(R)-2- hydroxy-1-(1H-indol-3- ylmethyl)ethyl]benzamide and 2-(Trifluoro- methoxy)phenyl- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 499.5 Retention time: 10.25 min. | 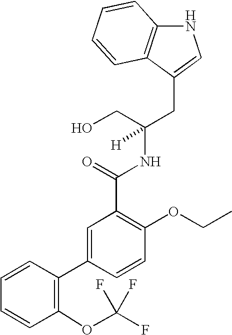 | | | |
| 143 | 4-Ethoxy-3′- trifluoromethoxy- biphenyl-3- carboxy acid [(R)-2-hydroxy- 1-(1H-indol-3- ylmethyl)ethyl]amide; 5-Bromo-2-ethoxy-N- [(R)-2-hydroxy-1-(1H-indol-3- ylmethyl)ethyl]benzamide and 3-(Trifluoromethoxy)- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 499.5 Retention time: 10.52 min. | 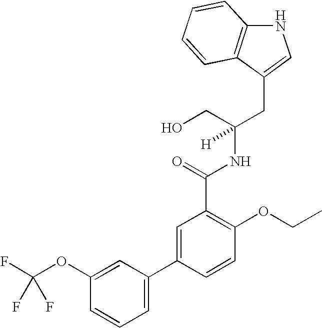 | | | |
| 144 | 4-Ethoxy-3′-fluoro-biphenyl-3- carboxylic acid [(R)- 2-hydroxy- 1-(1H-indol-3- ylmethyl)ethyl]amide; 5-Bromo-2-ethoxy- N-[(R)-2-hydroxy-1- (1H-indol-3- ylmethyl)ethyl]benzamide and 3-Fluorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 433.5 Retention time: 9.8 min. | 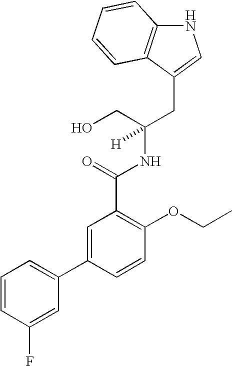 | | | |
| 145 | 4′-Chloro-4-ethoxybiphenyl-3- carboxylic acid [(R)- 2-hydroxy-1-(1H- indol-3-ylmethyl)ethyl]amide; 5-Bromo-2-ethoxy-N- [(R)-2-hydroxy-1-(1H-indol-3- ylmethyl)ethyl]benzamide and 4-Chloro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 449.9 Retention time: 10.32 min. | 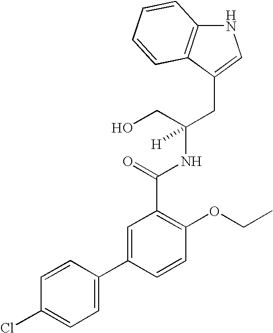 | | | |
| 146 | 4-Ethoxy-4′- methylsulphanylbiphenyl-3- carboxylic acid [(R)-2- hydroxy-1-(1H-indol-3- ylmethyl)ethyl]amide; 5-Bromo-2-ethoxy- N-[(R)-2-hydroxy-1- (1H-indol-3- ylmethyl)ethyl]benzamide and 4-(Methylthio) phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 461.6 Retention time: 10.17 min. | 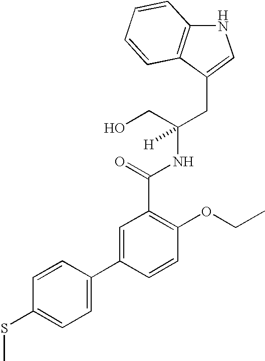 | | | |
| 147 | 4-Ethoxy-3′- trifluoromethylbiphenyl-3- carboxylic acid [(R)-2- hydroxy-1-(1H-indol-3- ylmethyl)ethyl]amide; 5-Bromo-2-ethoxy-N- [(R)-2-hydroxy-1-(1H-indol-3- ylmethyl)ethyl]benzamide and 3-(Trifluoromethyl)- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 483.5 Retention time: 10.35 min. | 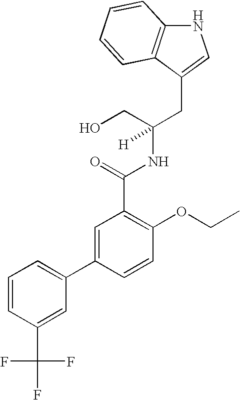 | | | |
| 148 | 3′-Chloro-4-ethoxybiphenyl-3- carboxylic acid [(R)-2- hydroxy-1-(1H-indol-3- ylmethyl)ethyl]amide; 5-Bromo-2-ethoxy-N-[(R)-2- hydroxy-1-(1H-indol-3- ylmethyl)ethyl]benzamide and 3-Chloro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 449.9 Retention time: 10.3 min. | 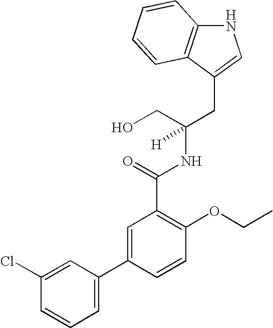 | | | |
| 149 | 4-Ethoxy-3′-methylbiphenyl-3- carboxylic acid [(R)-1-hydroxy- methyl-2-(1H-indol-3- yl)ethyl]amide; 5-Bromo-2-ethoxy-N-[(R)-2- hydroxy-1-(1H-indol-3- ylmethyl)ethyl]benzamide and 3-Methyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 429,5 Retention time: 10,17 min. | 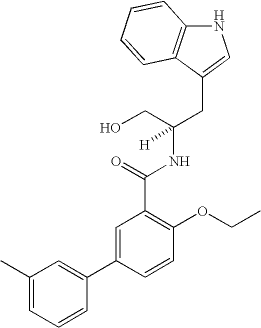 | | | |
| 150 | 5-Benzofuran-2-yl-2-ethoxy-N- [(R)-1-hydroxymethyl-2-(1H- indol-3-yl)ethyl]benzamide; 5-Bromo-2-ethoxy-N-[(R)-2- hydroxy-1-(1H-indol-3- ylmethyl)ethyl]benzamide and Benzo[b]furan- 2-brornic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 455.5 Retention time: 10.61 min. | 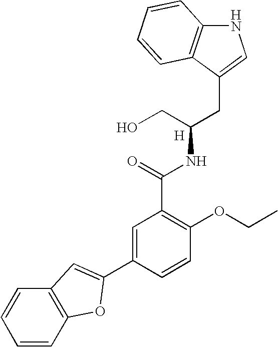 | | | |
| 151 | 4-Ethoxy-2′- methylsulphanylbiphenyl-3- carboxylic acid [(R)-2-hydroxy- 1-(1H-indol-3- ylmethyl)ethyl]amide; 5-Bromo-2-ethoxy-N-[(R)-2- hydroxy-1-(1H-indol-3- ylmethyl)ethyl]benzamide and 2-(Methyl- thio)phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 461.6 Retention time: 10.17 min. | 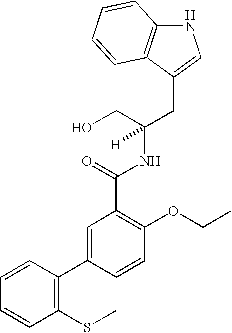 | | | |
| 152 | 2-Ethoxy-N-[(R)-1- hydroxymethyl-2-(1H-indol-3- yl)ethyl]-5-(1H-indol-4- yl)benzamide; 5-Bromo-2-ethoxy-N-[(R)-2- hydroxy-1-(1H-indol-3- ylmethyl)ethyl]benzamide and 1H-indole-4-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 454.5 Retention time: 9.11 min. | 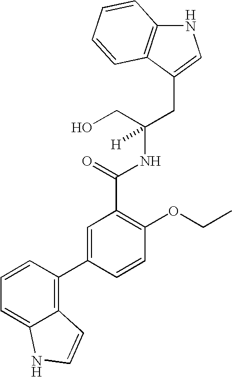 | | | |
| 153 | 2-Ethoxy-N-[(R)-2-hydroxy-1- (1H-indol-3- ylmethyl)ethyl]-5-(4- methylthiophene-2- yl)benzamide; 5-Bromo-2-ethoxy- N-[(R)-2-hydroxy- 1-(1H-indol-3- ylmethyl)ethyl]benzamide and 4-Methyl- thiophene-2-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 435.6 Retention time: 10.07 min. | 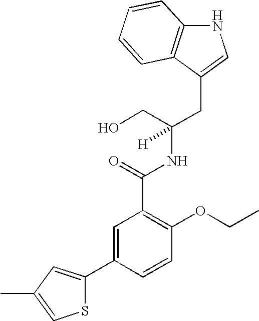 | | | |
| 154 | 3′-Acetylamino-4- ethoxybiphenyl-3- carboxylic acid [(R)-1- hydroxymethyl-2- (1H-indol-3-yl)ethyl]amide; 5-Bromo-2-ethoxy-N-[(R)-2- hydroxy-1-(1H-indol-3- ylmethyl)ethyl]benzamide and 3-Acetamidophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 472.6 Retention time: 8.3 min. | 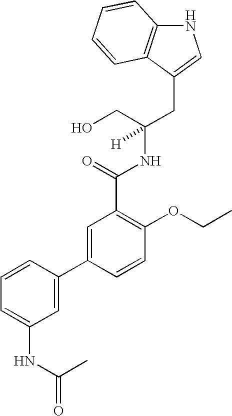 | | | |
| 155 | 4-Ethoxy-2′-methylbiphenyl-3- carboxylic acid [(R)-1- hydroxy-methyl-2- (1H-indol-3- yl)ethyl]amide; 5-Bromo-2-ethoxy-N- [(R)-2- hydroxy-1-(1H-indol-3- ylmethyl)ethyl]benzamide and 2-Methyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 429.5 Retention time: 10.35 min. | 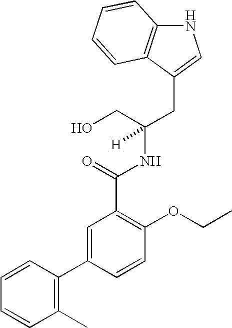 | | | |
| 156 | 2-Ethoxy-N-[(R)-1- hydroxymethyl-2-(1H-indol-3- yl)ethyl]-5-(5-methyl- furna-2-yl)benzamide; 5-Bromo-2-ethoxy-N- [(R)-2-hydroxy-1-(1H- indol-3-ylmethyl)- ethyl]benzamide and 5-Methylfuran-2- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 419.5 Retention time: 9.08 min. | 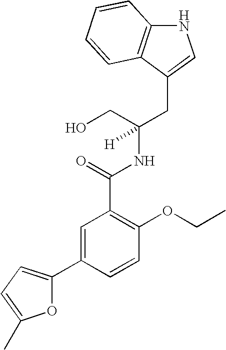 | | | |
| 157 | 3′-Chloro-4-ethoxy-4′- methylbiphenyl-3- carboxylic acid [(R)- 2-hydroxy-1-(1H- indol-3-ylmethyl)- ethyl]amide; 5-Bromo-2-ethoxy-N- [(R)-2-hydroxy-1- (1H-indole-3- ylmethyl)ethyl]- benzamide and 3-Chloro-4-methyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 464.0 Retention time: 10.83 min. | 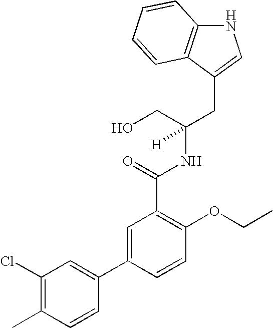 | | | |
| 158 | 5-(2-Chloro-6-methylpyridin-3- yl)-2-ethoxy-N-[(R)-2- hydroxy-1-(1H-indol-3- ylmethyl)ethyl]benzamide; 5-Bromo-2-ethoxy-N- [(R)-2-hydroxy-1- (1H-indol-3- ylmethyl)ethyl]benzamide and 2-Chloro- 6-methylpyridine-3- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 465.0 Retention time: 9.33 min. | 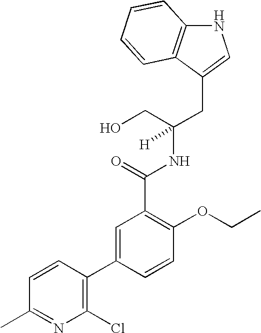 | | | |
| 159 | 4-Ethoxy-4′-fluoro-biphenyl-3- carboxylic acid [(R)- 2-hydroxy-1-(1H-indol-3- ylmethyl)ethyl]amide; 5-Bromo-2-ethoxy-N- [(R)-2-hydroxy-1- (1H-indol-3-ylmethyl)- ethyl]benzamide and 4-Fluorophenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 433.5 Retention time: 9.73 min. | 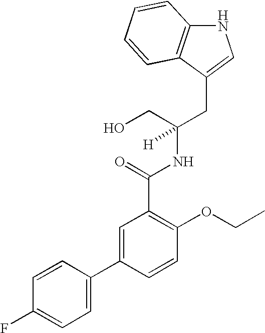 | | | |
| 160 | 2-Ethoxy-N-[(R)-1- hydroxymethyl-2-(1H-indol-3- yl)ethyl]-5-naphthalen-1- ylbenzamide; 5-Bromo-2-ethoxy-N- [(R)-2-hydroxy-1- (1H-indol-3- ylmethyl)ethyl]benzamide and 1-Naphthalene- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 465.6 Retention time: 10.55 min. | 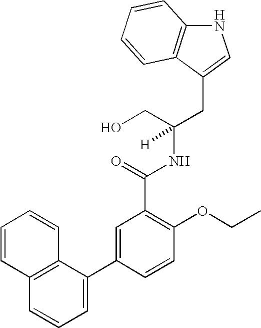 | | | |
| 161 | 5-Benzo[b]thiophene-2-yl-2- ethoxy-N-[(R)-2-hydroxy- 1-(1H-indol-3- ylmethyl)ethyl]benzamide; 5-Bromo-2-ethoxy-N- [(R)-2-hydroxy- 1-(1H-indol-3- ylmethyl)ethyl]benzamide and Benzo[b]thio- phene-2-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 471.6 Retention time: 10.92 min. | 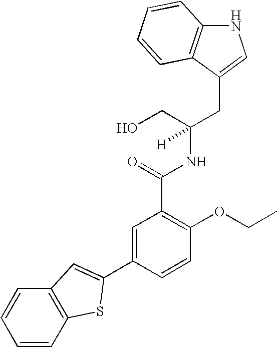 | | | |
| 162 | 4-Ethoxy-4′-methylbiphenyl-3- carboxylic acid [(R)- 1-hydroxy-methyl-2- (1H-indol-3-yl)ethyl]amide; 5-Bromo-2-ethoxy-N- [(R)-2-hydroxy-1- (1H-indol-3- ylmethyl)ethyl]benzamide and 4-Methylphenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 429.5 Retention time: 10.14 min. | 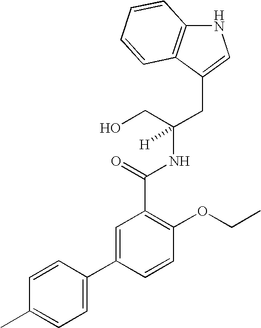 | | | |
| 163 | 2-Ethoxy-N-[(R)-2-hydroxy-1- (1H-indol-3-ylmethyl)ethyl]-5- thiophene-3-ylbenzamide; 5-Bromo-2-ethoxy-N-[(R)-2- hydroxy-1-(1H-indol-3- ylmethyl)ethyl]benzamide and Thiophene-3-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 421.5 Retention time: 9.72 min. | 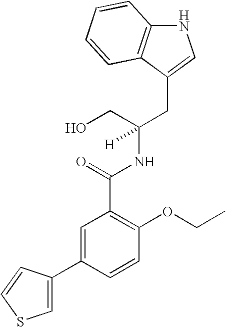 | | | |
| 164 | 4-Ethoxy-4′-methoxybiphenyl-3- carboxylic acid [(R)- 1-hydroxy-methyl-2- (1H-indol-3- yl)ethyl]amide; 5-Bromo-2-ethoxy-N-[(R)-2- hydroxy-1-(1H-indol-3- ylmethyl)ethyl]benzamide and 4-Methoxy- phenylboronic acid | 135 | HPLC-MS: Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 445.5 Retention time: 9.61 min. | 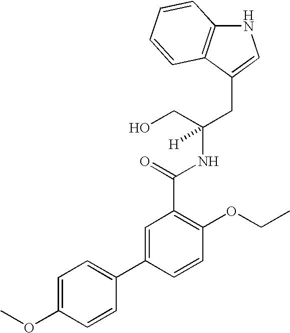 | | | |
| 165 | 2′,4′-Dichloro-4-ethoxybiphenyl- 3-carboxylic acid [(R)-2- hydroxy-1-(1H-indol-3- ylmethyl)ethyl]amide; 5-Bromo-2-ethoxy-N-[(R)-2- hydroxy-1-(1H-indol-3- ylmethyl)ethyl]benzamide and 2,4-Dichloro- phenylboronic acid | 135 | HPLC-MS: Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 484.4 Retention time: 10.78 min. | 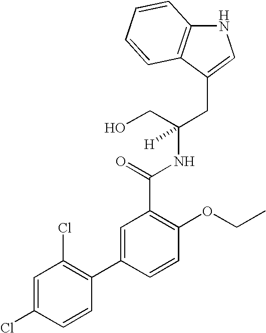 | | | |
| 166 | 4′-Methoxy-4-propoxybiphenyl- 3-carboxylic acid [(R)-1- hydroxymethyl-2-(1H- indol-3-yl)ethyl]amide; N-[(R)-1-Hydroxy- methyl-2-(1H- indol-3-yl)ethyl]- 5-iod-2-propoxy- benzamide and 4-Methoxyphenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 459.6 Retention time: 9.67 min. | 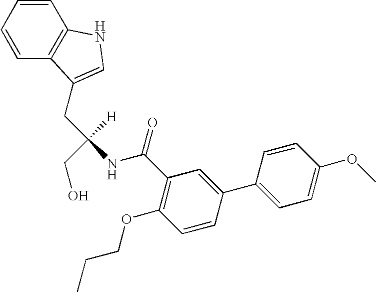 | | | |
| 167 | 4-Propoxybiphenyl-3-carboxylic acid [(R)-1-hydroxymethyl-2- (1H-indol-3-yl)ethyl]amide; N-[(R)-1-Hydroxy- methyl-2-(1H- indol-3-yl)ethyl]-5- iodo-2-propoxy- benzamide and Phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 429.5 Retention time: 9.75 min. | 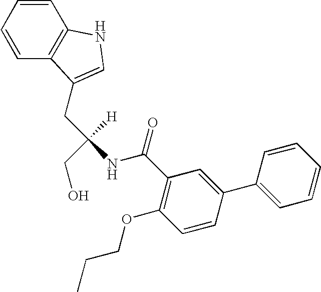 | | | |
| 168 | 5-Benzofuran-2-yl-N-[(R)-1- hydroxymethyl-2-(1H- indol-3- yl)ethyl]-2-propoxy- benzamide; N-[(R)-1-Hydroxy- methyl-2-(1H- indol-3-yl)ethyl]-5-iodo-2- propoxybenzamide and Benzo[b]furan-2- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 469.6 Retention time: 10.52 min. | 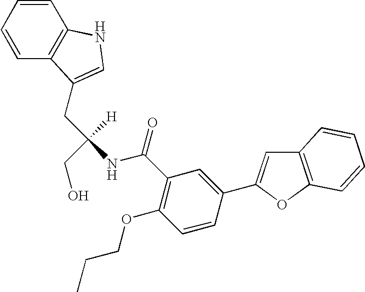 | | | |
| 169 | 3′-Chloro-4-propoxybiphenyl-3- carboxylic acid [(R)-2- hydroxy-1-(1H- indol-3-ylmethyl)ethyl]amide; N-[(R)-1-Hydroxymethyl- 2-(1H-indol-3-yl)ethyl]- 5-iodo-2-propoxybenzamide and 3-Chloro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 464 Retention time: 10.38 min. | 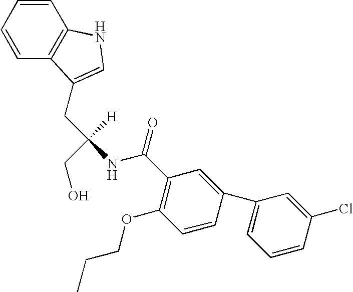 | | | |
| 170 | 5-Benzo[b]thiophen-2-yl-N-[(R)- 2-hydroxy-1-(1H-indol-3- ylmethyl)ethyl]-2- propoxybenzamide; and Benzo[b]thio- phene-2-Boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 485.6 Retention time: 10.84 min. | 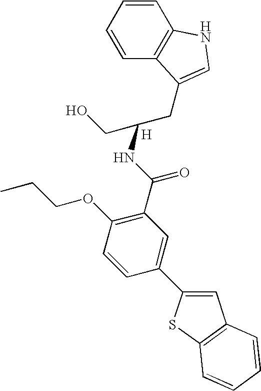 | | | |
| 171 | 3′-Fluoro-4′-methyl-4- propoxybiphenyl-3-carboxylic acid [(R)-2-hydroxy-1- (1H-indol-3- ylmethyl)ethyl]amide; N-[(R)-1-Hydroxy- methyl-2-(1H-indol- 3-yl)ethyl]-5-iodo-2- propoxybenzamide and 3-Fluoro-4-methyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 461.6 Retention time: 10.37 min. | 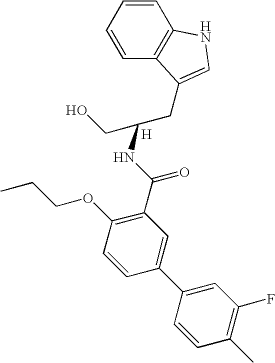 | | | |
| 172 | 4-Propoxy-3′- trifluoromethylbiphenyl-3- carboxylic acid [(R)-2- hydroxy-1-(1H-indol-3- ylmethyl)ethyl]amide; N-[(R)-1-Hydroxy- methyl-2-(1H- indol-3-yl)ethyl]-5- iodo-2-propoxybenzamide and 3-(Trifluoromethyl)- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 497.5 Retention time: 10.41 min. | 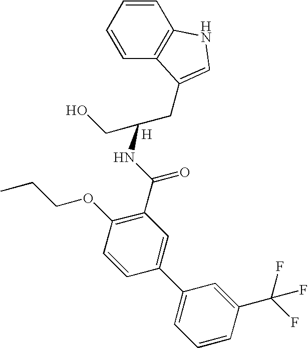 | | | |
| 173 | 2′-Fluoro-5′-methoxy-4- propoxybiphenyl-3-carboxylic acid [(R)-2-hydroxy-1-(1H- indol-3-ylmethyl)ethyl]amide; N-[(R)-1-Hydroxymethyl- 2-(1H-indol-3-yl)ethyl]- 5-iod-2-propoxybenzamide and 2-Fluoro-5-methoxyphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 477.6 Retention time: 9.79 min. | 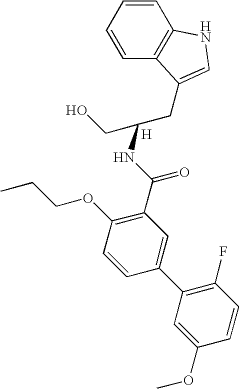 | | | |
| 174 | 4-Propoxy-3′,5′-bis- trifluoromethylbiphenyl-3- carboxylic acid [(R)-2- hydroxy-1-(1H-indol-3- ylmethyl)ethyl]amide; N-[(R)-1-Hydroxymethyl- 2-(1H-indol-3- yl)ethyl]-5-iod-2- propoxybenzamide and 3,5-Bis-(Trifluoromethyl)- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 565.5 Retention time: 11.01 min. | 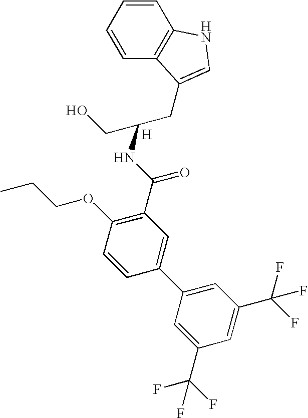 | | | |
| 175 | 4′-Chloro-4-propoxybiphenyl-3- carboxylic acid [(R)-2- hydroxy-1-(1H-indol-3- ylmethyl)ethyl]amide; N-[(R)-1-Hydroxymethyl-2-(1H- indol-3-yl)ethyl]-5-iod-2- propoxybenzamide and 4-Chlorophenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 464 Retention time: 10.44 min. | 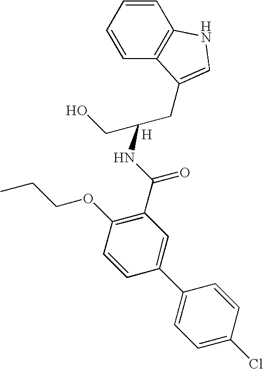 | | | |
| 176 | 5-Benzo[b]thiophene-3-yl-N- [(R)-2-hydroxy-1-(1H-indol-3- ylmethyl)ethyl]-2- propoxybenzamide; N-[(R)-1-Hydroxymethyl-2-(1H- indol-3-yl)ethyl]-5-iodo-2- propoxybenzamide and 1-Benzothiophene-3- yl-Boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 485.6 Retention time: 10.56 min. | 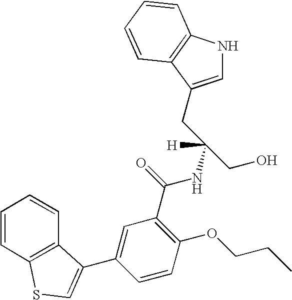 | | | |
| 177 | 4-Propoxy-4′- trifluoromethylbiphenyl-3- carboxylic acid [(R)-2- hydroxy-1-(1H-indol-3- ylmethyl)ethyl]amide; N-[(R)-1-Hydroxymethyl-2-(1H- indol-3-yl)ethyl]-5-iodo-2- propoxybenzamide and 4-Trifluoromethyl)- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 497.5 Retention time: 10.42 min. | 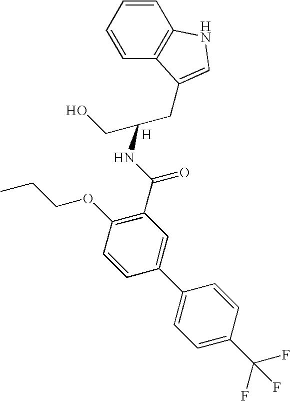 | | | |
| 178 | 3′-Hydroxy-4-propoxybiphenyl- 3-carboxylic acid [(R)-1- hydroxymethyl-2-(1H-indol-3- yl)ethyl]amide; N-[(R)-1-Hydroxymethyl- 2-(1H-indol-3-yl)ethyl]- 5-iodo-2- propoxybenzamide and 3-Hydroxyphenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 445.5 Retention time: 8.56 min. | 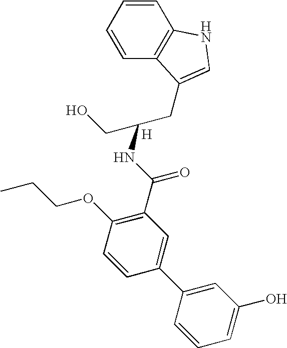 | | | |
| 179 | N-[(R)-1-Hydroxymethyl-2-(1H- indol-3-yl)ethyl]-2-propoxy-5- quinoline-6-ylbenzamide; N-[(R)-1-Hydroxymethyl-2- (1H-indol-3-yl)ethyl]- 5-iodo-2-propoxy- benzamide and Quinoline-6-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 480.6 Retention time: 6.78 min. | 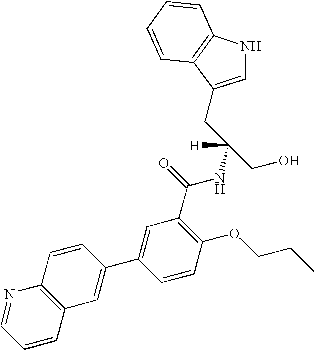 | | | |
| 180 | 5-(6-Fluoro-5-methylpyridin-3- yl)-N-[(R)-2-hydroxy-1-(1H- indol-3-ylmethyl)ethyl]-2- propoxybenzamide; N-[(R)-1-Hydroxymethyl-2-(1H- indol-3-yl)ethyl]-5-iodo-2- propoxybenzamide and 2-Fluoro-3-methylpyridine-5- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 462.5 Retention time: 9.2 min. | 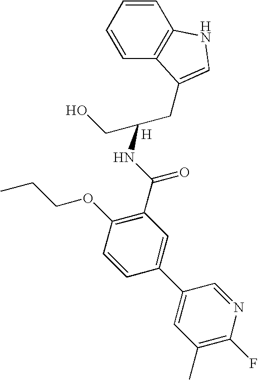 | | | |
| 181 | N-[(R)-1-Hydroxymethyl-2-(1H- indol-3-yl)ethyl]-5-(6- methoxypyridine-3-yl)-2- propoxybenzamide; N-[(R)-1-Hydroxymethyl-2-(1H- indol-3-yl)ethyl]-5-iodo-2- propoxybenzamide and 2-Methoxypyridine-5- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 460.6 Retention time: 8.57 min. | 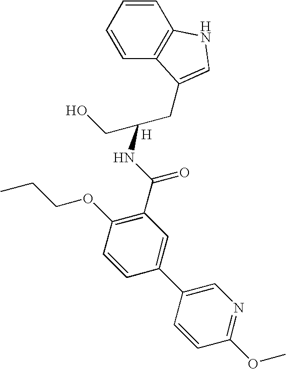 | | | |
| 182 | 3′-Chloro-4′-methyl-4- propoxybiphenyl-3-carboxylic acid [(R)-2-hydroxy-1-(1H-indol- 3-ylmethyl)ethyl]amide; N-[(R)-1-Hydroxymethyl-2-(1H- indol-3-yl)ethyl]-5-iodo-2- propoxybenzamide and 3-Chloro-4-methylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 478 Retention time: 10.94 min. | 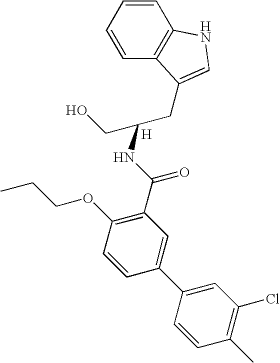 | | | |
| 183 | N-[(R)-1-Hydroxymethyl-2-(1H- indol-3-yl)ethyl]-2-propoxy- 5-pyridine-4-ylbenzamide; N-[(R)-1-Hydroxymethyl- 2-(1H-indol-3-yl)ethyl]- 5-iodo-2-propoxybenzamide and Pyridine-4-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 430.5 Retention time: 6.14 min. | 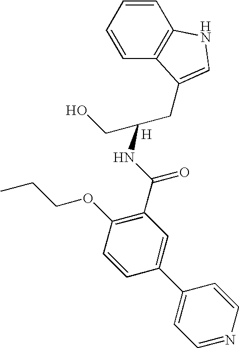 | | | |
| 184 | 3′-Chloro-4′-fluoro-4- propoxybiphenyl-3-carboxylic acid [(R)-2-hydroxy-1-(1H- indol-3-ylmethyl)ethyl]amide; N-[(R)-1-Hydroxymethyl-2- (1H-indol-3-yl)ethyl]-5-iodo-2- propoxybenzamide and 3-Chloro-4-fluorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 482 Retention time: 10.41 min. | 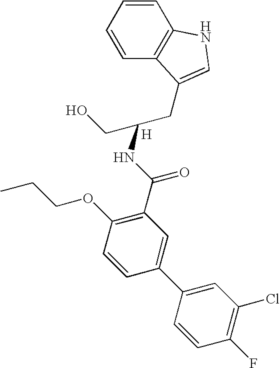 | | | |
| 185 | 3′-Acetylamino-4- propoxybiphenyl-3-carboxylic acid [(R)-1-hydroxymethyl-2- (1H-indol-3-yl)ethyl]amide; N-[(R)-1-Hydroxymethyl-2-(1H- indol-3-yl)ethyl]-5-iodo-2- propoxybenzamide and 3-Acetamidophenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 486.6 Retention time: 8.32 min. | 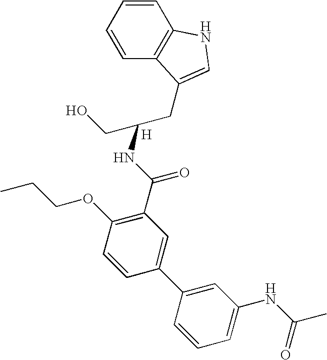 | | | |
| 186 | 3′,4′-Difluoro-4- propoxybiphenyl- 3-carboxylic acid [(R)-2- hydroxy-1-(1H-indol-3- ylmethyl)ethyl]amide; N-[(R)-1-Hydroxymethyl-2- (1H-indol-3-yl)ethyl]-5- iodo-2-propoxybenzamide and 3,4-Difluorophenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular weight, calc. 464.5 Molecular peak (ESI, M + 1): 465.5 Retention time: 9.97 min. | 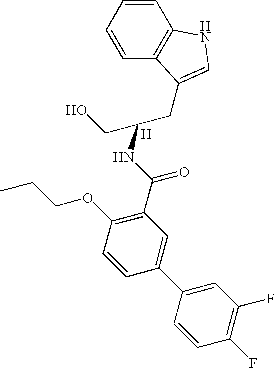 | | | |
| 187 | 3′,5′-Difluoro-4- propoxybiphenyl- 3-carboxylic acid [(R)-2- hydroxy-1-(1H-indol-3- ylmethyl)ethyl]amide; N-[(R)-1-Hydroxymethyl-2- (1H-indol-3-yl)ethyl]- 5-iodo-2-propoxy- benzamide and 3,5-Difluorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 465.5 Retention time: 10.07 min. | 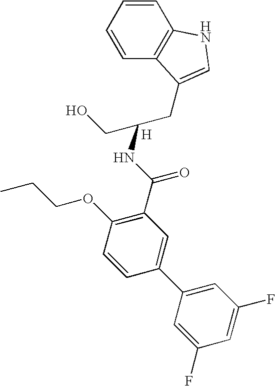 | | | |
| 188 | 3′-Cyano-4-propoxybiphenyl-3- carboxylic acid [(R)-1- hydroxymethyl-2-(1H-indol-3- yl)ethyl]amide; N-[(R)-1-Hydroxymethyl-2- (1H-indol-3-yl)ethyl]-5- iodo-2-propoxybenzamide and 3-Cyanophenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 454.6 Retention time: 9.42 min. | 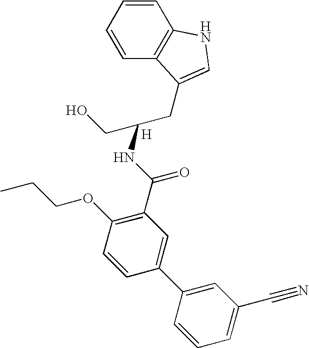 | | | |
| 189 | 5-(2,4-Dimethoxy- pyrimidin-5-yl)- N-[(R)-1-hydroxymethyl-2-(1H- indol-3-yl)ethyl]-2- propoxybenzamide; N-[(R)-1-Hydroxymethyl- 2-(1H-indol-3-yl)ethyl]- 5-iodo-2-indol-3-yl)ethyl]- 5-iodo-2-propoxy- benzamide and 2,4-Dimethoxy- pyrimidine-5- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 491.6 Retention time: 7.16 min. | 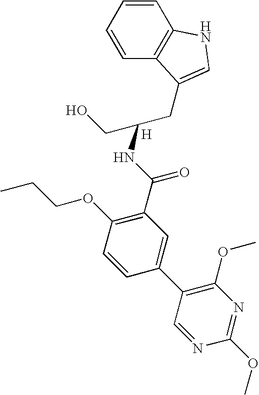 | | | |
| 190 | 2′,3′-Difluoro-4- propoxybiphenyl- 3-carboxylic acid [(R)-2- hydroxy-1-(1H-indol-3- ylmeyhyl)ethyl]amide; N-[(R)-1-Hydroxymethyl-2-(1H- indol-3-yl)ethyl]-5-iodo-2- propoxybenzamide and 2,3-Difluorophenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 465.5 Retention time: 9.88 min. | 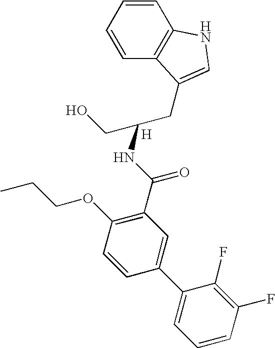 | | | |
| 191 | 2′,5′-Difluoro-4- propoxybiphenyl- 3-carboxylic acid [(R)-2- hydroxy-1-(1H-indol-3- ylmethyl)ethyl]amide; N-[(R)-1-Hydroxy- methyl-2-(1H- indol-3-yl)ethyl]-5-iodo-2- propoxybenzamide and 2,5-Difluorophenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 465.5 Retention time: 9.82 min. | 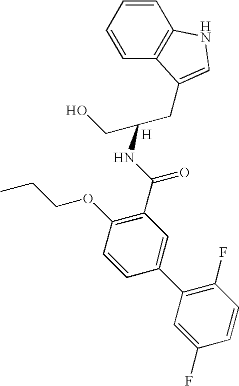 | | | |
| 192 | 5-[(E)-2-(4-Fluoro- phenyl)-vinyl]- N-[(R)-2-hydroxy-1-(1H-indol-3- ylmethyl)ethyl]-2- propoxybenzamide; N-[(R)-1-Hydroxymethyl-2-(1H- indol-3-yl)ethyl]-5-iodo-2- propoxybenzamide and (E)-2-(4-Fluorphenyl)vinyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 473.6 Retention time: 10.31 min. | 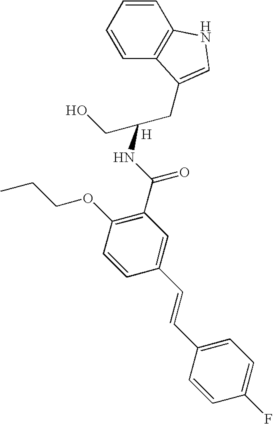 | | | |
| 193 | 5-(5-Cyanothiophen-2-yl)-N- [(R)-2-hydroxy-1-(1H-indol-3- ylmethyl)ethyl]-2- propoxybenzamide; N-[(R)-1-Hydroxymethyl-2- (1H-indol-3-yl)ethyl]-5- iodo-2-propoxybenzamide and 5-Cyanothiophene-2- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 460.6 Retention time: 9.48 min. | 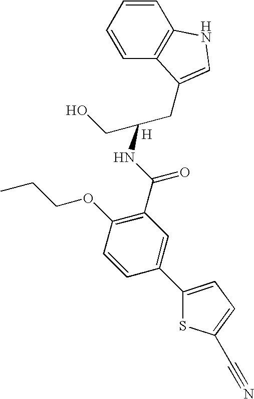 | | | |
| 194 | 2′-Fluoro-3′-methoxy-4- propoxybiphenyl-3-carboxylic acid [(R)-2-hydroxy-1-(1H-indol- 3-ylmethyl)ethyl]amide; N-[(R)-1-Hydroxymethyl-2- (1H-indol-3-yl)ethyl]- 5-iodo-2-propoxybenzamide and 2-Fluoro-3-methoxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 477.6 Retention time: 9.59 min. | 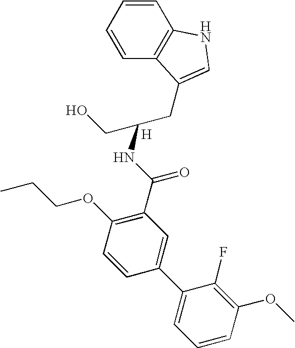 | | | |
| 195 | N-[(R)-1-Hydroxymethyl-2-(1H- indol-3-yl)ethyl]-5-(2- methoxypyrimidine-5-yl)-2- propoxybenzamide; N-[(R)-1-Hydroxymethyl- 2-(1H-indol-3-yl)ethyl]- 5-iodo-2-propoxybenzamide and 2-Methoxypyrimidine- 5-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 461.5 Retention time: 8.09 min. | 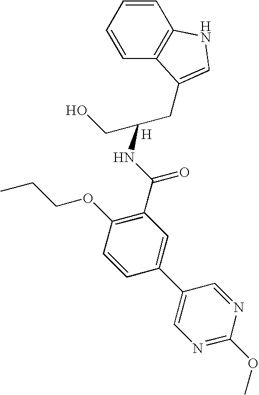 | | | |
| 196 | 4′-Chloro-2′,6′-difluoro-4- propoxybiphenyl-3-carboxylic acid [(R)-2-hydroxy-1-(1H-indol- 3-ylmethyl)ethyl]amide; N-[(R)-1-Hydroxymethyl-2- (1H-indol-3-yl)ethyl]-5- iodo-2-propoxybenzamide and 4-Chloro-2,6-difluoro-phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 500 Retention time: 10.43 min. | 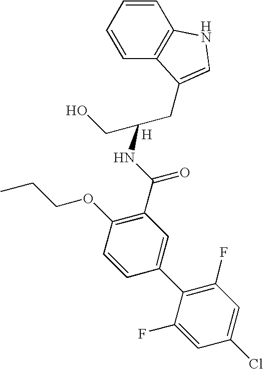 | | | |
| 197 | 3′,5′-Dimethyl-4- propoxybiphenyl-3-carboxylic acid [(R)-1-hydroxymethyl-2- (1H-indol-3-yl)ethyl]amide; N-[(R)-1-Hydroxymethyl-2-(1H- indol-3-yl)ethyl]-5-iodo-2- propoxybenzamide and 3,5-Dimethylphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 457.6 Retention time: 10.71 min. | 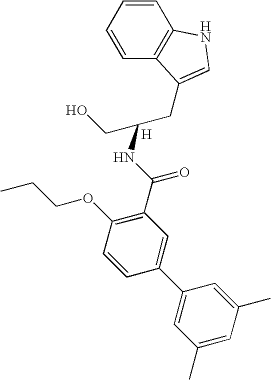 | | | |
| 198 | N-[(R)-1-Hydroxymethyl-2-(1H- indol-3-yl)ethyl]-2-propoxy-5- quinoline-3-ylbenzamide; N-[(R)-1-Hydroxymethyl-2- (1H-indol-3-yl)ethyl]-5- iodo-2-propoxybenzamide and 3-Quinolineboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 480.6 Retention time: 7.07 min. | 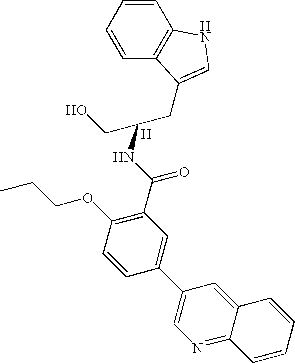 | | | |
| 199 | 4′-Acetylamino-4- propoxybiphenyl-3-carboxylic acid [(R)-1-hydroxymethyl-2- (1H-indol-3-yl)ethyl]amide; N0[(R)-1-Hydroxymethyl-2-(1H- indol-3-yl)ethyl]-5-iodo-2- propoxybenzamide and 4-Acetamidophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 486.6 Retention time: 8.12 min. | 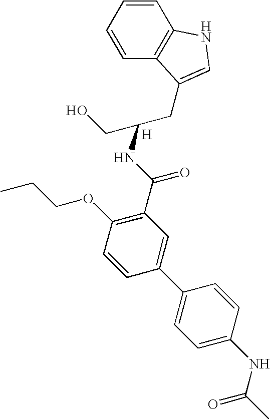 | | | |
| 200 | 4-Propoxy-3′-(2,2,2- trifluoroethoxy)biphenyl-3- carboxylic acid[(R)- 2-hydroxy-1-(1H-indol-3- ylmethyl)ethyl]amide; N-[(R)-1-Hydroxymethyl-2- (1H-indol-3-yl)ethyl]-5- iodo-2-propoxy- benzamide and 3-(2,2,2-Trifluoro- ethoxy)phenyl- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 527.6 Retention time: 10.15 min. | 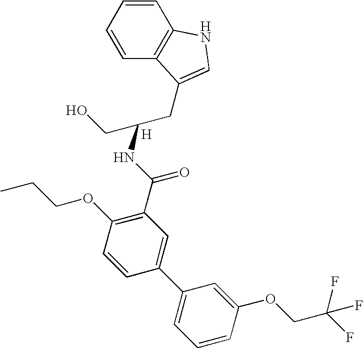 | | | |
| 201 | 3′-Ethoxy-5′-fluoro-4- propoxybiphenyl-3-carboxylic acid [(R)-2-hydroxy-1-(1H- indol-3-ylmethyl)ethyl]- amide; N-[(R)-1- Hydroxymethyl-2-(1H- indol-3-yl)ethyl]-5-iodo-2- propoxybenzamide and 3-Ethoxy-5-fluorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 491.6 Retention time: 10.42 min. | 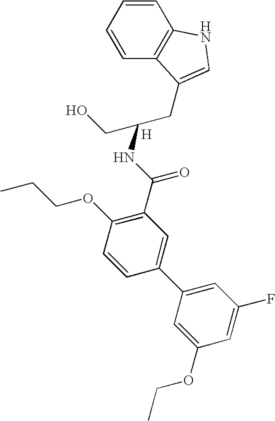 | | | |
| 202 | 5′-Ethoxy-2′-fluoro-4- propoxybiphenyl-3-carboxylic acid [(R)-2-hydroxy-1-(1H- indol-3-ylmethyl)ethyl]amide; N-[(R)-1-Hydroxymethyl-2-(1H- indol-3-yl)ethyl]-5-iodo-2- propoxybenzamide and 5-Ethoxy-2-fluoro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 491.6 Retention time: 10.23 min. | 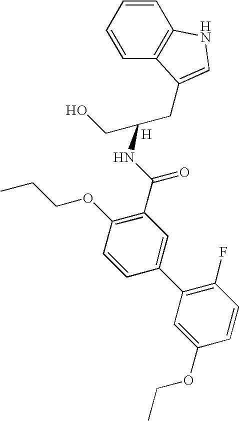 | | | |
| 203 | 3′-Ethoxy-4-propoxybiphenyl-3- carboxylic acid [(R)-1- hydroxymethyl-2-(1H- indol-3-yl)ethyl]amide; N-[(R)-1-Hydroxy- methyl-2-(1H- indol-3-yl)ethyl]-5-iodo-2- propoxybenzamide and 3-Ethoxyphenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 473.6 Retention time: 10.13 min. | 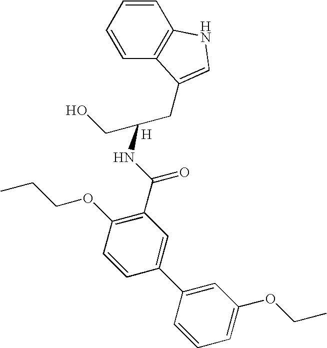 | | | |
| 204 | 4-Propoxybiphenyl-3-carboxylic acid [(R)-1-hydroxymethyl- 20(1-methyl-1H-indol-3- yl)ethyl]amide; N-[(R)-1-Hydroxymethyl-2-(1- methyl-1H-indol-3-yl)ethyl]-5- iodo-2-propoxybenzamide and Phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 443.6 Retention time: 10.76 min. | 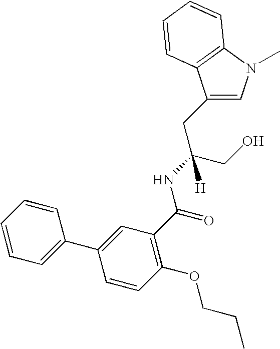 | | | |
| 205 | 5-Benzofuran-2-yl-N-[(R)-1- hydroxymethyl-2-(1-methyl- 1H-indol-3-yl)ethyl]-2- propoxybenzamide; N-[(R)-1-Hydroxymethyl-2-(1- methyl-1H-indol-3-yl)ethyl]-5- iodo-2-propoxybenzamide and Benzo[b]furan- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 483.6 Retention time: 11.22 min. | 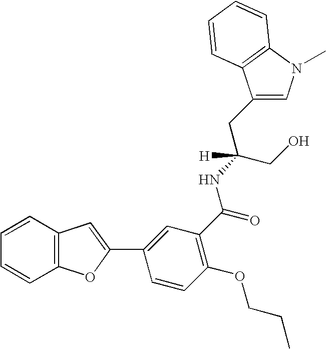 | | | |
| 206 | 5-Benzo[b]thiophen-2-yl-N-[(R)- 2-hydroxy-1-(1-methyl-1H-indol- 3-ylmethyl)ethyl]-2- propoxybenzamide; N-[(R)-1-Hydroxymethyl-2- (1-methyl-1H-indol-3-yl)- ethyl]-5-iodo-2-propoxy- benzamide and Benzo[b]thio- phene-2-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 499.6 Retention time: 11.52 min. | 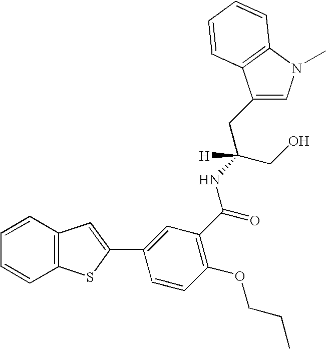 | | | |
| 207 | 2′-Fluoro-4′-methyl-4- propoxybiphenyl-3-carboxylic acid [(R)-2-hydroxy-1-(1-methyl- 1H-indol-3-ylmethyl)- ethyl]amide; N-[(R)-1-Hydroxymethyl-2- (1-methyl-1H-indol-3-yl)- ethyl]-5-iodo-2- propoxybenzamide and 2-Fluoro-4-methyl- phenylboronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 475.6 Retention time: 11.07 min. | 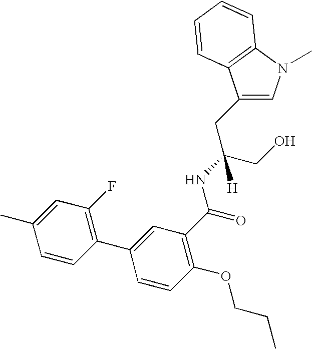 | | | |
| 208 | 4′-Fluoro-4-propoxybiphenyl-3- carboxylic acid [(R)-2-hydroxy- 1-(1-methyl-1H-indol-3- ylmethyl)ethyl]amide; N-[(R)-1-Hydroxymethyl-2- (1-methyl-1H-indol-3-yl)ethyl]-5- iodo-2-propoxybenzamide and 4-Fluorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 461.5 Retention time: 10.61 min. | 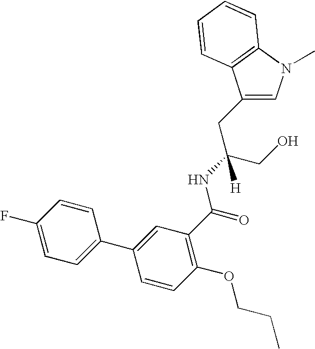 | | | |
| 209 | 2′-Fiuoro-5′-methoxy-4- propoxybiphenyl-3-carboxylic acid [(R)-2-hydroxy-1-(1-methyl- 1H-indol-3-ylmethyl)ethyl]amide; N-[(R)-1-Hydroxymethyl-2-(1- methyl-1H-indol-3-yl)ethyl]-5- iodo-2-propoxybenzamide and 2-Fluoro-5- methoxyphenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 491.6 Retention time: 10.42 min. | 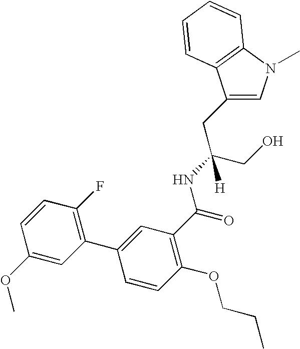 | | | |
| 210 | 3′-Fluoro-4-propoxybiphenyl-3- carboxylic acid [(R)-2-hydroxy- 1-(1-methyl-1H-indol-3- ylmethyl)ethyl]amide; N-[(R)-1Hydroxymethyl- 2-(1-methyl-1H-indol-3- yl)ethyl]-5-methyl-1H- indol-3-yl)ethyl]-5- iodo-2-propoxybenzamide and 3-Fluoro- phenylboronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 461.5 Retention time: 10.63 min. | 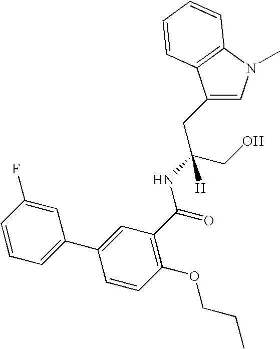 | | | |
| 211 | N-[(R)-1-Hydroxymethyl-2-(1- methyl-1H-indol-3-yl)ethyl]-2- propoxy-5-pyridine-3-yl- benzamide; N-[(R)-1-Hydroxymethyl-2-(1- methyl-1H-indol-3-yl)ethyl]-5- iodo-2-propoxybenzamide and Pyridine- 5-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 444.5 Retention time: 6.73 min. | 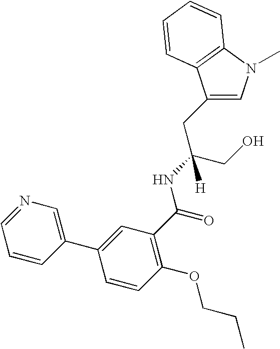 | | | |
| 212 | 5-Benzo[b]thiophene-3-yl-N- [(R)-2-hydroxy-1-(1-methyl-1H- indol-3-ylmethyl)ethyl]-2- propoxybenzamide; N-[(R)-1-Hydroxymethyl-2- (1-methyl-1H-indol-3-yl)- ethyl]-5-iodo-2- propoxybenzamide and Benzo[b]thio- phene-3-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 499.6 Retention time: 11.37 min. | 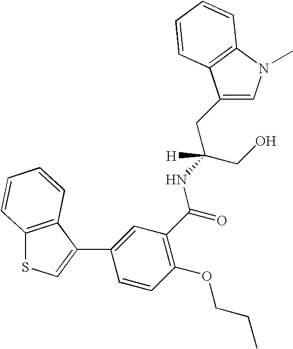 | | | |
| 213 | 3′-Cyano-4′-fluoro-4- propoxybiphenyl-3-carboxylic acid [(R)-2-hydroxy-1- (1-methyl-1H-indol-3-ylmethyl)- ethyl]amide; N-[(R)-1-Hydroxymethyl-2- (1-methyl-1H-indol-3-yl)- ethyl]-5-iodo-2- propoxybenzamide and 4-Fluoro-3-cyano- phenylboronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 486.6 Retention time: 10.16 min. | 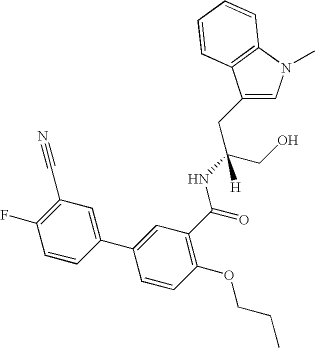 | | | |
| 214 | N-[(R)-1-Hydroxymethyl-2-(1- methyl-1H-indol-3-yl)ethyl]- 5-(6-methoxypyridine- 3-yl)-2-propoxybenzamide; N-[(R)-1-Hydroxymethyl- 2-(1-methyl-1H-indol-3- yl)ethyl]-5-iodo-2- propoxybenzamide and 2-Methoxy- pyridine-5-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 474.6 Retention time: 9.62 min. | 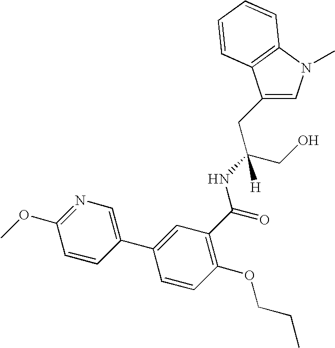 | | | |
| 215 | 3′-Acetylamino-4- propoxybiphenyl-3-carboxylic acid [(R)-1-hydroxy- methyl-2-(1-methyl-1H- indol-3-yl)ethyl]amide; N-[(R)-1-Hydroxymethyl-2- (1-methyl-1H-indol-3- yl)ethyl]-5-iodo-2- propoxybenzamide and 2-Acetamidophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 500.6 Retention time: 9.06 min. | 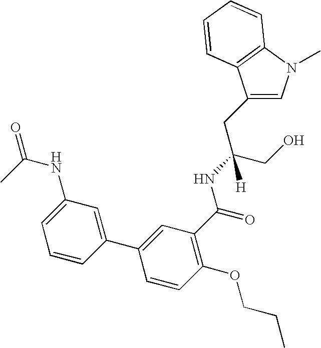 | | | |
| 216 | 3′,4′-Difiuoro-4- propoxybiphenyl- 3-carboxylic acid [(R)-2- hydroxy-1-(1-methyl-1H- indol-3-ylmethyl)- ethyl]amide; N-[(R)-1-Hydroxy- methyl-2-(1- methyl-1H-indol-3- yl)ethyl]-5- iodo-2-propoxy- benzamide and 3,4-Difluorophenyl- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 479.5 Retention time: 10.65 min. | 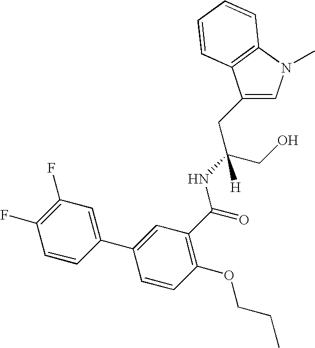 | | | |
| 217 | 3′,5′-Difluoro-4- propoxybiphenyl- 3-carboxylic acid [(R)-2- hydroxy-1-(1-methyl-1H-indol-3- ylmethyl)ethyl]amide; N-[(R)-1-Hydroxy- methyl-2-(1-methyl-1H- indol-3-yl)ethyl]-5- methyl-1H-indol-3- yl)ethyl]-5-iodo-2- propoxybenzamide and 3,5-Difluorophenyl- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 479.5 Retention time: 10.74 min. | 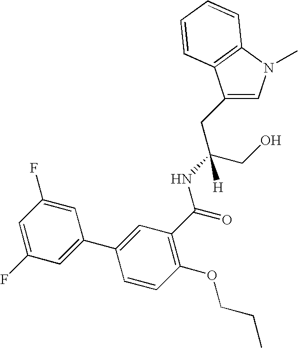 | | | |
| 218 | 5-(2,4-Dimethoxy- pyrimidin-5-yl)- N-[(R)-1-hydroxymethyl-2- (1-methyl-1H-indol-3- yl)ethyl]-2- propoxybenzamide; N-[(R)-1-Hydroxy- methyl-2-(1-methyl- 1H-indol-3-yl)ethyl]-5- iodo-2-propoxybenzamide and 2,4-Dimethoxy- pyrimidine-5- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 505.6 Retention time: 7.76 min. | 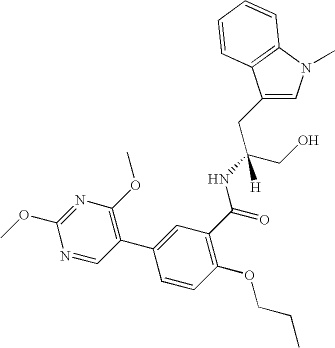 | | | |
| 219 | 2′,5′-Difluoro-4- propoxybiphenyl- 3-carboxylic acid [(R)-2- hydroxy-1-(1-methyl- 1H-indol-3- ylmethyl)ethyl]amide; N-[(R)-1-Hydroxy- methyl-2-(1- methyl-1H-indol-3-yl)ethyl]-5- iodo-2-propoxybenzamide and 2,5-Difluoro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 479.5 Retention time: 10.51 min. | 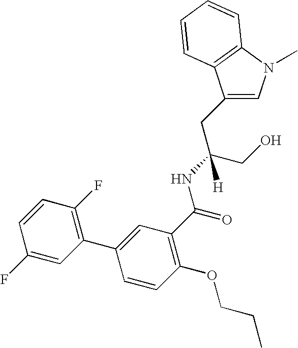 | | | |
| 220 | 5-[(E)-2-(4-Fluoro- phenyl)vinyl]- N-[(R)-2-hydroxy-1- (1-methyl-1H-indol-3- ylmethyl)ethyl]-2- propoxybenzamide; N-[(R)-1-Hydroxy- methyl-2-(1-methyl-1H- indol-3-yl)ethyl]-5- iodo-2-propoxy- benzamide and (E)-2-(4-Fluoro- phenyl)-vinyl- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 487.6 Retention time: 11.07 min. | 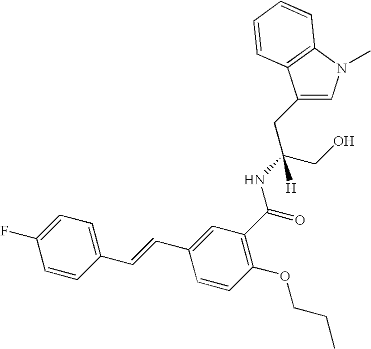 | | | |
| 221 | 5-(5-Cyano-thiophen-2-yl)-N- [(R)-2-hydroxy-1-(1-methyl-1H- indol-3-ylmethyl)ethyl]-2- propoxybenzamide; N-[(R)-1-Hydroxy- methyl-2-(1- methyl-1H-indol-3- 3-yl)ethyl]-5-iodo-2- propoxybenzamide and 5-Cyanothiophene- 2-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 474.6 Retention time: 10.33 min. | 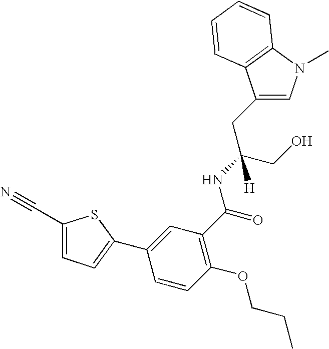 | | | |
| 222 | N-[(R)-1-Hydroxymethyl-2-(1- methyl-1H-indol-3-yl)ethyl]-5-(2- methoxypyrimidine-5-yl)-2- propoxybenzamide; N-[(R)-1-Hydroxymethyl-2-(1- methyl-1H-indol-3-yl)ethyl]-5- iodo-2-propoxybenzamide and 2-Methoxy- pyrimidine-5- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 474.6 Retention time: 8.83 min. | 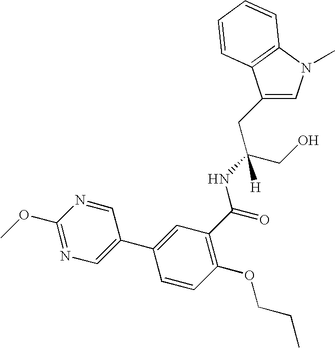 | | | |
| 223 | N-[(R)-1-Hydroxymethyl-2-(1- methyl-1H-indol-3-yl)ethyl]-2- propoxy-5-quinoline-3-yl- benzamide; N-[(R)-1-Hydroxy- methyl-2-(1-methyl-1H- indol-3-yl)ethyl]-5- iodo-2-propoxy- benzamide and Quinoline-3-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 494.6 Retention time: 7.64 min. | 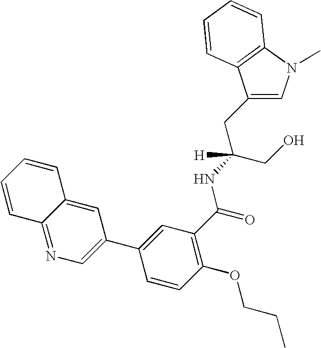 | | | |
| 224 | 5′-Fluoro-3′-methoxy-4- propoxybiphenyl-3-carboxylic acid [(R)-2-hydroxy-1- (1-methyl-1H-indol-3-ylmethyl)- ethyl]amide; N-[(R)-1-Hydroxy- methyl-2-(1-methyl- 1H-indol-3-yl)- ethyl]-5-methyl-1H-indol- 3-yl)ethyl]-5-iodo-2- propoxybenzamide and 3-Fluoro-5- methoxyphenylboronic acid | 135 | | |
| # Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 491.6 Retention time: 10.6 min. | 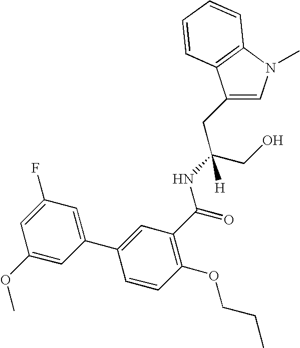 | | | |
| 225 | 4-Propoxy-3′-(2,2,2- trifluoroethoxy)biphenyl-3- carboxylic acid [(R)-2- hydroxy-1-(1-methyl- 1H-indol-3- ylmethyl)ethyl]amide; N-[(R)-1-Hydroxy- methyl-2-(1-methyl-1H- indol-3-yl)ethyl]-5- iodo-2-propoxy- benzamide and 3-(2,2,2-Trifluoro- ethoxy)phenyl- boronic acid | 135 | | |
| # Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 5414.6 Retention time: 10.89 min. | 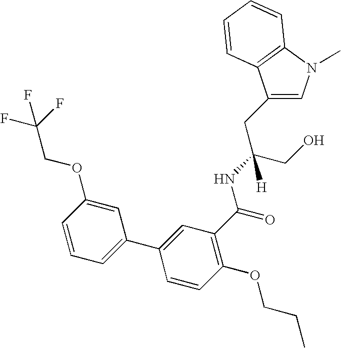 | | | |
| 226 | 5′-Ethoxy-2′-fluoro-4- propoxybiphenyl-3-carboxylic acid [(R)-2-hydroxy-1- (1-methyl-1H-indol-3- ylmethyl)ethyl]amide; N-[(R)-1-Hydroxy- methyl-2-(1-methyl-1H- indol-3-yl)ethyl]-5- iodo-2-propoxybenzamide and 2-Fluoro-5- ethoxyphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 505.6 Retention time: 10.9 min. | 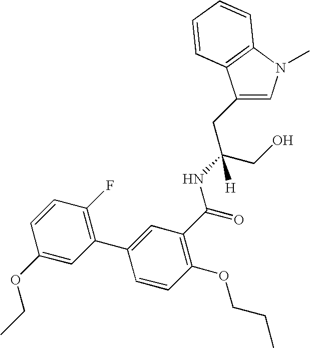 | | | |
| 227 | 4′-Methoxy-4- propoxybiphenyl- 3-carboxylic acid [2- 5-(fluoro-1H-indol-3-yl)-1- hydroxymethylethyl]amide; N-[2-(5-fluoro-1H-indol-3-yl)-1- hydroxymethylethyl]- 5-iodo-2-propoxy- benzamide and 4-Methoxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 477.5 Retention time: 9.78 min. | 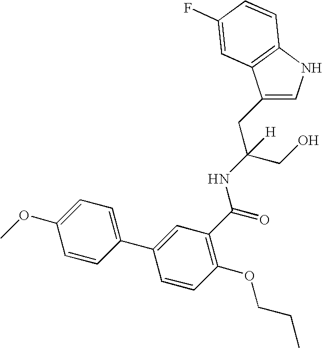 | | | |
| 228 | 5-Benzofuran-2-yl-N-[2-(5- fluoro-1H-indol-3-yl)-1- hydroxymethylethyl]-2- propoxybenzamide; N-[2-(5-Fluoro-1H-indol- 3-yl)-1-hydroxymethyl- ethyl]-5-iodo-2- propoxybenzamide and Benzo[b]furan-2- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 487.5 Retention time: 10.58 min. | 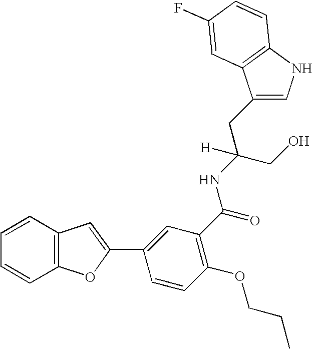 | | | |
| 229 | 3′-Methyl-4-propoxy- biphenyl-3- carboxylic acid [2- (5-fluoro-1H-indol-3-yl)-1- hydroxymethylethyl]amide; N-[2-(5-Fluoro-1H- indol-3-yl)-1-hydroxy- methylethyl]-5-iodo-2- propoxybenzamide and 3-Methylphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 461.5 Retention time: 10.36 min. | 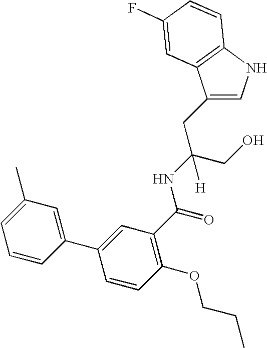 | | | |
| 230 | 5-Benzo[b]thio- phene-2-yl-N-[2- (5-fluoro-1H-indol-3-yl)-1- hydroxymethylethyl]-2- propoxybenzamide; N-[2-(5-Fluoro-1H- indol-3-yl)-1- hydroxymethylethyl]- 5-iodo-2-propoxy- benzamide and Benzo[b]thiophene- 2-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 503.6 Retention time: 10.92 min. | 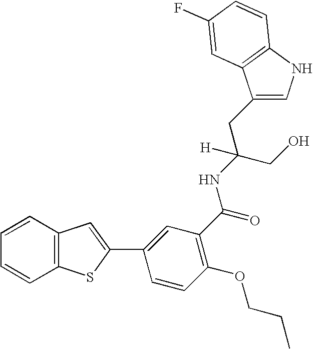 | | | |
| 231 | 2′-Eluoro-5′-methoxy-4- propoxybiphenyl-3-carboxylic acid [1-(5-fluoro-1H-indol-3- ylmethyl)-2-hydroxyethyl]amide; N-[2-(5-Fluoro-1H-indol-3-yl)-1- hydroxymethylethyl]-5-iodo-2- propoxybenzamide and 2-Fluoro-5-methoxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 495.5 Retention time: 9.86 min. | 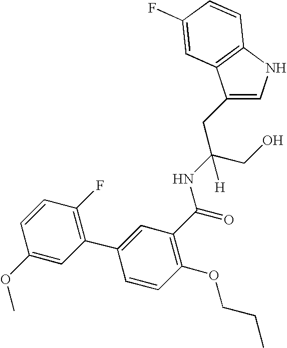 | | | |
| 232 | 4-Propoxy-3′,5′-bis- trifluoromethylbiphenyl-3- carboxylic acid [1-(5-fluoro- 1H-indol-3-ylmethyl)-2- hydroxyethyl]amide; N-[2-(5-Fluoro-1H-indol- 3-yl)-1-hydroxy- methylethyl]-5-iodo-2- propoxybenzamide and 3,5-Bistrifluoromethyl- phenylboronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 583.5 Retention time: 10.97 min. | 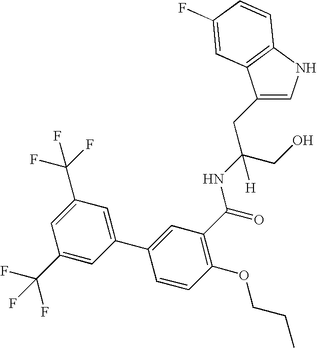 | | | |
| 233 | N-[2-(5-Fluoro-1H- indol-3-yl)-1- hydroxymethylethyl]-2- propoxy-5-pyridin-3-yl- benzamide; N-[2-(5-Fluoro-1H-indol- 3-yl)-1-hydroxy- methylethyl]-5-iodo-2- propoxybenzamide and Pyridine-3-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 448.5 Retention time: 6.26 min. | 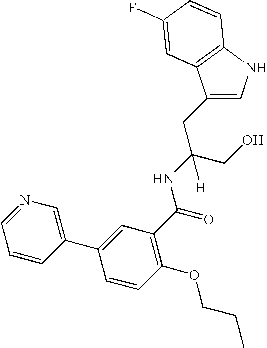 | | | |
| 234 | 5-Benzo[b]thio- phene-3-yl-N-[2- (5-fluoro-1H-indol-3-yl)-1- hydroxymethylethyl]-2- propoxybenzamide; N-[2-(5-Fluoro-1H- indol-3-yl)-1- hydroxymethylethyl]-5- iodo-2-propoxybenzamide and Benzo[b]thio- phene-3-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 503.6 Retention time: 10.63 min. | 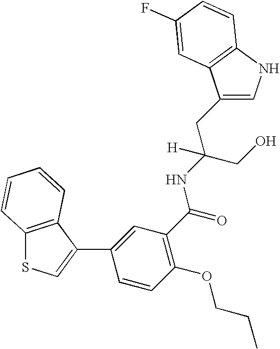 | | | |
| 235 | 3′-Cyano-4′-fluoro-4- propoxybiphenyl-3-carboxylic acid [1-(5-fluoro-1H-indol-3- ylmethyl)-2-hydroxy- ethyl]amide; N-[2-(5-Fluoro-1H- indol-3-yl)-1-hydroxy- methylethyl]-5-iodo-2- propoxybenzamide and 3-Cyano-4-fluorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 490.5 Retention time: 9.52 min. | 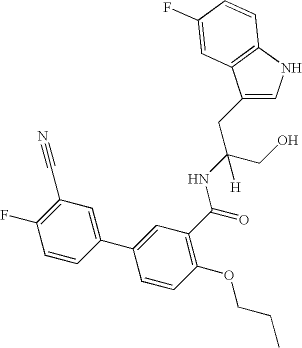 | | | |
| 236 | N-[1-(5-Fluoro-1H-indol-3- ylmethyl)-2-hydroxymethyl]- 5-(6-fluoro-5- methylpyridine-3-yl)-2- propoxybenzamide; N-[2-(5-Fluoro-1H-indol- 3-yl)-1-hydroxy- methylethyl]-5-iodo-2- propoxybenzamide and 2-Fluoro-3-methyl- pyridine-5- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 480.5 Retention time: 9.26 min. | 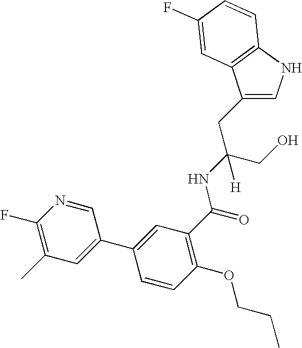 | | | |
| 237 | N-[2-(5-Fluoro-1H- indol-3-yl)-1- hydroxymethylethyl]-5-(6- methoxypyridine-3-yl)-2- propoxybenzamide; N-[2-(5-Fluoro-1H- indol-3-yl)-1- hydroxymethyl- ethyl]-5-iodo-2- propoxybenzamide and 2-Methoxypyridine-5- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 478.5 Retention time: 8.77 min. | 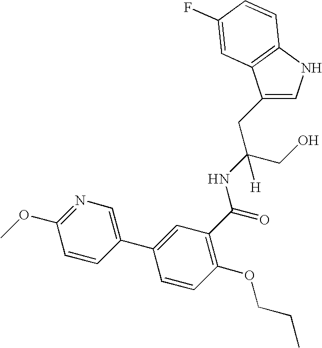 | | | |
| 238 | 3′-Chloro-4′-fluoro-4- propoxybiphenyl-3-carboxylic acid [1-(5-fluoro-1H- indol-3-ylmethyl)- 2-hydroxyethyl]amide; N-[2-(5-Fluoro- 1H-indol-3-yl)-1- hydroxymethylethyl]-5- iodo-2-propoxybenzamide and 3-Chloro-4-fluoro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 500 Retention time: 10.54 min. | 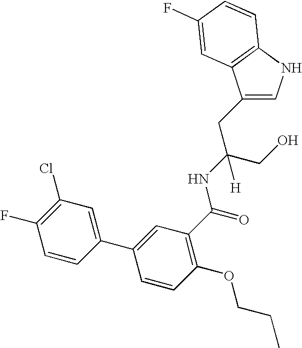 | | | |
| 239 | 3′-Acetylamino-4- propoxybiphenyl-3-carboxylic acid [2-(5-fluoro-1H-indol-3- yl)-1-hydroxymethyl- ethyl]-5-iodo-2- propoxybenzamide and 3-Acetamidophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 504.6 Retention time: 8.37 min. | 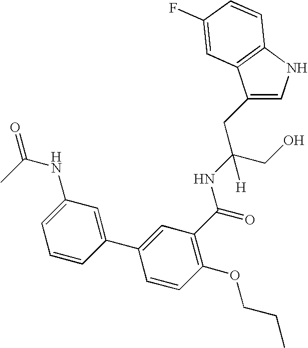 | | | |
| 240 | 3′,4′-Difluoro-4-propoxy- biphenyl-3-carboxylic acid [1-(5-fluoro-1H- indol-3-ylmethyl)-2- hydroxyethyl]amide; N-[2-(5-Fluoro-1H- indol-3-yl)-1-hydroxy- methylethyl]-5-iodo-2- propoxybenzamide and 3,4-Difluorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 483.5 Retention time: 10.08 min. | 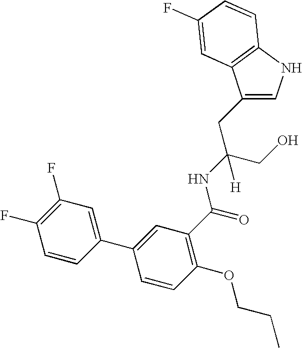 | | | |
| 241 | 3′,5′-Difluoro-4- propoxybiphenyl- 3-carboxylic acid [2-(5-fluoro-1H-indol-3-yl)-1- hydroxymethylethyl]amide; N-[2-(5-fluoro-1H-indol-3-yl)-1- hydroxymethylethyl]- 5-iodo-2-propoxy- benzamide and 3,5-Difluoro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 483.5 Retention time: 10.07 min. | 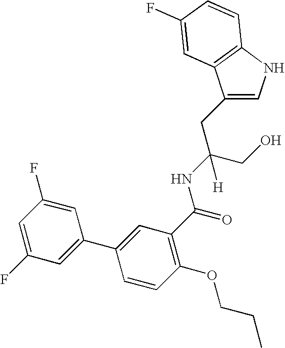 | | | |
| 242 | 2′,5′-Difluoro- 4-propoxybiphenyl- 3-carboxylic acid [1-(5-fluoro-1H-indol-3- ylmethyl)-2-hydroxy- ethyl]amide; N-[2-(5-Fluoro-1H-indol- 3-yl)-1-hydroxy- methylethyl]-5-iodo-2- propoxybenzamide and 2,5-Difluoro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 483.5 Retention time: 9.95 min. | 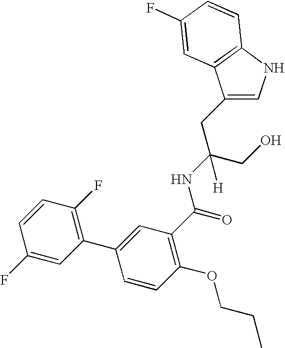 | | | |
| 243 | N-[2-(5-Fluoro-1H- indol-3-yl)-1- hydroxymethyl- ethyl]-5-[(E)-2-(4- fluorophenyl)-vinyl]-2- propoxybenzamide; N-[2-(5-Fluoro-1H-indol- 3-yl)-1-hydroxy- methylethyl]-5-iodo-2- propoxybenzamide and (E)-2-(4-Fluorphenyl)vinyl- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 491.5 Retention time: 10.46 min. | 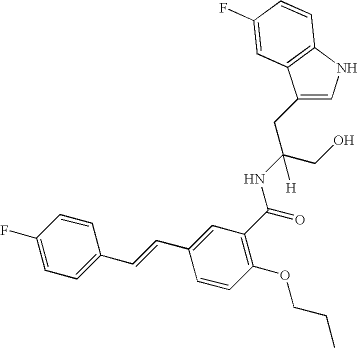 | | | |
| 244 | 5-(5-Cyano-thiophene-2-yl)-N- [2-(5-fluoro-1H-indol-3-yl)-1- hydroxymethylethyl]-2- propoxybenzamide; N-[2-(5-Fluoro-1H-indol- 3-yl)-1- hydroxymethyl- ethyl]-5-iodo-2- propoxybenzamide and 5-Cyanothiophene- 2-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 478.6 Retention time: 9.5 min. | 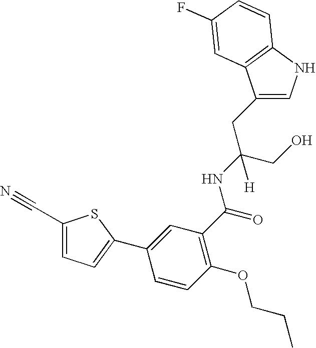 | | | |
| 245 | 2′-Fluoro-3′-methoxy-4- propoxybiphenyl-3- carboxylic acid [1-(5- fluoro-1H-indol-3- ylmethyl)-2-hydroxy- ethyl]amide; N-[2-(5-Fluoro-1H-indol- 3-yl)-1-hydroxy- methylethyl]-5-iodo-2- propoxybenzamide and 2-Fluoro-3-methoxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 495.5 Retention time: 9.57 min. | 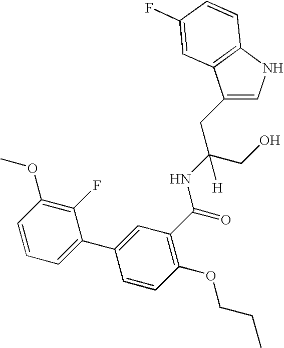 | | | |
| 246 | N-[2-(5-Fluoro-1H- indol-3-yl)-1- hydroxymethylethyl]-5-(2- methoxypyrimidin-5-yl)-2- propoxybenzamide; N-[2-(5-Fluoro-1H-indol-3- yl)-1-hydroxymethyl- ethyl]-5-iodo-2- propoxybenzamide and 2-Methoxypyrimidine- 5-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 479.5 Retention time: 8.27 min. | 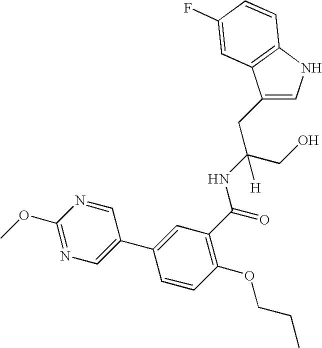 | | | |
| 247 | N-[2-(5-Fluoro-1H- indol-3-yl)-1- hydroxymethylethyl]- 2-propoxy-5- quinoline-3-yl-benzamide; N-[2-(5-Fluoro-1H-indol- 3-yl)-1-hydroxy- methylethyl]-5-iodo-2- propoxybenzamide and Quinoline-3-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 498.6 Retention time: 7.05 min. | 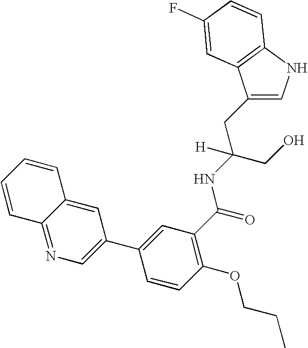 | | | |
| 248 | 4-Propoxy-3′-(2,2,2- trifluoroethoxy)biphenyl-3- carboxylic acid [1- (5-fluoro-1H-indol-3-ylmethyl)-2- hydroxyethyl]amide; N-[2-(5-Fluoro-1H-indol- 3-yl)-1-hydroxy- methylethyl]-5-iodo-2- propoxybenzamide and 3-(2,2,2-Trifluoroethoxy)- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 545.5 Retention time: 10.27 min. | 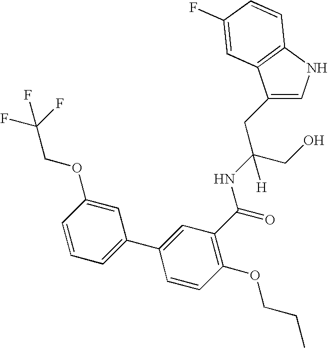 | | | |
| 249 | 5′-Ethoxy-2′-fluoro-4- propoxybiphenyl-3- carboxylic acid [1-(5- fluoro-1H-indol-3- ylmethyl)-2-hydroxy- ethyl]amide; N-[2-(5-Fluoro-1H- indol-3-yl)-1-hydroxy- methylethyl]-5-iodo-2- propoxybenzamide and 2-Fluoro-5-ethoxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 509.6 Retention time: 10.33 min. | 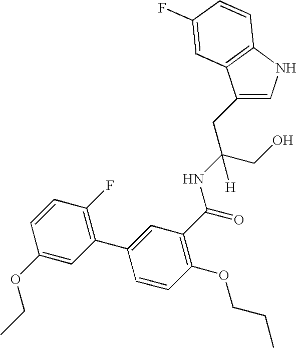 | | | |
| 250 | 3′-Methoxy-4-propoxy- biphenyl-3-carboxylic acid [(R)-1-hydroxymethyl- 2-(1-methyl-1H- indol-3-yl)ethyl]amide; N-[(R)-1-Hydroxymethyl- 2-(1-methyl-1H- indol-3-yl)ethyl]-5- iodo-2-propoxy- benzamide and 3-Methoxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 473.6 Retention time: 10.31 min. | 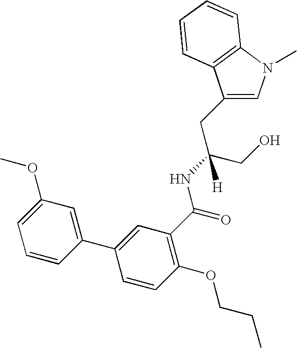 | | | |
| 251 | 3′-Chloro-4-propoxy- biphenyl-3-carboxylic acid [(R)-2-hydroxy-1-(1- methyl-1H-indol-3- ylmethyl)ethyl]amide; N-[(R)-1-Hydroxymethyl- 2-(1-methyl-1H-indol- 3-yl)ethyl]-5- iodo-2-propoxybenzamide and 3-Chloro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 478 Retention time: 11.14 min. | 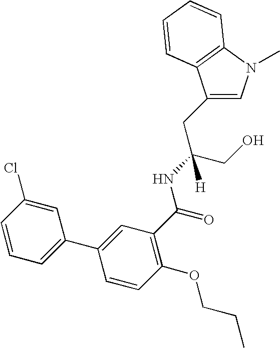 | | | |
| 252 | 4-Propoxy-3′,5′-bis- trifluoromethylbiphenyl-3- carboxylic acid [(R)- 2-hydroxy-1-(1-methyl- 1H-indol-3-ylmethyl)- ethyl]amide; N-[(R)-1-Hydroxymethyl-2-(1- methyl-1H-indol-3-yl)- ethyl]-5-iodo-2- propoxybenzamide and 3,5-Bistrifluoro- methylphenylboronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 579.6 Retention time: 11.68 min. | 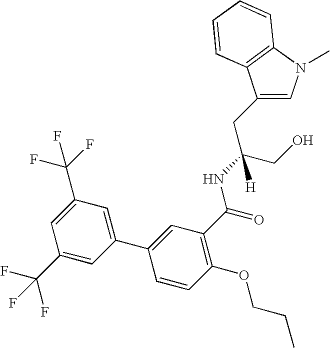 | | | |
| 253 | 3′,4′,5′-Trifluoro-4- propoxybiphenyl-3-carboxylic acid [(R)-2-hydroxy-1- (1-methyl- 1H-indol-3-ylmethyl)- ethyl]amide; N-[(R)-1-Hydroxymethyl- 2-(1-methyl-1H-indol-3- yl)ethyl]-5- iodo-2-propoxybenzamide and 3,4,5-Trifluorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 497.5 Retention time: 11.01 min. | 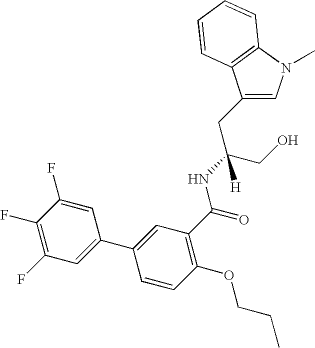 | | | |
| 254 | 4-Propoxy-4′- trifluoromethoxybiphenyl-3- carboxylic acid [(R)- 2-hydroxy-1-(1-methyl-1H- indol-3-ylmethyl)- ethyl]amide; N-[(R)-1-Hydroxymethyl- 2-(1-methyl-1H-indol-3- yl)ethyl]-5-iodo-2- propoxybenzamide and 4-Trifluoromethoxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 527.6 Retention time: 11.2 min. | 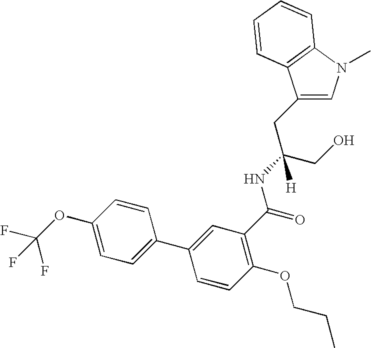 | | | |
| 255 | 4-Propoxy-4′- trifluoromethylbiphenyl-3- carboxylic acid [(R)- 2-hydroxy-1-(1-methyl-1H-indol-3- ylmethyl)ethyl]amide; N-[(R)-1-Hydroxymethyl-2- (1-methyl-1H-indol-3-yl)ethyl]- 5-iodo-2-propoxy- benzamide and 4-Trifluoromethyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 511.6 Retention time: 11.00 min. | 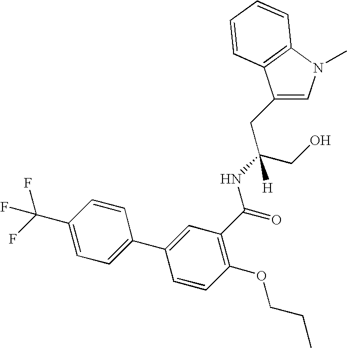 | | | |
| 256 | 5-(6-Fluoro-5-methyl- pyridine-3-yl)-N-[(R)- 2-hydroxy-1-(1-methyl- 1H-indol-3-ylmethyl)ethyl]-2- propoxybenzamide; N-[(R)-1-Hydroxymethyl- 2-(1-methyl-1H-indol-3- yl)ethyl]-5-iodo-2- propoxybenzamide and 2-Fluoro-3-methylpyridine- 5-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 476.6 Retention time: 10.02 min. | 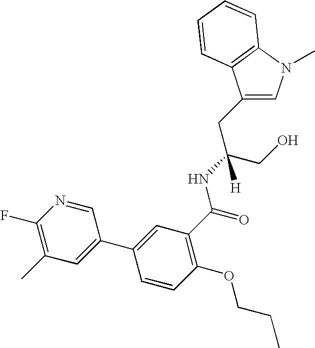 | | | |
| 257 | 5-(3,5-Dimethyl- isoxazol-4-yl)- N-[(R)-1-hydroxy- methyl-2-(1- methyl-1H-indol-3-yl)- ethyl]-2-propoxy- benzamide; N-[(R)-1-Hydroxymethyl-2- (1-methyl-1H-indol-3- yl)ethyl]-5- iodo-2-propoxy- benzamide and 3,5-Dimethyl- isoxazole-4-boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 462.6 Retention time: 9.32 min. | 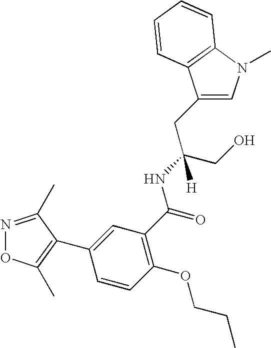 | | | |
| 258 | 3′-Chloro-4′-fluoro-4- propoxybiphenyl-3-carboxylic acid [(R)-2-hydroxy-1- (1-methyl-1H-indol-3- ylmethyl)ethyl]amide; N-[(R)-1-Hydroxymethyl- 2-(1-methyl-1H-indol- 3-yl)ethyl]-5-iodo-2- propoxybenzamide and 3-Chloro-4-fluoro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 496 Retention time: 11.22 min. | 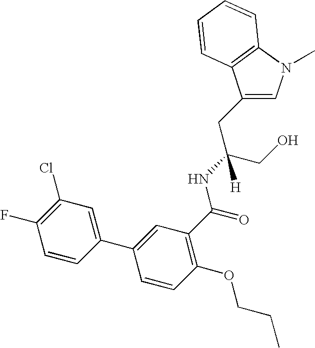 | | | |
| 259 | 3′-Cyano-4-propoxy- biphenyl-3- carboxylic acid [(R)-1- hydroxymethyl-2- (1-methyl-1H-indol-3- yl)ethyl]amide; N-[(R)-1-Hydroxymethyl- 2-(1-methyl-1H-indol-3- yl)ethyl]-5- iodo-2-propoxy- benzamide and 3-Cyanophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 468.6 Retention time: 10.1 min. | 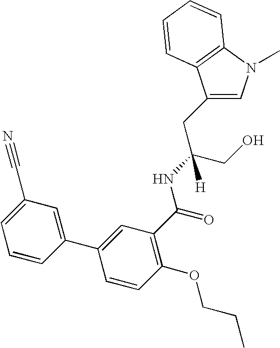 | | | |
| 260 | 2′,3′-Difluoro-4- propoxybiphenyl- 3-carboxylic acid [(R)-2- hydroxy-1-(1-methyl- 1H-indol-3- ylmethyl)ethyl]amide; N-[(R)-1-Hydroxymethyl- 2-(1-methyl-1H- indol-3-yl)- ethyl]-5-iodo-2- propoxybenzamide and 2,3-Difluorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 479.5 Retention time: 10.54 min. | 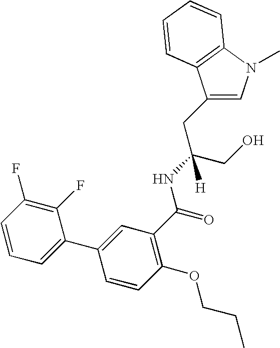 | | | |
| 261 | 3′,5′-Dimethyl-4- propoxybiphenyl-3- carboxylic acid [(R)-1- hydroxymethyl-2- (1-methyl-1H-indol-3- yl)ethyl]amide; N-[(R)-1-Hydroxy- methyl-2-(1- methyl-1H-indol-3-yl)ethyl]-5- iodo-2-propoxy- benzamide and 3,5-Dimethylphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 471.6 Retention time: 10.18 min. | 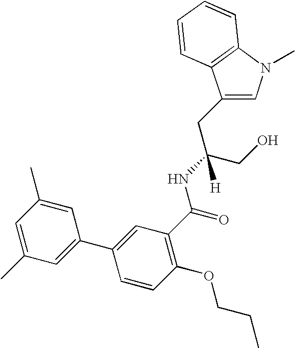 | | | |
| 262 | 3′-Ethoxy-5′-fluoro-4- propoxybiphenyl-3- carboxylic acid [(R)- 2-hydroxy-1-(1- methyl-1H-indol-3-ylmethyl)- ethyl]amide; N-[(R)-1-Hydroxymethyl- 2-(1-methyl-1H-indol-3- yl)ethyl]-5- iodo-2-propoxybenzamide and 5-Ethoxy-3-fluoro- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 505.6 Retention time: 10.98 min. | 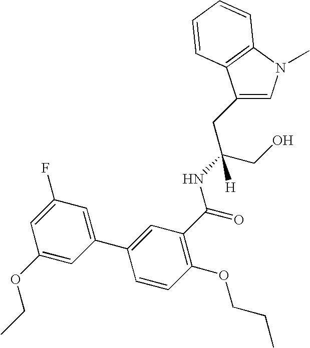 | | | |
| 263 | 5′-Fluoro-3′-hydroxy-4- propoxybiphenyl-3-carboxylic acid [(R)-2-hydroxy- 10(1-methyl-1H-indol-3-ylmethyl)- ethyl]amide; N-[(R)-1-Hydroxymethyl-2- (1-methyl-1H-indol-3- yl)ethyl]-5-iodo-2- propoxybenzamide and 3-Fluoro-5- hydroxyphenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 477.5 Retention time: 9.5 min. | 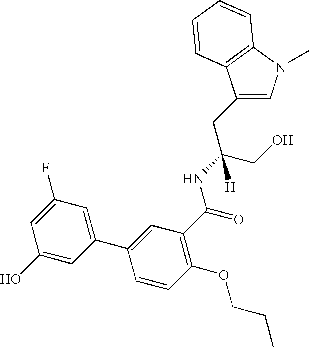 | | | |
| 264 | 4,3′-Dipropoxybiphenyl-3- carboxylic acid [(R)-1- hydroxymethyl-2-(1- methyl-1H-indol-3- yl)ethyl]amide; N-[(R)-1-Hydroxymethyl-2- (1-methyl-1H-indol-3- yl)ethyl]-5-iodo-2- propoxybenzamide and 3-Propoxyphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 501.6 Retention time: 11.21 min. | 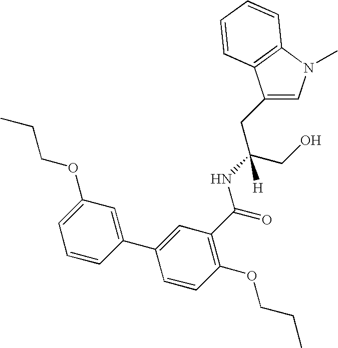 | | | |
| 265 | 3′-Chloro-4-propoxybiphenyl-3- carboxylic acid [2-(5- fluoro-1H-indol-3-yl)-1- hydroxymethylethyl]amide; N-[2-(5-Fluoro-1H-indol- 3-yl)-1-hydroxymethyl- ethyl]-5-iodo-2- propoxybenzamide and 3-Chlorophenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 482 Retention time: 10.42 min. | 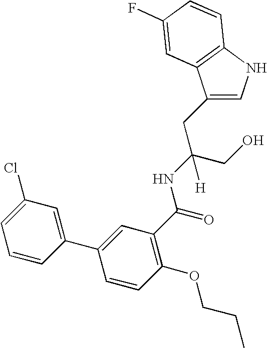 | | | |
| 266 | 3′-Fluoro-4′-methyl-4- propoxybiphenyl-3-carboxylic acid [1-(5-fluoro-1H-indol-3- ylmethyl)-2-hydroxyethyl]amide; N-[2-(5-Fluoro-1H-indol-3-yl)-1- hydroxymethylethyl]-5-iodo-2- propoxybenzamide and 3-Fluoro-4-methylphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 479.5 Retention time: 10.08 min. | 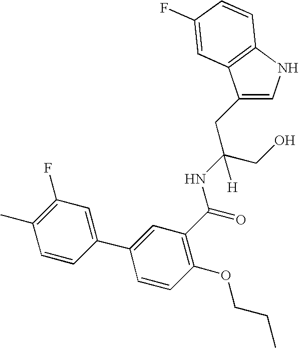 | | | |
| 267 | 2′-Fluoro-4′-methyl-4- propoxybiphenyl-3-carboxylic acid [1-(5-fluoro-1H-indol-3- ylmethyl)-2-hydroxyethyl]amide; N-[2-(5-Fluoro-1H- indol-3-yl)-1- hydroxymethyl- ethyl]-5-iodo-2- propoxybenzamide and 2-Fluoro-4-methylphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 479.5 Retention time: 10.27 min. | 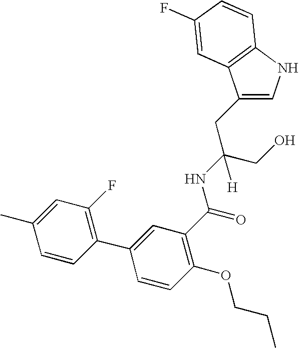 | | | |
| 268 | 4-Propoxy-3′- trifluoromethylbiphenyl-3- carboxylic acid [1- (5-fluoro-1H- indol-3-ylmethyl)-2- hydroxyethyl]amide; N-[2-(5-Fluoro-1H-indol-3- yl)-1-hydroxymethyl- ethyl]-5-iodo-2- propoxybenzamide and 3-Trifluoromethyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 515,5 Retention time: 10,41 min. | 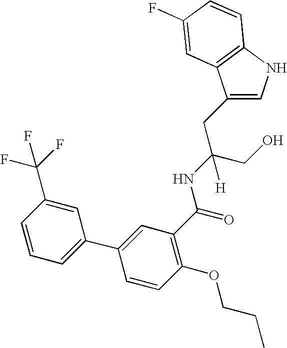 | | | |
| 269 | 3′-Isopropyl-4-propoxybiphenyl- 3-carboxylic acid [2- (5-fluoro-1H-indol-3-yl)-1- hydroxymethylethyl]amide; N-[2-(5-Fluoro-1H-indol- 3-yl)-1-hydroxy- methylethyl]-5-iodo-2- propoxybenzamide and 3-Isopropylphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 489.6 Retention time: 10.96 min. | 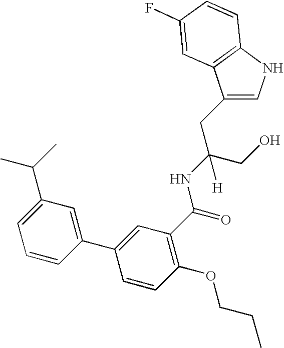 | | | |
| 270 | 3′-Methylsulphanyl-4- propoxybiphenyl-3-carboxylic acid [2-(5-fluoro-1H-indol 3-yl)-1-hydroxymethyl- ethyl]amide; N-[2-(5-Fluoro-1H-indol- 3-yl)-1-hydroxy- methylethyl]-5-iodo-2- propoxybenzamide and 3-Methylsulphanyl- phenylboronic acid acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 493.6 Retention time: 10.31 min. | 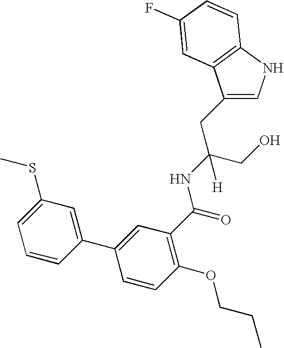 | | | |
| 271 | 4-Propoxy-4′- trifluoromethoxy- biphenyl-3- carboxylic acid [1-(5- fluoro-1H- indol-3-ylmethyl)-2- hydroxyethyl]amide; N-[2-(5-Fluoro-1H-indol- 3-yl)-1-hydroxy- methylethyl]-5-iodo-2- propoxybenzamide and 4-Trifluoromethoxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 531.5 Retention time: 10.69 min. | 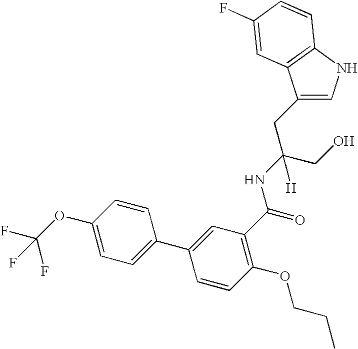 | | | |
| 272 | N-[2-(5-Fluoro-1H- indol-3-yl)-1- hydroxymethyl- ethyl]-2-propoxy- 5-quinoline-6-yl- benzamide; N-[2-(5-Fluoro-1H-indol- 3-yl)-1-hydroxymethyl- ethyl]-5-iodo-2- propoxybenzamide and Quinoline-6-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 498.6 Retention time: 6.9 min. | 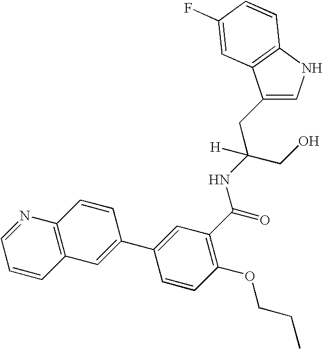 | | | |
| 273 | 3′-Chloro-4′-methyl-4- propoxybiphenyl-3-carboxylic acid [2-(5-fluoro-1H-indol- 3-yl)-1-hydroxy- methylethyl]amide; N-[2-(5-Fluoro-1H-indol-3- yl)-1-hydroxymethyl- ethyl]-5-iodo-2- propoxybenzamide and 3-Chloro-4-methyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 496 Retention time: 11.03 min. | 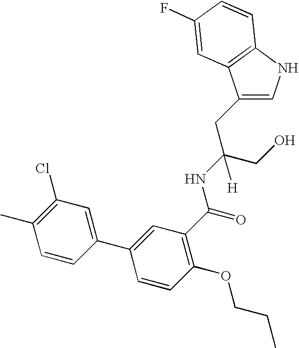 | | | |
| 274 | 5-(3,5-Dimethyl-isoxazol-4-yl)- N-[2-(5-fluoro-1H-indol-3-yl)-1- hydroxymethylethyl]-2- propoxybenzamide; N-[2-(5-Fluoro-1H-indol-3-yl)- 1-hydroxymethylethyl]- 5-iodo-2-propoxy- benzamide and 3,5-Dimethylisoxazole- 4-boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 466.5 Retention time: 8.77 min. | 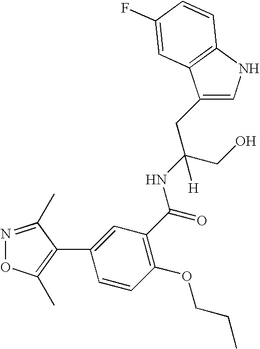 | | | |
| 275 | 2′,3′-Difluoro-4- propoxybiphenyl- 3-carboxylic acid [1- (5-fluoro-1H-indol-3- ylmethyl)-2- hydroxyethyl]amide; N-[2-(5-Fluoro-1H-indol-3- 3-yl)-1-hydroxymethyl- ethyl]-5-iodo-2-hydroxy- methylethyl]-5-iodo-2- propoxybenzamide and 2,3-Difluorophenyl- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 483.5 Retention time: 10.05 min. | 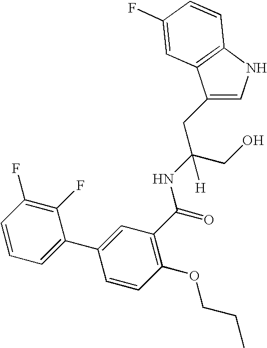 | | | |
| 276 | 3′,5′-Dimethyl-4- propoxybiphenyl-3-carboxylic acid [2-(5-fluoro-1H-indol- 3-yl)-1-hydroxymethyl- ethyl]amide; N-[2-(5-Fluoro-1H-indol- 3-yl)-1-hydroxy- methylethyl]-5-iodo-2- propoxybenzamide and 3,5-Dimethylphenyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 475.6 Retention time: 10.71 min. | 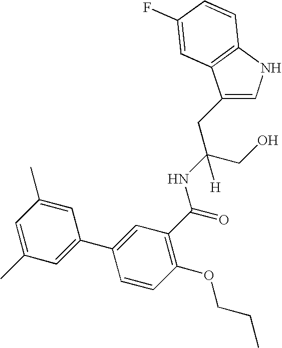 | | | |
| 277 | 5′-Ethoxy-3′-fluoro-4- propoxybiphenyl-3-carboxylic acid [2-(5-fluoro-1H-indol- 3-yl)-1-hydroxymethyl- ethyl]amide; N-[2-(5-Fluoro-1H-indol- 3-yl)-1-hydroxymethyl- ethyl]-5-iodo-2- propoxybenzamide and 5-Ethoxy-3-fluorphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 509.6 Retention time: 10.41 min. | 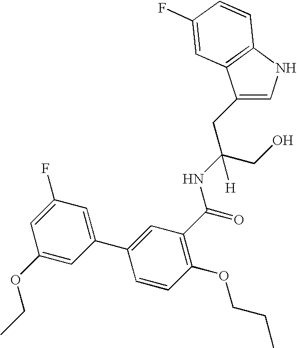 | | | |
| 278 | 3′-Fluoro-5′-hydroxy-4- propoxybiphenyl-3-carboxylic acid [2-(5-fluoro-1H- indol-3-yl)-1-hydroxy- methylethyl]amide; N-[2-(5-Fluoro-1H-indol-3- yl)-1-hydroxymethyl- ethyl]-5-iodo-2- propoxybenzamide and 3-Fluoro-5-hydroxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 481.5 Retention time: 9.03 min. | 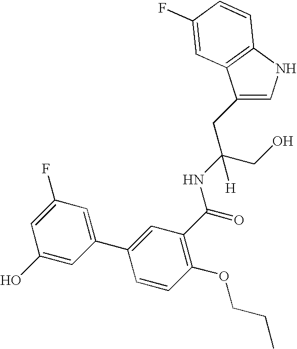 | | | |
| 279 | 4,3′-Dipropoxybiphenyl-3- carboxylic acid [2-(5-fluoro-1H- indol-3-yl)-1-hydroxy- methylethyl]amide; N-[2-(5-Fluoro-1H-indol- 3-yl)-1-hydroxy- methylethyl]-5-iodo-2- propoxybenzamide and 3-n-Propoxy- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 505.6 Retention time: 10.67 min. | 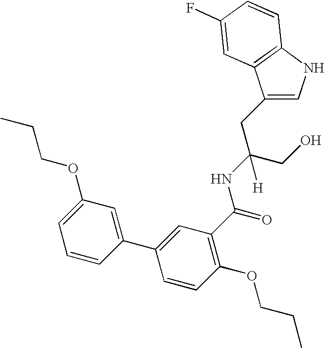 | | | |
| 280 | 3′-Ethoxy-4-propoxy- biphenyl-3- carboxylic acid [2- (5-fluoro-1H- indol-3-yl)-1-hydroxy- methylethyl]amide; N-[2-(5-Fluoro-1H-indol- 3-yl)-1-hydroxymethyl- ethyl]-5-iodo-2- propoxybenzamide and 3-Ethoxyphenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 491.6 Retention time: 10.29 min. | 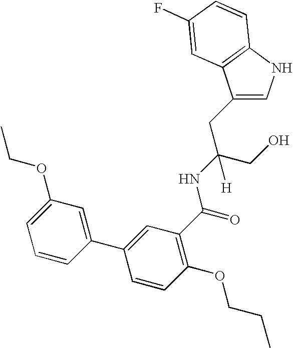 | | | |
| 281 | 4′-Hydroxymethyl-4- propoxybiphenyl-3-carboxylic acid [(R)-1-hydroxy- methyl-2-(1H-indol- 3-yl)ethyl]amide; N-[(R)-1-Hydroxy- methyl-2-(1H- indol-3-yl)ethyl]-5-iodo-2- propoxybenzamide and 4-(Hydroxymethyl)- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 459.5 Retention time: 8.16 min. | 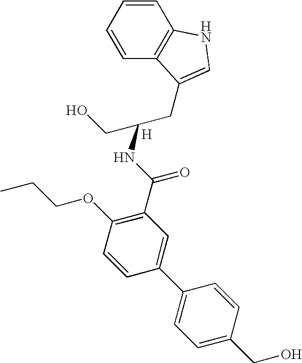 | | | |
| 282 | 3′-Hydroxymethyl-4- propoxybiphenyl-3-carboxylic acid [(R)-1-hydroxy- methyl-2- (1H-indol-3-yl)ethyl]amide; N-[(R)-1-Hydroxymethyl-2-(1H- indol-3-yl)ethyl]-5-iodo-2- propoxybenzamide and 4-(hydroxymethyl)- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 459.5 Retention time: 8.28 min. | 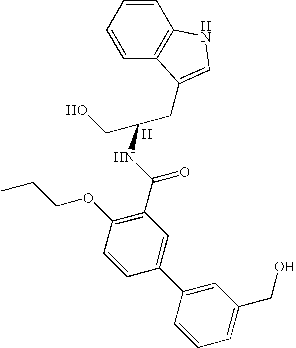 | | | |
| 283 | 4-Propoxybiphenyl-3,4′- dicarboxylic acid 3-{[(R)-1- hydroxymethyl-2-(1H- indol-3-yl)ethyl]amide}4′- methylamide; N-[(R)-2-Hydroxy-1-(1H-indol- 3-ylmethyl)ethyl]-5-iodo-2- propoxybenzamide and 4-(N-Methylamino- carbonyl)-phenyl- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 486.5 Retention time: 7.84 min. | 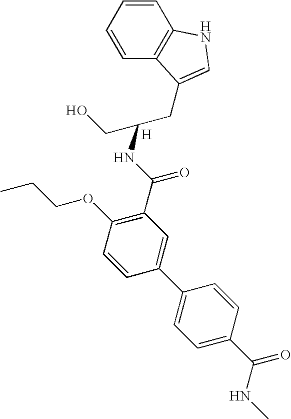 | | | |
| 284 | N-[(R)-2-Hydroxy-1- (1H-indol-3- ylmethyl)ethyl]-5-(5-hydroxy- methylthio- phene-2-yl)-2-propoxy- benzamide; N-[(R)-2-Hydroxy-1-(1H- indol-3-ylmethyl)ethyl]-5-iod-2- propoxybenzamide and 5-(Hydroxymethyl)- thiophene-2- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 465.5 Retention time: 8.21 min. | 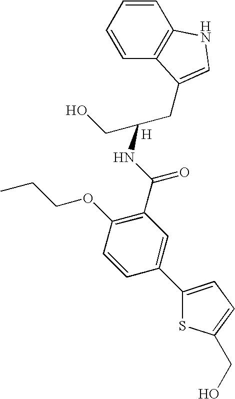 | | | |
| 285 | 5′-Fluoro-4-propoxy- biphenyl- 3,3′-dicarboxylic acid 3-{[(R)-2-hydroxy-1- (1H-indol-3- ylmethyl)ethyl]amide}3′-methyl-amide; N-[(R)-2-Hydroxy-1-(1H- indol-3-ylmethyl)ethyl]-5- iodo-2-propoxy- benzamide and 3-Fluoro-5-(methyl- carbanoyl)- phenylboronic acid | 135 | | |
| # Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 504.5 Retention time: 8.46 min. | 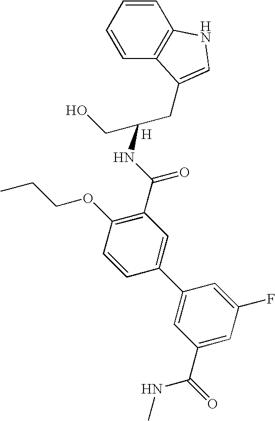 | | | |
| 286 | 3′-Chloro-4-propoxy- biphenyl-3,4′- dicarboxylic acid 3-{[(R)-2- hydroxy-1-(1H-indol-3- ylmethyl)ethyl]amide}4′- methylamide; N-[(R)-2-Hydroxy-1-(1H-indol-3- ylmethyl)ethyl]-5-iodo-2- propoxybenzamide and 3-Chloro-4-(N- methylcarbamoyl)phenyl- boronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 521 Retention time: 8.11 min. | 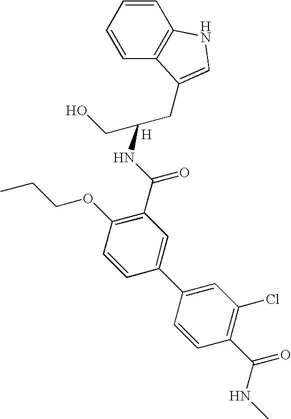 | | | |
| 287 | 3′-Hydroxymethyl-4- propoxybiphenyl-3- carboxylic acid [(R)-1- hydroxymethyl-2-(1- methyl-1H-indol-3- yl)ethyl]amide; N-[(R)-1-Hydroxy- methyl-2-(1-methyl- 1H-indol-3-yl)ethyl]- 5-iodo-2- propoxybenzamide and 3-Hydroxymethyl- phenylboronic acid | 135 | Column Purospher Star RP | |
| # C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 473.6 Retention time: 8.92 min. | 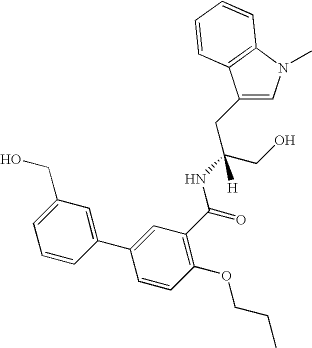 | | | |
| 288 | 3′-Hydroxymethyl-4- propoxybiphenyl-3- carboxylic acid [2-(5- fluoro-1H-indol-3-yl)- 1-hydroxymethylethyl]amide; N-[2-(5-Fluoro-1H- indol-3-yl)-1-hydroxy- methylethyl]-5-iodo-2- propoxybenzamide and 3-Hydroxymethyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 477.5 Retention time: 8.45 min. | 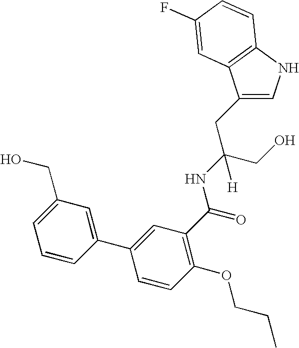 | | | |
| 289 | 3′-Chloro-4-propoxybiphenyl- 3,4′-dicarboxylic acid 3-{[2-(5-fluoro- 1H-indol-3-yl)-1-hydroxy- methylethyl]amide}4′- methylamide; N-[2-(5-Fluoro-1H-indol- 3-yl)-1-hydroxy- methylethyl]-5-iodo-2- propoxybenzamide and 3-Chloro-4-(methyl- aminocarbonyl)- phenylboronic acid | 135 | | |
| # Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 539 Retention time: 8.2 min. | 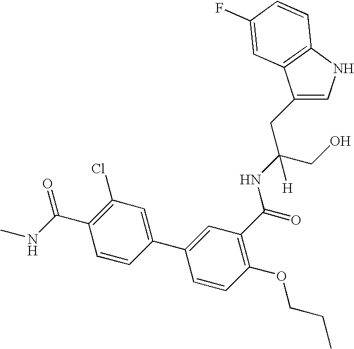 | | | |
| 290 | N-[(R)-2-Hydroxy-1-(1-methyl- 1H-indol-3-ylmethyl)ethyl]-5-(5- hydroxymethylthio- phen-2-yl)-2- propoxybenzamide; N-[(R)-1-Hydroxymethyl- 2-(1-methyl-1H-indol-3- yl)ethyl]-5-iodo-2- propoxybenzamide and 5-(Hydroxymethyl)- thiophene-2- boronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 479.6 Retention time: 8.87 min. | 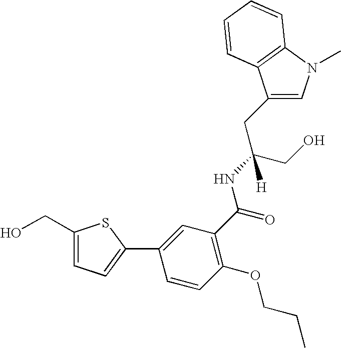 | | | |
| 291 | 3′-Chloro-4-propoxybiphenyl- 3,4′-dicarboxylic acid 3-{[(R)-2-hydroxy-1- (1-methyl-1H-indol-3- ylmethyl)ethyl]amide}4′-methylamide; N-[(R)-1-Hydroxymethyl- 2-(1-methyl-1H-indol- 3-yl)ethyl]-5-iodo-2- propoxybenzamide and 3-Chloro-5-(methyl- carbamoyl)- phenylboronic acid | 135 | | |
| # Column Purospher Star RP C18 4.6 ×]125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 535 Retention time: 8.82 min. | 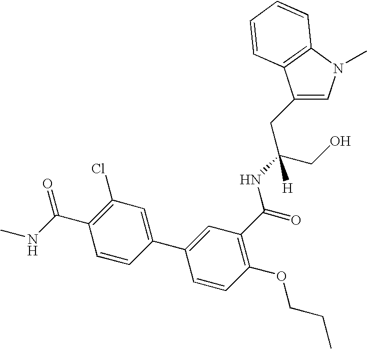 | | | |
| 292 | 4′-Hydroxymethyl-4- propoxybiphenyl-3- carboxylic acid [2-(5- fluoro-1H-indol-3-yl)- 1-hydroxymethylethyl]amide; N-[2-(5-Fluoro-1H-indol-3-yl)-1- hydroxymethylethyl]-5-iodo-2- propoxybenzamide and 4-Hydroxymethyl- phenylboronic acid | 135 | Column Purospher Star RP C18 4.6 × 125 5 μm; | |
| # detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 477.5 Retention time: 8.24 min. | 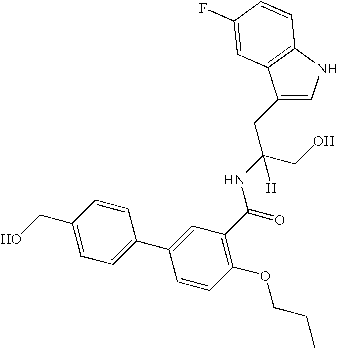 | | | |
| 293 | 4-Propoxybiphenyl-3,4′- dicarboxylic acid 3-{[2- (5-fluoro-1H-indol-3-yl)- 1-hydroxymethylethyl]amide}4′-methylamide; N-[2-(5-Fluoro-1H-indol-3-yl)- 1-hydroxymethylethyl]- 5-iodo-2-propoxy- benzamide and 4-(Methylaminocarbonyl)- phenylboronic acid | 135 | | |
| # Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 504.6 Retention time: 7.97 min. | 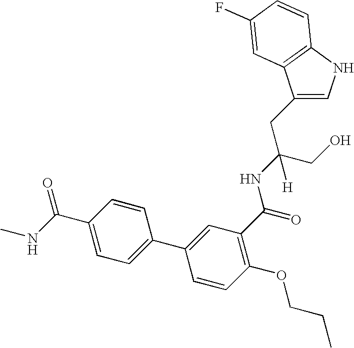 | | | |
| 294 | 5′-Fluoro-4-propoxybiphenyl- 3,3′-dicarboxylic acid 3-{[2-(5- fluoro-1H-indol-3-yl)-1- hydroxymethylethyl]amide}3′-methylamide; N-[2-(5-Fluoro-1H-indol-3- yl)-1-hydroxy- methylethyl]-5-iodo-2- propoxybenzamide and 3-Fluoro-5-(methyl- carbamoyl)- phenylboronic acid | 135 | | |
| # Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 522.6 Retention time: 8.62 min. | 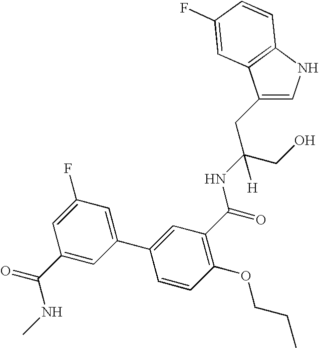 | | | |
| 295 | 4-Ethoxy-3′-fluoro-N-[(R)-1- (hydroxymethyl)- 2-(1H-indol-3- yl)ethyl]-4′- methoxy[1,1′- biphenyl]-3-carboxamide; D-Tryptophanol and 4-Ethoxy-3′-fluoro-4′- methoxy[1,1′- biphenyl]-3- carboxylic acid | 39 | (DMSO-d₆): 10.82 s (1H); 8.39 d (J=8.1Hz, 1H); 8.12 d (J=2.5 Hz, 1H); | |
| # 7.75 dd (J=8.7 Hz/2.5 Hz, 1H); 7.70 d (J=8.0 Hz, 1H); 7.51 dd (J=12.9 Hz/ 2.2 Hz, 1H); 7.43 d (J=8.8 Hz, 1H); 7.34 d (J=8.0 Hz, 1H); 7.24 dd (J=8.8 Hz/ 8.8 Hz, 1H); 7.19 d (J=8.7 Hz, 1H); 7.16 d (J=2.3 Hz, 1H); 7.06 dd (J=8.0 Hz/ 7.0 Hz, 1H); 6.97 dd (J=8.0 Hz/7.0 Hz, 1H); 4.93 m (1H); 4.26 m (1H); 4.15 m (2H); 3.88 s (3H); 3.50 m (1H); 3.45 m (1H); | | | | |
| # 3.00 dd (J=14.4 Hz/7.6 Hz, 1H); 2.97 dd (J=14.4 Hz/5.8 Hz, 1H); 1.31 t (J=7.0 Hz, 3H). | 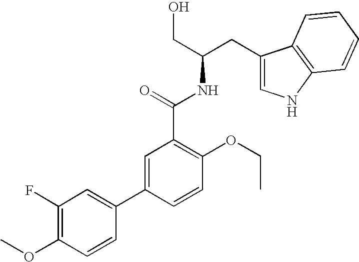 | | | |
| 296 | 4-Ethoxy-N-[(R)-1- (hydroxymethyl)-2- (1H-indol-3- yl)ethyl]-3′-methoxy[1, 1′-biphenyl]-3- carboxamide; D-Tryptophanol and 4-Ethoxy-3′-methoxy[1, 1′-biphenyl]- 3-carboxylic acid | 39 | (DMSO-d₆): 10.82 s (1H); 8.40 d (J=8.1Hz, 1H); | |
| # 8.16 d (J=2.5 Hz, 1H); 7.77 dd (J=8.6 Hz/2.5 Hz, 1H); 7.70 d (J=8.0 Hz, 1H); 7.38 dd (J=8.3 Hz/ 7.8 Hz, 1H); 7.34 d (J=8.0 Hz, 1H); 7.21 d (J=8.6 Hz, 1H); 7.19d (J=7.8 Hz, 1H); 7.17 d (J=2.3 Hz, 1H); 7.14 dd(J=2.5 Hz/ 1.8 Hz, 1H); 7.06 dd (J=8.0 Hz/7.0 Hz, 1H); 6.97 dd (J=8.0 Hz/7.0 Hz, 1H); 6.92 dd (J=8.3 Hz/ 2.5 Hz, 1H); | | | | |
| # 4.93 m (1H); 4.27 m (1H); 4.16 m (2H); 3.83 s (3H); 3.50 m (1H); 3.46 m (1H); 3.01 dd (J=14.4 Hz/7.3 Hz, 1H); 2.96 dd (J=14.4 Hz/6.3 Hz, 1H); 1.32t(J=7.0 Hz, 3H). | 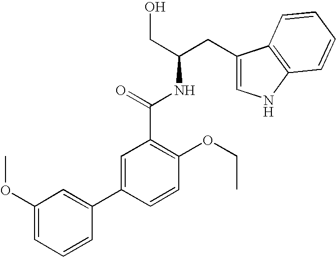 | | | |
| 297 | 4-Ethoxy-N-[(R)-1- (hydroxymethyl)-2- (1H-indol-3- yl)ethyl]-N′-methyl[1,1′- biphenyl]-3,3′- dicarboxamide; D-Tryptophanol and 4-Ethoxy-3′- [(methylamino)- carbonyl][1,1′- biphenyl]-3- carboxylic acid | 39 | (DMSO-d₆): 10.82 s (1H); | |
| # 8.61 q (J=4.6 Hz, 1H); 8.41 d (J=8.1 Hz, 1H); 8.25 d (J=2.5 Hz, 1H); 8.10 s (1H); 7.84dd (J=8.6 Hz/2.5 Hz, 1H); 7.80 d (J=7.8 Hz, 1H); 7.78d (J=7.6 Hz, 1H); 7.71 d (J=8.0 Hz, 1H); 7.54 dd (J=7.8 Hz/7.6 Hz, 1H); 7.33 d (J=8.0 Hz, 1H); 7.25 d (J=8.6 Hz, 1H); 7.17 d (J=2.3 Hz, 1H); 7.06 dd (J=8.0 Hz/7.0 Hz, 1H); | | | | |
| # 6.97 dd (J=8.0 Hz/7.0 Hz, 1H); 4.94 m (1H); 4.28 m (1H); 4.17 m (2H); 3.51 m (1H); 3.46 m (1H); 3.01 dd (J=14.4 Hz/7.3 Hz, 1H); 2.98 dd (J=14.4 Hz/6.1 Hz, 1H); 2.82 d (J=4.6 Hz, 3H), 1.33 t (J=7.0 Hz, 3H). | 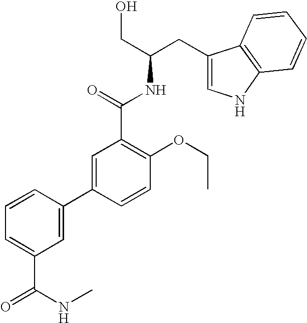 | | | |
| 298 | 4-Ethoxy-N-[(R)-1- (hydroxymethyl)-2- (1H-indol-3-yl)ethyl]- 3′,4′,5′-trimethoxy[1,1′- biphenyl]-3-carboxamide; D-Tryptophanol and 4-Ethoxy-3′,4′,5′-trimethoxy[1,1′- biphenyl]-3- carboxylic acid | 39 | (DMSO-d₆): 10.82 s (1H); 8.41 d (J=7.9 Hz, 1H); | |
| # 8.14 d (J=2.5 Hz, 1H); 7.78 dd (J=8.7 Hz/2.5 Hz, 1H); 7.70 d (J=8.0 Hz, 1H); 7.33 d (J=8.0 Hz, 1H); 7.20 d (J=8.7 Hz, 1H); 7.17 s (1H); 7.06 dd (J=8.0 Hz/7.0 Hz, 1H); 6.97 dd (J=8.0 Hz/7.0 Hz, 1H); 6.86 s (2H); 4.93 m (1H); 4.26 m (1H); 4.16 m (2H); 3.86 s (6H); 3.69 s (3H); 3.50 m (1H); 3.45 m (1H); 3.01 dd (J=14.2 Hz/ 7.0 Hz, 1H); | | | | |
| # 2.97 dd (J=14.2 Hz/6.2 Hz, 1H); 1.32 t (J=7.0 Hz, 3H). | 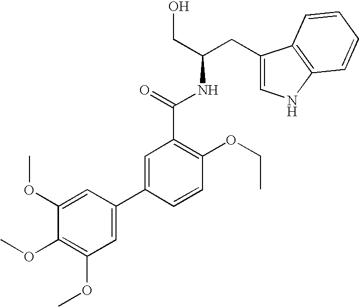 | | | |
| 299 | 4-Ethoxy-N-[(R)-1- (hydroxymethyl)-2- (1H-indol-3- yl)ethyl]-3′,4′- dimethoxy[1,1′- biphenyl]-3- carboxamide; D-Tryptophanol and 4-Ethoxy-3′,4′- dimethoxy[1,1′- biphenyl]-3- carboxylic acid | 39 | (DMSO-d₆): 10.82 s (1H); 8.41 d (J=8.1 Hz, 1H); | |
| # 8.13 d (J=2.5 Hz, 1H); 7.74 dd (J=8.6 Hz/2.5 Hz, 1H); 7.70 d (J=8.0 Hz, 1H); 7.33 d (J=8.0 Hz, 1H); 7.18 d (J=8.6 Hz, 1H); 7.16 s (2H); 7.15 dd (J=8.1 Hz/2.3 Hz, 1H); 7.06 dd (J=8.0 Hz/7.0 Hz, 1H); 7.03 d (J=8.1 Hz, 1H); 6.97 dd (J=8.0 Hz/ 7.0 Hz, 1H); 4.93 m (1H); 4.27 m (1H); 4.15 m (2H); 3.85 s (3H); 3.79 s (3H); 3.50 m (1H); | | | | |
| # 3.45 m (1H); 3.01 dd (J=14.2 Hz/7.3 Hz, 1H); 2.97 dd (J=14.2 Hz/5.8 Hz, 1H); | | | | |
| # 1.32 t (J=7.0 Hz, 3H). | 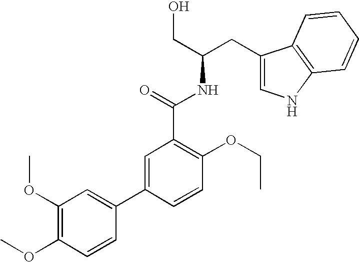 | | | |
| 300 | 4-Ethoxy-N-[(R)-1- (hydroxymethyl)-2- (1H-indol-3- ylethyl]-3′-(1- methylethyl)[1,1′- biphenyl]-3- carboxamide; D-Tryptophanol and 4-Ethoxy-3′-(1-methyl- ethyl)[1,1′-biphenyl]- 3-carboxylic acid | 39 | (CDCl₃): 8.14 s (1H); 8.52 m (1H); 8.51 d (J=2.5 Hz, | |
| # 1H); 7.71 d (J=8.0 Hz, 1H); 7.66 dd (J=8.6 Hz/ 2.5 Hz, 1H); 7.48 s (1H); 7.42 d (J=7.8 Hz, 1H); 7.36 d (J=8.0 Hz, 1H); 7.35 dd (J=7.8 Hz/7.6 Hz, 1H); 7.20 d (J=7.6 Hz, 1H); 7.19 dd (J=8.0 Hz/ 7.0 Hz, 1H); 7.12 dd (J=8.0 Hz/7.0 Hz, 1H); 7.11 d (J=2.3 Hz, 1H); 6.97 d (J=8.6 Hz, 1H); 4.58 | | | | |
| # m (1H); 4.06 m (2H); 3.84 d (J=10.9 Hz, 1H); 3.77 dd (J=10.9 Hz, 1H); 3.77 dd (J=10.9 Hz/5.1 Hz, 1H); 3.17 dd (J=15.2 Hz /6.8 Hz, 1H); 3.14 dd (J=15.2 Hz/ 6.8 Hz, 1H); 2.97 sept (J=6.8 Hz, 1H); 1.30 d (J=6.8 Hz, 6H); 1.26 t (J=7.0 Hz, 3H). | 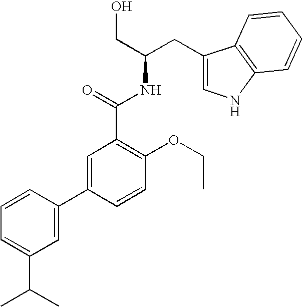 | | | |
| 301 | N-[(R)-1-(Hydroxy- methyl)-2-(1H- indol-3-yl)ethyl]-3′,4′,5′- trimethoxy-4-propoxy[1, 1′-biphenyl]-3- carboxamide; D-Tryptophanol and 3′,4′,5′-Trimethoxy-4- propoxy[1,1′- biphenyl]-3- carboxylic acid | 39 | (CDCl₃): 8.49 d (J=7.5 | |
| # Hz, 1H); 8.47 d (J=2.6 Hz, 1H); 8.12 s (1H); 7.71 d (J=8.0 Hz, H); 7.61 dd (J=8.7 Hz/2.6 Hz, 1H); 7.36 d (J=8.0 Hz, 1H); 7.20 dd (J=8.0 Hz/7.0 Hz, 1H); 7.11 dd (J=8.0 Hz/7.0 Hz, 1H); 7.11 s (1H); 6.98 d (J=8.7 Hz, 1H); 6.79 s (2H); 4.60 m (1H); 3.97 m (2H); 3.92 s (6H); 3.89 s (3H); 3.81 m (2H); 3.15 m (2H); | | | | |
| # 1.66 m (2H); 0.95 t (J=7.4 Hz, 3H). | 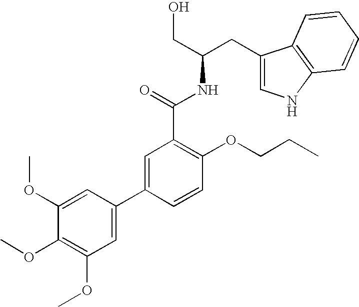 | | | |
| 302 | N-[(R)-1-(Hydroxy- methyl)-2-(1H- indol-3-yl)ethyl]-3′,4′- dimethoxy-4- propoxy[1,1′-biphenyl]-3- carboxamide; D-Tryptophanol and 3′,4′-Dimethoxy-4- propoxy[1,1′- biphenyl]-3- carboxylic acid | 39 | (CDCl₃): 8.49 d (J=7.4 | |
| # Hz, 1H); 8.47 d (J=2.5 Hz, 1H); 8.11 s (1H); 7.71 d (J=8.0 Hz, 1H); 7.62 dd (J=8.7 Hz/2.5 Hz, 1H); 7.36 d (J=8.0 Hz, 1H); 7.19 dd (J=8.0 Hz/7.0 Hz, 1H); 7.12 m (4H); 6.97 d (J=8.7 Hz, 1H); 6.93 d (J=8.3 Hz, 1H); 4.59 m (1H); 3.97 m (2H); 3.95 s (3H); 3.92 s (3H); 3.81 m (2H); 3.18 dd (J=15.1 Hz/6.8 Hz, 1H); 3.13 dd(J= | | | | |
| # 15.1 Hz/7.9 Hz, 1H); 1.65 m (2H); 0.94 t (J=7.4 Hz, 3H). | 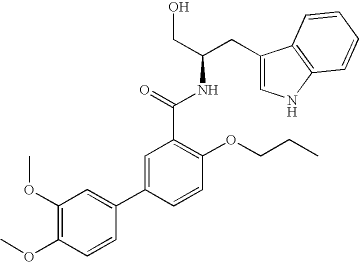 | | | |
| 303 | N-[(R)-1-(Hydroxy- methyl)-2-(1H- indol-3-yl)ethyl]-3′- methoxy-4- propoxy[1,1′-biphenyl]-3- carboxamide; D-Tryptophanol and 3′-Methoxy-4- propoxy[1,1′- biphenyl]-3- carboxylic acid | 39 | (CDCl₃): 8.51 d (J=2.6 Hz, 1H); 8.46 d (J=7.5 Hz, | |
| # 1H); 8.09 s (1H); 7.71 d (J=8.0 Hz, H); 7.65 dd (J=8.7 Hz/2.6 Hz, 1H); 7.36 d (J=8.0 Hz, 1H); 7.34 dd (J=7.9 Hz/7.9 Hz, 1H); 7.20 d (J=7.9 Hz, H); 7.19 dd (J=8.0 Hz/7.0 Hz, 1H); 7.14 s (1H); 7.11 dd (J=8.0 Hz/7.0 Hz, 1H); 7.11 s (1H); 6.98 d (J=8.7 Hz, 1H); 6.88 dd (J=7.9 Hz/ 2.5 Hz, 1H); 4.59 m (1H); 3.96 m (2H); 3.86 s (3H); 3.80 m (2H); | | | | |
| # 3.16 m (2H); 1.65 m (2H); 0.93 t (J=7.4 Hz, 3H). | 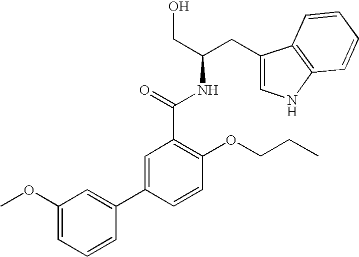 | | | |
| 304 | N-[(R)-1-(Hydroxy- methyl)-2-(1H- indol-3-yl)ethyl]-N′- methyl-4-propoxy[1,1′- biphenyl]-3,3′- dicarboxamide; D-Tryptophanoland 3′-[(Methylamino)- carbonyl]-4- propoxy[1,1′- biphenyl]-3- carboxylic acid | 39 | (CDCl₃): 8.47 d (J=2.5 | |
| # Hz, 1H); 8.42 d (J=7.4 Hz, 1H); 8.21 s (1H); 7.97 dd (J=1.7 Hz/1.7 Hz, 1H); 7.72 m (3H); 7.66 dd (J=8.7 Hz/ 2.5 Hz, 1H); 7.47 dd (J=7.7 Hz/7.5 Hz, 1H); 7.36 d (J=8.0 Hz, 1H); 7.19 dd (J=8.0 Hz/7.0 Hz, 1H); 7.11 dd (J=8.0 Hz/7.0 Hz, 1H); 6.97 d (J=8.7 Hz, 1H); 6.37 m (1H); 4.58 m (1H); 3.95 m (2H); 3.80 m (2H); 3.16 dd (J=15.1 Hz/ 6.8 Hz, 1H); | | | | |
| # 3.13 dd (J=15.1 Hz/7.9 Hz, 1H); 3.04 d (J=4.9 Hz, 3H); 1.65 m (2H); 0.93 t (J=7.4 Hz, 3H). | 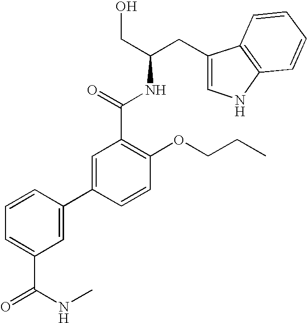 | | | |
| 305 | 4,3′,4′,5′-Tetra- methoxybiphenyl- 3-carboxylic acid [(R)-1-hydroxymethyl- 2-(1H-indol-3- yl)ethyl]amide; (D)-Tryptophanol and 4-Methoxy-3′,4′,5′-tri- methoxybiphenyl-3- carboxylic acid | 39 | (DMSO-d₆): 10.85 s (1H); 8.19 d (J=7.8 Hz, 1H); | |
| # 8.05 d (J=2.3 Hz, 1H); 7.76 dd (J=8.6 Hz, J=2.3 Hz, 1H); 7.69 d (J=7.8 Hz, 1H); 7.33 d (J=7.8 Hz, 1H); 7.18-7.20 m (2H); 7.06 t (J=7.2 Hz, 1H); 6.97 t (J=7.4 Hz, 1H); 6.83 s (2H); 4.93 t (J=5.2 Hz, 1H); 4.20-4.23 m (lH); 3.85 s (6H); 3.84 s (3H); 3.68 s (3H); | | | | |
| # 3.40-3.56 m (2H);2.95-3.05 m (2H). | 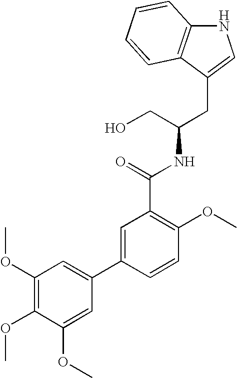 | | | |
| 306 | 4,3′,4′-Trimethoxy- biphenyl-3- carboxylic acid [(R)-1-hydroxy- methyl-2-(1H-indol-3- yl)ethyl]amide; (D)-Tryptophanol and 4,3′,4′-Trimethoxy- biphenyl-3- carboxylic acid | 39 | (DMSO-d₆): 10.85 s (1H); 8.20 d (J=7.8 Hz, 1H); 8.04 d (J=2.3 Hz, 1H); | |
| # 7.73 dd (J=8.6 Hz, J=2.3 Hz, 1H); 7.69 d (J=7.8 Hz, 1H); 7.34 d (J=8.2 Hz, 1H); 7.12-7.20 m (4H); 7.06 t (J=7.6 Hz, 1H); 7.02 d (J=8.2 Hz, 1H); 6.98 t (J=7.4 Hz, 1H); 4.94 t (J=5.1 Hz, 1H); 4.21-4.29 m (1H); 3.84 s (3H); 3.83 s (3H); 3.78 s (3H); 3.40-3.56 m (2H); 2.96-3.05 m (2H). | 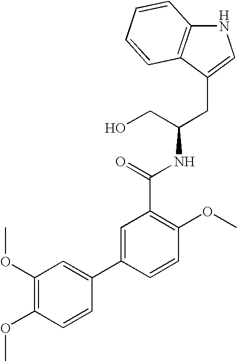 | | | |
| 307 | 3′-Fluoro-4,4′- dimethoxybiphenyl- 3-carboxylic acid [(R)-2-hydroxy- 1-(1H-indol- 3-ylmethyl)ethyl]amide; (D)-Tryptophanol and 3′-Fluoro-4,4′- dimethoxybiphenyl- 3-carboxylic acid | 39 | (DMSO-d₆): 10.85 s (1H); 8.18 d (J=7.8 Hz, 1H); 8.01 d (J=2.3 Hz, 1H); | |
| # 7.73 dd (J=8.7 Hz, J=2.3 Hz, 1H); 7.69 d (J=7.8 Hz, 1H); 7.49 dd (J=12.9 Hz, J=1.9 Hz, 1H); 7.40 d (J=8.6 Hz, 1H); 7.34 d (J=8.2 Hz, 1H); 7.23 t (J=8.8 Hz, 1H); 7.17-7.19 m (2H); 7.06 t (J=7.4 Hz, 1H); 6.98 t (J=7.4 Hz, 1H); 4.93 t (J=5.2 Hz, 1H); 4.20-4.28 m (1H); 3.87 s (3H); 3.83 s (3H); 3.40-3.56 m (2H); 2.95-3.05 m (2H). | 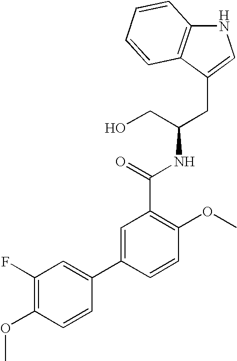 | | | |
| 308 | 4,3′-Dimethoxy- biphenyl-3- carboxylic acid [(R)-1-hydroxy- methyl-2-(1H-indol-3- yl)ethyl]amide; (D)-Tryptophanol and 4,3′-Dimethoxy- biphenyl-3- carboxylic acid | 39 | (DMSO-d₆): 10.85 s (1H); 8.19 d (J=8.2 Hz, 1H); 8.06 d (J=2.7 Hz, 1H); | |
| # 7.76 dd (J=8.8 Hz, J=2.5 Hz, 1H); 7.69 d (J=7.8 Hz, 1H); 7.36 t (J=8.0 Hz, 1H); 7.34 d (J=8.2 Hz, 1H); 7.16-7.21 m (3H); 7.12 (1H); 7.06 t (J=7.4 Hz, 1H); 6.98 t (J=7.4 Hz, 1H); 6.91 dd (J=8.2 Hz, J=2.3 Hz, 1H); 4.93 t (J=5.2 Hz, 1H); 4.21-4.29 m (1H); 3.83 s (3H); 3.82 s (3H); 3.41-3.56 m (2H); 2.95-3.06 m (2H). | 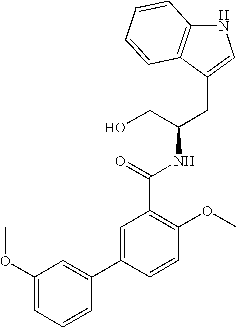 | | | |
| 309 | 5-Benzo[1,3]dioxol- 5-yl-N-[(R)- 1-hydroxymethyl- 2-(1H-indol-3- yl)ethyl]-2- methoxybenzamide; (D)-Tryptophanol and 5-Benzo[1,3]dioxol- 5-yl-2-methoxy- benzoic acid | 39 | (DMSO-d₆): 10.84 s (1H); 8.17 d (J=7.8 Hz, 1H); 8.04 d (J=2.3 Hz, 1H); | |
| # 7.97 d (J=2.3 Hz, 1H); 7.66-7.70 m (1H); 7.34 d (J=7.8 Hz, 1H); 7.15-7.19 m (3H); 7.04-7.08 m (2H); 6.96-6.99 m (2H); 6.05 s (2H); 4.93 t (J=5.2 Hz, 1H); 4.20-4.28 m (1H); 3.82 s (3H); 3.40-3.56 m (2H); 2.96-3.05 m (2H). | 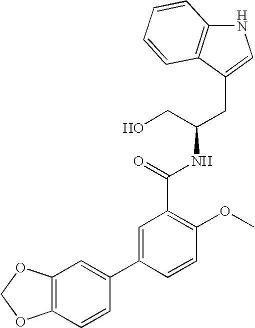 | | | |
| 310 | 3′,4′-Difluoro-4,5′- dimethoxybiphenyl- 3-carboxylic acid [(R)-2-hydroxy-1- (1H-indol-3- ylmethyl)ethyl]amide; (D)-Tryptophanol and 3′,4′-Difluoro-4,5′- dimethoxy- biphenyl-3- carboxylic acid | 39 | (DMSO-d₆): 10.84 s (1H); | |
| # 8.17 d (J=7.8 Hz, 1H); 8.04 d (J=1.9 Hz, 1H); 7.80 dd (J=8.8 Hz, J=2.3 Hz, 1H); 7.69 d (J=7.8 Hz, 1H); 7.33 d (J=7.8 Hz, 1H); 7.19-7.27 m (4H); 7.06 t (J=7.4 Hz, 1H); 6.97 t (J=7.4 Hz, 1H); 4.93 t (J=4.8 Hz, 1H); 4.20-4.28 m (1H); 3.97 s (3H); 3.84 s (3H); 3.40-3.56 m (2H); 2.95-3.05 m (2H). | 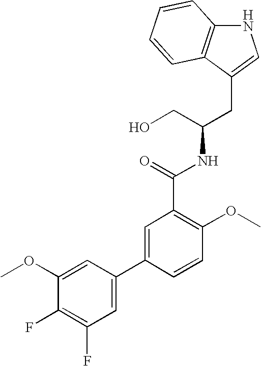 | | | |
| 311 | 4-Isopropoxy-3′- methoxybiphenyl- 3-carboxylic acid [(R)-1-hydroxy- methyl-2-(1H- indol-3-yl)ethyl]amide; (D)-Tryptophanol and 4-Isopropoxy-3′- methoxybiphenyl- 3-carboxylic acid | 39 | (DMSO-d₆) 10.82 s (1H); 8.49 d (J=8.20 Hz, 1H); 8.19 d (J=2.3 Hz, 1H); | |
| # 7.75 dd (J=8.6 Hz, H=2.7 Hz, 1H); 7.71 d (J=7.8 Hz, 1H); 7.37 t (J=8 Hz, 1H); 7.33 d (J=8.2 Hz, 1H); 7.24 d (J=8.6 Hz, 1H); 7.19 d (J=7.8 Hz, 1H); 7.14-7.16 m (2H); 7.06 t (J=7.6 Hz, 1H); 6.97 t (J=7.2 Hz, 1H); 6.92 dd (J=8.2 Hz, J=2 Hz, 1H); 4.97 t (J=5.1 Hz, 1H); 4.78-4.84 m (1H); 4.23-4.30 m (1H); 3.82 s (3H); 3.42-3.54 | 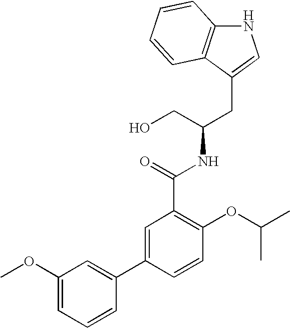 | | | |
| 312 | 5-Benzo[1,3]dioxol- 5-yl-N-[(R)-1- hydroxymethyl-2- (1H-indol-3-yl)ethyl]-2- isopropoxybenzamide; (D)-Tryptophanol and 5-Benzo[1,3]dioxol-5- yl-2-isopropoxy- benzoic acid | 39 | (DMSO-d₆): 10.82 s (1H); 8.48 d (J=8.20 Hz, 1H); | |
| # 8.10 d(J=2.3 Hz, 1H); 7.71 d (J=7.8 Hz, 1H); 7.67 dd (J=8.6 Hz, J=1.9 Hz, 1H); 7.33 d (J=8.2 Hz, 1H); 7.19-7.21 m (2H); 7.15 (1H); 7.04-7.10 m (2H); 6.95-6.99 m (2H); 6.05 s (2H); 4.97 t (J=5.1 Hz, 1H); 4.75-4.81 m (1H); 4.22-4.29 m (1H); 3.42-3.54 m (2H); 2.94-3.04 m (2H); 1.28 d (J=5.6 Hz, 3H); 1.27 d (J=5.6 Hz, | 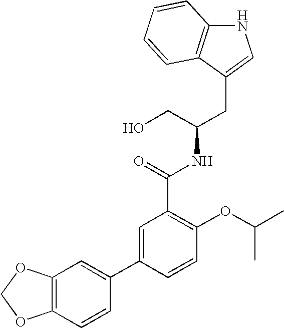 | | | |
| 313 | 4-Isopropoxy-3′,4′,5′- trimethoxybiphenyl- 3-carboxylic acid [(R)-1-hydroxy- methyl-2-(1H-indol-3- yl)ethyl]amide; (D)-Tryptophanol and 4-Isopropoxy-3′,4′,5′- trimethoxybiphenyl- 3-carboxylic acid | 39 | (DMSO-d₆): 10.82 s (1H); 8.49 d (J=8.20 Hz, 1H); | |
| # 8.16 d (J=2.3 Hz, 1H); 7.75 dd (J=8.6 Hz, J=2.3 Hz, 1H); 7.71 d (J=7.8 Hz, 1H); 7.33 d (J=7.8 Hz, 1H); 7.24 d (J=8.6 Hz, 1H); 7.15 (1H); 7.06 t (J=7.6 Hz, 1H); 6.97 t (J=7.4 Hz, 1H); 6.85 s (2H); 4.97 t (J=4.7 Hz, 1H); 4.78-4.84 m (1H); 4.23-4.30 m (1H); 3.86 s (3H); 3.69 s (3H); 3.42-3.54 m (2H); 2.95-3.04 m (2H); 1.28 d (J=5.6 Hz, 3H); 1.27 d (J=5.6 Hz, 3H). | 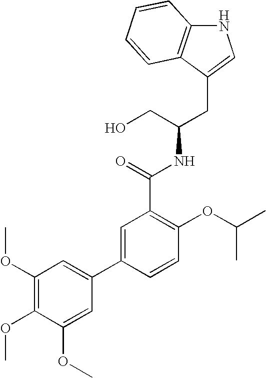 | | | |
| 314 | 3′-Fluoro-4-iso- propoxy-4′- methoxybiphenyl- 3-carboxylic acid [(R)-2-hydroxy-1-(1H-indol- 3-ylmethyl)ethyl]amide; (D)-Tryptophanol and 3′-Fluoro-4-isopropoxy- 4′-methoxy- biphenyl-3- carboxylic acid | 39 | (DMSO-d₆): 10.83 s (1H); 8.48 d (J=8.20 Hz, 1H); 8.15 d (J=2.3 Hz, 1H); | |
| # 7.71 d (J=7.4 Hz, 1H); 7.50 dd (J=12 Hz, J=1.9 Hz, 1H); 7.42 d (J=8.6 Hz, 1H); 7.33 d (J=8.2 Hz, 1H); 7.24 t (J=8.2 Hz, 1H); 7.22 d (J=8.8 Hz, 1H); 7.16 (1H); 7.06 t (J=7.4 Hz, 1H); 6.97 t (J=7.4 Hz, 1H); 4.98 t (J=5.1 Hz, 1H); 4.77-4.83 m (1H); 4.23-4.30 m (1H); 3.87 s (3H); 3.42-3.55 m (2H); | | | | |
| # 2.95-3.05 m (2H); 1.28 d (J=5.6 Hz, 3H); 1.27 d (J=5.6 Hz, 3H). | 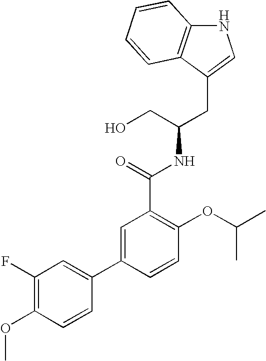 | | | |
| 315 | 4-Isopropoxy-3′,4′- dimethoxybiphenyl- 3-carboxylic acid [(R)-1-hydroxymethyl-2- (1H-indol-3-yl)ethyl]amide; (D)-Tryptophanol and 4-Isopropoxy-3′,4′- dimethoxybiphenyl- 3-carboxylic acid | 39 | (DMSO-d₆): 10.82 s (1H) 8.50 d (J=7.8 Hz, 1H); 8.16 d (J=2.3 Hz, 1H); 7.71 dd (J=8.6 Hz, J=2.7 | |
| # Hz, 1H); 7.34 d (J=8.2 Hz, 1H); 7.22 d (J=8.9 Hz, 1H); 7.12-7.16 m (3H); 7.06 t (J=7.4 Hz, 1H); 7.02 d (J=8.6 Hz, 1H); 6.97 t (J=7.4 Hz, 1H); 4.97 t (J=5.1 Hz, 1H); 4.76-4.82 m (1H); 4.23-4.30 m (1H); 3.84 s (3H); 3.79 s (3H); 3.42-3.54 m (2H); 2.94-3.04 m (2H); 1.28 d (J=5.6 Hz, 3H); d (J=5.6 Hz, 3H); | 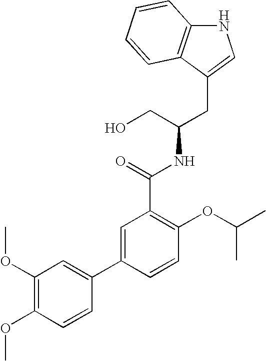 | | | |
| 316 | 4-Isopropoxy-3′- methylbiphenyl- 3-carboxylic acid [(R)-1- hydroxymethyl-2- (1H-indol-3- yl)ethyl]amide; (D)-Tryptophanol and 4-Isopropoxy-3′-methyl- biphenyl-3- carboxylic acid | 39 | (DMSO-d₆): 10.82 s (1H); 8.50 d (J=8.2 Hz, 1H); | |
| # 8.20 d (J=2.3 Hz, 1H); 7.72 dd (J=8.6 Hz, J=2.7 Hz, 1H); 7.45 (1H); 7.41 d (J=7.8 Hz, 1H); 7.31-7.35 m (2H); 7.24 d (J=8.6 Hz, 1H); 7.14-7.16 m (2H); 7.06 t (J=7.4 Hz, 1H); 6.97 t (J=7 Hz, 1H); 4.97 t (J=5.1 Hz, 1H); 4.77-4.83 m (1H); 4.24-4.31 m (1H); 3.43-3.55 m (2H); 2.95-3.05 m (2H); 2.38 s (3H); 1.28 d (J= | | | | |
| # 5.6 Hz, 3H); 1.27 d(J=5.6 Hz, 3H). | 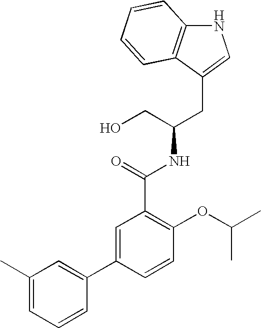 | | | |
| 317 | 4′-Fluoro-4-iso- propoxy-3′- methylbiphenyl-3- carboxylic acid [(R)-2-hydroxy-1- (1H-indol-3- yl)methyl)ethyl]amide; (D)-Tryptophanol and 4′-Fluoro-4-iso- propoxy-3′-methyl- biphenyl-3- carboxylic acid | 39 | (DMSO-d₆): 10.82 s (1H); 8.50 d (J=8.2 Hz, 1H); | |
| # 8.17 d (J=2.3 Hz, 1H); 7.74-7.70 m (2H); 7.56 d (J=7 Hz, 1H); 7.45-7.48 (1H); 7.34 d (J=8.2 Hz, 1H); 7.20-7.25 m (2H); 7.16 (1H); 7.06 t (J=7.4 Hz, 1H); 6.97 t (J=7.4 Hz, 1H); 4.97 t (J=5.1Hz, 1H); 4.77-4.83 m (1H); 4.23-4.31 m (1H); 3.43-3.55 m (2H); 2.95-3.05 m (2H); 2.31 s (3H); 1.28 d | | | | |
| # (J=5.6 Hz, 3H); 1.27 d (J=5.6 Hz, 3H). | 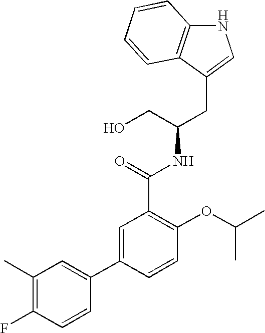 | | | |
| 318 | 3′,4′-Difluoro- 4-isopropoxy-5′- methoxybiphenyl-3- carboxylic acid [(R)-2-hydroxy-1- (1H-indol-3- yl)methyl)ethyl]amide; (D)-Tryptophanol and 3′,4′-Difluoro-4-iso- propoxy-5′-methoxy- biphenyl-3- carboxylic acid | 39 | (DMSO-d₆): 10.82 s (1H); | |
| # 8.48 d (J=7.8 Hz, 1H); 8.18 d (J=1.6 Hz, 1H); 7.78 dd (J=8.74 Hz, J=1.8 Hz, 1H); 7.72 d (J=7.8 Hz, 1H); 7.33 d (J=8.2 Hz, 1H); 7.28-7.23 m (2H); 7.20 d (J=6.62 Hz, 1H); 7.16 (1H); 7.06 t (J=7.4 Hz, 1H); 6.97 t (J=7.4 Hz, 1H); 4.97 t (J=5.1 Hz, 1H); 4.80-4.86 m (1H); 4.23-4.30 m (1H); 3.98 s (3H); 3.42- | | | | |
| # 3.55 m (2H); 2.95-3.04 m (2H); 1.28 d (J=5.6 Hz, 3H); 1.27 d (J=5.6 Hz, 3H). | 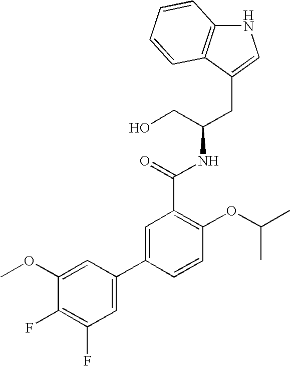 | | | |
| 319 | 4,3′,4′,5′-Tetra- methoxy-5- methylbiphenyl-3- carboxylic acid [(R)-1-hydroxy- methyl-2- (1H-indol-3-yl)ethyl]amide; (D)-Tryptophanol and 4,3′,4′,5′-Tetramethoxy- 5-methylbiphenyl-3- carboxylic acid | 39 | (DMSO-d₆): 10.81 s (1H); | |
| # 8.26 d (J=7.8 Hz, 1H); 7.69 d (J=7.8 Hz, 1H); 7.63-7.65 m (2H); 7.32 d (J=7.8 Hz, 1H); 7.18 (1H); 7.05 t (J=7.2 Hz, 1H); 6.97 t (J=7.4 Hz, 1H); 6.84 s (2H); 4.90 t (J=5.2 Hz, 1H); 4.22-4.30 m (1H); 3.86 s (6H); 3.69 s (3H); 3.62 s (3H); 3.44-3.58 m (2H); 3.03. 2.96 AB (J₁= | | | | |
| # 14.4 Hz, J₂=6.9 Hz, 2H); 2.31 s (3H). | 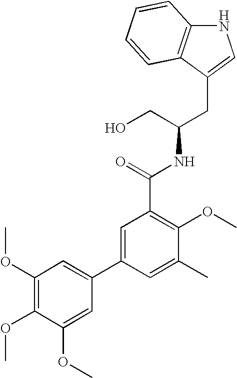 | | | |
| 320 | 4,3,4′-Trimethoxy-5- methylbiphenyl-3- carboxylic acid [(R)-1-hydroxy- methyl-2-(1H- indol-3-yl)ethyl]amide; (D)-Tryptophanol and 4,3′,4′-Trimethoxy-5- methylbiphenyl-3- carboxylic acid | 39 | (DMSO-d₆): 10.81 s (1H); 8.25 d (J=8.2 Hz, 1H); 7.69 d (J=7.8 Hz, 1H); | |
| # 7.59-7.61 m (2H); 7.32 d (J=7.8 Hz, 1H); 7.18 (1H); 7.12-7.15 m (2H); 7.01-7.07 m (2H); 6.97 t (J=7.4 Hz, 1H); 4.89 t (J=5.2 Hz, 1H); 4.23-4.30 m (1H); 3.84 s (6H); 3.70 s (3H); 3.61 s (3H); 3.44-3.58 m (2H); 3.03. 2.95 AB (J₁=14.3 Hz, J₂=7 Hz, 2H); 2.31 s (3H). | 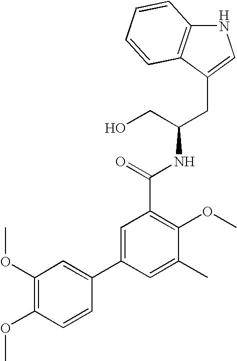 | | | |
| 321 | 3′-Fluoro-4,4′- dimethoxy-5- methylbiphenyl-3- carboxylic acid [(R)-2-hydroxy-1- (1H-indol-3- ylmethyl)ethyl]amide; (D)-Tryptophanol and 3′-Fluoro-4,4′- dimethoxy-5- methylbiphenyl-3- carboxylic acid | 39 | (DMSO-d₆): 10.81 s (1H); | |
| # 8.23 d (J=8.2 Hz, 1H); 7.69 d (J=7.8 Hz, 1H); 7.58-7.59 m (2H); 7.49 dd J=13.1 Hz, J=1.7 Hz, 1H); 7.40 d (J=8.2 Hz, 1H); 7.33 d (J=7.8 Hz, 1H); 7.23 t (J=9 Hz, 1H); 7.17 d (J=1.6 Hz, 1H); 7.06 t (J=7.4 Hz, 1H); 6.97 t (J=7.4 Hz, 1H); 4.89 t (J=5.4 Hz, 1H); 4.23-4.30 m (1H); 3.88 s (3H); 3.60 s (3H); | | | | |
| # 3.44-3.58 m (2H); 3.03, 2.95 AB (J₁=14.2 Hz, J₂=7 Hz, 2H); 2.29 s (3H). | 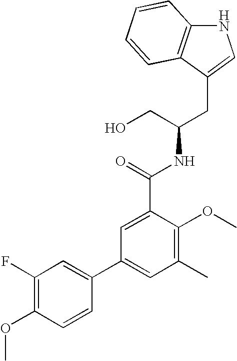 | | | |
| 322 | 5-Benzo[1,3]dioxol- 5-yl-N-[(R)- 1-hydroxymethyl-2-(1H- indol-3-yl)ethyl]-2- methoxy-3-methyl- benzamide; (D)-Tryptophanol and 5-Benzo[1,3]dioxol-5-yl-2- methoxy-3-methyl- benzoic acid | 39 | (DMSO-d₆): 10.80 s (1H); 8.21 d (J=8.2 Hz, 1H); | |
| # 7.68 d (J=7.8 Hz, 1H); 7.54 s (2H); 7.30 d (J=8.2 Hz, 1H); 7.17-7.18 m (2H); 7.04-7.08 m (1H); 6.95-6.99 m (2H); 6.06 s (2H); 4.88 t (J=5.1 Hz, 1H); 4.22-4.30 m (1H); 3.60 s (3H); 3.44-3.57 m (2H); 3.02, 2.94 AB (J₁=14.3 Hz, J₂=6.6 Hz, 2H); 2.28 s (3H). | 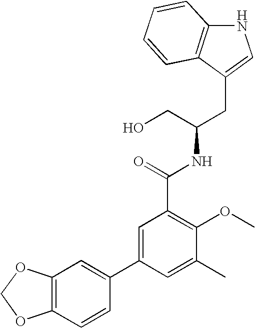 | | | |
| 323 | 4,3′-Dimethoxy-5- methylbiphenyl-3- carboxylic acid [(R)-1- hydroxymethyl-2- (1H-indol-3-yl)ethyl]amide; (D)-Tryptophanol and 4,3′-Dimethoxy-5- methylbiphenyl-3- carboxylic acid | 39 | (DMSO-d₆): 10.81 s (1H); 8.25 d (J=8.2 Hz, 1H); 7.69 d (J=7.8 Hz, 1H); 7.61-7.63 m (2H); 7.36 t (J= | |
| # 8.2 Hz, 1H); 7.33 d (J=8.2 Hz, 1H); 7.14-7.18 m (3H); 7.06 t (J=7.4 Hz, 1H); 6.97 t (J=7.4 Hz, 1H); 6.92 dd (J=8.2 Hz, J=1.9 Hz, 1H); 4.89 t (J=5.1 Hz, 1H); 4.23-4.31 m (1H); 3.82 s (3H); 3.62 s (3H); 3.45-3.58 m (2H); 3.03, 2.95 AB (J₁=14.5 Hz, J₂=6.6 Hz, 2H); 2.31 s (3H). | 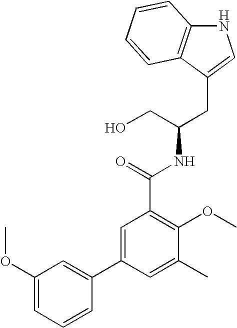 | | | |
| 324 | 4-Methoxy-5,3′- dimethylbiphenyl- 3-carboxylic acid [(R)-1-hydroxymethyl-2- (1H-indol-3-yl)ethyl]amide; (D)-Tryptophanol and 4-Methoxy-5,3′-dimethyl- biphenyl-3- carboxylic acid | 39 | (DMSO-d₆): 10.80 s (1H); 8.24 d (J=8.2 Hz, 1H); 7.69 d (J=7.8 Hz, 1H); | |
| # 7.60, 7.63 AB (J₁=14.8 Hz, J₂=1.9 Hz, 2H); 7.43 (1H); 7.39 d (J=7.8 Hz, 1H); 7.32-7.35 m (2H); 7.15-7.17 m (2H); 7.06 t (J=7.4 Hz, 1H); 6.97 t (J=7.4 Hz, 1H); 4.88 t (J=5.1 Hz, 1H); 4.23-4.31 m (1H); 3.61 s (3H); 3.45-3.58 m (2H); 3.03, 2.95 AB (J₁=14.3 Hz, J₂=6.6 Hz, 2H); | | | | |
| # 2.38 s (3H); 2.31 s (3H). | 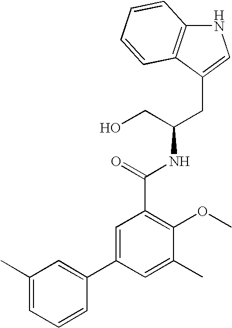 | | | |
| 325 | 4′-Fluoro-4- methoxy-5,3′- dimethylbiphenyl-3- carboxylic acid [(R)-2-hydroxy-1- (1H-indol-3- ylmethyl)ethyl]amide; (D)-Tryptophanol and 4′-Fluoro-4-methoxy-5,3′- dimethylbiphenyl- 3-carboxylic acid | 39 | (DMSO-d₆): 10.81 s (1H); 8.24 d (J=7.8 Hz, 1H); | |
| # 7.69 d (J=7.8 Hz, 1H); 7.59-7.60 m (2H); 7.54 d (J=7.4 Hz, 1H); 7.42-7.45 m (1H); 7.33 d (J=8.2 Hz, 1H); 7.18-7.22 m (2H); 7.06 t (J=7.4 Hz, 1H); 6.97 t (J=7.4 Hz, 1H); 4.89 t (J=5.1 Hz, 1H); 4.23-4.31 m (1H); 3.61 s (3H); 3.45-3.58 m (2H); 3.03, 2.95 AB (J₁=14.5 Hz, | | | | |
| # J₂=7.1 Hz, 2H); 2.30 s (6H). | 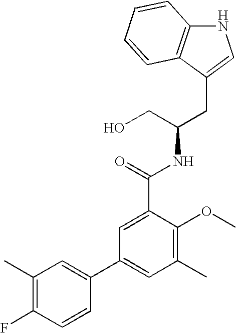 | | | |
| 326 | 3′,4′-Difluoro-4,5′- dimethoxy-5- methylbiphenyl-3- carboxylic acid [(R)-2-hydroxy-1- (1H-indol-3- ylmethyl)ethyl]amide; (D)-Tryptophanol and 3′,4′-Difluoro-4,5′- dimethoxy-5- methylbiphenyl-3- carboxylic acid | 39 | (DMSO-d₆): 10.80 s (1H); | |
| # 8.23 d (J=8.2 Hz, 1H); 7.69 d (J=7.8 Hz, 1H); 7.66, 7.63 AB (J₁=14.8 Hz, J₂1.9 Hz, 2H); 7.32 d (J=8.2 Hz, 1H); 7.18-7.26 m (3H); 7.05 t (J=7.4 Hz, 1H); 6.96 t (J=7.4 Hz, 1H); 4.88 t (J=5.1 Hz, 1H); 4.22-4.30 m (1H); 3.97 s (3H); 3.62 s (3H); 3.45-3.58 | | | | |
| # m (2H); 3.03, 2.95 AB (J₁=14.5 Hz, J₂=6.8 Hz, 2H); 2.31 s (3H). | 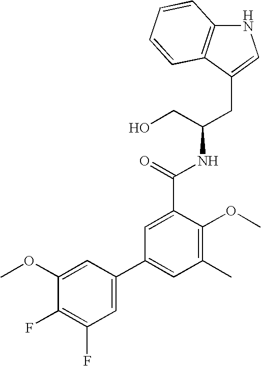 | | | |
| 327 | 3′-Hydroxy-4- isopropoxybi- phenyl-3-carboxylic acid [(R)-1-hydroxy- methyl-2-(1H- indol-3-yl)ethyl]amide; (D)-Tryptophanol and 3′-Hydroxy-4- isopropoxybiphenyl- 3-carboxylic acid | 39 | (DMSO-d₆): 10.82 s (1H); 9.53 s (1H); 8.50 d (J=7.8 Hz, 1H); 8.17 s (1H); 7.71 | |
| # m (2H); 7.34d (J=7.8 Hz, 1H); 7.24 m (2H); 7.16 s (1H); 7.10 m (4H); 6.75 d (J=7.8 Hz, 1H); 4.98 m (1H); 4.79 m (1H); 4.27 m (1H); 3.51 m (2H); 3.00 m (2H); 1.27 m (6H). | 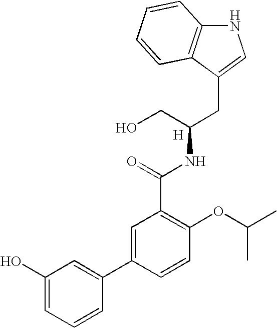 | | | |
| 328 | 3′,4′,5′-Trimethoxy-4- (3-methyl-but-2- enyloxy)biphenyl-3- carboxylic acid [(R)-1-hydroxy- methyl-2-(1H-indol-3- yl)ethyl]amide; (D)-Tryptophanol and 3′,4′,5′-Trimethoxy- 4-(3-methyl-but-2- enyloxy)biphenyl-3- carboxylic acid | 39 | (DMSO-d₆): 10.78 s (1H); 8.31 d (J=8.1 Hz, 1H); | |
| # 8.09 d (J=2.6 Hz, 1H); 7.73 dd (J=2.5 Hz/8.7 Hz, 1H); 7.63 d (J=7.9 Hz, 1H); 7.59 m (2H); 7.31 d (J=7.9 Hz, 1H); 7.21 d (J=8.9 Hz, 1H); 7.10 s (1H); 7.02 m (1H); 6.93 m (1H); 6.81 s (2H); 5.35 m (1H); 4.87 m (1H); 4.62 m (2H); 4.20 m (1H); 3.82 s (6H); 3.65 s (3H); 3.42 m (2H); 2.94 m (2H); 1.66 m (6H). | 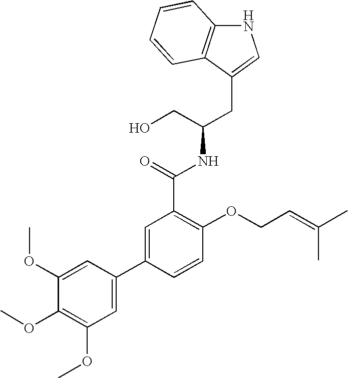 | | | |

### 3′-Butoxy-4-ethoxybiphenyl-3-carboxylic acid [(R)-1-hydroxymethyl-2-(1H-indol-3-yl)ethyl]amide;

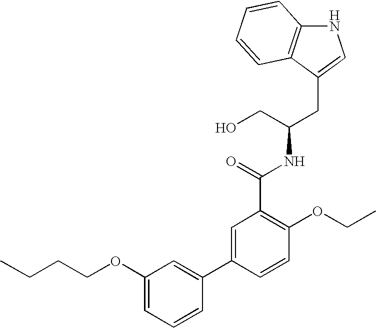

### 329a) 3-n-Butoxyphenylboronic acid pinacol ester

3-Hydroxyphenylboronic acid pinacol ester (1 g), potassium carbonate (1.57 g) and n-butyl iodide (2.6 ml) were dissolved in DMF (20 ml) and stirred at a bath temperature of 100° C. overnight. The cooled reaction mixture was filtered and the filtrate was freed of solvent. The remaining residue was triturated with diisopropyl ether and the residue was filtered off in vacuo and discarded. The mother liquor was concentrated and the crude product was purified by flash chromatography. 710 mg of the title compound were obtained. MS (ESI,+): 277 (M+1).

### 329b) 3′-Butoxy-4-ethoxybiphenyl-3-carboxylic acid [(R)-1-hydroxymethyl-2-(1H-indol-3-yl)ethyl]amide

3-n-Butoxyphenylboronic acid pinacol ester (200 mg), 5-bromo-2-ethoxy-N-[(R)-1-hydroxymethyl-2-(1H-indol-3-yl)ethyl]benzamide (201 mg), dihydrogen dichlorobis(di-tert-butylphosphinito-kappa)dipalladate (12 mg) and potassium carbonate (200 mg) in DMF (5 ml) were stirred at 100° C. overnight. The mixture was diluted with water and extracted several times with ethyl acetate. The combined organic phases were dried over magnesium sulphate and freed of solvent. Purification by HPLC resulted in the title compound in 32% yield (114 mg).

(DMSO-d₆): 10.83 s (1H); 8.41 d (J=8.1 Hz, 1H); 8.16 (J=2.6 Hz, 1H); 7.79 dd (J=2.5 Hz/8.5 Hz, 1H); 7.72 d (J=7.7 Hz, 1H); 7.36 m (2H); 7.20 m (3H); 7.14 m (1H); 7.07 m (1H); 6.98 m (1H); 6.92 dd (J=1.9 Hz/7.9 Hz, 1H); 4.20 m (1H); 4.15 m (2H); 4.05 m (2H); 3.50 m (2H); 3.00 m (2H); 1.73 m (2H); 1.45 m (2H); 1.33 m (3H); 9.96 m (3H).

The following compounds were obtained in analogy to the preparation methods described in detail:

| | Product; | Method | ¹H-NMR (400 MHz) δ | |
|---|---|---|---|---|
| Ex. | reagents | analogous to | [ppm] | Structure |
| 330 | 4-Ethoxy-3′-isopropoxybiphenyl- 3-carboxylic acid [(R)-1- hydroxymethyl-2-(1H-indol-3- yl)ethyl]amide; 5-Bromo-2-ethoxy-N-[(R)-1- hydroxymethyl-2-(1H-indol-3- yl)ethyl]benzamide and 3-Isopropyloxyphenylboronic acid pinacol ester | 329 | (DMSO-d₆): 10.83 s (1H); 8.43 d (J=7.9 Hz, | |
| # 1H); 8.15 d (J=2.6 Hz, 1H); 7.75 dd (J=2.5 Hz/8.5 Hz, 1H); 7.71 d (J=7.7 Hz, 1H); 7.36 m (2H); 7.18 m (2H); 7.11 m (1H); 6.98 m (1H); 6.92 m (1H); 4.72 m (1H); 4.20 m (1H); 4.16 m (2H); 3.51 m (2H); 3.00 m (2H); 1.31 m (9H). | 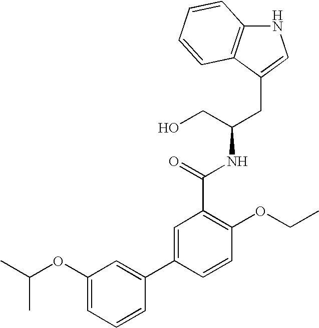 | | | |

### EXAMPLE 331

### N-[(R)-1-Hydroxymethyl-2-(1H-indol-3-yl)ethyl]-5-(7-methoxybenzofuran-2-yl)-2-propoxybenzamide

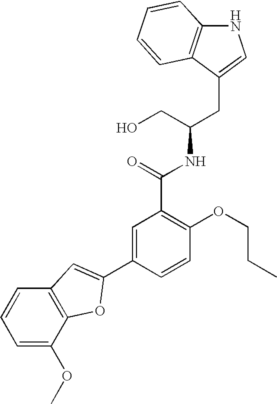

### 331a) Methyl 5-(7-methoxybenzofuran-2-yl)-2-propoxybenzoate

A solution of 7-methoxybenzofuran (500 mg) in THF (3 ml) was cooled to 0° C., and a solution of n-BuLi in hexane (1.6 M, 2.11 ml) was slowly added, whereupon the temperature rose to 15° C. The mixture was then stirred at 5° C. for 1 hour, zinc chloride (1 M solution in THF, 3.71 ml), Pd(PPh₃)₄ (39 mg) and a solution of methyl 5-bromo-2-propoxybenzoate (1.08 g) in THF (3 ml) were added, and the mixture was then stirred under reflux overnight. The mixture was diluted with ethyl acetate and extracted with aqueous ammonium chloride solution. The combined organic phases were dried over sodium sulphate, and the solvent was distilled off in a rotary evaporator. The title compound was obtained after purification by flash chromatography in 11% yield (127 mg). MS (ESI,+): 341 (M+1).

### 331b) 5-(7-Methoxybenzofuran-2-yl)-2-propoxybenzoic acid

A solution of methyl 5-(7-methoxybenzofuran-2-yl)-2-propoxybenzoate (120 mg) in methanol (5 ml) was mixed with potassium hydroxide solution (10% strength in methanol, 2 ml) and stirred at 50° C. for 5 hours. The mixture was concentrated and extracted with MTBE. The aqueous phase was acidified with 1 N HCl and again extracted with MTBE, and the combined organic phases were freed of solvent. The title compound was employed without further purification in the next stage (yield 97%, 112 mg). MS (ESI,+): 327 (M+1).

### 331c) N-[(R)-1-Hydroxymethyl-2-(1H-indol-3-yl)ethyl]-5-(7-methoxybenzofuran-2-yl)-2-propoxybenzamide

5-(7-Methoxybenzofuran-2-yl)-2-propoxybenzoic acid (85 mg) were reacted with (D)-tryptophanol (59 mg) in analogy to general method 113b. The title compound was obtained in 32% yield (41 mg).

(DMSO-d₆): 10.79 s (1H); 8.37 d (J=2.5 Hz, 1H); 8.32 d (J=8.3 Hz, 1H); 7.96 dd (J=2.5 Hz/8.6 Hz, 1H); 7.68 d (J=7.8 Hz, 1H); 7.30 m (2H); 7.24 d (J=8.8 Hz, 1H); 7.15 m (3H); 7.03 m (1H); 6.95 m (1H); 6.90 m (1H); 4.91 m (1H); 4.26 m (1H); 4.06 m (2H); 3.95 s (3H); 3.45 m (2H); 2.96 m (2H); 1.66 m (2H); 0.91 m (3H).

### EXAMPLE 332

### 6-Methoxy-2-(3,4,5-trimethoxyphenyl)quinoline-4-carboxylic acid [(R)-2-(6-chloro-1H-indol-3-yl)-1-hydroxymethylethyl]amide

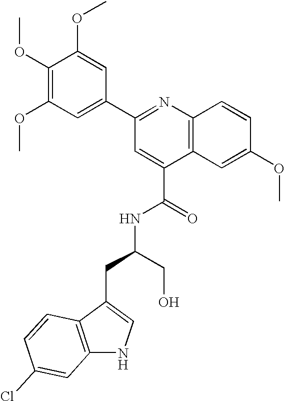

Preswell 0.2 mmol of unloaded Wang resin in 1.5 ml of DMF for 15 min. Then 6 eq of Fmoc-amino acid (R)-3-(6-chloro-1H-indol-3-yl)-2-(9H-fluoren-9-ylmethoxycarbonylamino)propionic acid (0.3M in NMP); 10 eq of pyridine (dried) and 6 eq of 2,4-dichlorobenzoyl chloride (dried) are added and coupled to the resin by shaking for 20 h. After washing 5× with 2 ml of DMF, capping is carried out with 1.5 ml of acetic ahydride 10% in DMF for 5 minutes, followed by washing 5× with 2 ml of DMF. Deprotection with 2 ml of 20% PIP in DMF (1×5 minutes, 1×15 minutes) is followed by washing a further 5× with 2 ml of NMP.

6-Methoxy-2-(3,4,5-trimethoxyphenyl)quinoline-4-carboxylic acid is coupled on by adding 2 eq of 0.3M acid, 6 eq of N-methylmorpholine 3M in NMP+ 2.5% DMAP and 3 eq of HATU 0.3M in NMP (double coupling 2×4 h). This is followed by washing 3× with 2 ml of NMP and 5× with 2 ml of THF. For the reductive elimination, 2 ml of DIBAL 1 M in THF are added at 0° C. under N₂ and stirred for 12 h. Warming to room temperature is followed by filtration and washing with 4×1.5 ml of THF.

HPLC-MS: Column Purospher Star RP C18 4.6×125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5°)

Molecular peak (ESI, M+1): 577

Retention time: 7.96 min.

The following compounds were obtained in analogy to the preparation methods described in detail:

| | Product; | Method | HPLC-MS conditions/ | |
|---|---|---|---|---|
| Ex. | reagents | analogous to | ¹H-NMR (400 MHz) δ [ppm] | Structure |
| 333 | 6-Methoxy-2-(3,4,5- trimethoxyphenyl)quinoline-4- carboxylic acid [(R)-1- hydroxymethyl-2-(2-methyl-1H- indol-3-yl)ethyl]amide;(R)-2-(9H-Fluoren-9-ylmethoxycarbonylamino)-3-(2- methyl-1H-indol-3-yl)-propionic acid and 6-Methoxy-2-(3,4,5- trimethoxyphenyl)quinoline-4- carboxylic acid | 318 | Column Purospher Star | |
| # RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95% (10′) to 95% (2′) to 5% (0.5′) to 5% (2.5′) Molecular peak (ESI, M + 1): 557.6 Retention time: 7.61 min. | 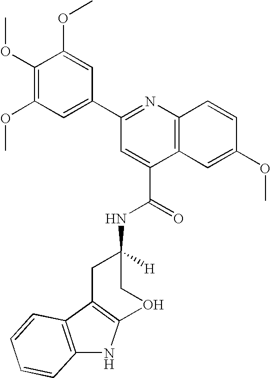 | | | |
| 334 | 6-Methoxy-2-(3,4,5- trimethoxyphenyl)quinoline-4- carboxylic acid [1- hydroxymethyl-2-(6-methyl-1H- indol-3-yl)ethyl]amide; (2-RS)-Amino-3-(6-methyl-1H- indol-3-yl)-propan-1-ol and 6-Methoxy-2-(3,4,5- trimethoxyphenyl)quinoline-4- carboxylic acid | 1 | (DMSO-d₆): 10.65 s (1H); 8.64 d (J=8.6 Hz, 1H); 7.99 d | |
| # (J=9.0 Hz, 1H); 7.96 s (1H); 7.51 d (J=8.5 Hz, 1H); 7.49 s (2H); 7.43 s (1H); 7.40 s (1H); 7.10 d (J=4.3 Hz, 2H); 6.76 d (J=7.8 Hz, 1H); 4.90 t (J=5.4 Hz, 1H); 4.38 m (1H); 3.92 s (6H); 3.73 s (3H); 3.72 s (3H); 3.59 t (J=5.2 Hz, 2H); 2.99 dd (J=14.3 Hz/8.1 Hz, 1H); 2.92 dd (J=14.3 Hz/5.4 Hz, 1H); 2.35 s (3H). | 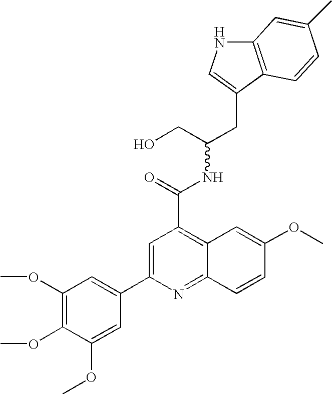 | | | |

### EXAMPLE 335

### N-[(R)-1-(Hydroxymethyl)-2-(1-ethyl)-1H-indol-3-yl)ethyl]-6-methoxy-2-(3-methoxyphenyl)quinoline-4-carboxamide

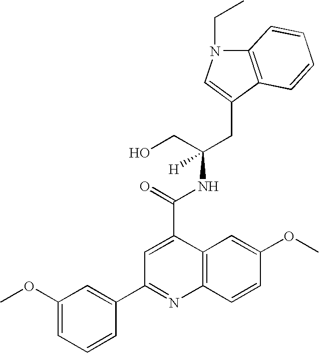

### 335a) Methyl (R)-2-tert-butoxycarbonylamino-3-(1H-indol-3yl)-propionate

15.72 mmol (2.18 ml) of triethylamine were added dropwise to a solution of 3.93 mmol (1 g) of D-tryptophan methyl ester hydrochloride in 35 ml of dichloromethane with stirring and then 7.85 mmol (1.71 g) of di-tert-butyl dicarbonate, dissolved in 5 ml of dichloromethane, were added, followed by 0.39 mmol (48 mg) of dimethylaminopyridine. The mixture was stirred at room temperature for about 1.5 h. Then 25 ml of 10% strength sodium bisulphite solution were added to the reaction mixture and stirred for 15 minutes. After phase separation, the aqueous phase was extracted with dichloromethane. The resulting organic phase was dried over magnesium sulphate, filtered and concentrated in vacuo. Purification by chromatography on silica gel with the eluent cyclohexane/ethyl acetate affords 800 mg of the compound as a white solid.

¹H-NMR (400 MHz, DMSO-d₆):δ [ppm]=10.84 s (1H, NH); 7.45 d (J=7.8 Hz, 1H, aryl) 7.32 d (1H, aryl); 7.14 s (1H, aryl); 7.05 t (J=7.4 Hz, 1H, aryl); 6.97 t (J=7.9 Hz, 1H, aryl); 4.20 m (1H, CH); 3.59 s (3H, OCH₃); 3.08 dd (J=14.4 Hz/5.8 Hz, 1H, CH); 3.00 dd (J=14.4 Hz/8.2 Hz, 1H, CH); 1.33 s (9H, CH₃).

### 335b) Methyl (R)-2-tert-butoxycarbonylamino-3-(1-ethyl-1H-indol-3-yl)propionate

3.27 mmol (183 mg) of potassium hydroxide powder were added in portions to a stirred solution of 2.51 mmol (800 mg) of the protected amino acid prepared in a), in 8 ml of DMSO, slightly cooling in water. This mixture was stirred for 5 minutes and then 3.27 mmol (0.26 ml) of ethyl iodide, dissolved in 2 ml of DMSO, were added dropwise. Stirring was continued at room temperature for 2 hours, and the reaction mixture was then added to saturated aqueous ammonium chloride solution and extracted with ethyl acetate. The resulting organic phase was dried over magnesium sulphate, filtered and concentrated in vacuo with the addition of toluene. 871 mg of the target compound are obtained.

¹H-NMR (400 MHz, DMSO-d₆):δ [ppm]=7.48 d (J=7.8 Hz, 1H, aryl); 7.41 d (J=7.8 Hz, 1H, aryl); 7.23 d (J=7.4 Hz, 1H, aryl); 7.11 t (J=7.6 Hz, 1H, aryl); 7.00 t (J=7.8 Hz, 1H, aryl); 4.20 m (1H, CH); 4.19 q (J=7.0 Hz, 2H, CH₂); 3.07 dd (14.3 Hz/5.3 Hz, 1H, CH); 3.01 dd (J=14.3 Hz/8.2 Hz, 1H, CH); 1.33 s (9H, CH₃); 1.3 t (J=7.0 Hz, 3H, CH₃).

### 335c) Methyl (R)-2-amino-3-(1-ethyl-1H-indol-3-yl)propionate

2.48 mmol (860 mg) of the compound prepared in b) were dissolved in 10 ml of dichloromethane and then 24.8 mmol (1.91 ml) of trifluoroacetic acid were added dropwise at room temperature. After 1 hour, 20 ml of saturated sodium bicarbonate solution were cautiously added dropwise to the mixture until the neutral point was reached. After phase separation, the aqueous phase was extracted with dichloromethane. The resulting organic phase was dried over magnesium sulphate, filtered and concentrated in vacuo. 588 mg of the product are obtained.

¹H-NMR (400 MHz, DMSO-d₆): δ [ppm]=7.47 d (J=7.8 Hz, 1H, aryl); 7.39 d (J=7.8 Hz, 1H, aryl); 7.15 s (1H, aryl); 7.09 t (J=7.5 Hz, 1H, aryl); 6.98 t (J=7.8 Hz, 1H, aryl); 4.14 q (J=7.0 Hz, 2H, CH₂); 3.57 m (1H, CH); 3.54 s (3H, OCH₃); 2.98 dd (J=14.2 Hz/5.4 Hz, 1H, CH); 2.93 dd (J=14.2 Hz/8.4 Hz, 1H, CH); 1.79 s (2H, NH₂); 1.31 t (J=7.0 Hz, 3H, CH₃).

### N-[(R)-1-(Methoxycarbonyl)-2-(1-ethyl)-1H-indol-3-yl)ethyl]-6-methoxy-2-(3-methoxyphenyl)quinoline-4-carboxamide

The title compound was prepared in analogy to general method 1e.

¹H-NMR (400 MHz, DMSO-d₆): δ [ppm]=9.42 d (7.2 Hz, 1H, NH); 8.22 d (J=9.0 Hz, 1H, aryl); 8.05 m (3H, aryl); 7.97 s (1H, aryl); 7.80 d (J=8.6 Hz, 1H, aryl); 7.60 d (J=7.8 Hz, 1H, aryl); 7.45 d (J=8.2 Hz, 1H, aryl); 7.37 t (J=8.6 Hz, 1H, aryl); 7.32 s (1H, aryl); 7.13 t (J=7.8 Hz, 1H, aryl); 7.01 t (J=7.5 Hz, 1H, aryl); 4.80 m (1H, CH); 4.14 q (J=7.0 Hz, 2H, CH₂); 3.96 s (3H, OCH₃); 3.72 s (3H, OCH₃); 3.30 dd (J=14.2 Hz/5.1 Hz, 1H, CH); 3.24 dd (J=14.2 Hz/8.2 Hz, 1H, CH); 1.29 t (J=7.0 Hz, 3H, CH₃).

### 335d) N-[(R)-1-(Hydroxymethyl)-2-(1-ethyl)-1H-indol-3-yl)ethyl]-6-methoxy-2-(3-methoxyphenyl)quinoline-4-carboxamide

0.19 mmol (94 μl) of 2M lithium borohydride solution was added dropwise to a solution of 0.19 mmol (115 mg) of the carboxamide (prepared in analogy to 1e) in 3 ml of THF at 0° C. This mixture is then stirred at room temperature for 4-6 hours. It was then neutralized at 0° C. with 1 N hydrochloric acid and, after addition of water, extracted with ethyl acetate. The organic phase was dried over magnesium sulphate, filtered and concentrated in vacuo. Purification by chromatography on silica gel with the eluent cyclohexane/acetone affords 36.9 mg of pale yellow foam.

¹H-NMR (400 MHz, DMSO-d₆): δ [ppm]=8.76 d (J=7.7 Hz, 1H), 8.20 d (J=9.0 Hz, 1H, aryl) 8.13 m (2H, aryl); 8.06 s (1H, aryl); 8.01 s (1H, aryl); 7.76 d (J=7.4 Hz, 1H, aryl); 7.65 d (J=7.8 Hz, 1H, aryl); 7.40 t (J=7.4 Hz, 2H, aryl); 7.24 s (1H, aryl); 7.10 t (J=7.8 Hz, 1H, aryl); 6.97 t (J=7.4 Hz, 1H, aryl); 4.92 t (J=5.4 Hz, 1H, OH); 4.36 m (1H, CH); 4.14 q (J=7.0 Hz, 2H, CH₂); 3.96 s (3H, OCH₃); 3.60 m (2H, OCH₂); 3.05 dd (J=14.3 Hz/5.6 Hz, 1H, CH); 2.97 dd (J=14.3 Hz/8.2 Hz, 1H, CH); 1.29 t (J=7.0 Hz, 3H, CH₃).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 336 | N-[(R)-1-(Hydroxymethyl)-2-(1- propyl-1H-indol-3-yl) ethyl]-6- methoxy-2-(3,4,5- trimethoxyphenyl)quinoline-4- carboxamide; N-[(R)-1-(Methoxycarbonyl)-2- (1-propyl-1H-indol-3-yl) ethyl]- 6-methoxy-2-(3,4,5- trimethoxyphenyl)quinoline-4- carboxamide | 335 | (DMSO-d₆): 8.66 d (J=8.5 Hz, 1H, NH); 8.00 d (J=8.6 Hz, 1H, | |
| # aryl); 7.98 s (1H, aryl); 7.66 d (J=8.4 Hz, 1H, aryl); 7.50s (2s, aryl); 7.43 m (3H, aryl); 7.22 s (1H, aryl); 7.09 t (J=7.5 Hz, 1H, aryl); 6.97 t (J=7.4 Hz, 1H, aryl); 4.91 t (J=5.3 Hz, 1H, OH); 4.38 m (1H, CH) 4.05 t (J=7.0 Hz, 2H, | | | | |
| # CH₂); 3.92 s (6H, OCH₃); 3.75 (6H, OCH₃); 3.60 m (2H, OCH₂); 3.03 dd (J=14.3 Hz/5.7 Hz, 1H, CH); 2.97 dd (J=14.3 Hz/8.2 Hz, 1H, CH); 1.68 m (2H, CH₂); 0.75 t (J=7.0 Hz, 3H, CH₃). | 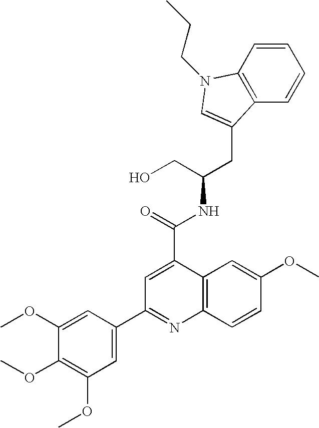 | | | |
| 337 | N-[(R)-1-(Hydroxymethyl)-2-(1- ethyl)-1H-indol-3-yl) ethyl]-2- (3,5-difluoro-4-methoxyphenyl)- 6-methoxyquinoline-4- carboxamide; N-[(R)-1-(Methoxycarbonyl)-2- (1-ethyl)-1H-indol-3-yl) ethyl]- 2-(3,5-difluoro-4-methoxy- phenyl)-6-methoxyquinoline-4- carboxamide | 335 | (DMSO-d₆): 8.63 d (J=8.6 Hz, | |
| # 1H, NH); 7.99 m (3H, aryl), 7.95 s (1H, aryl); 7.67 d (J=7.8 Hz, 1H, aryl), 7.43 m (3H, aryl); 7.25 s (1H, aryl), 7.11 t (J=7.4 Hz, 1H, aryl); 6.98 t (J=7.4 Hz, 1H, aryl); 4.91 t (J=5.5 Hz, 1H, OH); 4.37 m (1H, CH); 4.14 g | | | | |
| # (J=7.0 (1H, CH); 4.14 q (J=7.0 Hz, 2H, CH₂); 4.03 s (3H, OCH₃); 3.60 m (2H, OCH₂); 3.03 dd (J=14.2 Hz/5.3 Hz, 1H, CH); 2.96 dd (J=14.2 Hz/8.3 Hz, 1H, CH); 1.29 t (J=7.0 Hz, 3H, CH₃). | 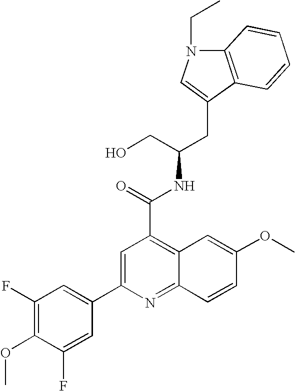 | | | |
| 338 | N-[(R)-1-(Hydroxymethyl)-2-(1- isopropyl-1H-indol-3-yl) ethyl]- 6-methoxy-2 (3-methoxy- phenyl)-quinoline-4- carboxamide; N-[(R)-1- (Methoxycarbonyl)-2-(1- isopropyl-1H-indol-3-yl) ethyl]-6-methoxy-2 (3-methoxy- phenyl)-quinoline-4- carboxamide | 335 | (DMSO-d₆): 8.66 d (J=8.3 Hz, | |
| # 1H, NH); 8.01 d (J=7.7 Hz, 1H, aryl); 7.90 s (1H, aryl); 7.77 s (1H, aryl); 7.74 d (J=7.9 Hz, 1H, aryl): 7.66 d (J=7.8 Hz, 1H, aryl); 7.46 m (4H, aryl), 7.33 s (1H, aryl); 7.08 m (2H, aryl); 6.99 t (J=7.0 Hz, 1H, aryl); 4.92 t (J=5.5 Hz, 1H, OH); | | | | |
| # 4.68 m (1H, CH), 4.38 m (1H, CH), 3.87 s (3H, OCH₃); 3.75 s (3H, OCH₃); 3.60 m (2H, OCH₂); 3.04 dd (J=14.3 Hz/5.5 Hz, 1H, CH); 2.96 dd (J=14.3 Hz/8.3 Hz, 1H, CH); 1.37 (J=7.0 Hz, 6H, CH₃). | 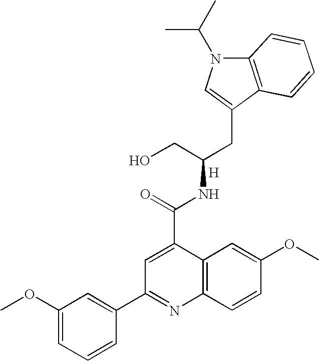 | | | |
| 339 | N-[(R)-1-(Hydroxymethyl)-2-(1- isopropyl-1H-indol-3-yl) ethyl]- 2-(3,5-difluoro-4-methoxy- phenyl)-6-methoxyquinoline-4- carboxamide; N-[(R)-1-(Methoxycarbonyl)-2- (1-isopropyl-1H-indol-3-yl) ethyl]-2-(3,5-difluoro-4- methoxyphenyl)-6- methoxyquinoline-4- carboxamide | 335 | (DMSO-d₆): 8.64 dd (J=8.5 Hz, | |
| # 1H, NH); 8.01 d (J=7.7 Hz, 1H, aryl), 8.00 s (1H, aryl); 7.96 d (J=6.0 Hz, 2H, aryl); 7.66 d (J=7.8 Hz, 1H, aryl); 7.49 s (1H, aryl); 7.45 m (2H, aryl); 7.34 s (1H, aryl) 7.10 t (J=7.4 Hz, 1H, aryl); 7.00 t (J=7.9 Hz, 1H, aryl); 4.92 t (J=5.4 Hz, 1H, OH); 4.69 m (1H, CH); 4.39 m (1H, | | | | |
| # CH); 4.03 s (3H, OCH₃); 3.74 s (3H, OCH₃); 3.60 m (2H, OCH₂); 3.04 dd (J=14.4 Hz/5.6 Hz, 1H, CH); 2.96 dd (J=14.4 Hz/8.4 Hz, 1H, CH); 1.37 t (J=7.0 Hz, 6H, CH₃). | 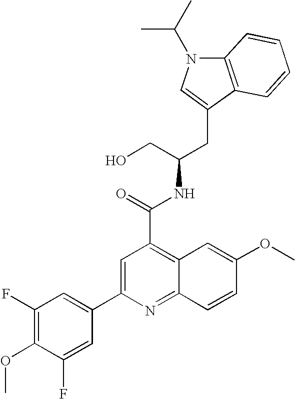 | | | |
| 340 | N-[(R)-1-(Hydroxymethyl)-2-(1- isopropyl-1H-indol-3-yl) ethyl]- 6-methoxy-2-(3,4,5- trimethoxyphenyl)quinoline-4- carboxamide; N[(R)-1-(Methoxycarbonyl)-2- (1-isopropyl-1H-indol-3-yl) ethyl]-6-methoxy-2-(3,4,5- trimethoxyphenyl)quinoline-4- carboxamide | 335 | (DMSO-d₆): 8.66 d (J=8.7 Hz, | |
| # 1H, NH); 8.01 d (J=7.6 Hz, 1H, aryl); 7.99 s (1H, aryl); 7.66 d (J=8.2 Hz, 1H, aryl); 7.50 s (2H, aryl); 7.46 m (3H, aryl); 7.33 s (1H, aryl); 7.10 t (J=7.7 Hz, 1H, aryl); 6.98 t (J=7.6 Hz); 1H, aryl); 4.91 t (J=5.4 Hz, 1H, OH); 4.68 m (1H, CH); 4.41 m | | | | |
| # (1H, CH); 3.91s (6H, OCH₃); 3.75 s (3H, OCH₃); 3.73 s (3H, OCH₃); 3.61 m (2H, OCH₂); 3.03 dd (J=14.4 Hz/5.7 Hz, 1H, CH); 2.97 dd (J=14.4 Hz/8.2 Hz, 1H, CH); 1.36 t (J=6.3 Hz, 6H, CH₃). | 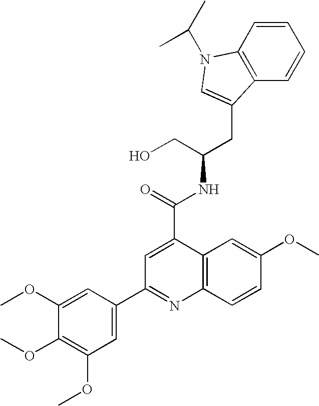 | | | |
| 341 | N-[(R)-1-(Hydroxymethyl)-2-(1- ethyl)-1H-indol-3-yl) ethyl]-2- (3-fluoro-4-methoxyphenyl)-6- trifluoromethoxyquinoline-4- carboxamide N-[(R)-1-(Methoxycarbonyl)-2- (1-ethyl)-1H-indol-3-yl) ethyl)- 2-(3-fluoro-4-methoxyphenyl)-6- trifluoromethoxyquinoline-4- carboxamide | 335 | (DMSO-d₆): 8.76 d (J=7.7 Hz, | |
| # 1H, NH). 8.20 d (J=9.0 Hz, 1H, aryl); 8.13 m (2H, aryl), 8.06 s (1H, aryl); 8.01 s (1H, aryl), 7.76 d (J=7.4 Hz, 1H, aryl); 7.65 d (J=7.8 Hz, 1H, aryl); 7.40 t (J=7.4 Hz, 2H, aryl); 7.24 s (1H, aryl); 7.10 t (J=7.8 Hz, 1H, aryl); 6.97 t (J=7.4 Hz, | | | | |
| # 1H, aryl); 4.92 t (J=5.4 Hz, 1H, OH); 4.36 m (1H, CH); 4.14 q (J=7.0 Hz, 2H, CH₂); 3.96 s (3H, OCH₃); 3.60 m (2H, OCH₂); 3.05 dd (J=14.3 Hz/5.6 Hz, 1H, CH); 2.97 dd (J=14.3 Hz/8.2 Hz, 1H, CH); 1.29 t (J=7.0 Hz, 3H, CH₃). | 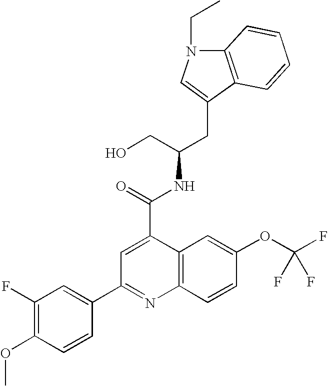 | | | |
| 342 | N-[(R)-1-(Hydroxymethyl)-2-(1- ethyl)-1H-indol-3-yl) ethyl]-2- (7-methoxybenzofuran-2-yl)-6- trifluoromethoxyquinoline-4- carboxamide; N-[(R)-1-(Methoxycarbonyl)-2- (1-ethyl)-1H-indol-3-yl) ethyl]- 2-(7-methoxybenzofuran-2-yl)- 6-trifluoromethoxyquinoline-4- carboxamide | 335 | (DMSO-d₆): 8.86 d (J=8.2 Hz, | |
| # 1H, NH); 8.25 d (J=9.4 Hz, 1H, aryl); 8.11 s 7.83 m (2H, aryl); 7.66 d (J=7.8 Hz, 1H, aryl); 7.42 d (J=8.2 Hz, 1H, aryl); 7.37 d (J=7.8 Hz, 1H, aryl); 7.30 t (J=7.7 Hz, 2H, aryl); 7.25 s (1H, aryl); 7.09 m (2H, aryl); 7.00 t (J=7.1 Hz, 1H, aryl); | | | | |
| # 4.95 t (J=5.4 Hz, 1H, OH); 4.38 m (1H, CH); 4.16 q (J=7.1 Hz, 2H, CH₂); 4.02 s (3H, OCH₃); 3.61 m (2H, OCH₂); 3.05 dd (J=14.2 Hz/5.3 Hz, 1H, CH); 2.94 dd (J=14.2 Hz/8.1 Hz, 1H, CH); 1.29 t (J=7.1 Hz, 3H, CH₃). | 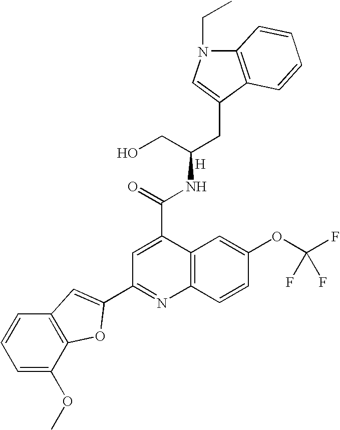 | | | |
| 343 | N-[(R)-1-(Hydroxymethyl)-2-(1- isopropyl-1H-indol-3-yl) ethyl]- 2-(3-fluoro-4-methoxyphenyl)-6- trifluoromethoxyquinoline-4- carboxamide; N-[(R)-1-(Methoxycarbonyl)-2- (1-isopropyl-1H-indol-3-yl) ethyl]-2-(3-fluoro-4- methoxyphenyl)-6- trifluoromethoxyquinoline-4- carboxamide | 335 | (DMSO-d₆): 8.76 d (J=8.6 Hz, | |
| # 1H, NH); 8.20 d (J=9.4 Hz, 1H, aryl); 8.06 m (4H, aryl); 7.77 d (J=9.0 Hz, 1H, aryl), 7.65 d (J=8.2 Hz, 1H, aryl); 7.40 d (J=8.2 Hz, 1H, aryl); 7.37 d (J=9.0 Hz, 1H, aryl); 7.33 s (1H, aryl), 7.09 t (J=7.4 Hz, | | | | |
| # 1H, aryl), 6.97 t (J=7.4 Hz, 1H, aryl); 4.93 t (J=5.5 Hz, 1H, OH); 4.68 m (1H, CH); 4.37 m (1H, CH); 3.96 s (3H, OCH₃); 3.60 m (2H, OCH₂); 3.06 dd (J=14.4 Hz/5.6 Hz, 1H, CH); 2.94 dd (J=14.4 Hz/8.1 Hz, 1H, CH); 1.37 t (J=7.0 Hz, 6H, CH₃). | 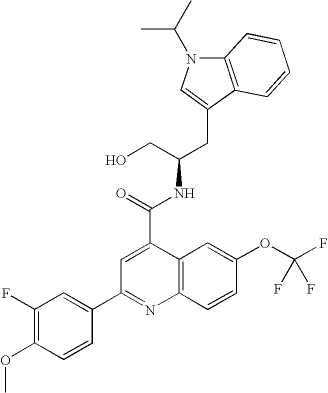 | | | |
| 344 | N-[(R)-1-(Hydroxymethyl)-2-(1- isopropyl-1H-indol-3-yl) ethyl]- 2-(7-methoxybenzofuran-2-yl)- 6-trifluoromethoxyquinoline-4- carboxamide; N-[(R)-1-(Methoxycarbonyl)-2- (1-isopropyl-1H-indol-3-yl) ethyl]-2-(7-methoxybenzofuran- 2-yl)-6-trifluoromethoxy- quinoline-4-carboxamide | 335 | (DMSO-d₆): 8.86 d (J=8.4 | |
| # Hz, 1H, NH); 8.25 d (J=9.4 Hz, 1H, aryl); 8.11 s (1H, aryl); 7.98 s (1H, aryl); 7.82 m (2H, aryl); 7.65 d (J=7.8 Hz, 1H, aryl); 7.44 d (J=8.2 Hz, 1H, aryl); 7.33 m (3H, aryl); 7.29 t (J=8.3 Hz, 1H, aryl); 7.09 m (2H, aryl); 7.00 t (J=7.4 Hz, 1H, aryl); 4.95 t (J=5.5 Hz, 1H, OH); | | | | |
| # 4.70 m (1H, CH); 4.41 m (1H, CH); 4.01 s (3H, OCH₃); 3.61 m (2H, OCH₂); 3.05 dd (J=14.3 Hz/5.5 Hz, 1H, CH); 2.94 dd (J=14.3 Hz/8.2 Hz, 1H, CH); 1.40 d (J=6.3 Hz, 3H, CH₃); 1.37 d (J=6.7 Hz, 3H, CH₃). | 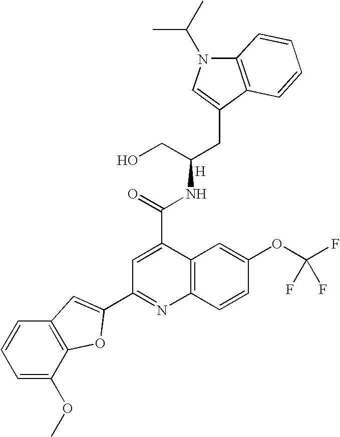 | | | |
| 345 | N-[(R)-1-(Hydroxymethyl)-2-(1- n hexyl-1H-indol-3-yl) ethyl]-6- methoxy-2-(3,4,5- trimethoxyphenyl)quinoline-4- carboxamide; N-[(R)-1-(Methoxycarbonyl)-2- (1-n hexyl-1H-indol-3-yl) ethyl]- 6-methoxy-2-(3,4,5- trimethoxyphenyl)quinoline-4- carboxamide | 335 | (DMSO-d₆): 8.65 d (J=8.6 | |
| # Hz, 1H, NH); 8.00 d (J=7.5 Hz, 1H, aryl); 7.99 S (1H, aryl); 7.65 d (J=8.3 Hz, 1H, aryl); 7.50 s (2H, aryl), 7.45 m (3H, aryl); 7.22 s (1H, aryl); 7.09 t (J=7.6 Hz, 1H, aryl); 6.99 t (J=7.5 Hz, 1H, aryl); 4.91 t (J=5.3 Hz, 1H, OH); 4.39 m (1H, CH); 4.06 t | | | | |
| # (J=7.1 Hz, 2H, CH₂); 3.91 s (6H, OCH₃); 3.75 s (3H, OCH₃); 3.73 s (3H, OCH₃); 3.61 t (J=5.5 Hz, 2H, OCH₂); 3.02 dd (J=14.3 Hz/5.5 Hz, 1H, CH); 2.98 dd (J=14.3 Hz/8.1 Hz, 1H, CH), 1.62 m (2H, CH₂); 1.10 m (6H, CH₂); 0.73 m (3H, CH₃). | 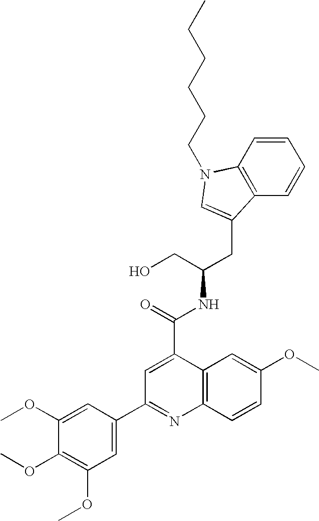 | | | |
| 346 | N-[(R)-1-(Hydroxymethyl)-2-(1- ethyl)-1H-indol-3-yl) ethyl]-4- ethoxy-3′-methoxybiphenyl-3- carboxamide; N-[(R)-1-(Methoxycarbonyl)-2- (1-ethyl)-1H-indol-3-yl) ethyl]- 4-ethoxy-3′-methoxybiphenyl-3- carboxamide | 335 | (DMSO-d₆): 8.42 d (J=7.8 Hz, | |
| # 1H, NH); 8.13 s (1H, aryl); 7.70 d (J=8.5 Hz, 1H, aryl); 7.69 d (J=7.8 Hz, 1H, CH); 7.40 m (2H, aryl); 7.29 m (3H, aryl); 7.12 m (2H, aryl), 6.99 t (J=7.4 Hz, 1H, aryl); 6.92 d (J=6.3 Hz, | | | | |
| # 1H, aryl); 4.94 t (J=5.5 Hz, 1H, OH); 4.24 m (1H, CH); 4.14 q (J=7.0 Hz, 2H, CH₂); 3.82 s (3H, OCH₃); 3.50 m (2H, OCH₂); 2.98 m (2H, CH₂), 1.30 m (6H, CH₃); | 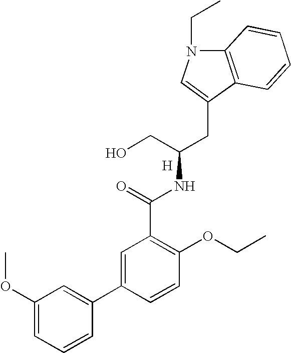 | | | |
| 347 | N-[(R)-1-(Hydroxymethyl)-2-(1- isopropyl)-1H-indol-3-yl) ethyl]- 4-ethoxy-3′-methoxybiphenyl)-3- carboxamide; N-[(R)-1-(Methoxycarbonyl)-2- (1-isopropyl)-1H-indol-3-yl) ethyl]-4-ethoxy-3′- methoxybiphenyl)-3- carboxamide | 335 | (DMSO-d₆): 8.40 d (J=8.2 Hz, | |
| # 1H, NH); 8.13 s (1H, aryl); 7.76 d (J=8.5 Hz, 1H, aryl); 7.70 d (J=7.8 Hz, 1H, aryl); 7.43 d (J=8.2 Hz, 1H, aryl); 7.36 t (J=7.8 Hz, 1H, aryl); 7.29 s (1H, aryl); 7.19 t (J=8.2 Hz, 2H, aryl); 7.13 s (1H, aryl); 7.10 t (J=7.8 Hz, 1H, aryl); 6.98 t (J=7.4 Hz, 1H, aryl); 6.93 d | | | | |
| # (J=7.4 Hz, 1H, aryl); 4.94 s (J=5.4 Hz, 1H, OH); 4.76 m (1H, CH) 4.24 m (1H, CH); 4.14 q (J=7.0 Hz, 2H, CH₂); 3.82 s (3H, OCH₃); 3.47 m (2H, OCH₂); 2.98 m (2H, CH₂); 1.40 t (J=6.6 Hz, 6H, CH₃); 1.31 t (J=7.0 Hz; 3H, CH₃). | 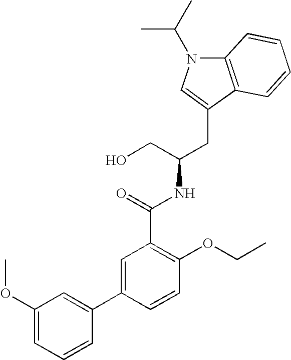 | | | |
| 348 | N-[(R)-2-(1-Ethyl-1H-indol-3- yl)-1-(hydroxymethyl)ethyl]-3′- fluoro-4′-methoxy[1,1′- diphenyl]-3-carboxamide; 1-Ethyl-L-tryptophanol and 3′-Fluoro-4′-methoxy[1,1′- biphenyl]-3-carboxylic acid | 39 | (CDCl₃): 7.73 s (1H); 7.70 d (J= | |
| # 8.0 Hz, 1H); 7.61 d (J=7.6 Hz, 1H); 7.56 d (J=7.8 Hz, 1H); 7.41 dd (J=7.8 Hz/7.6 Hz, 1H); 7.35 d (J=8.0 Hz, 1H); 7.28-7.20 m (3H); 7.12 dd (J=8.0 Hz/7.0 Hz, 1H); 7.04 s (1H); 7.02 dd (J=8.0 Hz/7.0 Hz, 1H); 6.51 d | | | | |
| # (J=6.6 Hz, 1H); 4.48 m (1H); 4.14 q (J=7.3 Hz, 2H); 3.94 s (3H); 3.85 m (1H); 3.80 m (1H); 3.18 dd (J=14.7 Hz/7.1 Hz, 1H); 3.15 dd (J=14.7 Hz/6.6 Hz, 1H); 1.42 t(J=7.3 Hz, 3H). | 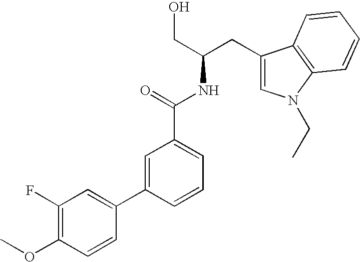 | | | |
| 349 | 3′-Fluoro-N-[(R)-1- (hydroxymethyl)-2-(1-propyl- 1H-indol-3-yl)ethyl]-4′- methoxy[1,1′-biphenyl]-3- carboxamide 1-propyl-L- tryptophanol and 3′-Fluoro-4′- methoxy[1,1′-biphenyl]-3- carboxylic acid | 39 | (DMSO-d₆): 8.28 d (J=8.3 Hz, | |
| # 1H); 8.03 s (1H); 7.78 d (J=7.8 Hz, 1H); 7.76 d (J=7.6 Hz, 1H); 7.68 d (J=8.0 Hz, 1H); 7.65 dd (J=13.1 Hz/2.3 Hz, 1H); 7.53 d (J=8.8 Hz, 1H); 7.49 dd (J=7.8 Hz/7.6 Hz, 1H); 7.28 d (J=8.0 Hz, 1H); 7.28 dd (J=8.8 Hz/8.8 Hz, 1H); 7.17 s (1H); 7.09 dd (J=8.0 Hz/7.0 Hz, 1H); 6.98 dd | | | | |
| # (J=8.0 Hz/7.0 Hz, 1H); 4.82 m (1H); 4.25 m (1H); 4.03 t (J=6.8 Hz, 2H); 3.90 s (3H); 3.55 m (1H); 3.51 m (1H); 3.04 dd (J=14.2 Hz/5.8 Hz, 1H); 2.93 dd (J=14.2 Hz/7.6 Hz, 1H); 1.66 m (2H); 0.71 t (J=7.4 Hz, 3H). | 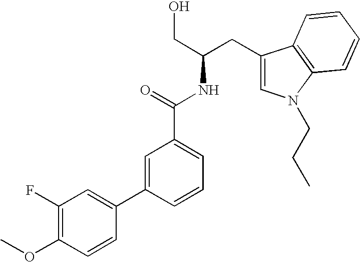 | | | |
| 350 | N-[(R)-2-(1-Butyl-1H-indol-3- yl)-1-(hydroxymethyl)ethyl]-3′- fluoro-4′-methoxyl[1,1′- biphenyl]-3-carboxamide; 1-Butyl-L-tryptophanol and 3′-Fluoro-4′-methoxy[1,1′- biphenyl]-3-carboxylic acid | 39 | (DMSO-d₆): 8.27 d (J=8.3 Hz, | |
| # 1H); 8.04 s (1H); 7.78 d (J=7.8 Hz, 1H); 7.76 d (J=7.8 Hz, 1H); 7.67 d (J=8.0 Hz, 1H); 7.65 dd (J=12.9 Hz/2.3 Hz, 1H); 7.53 d (J=8.8 Hz, 1H); 7.49 dd (J=7.8 Hz/7.8 Hz, 1H); 7.37 d (J=8.0 Hz, 1H); 7.28 dd (J=8.8 Hz/8.8 Hz, 1H); 7.16 s (1H); 7.09 dd (J=8.0 Hz/7.0 Hz, 1H); | | | | |
| # 6.99 dd (J=8.0 Hz/7.0 Hz, 1H); 4.82 m (1H); 4.25 m (1H); 4.06 t (J=6.9 Hz, 2H); 3.90 s (3H); 3.56 m (1H); 3.51 m (1H); 3.04 dd (J=14.4 Hz/5.8 Hz, 1H); 2.93 dd (J=14.4 Hz/7.8 Hz, 1H); 1.61 m (2H); 1.11 m (2H); 0.72 t (J=7.3 Hz, 3H). | 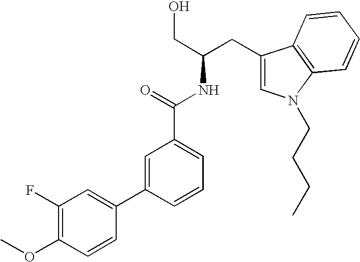 | | | |
| 351 | 3′-Fluoro-N-[(R)-1- (hydroxymethyl)-2-[1-(3- methylbutyl)-1H-indol-3- yl]ethyl]-4′-methoxy[1,1′- biphenyl]-3-carboxamide; 1-(3-Methylbutyl)-L- tryptophanol and 3′-Fluoro-4′-methoxy[1,1′- biphenyl]-3-carboxylic acid biphenyl]-3-carboxylic acid | 39 | (DMSO-d₆): 8.27 d | |
| # (J=8.3 Hz, 1H); 8.04 s (1H); 7.78 d (J=7.8 Hz, 1H); 7.76 d (J=7.6 Hz, 1H); 7.66 d (J=8.0 Hz, 1H); 7.64 dd (J=12.9 Hz/2.3 Hz, 1H); 7.53 d (J=8.8 Hz, 1H); 7.49 dd (J=7.8 Hz/7.6 Hz, 1H); 7.36 d (J=8.0 Hz, 1H); 7.28 dd (J=8.8 Hz/8.8 Hz, 1H); 7.16 s (1H); 7.09 dd (J=8.0 Hz/7.0 Hz, 1H); 6.99 dd | | | | |
| # (J=8.0 Hz/7.0 Hz, 1H); 4.82 m (1H); 4.25 m (1H); 4.07 t (J=6.8 Hz, 2H); 3.89 s (3H); 3.56 m (1H); 3.51 m (1H); 3.04 dd (J=14.4 Hz/5.8 Hz, 1H); 2.93 dd (J=14.4 Hz/8.1 Hz, 1H); 1.51 td (J=7.2 Hz/7.0 Hz, 1H); 1.36 m (2H); 0.77 d (J=7.4 Hz, 6H). | 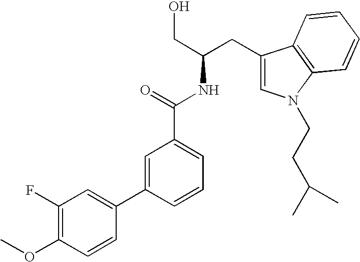 | | | |
| 352 | 3′-Fluoro-N-[(R)-1- (hydroxymethyl)-2-(1-pentyl- 1H-indol-3-yl)ethyl]-4′- methoxy[1,1′-biphenyl]-3- carboxamide; 1-Pentyl-L- tryptophanol and 3′-Fluoro-4′- methoxy[1,1′-biphenyl]-3- carboxylic acid | 39 | (DMSO-d₆): 8.27 d (J=8.1 Hz, | |
| # 1H); 8.03 s (1H); 7.78 d (J=7.8 Hz, 1H); 7.76 d (J=7.6 Hz, 1H); 7.66 d (J=8.0 Hz, 1H); 7.64 dd (J=12.9 Hz/2.3 Hz, 1H); 7.53 d (J=8.8 Hz, 1H); 7.49 dd (J=7.8 Hz/7.6 Hz, 1H); 7.37 d (J=8.0 Hz, 1H); 7.28 dd (J=8.8 Hz/8.8 Hz, 1H); 7.17 s (1H); 7.09 dd | | | | |
| # (J=8.0 Hz/7.0 Hz, 1H); 6.98 dd (J=8.0 Hz/7.0 Hz, 1H); 4.82 m (1H); 4.25 m (1H); 4.05 t (J=6.9 Hz, 2H); 3.89 s (3H); 3.56 m (1H); 3.51 m (1H); 3.04 dd (J=14.7 Hz/5.8 Hz, 1H); 2.93 dd (J=14.7 Hz/8.1 Hz, 1H); 1.63 m (2H); 1.13 m (2H); 1.10 m (2H); 0.70 t (J=7.1 Hz, 3H). | 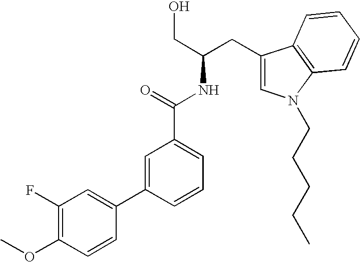 | | | |
| 353 | 3′-Fluoro-N-[(R)-2-(1-hexyl-1H- indol-3-yl)-1- (hydroxymethyl)ethyl]-4′- methoxy[1,1′-biphenyl]-3- carboxamide; 1-Hexyl-L-tryptophanol and 3′-Fluoro-4′-methoxy[1,1′- biphenyl]-3-carboxylic acid | 39 | (DMSO-d₆): 8.27 d (J=8.1 Hz, | |
| # 1H); 8.04 s (1H); 7.78 d (J=7.8 Hz, 1H); 7.76 d (J=7.6 Hz, 1H); 7.66 d (J=8.0 Hz, 1H); 7.65 dd (J=12.9 Hz/2.3 Hz, 1H); 7.53 d (J=8.8 Hz, 1H); 7.49 dd (J=7.8 Hz/7.6 Hz, 1H); 7.37 d (J=8.0 Hz, 1H); 7.28 dd (J=8.8 Hz/8.8 Hz, 1H); 7.17 s (1H); 7.09 dd (J= | | | | |
| # 8.0 Hz/7.0 Hz, 1H); 6.98 dd (J=8.0 Hz/7.0 Hz, 1H); 4.82 m (1H); 4.25 m (1H); 4.05 t (J=6.9 Hz, 2H); 3.89 s (3H); 3.56 m (1H); 3.51 m (1H); 3.04 dd (J=14.4 Hz/5.8 Hz, 1H); 2.93 dd (J=14.4 Hz/8.1 Hz, 1H); 1.61 m (2H); 1.10 m (6H); 0.73 t (J=7.1 Hz, 3H). | 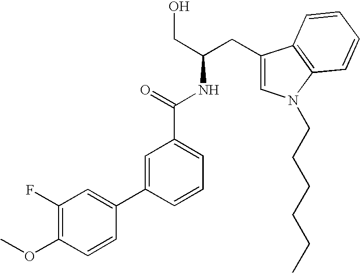 | | | |

### EXAMPLE 354

### 4-Ethoxy-3′-methoxybiphenyl-3-carboxylic acid [2-(5,6-difluoro-1H-indol-3-yl)-1-hydroxymethylethyl]amide

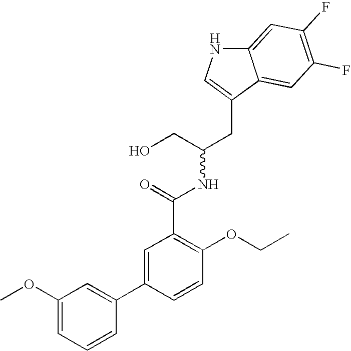

### 354a) (5,6-Difluoro-1H-indol-3-yl)-acetaldehyde

At 0° C., phosphoryl chloride (22.03 g) was slowly added dropwise to DMF (19.1 g), and the mixture was stirred at 0-5° C. for half an hour and then at room temperature for one hour. The mixture was cooled again to 0° C., and a solution of 5,6-difluoro-1H-indole (20 g) in DMF (20 g) was slowly added dropwise. The mixture was stirred at 0° C. for 30 minutes and then at room temperature for a further 15 hours. The reaction mixture was poured onto ice (200 g) and basified to pH 10 with NaOH. The crystalline title compound was filtered off, washed with water and dried in vacuo (yield 22.7 g, 96%). MS (ESI,+): 196 (M+1).

### 354b) [2-(5,6-Difluoro-1H-indol-3-yl)ethyl]diethylamine

Sodium triacetoxyborohydride (26.3 g) was added in portions to a solution of (5,6-difluoro-1H-indol-3-yl)acetaldehyde (15 g) and diethylamine (6.66 g) in absolute dichloromethane (300 ml) with 2 drops of trifluoroacetic acid, and the mixture was stirred at room temperature for 24 hours. The solvent was distilled off in a rotary evaporator, and the residue was mixed with 10% strength aqueous sodium bicarbonate solution and extracted with ethyl acetate. The combined organic phases were dried over sodium sulphate and concentrated in a rotary evaporator. The crude product was purified by flash chromatography, and the title compound was obtained in 68% yield (13.5 g). MS (ESI,+): 253 (M+1).

### 354c) Ethyl 3-(5,6-difluoro-1H-indol-3-yl)-2-nitropropionate

A mixture of gramine (8 g) and ethyl 2-nitroacetate (8.9 g) was stirred in absolute toluene at 90-100° C. for 4 hours. The reaction mixture was concentrated in a rotary evaporator, and the crude product was purified by flash chromatography (chloroform:methanol 19:1), after which the title compound was obtained in a yield of 11.7 g as a 1:2 mixture with ethyl 2-nitroacetate. MS (ESI,+): 299 (M+1).

### 354d) Ethyl 2-amino-3-(5,6-difluoro-1H-indol-3-yl)propionate

The mixture from the above stage was stirred with ammonium formate (9.9 g) and Pd (4.1 g, 10% on activated carbon) in 300 ml of ethanol under reflux for 15 hours. The reaction mixture was concentrated in a rotary evaporator, diluted with water (100 ml) and extracted with ethyl acetate. The combined organic phases were dried over sodium sulphate and concentrated in a rotary evaporator. The residue was purified by flash chromatography (silica, chloroform:methanol 19:1) and recrystallized as HCl salt from ethanol. The title compound was obtained in a yield of 2.7 g. MS (ESI,+): 269 (M+1).

### 354e) 4-Ethoxy-3′-methoxybiphenyl-3-carboxylic acid [2-(5,6-difluoro-1H-indol-3-yl)-1-hydroxymethylethyl]amide

0.39 mmol (143 mg) of the acid was dissolved in 5 ml of dimethylformamide and, at room temperature, 0.39 mmol (59 mg) of 1-hydroxy-1H-benzotriazole hydrate and 0.39 mmol (74 mg) of N-[3-(dimethylamino)propyl]-N′-ethylcarbodiimide hydrochloride were added. The mixture was stirred at the stated temperature for 60 minutes, and then 0.3 mmol (80 mg) of the difluorotryptophan ethyl ester was added. After a further hour, the reaction mixture was added to saturated sodium bicarbonate solution, and the precipitate was filtered and washed with water. Purification by chromatography on silica gel with the eluent cyclohexane/ethyl acetate affords 64 mg of the compound as yellow foam. 0.15 mmol (76 μl) of 2M lithium borohydride solution was added dropwise to a solution of 0.1 mmol (63 mg) of the carboxamide in 2 ml of THF at 0° C. This mixture is then stirred at room temperature for 4-6 hours. It was subsequently neutralized with 1 N hydrochloric acid at 0° C. and, after addition of water, extracted with ethyl acetate. The organic phase was dried over magnesium sulphate, filtered and concentrated in vacuo. Purification by chromatography on silica gel with the eluent cyclohexane/ethyl acetate affords 37 mg of pale yellow powder.

(DMSO-d₆): 11.00 s (1H) 8.40 d (J=7.8 Hz, 1H); 8.12 s (1H); 7.77 d (J=8.6 Hz, 1H); 7.65 m (1H); 7.36 m (3H); 7.20 m (3H); 7.13 s (1H); 6.91 d (J=9.7 Hz, 1H); 4.98 t (J=5.4 Hz, 1H); 4.16 m (3H); 3.82 s (3H); 3.45 m (2H); 2.95 m (2H); 1.33 t (J=7.0 Hz, 3H).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 355 | 6-Methoxy-2-(3,4,5- trimethoxyphenyl)quinoline-4- carboxylic acid [2-(5,6-difluoro- 1H-indol-3-yl)-1- hydroxymethylethyl]amide; 2-Amino-3-(5,6-difluoro-1H- indol-3-yl)-propan-1-ol and 6-Methoxy-2-(3,4,5- trimethoxyphenyl)quinoline-4- carboxylic acid | 354 | (DMSO-6): 11.00 s (1H); 7 d (J=8.6 Hz, 1H); 8.00 d (J=9.0 Hz, 1H); | |
| # 7.96 s (1H); 7.59 m (1H); 7.49 s (2H); 7.42 d (6.6 Hz, 1H); 7.31 m (1H); 7.29 s (1H); 4.93 t (J=5.3 Hz, 1H); 4.35 m (1H); 3.92 s (6H); 3.75 s (6H); 3.60 t (J=5.5 Hz, 2H); 3.00 dd (J=14.3 Hz/5.6 Hz, 1H); 2.90 dd (J=14.4 Hz/8.0 Hz, 1H). | 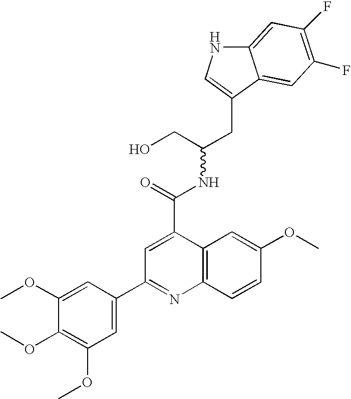 | | | |

### EXAMPLE 356

### N-[(R)-1-(Hydroxymethyl)-2-(1-ethyl-5-fluoro-1H-indol-3-yl)ethyl]-6-methoxy-2-(3,4,5-trimethoxyphenyl)quinoline-4-carboxamide

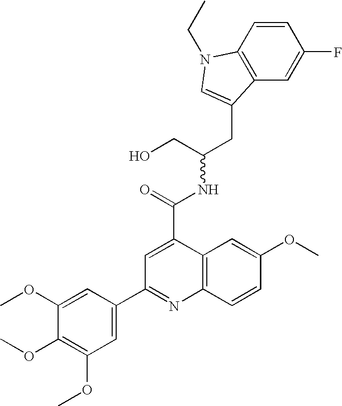

0.2 mmol (12 mg) of potassium hydroxide powder was added in portions, cooling slightly with water, to a stirred solution of 0.09 mmol (50 mg) of 6-methoxy-2-(3,4,5-trimethoxyphenyl)quinoline-4-carboxylic acid [R-1-(5-fluoro-1H-indol-3-ylmethyl)-2-hydroxyethyl]amide in 1 ml of DMSO. This mixture was stirred for 5 minutes and then 0.2 mmol (17.2 μl) of ethyl iodide, dissolved in 0.3 ml of DMSO, was added dropwise. Stirring was then continued at room temperature for 2 hours, and the reaction mixture was subsequently added to saturated aqueous ammonium chloride solution and extracted with ethyl acetate. The resulting organic phase was dried over magnesium sulphate, filtered and concentrated in vacuo with addition of toluene. Purification by chromatography on silica gel with the eluent cyclohexane/acetone affords 41.9 mg of the compound as pale yellow foam.

(DMSO-d₆): 8.67 d (J=8.6 Hz, 1H, NH); 8.01 d (J=8.5 Hz, 1H, aryl); 7.98 s (1H, aryl); 7.50 s (2H, aryl); 7.42 m (4H, aryl); 7.32 s (1H, aryl); 6.94 t (J=7.3 Hz, 1H, aryl), 6.94 t (J=7.3 Hz, 1H, aryl); 4.91 t (J=5.4 Hz, 1H, OH); 4.36 m (1H, CH); 4.13 q (J=7.0 Hz, CH₂); 3.92 s (6H, OCH₃); 3.60 t (J=5.5 Hz, 2H, OCH₂); 2.98 dd (J=14.4 Hz/5.7 Hz, 1H, CH); 2.92 dd (J=14.4 Hz/8.1 Hz, 1H, CH); 1.27 t (J=7.0 Hz, CH₃).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 357 | 6-Methoxy-2-(3,4,5- trimethoxyphenyl)quinoline-4- carboxylic acid [(R)-2-(1-ethyl-5- fluoro-1H-indol-3-yl)-1- hydroxymethylethyl]amide; 6-Methoxy-2-(3,4,5- trimethoxyphenyl)quinoline-4- carboxylic acid[(R)-1-(5-fluoro- 1H-indol-3-ylmethyl)-2- hydroxyethyl]amide | 356 | (DMSO-d₆): 8.67 d (J=8.6 Hz, | |
| # 1H, NH); 8.01 d (J=8.5 Hz, 1H, aryl); 7.98 s (1H, aryl); 7.50 s (2H, aryl); 7.42 m (4H, aryl); 7.32 s (1H, aryl); 6.94 t (J=7.3 Hz, 1H, aryl), 6.94 t (J=7.3 Hz, 1H, aryl); 4.91 t (J=5.4 Hz, 1H, OH); 4.36 m (1H, CH); 4.13 q | | | | |
| # (J=7.0 Hz, CH₂); 3.92 s (6H, OCH₃); 3.60 t (J=5.5 Hz, 2H, OCH₂); 2.98 dd (J=14.4 Hz/5.7 Hz, 1H, CH); 2.92 dd (J=14.4 Hz/8.1 Hz, 1H, CH); 1.27 t (J=7.0 Hz, CH₃). | 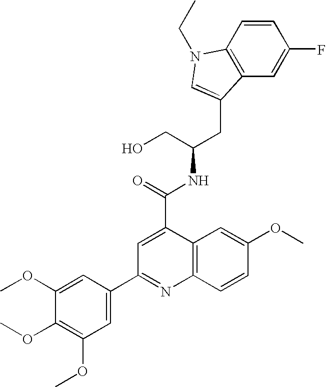 | | | |

### EXAMPLE 358

### 6-(3,4,5-Trimethoxyphenyl)quinoline-8-carboxylic acid [(R)-1-hydroxymethyl-2-(1H-indol-3-yl)ethyl]amide

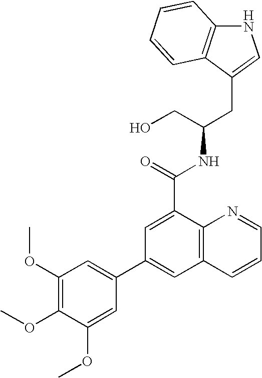

### 358a) 6-Bromoquinoline-8-carboxylic acid

Concentrated sulphuric acid (20.5 ml) was added to a solution of 2-amino-5-bromobenzoic acid (25 g), glycerol (35 ml) and nitrobenzene (7.3 ml) (highly exothermic) and the reaction was stirred at 150° C. for 5 hours. The cooled reaction mixture was poured into ice-water (750 ml), and KOH (22.4 g) was added. The precipitate was filtered off, and the residue on the filter was dissolved in KOH (5 g) in water (350 ml). Activated carbon was added, and the mixture was stirred at 50° C. for half an hour. The mixture was filtered through a short layer of silica gel, and the filtrate was acidified with acetic acid. The resulting precipitate was filtered off, washed with water and dried in air. Recrystallization from acetonitrile yielded 7.5 g (26%) of the title compound. MS (ESI,+): 253 (M+1).

### 358b) 6-Bromoquinoline-8-carboxylic acid [(R)-1-hydroxymethyl-2-(1H-indol-3-yl)ethyl]amide

The quinolinecarboxylic acid was reacted with (D)-tryptophanol to give the title compound in analogy to general method 113b.

### 358c) 6-(3,4,5-Trimethoxyphenyl)quinoline-8-carboxylic acid [(R)-1-hydroxymethyl-2-(1H-indol-3-yl)ethyl]amide

The aryl bromide was arylated under the Suzuki conditions to give the title compound in analogy to general method 125e.

(DMSO-d₆): 11.17 d (J=7.8 Hz, 1H); 10.83 s (1H); 8.87 s (2H); 8.52 dd (J=1.5 Hz/8.3 Hz, 1H); 8.44 s (1H); 7.73 d (J=7.8 Hz, 1H); 7.60 m (2H); 7.33 d (J=7.8 Hz, 1H); 7.26 s (1H); 7.05 m (3H); 6.94 m (1H); 5.03 m (1H); 4.42 m (1H); 3.88 s (6H); 3.71 s (3H); 3.67 m (1H); 3.54 m (1H); 3.12 m (2H).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 359 | 3-(3,4,5-Trimethoxyphenyl)- naphthalene-1-carboxylic acid [(R)-1-hydroxymethyl-2-(1H- indol-3-yl)ethyl]amide; 3-Bromo-naphthalene-1- carboxylic acid [(R)-1- hydroxymethyl-2-(1H-indol-3- yl)ethyl]amide and 3,4,5- Trimethoxyphenylboronic acid | 113b 125e | (DMSO-d₆): 10.79 s (1H); 8.40 d | |
| # (J=8.3 Hz, 1H); 8.24 s (1H); 7.99 m (1H); 7.75 d (J=1.7 Hz, 1H); 7.65 d (J=7.7 Hz, 1H); 7.52 m (1H); 7.44 m (1H); 7.30 d (J=7.9 Hz, 1H); 7.18 s (1H); 7.03 m (4H); 6.92 m (1H); 4.83 m (1H); 4.33 m (1H); 3.87 s (6H); 3.70 s (3H); 3.58 m (1H); 3.51 m (1H); 3.04 m (1H); 2.92 m (1H). | 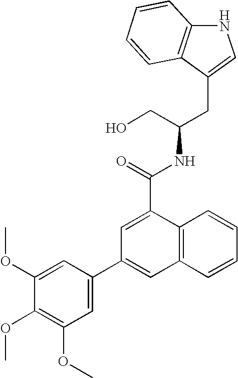 | | | |
| 360 | 4-Methoxy-5-(3,4,5- trimethoxyphenyl)thiophene-3- carboxylic acid [(R)-2-hydroxy- 1-(1H-indol-3-ylmethyl) ethyl]amide; 5-Bromo-4- methoxy-thiophene-3-carboxylic acid [(R)-2-hydroxy-1- (1H-indol-3-ylmethyl)ethyl]amide and 3,4,5-Trimethoxy- phenylboronic acid | 113b 125e | (CDCl₃): 10.81 s (1H); 7.95 s | |
| # (1H); 7.88 d (J=8.3 Hz, 1H); 7.68 d (J=7.8 Hz, 1H); 7.33 (J=7.8 Hz, 1H); 7.17 s (1H); 7.06 m (1H); 6.98 m (1H); 6.90 s (2H); 4.94 m (1H); 4.25 m (1H); 3.82 s (6H); 3.70 s (3H); 3.54 s (3H); 3.51 m (1H); 3.46 m (1H); 2.97 m (2H). | 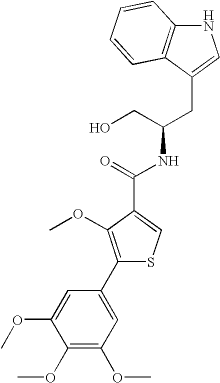 | | | |
| 361 | 6-(3,4,5-Trimethoxyphenyl)-1H- benzoimidazole-4-carboxylic acid [(R)-2-hydroxy-1-(1H- indol-3-ylmethyl)ethyl]amide; 6-Bromo-1H-benzimidazole-4- carboxylic acid [(R)-2-hydroxy- 1-(1H-indol-3-ylmethyl)ethyl]amide and 3,4,5-Tri- methoxyphenylboronic acid | 113b 125e | (DMSO-d₆): 12.94 s | |
| # (1H); 10.73 s (1H); 10.02 d (J=7.7 Hz, 1H); 8.47 s (1H); 8.10 s (1H); 7.93 s (1H); 7.70 d (J=7.7 Hz, 1H); 7.28 d (J=7.9 Hz, 1H); 7.14 s (1H); 6.91 m (4H); 4.92 m (1H); 4.33 m (1H); 3.85 s (6H); 3.68 s (3H); 3.56 m (2H); 2.98 m (2H). | 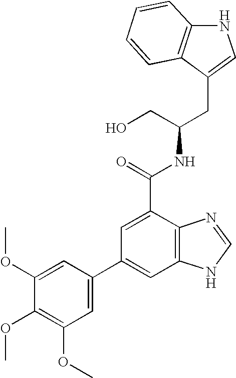 | | | |
| 362 | 2-(3,4,5-Trimethoxyphenyl) thiazole-4-carboxylic acid [(R)-2-hydroxy-1-(1H-indol-3- ylmethyl)ethyl]amide; (D)-Tryptophanol and 2-(3,4,5- Trimethoxyphenyl)thiazole-4- carboxylic acid | 125 | (DMSO-d₆): 10.77 s (1H); | |
| # 8.23 s (1H); 8.04 d (J=8.6 Hz, 1H); 7.66 d (J=7.8 Hz, 1H); 7.28 d (J=8.1 Hz, 1H); 7.23 s (2H); 7.15 s (1H); 7.01 m (1H); 6.92 m (1H); 4.91 m (1H); 4.19 (1H); 3.87 s (6H); 3.70 s (3H); 3.56 m (1H); 3.49 m (1H); 2.99 m (2H). | 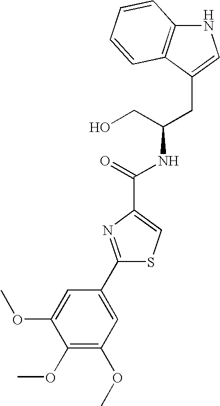 | | | |
| 363 | 5-(3,4,5-Trimethoxyphenyl) thiophene-2-carboxylic acid [(R)-2-hydroxy-1-(1H-indol-3- ylmethyl)ethyl]amide; (D)-Tryptophanol and 5-(3,4,5-Trimethoxyphenyl) thiophene-2-carboxylic acid | 125 | (DMSO-d₆): 10.73 s (1H); | |
| # 8.17 d (J=8.3 Hz, 1H); 7.91 s (1H); 7.74 d (J=4.0 Hz, 1H); 7.60 d (J=7.8 Hz, 1H); 7.48 d (J=3.8 Hz, 1H); 7.28 d (J=8.1 Hz, 1H); 7.10 s (1H); 7.01 m (1H); 6.90 m (1H); 4.80 m (1H); 4.15 m (1H); 3.81 s (6H); 3.64 s (3H); 3.48 m (2H); 2.97 m (1H); 2.85 m (1H). | 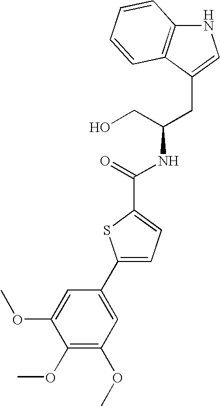 | | | |
| 364 | 5-(3,4,5-Trimethoxyphenyl)- benzo[b]thiophene-2-carboxylic acid [[(R)-2-hydroxy-1-(1H- indol-3-ylmethyl)ethyl]amide; (D)-Tryptophanol and 5-(3,4,5-Trimethoxyphenyl)- benzo[b]thiophene-2-carboxylic acid | 125 | (DMSO-d₆): 10.73 s (1H); | |
| # 8.52 d (J=8.1 Hz, 1H); 8.16 d (J=1.5 Hz, 1H); 8.14 s (1H); 8.02 d (J=8.3 Hz, 1H); 7.73 dd (J=2.0 Hz/8.6 Hz, 1H); 7.61 d (J=8.1 Hz, 1H); 7.28 d (J=8.1 Hz, 1H); 7.12 s (1H); 7.01 m (1H); 6.95 s (2H); 6.92 m (1H); 4.82 m (1H); 4.18 m (1H); 3.85 s (6H); 3.67 s (3H); 3.50 m (2H); 3.01 m (1H); 2.95 m (1H). | 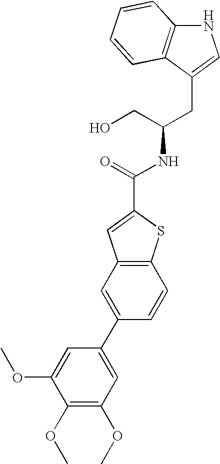 | | | |
| 365 | 2-(3-Fluoro-4-methoxyphenyl) N-[(R)-2-hydroxy-1-(1H-indol- 3-ylmethyl)ethyl]-6-methyl- isonicotinamide; (D)-Tryptophanol and 2-(3-Fluoro-4-methoxyphenyl)- 6-methyl-isonicotinic acid | 125 | (DMSO-d₆): 10.74 s (1H); | |
| # 8.46 d (J=8.3 Hz, 1H); 7.91 m (3H); 7.62 d (J=7.7 Hz, 1H); 7.47 s (1H); 7.26 m (2H); 7.11 s (1H); 7.01 m (1H); 6.93 m (1H); 4.81 m (1H) 2.53 s (3H). | 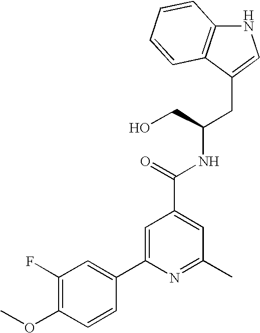 | | | |
| 366 | 2-(3-Fluoro-4-methoxyphenyl)- 6-methylpyrimidine-4-carboxylic acid [(R)-2-hydroxy-1-(1H- indol-3-ylmethyl)ethyl]amide; (D)-Tryptophanol and 2-(3-Fluoro-4-methoxyphenyl)- 6-methylpyrimidine-4-carboxylic acid | 125 | (CDCl₃): 8.39 d (J=Hz, 1H); | |
| # 8.31 s (1H); 8.23 d (J=8.4 Hz, 1H); 8.00 dd (J=12.9 Hz/2.3 Hz, 1H); 7.81 s (1H); 7.72 d (J=7.8 Hz, 1H); 7.42 d (J=8.1 Hz, 1H); 7.22 m (2H); 7.12 m (1H); 7.01 m (1H); 4.49 m (1H); 7.01 m (1H); 4.49 m (1H); 3.97 s (3H); 3.89 m (2H); 3.20 m (2H); 2.71 s (3H). | 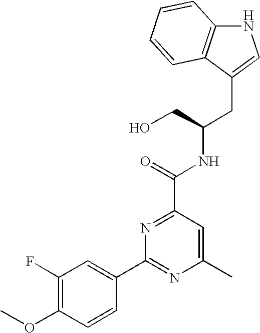 | | | |
| 367 | 6-(4-Methoxyphenyl)pyrimidine- 4-carboxylic acid [(R)-1- hydroxymethyl-2-(1H-indol-3- yl)ethyl]amide; (D)-Tryptophanol and 6-(4-Methoxyphenyl)pyrimidine- 4-carboxylic acid | 125 | (DMSO-d₆): 10.79 s (1H); | |
| # 9.28 s (1H); 8.69 d (J=8.9 Hz, 1H); 8.42 s (1H); 8.27 d (J=9.0 Hz, 1H); 7.70 d (J=7.7 Hz, 1H); 7.33 d (J=7.9 Hz, 1H); 7.14 m (3H); 7.06 m (1H); 6.98 m (1H); 4.28 m (1H); 3.87 s (3H); 3.52 m (2H); 3.03 m (2H). | 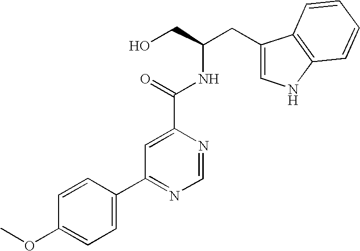 | | | |

### EXAMPLE 368

### 2-(3-Fluoro-4-methoxyphenyl)-6-methoxyquinazoline-4-carboxylic acid [(R)-2-hydroxy-1-(1H-indol-3-ylmethyl)ethyl]amide;

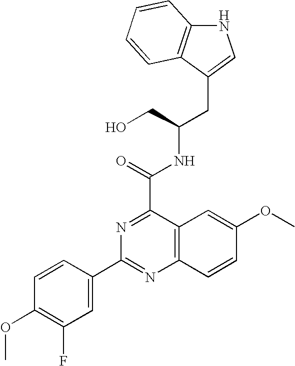

### 368a) 5-Methoxyisatin sodium salt

A solution of 1 N KOH (100 ml) was slowly added in portions to a suspension of 5-methoxyisatin (17.7 g) in water (100 ml), and the mixture was heated to about 40° C. It was stirred until almost all the isatin had dissolved. The undissolved residue was filtered off, and the filtrate was evaporated to dryness in a rotary evaporator. Absolute ethanol (200 ml) was added to the residue, and the solid was stirred at room temperature, and the sodium salt of 5-methoxyisatin was filtered off and dried in vacuo at room temperature. Yield 20.6 g (96%).

### 368b) [2-(3-Fluoro-4-methoxybenzoylamino)-5-methoxyphenyl]oxoacetic acid

Dimethylaminopyridine (3.5 g), and then triethylamine (75 ml) and subsequently a solution of 3-fluoro-4-methoxybenzoyl chloride (37.7 g) in THF (200 ml) were added dropwise to a solution of the sodium salt of 5-methoxyisatin (21.5 g) in THF (300 ml), and the reaction mixture was stirred at room temperature for 20 hours. Water (30 ml) was added to the reaction mixture and stirred for a 4 hours. The insoluble residue was filtered off and the filtrate was evaporated to dryness. The residue was again dissolved in water (900 ml) and acidfied to pH 1 with 1 N HCl. The precipitate which separated out was filtered off, washed with water and dried in air. Recrystallization from benzene yielded 11.1 g (32%) of the title compound. MS (ESI,+): 348 (M+1).

### 368c) 2-(3-Fluoro-4-methoxyphenyl)-6-methoxyquinazoline-4-carboxylic acid

Anhydrous ammonia (5 g) was added to a solution of [2-(3-fluoro-4-methoxybenzoylamino)-5-methoxyphenyl]oxoacetic acid (3.47 g) in ethanol (50 ml). The reaction mixture was heated in a sealed tube at 120° C. under autogenous conditions for 6 hours. Solvent and ammonia were distilled out in a rotary evaporator, and the dry residue was suspended in water (100 ml) and acidified to pH 3-4 with acetic acid. The resulting precipitate was filtered off, washed with water and recrystallized from ethanol in an autoclave at 150° C. Yield 2.1 g (65%). MS (ESI,+): 329 (M+1).

### 368d) 2-(3-Fluoro-4-methoxyphenyl)-6-methoxyquinazoline-4-carboxylic acid [(R)-2-hydroxy-1-(1H-indol-3-ylmethyl)ethyl]amide

The title compound was obtained by reaction with (D)-tryptophanol in analogy to general method 113b.

(DMSO-d₆): 10.81 s (1H); 8.90 d (J=8.6 Hz, 1H); 8.31 m (2H); 8.12 s (1H); 7.97 m (1H); 7.34 m (2H); 7.21 s (1H); 7.00 m (1H); 6.90 m (1H); 4.95 m (1H); 4.35 m (1H); 3.93 s (3H); 3.82 m (3H); 3.58 m (2H); 3.08 m (1H); 3.03 m (1H).

The following compounds were obtained in analogy to the preparation methods described in detail:

| | Product; | Method | ¹H-NMR (400 MHz) δ | |
|---|---|---|---|---|
| Ex. | reagents | analogous to | [ppm] | Structure |
| 369 | 2-(3-Fluoro-4-methoxyphenyl)- 6-iodoquinazoline-4-carboxylic acid [(R)-2-hydroxy-1-(1H- indol-3-ylmethyl)ethyl]amide; (D)-Tryptophanol and 2-(3-Fluoro-4-methoxyphenyl)- 6-iodoquinazoline-4-carboxylic acid | 368 | (DMSO-d₆): 10.81 s | |
| # (1H); 9.11 s (1H); 8.95 d (J=Hz, 1H); 8.35 m (2H); 8.23 m (1H); 7.81 d (J=8.8 Hz, 1H); 7.66 d (J=7.8 Hz, 1H); 7.29 m (2H); 7.21 s (1H); 7.00 m (1H); 6.90 m (1H); 4.93 m (1H); 4.32 m (1H); 3.94 s (3H); 3.58 m (2H); 3.05 m (2H). | 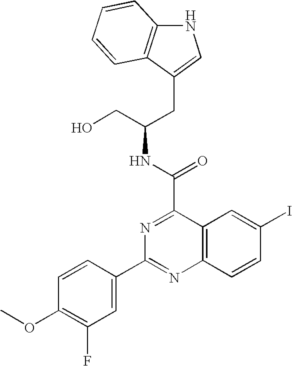 | | | |
| 370 | 2-(4-Methoxyphenyl)- quinazoline-4-carboxylic acid [(R)-1-hydroxymethyl-2-(1H- indol-3-yl)ethyl]amide; (D)-Tryptophanol and 2-(4-methoxyphenyl)- quinazoline-4-carboxylic acid | 368 | (DMSO-d₆): 10.84 s (1H); | |
| # 8.43 d (J=8.6 Hz, 1H); 8.48 m (2H); 8.39 d (J=8.3 Hz, 1H); 7.98 m (2H); 7.66 m (1H); 7.59 m (1H); 7.33 d (J=7.3 Hz, 1H); 7.21 d (J=2.0 Hz, 1H); 7.12 m (2H); 7.03 m (1H); 6.94 m (1H); 4.93 m (1H); 4.36 m (1H); 3.85 s (3H); 3.56 m (2H); 3.08 m (1H); 3.01 m (1H). | 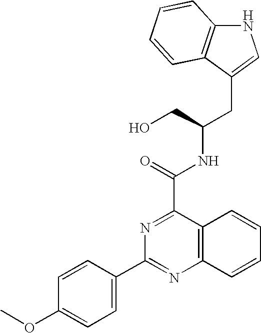 | | | |
| 371 | 2-(3-Fluoro-4-methoxyphenyl)- 6-methoxy-quinazoline-4- carboxylic acid [(R)-1-(1-ethyl- 1H-indol-3-ylmethyl)-2- hydroxyethyl]amide; (R)-2-Amino-3-(1-ethyl-1H- indol-3-yl)-propan-1-ol and 2-(3-Fluoro-4-methoxyphenyl)- 6-methoxy-quinazoline-4- carboxylic acid | 368 | (DMSO-d₆): 8.94 d (J=8.6 Hz, | |
| # 1H); 8.34 m (2H); 8.17 s (1H); 8.00 d (J=9.4 Hz, 1H); 7.69 d (J=7.5 Hz, 2H) 7.38 t (J=10.9 Hz, 2H); 7.28 s (1H); 7.08 t (J=7.5 Hz, 1H; 6.96 t (J=7.1 Hz, 1H); 4.98 t (J=5.4 Hz, 1H); 4.37 m (1H); 4.12 q (J=7.0 Hz, 2H); 3.64 m (2H); 3.10 dd (J=14.3 Hz/5.6 Hz, 1H); 3.03 dd (J=14.3 Hz/8.2 Hz, 1H); 1.26 t (J=7.0 Hz, 3H). | 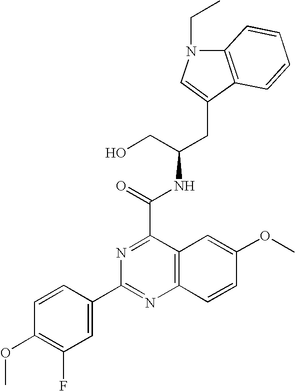 | | | |

### EXAMPLE 372

### 2-(3,4,5-Trimethoxyphenyl)quinoline-4-carboxylic acid [(R)-2-hydroxy-1-(1H-indol-3-ylmethyl)-2-methylpropyl]amide;

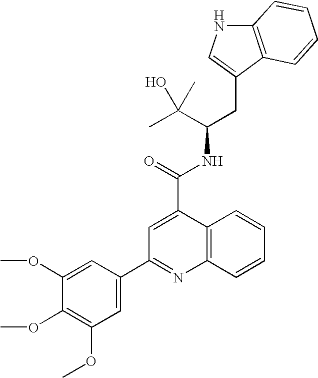

3.33 mmol (1.11 ml) of 3M methylmagnesium bromide solution were added dropwise to a solution of 0.22 mmol (120 mg) of methyl (R)-3-(1H-indol-3-yl)-2-{[2-(3,4,5-trimethoxyphenyl)quinoline-4-carbonyl]amino}propionate in 4.5 ml of THF at 0° C. This mixture is then stirred at room temperature for about 30 minutes and then added to saturated aqueous ammonium chloride solution and extracted with ethyl acetate. The organic phase was dried over magnesium sulphate, filtered and concentrated in vacuo. Purification by chromatography on silica gel with the eluents cyclohexane/ethyl acetate affords 107 mg of pale red foam.

(DMSO-d₆):10.81 s (1H); 8.50 d (J=9.7 Hz, 1H); 8.04 d (J=8.7 Hz, 1H); 7.74 s (1H); 7.72 d (J=7.4 Hz, 1H); 7.62 d (J=7.9 Hz, 1H); 7.49 d (J=8.3 Hz, 1H); 7.46 s (2H); 7.41 d (J=7.8 Hz, 1H); 7.36 d (J=8.2 Hz, 1H); 7.18 s (1H); 7.06 t (J=7.8 Hz, 1H); 6.96 t (J=7.4 Hz, 1H); 4.68 s (1H); 4.40 t (J=9.8 Hz, 1H); 3.92 s (6H); 3.74 s (3H); 3.26 d (J=14.1 Hz, 1H); 2.83 t (J=14.4 Hz, 1H); 1.38 s (3H); 1.27 s (3H).

The following compounds were obtained in analogy to the preparation methods described in detail:

| | Product; | Method | ¹H-NMR (400 MHz) δ | |
|---|---|---|---|---|
| Ex. | reagents | analogous to | [ppm] | Structure |
| 373 | 6-Methoxy-2-(3,4,5- trimethoxyphenyl)quinoline-4- carboxylic acid [(R)-2-hydroxy- 1-(1H-indol-3-ylmethyl)-2- methylpropyl]amide; Methyl (R)-3-(1H-indol-3-yl)-2- {[6-methoxy-2-(3,4,5- trimethoxyphenyl)quinoline-4- carbonyl]amino}propionate | 372 | (DMSO-d₆): 10.77 s (1H); | |
| # 8.53 d (J=9.4 Hz, 1H); 7.97 d (J=9.0 Hz, 1H); 7.68 s (1H); 7.60 d (J=7.8 Hz, 1H); 7.41 s (3H); 7.31 d (J=8.2 Hz, 1H); 7.21 d (J=9.8 Hz, 2H); 7.04 t (J=7.7 Hz, 1H); 6.94 t (J=7.8 Hz, 1H); 4.69 s (1H); 4.38 t (J=10.1 Hz, 1H); 3.92 s (6H); 3.75 s (3H); 3.63 s (3H); 3.24 d (J=14.0 Hz, 1H); 2.82 t (J=14.7 Hz, 1H); 1.37 s (3H); 1.27 s (3H). | 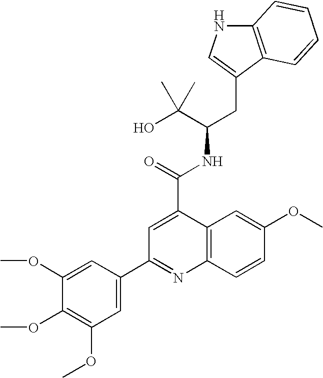 | | | |

### EXAMPLE 374

### 6-(4-Hydroxybut-1-ynyl)-2-(3,4,5-trimethoxyphenyl)quinoline-4-carboxylic acid [(R)-1-hydroxymethyl-2-(1H-indol-3-yl)ethyl]amide

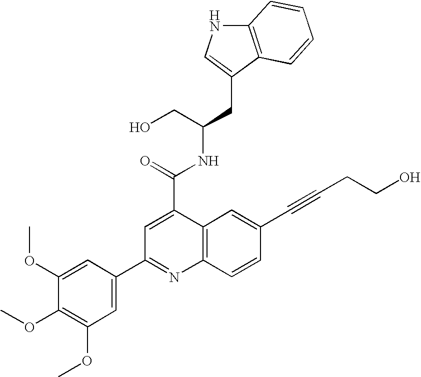

### 374a) 6-Iodo-2-(3,4,5-trimethoxyphenyl)quinoline-4-carboxylic acid

The title compound was obtained in analogy to general methods 13a. MS (ESI,+): 466 (M+1).

### 374b) Methyl 6-iodo-2-(3,4,5-trimethoxyphenyl)quinoline-4-carboxylate

The carboxylic acid (1 g) was dissolved in methanol (100 ml) and acidified with a few drops of conc. sulphuric acid. The mixture was stirred at room temperature overnight, and the title compound was precipitated by addition of water, filtered off and washed with water, and the residue was dried in vacuo. Yield 910 mg. MS (ESI,+): 480 (M+1).

### 374c) Methyl 6-(4-hydroxybut-1-ynyl)-2-(3,4,5-trimethoxyphenyl)quinoline-4-carboxyate

Methyl 6-iodo-2-(3,4,5-trimethoxyphenyl)quinoline-4-carboxylate (1 g), but-3-yn-1-ol (0.32 ml), CuI (397 mg), Pd(PPh₃)₄ (241 mg) and triethylamine (2.89 ml) were suspended in THF (20 ml) and stirred together at room temperature overnight. The mixture was added to water and extracted with ethyl acetate. The combined organic phases were dried over sodium sulphate and freed of solvent in a rotary evaporator. The title compound was obtained after flash chromatography in 41% yield (360 mg). MS (ESI,+): 422 (M+1).

### 374d) 6-(4-Hydroxybut-1-ynyl)-2-(3,4,5-trimethoxyphenyl)quinoline-4-carboxylic acid

The title compound was obtained in analogy to general method 39b.

### 374e) 6-(4-Hydroxybut-1-ynyl)-2-(3,4,5-trimethoxyphenyl)quinoline-4-carboxylic acid [(R)-1-hydroxymethyl-2-(1H-indol-3-yl)ethyl]amide

The title compound was obtained by reaction with (D)-tryptophanol in analogy to general method 113b.

(DMSO-d₆): 10.79 s (1H); 8.70 d (J=8.3 Hz, 1H); 8.01 m (3H); 7.65 m (2H); 7.50 s (2H); 7.32 d (J=7.8 Hz, 1H); 7.20 s (1H); 7.02 m (1H); 6.93 m (1H); 4.31 m (1H); 3.89 s (6H); 3.73 s (3H); 3.60 m (4H); 3.03 m (1H); 2.95 m (1H); 2.60 m (2H).

The following compounds were obtained in analogy to the preparation methods described in detail:

| | | Method | | |
|---|---|---|---|---|
| | | analo- | | |
| | Product; | gous | ¹H-NMR (400 MHz) δ | |
| Ex. | reagents | to | [ppm] | Structure |
| 375 | 6-(5-Hydroxypent-1-ynyl)- 2-(3,4,5-trimethoxy- phenyl)quinoline-4- carboxylic acid [(R)-1- hydroxymethyl-2-(1H- indol-3-yl)ethyl]amide; 6-Iodo-2-(3,4,5-trimethoxy- phenyl)quinoline-4- carboxylic acid [(R)-1- hydroxymethyl-2-(1H- indol-3-yl)ethyl]amide and Pent-4-yn-1-ol | 374 | (DMSO-d₆): 10.79 s (1H); | |
| # 8.70 d (J=8.3 Hz, 1H); 8.07 s (1H); 8.00 m (2H); 7.69 m (2H); 7.50 s (2H); 7.31 d (J=8.1 Hz, 1H); 7.19 s (1H); 7.02 m (1H); 6.93 m (1H); 4.33 m (1H); 3.90 s (6H); 3.73 s (3H); 3.51 m (4H); 3.03 m (1H); 2.96 m (1H); 2.50 m (2H); 1.70 m (2H). | 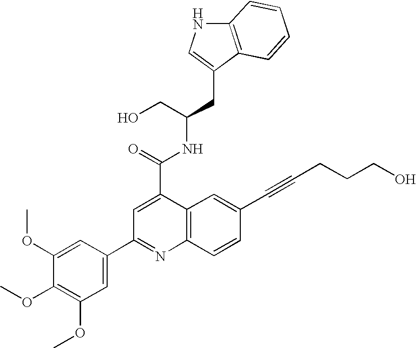 | | | |
| 376 | 6-(3-Hydroxyprop-1-ynyl)- 2-(3,4,5-trimethoxyphenyl) quinoline-4-carboxylic acid [(R)-1- hydroxymethyl-2-(1H- indol-3-yl)ethyl]amide; 6-Iodo-2-(3,4,5-trimethoxy- phenyl)quinoline-4- carboxylic acid [(R)-1- hydroxymethyl-2-(1H- indol-3-yl)ethyl]amide and Prop-2-yn-1-ol) | 374 | (DMSO-d₆): 10.78 s (1H); | |
| # 8.72 d (J=8.3 Hz, 1H); 8.11 s (1H); 8.02 m (2H); 7.72 d (J=2.0 Hz/8.8 Hz, 1H); 7.64 d (J=7.8 Hz, 1H); 7.51 s (2H); 7.31 d (J=8.1 Hz, 1H); 7.20 s (1H); 7.02 m (1H); 6.93 m (1H); 4.35 m (3H); 3.90 s (6H); 3.73 s (3H); 3.55 m (2H); 3.04 m (1H); 2.94 m (1H). | 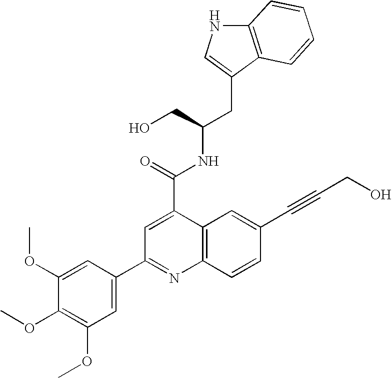 | | | |
| 377 | 6-(3-Methoxyprop-1-ynyl)- 2-(3,4,5-trimethoxyphenyl) quinoline-4-carboxylic acid [(R)-1-hydroxymethyl-2- (1H-indol-3-yl)ethyl]amide; 6-Iodo-2-(3,4,5-trimethoxy- phenyl)quinoline-4- carboxylic acid [(R)-1- hydroxymethyl-2-(1H- indol-3-yl)ethyl]amide and 3-Methoxypropyne | 374 | (DMSO-d₆): 10.78 s (1H); | |
| # 8.72 d (J=8.3 Hz, 1H); 8.13 s (1H); 8.03 m (2H); 7.76 d (J=1.8 Hz, 1H); 7.73 d (J=1.8 Hz, 1H); 7.52 s (2H); 7.30 d (J=8.1 Hz, 1H); 7.19 d (J=2.3 Hz, 1H); 7.02 m (1H); 6.92 m (1H); 4.37 s (2H); 4.33 m (1H); 3.90 s (6H); 3.73 s (3H); 3.57 s (3H); 3.59 m (2H); 3.02 m (1H); 2.95 m (1H). | 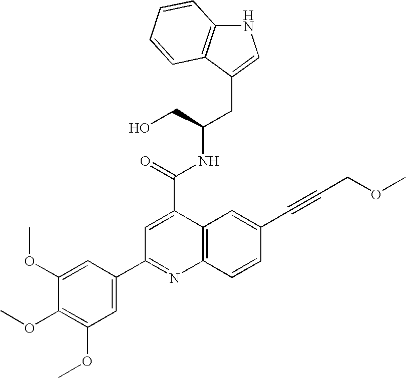 | | | |
| 378 | 5-(4-Hydroxybut-1-ynyl)- 3′,4′,5′-trimethoxybiphenyl- 3-carboxylic acid [(R)-1- hydroxymethyl-2-(1H- indol-3-yl)ethyl]amide; (D)-Tryptophanol and 5-(3-Hydroxybut-1-ynyl)- 3′,4′,5′-trimethoxybiphenyl- 3-carboxylic acid | 39a-b/ 374c-e | (DMSO-d₆): 10.75 s (1H); | |
| # 8.35 d (J=7.8 Hz, 1H); 7.97 s (1H); 7.82 s (1H); 7.80 s (1H); 7.65 d (J=7.8 Hz, 1H); 7.30 d (J=7.8 Hz, 1H); 7.13 s (1H); 7.03 t (J=7.4 Hz, 1H); 6.94 s (2H); 4.95 t (J=5.5 Hz, 1H); 4.80 t (J=5.5 Hz, 1H); 4.24 m (1H); 3.88 s (6H); 3.70 s (3H); 3.62 m (2H); 3.54 m (1H); 3.49 m (1H); 2.97 m (2H); 2.80 t (J=6.6 Hz, 2H). | 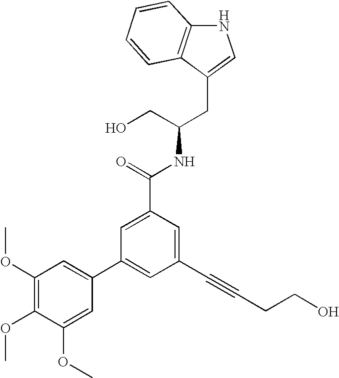 | | | |
| 379 | 5-(3-Hydroxyprop-1-ynyl)- 3′,4′,5′-trimethoxybiphenyl- 3-carboxylic acid [(R)-1- hydroxy-methyl-2-(1H- indol-3-yl)ethyl]amide; (D)-Tryptophanol and 5-(3-Hydroxyprop-1-ynyl)- 3′,4′,5′-trimethoxybiphenyl- 3-carboxylic acid | 39a-b/ 374c-e | (DMSO-d₆): 10.76 s (1H); 8.38 d (J=8.2 Hz, 1H); 8.02 s (1H); | |
| # 7.86 s (1H); 7.84 s (1H); 7.65 d (J=7.8 Hz, 1H); 7.30 d (J=8.2 Hz, 1H); 7.14 s (1H); 7.04 t (J=7 Hz, 1H); 6.94-6.97 m (3H); 5.40 t (J=5.9 Hz, 1H); 4.81 t (J=5.5 Hz, 1H); 4.35 d (J=5.5 Hz, 2H); 4.20-4.28 m (1H); 3.88 s (6H); 3.70 s (3H); 3.46-3.59 m (2H); 3.03 dd (J=14.4 Hz, J=7.8 Hz, 1H); 2.96 dd (J=14.4 Hz; J=7.8 Hz, 1H). | 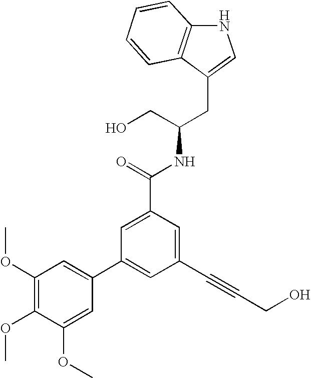 | | | |
| 380 | 5-(5-Hydroxypent-1-ynyl)- 3′,4′,5′-trimethoxybiphenyl- 3-carboxylic acid [(R)-1- hydroxy-methyl-2-(1H- indol-3-yl)ethyl]amide; (D)-Tryptophanol and 5-(5-Hydroxypent-1-ynyl)- 3′,4′,5′-trimethoxybiphenyl- 3-carboxylic acid | 39a-b/ 374c-e | (DMSO-d₆): 10.76 s (1H); 8.36 d (J=8.2 Hz, 1H); 7.97 s (1H); 7.80 d (J=4.3 Hz, 1H); | |
| # 7.66 d (J=7.8 Hz, 1H); 7.30 d (J=7.8 Hz, 1H); 7.14 s (1H); 7.04 t (J=7.4 Hz, 1H); 6.94-6.97 m (3H); 4.81 t (1H); 4.58 t (J=3.9 Hz, 1H); 4.20-4.28 m (1H); 3.88 s (6H); 3.70 s (3H); 3.46-3.58 m (4H); 3.02 dd (J=14.4 Hz, J=7.8 Hz, 1H); 2.93 dd (J=14.4 Hz, J=7.8 Hz, 1H); 2.49-2.53 m (2H); 1.72 q (J=6.6 Hz, 2H). | 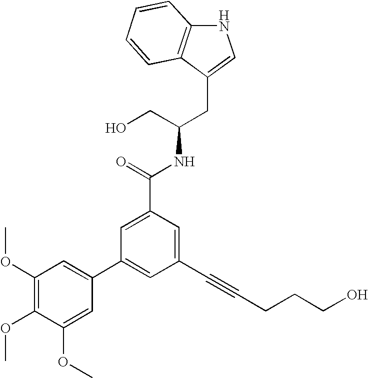 | | | |
| 381 | 3′,4′,5′-Trimethoxy-5-(3- methoxyprop-1-ynyl)- biphenyl-3-carboxylic acid [(R)-1-hydroxy-methyl-2- (1H-indol-3-yl)ethyl]amide; (D)-Tryptophanol and 3′,4′,5′Trimethoxy-5-(3- methoxyprop-1-ynyl) biphenyl-3-carboxylic acid | 39a-b/ 374c-e | (DMSO-d₆): 10.76 s (1H); 8.36 d (J=8.2 Hz, 1H); 8.04 s | |
| # (1H); 7.89 (s, 2H); 7.66 d (J=7.8 Hz, 1H); 7.30 d (J=8.2 Hz); 7.14 s (1H); 7.04 t (J=7.4 Hz, 1H); 6.94-6.96 m (3H); 4.81 t (J=5.9 Hz, 1H); 4.36 s (2H); 4.52 m (1H); 3.88 s (6H); 3.70 s (3H); 3.47-3.59 m (2H); 3.37 s (3H); 3.03 dd (J=14.4 Hz, J=5.8 Hz, 1H); 2.94 dd (J=14.4 Hz, J=7.8 Hz, 1H). | 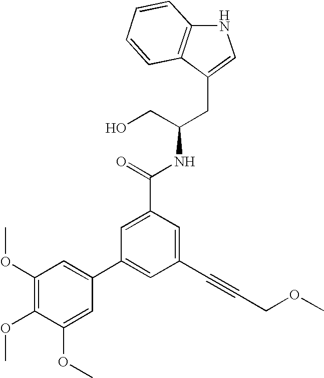 | | | |
| 382 | 3′,4′-Dimethoxy-5-(3- methoxyprop-1-ynyl) biphenyl-3-carboxylic acid [(R)-1-hydroxy-methyl-2- (1H-indol-3-yl)ethyl]amide; (D)-Tryptophanol and 3′,4′-Dimethoxy-5-(3- methoxyprop-1-ynyl)b/ phenyl-3-carboxylic acid | 39a-b/ 374c-e | (DMSO-d₆): 10.76 s (1H); 8.39 d (J=8.2 Hz, 1H); 8.04 s (1H); | |
| # 7.85 s (2H); 7.66 d (J=7.8 Hz, 1H); 7.26-7.31 m (3H); 7.14 (1H); 7.02-7.07 m (2H); 6.96 t (J=7.42; 1H); 4.82 t (J=5.8 Hz, 1H); 4.37 s (2H); 4.21-4.29 m (1H); 3.87 s (3H); 3.80 s (3H); 3.46-3.58 m (2H); 3.37 s (3H); 3.03 dd (J=14.4 Hz; J=5.8 Hz, 1H); 2.93 dd (J=14.4 Hz, J=7.8 Hz, 1H). | 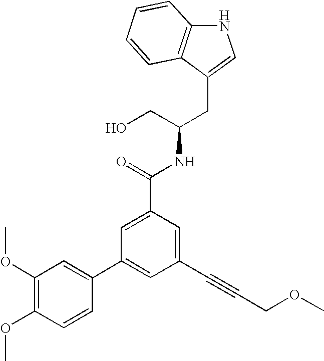 | | | |
| 383 | 5-(3-Hydroxyprop-1-ynyl)- 3′,4′-dimethoxybiphenyl-3- carboxylic acid [(R)-1- hydroxymethyl-2-(1H- indol-3-yl)ethyl]amide; (D)-Tryptophanol and 5-(3-Hydroxyprop-1-ynyl)- 3′,4′-dimethoxybiphenyl- 3-carboxylic acid | 39a-b/ 374c-e | (DMSO-d₆): 10.76 s (1H); 8.38 d (J=8.2 Hz, 1H); 8.02 s (1H); 7.83 s (1H); 7.80 s (1H); 7.65 d | |
| # (J=7.8 Hz, 1H); 7.30 d (J=8.2 Hz, 1H); 7.25-7.27 m (2H); 7.13 (1H); 7.02-7.07 m (2H); 6.96 t (J=7.42 Hz, 1H); 5.40 m (1H); 4.81 t (J=5.8 Hz, 1H); 4.35 m (2H); 4.21-4.29 m (1H); 3.86 s (3H); 3.80 s (3H); 3.46-3.58 m (2H); 3.03 dd (J=14.5 Hz, J=6 Hz, 1H); 2.93 dd (J=14.5 Hz, J=7.8 Hz, 1H). | 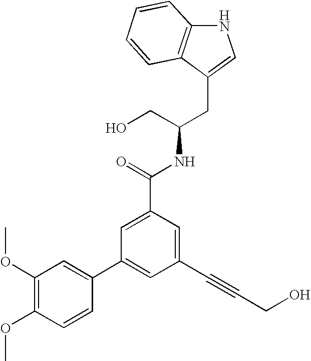 | | | |
| 384 | 3′,4′,5′-Trimethoxy-5-(4- methoxyphenylethynyl) biphenyl-3-carboxylic acid [(R)-1-hydroxymethyl-2- (1H-indol-3-yl)ethyl]amide; (D)-Tryptophanol and 3′,4′,5′-Trimethoxy-5-(4- methoxyphenylethynyl) biphenyl-3-carboxylic acid | 39a-b/ 374c-e | (DMSO-d₆): 10.77 s (1H); 8.40 d (J=7.8 Hz, 1H); 8.03 s (1H); 7.95 s (2H); 7.67 d | |
| # (J=7.8 Hz, 1H); 7.55 s (1H); 7.53 s (1H); 7.31 (J=8.2 Hz, 1H); 7.15 (1H); 6.94-7.06 m (6H); 4.83 t (J=5.5 Hz, 1H); 4.22-4.30 m (1H); 3.89 s (6H); 3.80 s (3H); 3.71 s (3H); 3.48-3.60 m (2H); 3.03 dd (J=14.8 Hz, J=6 Hz, 1H); 2.95 dd (J=14.4 Hz, J=7.4 Hz, 1H). | 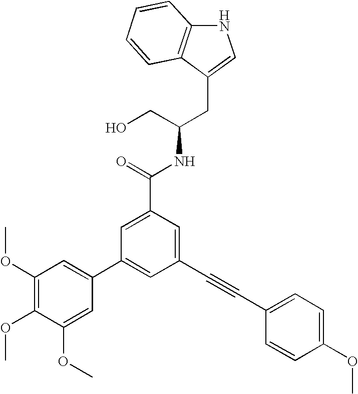 | | | |

### EXAMPLE 385

### 3′,4′,5′-Trimethoxy-5-((Z)-3-methoxypropenyl)biphenyl-3-carboxylic acid [(R)-1-hydroxymethyl-2-(1H-indol-3-yl)ethyl]amide

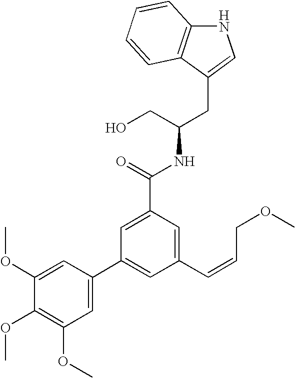

620 mg of zinc dust are suspended in 3.6 ml of water. Argon is passed through the vigorously stirred suspension for 15 min. Then 62 mg of copper(II) acetate are added, and the mixture is stirred for 15 min. Subsequently 62 mg of sliver nitrate are added and stirring is continued for 30 min. The metal is filtered off with suction under argon. It is washed with 2×1.8 ml of water, 2×1.8 ml of methanol, 2×3.6 ml of acetone and 2×3.6 ml of diethyl ether.

The activated zinc obtained in this way is transferred while still moist with ether into a solution of 50 mg of 5-(3-hydroxyprop-1-ynyl)-3′,4′,5′-trimethoxybiphenyl-3-carboxylic acid
[(R)-1-hydroxymethyl-2-(1H-indol-3-yl)ethyl]amide (Example # 381) in 1.3 ml of methanol and 0.5 ml of water. The reaction mixture is stirred until the reaction is complete. The metal is filtered off with suction (caution: the remaining metal is pyrophoric), and washed with methanol, and the solvent is evaporated. The title compound is obtained as a colourless foam (45 mg, 89% of theory).

¹H-NMR (400 MHz) δ [ppm] (DMSO-d₆): 10.77 s (1H); 8.30 d (J=8.2 Hz, 1H); 7.93 s (1H); 7.66 d (J=7.8 Hz, 1H); 7.62 s (2H); 7.30 d (J=8.2 Hz, 1H); 7.15 s (1H); 7.03 t (J=7.4 Hz, 1H); 6.93-6.97 m (3H); 6.68 d (J=12.1 Hz, 1H); 5.88-5.94 m (1H); 4.82 t (J=5.6 Hz, 1H); 4.20-4.26 m (3H); 3.88 s (6H); 3.71 s (3H); 3.47-3.60 m (2H); 3.27 s (3H); 3.04 dd (J=14.5 Hz, J=5.8 Hz, 1H); 2.95 dd (J=14.4 Hz, J=7.8 Hz, 1H).

The following compounds were obtained in analogy to the preparation methods described in detail:

| | | Method | | |
|---|---|---|---|---|
| | Product; | analogous | ¹H-NMR (400 MHz) δ | |
| Ex. | reagents | to | [ppm] | Structure |
| 386 | 5-((Z)-4-Hydroxybut-1-enyl)- 3′,4′, 5′-trimethoxybiphenyl-3- carboxylic acid [(R)-1-hydroxy- methyl-2-(1H-indol-3- yl)ethyl]amide; 5-(4-Hydroxybut-1-ynyl)-3′,4′,5′- trimethoxybiphenyl-3-carboxylic acid [(R)-1-hydroxymethyl-2- (1H-indol-3-yl)ethyl]amide | 385 | (DMSO-d₆): 10.76 s (1H); | |
| # 8.27 d (J=8.2 Hz, 1H); 7.90 s (1H); 7.74 s (1H); 7.74 d (J=7.8 Hz, 1H), 7.70 s (1H); 7.66 (J=7.8 Hz, 1H); 7.30 d (J=8.2 Hz, 1H); 7.16 s (1H); 7.03 t (J=7.4 Hz, | | | | |
| # 1H); 6.95-6.97 m (3H); 6.58 d (J=11.7 Hz, 1H) 5.78-5.84 m (1H); 4.81 t (J=5.8 Hz, 1H); 4.69 t (J=4.7 Hz, 1H); 4.20-4.28 m (1H); 3.88 s (6H); 3.71 s (3H); 3.47-3.60 m (4H); 3.04 dd (J=14.8 Hz, J=6 Hz, 1H); 2.95 dd (J=14.4 Hz, J=7.8 Hz, 1H); 2.47-2.52 m (2H). | 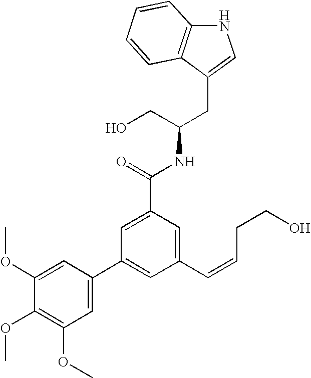 | | | |
| 387 | 5-((Z)-3-Hydroxypropenyl)- 3′,4′,5′-trimethoxybiphenyl-3- carboxylic acid [(R)-1-hydroxy- methyl-2-(1H-indol-3- yl)ethyl]amide; 5-(3-Hydroxyprop-1-ynyl)- 3′,4′,5′-trimethoxybiphenyl-3- carboxylic acid [(R)-1-hydroxy- methyl-2-(1H-indol-3- yl)ethyl]amide | 385 | (DMSO-d₆): 10.77 s (1H); 8.29 d | |
| # (J=7.8 Hz, 1H); 7.91 s (1H); 7.67 d (J=7.8 Hz, 1H); 7.64 s (1H); 7.61 s (1H); 7.30 (J=8.2 Hz, 1H); 7.16 (1H); 7.03 t (J=7.4 Hz, 1H); 6.94-6.97 m (3H); 6.58 d (J=11.7 Hz, 1H); 5.88-5.94 m (1H); 4.96 t (J=5.1 Hz, 1H); 4.82 t (J=5.1 Hz, | | | | |
| # 1H); 4.82 t (J=4.8 Hz, 1H); 4.21-4.30 m (3H); 3.88 s (6H); 3.71 s (3H); 3.47-3.60 m (2H); 3.27 s (3H); 3.04 dd (J=14.5 Hz, J=5.8 Hz, 1H); 2.95 dd (J=14.4 Hz, J=7.8 Hz, 1H). | 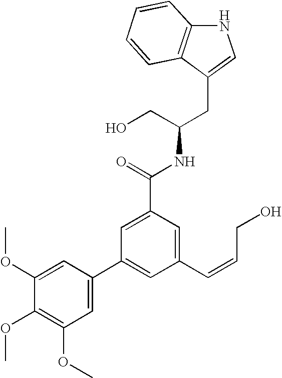 | | | |
| 388 | 5-((Z)-5-Hydroxypent-1-enyl)- 3′,4′,5′-trimethoxybiphenyl-3- carboxylic acid [(R)-1-hydroxy- methyl-2-(1H-indol-3- yl)ethyl]amide; 5-(5-Hydroxypent-1-ynyl)- 3′,4′,5′-trimethoxybiphenyl-3- carboxylic acid [(R)-1-hydroxy- methyl-2-(1H-indol-3- yl)ethyl]amide | 385 | (DMSO-d₆): 10.76 s (1H); 8.26 d | |
| # (J=8.2 Hz, 1H); 7.89 s (1H); 7.69 s (2H); 7.66 s (1H); 7.30 (J=8.2 Hz, 1H); 7.16 s (1H); 7.04 t (J=7.4 Hz, 1H); 6.94-6.97 m (3H); 6.52 d (J=11.3 Hz, 1H); 5.75-5.81 m (1H); 4.81 t (J=5.6 Hz, 1H); 4.51 t (J=5 Hz, 1H); 4.21-4.28 m (1H); 3.88 s (6H); 3.71 s (3H); 3.43-3.60 | | | | |
| # m (4H); 3.05 dd (J=14.5 Hz, J=5.8 Hz, 1H); 2.95 dd (J=14.4 Hz, J=7.8 Hz, 1H); 2.39 q (J=7.4 Hz, 2H); 1.58-1.65 m (2H). | 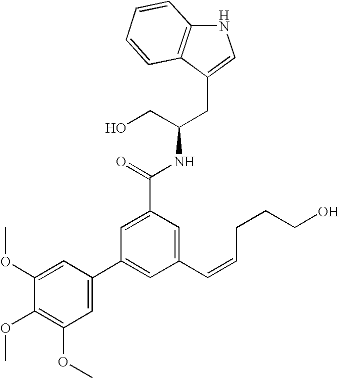 | | | |

### EXAMPLE 389

### 6-(5-Hydroxypentyl)-2-(3,4,5-trimethoxyphenyl)quinoline-4-carboxylic acid [(R)-1-hydroxymethyl-2-(1H-indol-3-yl)ethyl]amide;

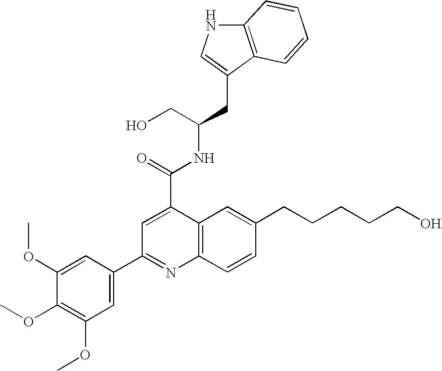

6-(5-Hydroxypent-1-ynyl)-2-(3,4,5-trimethoxyphenyl)quinoline-4-carboxylic acid [(R)-1-hydroxymethyl-2-(1H-indol-3-yl)ethyl]amide (100 mg) and palladium on carbon (10%, 50 mg) were suspended in methanol (10 ml) and hydrogenated under hydrogen at atmospheric pressure at room temperature. After hydrogen uptake ceased, the catalyst was filtered off and the mother liquor was stripped off in a rotary evaporator. Drying in vacuo resulted in the title compound in 42% yield (42 mg).

(DMSO-d₆): 10.79 s (1H); 8.60 d (J=8.3 Hz, 1H); 7.93 m (2H); 7.70 s (1H); 7.62 m (2H); 7.49 s (2H); 7.32 d (J=8.1 Hz, 1H); 7.19 s (1H); 7.03 m (1H); 6.93 m (1H); 4.85 m (1H); 4.32 m (1H); 3.89 s (6H); 3.72 s (3H); 3.56 m (2H); 3.36 m (2H); 3.05 m (1H); 2.93 m (1H); 2.63 m (2H); 1.55 m (2H); 1.39 m (2H); 1.30 m (2H).

The following compounds were obtained in analogy to the preparation methods described in detail:

| | | Method | | |
|---|---|---|---|---|
| | | analo- | | |
| | Product; | gous | ¹H-NMR (400 MHz) δ | |
| Ex. | reagents | to | [ppm] | Structure |
| 390 | 6-(4-Hydroxybutyl)-2- (3,4,5-trimethoxyphenyl) quinoline-4-carboxylic acid [(R)-1-hydroxy-methyl-2- (1H-indol-3-yl)ethyl]amide; 6-(4-Hydroxybut-1-ynyl)-2- (3,4,5-trimethoxyphenyl) quinoline-4-carboxylic acid [(R)-1-hydroxymethyl-2- (1H-indol-3-yl)ethyl]amide | 389 | (DMSO-d₆): 10.79 s (1H); | |
| # 8.59 d (J=8.5 Hz, 1H); 7.97 d (J=8.5 Hz, 1H); 7.94 s (1H); 7.71 s (1H); 7.62 m (2H); 7.49 s (2H); 7.31 d (J=8.1 Hz, 1H); 7.19 s (1H); 7.04 m (1H); 6.93 m (1H); 4.85 m (1H); 4.40 m (2H); 3.89 s (6H); 3.72 s (3H); 3.56 m (2H); 3.38 m (2H); 3.03 m (1H); 2.94 m (1H); 2.64 m (2H); 1.59 m (2H); 1.43 m (2H). | 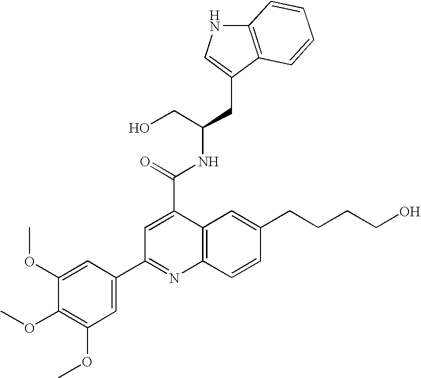 | | | |
| 391 | 6-(3-Hydroxypropyl)-2- (3,4,5-trimethoxyphenyl) quinoline-4-carboxylic acid [(R)-1-hydroxy-methyl-2- (1H-indol-3-yl)ethyl]amide; 6-(3-Hydroxyprop-1-ynyl)- 2-(3,4,5-trimethoxyphenyl) quinoline-4-carboxylic acid [(R)-1-hydroxymethyl-2- (1H-indol-3-yl)ethyl]amide | 389 | (DMSO-d₆): 10.83 s (1H); | |
| # 8.64 d (J=8.5 Hz, 1H); 8.00 d (J=8.7 Hz, 1H); 7.97 s (1H); 7.76 d (J=1.5 Hz, 1H); 7.66 m (2H); 7.53 s (2H); 7.35 d (J=7.9 Hz, 1H); 7.23 s | | | | |
| # (1H); 7.06 m (1H); 6.97 m (1H); 4.91 m (1H); 4.59 m (1H); 4.40 m (1H); 3.93 s (6H); 3.76 s (3H); 3.59 m (2H); 3.45 m (2H); 3.06 dd (J=5.8 Hz/14.9 Hz, 1H); 2.96 dd (J=8.3 Hz/14.7 Hz, 1H); 2.73 m (2H); 1.76 m (2H). | 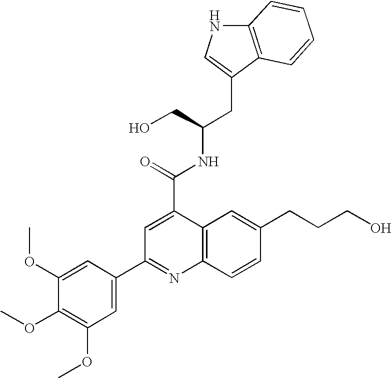 | | | |
| 392 | 6-(3-Methoxypropyl)-2- (3,4,5-trimethoxyphenyl) quinoline-4-carboxylic acid [(R)-1-hydroxy-methyl-2- (1H-indol-3-yl)ethyl]amide; 6-(3-Methoxyprop-1-ynyl)- 2-(3,4,5-trimethoxyphenyl) quinoline-4-carboxylic acid [(R)-1-hydroxymethyl-2- (1H-indol-3-yl)ethyl]amide | 389 | (DMSO-d₆): 10.78 s (1H); | |
| # 8.61 d (J=8.3 Hz, 1H); 7.97 d (J=8.7 Hz, 1H); 7.94 s (1H); 7.72 s (1H); 7.62 m (2H); 7.49 s (2H); 7.31 d (J=8.1 Hz, 1H); 7.19 s (1H); 7.03 m (1H); 6.93 m (1H); 4.85 m (1H); 4.36 m (1H); 3.89 s (6H); 3.72 s (3H); 3.56 m (2H); 3.31 m (2H); 3.21 s (3H); 3.03 m (1H); 2.92 m (1H); 2.68 m (2H); 1.77 m (2H). | 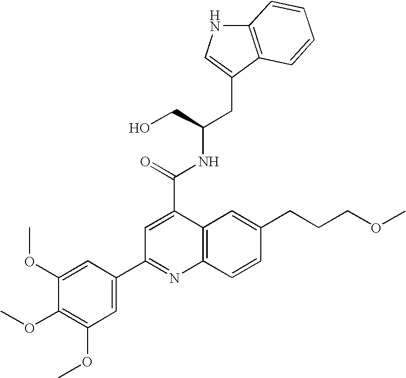 | | | |
| 393 | 3′,4′,5′-Trimethoxy-4-(3- methoxypropyl)biphenyl-3- carboxylic acid [(R)-1- hydroxy-methyl-2-(1H- indol-3-yl)ethyl]amide; 3′,4′,5′-Trimethoxy-4-(3- methoxyprop-1-ynyl) biphenyl-3-carboxylic acid [(R)-1-hydroxy-methyl-2- (1H-indol-3-yl)ethyl]amide | 389 | (DMSO-d₆): 10.79 s (1H); | |
| # 8.17 d (J=8.2 Hz, 1H); 7.63 d (J=7.8 Hz, 1H); 7.58 7.61 m (1H); 7.43 s (1H); 7.31 d (J=7.8 Hz, 1H); 7.26 d (J=7.8 Hz, 1H); 7.17 s (1H); 7.03 t (J=7.4 Hz, 1H); 6.94 t (J=7.4 Hz, 1H); 6.87 s (2H); 4.80 t (J=5.6 | | | | |
| # Hz, 1H); 4.18-4.26 m (1H); 3.85 s (6H); 3.70 s (3H); 3.51-3.58 m (1H); 3.42-3.48 m (1H); 3.19-3.22 m (5H); 3.03 dd (J=14.5 Hz, J=5.8 Hz, 1H); 2.86 dd (J=14.4 Hz, J=8.6 Hz, 1H); 2.62-2.70 m (2H); 1.66-1.73 m (2H). | 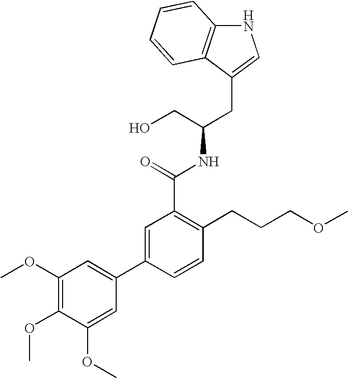 | | | |
| 394 | 3′,4′,5′-Trimethoxy-5-(3- methoxypropyl)biphenyl-3- carboxylic acid [(R)-1- hydroxy-methyl-2-(1H- indol-3-yl)ethyl]amide; 3′, 4′, 5′-Trimethoxy-5-(3- methoxyprop-1-ynyl) biphenyl-3-carboxylic acid [(R)-1-hydroxy-methyl-2- (1H-indol-3-yl)ethyl]amide | 389 | (DMSO-d₆): 10.76 s (1H); | |
| # 8.21 d (J=8.2 Hz, 1H); 7.84 s (1H); 7.67 d (J=7.8 Hz, 1H); 7.62 s (2H); 7.30 d (J=7.8 Hz, 1H); 7.15 s (1H); 7.03 t (J=7.4 Hz, 1H); 6.92-6.97 m (3H); 4.81 t (J=5.6 Hz, 1H); | | | | |
| # 4.20-4.28 m (1H); 3.88 s (6H); 3.70 s (3H); 3.46-3.59 m (2H); 3.36 t (J=6.2 Hz, 2H); 3.25 s (3H); 3.04 dd (J=14.5 Hz, J=5.8 Hz, 1H); 2.94 dd (J=14.4 Hz, J=8.3 Hz, 1H); 2.72 t (J=7.6 Hz, 2H); 1.83-1.91 m (2H). | 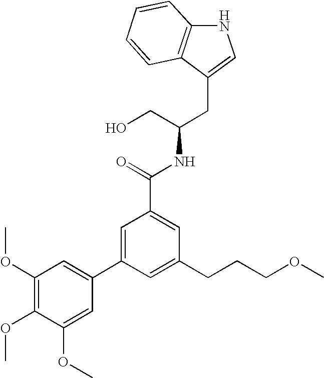 | | | |
| 395 | 3′,4′-Dimethoxy-5-(3- methoxypropyl)biphenyl-3- carboxylic acid [(R)-1- hydroxymethyl-2-(1H- indol-3-yl)ethyl]amide; 3′,4′-Dimethoxy-5-(3- methoxyprop-1-ynyl) biphenyl-3-carboxylic acid [(R)-1-hydroxymethyl-2- (1H-indol-3-yl)ethyl]amide | 389 | (DMSO-d₆): 10.76 s (1H); | |
| # 8.21 d (J=8.2 Hz, 1H); 7.84 s (1H); 7.67 d (J=7.8 Hz, 1H); 7.59 s (2H); 7.30 d (J=7.8 Hz, 1H); 7.22-7.24 m (2H); 7.14 (1H); 7.02-7.06 m (2H); 6.96 t (J=7.4 Hz, 1H); 4.81 t (J=5.6 Hz, 1H); 4.21-4.29 m (1H); | | | | |
| # 3.86 s (3H); 3.80 s (3H); 3.46-3.59 m (2H); 3.35 t (J=6.2 Hz, 2H); 3.25 s (3H); 3.04 dd (J=14.5 Hz, J=5.8 Hz, 1H); 2.94 dd (J=14.4 Hz, J=7.8 Hz, 1H); 2.71 t (J=7.8 Hz, 2H); 1.83-1.90 m (2H). | 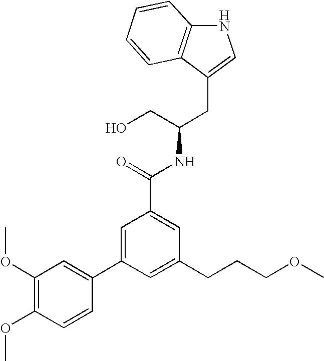 | | | |
| 396 | 5-(3-Hydroxypropyl)-3′,4′- dimethoxybiphenyl-3- carboxylic acid [(R)-1- hydroxymethyl-2-(1H- indol-3-yl)ethyl]amide; 5- (3-Hydroxypropyl)-3′,4′- dimethoxybiphenyl-3- carboxylic acid [(R)-1- hydroxymethyl-2-(1H- indol-3-yl)ethyl]amide | 389 | (DMSO-d₆): 10.75 s (1H); | |
| # 8.21 d (J=8.2 Hz, 1H); 7.83 s (1H); 7.67 d (J=7.4 Hz, 1H); 7.59 s (2H); 7.30 d (J=7.8 Hz, 1H); 7.22-7.24 m (2H); 7.14 (1H); 7.02-7.06 m (2H); 6.96 t (J=7.4 Hz, 1H); 4.80 t (J=5.6 | | | | |
| # Hz, 1H); 4.52 (J=5.1 Hz; 1H); 4.21-4.29 m (1H); 3.86 s (3H); 3.80 s (3H); 3.42-3.59 m (4H); 3.04 dd (J=14.5 Hz, J=5.8 Hz, 1H); 2.94 dd (J=14.4 Hz, J=7.8 Hz, 1H); 2.71 t (J=7.6 Hz, 2H); 1.75-1.82 m (2H). | 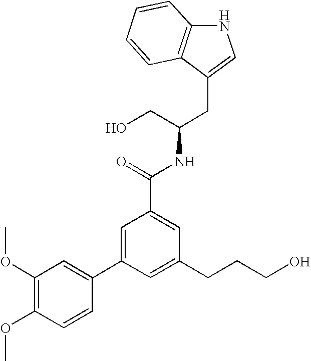 | | | |
| 397 | 5-(5-Hydroxypentyl)- 3′,4′,5′-trimethoxybiphenyl- 3-carboxylic acid [(R)-1- hydroxymethyl-2-(1H- indol-3-yl)ethyl]amide; 5- (5-Hydroxypent-1-ynyl)- 3′,4′,5′-trimethoxybiphenyl- 3-carboxylic acid [(R)-1- hydroxy-methyl-2-(1H- indol-3-yl)ethyl]amide | 389 | (DMSO-d₆): 10.76 s (1H); | |
| # 8.20 d (J=7.8 Hz, 1H); 7.83 s (1H); 7.67 d (J=7.8 Hz, 1H); 7.60 s (2H); 7.30 d (J=7.8 Hz, 1H); 7.15 s (1H); 7.03 t (J=7.4 Hz, 1H); 6.95 t (J=7.4 Hz, 1H); 6.92 s (2H); 4.81 t (J=4.7 Hz, 1H); 4.37 t (J=4.5 Hz, 1H); 4.20-4.29 m (1H); 3.88 s (6H); | | | | |
| # 3.70 s (3H); 3.47-3.59 m (2H); 3.37-3.41 m (2H); 3.04 dd (J=14.5 Hz, J=5.8 Hz, 1H); 2.94 dd (J=14.4 Hz, J=7.8 Hz, 1H); 2.68 t (J=7.6 Hz, 2H); 1.60-1.67 m (2H); 1.44-1.51 m (2H); 1.31-1.38 m (2H). | 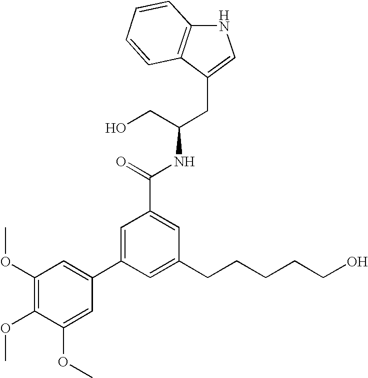 | | | |
| 398 | 5-(3-Hydroxypropyl)- 3′,4′,5′-trimethoxybiphenyl- 3-carboxylic acid [(R)-1- hydroxymethyl-2-(1H- indol-3-yl)ethyl]amide; 5-(3-Hydroxypropyl)- 3′,4′,5′-trimethoxybiphenyl- 3-carboxylic acid [(R)-1- hydroxymethyl-2-(1H- indol-3-yl)ethyl)amide | 389 | (DMSO-d₆): 10.76 s (1H); | |
| # 8.21 d (J=8.2 Hz, 1H); 7.83 s (1H); 7.67 d (J=7.8 Hz, 1H); 7.62 (2H); 7.30 d (J=7.8 Hz, 1H); 7.14 s (1H); 7.03 t (J=7.4 Hz, 1H); 6.95 t (J=7.4 | | | | |
| # Hz, 1H); 6.92 s (2H); 4.81 t (J=5.4 Hz, 1H); 4.53 t (J=5 Hz, 1H); 4.20-4.28 m (1H); 3.88 s (6H); 3.70 s (3H); 3.43-3.59 m (4H); 3.03 dd (J=14.5 Hz, J=5.8 Hz, 1H); 2.94 dd (J=14.4 Hz, J=7.8 Hz, 1H); 2.72 t (J=7.6 Hz, 2H); 1.76-1.83 m (2H). | 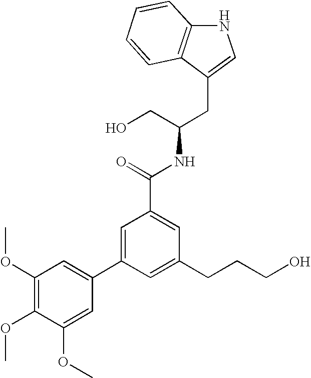 | | | |
| 399 | 5-(4-Hydroxybutyl)-3′,4′,5′- trimethoxybiphenyl-3- carboxylic acid [(R)-1- hydroxymethyl-2-(1H- indol-3-yl)ethyl]amide; 5-(4-Hydroxybut-1-ynyl)- 3′,4′,5′-trimethoxybiphenyl- 3-carboxylic trimethoxy- biphenyl-3-carboxylic acid [(R)-1-hydroxymethyl-2- (1H-indol-3-yl)ethyl]amide | 389 | (DMSO-d₆): 10.76 s (1H); | |
| # 8.20 d (J=8.2 Hz, 1H); 7.84 s (1H); 7.67 d (J=7.7 Hz, 1H); 7.61 s (2H); 7.30 d (J=7.8 Hz, 1H); 7.15 s (1H); 7.03 t (J=7.4 Hz, 1H); 6.95 t (J=7.4 Hz, 1H); 6.92 s (2H); 4.81 t (J=5.5 Hz, 1H); 4.41 t (J=5.1 Hz, 1H); | | | | |
| # 4.20-4.28 m (1H); 3.88 s (6H); 3.70 s (3H); 3.46-3.60 m (2H); 3.41-3.46 m (2H); 3.04 dd (J=14.5 Hz, J=5.8 Hz, 1H); 2.94 dd (J=14.4 Hz, J=7.8 Hz, 1H); 2.68 t (J=7.6 Hz, 2H); 1.63-1.70 m (2H), 1.44-1.51 m (2H). | 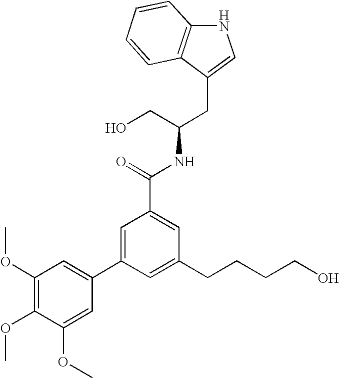 | | | |
| 400 | 3′,4′,5′-Trimethoxy-5-[2-(4- methoxyphenyl)ethyl]- biphenyl-3-carboxylic acid [(R)-1-hydroxymethyl-2- (1H-indol-3-yl)ethyl]amide; 3′,4′,5′-Trimethoxy-5-(4- methoxyphenylethynyl) biphenyl-3-carboxylic acid [(R)-1-hydroxymethyl-2- (1H-indol-3-yl)ethyl]amide | 389 | (DMSO-d₆): 10.78 s (1H); | |
| # 8.22 d (J=8.2 Hz, 1H); 7.84 s (1H); 7.70-7.67 (2H); 7.53 s (1H); 7.31 d (J=7.8 Hz, 1H); 7.15-7.17 m (3H); 7.04 t (J=7.4 Hz, 1H); 6.96 t (J=7.4 Hz, 1H); 6.83-6.86 m (4H); 4.83 t (J=5.5 Hz, 1H); 4.21-4.29 m (1H); 3.87 s (3H); 3.70 s (6H); 3.48-3.60 m (2H); 3.86-3.07 m (6H). | 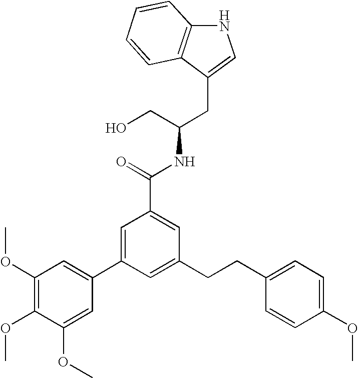 | | | |
| 401 | N-[(R)-2-[1-(2- Cyanoethyl)-1H-indol-3- yl]-1-(hydroxymethyl) ethyl]-3′-fluoro-4′- methoxy[1,1′-biphenyl]-3- carboxamide; 1-(2- Cyanoethyl)-L-tryptophanol and 3′-Fluoro-4′- methoxy[1,1′-biphenyl]-3- carboxylic acid | 39 | (DMSO-d₆): 8.31 d (J=8.1 | |
| # Hz, 1H); 8.05 s (1H); 7.78 d (J=7.8 Hz, 1H); 7.78 d (J=7.6 Hz, 1H); 7.73 d (J=8.0 Hz, 1H); 7.65 dd (J=12.9 Hz/2.3 Hz, 1H); 7.53 d (J=8.8 Hz, 1H); 7.51 d (J=8.0 Hz, 1H); 7.50 dd (J=7.8 Hz/7.6 Hz, 1H); 7.29 dd (J=9.1 Hz/8.8 Hz, 1H); | | | | |
| # 7.26 s (1H); 7.13 dd (J=8.0 Hz/7.0 Hz, 1H); 7.03 dd (J=8.0 Hz/7.0 Hz, 1H); 4.81 m (1H); 4.42 t (J=6.6 Hz, 2H); 4.25 m (1H); 3.90 s (3H); 3.54 m (1H); 3.50 m (1H); 3.03 dd (J=14.4 Hz/6.6 Hz, 1H); 2.95 t (J=6.6 Hz, 2H); 2.94 m (1H). | 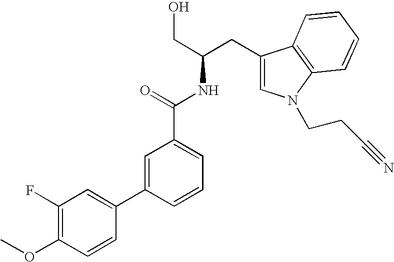 | | | |
| 402 | 3′-Fluoro-N-[(R)-2-(1- heptyl-1H-indol-3-yl)-1- (hydroxymethyl)ethyl]-4′- methoxy[1,1′-biphenyl]-3- carboxamide; 1-Heptyl-L- tryptophanol and 3′-Fluoro- 4′-methoxy[1,1′-biphenyl]- 3-carboxylic acid | 39 | (DMSO-d₆): 8.27 d (J=8.1 | |
| # Hz, 1H); 8.03 s (1H); 7.78 d (J=7.8 Hz, 1H); 7.76 d (J=7.8 Hz, 1H); 7.66 d (J=8.0 Hz, 1H); 7.64 dd (J=12.9 Hz/2.3 Hz, 1H); 7.53 d (J=8.8 Hz, 1H); 7.49 dd (J=7.8 Hz/7.8 Hz, 1H); 7.36 d (J=8.0 Hz, 1H); 7.28 dd (J=8.8 Hz/8.8 Hz, 1H); | | | | |
| # 7.17 s (1H); 7.08 dd (J=8.0 Hz/7.0 Hz, 1H); 6.98 dd (J=8.0 Hz/7.0 Hz, 1H); 4.82 m (1H); 4.25 m (1H); 4.05 t (J=6.9 Hz, 2H); 3.89 s (3H); 3.56 m (1H); 3.51 m (1H); 3.04 dd (J=14.4 Hz/5.8 Hz, 1H); 2.93 dd (J=14.4 Hz/8.1 Hz, 1H); 1.61 m (2H); 1.09 m (8H); 0.76 t (J=7.1 Hz, 3H). | 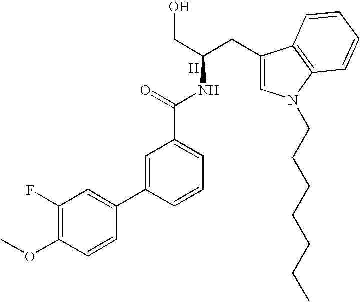 | | | |
| 403 | N-[(R)-2-[1-(4- Cyanobutyl)-1H-indol-3- yl]-1-(hydroxymethyl) ethyl]-3′-fluoro-4′- methoxy[1,1′-biphenyl]-3- carboxamide; 1-(4- Cyanobutyl)-L-tryptophanol and 3′-Fluoro-4′- methoxy[1,1′-biphenyl]-3- carboxylic acid | 39 | (DMSO-d₆): 8.27 d | |
| # (J=8.1 Hz, 1H); 8.04 s (1H); 7.78 d (J=7.7 Hz, 1H); 7.77 d (J=7.5 Hz, 1H); 7.68 d (J=8.0 Hz, 1H); 7.65 dd (J=13.0 Hz/2.3 Hz, 1H); 7.54 d (J=8.7 Hz, 1H); 7.50 dd (J=7.7 Hz/7.5 Hz, 1H); 7.41 d (J=8.0 Hz, 1H); 7.28 dd (J=9.0 Hz/8.7 Hz, 1H); 7.18 s (1H); 7.10 dd (J=8.0 Hz/7.0 | | | | |
| # Hz, 1H); 7.00 dd (J=8.0 Hz/7.0 Hz, 1H); 4.81 m (1H); 4.13 t (J=6.8 Hz, 2H); 4.25 m (1H); 3.90 s (3H); 3.55 m (1H); 3.52 m (1H); 3.03 dd (J=14.6 Hz/6.2 Hz, 1H); 2.96 t (J=7.1 Hz, 2H); 2.93 dd (J=14.6 Hz/7.4 Hz, 1H); 1.74 m (2H); 1.40 m (2H). | 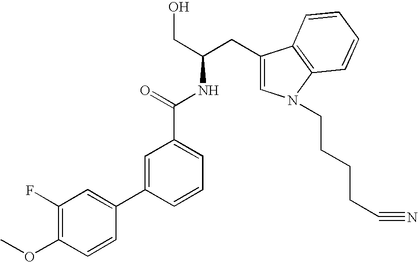 | | | |
| 404 | 3′-Fluoro-N-[(R)-1- (hydroxymethyl)-2-[1-(3- phenoxypropyl)-1H-indol- 3-yl]ethyl]-4′-methoxy[1,1′- biphenyl]-3-carboxamide; 1-(3-Phenoxypropyl)-L- tryptophanol and 3′-Fluoro-4′-methoxy[1,1′- biphenyl]-3-carboxylic acid | 39 | (DMSO-d₆): 8.27 d (J=8.1 | |
| # Hz, 1H); 8.03 s (1H); 7.78 d (J=7.7 Hz, 1H); 7.77 d (J=7.5 Hz, 1H); 7.68 d (J=8.0 Hz, 1H); 7.64 dd (J=13.0 Hz/2.3 Hz, 1H); 7.52 d (J=8.9 Hz, 1H); 7.49 dd (J=7.7 Hz/7.5 Hz, 1H); 7.38 d (J=8.0 Hz, 1H); 7.27 dd (J=8.9 Hz/8.9 Hz, 1H); 7.24 dd (J=7.6 Hz/7.6 Hz, 2H); 7.19 s (1H); 7.06 dd (J=8.0 | | | | |
| # Hz/7.0 Hz, 1H); 6.98 dd (J=8.0 Hz/7.0 Hz, 1H); 6.90 dd (J=7.6 Hz/7.6 Hz, 1H); 6.84 d (J=7.6 Hz, 2H); 4.79 m (1H); 4.26 t (J=6.7 Hz, 2H); 4.25 m (1H); 3.88 s (3H); 3.54 m (1H); 3.51 m (1H); 3.02 dd (J=14.1 Hz/6.4 Hz, 1H); 3.80 t (J=6.0 Hz, 2H); 2.93 dd (J=14.1 Hz/7.7 Hz, 1H); 2.10 m (2H). | 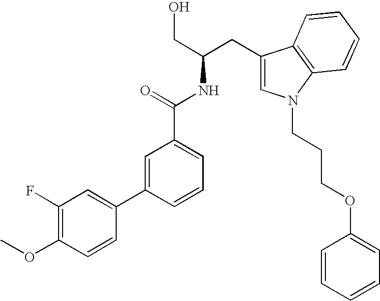 | | | |
| 405 | 3′-Fluoro-N-[(R)-1- (hydroxymethyl)-2-[1-(2- methoxyethyl)-1H-indol-3- yl]ethyl]-4′-methoxy[1,1′- biphenyl]-3-carboxamide; 1-(2-Methoxyethyl)-L- tryptophanol and 3′-Fluoro- 4′-methoxy[1,1′-biphenyl]- 3-carboxylic acid | 39 | (DMSO-d₆): 8.30 d (J=8.1 | |
| # Hz, 1H); 8.05 s (1H); 7.78 d (J=7.8 Hz, 1H); 7.78 d (J=7.6 Hz, 1H); 7.68 d (J=8.0 Hz, 1H); 7.65 dd (J=13.1 Hz/2.3 Hz, 1H); 7.53 d (J=8.8 Hz, 1H); 7.50 dd (J=7.8 Hz/7.6 Hz, 1H); 7.40 d (J=8.0 Hz, 1H); 7.28 dd (J=8.8 Hz/8.8 Hz, 1H); 7.18 s (1H); 7.09 dd (J=8.0 | | | | |
| # Hz/7.0 Hz, 1H); 6.99 dd (J=8.0 Hz/7.0 Hz, 1H); 4.82 m (1H); 4.25 m (1H); 4.23 t (J=5.3 Hz, 2H); 3.90 s (3H); 3.56 t (J=5.3 Hz, 2H); 3.55 m (1H); 3.51 m (1H); 3.10 s (3H); 3.04 dd (J=14.4 Hz/6.0 Hz, 1H); 2.93 dd (J=14.4 Hz/7.8 Hz, 1H). | 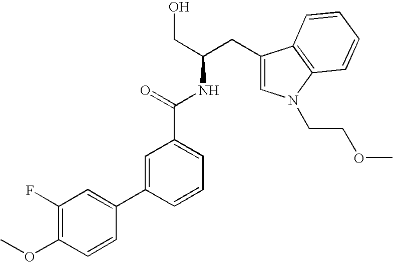 | | | |
| 406 | N-[(R)-2-[1-(3- Cyanopropyl)-1H-indol-3- yl]-1-(hydroxymethyl) ethyl]-3′-fluoro-4′- methoxy[1,1′-biphenyl]-3- carboxamide; 1-(3- Cyanopropyl)-L- tryptophanol and 3′-Fluoro- 4′-methoxy[1,1′- biphenyl]-3-carboxylic acid | 39 | (CDCl₃): 7.72 d (J=8.0 Hz, | |
| # 1H); 7.72 s (1H); 7.61 d (J=7.9 Hz, 1H); 7.59 d (J=7.5 Hz, 1H); 7.41 dd (J=7.9 Hz/7.5 Hz, 1H); 7.34 d (J=8.0 Hz, 1H); 7.03 s (1H); 7.29-7.19 m (3H); 7.13 dd (J=8.0 Hz/7.0 | | | | |
| # Hz, 1H); 7.01 dd (J=8.0 Hz/7.0 Hz, 1H); 6.58 d (J=7.4 Hz, 1H); 4.49 m (1H); 4.26 t (J=6.0 Hz, 2H); 3.94 s (3H); 3.83 m (1H); 3.79 m (1H); 3.17 m (2H); 2.18 m (2H); 2.16 m (2H). | 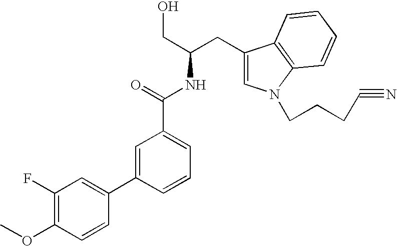 | | | |
| 407 | 4-Ethoxy-3′-methoxybi- phenyl-3-carboxylic acid [(R)-2-(1-cyanomethyl-1H- indol-3-yl)-1-hydroxy- methylethyl]amide; Methyl (R)-3-(1-cyanomethyl-1H- indol-3-yl)-2-[(4-ethoxy-3′- methoxybiphenyl-3- carbonyl)-amino]propionate | 335 | (DMSO-d₆): 8.42 d (J=7.8 Hz, 1H); 8.15 s (1H); 7.53 d (J=8.2 | |
| # Hz, 1H); 7.36 t (J=8.2 Hz, 1H); 7.25 s (1H); 7.20 m (3H); 7.13 m (3H); 6.91 d (9.3 Hz, 1H); 5.49 s (2H); 4.99 m (1H); 4.25 m (1H); 4.15 q (J=6.3 Hz, 2H); 3.82 s (3H); 3.50 m (1H); 3.46 m (1H); 3.00 m (2H); 1.31 t (J=6.6 Hz, 3H). | 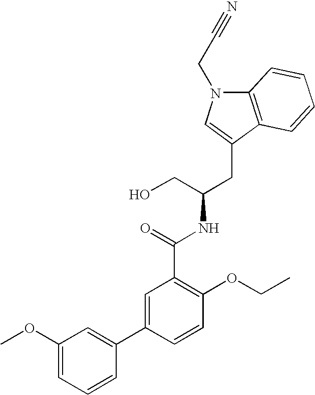 | | | |
| 408 | 6-Methoxy-2-(3,4,5-tri- methoxyphenyl)quinoline- 4-carboxylic acid [(R)-2-(1- cyanomethyl-1H-indol-3- yl)-1-hydroxymethylethyl]amide; Methyl (R)-3-(1- cyanomethyl-1H-indol-3- yl)-2-{[6-methoxy-2-(3,4,5- trimethoxyphenyl) quinoline-4-carbonyl]amino}propionate | 335 | (DMSO-d₆): 8.71 d (J=8.2 Hz, 1H); 8.00 t (J=4.3 Hz, 2H); | |
| # 7.73 d (J=7.9 Hz, 1H); 7.54 d (J=7.8 Hz, 1H); 7.50 s (2H); 7.46 s (1H); 7.41 m (1H); 7.30 s (1H); 7.21 t (J=7.6 Hz, 1H); 7.09 t (J=7.5 Hz, 1H); 5.50 s (2H); 4.95 t (J=5.5 Hz, 1H); 4.40 m (1H); 3.92 s (6H); 3.76 s (3H); 3.74 s (3H); 3.74 m (2H); 3.03 dd (J=14.4 Hz/5.5 Hz, 1H); 2.95 dd (J=14.4 Hz/8.1 Hz, 1H). | 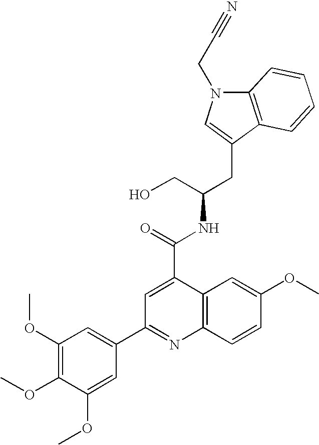 | | | |
| 409 | 6-Methoxy-2-(3,4,5-tri- methoxyphenyl)quinoline- 4-carboxylic acid {(R)-2- [1-(4-cyano-butyl)-1H- indol-3-yl]-1-hydroxy- methylethyl}amide; Methyl (R)-3-[1-(4- cyanobutyl)-1H-indol-3-yl]- 2-{[6-methoxy-2-(3,4,5- trimethoxyphenyl) quinoline-4-carbonyl]amino)propionate | 335 | (DMSO-d₆): 8.67 d (J=8.6 Hz, 1H); 8.01 d (J=7.8 | |
| # Hz, 1H); 8.00 s (1H); 7.68 d (J=7.8 Hz, 1H); 7.51 s (2H); 7.48 s (1H); 7.43 m (2H); 7.24 s (1H); 7.11 t (J=7.8 Hz, 1H); 6.98 t (J=7.4 Hz, 1H); 4.90 m (1H); 4.38 m (1H); 4.14 m (2H); 3.92 s (6H); 3.75 s (6H); 3.60 m (2H); 2.99 m (2H), 2.40 t (J=7.0, Hz, 2H); 1.76 t (J=7.5 Hz, 2H); 1.45 t (J=7.4 Hz, 2H). | 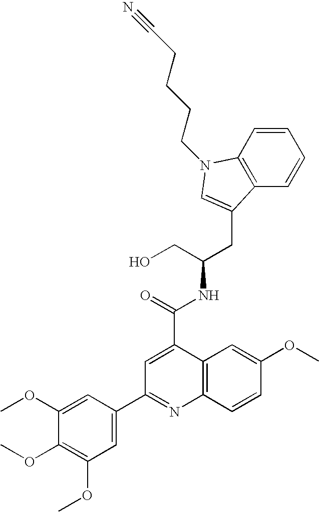 | | | |
| 410 | 4-Hydroxy-3′,4′,5′-tri- methoxybiphenyl-3- carboxylic acid [(R)-1- hydroxymethyl-2-(1H- indol-3-yl)ethyl]amide; (D)-Tryptophanol and 4-Hydroxy-3′,4′,5′- trimethoxybiphenyl-3- carboxylic acid | 39 | (DMSO-d₆): 12.49 s (1H); 10.76 s (1H); 8.67 d (J=8.1 Hz, 1H); | |
| # 8.10 d (J=2.1 Hz, 1H); 7.65 m (1H); 7.28 d (J=7.9 Hz, 1H); 7.12 s (1H); 6.93 m (3H); 6.84 s (2H); 4.88 m (1H); 4.26 m (1H); 3.84 s (6H); 3.65 s (3H); 3.50 m (2H); 2.97 m (2H). | 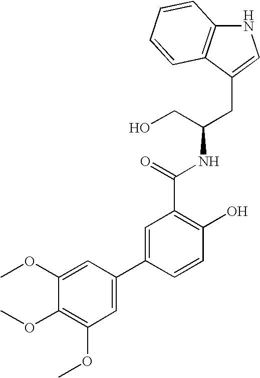 | | | |
| 411 | 4-(3-Cyanopropoxy)- 3′,4′,5′-trimethoxybiphenyl- 3-carboxylic acid [(R)-1- hydroxymethyl-2-(1H- indol-3-yl)ethyl]amide; 4-(3-Cyanopropoxy)- 3′,4′,5′-trimethoxybiphenyl- 3-carboxylic acid and (D)-Tryptophanol | 39 | (DMSO-d₆): 10.78 s (1H); 8.21 d (J=8.1 Hz, 1H); 8.02 d (J=2.6 Hz, 1H); 7.71 dd (J=2.5 Hz/8.5 | |
| # Hz, 1H); 7.68 d (J=7.7 Hz, 1H); 7.30 d (J=7.9 Hz, 1H); 7.19 d (J=8.7 Hz, 1H); 7.14 s (1H); 7.03 m (1H); 6.94 m (1H); 6.82 s (2H); 4.94 m (1H); 4.16 m (1H); 4.13 m (2H); 3.83 s (6H); 3.66 s (3H); 3.48 m (2H); 2.94 m (2H); 2.60 m (2H); 1.99 m (2H). | 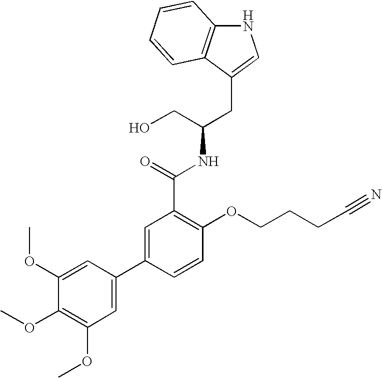 | | | |
| 412 | 4-Cyclopentyloxy-3′-fluoro- 4′-methoxybiphenyl-3- carboxylic acid [(R)-2- hydroxy-1-(1H-indol-3- ylmethyl)ethyl]amide; (D)-Tryptophanol and 4-Cyclopentyloxy-3′-fluoro- 4′-methoxybiphenyl-3- carboxylic acid | 39 | (DMSO-d₆): 10.81 s (1H); 8.36 d (J=8.2 Hz, 1H); 8.16 (1H); 7.70-7.74 m (2H); 7.51 d (J=12.9 | |
| # Hz, 1H); 7.42 d (J=8.6 Hz, 1H); 7.33 d (J=7.8 Hz, 1H); 7.23 t (J=8.8 Hz, 1H); 7.18 d (J=8.9 Hz, 1H); 7.15 s (1H); 7.06 t (J=7.4 Hz, 1H); 7.15 s (1H); 6.97 t (J=7.4 Hz, 1H); 5.01 m (1H); 4.97 t (J=4.9 Hz, 1H); 4.24-4.31 m (1H); 3.87 s (3H); 3.42-3.54 m (2H); 2.93-3.04 m (2H); 1.78-1.96 m (3H); 1.52-1.70 m (5H). | 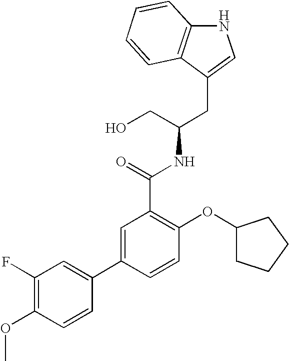 | | | |
| 413 | 4-Cyclopentyloxy-3′- methylbiphenyl-3- carboxylic acid [(R)-1- hydroxymethyl-2-(1H- indol-3-yl)ethyl]amide; (D)-Tryptophanol and 4-Cyclopentyloxy-3′- methylbiphenyl-3- carboxylic acid | 39 | (DMSO-d₆): 10.81 s (1H); 8.36 d (J=8.2 Hz, 1H); 8.21 (1H); 7.70-7.75 m (2H); 7.40-7.45 m (2H); 7.31-7.35 m (2H); 7.20 d | |
| # (J=8.6 Hz, 1H); 7.15 m (2H); 7.06 t (J=7.3 Hz, 1H); 6.97 t (J=7.4 Hz, 1H); 5.02 m (1H); 4.97 t (J=4.9 Hz, 1H); 4.24-4.31 m (1H); 3.42-3.54 m (2H); 2.94-3.04 m (2H); 2.38 s (3H); 1.79-1.94 m (3H); 1.64-1.73 m (3H); 1.51-1.61 m (2H). | 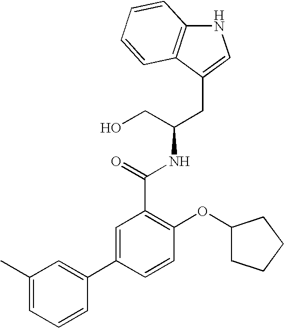 | | | |
| 414 | 3′-(1-Butyl-3-methyl- ureido)-4-cyclopentyloxy- biphenyl-3-carboxylic acid [(R)-1-hydroxy-methyl-2- (1H-indol-3-yl)ethyl]amide; (D)-Tryptophanol and 3′-(1-Butyl-3-methyl- ureido)-4-cyclopentyloxy- biphenyl-3-carboxylic acid | 39 | (DMSO-d₆): 10.81 s (1H); | |
| # 8.37 d (J=8.2 Hz, 1H); 8.22 d (J=2.3 Hz, 1H); 7.76 dd (J=8.6 Hz, J=2.3 Hz, 1H); 7.70 d (J=7.8 Hz, 1H); 7.53-7.50 m (1H); 7.48 t (J=7.8 Hz, 1H); 7.43 (1H); 7.31 d (J=8.2 Hz, 1H); 7.22 d (J=8.6 Hz, 1H); 7.14-7.18 m (2H); 7.05 t (J=7.4 Hz, 1H); | | | | |
| # 6.96 t (J=7.4 Hz, 1H); 5.66 q (J=4.3 Hz, 1H); 5.03 m (1H); 4.97 t (J=5.1 Hz, 1H); 4.24-4.32 m (1H); 3.61 t (J=7.4 Hz, 1H); 3.42-3.54 m (2H); 2.94-3.04 m (2H); 2.54 d (J=4.3 Hz, 3H); 1.79-1.95 m (3H); 1.52-1.73 m (5H); 1.36-1.43 m (2H); 1.21-1.29 m (2H) 0.84 t (J=7.4 Hz, 3H). | 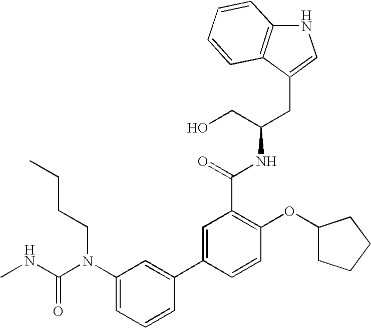 | | | |
| 415 | 4-Cyclopentyloxy-4′-fluoro- 3′-methylbiphenyl-3- carboxylic acid [(R)-2- hydroxy-1-(1H-indol-3- ylmethyl)ethyl]amide; (D)- Tryptophanol and 4-Cyclo- pentyloxy-4′-fluoro-3′- methylbiphenyl-3- carboxylic acid | 39 | (DMSO-d₆): 10.81 s | |
| # (1H); 8.37 d (J=8.2 Hz, 1H); 8.18 (1H); 7.72 s (1H); 7.70 s (1H); 7.55 d (J=7.4 Hz, 1H); 7.44-7.48 m (1H); 7.33 d (J=8.2 Hz, 1H); 7.20 t (J=7 | | | | |
| # Hz, 1H); 7.15 s (1H); 7.06 t (J=7.4 Hz, 1H); 6.97 t (J=7.4 Hz, 1H); 5.02 m (1H); 4.97 t (J=5.2 Hz, 1H); 4.25-4.32 m (1H); 3.42-3.54 m (2H); 2.94-3.04 m (2H); 2.31 s (3H); 1.79-1.96 m (3H); 1.51-1.73 m (5H). | 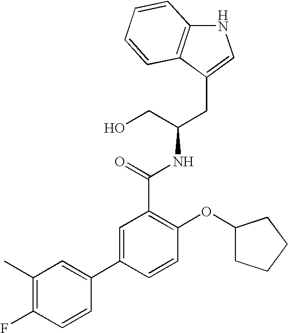 | | | |
| 416 | 4-Cyclopentyloxy-3′- methoxybiphenyl-3- carboxylic acid [(R)-1- hydroxymethyl-2-(1H- indol-3-yl)ethyl]amide; (D)-Tryptophanol and 4-Cyclopentyloxy-3′- methoxybiphenyl-3- carboxylic acid | 39 | (DMSO-d₆): 10.80 s (1H); | |
| # 8.37 d (J=8.2 Hz, 1H); 8.19 d (J=2.3 Hz, 1H); 7.76 dd (J=8.6 Hz, J=2.3 Hz, 1H); 7.70 d (J=7.8 Hz, 1H); 7.36 t (J=8 Hz, 1H); 7.33 d (J=7.8 Hz, 1H); 7.18-7.21 m (2H); 7.14 s (2H); 7.05 t (J=7.4 Hz, 1H); | | | | |
| # 6.96 t (J=7.2 Hz, 1H); 6.92 dd (J=8 Hz, J=2 Hz 1H); 5.03 m (1H); 4.96 t (J=5.1 Hz, 1H); 4.24-4.31 m (1H); 3.82 s (3H); 3.42-3.53 m (2H); 2.93-3.04 m (2H); 1.79-1.95 m (3H); 1.52-1.72 m (5H). | 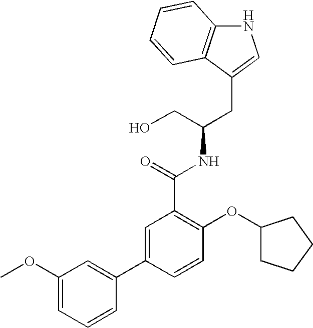 | | | |
| 417 | 4-Cyclopentyloxy-3′,4′- dimethoxybiphenyl-3- carboxylic acid [(R)-1- hydroxymethyl-2-(1H- indol-3-yl)ethyl]amide; (D)-Tryptophanol and 4-Cyclopentyloxy-3′,4′- dimethoxybiphenyl-3- carboxylic acid | 39 | (DMSO-d₆): 10.81 s (1H); 8.37 d (J=7.8 Hz, 1H); 8.16 d (J=2.3 | |
| # Hz, 1H); 7.69-7.73 m (2H); 7.36 d (J=8.2 Hz, 1H); 7.33 d (J=7.8 Hz, 1H); 7.14-7.19 m (4H); 7.03 t (J=8.2 Hz 1H); 6.96 t (J=7.42 Hz, 1H); 5.02 m (1H); 4.96 t (J=5 Hz, 1H); 4.24-4.31 m (1H); 3.84 s (3H); 3.79 s (3H); 3.41-3.53 m (2H); 2.93-3.04 m (2H); 1.79-1.93 m (3H); 1.52-1.72 m (5H). | 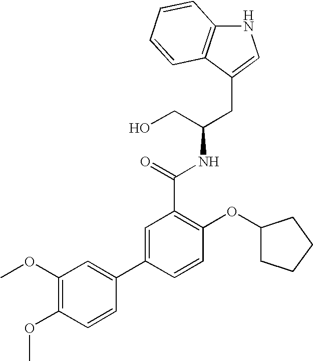 | | | |
| 418 | 5-Benzo[1,3]dioxol-5-yl-2- cyclopentyloxy-N-[(R)-1- hydroxymethyl-2-(1H- indol-3-yl)ethyl]benzamide; (D)-Tryptophanol and 5-Benzo[1,3]dioxol-5-yl-2- cyclopentyloxybenzoic acid | 39 | (DMSO-d₆): | |
| # 10.80 s (1H); 8.35 d (J=7.8 Hz, 1H); 8.11 d (J=1.9 Hz, 1H); 7.66-7.71 m (2H); 7.34 d (J=8.2 Hz, 1H); 7.14-7.19 m (3H); 7.04-7.10 m (2H); 6.94-6.99 m (2H); 6.05 s (2H); 5.0 m | | | | |
| # (1H); 4.95 t (J=5 Hz, 1H); 4.24-4.31 m (1H); 3.42-3.53 m (2H); 2.93-3.04 m (2H); 1.77-1.93 m (3H); 1.54-1.69 m (5H). | 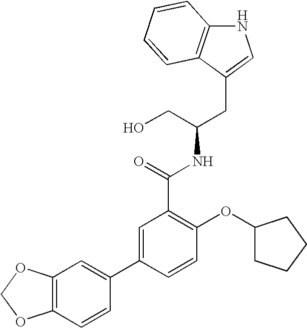 | | | |
| 419 | 4-Cyclopentyloxy-3′,4′,5′- trimethoxybiphenyl-3- carboxylic acid [(R)-1- hydroxymethyl-2-(1H- indol-3-yl)ethyl]amide; (D)-Tryptophanol and 4-Cyclopentyloxy-3′,4′,5′- trimethoxybiphenyl-3- carboxylic acid | 39 | (DMSO-d₆): 10.81 s (1H); | |
| # 8.37 d (J=7.8 Hz, 1H); 8.16 d (J=2.3 Hz, 1H); 7.76 dd (J=9 Hz, J=2.7 Hz, 1H); 7.70 d (J=7.8 Hz, 1H); 7.33 d (J=8.2 Hz, 1H); 7.19 d (J=8.9 Hz, 1H); 7.14 d (J=1.9 Hz, 1H); 7.05 t (J=7.4 Hz, 1H); 6.96 t | | | | |
| # (J=7.4 Hz, 1H); 6.85 s (2H); 5.04 m (1H); 4.97 t (J=5 Hz, 1H); 4.24-4.31 m (1H); 3.86 s (6H); 3.69 s (3H); 3.41-3.53 m (2H); 2.93-3.04 m (2H); 1.79-1.95 m (3H); 1.51-1.73 m (5H). | 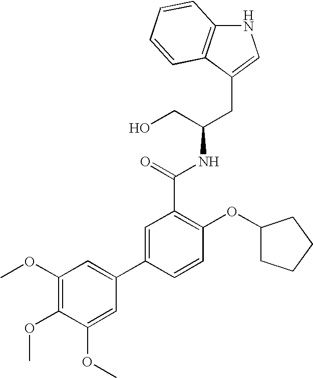 | | | |
| 420 | 4-Cyclopentyloxy-3′,4′- difluoro-5′-methoxy- biphenyl-3-carboxylic acid [(R)-2-hydroxy-1-(1H- indol-3-ylmethyl)ethyl]amide; (D)-Tryptophanol and 4-Cyclopentyloxy-3′,4′- difluoro-5′-methoxy- biphenyl-3-carboxylic acid | 39 | (DMSO-d₆): | |
| # 10.81 s (1H); 8.37 d (J=8.2 Hz, 1H); 8.19 d (J=2.3 Hz, 1H); 7.78 dd (J=8.7 Hz, J=2.6 Hz, 1H); 7.71 d (J=7.8 Hz, 1H); | | | | |
| # 7.33 d (J=7.8 Hz, 1H); 7.20-7.26 m (3H); 7.15 (1H); 7.06 t (J=7.4 Hz, 1H); 6.96 t (J=7.4 Hz, 1H); 5.04 m (1H); 4.97 t (J=5.1 Hz, 1H); 4.24-4.32 m (1H); 3.98 s (3H); 3.42-3.55 m (2H); 2.94-3.04 m (2H); 1.79-1.95 m (3H); 1.52-1.74 m (5H). | 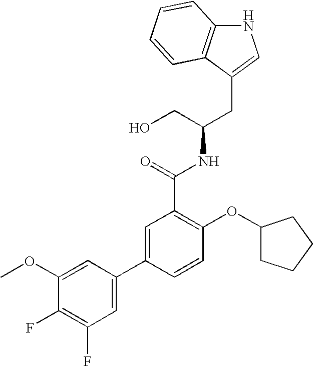 | | | |

### EXAMPLE 421

### 3′-[Butyl[(1,1-dimethylethoxy)carbonyl]amino]-4-ethoxy-N-[(R)-1-(hydroxymethyl)-2-(1H-indol-3-yl)ethyl][1,1′-biphenyl]-3-carboxamide

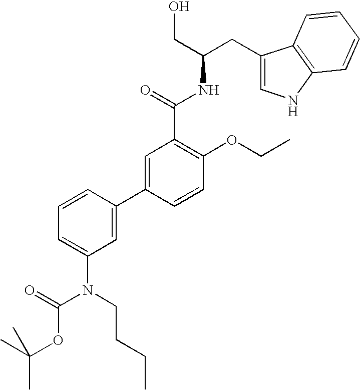

### 421a) tert-Butyl (3-bromophenyl)-n-butylcarbamate

tert-Butyl (3-bromophenyl)carbamate (56 g) were dissolved in DMF (250 ml), and NaH (60%, 10 g) was added in portions. The mixture was stirred until gas evolution was no longer observable and then 1-bromobutane (35 g) was slowly added dropwise. The mixture was stirred at 80° C. for two hours, cooled and poured into water (1000 ml). It was extracted with ethyl acetate (150 ml), and the organic phases were washed with water (3×100 ml), concentrated in a rotary evaporator and dried by azeotropic distillation with toluene. The title compound was obtained in quantitative yield (68 g). MS (ESI,+): 329 (M+1).

### 421b) 3-(tert-Butoxycarbonylbutylamino)phenylboronic acid

Butyllithium (1.6 M in hexane, 70 ml) was added dropwise to a solution of tert-butyl (3-bromophenyl)-n-butylcarbamate (31.4 g) in THF (400 ml) at −80° C. and, after stirring for 30 minutes, trimethyl borate (21.5 ml) was added dropwise. The reaction was thawed to room temperature, diluted with water (300 ml) and extracted with ethyl acetate, and the organic phases were dried over sodium sulphate. The residue was digested with hexane (200 ml) and water (20 ml) and stored in a refrigerator overnight. The product was filtered off and washed with cold hexane. Yield of the title compound 54% (16 g). MS (ESI,+): 294 (M+1).

### 407c) 3′-[Butyl[(1,1-dimethylethoxy)carbonyl]amino]-4-ethoxy-N-[(R)-1-(hydroxymethyl)-2-(1H-indol-3-yl)ethyl][1,1′-biphenyl]-3-carboxamide

The title compound was obtained in a Suzuki reaction in analogy to general method 125e.

(CD₃OD): 8.27 d (J=2.5 Hz, 1H); 7.72 dd (J=8.7 Hz/2.5 Hz, 1H); 7.66 d (J=8.0 Hz, 1H); 7.50 d (J=7.9 Hz, 1H); 7.45 m (1H); 7.33 d (J=8.0 Hz, 1H); 7.44 dd (J=7.9 Hz/7.7 Hz, 1H); 7.17 d (J=7.7 Hz, 1H); 7.15 d (J=8.7 Hz, 1H); 7.14s (1H); 7.07 dd (J=8.0 Hz/7.0 Hz, 1H); 6.95 dd (J=8.0 Hz/7.0 Hz, 1H); 4.49 m (1H); 4.11 m (1H); 4.03 m (1H); 3.69 m (2H); 3.67 m (2H); 3.14 m (2H); 1.53 m (2H); 1.45 s (9H); 1.35 m (2H); 1.24 t (J=7.2 Hz, 3H); 0.92 t (J=7.4 Hz, 3H).

### EXAMPLE 422

### 3′-(Butylamino)-4-ethoxy-N-[(R)-1-(hydroxymethyl)-2-(1H-indol-3-yl)ethyl][1,1′-biphenyl]-3-carboxamide;

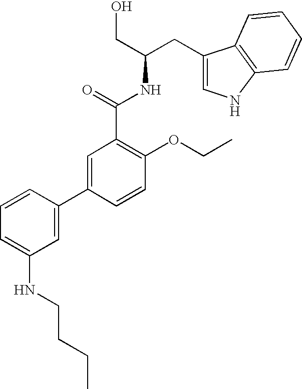

### 422a) 3-Butylaminophenylboronic acid

Ethereal HCl (saturated, 6 ml) was added to a solution of tert-butyl (3-bromophenyl)-n-butylcarbamate (500 mg) in dichloromethane (5 ml) and stirred at room temperature for six hours. The precipitate was filtered off, washed with diethyl ether, taken up in water (5 ml) and mixed with aqueous sodium bicarbonate solution. The precipitate was filtered off and washed with water. The title compound was obtained in 90% (350 mg) yield.

### 422b) 3′-(Butylamino)-4-ethoxy-N-[(R)-1-(hydroxymethyl)-2-(1H-indol-3-yl)ethyl][1,1′-biphenyl]-3-carboxamide

The title compound was obtained in a Suzuki reaction in analogy to general method 125e.

(CDCl₃): 8.50 d (J=7.3 Hz, 1H); 8.48 d (J=2.5 Hz, 1H); 8.13 s (1H); 7.71 d (J=8.0 Hz, 1H); 7.63 dd (J=8.6 Hz/2.5 Hz, 1H); 7.36 d (J=8.0 Hz, 1H); 7.22 dd (J=7.8 Hz/7.8 Hz, 1H); 7.19 dd (J=8.0 Hz/7.0 Hz, 1H); 7.11 dd (J=8.0 Hz/7.0 Hz, 1H); 7.10 s (1H); 6.95 d (J=8.6 Hz, 1H); 6.93 d (J=7.8 Hz, 1H); 6.86 m (1H); 6.60 d (J=7.8 Hz, 1H); 4.58 m (1H); 4.05 m (2H); 3.84 dd (J=10.9 Hz/3.5 Hz, 1H); 3.77 dd (J=10.9 Hz/5.3 Hz, 1H); 3.17 m (2H); 3.15 m (2H); 1.63 m (2H); 1.46 m (2H); 1.25 t (J=6.9 Hz, 3H); 0.97 t (J=7.3 Hz, 3H).

### EXAMPLE 423

### 3′-[Butyl[(methylamino)carbonyl]amino]-4-ethoxy-N-[(R)-1-(hydroxymethyl)-2-(1H-indol-3-yl)ethyl][1,1′-biphenyl]-3-carboxamide;

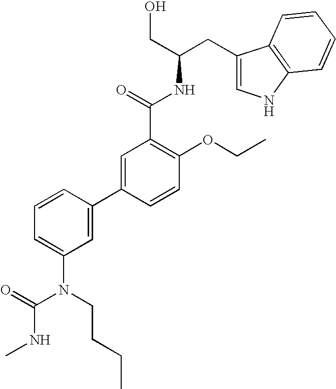

### 423a) 3-(1-Butyl-3-methylureido)phenylboronic acid

A solution of 3-butylaminophenylboronic acid (350 mg) and methyl isocyanate (103 mg) in THF (5 ml) was stirred at room temperature for one hour, a further 0.05 ml of methyl isocyanate was added, and the mixture was stirred at room temperature for a further three hours. The solvent was distilled off in a rotary evaporator, and the residue was recrystallized from ethanol. The title compound was obtained in 33% yield (150 mg).

### 423b) 3′-[Butyl[(methylamino)carbonyl]amino]-4-ethoxy-N-[(R)-1-(hydroxymethyl)-2-(1H-indol-3-yl)ethyl][1,1′-biphenyl]-3-carboxamide

The title compound was obtained in a Suzuki reaction in analogy to general method 125e.

(CD₃OD): 8.28 d (J=2.5 Hz, 1H); 7.75 dd (J=8.7 Hz/2.5 Hz, 1H); 7.65 d (J=8.0 Hz, 1H); 7.61 d (J=7.9 Hz, 1H); 7.52 dd (J=7.9 Hz/7.7 Hz, 1H); 7.49 m (1H); 7.33 d (J=8.0 Hz, 1H); 7.20 d (J=7.7 Hz, 1H); 7.16 d (J=8.7 Hz, 1H); 7.14 s (1H); 7.07 dd (J=8.0 Hz/7.0 Hz, 1H); 6.95 dd (J=8.0 Hz/7.0 Hz, 1H); 4.49 m (1H); 4.11 m (1H); 4.02 m (1H); 3.69 m (2H); 3.67 m (2H); 3.15 m (2H); 2.67 m (3H); 1.51 m (2H); 1.34 m (2H); 1.25 t (J=7.0 Hz, 3H); 0.91 t (J=7.4 Hz, 3H).

The following compounds were obtained in analogy to the preparation methods described in detail:

| Method | |
|---|---|
| Product; | analogous |
| reagents | to |
| 3′-(Butyl[(1,1-dimethyleth- | 421 |
| oxy)carbonyl]amino]-N-[(R)-1-(hydroxymethyl)-2-(1H-indol-3- | |
| yl)ethyl]-4-propoxy[1,1′-biphenyl]-3-carboxamide; | |
| D-Tryptophanol and 3′-[Butyl[(1,1- | |
| dimethylethoxy)carbonyl]amino]-4-propoxy[1,1′-biphenyl]-3-carboxylic acid | |
| 3′-(1-Butyl-3-methylureido)-4- | 423 |
| methoxybiphenyl-3-carboxylic acid | |
| [(R)-1-hydroxymethyl-2-(1H-indol-3-yl)ethyl]amide; | |
| (D)-Tryptophanol and | |
| 3′-(1-Butyl-3-methylureido)-4- | 423 |
| methoxybiphenyl-3-carboxylic acid | |
| 3′-(1-Butyl-3-methylureido)-4-methoxy-5-methylbiphenyl-3- | |
| carboxylic acid [(R)-1-hydroxy-methyl-2-(1H-indol-3-yl)ethyl]amide; | |
| (D)-Tryptophanol and | |
| 3′-(1-Butyl-3-methylureido)-4-methoxy-5-methylbiphenyl-3-carboxylic acid | |
| 3′-(1-Butyl-3-methylureido)-4- | 423 |
| isopropoxybiphenyl-3-carboxylic acid [(R)-1-hydroxymethyl-2-(1H-indol-3-yl)ethyl]amide; | |
| (D)- Ti′yptophanol and | |
| 3′-(1-Butyl-3-methylureido)-4-isopropoxybiphenyl-3-carboxylic acid | |
| 3′-(2-Dimethylaminoethoxy)-4- | 329 |
| ethoxybiphenyl-3-carboxylic acid | |
| [(R)-1 -hydroxymethyl-2-(1H-indol-3-yl)ethyl]amide; | |
| 5-Bromo-2-et hoxy-N-[(R)-1-hydroxymethyl-2-(1H-indol-3-yl)ethyl]benzamide and | |
| 3-(2-Dimethylaminoethoxy)-(2H);phenylboronic acid pinacol ester | |
| 4′-Ethoxy-3′-[(R)-1-hydroxymethyl- | 135 |
| 2-(1H-indol-3-yl)ethylcarbamoyl]-biphenyl-3-carboxylic acid methyl ester | |
| 5-Bromo-2-ethoxy-N-[(R)-1-hydroxymethyl-2-(1H-indol-3-yl)ethyl]benzamide and | |
| 3-Methoxycarbonylphenylboronic acid | |
| 4-Ethoxy-[1,1′; 3′,1″]terphenyl-3- | 135 |
| carboxylic acid [1-hydroxymethyl-2-(1H-indol-3-yl)ethyl]amide | |
| 5-Bromo-2-ethoxy-N-[(R)-1-hydroxymethyl-2-(1H-indol-3-yl)ethyl]benzamide and | |
| Biphenyl-3-boronic acid | |
| 3′-Acetyl-4-ethoxybiphenyl-3- | 135 |
| carboxylic acid [(R)-1-hydroxy-methyl-2-(1H-indol-3-yl)ethyl]amide; | |
| 5-Bromo-2-ethoxy-N-[(R)-1-hydroxymethyl-2-(1H-indol-3-yl)ethyl]benzamide and | |
| 3-Acetylphenylboronic acid | 135 |
| 4-Ethoxy-3′-pyrrolidin-1-yl-biphenyl-3-carboxylic acid [(R)-1- | |
| hydroxymethyl-2-(1H-indol-3-yl)ethyl]amide; | |
| 5-Bromo-2-ethoxy-N-[(R)-1-hydroxymethyl-2-(1H-indol-3- | |
| yl)ethyl]benzamide and | |
| (3-Pyrolidine-1-ylphenyl)boronic acid | |
| 4′-Cyanomethyl-4-ethoxybiphenyl- | 135 |
| 3-carboxylic acid [(R)-1-hydroxymethyl-2-(1H-indol-3-yl)ethyl]amide; | |
| 5-Bromo-2-ethoxy-N-[(R)-1-hydroxymethyl-2-(1H-indol-3-yl)ethyl]benzamide and | |
| (4-Cyanomethylphenyl)boronic acid | |
| 4′-Dimethylamino-4-propoxy- | 135 |
| biphenyl-3-carboxylic acid [(R)-1-hydroxymethyl-2-(1H-indol-3-yl)ethyl]amide; | |
| N-[(R)-1-Hydroxymethyl-2-(1H-indol-3-yl)ethyl]-5-iodo-2-propoxy-benzamide and | |
| 4-(Dimethylamino)phenylboronic acid | |
| 4-Propoxybiphenyl-3,3′-dicarboxylic | 135 |
| acid 3′-diethylamide 3-{[(R)-1-hydroxymethyl-2-(1H-indol-3-yl)ethyl]amide}; | |
| N-[(R)-1-Hydroxymethyl-2-(1H-indol-3-yl)ethyl]-5-iodo-2-propoxy-benzamide and | |
| 3-(N,N-Diethylaminocarbonyl)-phenylboronic acid | |
| 4-Propoxybiphenyl-3,4′-dicarboxylic | 135 |
| acid 4′-diethylamide 3-{[(R)-1-hydroxymethyl-2-(1H-indol-3-yl)ethyl]amide}; | |
| N-[(R)-1-Hydroxymethyl-2-(1H-indol-3-yl)ethyl]-5-iodo-2-propoxy-benzamide and | |
| 4-(N,N-Diethylaminocarbonyl)-phenylboronic acid | |
| 3′-[(R)-1-Hydroxymethyl-2-(1H- | 135 |
| indol-3-yl)ethylcarbamoyl]-4′-propoxybiphenyl-4-carboxylic acid; | |
| N-[(R)-2-Hydroxy- 1-(1H-indol-3-ylmethyl)ethyl]-5-iodo-2-propoxy-benzamide and | |
| 4-Carboxyphenylboronic acid | |
| 4′-Acetyl-4-propoxybiphenyl-3- | 135 |
| carboxylic acid [(R)-1-hydroxy-methyl-2-(1H-indol-3-yl)ethyl]amide; | |
| N-[(R)-2-Hydroxy-1-(1H-indol-3-ylmethyl)ethyl]-5-iodo-2-propoxy-benzamide and | |
| 4-Acetylphenylboronic acid | |
| 4′-Ethanesulphonyl-4-propoxy- | 135 |
| biphenyl-3-carboxylic acid [(R)-2-hydroxy-1-(1H-indol-3-ylmethyl)ethyl]amide; | |
| N-[(R)-2-Hydroxy-1-(1H-indol-3-ylmethyl)ethyl]-5-iodo-2-propoxy-benzamide and | |
| 4-(Ethylsulphonyl)phenylboronic acid | |
| 3′-Cyanomethyl-4-propoxy- | 135 |
| biphenyl-3-carboxylic acid [(R)-1-hydroxymethyl-2-(1H-indol-3-yl)ethyl]amide; | |
| N-[(R)-2-Hydroxy-1-(1H-indol-3-ylmethyl)ethyl]-5-iodo-2-propoxy-benzamide and | |
| 3-(Cyanomethyl)phenylboronic acid | |
| 3′-Methanesulphonylamino-4- | 135 |
| propoxy-biphenyl-3-carboxylic acid | |
| [(R)-2-hydroxy-1-(1H-indol-3-ylmethyl)ethyl]amide; | |
| N-[(R)-2-Hydroxy- 1-(1H-indol-3-ylmethyl)ethyl]-5-iodo-2-propoxy-benzamide and | |
| 3-(Methylsulphonylamino)phenyl-boronic acid | |
| 3′-Cyclopropylmethoxy-4-propoxy- | 135 |
| biphenyl-3-carboxylic acid [(R)-1-hydroxymethyl-2-(1H-indol-3-yl)ethyl]amide; | |
| N-[(R)-2-Hydroxy-1-(1H-indol-3-ylmethyl)ethyl]-5-iodo-2-propoxy-benzamide and | |
| 3-(Cyclopropylmethoxy)phenyl-boronic acid | |
| 3′-Methanesulphonyl-4-propoxy- | 135 |
| biphenyl-3-carboxylic acid [(R)-2-hydroxy-1-(1H-indol-3-ylmethyl)ethyl]amide; | |
| N-[(R)-2-Hydroxy-1-(1H-indol-3-ylmethyl)ethyl]-5-iodo-2-propoxy-benzamide and | |
| 3-(Methylsuofonyl)phenylboronic acid | |
| 4-Propoxybiphenyl-3,3′-dicarboxylic | 135 |
| acid 3′-R2-dimethyl-aminoethyl)amide] 3-{[(R)-1- | |
| hydroxymethyl-2-(1H-indol-3-yl)ethyl]amide}; | |
| N-[(R)-2-Hydroxy-1-(1H-indol-3-ylmethyl)ethyl]-5-iodo-2-propoxy-benzamide and | |
| 3-(2-N,N-Dimethylaminoethylamino-carbonyl)phenylboronic acid | |
| 3′-[(R)-1-Hydroxymethyl-2-(1H- | 135 |
| indol-3-yl)ethylcarbamoyl]-3-methoxy-4′-propoxybiphenyl-4-carboxylic acid methyl ester; | |
| N-[(R)-2-Hydroxy-1-(1H-indol-3-ylmethyl)ethyl]-5-iodo-2-propoxy-benzamide and | |
| 3-Methoxy-4-(methoxycarbonyl)-phenylboronic acid | |
| 3′-Chloro-4-propoxybiphenyl-3,4′- | 135 |
| dicarboxylic acid 4′-amide 3-{[(R)-2-hydroxy-1-(1H-indol-3-ylmethyl)ethyl]amide}; | |
| N-[(R)-2-Hydroxy-1-(1H-indol-3-ylmethyl)ethyl]-5-iodo-2-propoxy-benzamide and | |
| 4-(Aminocarbonyl)-3-chlorophenyl-boronic acid | |
| 3′-Dimethylsulphamoyl-4-propoxy- | 135 |
| biphenyl-3-carboxylic acid [(R)-2-hydroxy-1-(1H-indol-3-ylmethyl)ethyl]amide; | |
| N-[(R)-2-Hydroxy-1-(1H-indol-3-ylmethyl)ethyl]-5-iodo-2-propoxy-benzamide and | |
| 3(N,N-Dimethylsulphonamidophenyl)-boronic acid | |
| 4′-(Propane-2-sulphonyl)-4- | 135 |
| propoxy-biphenyl-3-carboxylic acid | |
| [(R)-2-hydroxy-1-(1H-indol-3-ylmethyl)ethyl]amide; | |
| N-[(R)-2-Hydroxy-1-(1H-indol-3-ylmethyl)ethyl]-5-iodo-2-propoxy-benzamide and | |
| 4-(Isopropylsulphonylphenyl)-boronic acid | |
| 4′-Methylsulphamoyl-4-propoxy- | 135 |
| biphenyl-3-carboxylic acid [(R)-2-hydroxy-1-(1H-indol-3-ylmethyl)ethyl]amide; | |
| N-[(R)-2-Hydroxy-1-(1H-indol-3-ylmethyl)ethyl]-5-iodo-2-propoxy-benzamide and | |
| (4-Methylaminosulphonylphenyl)-boronic acid | |
| 4′-Dimethylsulphamoyl-4-propoxy- | 135 |
| biphenyl-3-carboxylic acid [(R)-2-hydroxy-1-(1H-indol-3-ylmethyl)ethyl]amide; | |
| N-[(R)-2-Hydroxy-1-(1H-indol-3-ylmethyl)ethyl]-5-iodo-2-propoxy-benzamide and | |
| (4-Dimethylaminosulphonylphenyl)-boronic acid | |
| 4-Propoxybiphenyl-3,4′-dicarboxylic | 135 |
| acid 4′-amide 3-{[(R)-1-hydroxymethyl-2-(1H-indol-3-yl)ethyl]amide}; | |
| N-[(R)-2-Hydroxy-1-(1H-indol-3-ylmethyl)ethyl]-5-iodo-2-propoxy-benzamide and | |
| 4-Aminocarbonylphenylboronic acid | |
| 3′-Methylsulphamoyl-4-propoxy- | 135 |
| biphenyl-3-carboxylic acid [(R)-2-hydroxy-1-(1H-indol-3-ylmethyl)ethyl]amide; | |
| N-[(R)-2-Hydroxy-1-(1H-indol-3- ylmethyl)ethyl]-5-iodo-2-propoxy- benzamide and | |
| (3-Methylaminosulphonylphenyl)- boronic acid | |
| 3′-Methanesulphonyl-4-propoxy- | 135 |
| biphenyl-3-carboxylic acid [(R)-2-hydroxy-1-(1-methyl-1H-indol-3-ylmethyl)ethyl]amide; | |
| N-[(R)-1-Hydroxymethyl-2-(1-methyl-1H-indol-3-yl)ethyl]-5-iodo-2-propoxybenzamide and | |
| 3-Methylsulphonylphenylboronic acid | |
| 3′-[(R)-1-Hydroxymethyl-2-(1- | 135 |
| methyl-1H-indol-3-yl)ethylcarbamoyl]-3-methoxy-4′-propoxybiphenyl-4-carboxylic acid-methyl ester; | |
| N-[(R)-1-Hydroxymethyl-2-(1-methyl-1H-indol-3-yl)ethyl]-5-iodo-2-propoxybenzamide and | |
| 3-Methoxy-4-(methoxycarbonyl)-phenylboronic acid | |
| 4-Propoxybiphenyl-3,4′-dicarboxylic | 135 |
| acid 4′-[(2-dimethylaminoethyl)amide] 3-{[(R)-1-hydroxymethyl-2-(1-methyl-1H- | |
| indol-3-yl)ethyl]amide}; | |
| N-[(R)-1-Hydroxymethyl-2-(1-methyl-1H-indol-3-yl)ethyl]-5-iodo-2-propoxybenzamide and | |
| 3-(2-N,N-Dimethylaminoethyl-aminocarbonyl)phenylboronic acid | |
| 3′-[2-(5-Fluoro-1H-indol-3-yl)-1- | 135 |
| hydroxymethylethylcarbamoyl]-4′-propoxybiphenyl-4-carboxylic acid; | |
| N-[2-(5-Fluoro-1H-indol-3-yl)-1-hydroxymethyl-ethyl]-5-iodo-2-propoxybenzamide and | |
| 4-Carboxyphenylboronic acid | |
| 3′-Methanesulphonylamino-4- | 135 |
| propoxy-biphenyl-3-carboxylic acid | |
| [2-(5-fluoro-1H-indol-3-yl)-1-hydroxymethylethyl]amide; | |
| N-[2-(5-Fluoro-1H-indol-3-yl)-1-hydroxymethylethyl]-5-iodo-2-propoxybenzamide and | |
| 3-(Methylsulphonylamino)phenyl-boronic acid | |
| 3′-Methanesulphonyl-4-propoxy- | 135 |
| biphenyl-3-carboxylic acid [2-(5-fluoro-1H-indol-3-yl)-1- | |
| N-[2-(5-Fluoro-1H-indol-3-yl)-1-hydroxymethylethyl]amide; | |
| hydroxymethylethyl]-5-iodo-2-propoxybenzamide and | |
| 3-Methylsulphonylphenylboronic acid | |
| 4-Propoxybiphenyl-3,3′-dicarboxylic | 135 |
| acid 3′-[(2- dimethylaminoethyl)amide] 3-{[2-(5- | |
| fluoro-1H-indol-3-yl)-1-hydroxymethylethyl]amidel; | |
| hydroxymethylethyl]amide}; | |
| N-[2-(5-Fluoro-1H-indol-3-yl)-1-hydroxymethylethyl]-5-iodo-2-propoxybenzamide and | |
| 3-(2-N,N-Dimethylaminoethyl-aminocarbonyl)phenylboronic acid | |
| 3′-Chloro-4-propoxybiphenyl-3,4′- | 135 |
| dicarboxylic acid 4′-amide 3-{[2-(5-fluoro-1H-indol-3-yl)-1-hydroxymethylethyl]amide}; | |
| N-[2-(5-Fluoro-1H-indol-3-yl)-1-hydroxymethylethyl]-5-iodo-2-propoxybenzamide and | |
| 4-(Aminocarbonyl)-3-chlorophenyl-boronic acid | |
| 3′-Dimethylsulphamoyl-4-propoxy- | 135 |
| biphenyl-3-carboxylic acid [2-(5-fluoro-1H-indol-3-yl)-1-hydroxymethylethyl]amide; | |
| N-[2-(5-Fluoro-1H-indol-3-yl)-1-hydroxymethylethyl]-5-iodo-2-propoxybenzamide and | |
| 3-(N,N-Dimethylsulphonamido-phenyl)boronic acid | |
| 4′-(Propane-2-sulphonyl)-4- | 135 |
| propoxy-biphenyl-3-carboxylic acid | |
| [2-(5-fluoro-1H-indol-3-yl)-1-hydroxymethylethyl]amide; | |
| N-[2-(5-Fluoro-1H-indol-3-yl)-1-hydroxymethylethyl]-5-iodo-2-propoxy-benzamide and | |
| 4-(Isopropylsulphonylphenyl)-boronic acid | |
| 4′-Dimethylsulphamoyl-4-propoxy- | 135 |
| biphenyl-3-carboxylic acid [2-(5-fluoro-1H-indol-3-yl)-1-hydroxymethylethyl]amide; | |
| N-[2-(5-Fluoro-1H-indol-3-yl)-1-hydroxymethylethyl]-5-iodo-2-propoxybenzamide and | |
| 4-(N,N-Dimethylsulphonamido-phenyl)boronic acid | |
| 4-Propoxybiphenyl-3,4′-dicarboxylic | 135 |
| acid 4-diethylamide 3-{[2-(5-fluoro-1H-indol-3-yl)-1-hydroxymethylethyl]amide}; | |
| N-[2-(5-Fluoro-1H-indol-3-yl)-1-hydroxymethylethyl]-5-iodo-2-propoxybenzamide and | |
| 4-(N,N-Dimethylaminocarbonyl)-phenylboronic acid | |
| 3′-Methylsulphamoyl-4-propoxy- | 135 |
| biphenyl-3-carboxylic acid [2-(5-fluoro-1H-indol-3-yl)-1-hydroxymethylethyl]amide; | |
| N-[2-(5-Fluoro-1H-indol-3-yl)-1-hydroxymethylethyl]-5-iodo-2-propoxybenzamide and | |
| (3-Methylaminosulphonylphenyl)-boronic acid | |
| 3′-Acetyl-4-propoxybiphenyl-3- | 135 |
| carboxylic acid [(R)-1-hydroxymethyl-2-(1-methyl-1H-indol-3-yl)ethyl]amide; | |
| N-[(R)-1-Hydroxymethyl-2-(1-methyl-1H-indol-3-yl)ethyl]-5-iodo-2-propoxybenzamide and | |
| 3-Acetylphenylboronic acid | |
| 4-Propoxy-[1.1′; 3′.1′]terphenyl-3- | 135 |
| carboxylic acid [(R)-1-hydroxymethyl-2-(1-methyl-1H-indol-3-yl)ethyl]amide; | |
| N-[(R)-1-Hydroxymethyl-2-(1-methyl-1H-indol-3-yl)ethyl]-5-iodo-2-propoxy-benzamide and | |
| Biphenyl-3-boronic acid | |
| 3′-Cyanomethyl-4-propoxy- | 135 |
| biphenyl-3-carboxylic acid [(R)-1-hydroxymethyl-2-(1-methyl-1H-indol-3-yl)ethyl]amide; | |
| N-[(R)-1-Hydroxymethyl-2-(1-methyl-1H-indol-3-yl)ethyl]-5-iodo-2-propoxybenzamide and | |
| 3-Cyanomethylphenylboronic acid | |
| 3′-Methansulphonylamino-4- | 135 |
| propoxy-biphenyl-3-carboxylic acid | |
| [(R)-2-hydroxy-1-(1-methyl-1H-indol-3-ylmethyl)ethyl]amide; | |
| N-[(R)-1-Hydroxymethyl-2-(1-methyl-1H-indol-3-yl)ethyl]-5-iodo-2-propoxybenzamide and | |
| 3-(Methylsulphonamido)phenyl-boronic acid | |
| 4′-Cyanomethyl-4-propoxy- | 135 |
| biphenyl-3-carboxylic acid [(R)-1-hydroxymethyl-2-(1-methyl-1H-indol-3-yl)ethyl]amide; | |
| N-[(R)-1-Hydroxymethyl-2-(1-methyl-1H-indol-3-yl)ethyl]-5-iodo-2-propoxybenzamide and | |
| 4-Cyanomethylphenylboronic acid | |
| 4-Propoxy-biphenyl-3.3′- | 135 |
| dicarboxylic acid 3′-[(2-dimethylamino-ethyl)amide] 3{[(R) | |
| 1-hydroxymethyl-2-(1-methyl-1H-indol-3-yl)ethyl]amide}; | |
| N-[(R)-1-Hydroxymethyl-2-(1-methyl-1H-indol-3-yl)ethyl]-5-iodo-2-propoxybenzamide and | |
| 3-(2-N,N-Dimethylaminoethyl-aminocarbonyl)phenylboronic acid | |
| 4-Fluoro-3′-[(R)-2-hydroxy-1-(1- | 135 |
| methyl-1H-indol-3-ylmethyl)ethylcarbamoyl]-4′-propoxy-biphenyl-3-carboxylic acid; | |
| N-[(R)-1-Hydroxymethyl-2-(1-methyl-1H-indol-3-yl)ethyl]-5-iodo-2-propoxy-benzamide and | |
| 3-Carboxy-4-fluorphenylboronic acid | |
| 3′-Chloro-4-propoxybiphenyl-3,4′- | 135 |
| dicarboxylic acid 4′-amide 3-{[(R)-2-hydroxy-1-(1-methyl-1H-indol-3-ylmethyl)ethyl]amide}; | |
| N-[(R)-1-Hydroxymethyl-2-(1-methyl-1H-indol-3-yl)ethyl]-5-iodo-2-propoxybenzamide and | |
| 3-Chloro-5-(carbamoyl)phenyl-boronic acid | |
| 4-Propoxybiphenyl-3,4′-dicarboxylic | 135 |
| acid 4-diethylamide 3-{[(R)-1-hydroxymethyl-2-(1-methyl-1H-indol-3-yl)ethyl]amide}; | |
| N-[(R)-1-Hydroxymethyl-2-(1-methyl-1H-indol-3-yl)ethyl]-5-iodo-2-propoxybenzamide and | |
| 4-(N,N-Diethylaminocarbonyl)-phenylboronic acid | |
| 4′-Dimethylamino-4-propoxy- | 135 |
| biphenyl-3-carboxylic acid[2-(5-fluoro-1H-indol-3-yl)-1-hydroxymethylethyl]amide; | |
| N-[2-(5-Fluoro-1H-indol-3-yl)-1-hydroxymethylethyl]-5-iodo-2-propox-benzamide and | |
| 4-Dimethylaminophenylboronic acid | |
| 4′-Acetyl-4-propoxybiphenyl-3- | 135 |
| carboxylic acid [2-(5-fluoro-1H-indol-3-yl)-1-hydroxymethylethyl]amide; | |
| N-[2-(5-Fluoro-1H-indol-3-yl)-1-hydroxymethylethyl]-5-iodo-2-propoxybenzamide and | |
| 4-Acetylphenylboronic acid | |
| 3′-Acetyl-4-propoxybiphenyl-3- | 135 |
| carboxylic acid [2-(5-fluoro-1H-indol-3-yl)-1-hydroxymethylethyl]amide; | |
| N-[2-(5-Fluoro-1H-indol-3-yl)-1-hydroxymethylethyl]-5-iodo-2-propoxybenzamide and | |
| 3-Acetylphenylboronic acid | |
| 4-Propoxy-[1.1′; 3′.1″]terphenyl-3- | 135 |
| carboxylic acid [2-(5-fluoro-1H-indol-3-yl)-1-hydroxymethylethyl]amide; | |
| N-[2-(5-Fluoro-1H-indol-3-yl)-1-hydroxymethylethy]-5-iodo-2-propoxy-benzamide and | |
| Biphenyl-3-boronic acid | |
| 3′-[2-(5-Fluoro-1H-indol-3-yl)-1- | 135 |
| hydroxymethylethylcarbamoyl]-3-methoxy-4′-propoxy-biphenyl-4-carboxylic acidmethyl ester; | |
| N-[2-(5-Fluoro-1H-indol-3-yl)-1-hydroxymethylethy]-5-iodo-2-propoxy-benzamide and | |
| 3-Methoxy-4-(methoxycarbonyl)-phenylboronic acid | |
| 4-Propoxybiphenyl-3,4′-dicarboxylic | 135 |
| acid 4-amide 3-{(2-(5-fluoro-1H-indol-3-yl)-1-hydroxymethylethyl]amide}; | |
| N-[2-(5-Fluoro-1H-indol-3-yl)-1-hydroxymethylethyl]-5-iodo-2-propoxybenzamide and | |
| 4-Aminocarbonylphenylboronic acid | |
| 4-Ethoxy-4′-methoxymethyl- | 125 |
| biphenyl-3-carboxylic acid [(R)-1-hydroxymethyl-2-(1H-indol-3-yl)ethyl]amide; | |
| 5-Bromo-2-ethoxy-N-[(R)-1-hydroxymethyl-2-(1H-indol-3-yl)ethyl]benzamide and | |
| 4-Methoxymethylphenylboronic acid | |
| 4-Ethoxybiphenyl-3,3′-dicarboxylic | 125 |
| acid 3′-amide 3-{[(R)-1-hydroxymethyl-2-(1H-indol-3-yl)ethyl]amide}; | |
| 5-Bromo-2-ethoxy-N-[(R)-1-hydroxymethyl-2-(1H-indol-3-yl)ethyl]benzamide and | |
| 3-Aminocarbonylphenylboronic acid | |
| 4-Ethanesulphonyl-4- | 125 |
| ethoxybiphenyl-3-carboxylic | |
| [(R)-2-hydroxy-1-(1H-indol-3-ylmethyl)ethyl]amide; | |
| 5-Bromo-2-ethoxy-N-[(R)-1-hydroxymethyl-2-(1H-indol-3-yl)ethyl]benzamide and | |
| 4-(Ethylsulphonyl)phenylboronic acid | |
| 4-Ethoxy-4′-(4-methylpiperazin-1- | 125 |
| carbonyl)biphenyl-3-carboxylic acid | |
| [(R)-1-hydroxymethyl-2-(1H-indol-3-yl)ethyl]amide; | |
| 5-Bromo-2-ethoxy-N-[(R)-1-hydroxymethyl-2-(1H-indol-3-yl)ethyl]benzamide and | |
| 4-(4-Methylpiperazin-1-carbonyl)-phenylboronic acid | |
| 3′-Cyclopropylmethoxy-4-ethoxy- | 125 |
| biphenyl-3-carboxylic acid [(R)-1-hydroxymethyl-2-(1H-indol-3-yl)ethyl]amide; | |
| 5-Bromo-2-ethoxy-N-[(R)-1-hydroxymethyl-2-(1H-indol-3-yl)ethyl]benzamide and | |
| 3-(Cyclopropylmethoxy)phenyl-boronic acid | |
| 3′-((R)-1-Hydroxymethyl-2-(1H- | 125 |
| indol-3-yl)ethylcarbamOyl]biphenyl-2-carboxylic acid methyl ester; | |
| 3-Bromo-N-[(R)-1-hydroxymethyl-2-(1H-indol-3-yl)ethyl]benzamide and | |
| (2-Methoxycarbonylphenyl)boronic acid | |

| ¹H-NMR(400 MHz) δ [ppm] | Structure |
|---|---|
| (CDCl₃): 8.48 δ(J=2.5 Hz, 1H); 8.44 δ(J=7.1Hz, 1H); 7.71 δ(J=8.0 Hz, 1H); 7.63 dd(J=8.7 Hz / 2.5 Hz, 1H); 7.44 δ(J=8.1Hz, 1H); 7.43 m(1H); 7.37 dd(J=8.1 Hz/ 8.1Hz, 1H); 7.36 δ(J=8.0 Hz, 1H); 7.19 dd(J=8.0 Hz/ 7.0 Hz, 1H); 7.14 m(1H); 7.11 dd(J=8.0 Hz/7.0 Hz, 1H); 7.10s(1H); 6.99d(J=8.7 Hz, 1H); 4.59 m(1H); 3.96 m (2H); 3.88 m(1H); 3.78 m (1H); 3.66 m(2H); 3.15 m | |
| # (2H); 1.64 m(2H); 1.54 m (2H); 1.45 s(9H); 1.32 m (2H); 0.94 t(J=7.4 Hz, 3H); 0.90 t(J=7.4 Hz, 3H). | 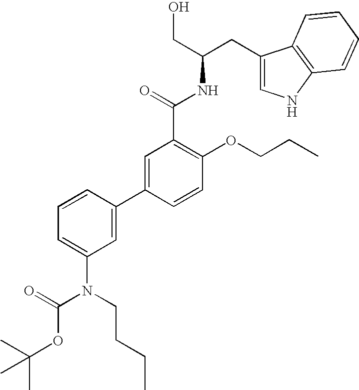 |
| (DMSO-d₆): 10.85 s(1H); 8.19 δ(J=7.8 Hz, 1H); 8.19 (1H); 7.78 δ(J=8.6 Hz, 1H); 7.69 δ(J=7.8 Hz, 1H);7.53d (J=7.8 Hz, 1H); 7.47 t(J=7.8 Hz, 1H); 7.41(1H); 7.33 d (J=8.2Hz, 1H); 7.22-7.15 m (3H); 7.06 t(J=7.4 Hz, 1H); 6.98 t(J=7.4 Hz, 1H); 5.65 q (J=4.3 Hz, 1H); 4.94(1H); 4.21-4.29 m(1H); 3.84 s(3H); 3.60 t(J=7.2 Hz, 2H); 3.41-3.57 m(2H); 2.95-3.06 m (2H); | |
| # 2.53 δ(J=4.3 Hz, 3H); 1.35-1.42 m(2H); 1.21-1.26m (2H); 0.83 t(J=7.4 Hz, 3H). | 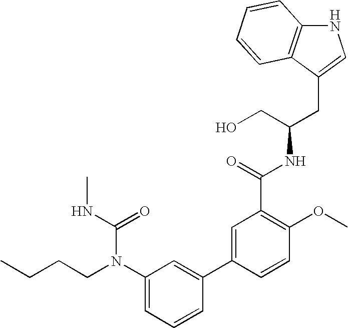 |
| (DMSO-d₆): 10.80 s(1H); 8.25 δ(J=8.2 Hz, 1H); 7.68 d (J=7.8 Hz, 1H); 7.63 m(2H); 7.46-7.52 m(2H); 7.43 s(1H); 7.32 δ(J=8.2 Hz, 1H); 7.18 m(2H); 7.06 t(J=7.4 Hz, 1H); 6.97 t(J=7.4 Hz, 1H); 5.65 q(J=4.3 Hz, 1H); 4.89t (J=5.5 Hz, 1H); 4.23-4.31 m (1H); 3.62 s(3H); 3.53-3.59 m (3H); 3.45-3.50 m(1H); 3.04 dd(J=14.4 Hz, J=6.6 Hz, 1H); | |
| # 2.94 dd(J=14.4 Hz, J=6.6 Hz, 1H); 2.54 δ(J=4.3 Hz, 3H); 2.31 s (3H); 1.35-1.42 m(2H); 1.21-1.28 m (2H); 0.83t(J=7.4 Hz, 3H). | 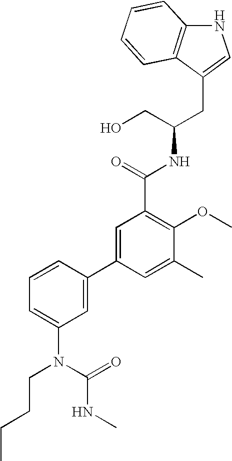 |
| (DMSO-d₆): 10.82 s(1H); 8.48 δ(J=8.2 Hz, 1H); 8.20d (J=2.3 Hz, 1H); 7.75 dd(J=8.2 Hz, J=2.1Hz, 1H); 7.71d(J=7.8 Hz, 1H);7.54d(J=7.8 Hz, 1H); 7.48t(J=7.8 Hz, 1H); 7.42(1H); 7.33 δ(J=8.2 Hz, 1H); 7.26 δ(J=8.9 Hz, 1H); 7.16-7.18 m(2H); 7.05t (J=7.4 Hz, 1H); 6.98t(J=7.2 Hz, 1H); 5.65 q(J=4.3 Hz, 1H); 4.97t(J=4.8 Hz, 1H); | |
| # 4.79-4.85 m(1H); 4.23-4.30 m(1H); 3.61t(J=7.4 Hz, 2H); 3.421-3.54 m(2H); 2.94-3.04 m(2H); 2.53 δ(J=4.3 Hz, 3H); 1.36-1.43 m(2H); 1.21-1.29 m(8H); 0.84t(J=7.4 Hz, 3H). | 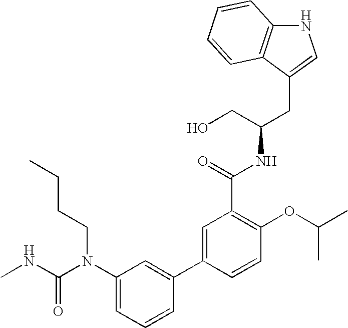 |
| (CDCl₃): 8.78s(1H); 8.50 δ(J=7.3 Hz, 1H); 8.46 δ(J=2.5 Hz, 1H); 7.71 δ(J=7.8 Hz, 1H); 7.56dd(J=2.5 Hz/8.6 Hz, 1H); 7.32m(2H); 7.13m (3H); 7.06m(2H); 6.89m (2H); 4.55m(1H); 4.11m (2H); 3.97m(2H); 3.74m (2H); 3.12m(2H); 2.78m 2.37 s(6H); 1.21m (3H). | 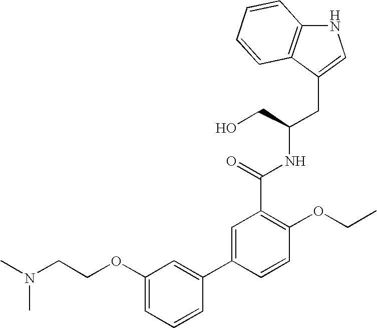 |
| Column Purospher Star RP C18 4.6 × 125 5 μm; detection wavelength 214 nm; flow rate 1 ml/min; eluents A: 0.1% TFA in. H₂O, B 0.1% TFA in ACN; gradient in each case based on B: 5% to 95%(10′) to 95% (2′) to 5%(0.5′) to 5%(2.5′) Molecular peak(ESI, M + 1): 473.5 Retention time: 9.95 min. | 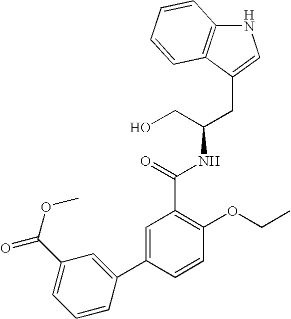 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 491.6 Retention time: 11.1 min. | 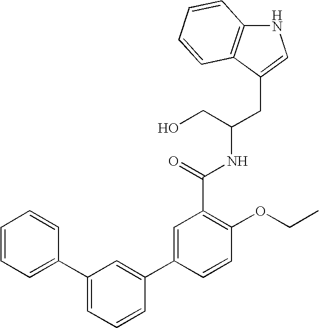 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 457.5 Retention time: 9.1 min. | 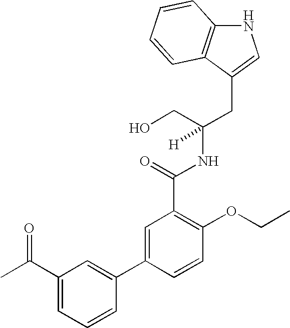 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 484.6 Retention time: 8.75 min. | 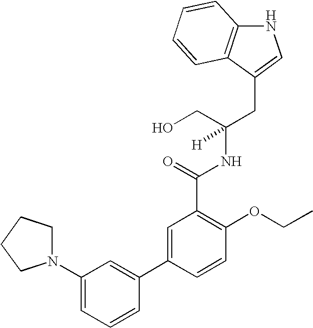 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 454.5 Retention time: 9.03 min. | 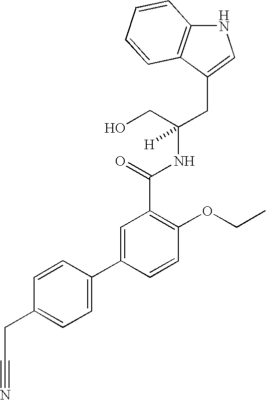 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% on B: 5% to 95%(10′) to 95% (2′) to 5%(0.5′) to 5%(2.5′) Molecular peak(ESI, M + 1): 472.5 Retention time: 6.95 min. | 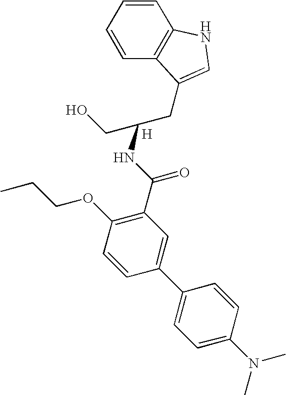 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 528.5 Retention time: 8.95 min. | 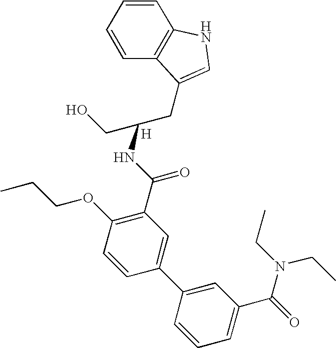 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 528.5 Retention time: 8.88 min. | 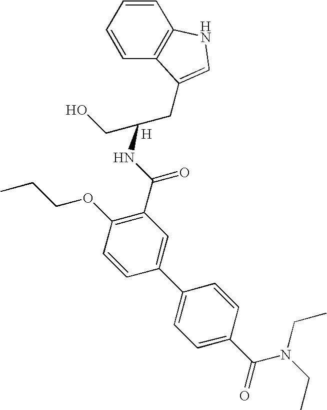 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 473.5 Retention time: 8.35 min. | 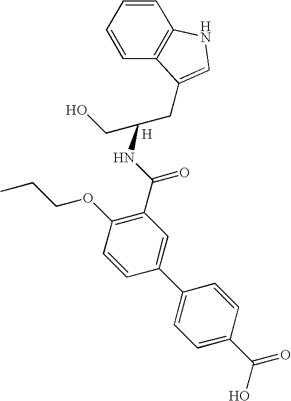 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 471.5 Retention time: 9.15 min. | 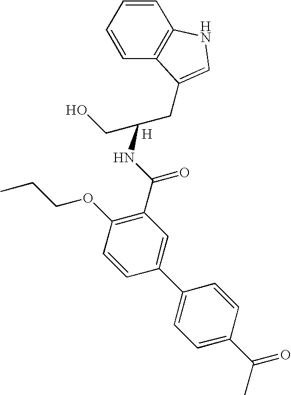 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% on B: 5% to 95%(10′) to 95% (2′) to 5%(0.5′) to 5%(2.5′) Molecular peak(ESI, M + 1): 521.5 Retention time: 8.73 min. | 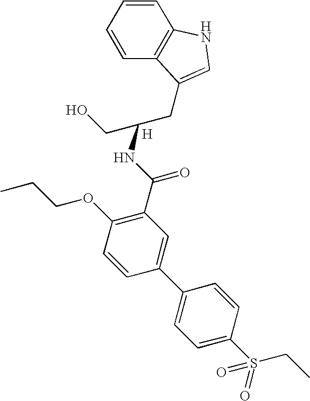 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 468.5 Retention time: 9.13 min. | 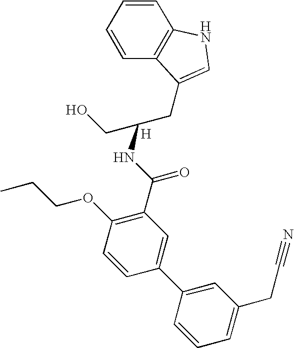 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 522.5 Retention time: 8.56 min. | 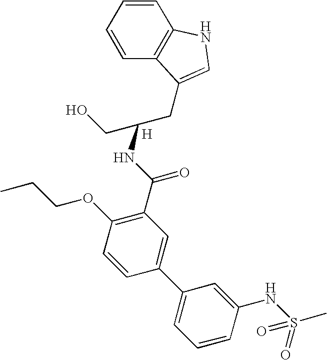 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 495.5 Retention time: 10.5 min. | 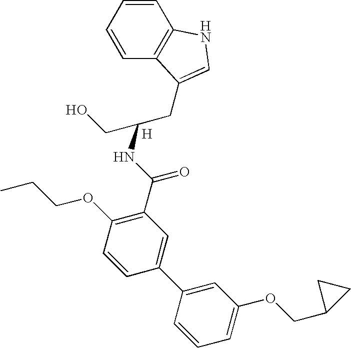 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 507.5 Retention time: 8.45 min. | 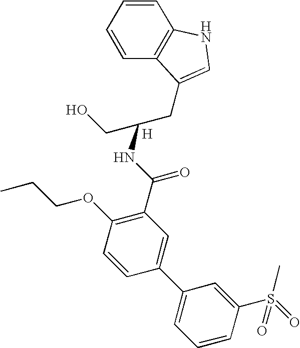 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% on B: 5% to 95%(10′) to 95% (2′) to 5%(0.5′) to 5%(2.5′) Molecular peak(ESI, M + 1): 543.5 Retention time: 6.67 min. | 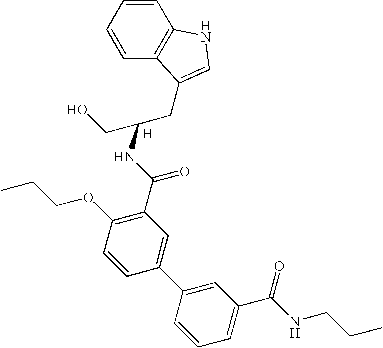 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 517.5 Retention time: 9.13 min. | 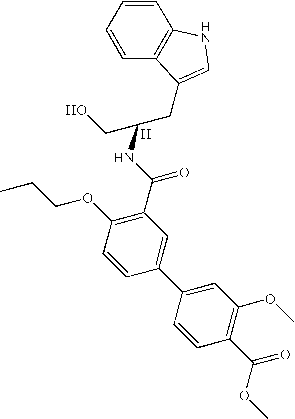 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 507 Retention time: 7.85 min. | 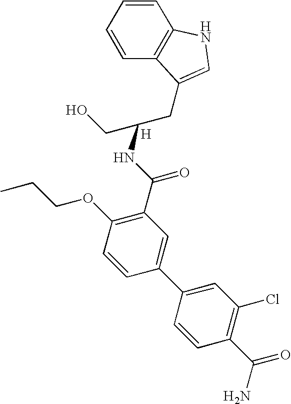 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 536.5 Retention time: 9.13 min. | 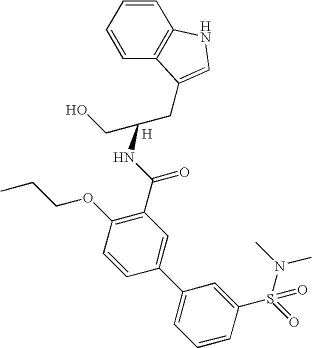 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 535.5 Retention time: 9.03 min. | 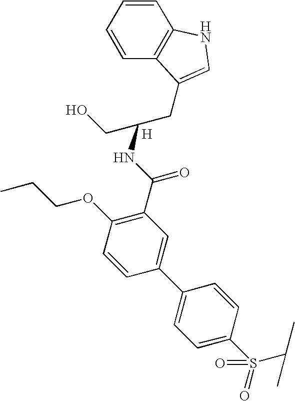 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% on B: 5% to 95%(10′) to 95% (2′) to 5%(0.5′) to 5%(2.5′) Molecular peak(ESI, M + 1): 522.5 Retention time: 8.49 min. | 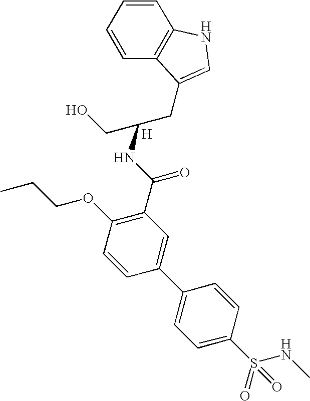 |
| Column Purospher Star RP C18 4.6 × 125 5 μm; detection waveiength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 536.5 Retention time: 9.1 min. | 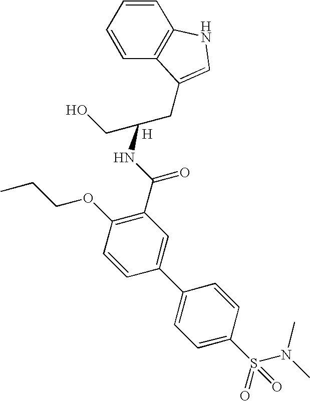 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 472.5 Retention time: 7.59 min. | 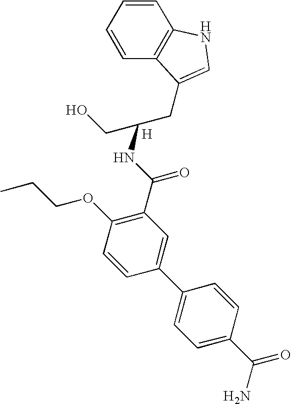 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 522.5 Retention time: 8.57 min. | 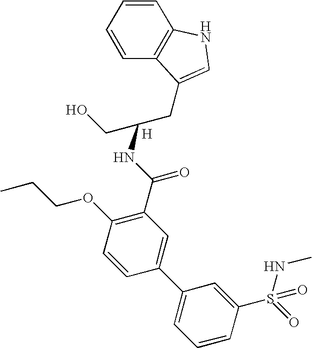 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 521.6 Retention time: 9.22 min. | 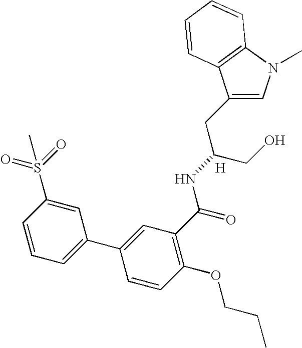 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% on B: 5% to 95%(10′) to 95% (2′) to 5%(0.5′) to 5%(2.5′) Molecular peak(ESI, M + 1): 531.6 Retention time: 9.92 min. | 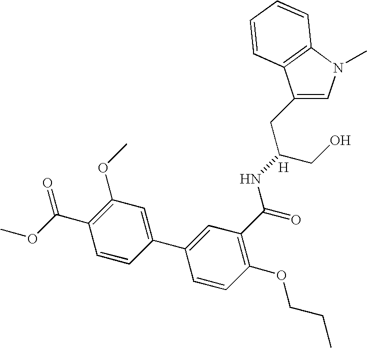 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 557.7 Retention time: 6.91 min. | 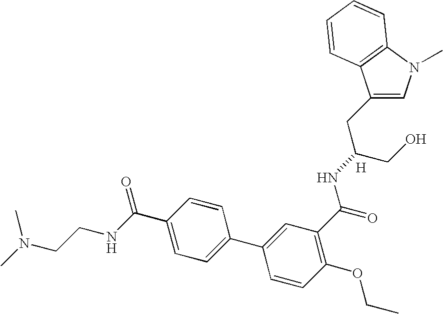 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 491.5 Retention time: 8.37 min. | 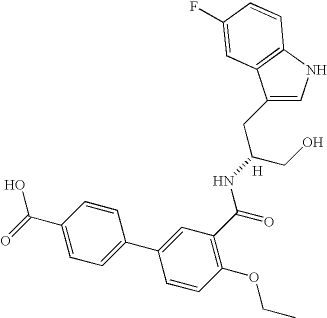 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 540.6 Retention time: 8.57 min. | 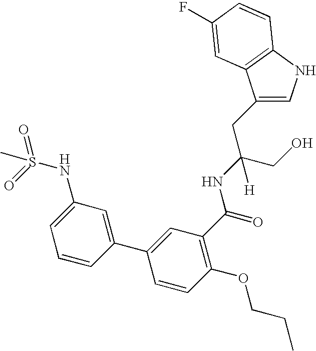 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 525.6 Retention time: 8.61 min. | 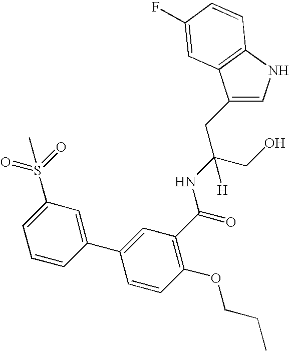 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 561.7 Retention time: 6.85 min. | 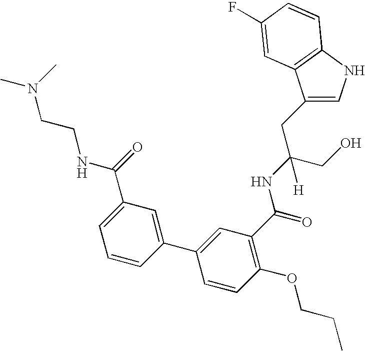 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 525 Retention time: 7.99 min. | 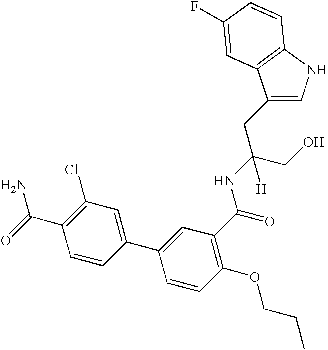 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 554.7 Retention time: 9.17 min. | 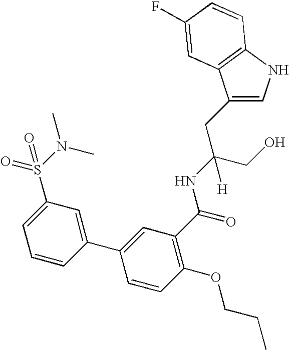 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 553.7 Retention time: 9.18 min. | 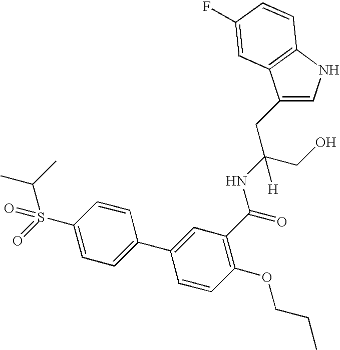 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 554.7 Retention time: 9.12 min. | 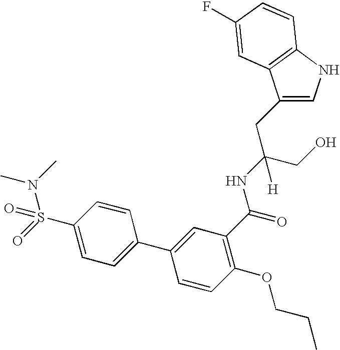 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 546.7 Retention time: 9.07 min. | 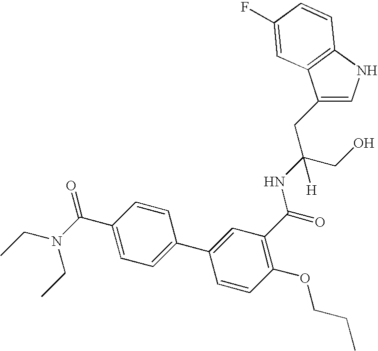 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 540.6 Retention time: 8.72 min. | 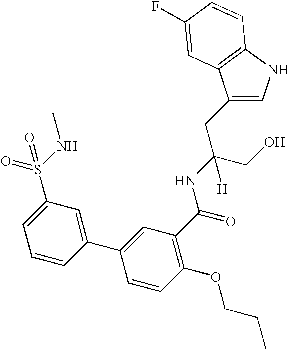 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% on B: 5% to 95%(10′) to 95% (2′) to 5%(0.5′) to 5%(2.5′) Molecular peak(ESI, M + 1): 485.6 Retention time: 9.8 min. | 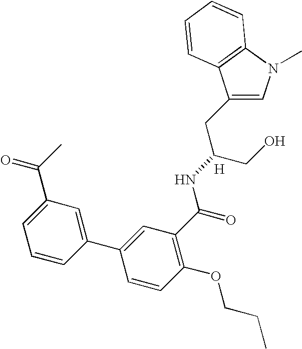 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 519.7 Retention time: 11.62 min. | 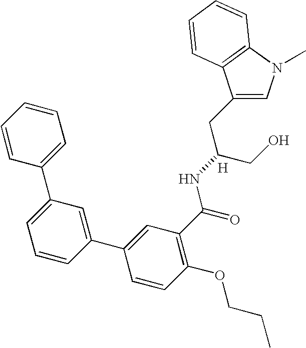 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 482.6 Retention time: 9.81 min. | 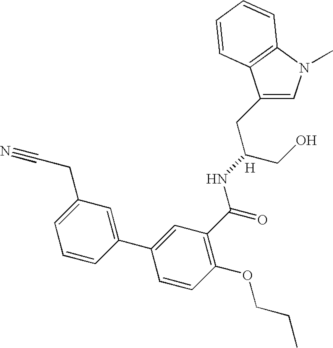 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 436.7 Retention time: 9.1 min. | 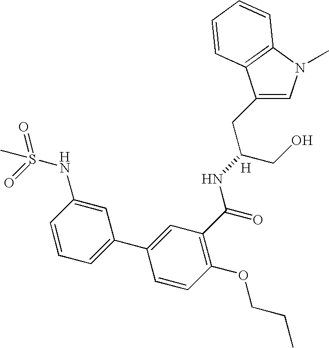 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 482.6 Retention time: 9.67 min. | 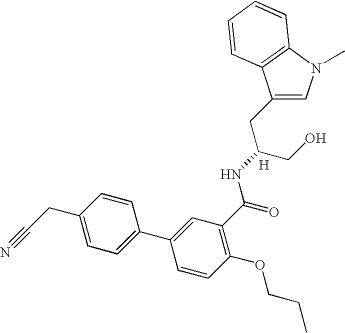 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% on B: 5% to 95%(10′) to 95% (2′) to 5%(0.5′) to 5%(2.5′) Molecular peak(ESI, M-′-1): 557.7 Retention time: 7.19 min. | 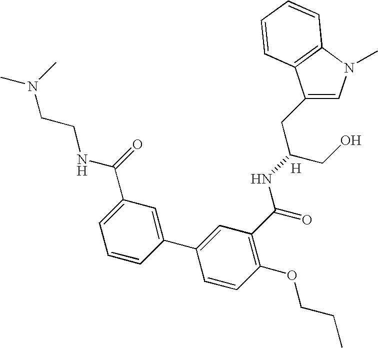 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 505.6 Retention time: 8.94 min. | 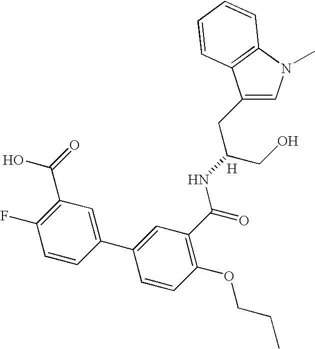 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 521 Retention time: 8.44 min. | 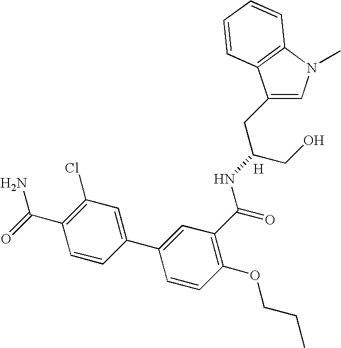 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 542.7 Retention time: 9.7 min. | 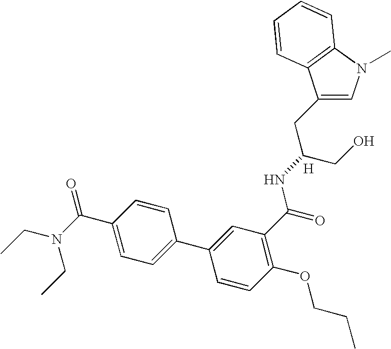 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 490.6 Retention time: 6.91 min. | 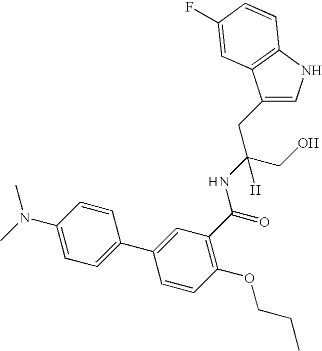 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% on B: 5% to 95%(10′) to 95% (2′) to 5%(0.5′) to 5%(2.5′) Molecular peak(ESI, M + 1): 489.6 Retention time: 9.28 min. | 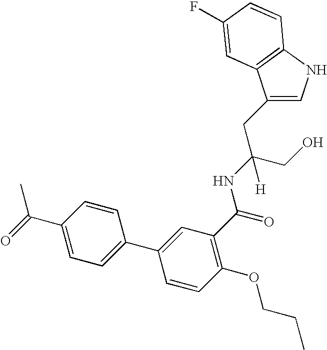 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 489.6 Retention time: 9.33 min. | 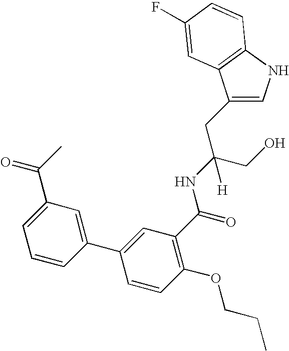 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 523.6 Retention time: 10.86 min. | 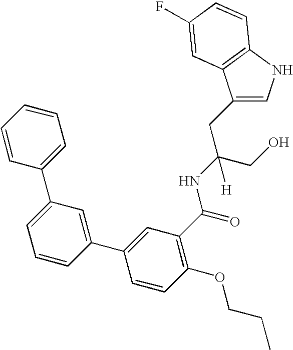 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 535.6 Retention time: 9.16 min. | 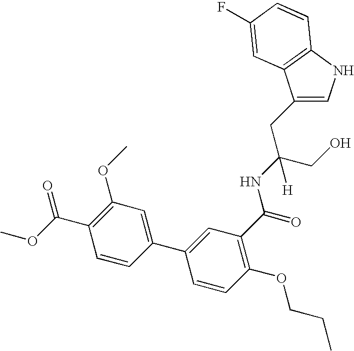 |
| Column Purospher Star RP C18 4.6 × 125 5 μm; detection wavelength 214 nm; flow rate 1 ml/min; eluents A: 0.1% TFA in H₂O, B 0.1% TFA in ACN; gradient in each case based on B: 5% to 95%(10′) to 95% (2′) to 5%(0.5′) to 5%(2.5′) Molecular peak(ESI, M + 1): 490.5 Retention time: 7.64 min. | 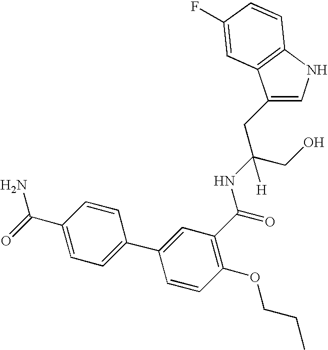 |
| (DMSO-d₆): 10.79 s(1H); 8.36 δ(J=8.1Hz, 1H); 8.14d (J=2.5 Hz, 1H); 7.73dd(J=2.8 Hz/8.6 Hz, 1H); 7.68d(J=7.8 Hz, 2H); 7.59 δ(J=8.3 Hz, 2H); 7.36 δ(J=8.3 Hz, 2H); 7.29 δ(J=8.1Hz, 1H); 2H); 7.29 δ(J=8.1Hz, 1H); 7.19d(J8.8 Hz, 1H);7.13s (1H); 7.03m(1H); 6.94m (1H); 4.90m(1H); 4.41s (2H); 4.23m(1H); 4.12m (2H); 3.47m(1H); 3.41m (1H); 2.95m(2H), 1.28m (3H). | 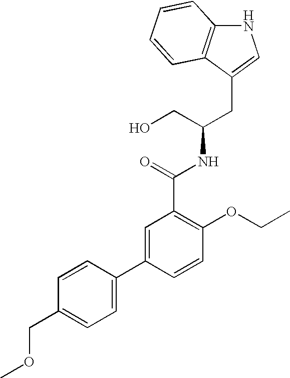 |
| (DMSO-d₆): 10.78 s(1H); 8.37 δ(J=8.1Hz, 1H); 8.21d (J=2.6 Hz, 1H); 8.11 S(2H); 7.79m(2H); 7.69 δ(J=7.9 Hz, 1H); 7.50m(1H); 7.40s (1H); 7.31 δ(J=8.1Hz; 1H); 7.23 δ(J=8.7 Hz, 1H); 7.14s (1H); 7.02m(1H); 6.94m (1H); 4.90m(1H); 4.18m (1H); 4.14m(2H); 3.47m (2H); 2.96m(2H); 1.29m (3H). | 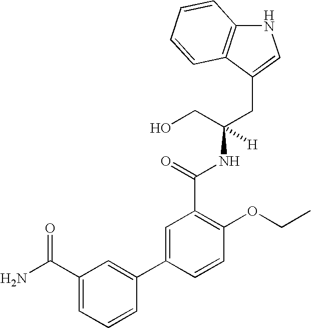 |
| (DMSO-d₆): 10.78 s(1H); 8.35 δ(J=8.1Hz, 1H); 8.20d (J=2.5 Hz, 1H); 7.91 s(4H); 7.85dd(J=2.5 Hz I 8.6 Hz, 1H); 7.67 δ(J=7.8 Hz, 1H); 7.30 δ(J=8.1Hz, 1H); 7.24d (J=8.8 Hz, 1H); 7.14 s(1H); 7.03m(1H); 6.93m(1H); 4.90m(1H); 4.23m(2H); 4.15m(2H); 4.00m(1H); 3.47m(1H); 3.41m(1H); 2.96m(2H); 1.29m(3H); 1.10m(3H). | 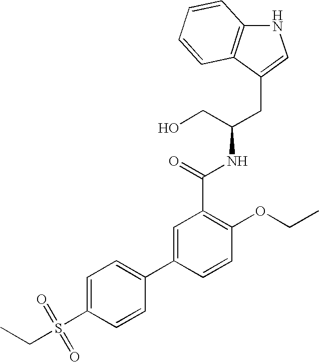 |
| (DMSO-d₆): 10.79 s(1H); 8.37 δ(J=8.3 Hz, 1H); 8.18d (J=2.5 Hz, 1H); 7.79dd(J=2.6 Hz/7.8 Hz, 1H); 7.72d(J=8.5 Hz, 2H); 7.67 δ(J=7.7 Hz, 1H); 7.53 δ(J=8.3 Hz, 1H); 7.29 δ(J=8.1Hz, 1H); 7.22 δ(J=8.2 Hz, 1H); 7.13 s (1H); 7.03m(1H); 6.93m (1H); 4.22m(1H); 4.13m (2H); 3.57m(6 H, broad), 3.43m(2H); 3.09m(2H); 2.95m(2H); 2.80 s(3H); 1.29 m(3H). | 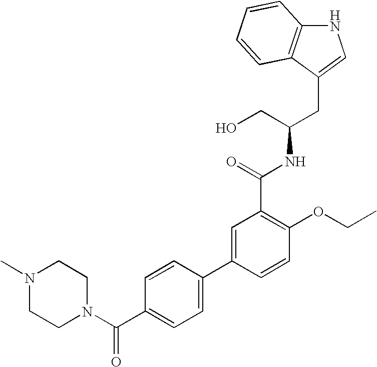 |
| (DMSO-d₆): 8.30 δ(J=2.5 Hz, 1H); 7.75dd(J=2.5 Hz/8.7 Hz, 1H); 7.69 δ(J=7.9 Hz, 1H); 7.35m(2H); 7.17m (4H);7. 11m(1H);6.99m (1H); 6.93m(1H); 4.53m (1H); 4.16m(1H); 4.05m (1H); 3.92 δ(J=7.0 Hz, 1H); 3.70 δ(J=4.9 Hz, 1H); 3.17d (J=6.4 Hz, 1H); 1.27m(4H); 0.67m(2H); 0.41m(2H). | 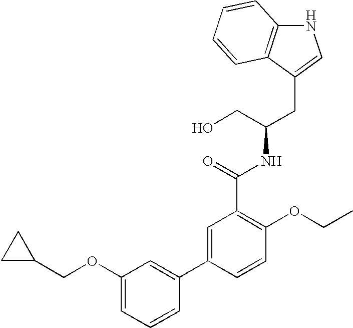 |
| (DMSO-d₆): 10.72s(1H); 8.21 δ(J=8.1Hz, 1H); 7.77 m(3H); 7.63m(2H); 7.44m (3H); 7.36m(1H); 7.26 δ(J=8.1Hz, 1H); 7.09s(1H); 7.01 m(1H); 6.92m(1H); 4.76m (1H); 4.23m(1H); 3.52s (3H); 3.49m(2H); 2.97m (1H); 2.88m(1H). | 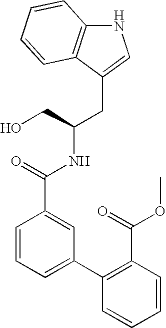 |

1. Compounds of the formula I 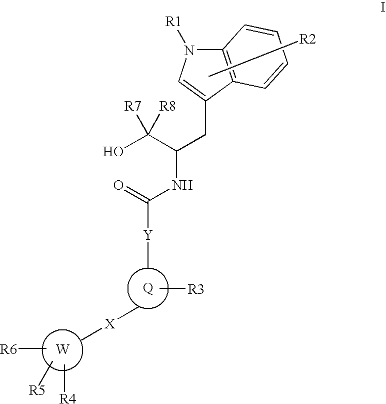
in which
R1 may be hydrogen, C₁-C₆-alkyl, C₃-C₆-alkenyl, C₃-C₆-alkynyl, C₃-C₇-cycloalkyl, C₁-C₆-alkyloxy-C₁-C₆-alkylene, C₃-C₇-cycloalkyloxy-C₁-C₆-alkylene, C₁-C₆-alkylamino-C₁-C₆-alkylene, di(C₁-C₆-alkyl)amino-C₁-C₆-alkylene, phenyloxy-C₁-C₆-alkylene;
where the hydrocarbon chains therein may optionally be substituted one or more times by fluorine, cyano, hydroxy, amino or the groups: 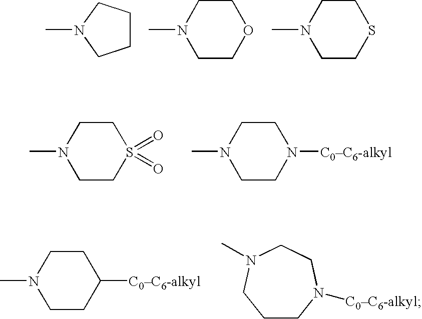
R2 may be hydrogen, halogen, cyano, —SO₂Me, C₁-C₆-alkyl, C₂-C₆-alkenyl, C₂-C₆-alkynyl, C₁-C₆-alkyloxy or benzyloxy,
where the hydrocarbon chains therein may optionally be substituted one or more times by fluorine;
R3 may be hydrogen, hydroxy, halogen, nitro, amino, cyano, C₁-C₆-alkyl, C₂-C₆-alkenyl or C₂-C₆-alkynyl, C₃-C₇-cycloalkyl, hydroxy-C₁-C₆-alkylene, hydroxy-C₃-C₆-alkenylene, hydroxy-C₃-C₆-alkynylene, C₁-C₆-alkyloxy, C₁-C₆-alkyloxy-C₁-C₆-alkylene, C₃-C₇-cycloalkyloxy, C₃-C₇-cycloalkyl-C₁-C₆-alkyleneoxy, C₃-C₇-cycloalkyloxy-C₁-C₆-alkylene, C₁-C₆-alkyloxy-C₃-C₆-alkenylene, C₁-C₆-alkyloxy-C₃-C₆-alkynylene, C₁-C₆-alkyloxyphenyl-C₁₋₆-alkylene, C₁-C₆-alkylamino-C₁-C₆-alkylene, di(C₁-C₆-alkyl)amino-C₁-C₆-alkylene, phenyloxy-C₁-C₆-alkylene;
where the hydrocarbon chains therein may optionally be substituted one or more times by fluorine, cyano, hydroxy, amino or the groups 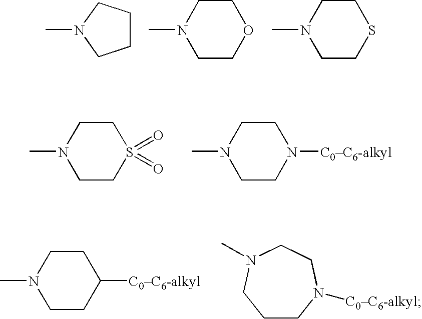
R4, R5, R6 may be independently of one another hydrogen, hydroxy, halogen, nitro, amino, cyano, phenyl, C₁-C₆-alkyl, C₂-C₆-alkenyl or C₂-C₆-alkynyl, C₃-C₇-cycloalkyl, C₃-C₇-cycloalkyl-C₁-C₆-alkylene, C₃-C₇-heterocycloalkyl, where the hydrocarbon chains therein may optionally be substituted one or more times by fluorine, cyano or the radicals: 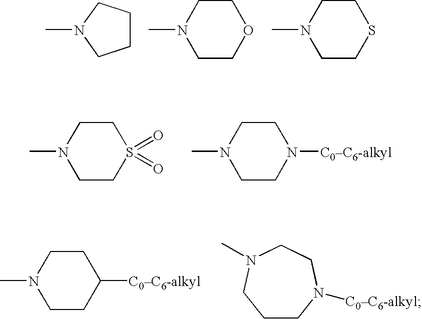
or
independently of one another hydroxy-C₁-C₆-alkylene, hydroxy-C₃-C₆-alkenylene, hydroxy-C₃-C₆-alkynylene, C₁-C₆-alkyloxy, C₃-C₇-cycloalkyloxy, C₃-C₇-cycloalkyl-C₁₋₆-alkyleneoxy, C₁-C₆-alkyloxy-C₁-C₆-alkylene, C₃-C₇-cycloalkyloxy-C₁-C₆-alkylene, C₁-C₆-alkyloxy-C₃-C₆-alkenylene, C₁-C₆-alkyloxy-C₃-C₆-alkynylene, C₁-C₆-alkyloxyphenyl-C₁-C₆-alkylene, phenyloxy-C₁-C₆-alkylene, C₁-C₆-alkylamino, di(C₁-C₆-alkyl)amino, C₁-C₆-alkylamino-C₁-C₆-alkylene, di(C₁-C₆)-alkylamino-C₁-C₆-alkylene, C₃-C₇-cycloalkyl-(C₀-C₆-alkyl)amino, C₁-C₆-acyl-(C₀-C₆-alkyl)amido, C₁-C₆-alkylaminocarbonyl, di(C₁₋₆-alkyl)aminocarbonyl, (C₃-C₇-cycloalkyl)aminocarbonyl, di(C₃-C₇-cycloalkyl)aminocarbonyl, C₃-C₇-cycloalkyl-C₁-C₆-alkyleneaminocarbonyl, C₁-C₆-alkylcarbonyl, C₃-C₇-cycloalkylcarbonyl, carboxy, carboxamido [—C(O)NH₂], C₁-C₆-alkyloxycarbonyl, C₁-C₃-alkylsulphanyl, C₁-C₆-alkysulphonyl, C₃₋₇-cycloalkylsulphonyl, C₃-C₇-cycloalkyl-C₁-C₆-alkylenesulphonyl, C₁-C₆-alkylaminosulphonyl, di(C₁-C₆-alkyl)aminosulphonyl, (C₃-C₇-cycloalkyl)aminosulphonyl, di(C₃-C₇-cycloalkyl)aminosulphonyl, C₃-C₇-cycloalkyl-C₁-C₆-alkyleneaminosulphonyl, C₁-C₆-alkylsulphonylamido, —N(C₀₋₆-alkyl)-C(O)—C₁-C₆-alkyl, —N(C₀-C₆-alkyl)-C(O)—C₃-C₇-cycloalkyl, —N(C₀-C₆-alkyl)-C(O)—N-di(C₀-C₆-alkyl), —N(C₀-C₆-alkyl)-C(O)—O—(C₀-C₆)alkyl, —N(C₀-C₆-alkyl)-C(O)—NH—C₃-C₇-cycloalkyl, —N(C₀-C₆-alkyl)-SO₂—C₁₋₆-alkyl, —N(C₀-C₆-alkyl)-SO₂—C₃-C₇-cycloalkyl, —N(C₀-C₆-alkyl)-SO₂—N-di(C₀-C₆-alkyl), —N(C₀-C₆-alkyl)-SO₂—NH—(C₃-C₇)-cycloalkyl, —C(O)—N(H)—C₂-C₆-alkylene-(C₁-C₆-alkyl)amine, —C(O)—N(H)—C₂-C₆-alkylene-[di(C₁-C₆-alkyl)]amine, —C(O)—N(H)—C₂-C₆-alkylene-(C₃-C₇-cycloalkyl)amine, —C(O)—N(H)—C₂-C₆-alkylene-(C₃-C₇-cycloalkyl-C₁-C₆-alkyl)amine, —S(O₂)—N(H)—C₂-C₆-alkylene-(C₁-C₆-alkyl)amine, —S(O₂)—N(H)—C₂-C₆-alkylene-[di(C₁-C₆-alkyl)]amine, —S(O₂)—N(H)—C₂-C₆-alkylene-(C₃-C₇-cycloalkyl)amine, —S(O₂)—N(H)—C₂-C₆-alkylene-(C₃-C₇-cycloalkyl-C₁-C₆-alkylene)amine, —O—C₂-C₆-alkylene-(C₁-C₆-alkyl)amine, —O—C₂-C₆-alkylene-[di(C₁-C₆-alkylene)]amine,
or the radicals: 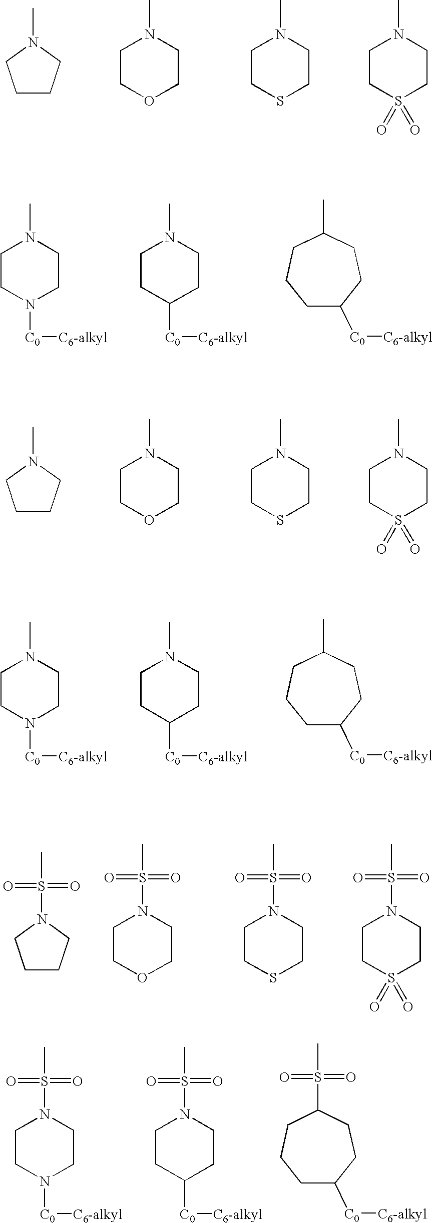 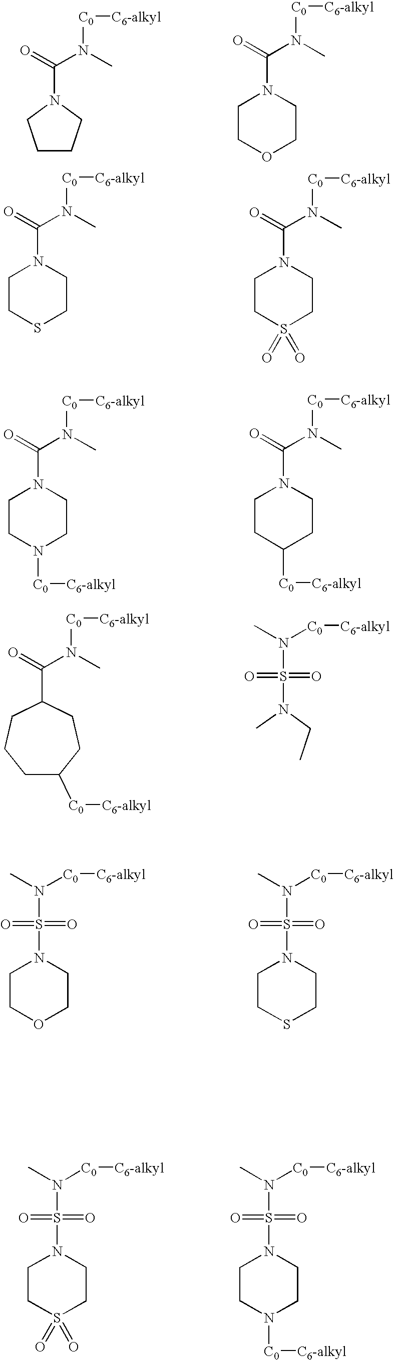 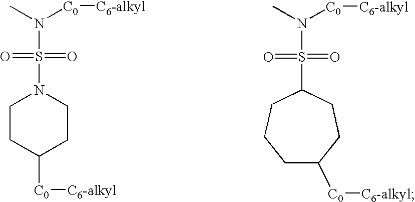
R7, R8 may be independently of one another hydrogen, methyl, ethyl, where the methyl and ethyl radicals may be fluorinated one or more times;
where
R2 may substitute one or more positions of the aryl or heteroaryl ring in the indole residue;
R3 may substitute one or more positions of the aryl or heteroaryl ring in the radical Q;
R5 and R6 may together form heterocycloalkyl, cycloalkyl;
Q and W may be independently of one another aryl, heteroaryl;
X may be a bond, C₁₋₄-alkylene, C₂-C₄-alkenylene, C₂-C₄-alkynylene, C₁-C₃-alkyleneoxy, C₁-C₃-alkyleneoxy-C₁-C₃-alkylene,
Y may be a bond, C₁-C₄-alkylene.

2. Compounds of the formula Ia 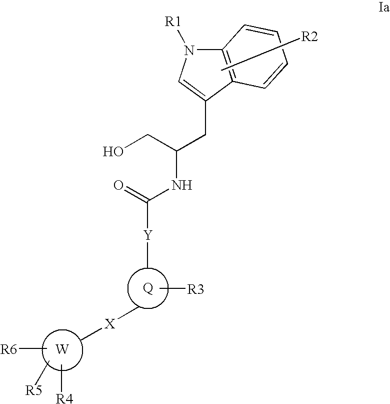
where
R1 may be hydrogen, C₁-C₆-alkyl, C₃-C₆-alkenyl or C₃-C₆-alkynyl, where the hydrocarbon radicals therein may optionally be substituted one or more times by fluorine;
R2 may be hydrogen, halogen, C₁-C₆-alkyl, C₂-C₆-alkenyl or C₂-C₆-alkynyl, C₁-C₄-alkyloxy, where the hydrocarbon chain therein may optionally be substituted one or more times by fluorine; or benzyloxy;
R3 may be hydrogen, halogen, nitro, amino, cyano, C₁-C₆-alkyl, C₂-C₆-alkenyl or C₂-C₆-alkynyl, C₁-C₄-alkykoxy, where the hydrocarbon chain therein may optionally be substituted one or more times by fluorine;
R4, R5, R6 may be independently of one another hydrogen, halogen, C₁-C₆-alkyl, C₂-C₆-alkenyl, C₂-C₆-alkynyl, C₁-C₄-alkyloxy, where the hydrocarbon chain therein may optionally be substituted one or more times by fluorine, C₁-C₃-alkylsulphanyl, acetamido, C₁-C₆-alkylaminocarbonyl; hydroxy, cyano, hydroxy-C₁-C₄-alkyl;
where
R2 and R3 may substitute one or more positions of the aryl or heteroaryl ring in each case in the radical Q and in the indole residue;
R5 and R6 may together form heterocycloalkyl, cycloalkyl;
Q and W may be independently of one another aryl, heteroaryl;
X may be a bond, C₁-C₄-alkylene, C₁-C₄-alkenylene, C₁-C₄-alkynylene, C₁-C₃-alkyleneoxy, C₁₋₃-alkyleneoxy-C₁-C₃-alkylene,
Y may be a bond, C₁-C₄-alkylene.

3. Compounds according to claim 1, namely acyltryptophanols of the formulae II and III 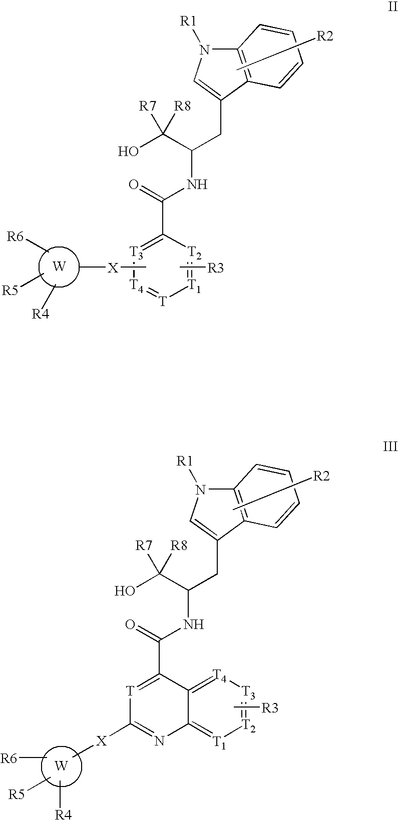
in which the radicals R1 to R8 and W have the same meaning as in formula I and
X is a bond, C₁-C₄-alkylene, C₂-C₄-alkenylene, C₂-C₄-alkynylene;
T is a nitrogen atom or a CH group;
T1, T2, T3, T4 are each independently of one another a nitrogen atom or an R3-C group.

4. Compounds according to claim 2, namely acyltryptophanols of the formulae IIa and IIIa 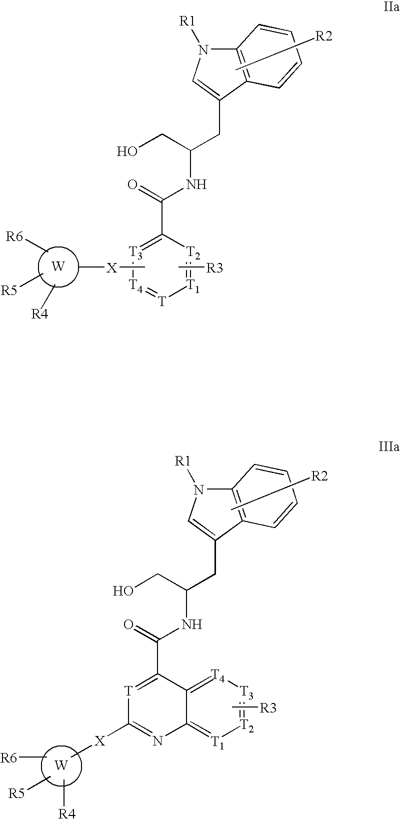
in which the radicals R1 to R6 and W have the same meaning as in formula Ia and
X is a bond, C₁-C₄-alkylene, C₂-C₄-alkenylene, C₂-C₄-alkynylene;
T is a nitrogen atom or a CH group;
T1, T2, T3, T4 are each independently of one another a nitrogen atom or an R3-C group.

5. Compounds according to claim 1 or 3, namely acyltryptophanols of the formulae IV and V 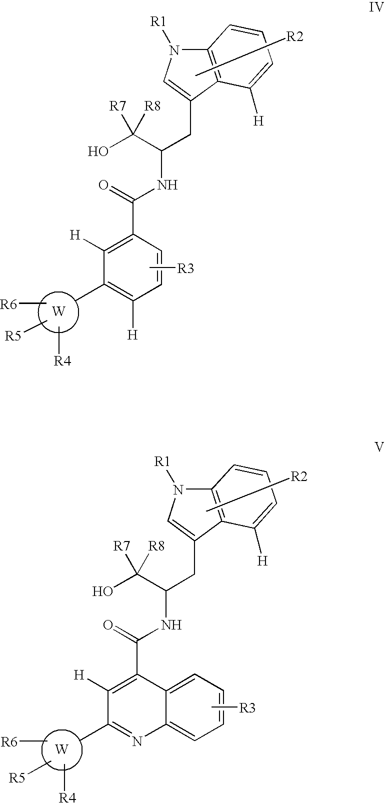
in which the radicals R1 to R8 and W have the same meaning as in formula I.

6. Compounds according to claim 2 on, namely acyltryptophanols of the formulae IVa and Va 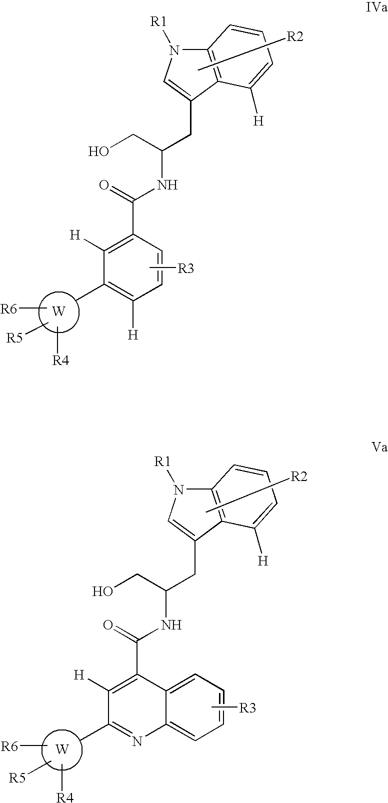
in which the radicals R1 to R6 and W have the same meaning as in formula Ia.

7. Compounds according to claim 1, namely
N-[(R,S)-2-(5-Bromo-1H-indol-3-yl)-1-(hydroxymethyl)ethyl]-2-(3,4,5-trimethoxyphenyl)quinoline-4-carboxamide;
N-[(R,S)-1-(Hydroxymethyl)-2-(5-methyl-1H-indol-3-yl)ethyl]-2-(3,4,5-trimethoxyphenyl)quinoline-4-carboxamide;
N-[(R,S)-1-(Hydroxymethyl)-2-(4-methyl-1H-indol-3-yl)ethyl]-2-(3,4,5-trimethoxyphenyl)quinoline-4-carboxamide;
N-[(R,S)-1-(Hydroxymethyl)-2-(6-methyl-1H-indol-3-yl)ethyl]-2-(3,4,5-trimethoxyphenyl)quinoline-4-carboxamide;
N-[(R)-1-(Hydroxymethyl)-2-(1-methyl-1H-indol-3-yl)ethyl]-2-(3,4,5-trimethoxyphenyl)quinoline-4-carboxamide;
N-[(R,S)-2-(5-Fluoro-1H-indol-3-yl)-1-(hydroxymethyl)ethyl]-2-(3,4,5-trimethoxyphenyl)quinoline-4-carboxamide;
N-[(R,S)-1-(Hydroxymethyl)-2-(5-methoxy-1H-indol-3-yl)ethyl]-2-(3,4,5-trimethoxyphenyl)quinoline-4-carboxamide;
N-[(R,S)-2-(6-Fluoro-1H-indol-3-yl)-1-(hydroxymethyl)ethyl]-2-(3,4,5-trimethoxyphenyl)quinoline-4-carboxamide;
N-[(R,S)-1-(Hydroxymethyl)-2-[5-(phenylmethoxy)-1H-indol-3-yl]ethyl]-2-(3,4,5-trimethoxyphenyl)quinoline-4-carboxamide;
N-[(R,S)-1-(Hydroxymethyl)-2-(7-methyl-1H-indol-3-yl)ethyl]-2-(3,4,5-trimethoxyphenyl)quinoline-4-carboxamide;
N-[(R)-1-(Hydroxymethyl)-2-(1H-indol-3-yl)ethyl]-2-(3,4,5-trimethoxyphenyl)quinoline-4-carboxamide;
N-[(S)-1-(Hydroxymethyl)-2-(1H-indol-3-yl)ethyl]-2-(3,4,5-trimethoxyphenyl)quinoline-4-carboxamide;
2-(4-Chloro-3-methylphenyl)-N-[(R)-1-(hydroxymethyl)-2-(1H-indol-3-yl)ethyl]quinoline-4-carboxamide
N-[(R)-1-(Hydroxymethyl)-2-(1H-indol-3-yl)ethyl]-6-methoxy-2-(3,4,5-trimethoxyphenyl)quinoline-4-carboxamide;
6-Bromo-N-[(R)-1-(hydroxymethyl)-2-(1H-indol-3-yl)ethyl]-2-(3,4,5-trimethoxyphenyl)quinoline-4-carboxamide;
N-[(R)-1-(Hydroxymethyl)-2-(1H-indol-3-yl)ethyl]-6-methoxy-2-(2,3,4-trimethoxyphenyl)quinoline-4-carboxamide;
6-Fluoro-N-[(R)-1-(hydroxymethyl)-2-(1H-indol-3-yl)ethyl]-2-(3,4,5-trimethoxyphenyl)quinoline-4-carboxamide;
N-[(R)-1-(Hydroxymethyl)-2-(1H-indol-3-yl)ethyl]-6-iodo-2-(3,4,5-trimethoxyphenyl)quinoline-4-carboxamide;
N-[(R)-1-(Hydroxymethyl)-2-(1H-indol-3-yl)ethyl]-6-nitro-2-(3,4,5-trimethoxyphenyl)quinoline-4-carboxamide;
6-Amino-N-[(R)-1-(hydroxymethyl)-2-(1H-indol-3-yl)ethyl]-2-(3,4,5-trimethoxyphenyl)quinoline-4-carboxamide;
N-[(R,S)-1-(Hydroxymethyl)-2-(5-fluoro-1H-indol-3-yl)ethyl]-6-methoxy-2-(3,4,5-trimethoxyphenyl)quinoline-4-carboxamide;
N-[(R)-1-(Hydroxymethyl)-2-(1-methyl-1H-indol-3-yl)ethyl]-6-methoxy-2-(3,4,5-trimethoxyphenyl)quinoline-4-carboxamide;
N-[(R,S)-2-(6-Fluoro-1H-indol-3-yl)-1-(hydroxymethyl)ethyl]-6-methoxy-2-(3,4,5-trimethoxyphenyl)quinoline-4-carboxamide;
2-(3,4-Dimethoxyphenyl)-N-[(S)-1-(hydroxymethyl)-2-(1H-indol-3-yl)ethyl]quinoline-4-carboxamide;
2-(3,4-Dimethoxyphenyl)-N-[(R)-1-(hydroxymethyl)-2-(1H-indol-3-yl)ethyl]quinoline-4-carboxamide;
2-(3,4-Dimethylphenyl)-N-[(R)-1-(hydroxymethyl)-2-(1H-indol-3-yl)ethyl]quinoline-4-carboxamide;
2-(2,3-Dihydro-1,4-benzodioxin-6-yl)-N-[(R)-1-(hydroxymethyl)-2-(1H-indol-3-yl)ethyl]quinoline-4-carboxamide;
N-[(R)-1-(Hydroxymethyl)-2-(1H-indol-3-yl)ethyl]-2-[4-(trifluoromethoxy)phenyl]quinoline-4-carboxamide;
N-[(R)-1-(Hydroxymethyl)-2-(1H-indol-3-yl)ethyl]-2-[4-(methylsulphanyl)phenyl]quinoline-4-carboxamide;
2-(3,5-Dimethoxyphenyl)-N-[(R)-1-(hydroxymethyl)-2-(1H-indol-3-yl)ethyl]quinoline-4-carboxamide;
2-[3-(Acetylamino)phenyl]-N-[(R)-1-(hydroxymethyl)-2-(1H-indol-3-yl)ethyl]quinoline-4-carboxamide;
2-(4-Chlorophenyl)-N-[(R)-1-(hydroxymethyl)-2-(1H-indol-3-yl)ethyl]quinoline-4-carboxamide;
N-[(R)-1-(Hydroxymethyl)-2-(1H-indol-3-yl)ethyl]-2-(4-methoxyphenyl)quinoline-4-carboxamide;
N-[(R)-1-(Hydroxymethyl)-2-(1H-indol-3-yl)ethyl]-2-(3-methoxyphenyl)quinoline-4-carboxamide;
N-[(R)-2-(1-Ethyl-1H-indol-3-yl)-1-(hydroxymethyl)ethyl]-6-methoxy-2-(3,4,5-trimethoxyphenyl)quinoline-4-carboxamide;
2-(2,3-Dihydrobenzofuran-5-yl)-N-[(R)-1-(hydroxymethyl)-2-(1H-indol-3-yl)ethyl]-quinoline-4-carboxamide;
N-[(R)-1-(Hydroxymethyl)-2-(1H-indol-3-yl)ethyl]-6-methoxy-2-(7-methoxybenzofuran-2-yl)quinoline-4-carboxamide;
2-[(Z)-2-(3,4-Dimethoxyphenyl)ethenyl]-N-[(R)-1-(hydroxymethyl)-2-(1H-indol-3-yl)ethyl]-6-methoxyquinoline-4-carboxamide;
N-[(R)-1-(Hydroxymethyl)-2-(1H-indol-3-yl)ethyl]-4′-methoxy[1,1′-biphenyl]-2-carboxamide;
N-[(S)-1-(Hydroxymethyl)-2-(1H-indol-3-yl)ethyl]-3′,4′-dimethoxy[1,1′-biphenyl]-3-carboxamide;
N-[(R)-1-(Hydroxymethyl)-2-(1H-indol-3-yl)ethyl]-3′,4′-dimethoxy[1,1′-biphenyl]-3-carboxamide;
N-[(R)-1-(Hydroxymethyl)-2-(1H-indol-3-yl)ethyl]-2′,3′,4′-trimethoxy[1,1′-biphenyl]-3-carboxamide;
N-[(R)-1-(Hydroxymethyl)-2-(1H-indol-3-yl)ethyl]-3′,4′,5′-trimethoxy[1,1′-biphenyl]-3-carboxamide;
N-[(R)-1-(Hydroxymethyl)-2-(1H-indol-3-yl)ethyl]-3′,4′,5′-trimethoxy[1,1′-biphenyl]-4-carboxamide;
N-[(R)-1-(Hydroxymethyl)-2-(1H-indol-3-yl)ethyl]-3′,4′,5′-trimethoxy-2-methyl[1,1′-biphenyl]-4-carboxamide;
N-[(R)-1-(Hydroxymethyl)-2-(1H-indol-3-yl)ethyl]-2′,3′,4′-trimethoxy[1,1′-biphenyl]-2-carboxamide;
N-[(R)-1-(Hydroxymethyl)-2-(1H-indol-3-yl)ethyl]-3′,4′,5′-trimethoxy[1,1′-biphenyl]-2-carboxamide;
N-[(R)-1-(Hydroxymethyl)-2-(1H-indol-3-yl)ethyl]-2′,3′,4′-trimethoxy-6-methyl[1,1′-biphenyl]-3-carboxamide;
N-[(R)-1-(Hydroxymethyl)-2-(1H-indol-3-yl)ethyl]-3′,4′,5′-trimethoxy-6-methyl[1,1′-biphenyl]-3-carboxamide;
N-[(R)-1-(Hydroxymethyl)-2-(1H-indol-3-yl)ethyl]-2′,3′,4′-trimethoxy[1,1′-biphenyl]-4-carboxamide;
N-[(R)-1-(Hydroxymethyl)-2-(1H-indol-3-yl)ethyl]-2′,3′,4′-trimethoxy-2-methyl[1,1′-biphenyl]-4-carboxamide;
N-[(R)-1-(Hydroxymethyl)-2-(1H-indol-3-yl)ethyl]-2′,3′,4,4′-tetramethoxy[1,1′-biphenyl]-2-carboxamide;
N-[(R)-1-(Hydroxymethyl)-2-(1H-indol-3-yl)ethyl]-3′,4,4′,5′-tetramethoxy[1,1′-biphenyl]-2-carboxamide;
4′-(Hydroxymethyl)-N-[(R)-1-(hydroxymethyl)-2-(1H-indol-3-yl)ethyl]-6-methyl[1,1′-biphenyl]-3-carboxamide;
4′-(Hydroxymethyl)-N-[(R)-1-(hydroxymethyl)-2-(1H-indol-3-yl)ethyl]-2-methyl[1,1′-biphenyl]-4-carboxamide;
4′-(Hydroxymethyl)-N-[(R)-1-(hydroxymethyl)-2-(1H-indol-3-yl)ethyl][1,1′-biphenyl]-2-carboxamide;
4′-(Hydroxymethyl)-N-[(R)-1-(hydroxymethyl)-2-(1H-indol-3-yl)ethyl][1,1′-biphenyl]-3-carboxamide;
N-[(R)-1-(Hydroxymethyl)-2-(1H-indol-3-yl)ethyl]-4-methoxy-3′-(1-methylethyl)[1,1′-biphenyl]-2-carboxamide;
N-[(R)-1-(Hydroxymethyl)-2-(1H-indol-3-yl)ethyl]-3′-(1-methylethyl)[1,1′-biphenyl]-3-carboxamide;
N-[(R)-1-(Hydroxymethyl)-2-(1H-indol-3-yl)ethyl]-6-methyl-3′-(1-methylethyl)[1,1′-biphenyl]-3-carboxamide;
N-[(R)-1-(Hydroxymethyl)-2-(1H-indol-3-yl)ethyl]-3′-(1-methylethyl)[1,1′-biphenyl]-4-carboxamide;
N-[(R)-1-(Hydroxymethyl)-2-(1H-indol-3-yl)ethyl]-2-methyl-3′-(1-methylethyl)[1,1′-biphenyl]-4-carboxamide;
4′-(Hydroxymethyl)-N-[(R)-1-(hydroxymethyl)-2-(1H-indol-3-yl)ethyl]-4-methoxy[1,1′-biphenyl]-2-carboxamide;
3′,4′,5′-Trifluoro-N-[(R)-1-(hydroxymethyl)-2-(1H-indol-3-yl)ethyl][1,1′-biphenyl]-2-carboxamide;
3′,4′,5′-Trifluoro-N-[(R)-1-(hydroxymethyl)-2-(1H-indol-3-yl)ethyl][1,1′-biphenyl]-3-carboxamide;
3′,4′,5′-Trifluoro-N-[(R)-1-(hydroxymethyl)-2-(1H-indol-3-yl)ethyl]-6-methyl[1,1′-biphenyl]-3-carboxamide;
3′,4′,5′-Trifluoro-N-[(R)-1-(hydroxymethyl)-2-(1H-indol-3-yl)ethyl][1,1′-biphenyl]-4-carboxamide;
3′,4′,5′-Trifluoro-N-[(R)-1-(hydroxymethyl)-2-(1H-indol-3-yl)ethyl]-2-methyl[1,1′-biphenyl]-4-carboxamide;
N-[(R)-1-(Hydroxymethyl)-2-(1H-indol-3-yl)ethyl]-2′,5′-dimethoxy[1,1′-biphenyl]-2-carboxamide;
N-[(R)-1-(Hydroxymethyl)-2-(1H-indol-3-yl)ethyl]-2′,4,5′-trimethoxy[1,1′-biphenyl]-2-carboxamide;
N-[(R)-1-(Hydroxymethyl)-2-(1H-indol-3-yl)ethyl]-2′,5′-dimethoxy-6-methyl[1,1′-biphenyl]-3-carboxamide;
N-[(R)-1-(Hydroxymethyl)-2-(1H-indol-3-yl)ethyl]-2′,5′-dimethoxy[1,1′-biphenyl]-4-carboxamide;
N-[(R)-1-(Hydroxymethyl)-2-(1H-indol-3-yl)ethyl]-2′,5′-dimethoxy-2-methyl[1,1′-biphenyl]-4-carboxamide;
N-[(R)-1-(Hydroxymethyl)-2-(1H-indol-3-yl)ethyl]-3′,4′-dimethoxy[1,1′-biphenyl]-2-carboxamide;
N-[(R)-1-(Hydroxymethyl)-2-(1H-indol-3-yl)ethyl]-3′,4,4′-trimethoxy[1,1′-biphenyl]-2-carboxamide;
N-[(R)-1-(Hydroxymethyl)-2-(1H-indol-3-yl)ethyl]-3′,4′-dimethoxy-6-methyl[1,1′-biphenyl]-3-carboxamide;
N-[(R)-1-(Hydroxymethyl)-2-(1H-indol-3-yl)ethyl]-3′,4′-dimethoxy[1,1′-biphenyl]-4-carboxamide;
N-[(R)-1-(Hydroxymethyl)-2-(1H-indol-3-yl)ethyl]-3′,4′-dimethoxy-2-methyl[1,1′-biphenyl]-4-carboxamide;
3′-Fluoro-N-[(R)-1-(hydroxymethyl)-2-(1H-indol-3-yl)ethyl]-4′-methoxy[1,1′-biphenyl]-2-carboxamide;
3′-Fluoro-N-[(R)-1-(hydroxymethyl)-2-(1H-indol-3-yl)ethyl]-4,4′-dimethoxy[1,1′-biphenyl]-2-carboxamide;
3′-Fluoro-N-[(R)-1-(hydroxymethyl)-2-(1H-indol-3-yl)ethyl]-4′-methoxy[1,1′-biphenyl]-3-carboxamide;
3′-Fluoro-N-[(R)-1-(hydroxymethyl)-2-(1H-indol-3-yl)ethyl]-4′-methoxy-6-methyl[1,1′-biphenyl]-3-carboxamide;
3′-Fluoro-N-[(R)-1-(hydroxymethyl)-2-(1H-indol-3-yl)ethyl]-4′-methoxy[1,1′-biphenyl]-4-carboxamide;
3′-Fluoro-N-[(R)-1-(hydroxymethyl)-2-(1H-indol-3-yl)ethyl]-4′-methoxy-2-methyl[1,1′-biphenyl]-4-carboxamide;
N-[(R)-1-(Hydroxymethyl)-2-(1H-indol-3-yl)ethyl]-3′,4-dimethoxy[1,1′-biphenyl]-2-carboxamide;
N-[(R)-1-(Hydroxymethyl)-2-(1H-indol-3-yl)ethyl]-3′-(1-methylethyl)[1,1′-biphenyl]-2-carboxamide;
N-[(R)-1-(Hydroxymethyl)-2-(1H-indol-3-yl)ethyl]-2′,5′-dimethoxy[1,1′-biphenyl]-3-carboxamide;
3′,4′,5′-Trifluoro-N-[(R)-1-(hydroxymethyl)-2-(1H-indol-3-yl)ethyl]-4-methoxy[1,1′-biphenyl]-2-carboxamide;
3-(Benzofuran-2-yl)-N-[(R)-1-(hydroxymethyl)-2-(1H-indol-3-yl)ethyl]benzamide;
N-[(R)-1-(Hydroxymethyl)-2-(1H-indol-3-yl)ethyl]-3-(5-methoxybenzofuran-2-yl)-benzamide;
N-[(R)-1-(Hydroxymethyl)-2-(1H-indol-3-yl)ethyl]-2-[(3,4,5-trimethoxyphenyl)methoxy]-phenylpropanamide
N-[(R)-1-(Hydroxymethyl)-2-(1H-indol-3-yl)ethyl]-4-[[(3,4,5-trimethoxyphenyl)methoxy]methyl]benzamide;
N-[(R)-1-(Hydroxymethyl)-2-(1H-indol-3-yl)ethyl]-3-[(3,4,5-trimethoxyphenyl)methoxy]-thiophene-2-carboxamide;
N-[(R)-1-(Hydroxymethyl)-2-(1H-indol-3-yl)ethyl]-4-[(3,4,5-trimethoxyphenyl)methoxy]-phenylacetamide;
N-[(R)-1-(Hydroxymethyl)-2-(1H-indol-3-yl)ethyl]-3-[(3,4,5-trimethoxyphenyl)methoxy]-phenylpropanamide;
2-[2-(3,4-Dimethoxyphenyl)ethyl]-N-[(R)-1-(hydroxymethyl)-2-(1H-indol-3-yl)ethyl]-6-methoxyquinoline-4-carboxamide;
N-[(R)-1-(Hydroxymethyl)-2-(1H-indol-3-yl)ethyl]-2-(3,4,5-trimethoxyphenyl)-1,6-naphthyridine-4-carboxamide;
6-Bromo-N-[(R)-1-(hydroxymethyl)-2-(1H-indol-3-yl)ethyl]-2-(3,4,5-trimethoxyphenyl)-1,8-naphthyridine-4-carboxamide;

8. Compounds according to claim 1, namely
2-(6-Methoxynaphthalen-2-yl)quinoline-4-carboxylic acid [(R)-1-hydroxymethyl-2-(1H-indol-3-yl)ethyl]amide;
6-Methoxy-2-(3-methoxyphenyl)quinoline-4-carboxylic acid [(R)-1-hydroxymethyl-2-(1H-indol-3-yl)ethyl]amide;
2-(4-Fluoro-3-methoxyphenyl)-6-methoxyquinoline-4-carboxylic acid [(R)-2-hydroxy-1-(1H-indol-3-ylmethyl)ethyl]amide;
2-(3-Iodo-4-methoxyphenyl)-6-methoxyquinoline-4-carboxylic acid [(R)-2-hydroxy-1-(1H-indol-3-ylmethyl)ethyl]amide;
2-(3-Hydroxyphenyl)-6-methoxyquinoline-4-carboxylic acid [(R)-1-hydroxymethyl-2-(1H-indol-3-yl)ethyl]amide;
2-(4-Hydroxy-3,5-dimethoxyphenyl)-6-methoxyquinoline-4-carboxylic acid [(R)-1-hydroxymethyl-2-(1H-indol-3-yl)ethyl]amide;
2-(3,5-Difluoro-4-methoxyphenyl)-6-methoxyquinoline-4-carboxylic acid [(R)-2-hydroxy-1-(1H-indol-3-ylmethyl)ethyl]amide;
2-(3-Ethyl-phenyl)-6-methoxyquinoline-4-carboxylic acid [(R)-1-hydroxymethyl-2-(1H-indol-3-yl)ethyl]amide;
2-(3-Fluoro-4-methoxyphenyl)-6-methoxyquinoline-4-carboxylic acid [(R)-2-hydroxy-1-(1H-indol-3-ylmethyl)ethyl]amide;
2-(3-Fluoro-4-methoxyphenyl)-6-methylquinoline-4-carboxylic acid [(R)-2-hydroxy-1-(1H-indol-3-ylmethyl)ethyl]amide;
6-Methyl-2-(3,4,5-trimethoxyphenyl)quinoline-4-carboxylic acid [(R)-1-hydroxymethyl-2-(1H-indol-3-yl)ethyl]amide;
6-Bromo-2-(2,4-dimethylthiazol-5-yl)quinoline-4-carboxylic acid [(R)-2-hydroxy-1-(1H-indol-3-ylmethyl)ethyl]amide;
2-(7-Methoxybenzofuran-2-yl)-6-trifluoromethoxyquinoline-4-carboxylic acid [(R)-2-hydroxy-1-(1H-indol-3-ylmethyl)ethyl]amide;
2-(3-Fluoro-4-methoxyphenyl)-6-iodoquinoline-4-carboxylic acid [(R)-2-hydroxy-1-(1H-indol-3-ylmethyl)ethyl]amide;
2-(3-Fluoro-4-methoxyphenyl)-6-trifluoromethoxyquinoline-4-carboxylic acid [(R)-2-hydroxy-1-(1H-indol-3-ylmethyl)ethyl]amide;
2-(3-Fluoro-4-methoxyphenyl)-6,8-dimethylquinoline-4-carboxylic acid [(R)-2-hydroxy-1-(1H-indol-3-ylmethyl)ethyl]amide;
2-(3,4-Dimethoxyphenyl)-6-methoxy-3-methylquinoline-4-carboxylic acid [(R)-1-hydroxymethyl-2-(1H-indol-3-yl)ethyl]amide;
6-Amino-2-(3-fluoro-4-methoxyphenyl)quinoline-4-carboxylic acid [(R)-2-hydroxy-1-(1H-indol-3-ylmethyl)ethyl]amide;
2-(4,6-Dimethoxybenzofuran-2-yl)-N-[(R)-1-(hydroxymethyl)-2-(1H-indol-3-yl)ethyl]-6-methoxyquinoline-4-carboxamide;
N-[(R)-1-(Hydroxymethyl)-2-(1H-indol-3-yl)ethyl]-6-methoxy-2-(5-methoxybenzofuran-2-yl)quinoline-4-carboxamide;
2-(7-Ethoxybenzofuran-2-yl)-N-[(R)-1-(hydroxymethyl)-2-(1H-indol-3-yl)ethyl]-6-methoxyquinoline-4-carboxamide;
N-[(R)-1-(Hydroxymethyl)-2-(1H-indol-3-yl)ethyl]-6-methoxy-2-(6-methoxybenzofuran-2-yl)quinoline-4-carboxamide;
2-(7-Fluorbenzofuran-2-yl)-N-[(R)-1-(hydroxymethyl)-2-(1H-indol-3-yl)ethyl]-6-methoxyquinoline-4-carboxamide;
2-(4-Fluorbenzofuran-2-yl)-N-[(R)-1-(hydroxymethyl)-2-(1H-indol-3-yl)ethyl]-6-methoxyquinoline-4-carboxamide;
N-[(R)-1-(Hydroxymethyl)-2-(1H-indol-3-yl)ethyl]-6-methoxy-2-(5-methylbenzofuran-2-yl)quinoline-4-carboxamide;
N-[(R)-1-(Hydroxymethyl)-2-(1H-indol-3-yl)ethyl]-6-methoxy-2-(7-methylbenzofuran-2-yl)quinoline-4-carboxamide;
N-[(R)-1-(Hydroxymethyl)-2-(1H-indol-3-yl)ethyl]-6-methoxy-2-(4-methoxybenzofuran-2-yl)quinoline-4-carboxamide;
N-[(R)-1-(Hydroxymethyl)-2-(1H-indol-3-yl)ethyl]-6-methoxy-2-[5-(trifluoromethoxy)benzofuran-2-yl]quinoline-4-carboxamide;
4-Ethoxybiphenyl-3-carboxylic acid [(R)-1-hydroxymethyl-2-(1H-indol-3-yl)ethyl]amide;
4-Ethoxy-3′-fluoro-4′-propoxybiphenyl-3-carboxylic acid [(R)-2-hydroxy-1-(1H-indol-3-ylmethyl)ethyl]amide;
2-Ethoxy-N-[(R)-1-hydroxymethyl-2-(1H-indol-3-yl)ethyl]-5-(6-methoxypyridin-3-yl)-benzamide;
4-Ethoxy-2′-fluoro-3′-methoxybiphenyl-3-carboxylic acid [(R)-2-hydroxy-1-(1H-indol-3-ylmethyl)ethyl]amide;
4′-Acetylamino-4-ethoxybiphenyl-3-carboxylic acid [(R)-1-hydroxymethyl-2-(1H-indol-3-yl)ethyl]amide;
2-Ethoxy-N-[(R)-1-hydroxymethyl-2-(1H-indol-3-yl)ethyl]-5-(2-methoxypyrimidin-5-yl)-benzamide;
4-Ethoxy-5′-fluoro-3′-methoxybiphenyl-3-carboxylic acid [(R)-2-hydroxy-1-(1H-indol-3-ylmethyl)ethyl]amide;
4-Ethoxy-3′,4′-difluoro-5′-methoxybiphenyl-3-carboxylic acid [(R)-2-hydroxy-1-(1H-indol-3-ylmethyl)ethyl]amide;
4-Ethoxy-4′-fluoro-3′-methoxybiphenyl-3-carboxylic acid [(R)-2-hydroxy-1-(1H-indol-3-ylmethyl)ethyl]amide;
3′,5′-Dimethoxy-4-propoxybiphenyl-3-carboxylic acid [(R)-1-hydroxymethyl-2-(1H-indol-3-yl)ethyl]amide;
4-Ethoxy-3′-hydroxymethylbiphenyl-3-carboxylic acid [(R)-1-hydroxymethyl-2-(1H-indol-3-yl)ethyl]amide;
4-Ethoxy-3′-methylsulphanylbiphenyl-3-carboxylic acid [(R)-2-hydroxy-1-(1H-indol-3-ylmethyl)ethyl]amide;
3′-Cyano-4-ethoxybiphenyl-3-carboxylic acid [(R)-1-hydroxymethyl-2-(1H-indol-3-yl)ethyl]amide;
2-Ethoxy-5-(6-fluoro-5-methylpyridin-3-yl)-N-[(R)-2-hydroxy-1-(1H-indol-3-ylmethyl)ethyl]benzamide;
4-Ethoxy-4′-trifluoromethoxybiphenyl-3-carboxylic acid [(R)-2-hydroxy-1-(1H-indol-3-ylmethyl)ethyl]amide;
5-Benzo[b]thiophene-3-yl-2-ethoxy-N-[(R)-2-hydroxy-1-(1H-indol-3-ylmethyl)ethyl]benzamide;
4-Ethoxy-2′-trifluoromethylbiphenyl-3-carboxylic acid [(R)-2-hydroxy-1-(1H-indol-3-ylmethyl)ethyl]amide;
4-Ethoxy-2′-trifluoromethoxybiphenyl-3-carboxylic acid [(R)-2-hydroxy-1-(1H-indol-3-ylmethyl)ethyl]amide;
4-Ethoxy-3′-trifluoromethoxybiphenyl-3-carboxylic acid [(R)-2-hydroxy-1-(1H-indol-3-ylmethyl)ethyl]amide;
4-Ethoxy-3′-fluorobiphenyl-3-carboxylic acid [(R)-2-hydroxy-1-(1H-indol-3-ylmethyl)ethyl]amide;
4′-Chloro-4-ethoxybiphenyl-3-carboxylic acid [(R)-2-hydroxy-1-(1H-indol-3-ylmethyl)ethyl]amide;
4-Ethoxy-4′-methylsulphanylbiphenyl-3-carboxylic acid [(R)-2-hydroxy-1-(1H-indol-3-ylmethyl)ethyl]amide;
4-Ethoxy-3′-trifluoromethylbiphenyl-3-carboxylic acid [(R)-2-hydroxy-1-(1H-indol-3-ylmethyl)ethyl]amide;
3′-Chloro-4-ethoxybiphenyl-3-carboxylic acid [(R)-2-hydroxy-1-(1H-indol-3-ylmethyl)ethyl]amide;
4-Ethoxy-3′-methylbiphenyl-3-carboxylic acid [(R)-1-hydroxymethyl-2-(1H-indol-3-yl)ethyl]amide;
5-Benzofuran-2-yl-2-ethoxy-N-[(R)-1-hydroxymethyl-2-(1H-indol-3-yl)ethyl]benzamide;
4-Ethoxy-2′-methylsulphanylbiphenyl-3-carboxylic acid [(R)-2-hydroxy-1-(1H-indol-3-ylmethyl)ethyl]amide;
2-Ethoxy-N-[(R)-1-hydroxymethyl-2-(1H-indol-3-yl)ethyl]-5-(1H-indol-4-yl)benzamide;
2-Ethoxy-N-[(R)-2-hydroxy-1-(1H-indol-3-ylmethyl)ethyl]-5-(4-methylthiophen-2-yl)-benzamide;
3′-Acetylamino-4-ethoxybiphenyl-3-carboxylic acid [(R)-1-hydroxymethyl-2-(1H-indol-3-yl)ethyl]amide;
4-Ethoxy-2′-methylbiphenyl-3-carboxylic acid [(R)-1-hydroxymethyl-2-(1H-indol-3-yl)ethyl]amide;
2-Ethoxy-N-[(R)-1-hydroxymethyl-2-(1H-indol-3-yl)ethyl]-5-(5-methylfuran-2-yl)-benzamide;
3′-Chloro-4-ethoxy-4′-methylbiphenyl-3-carboxylic acid [(R)-2-hydroxy-1-(1H-indol-3-ylmethyl)ethyl]amide;
5-(2-Chloro-6-methylpyridin-3-yl)-2-ethoxy-N-[(R)-2-hydroxy-1-(1H-indol-3-ylmethyl)ethyl]benzamide;
4-Ethoxy-4′-fluorobiphenyl-3-carboxylic acid [(R)-2-hydroxy-1-(1H-indol-3-ylmethyl)ethyl]amide;
2-Ethoxy-N-[(R)-1-hydroxymethyl-2-(1H-indol-3-yl)ethyl]-5-naphthalen-1-yl-benzamide;
5-Benzo[b]thiophene-2-yl-2-ethoxy-N-[(R)-2-hydroxy-1-(1H-indol-3-ylmethyl)ethyl]benzamide;
4-Ethoxy-4′-methylbiphenyl-3-carboxylic acid [(R)-1-hydroxymethyl-2-(1H-indol-3-yl)ethyl]amide;
2-Ethoxy-N-[(R)-2-hydroxy-1-(1H-indol-3-ylmethyl)ethyl]-5-thiophen-3-yl-benzamide;
4-Ethoxy-4′-methoxybiphenyl-3-carboxylic acid [(R)-1-hydroxymethyl-2-(1H-indol-3-yl)ethyl]amide;
2′,4′-Dichloro-4-ethoxybiphenyl-3-carboxylic acid [(R)-2-hydroxy-1-(1H-indol-3-ylmethyl)ethyl]amide;
4′-Methoxy-4-propoxybiphenyl-3-carboxylic acid [(R)-1-hydroxymethyl-2-(1H-indol-3-yl)ethyl]amide;
4-Propoxybiphenyl-3-carboxylic acid [(R)-1-hydroxymethyl-2-(1H-indol-3-yl)ethyl]amide;
5-Benzofuran-2-yl-N-[(R)-1-hydroxymethyl-2-(1H-indol-3-yl)ethyl]-2-propoxybenzamide;
3′-Chloro-4-propoxybiphenyl-3-carboxylic acid [(R)-2-hydroxy-1-(1H-indol-3-ylmethyl)ethyl]amide;
5-Benzo[b]thiophene-2-yl-N-[(R)-2-hydroxy-1-(1H-indol-3-ylmethyl)ethyl]-2-propoxybenzamide;
3′-Fluoro-4′-methyl-4-propoxybiphenyl-3-carboxylic acid [(R)-2-hydroxy-1-(1H-indol-3-ylmethyl)ethyl]amide;
4-Propoxy-3′-trifluoromethylbiphenyl-3-carboxylic acid [(R)-2-hydroxy-1-(1H-indol-3-ylmethyl)ethyl]amide;
2′-Fluoro-5′-methoxy-4-propoxybiphenyl-3-carboxylic acid [(R)-2-hydroxy-1-(1H-indol-3-ylmethyl)ethyl]amide;
4-Propoxy-3′,5′-bis-trifluoromethylbiphenyl-3-carboxylic acid [(R)-2-hydroxy-1-(1H-indol-3-ylmethyl)ethyl]amide;
4′-Chloro-4-propoxybiphenyl-3-carboxylic acid [(R)-2-hydroxy-1-(1H-indol-3-ylmethyl)ethyl]amide;
5-Benzo[b]thiophen-3-yl-N-[(R)-2-hydroxy-1-(1H-indol-3-ylmethyl)ethyl]-2-propoxybenzamide;
4-Propoxy-4′-trifluoromethylbiphenyl-3-carboxylic acid [(R)-2-hydroxy-1-(1H-indol-3-ylmethyl)ethyl]amide;
3′-Hydroxy-4-propoxybiphenyl-3-carboxylic acid [(R)-1-hydroxymethyl-2-(1H-indol-3-yl)ethyl]amide;
N-[(R)-1-Hydroxymethyl-2-(1H-indol-3-yl)ethyl]-2-propoxy-5-quinoline-6-yl-benzamide;
5-(6-Fluoro-5-methylpyridin-3-yl)-N-[(R)-2-hydroxy-1-(1H-indol-3-ylmethyl)ethyl]-2-propoxybenzamide;
N-[(R)-1-Hydroxymethyl-2-(1H-indol-3-yl)ethyl]-5-(6-methoxypyridin-3-yl)-2-propoxybenzamide;
3′-Chloro-4′-methyl-4-propoxybiphenyl-3-carboxylic acid [(R)-2-hydroxy-1-(1H-indol-3-ylmethyl)ethyl]amide;
N-[(R)-1-Hydroxymethyl-2-(1H-indol-3-yl)ethyl]-2-propoxy-5-pyridin-4-yl-benzamide;
3′-Chloro-4′-fluoro-4-propoxybiphenyl-3-carboxylic acid [(R)-2-hydroxy-1-(1H-indol-3-ylmethyl)ethyl]amide;
3′-Acetylamino-4-propoxybiphenyl-3-carboxylic acid [(R)-1-hydroxymethyl-2-(1H-indol-3-yl)ethyl]amide;
3′,4′-Difluoro-4-propoxybiphenyl-3-carboxylic acid [(R)-2-hydroxy-1-(1H-indol-3-ylmethyl)ethyl]amide;
3′,5′-Difluoro-4-propoxybiphenyl-3-carboxylic acid [(R)-2-hydroxy-1-(1H-indol-3-ylmethyl)ethyl]amide;
3′-Cyano-4-propoxybiphenyl-3-carboxylic acid [(R)-1-hydroxymethyl-2-(1H-indol-3-yl)ethyl]amide;
5-(2,4-Dimethoxypyrimidin-5-yl)-N-[(R)-1-hydroxymethyl-2-(1H-indol-3-yl)ethyl]-2-propoxybenzamide;
2′,3′-Difluoro-4-propoxybiphenyl-3-carboxylic acid [(R)-2-hydroxy-1-(1H-indol-3-ylmethyl)ethyl]amide;
2′,5′-Difluoro-4-propoxybiphenyl-3-carboxylic acid [(R)-2-hydroxy-1-(1H-indol-3-ylmethyl)ethyl]amide;
5-[(E)-2-(4-Fluorophenyl)vinyl]-N-[(R)-2-hydroxy-1-(1H-indol-3-ylmethyl)ethyl]-2-propoxybenzamide;
5-(5-Cyanothiophen-2-yl)-N-[(R)-2-hydroxy-1-(1H-indol-3-ylmethyl)ethyl]-2-propoxybenzamide;
2′-Fluoro-3′-methoxy-4-propoxybiphenyl-3-carboxylic acid [(R)-2-hydroxy-1-(1H-indol-3-ylmethyl)ethyl]amide;
N-[(R)-1-Hydroxymethyl-2-(1H-indol-3-yl)ethyl]-5-(2-methoxypyrimidin-5-yl)-2-propoxybenzamide;
4′-Chloro-2′,6′-difluoro-4-propoxybiphenyl-3-carboxylic acid [(R)-2-hydroxy-1-(1H-indol-3-ylmethyl)ethyl]amide;
3′,5′-Dimethyl-4-propoxybiphenyl-3-carboxylic acid [(R)-1-hydroxymethyl-2-(1H-indol-3-yl)ethyl]amide;
N-[(R)-1-Hydroxymethyl-2-(1H-indol-3-yl)ethyl]-2-propoxy-5-quinolin-3-yl-benzamide;
4′-Acetylamino-4-propoxybiphenyl-3-carboxylic acid [(R)-1-hydroxymethyl-2-(1H-indol-3-yl)ethyl]amide;
4-Propoxy-3′-(2,2,2-trifluoroethoxy)biphenyl-3-carboxylic acid [(R)-2-hydroxy-1-(1H-indol-3-ylmethyl)ethyl]amide;
3′-Ethoxy-5′-fluoro-4-propoxybiphenyl-3-carboxylic acid [(R)-2-hydroxy-1-(1H-indol-3-ylmethyl)ethyl]amide;
5′-Ethoxy-2′-fluoro-4-propoxybiphenyl-3-carboxylic acid [(R)-2-hydroxy-1-(1H-indol-3-ylmethyl)ethyl]amide;
3′-Ethoxy-4-propoxybiphenyl-3-carboxylic acid [(R)-1-hydroxymethyl-2-(1H-indol-3-yl)ethyl]amide;
4-Propoxybiphenyl-3-carboxylic acid [(R)-1-hydroxymethyl-2-(1-methyl-1H-indol-3-yl)ethyl]amide;
5-Benzofuran-2-yl-N-[(R)-1-hydroxymethyl-2-(1-methyl-1H-indol-3-yl)ethyl]-2-propoxybenzamide;
5-Benzo[b]thiophen-2-yl-N-[(R)-2-hydroxy-1-(1-methyl-1H-indol-3-ylmethyl)ethyl]-2-propoxybenzamide;
2′-Fluoro-4′-methyl-4-propoxybiphenyl-3-carboxylic acid [(R)-2-hydroxy-1-(1-methyl-1H-indol-3-ylmethyl)ethyl]amide;
4′-Fluoro-4-propoxybiphenyl-3-carboxylic acid [(R)-2-hydroxy-1-(1-methyl-1H-indol-3-ylmethyl)ethyl]amide;
2′-Fluoro-5′-methoxy-4-propoxybiphenyl-3-carboxylic acid [(R)-2-hydroxy-1-(1-methyl-1H-indol-3-ylmethyl)ethyl]amide;
3′-Fluoro-4-propoxybiphenyl-3-carboxylic acid [(R)-2-hydroxy-1-(1-methyl-1H-indol-3-ylmethyl)ethyl]amide;
N-[(R)-1-Hydroxymethyl-2-(1-methyl-1H-indol-3-yl)ethyl]-2-propoxy-5-pyridin-3-yl-benzamide;
5-Benzo[b]thiophen-3-yl-N-[(R)-2-hydroxy-1-(1-methyl-1H-indol-3-ylmethyl)ethyl]-2-propoxybenzamide;
3′-Cyano-4′-fluoro-4-propoxybiphenyl-3-carboxylic acid [(R)-2-hydroxy-1-(1-methyl-1H-indol-3-ylmethyl)ethyl]amide;
N-[(R)-1-Hydroxymethyl-2-(1-methyl-1H-indol-3-yl)ethyl]-5-(6-methoxypyridin-3-yl)-2-propoxybenzamide;
3′-Acetylamino-4-propoxybiphenyl-3-carboxylic acid [(R)-1-hydroxymethyl-2-(1-methyl-1H-indol-3-yl)ethyl]amide;
3′,4′-Difluoro-4-propoxybiphenyl-3-carboxylic acid [(R)-2-hydroxy-1-(1-methyl-1H-indol-3-ylmethyl)ethyl]amide;
3′,5′-Difluoro-4-propoxybiphenyl-3-carboxylic acid [(R)-2-hydroxy-1-(1-methyl-1H-indol-3-ylmethyl)ethyl]amide;
5-(2,4-Dimethoxypyrimidin-5-yl)-N-[(R)-1-hydroxymethyl-2-(1-methyl-1H-indol-3-yl)ethyl]-2-propoxybenzamide;
2′,5′-Difluoro-4-propoxybiphenyl-3-carboxylic acid [(R)-2-hydroxy-1-(1-methyl-1H-indol-3-ylmethyl)ethyl]amide;
5-[(E)-2-(4-Fluorophenyl)vinyl]-N-[(R)-2-hydroxy-1-(1-methyl-1H-indol-3-ylmethyl)ethyl]-2-propoxybenzamide;
5-(5-Cyanothiophen-2-yl)-N-[(R)-2-hydroxy-1-(1-methyl-1H-indol-3-ylmethyl)ethyl]-2-propoxybenzamide;
N-[(R)-1-Hydroxymethyl-2-(1-methyl-1H-indol-3-yl)ethyl]-5-(2-methoxypyrimidin-5-yl)-2-propoxybenzamide;
N-[(R)-1-Hydroxymethyl-2-(1-methyl-1H-indol-3-yl)ethyl]-2-propoxy-5-quinoline-3-yl-benzamide;
5′-Fluoro-3′-methoxy-4-propoxybiphenyl-3-carboxylic acid [(R)-2-hydroxy-1-(1-methyl-1H-indol-3-ylmethyl)ethyl]amide;
4-Propoxy-3′-(2,2,2-trifluoroethoxy)biphenyl-3-carboxylic acid [(R)-2-hydroxy-1-(1-methyl-1H-indol-3-ylmethyl)ethyl]amide;
5′-Ethoxy-2′-fluoro-4-propoxybiphenyl-3-carboxylic acid [(R)-2-hydroxy-1-(1-methyl-1H-indol-3-ylmethyl)ethyl]amide;
4′-Methoxy-4-propoxybiphenyl-3-carboxylic acid [2-(5-fluoro-1H-indol-3-yl)-1-hydroxymethylethyl]amide;
5-Benzofuran-2-yl-N-[2-(5-fluoro-1H-indol-3-yl)-1-hydroxymethylethyl]-2-propoxybenzamide;
3′-Methyl-4-propoxybiphenyl-3-carboxylic acid [2-(5-fluoro-1H-indol-3-yl)-1-hydroxymethylethyl]amide;
5-Benzo[b]thiophen-2-yl-N-[2-(5-fluoro-1H-indol-3-yl)-1-hydroxymethylethyl]-2-propoxybenzamide;
2′-Fluoro-5′-methoxy-4-propoxybiphenyl-3-carboxylic acid [1-(5-fluoro-1H-indol-3-ylmethyl)-2-hydroxyethyl]amide;
4-Propoxy-3′,5′-bis-trifluoromethylbiphenyl-3-carboxylic acid [1-(5-fluoro-1H-indol-3-ylmethyl)-2-hydroxyethyl]amide;
N-[2-(5-Fluoro-1H-indol-3-yl)-1-hydroxymethylethyl]-2-propoxy-5-pyridin-3-yl-benzamide;
5-Benzo[b]thiophen-3-yl-N-[2-(5-fluoro-1H-indol-3-yl)-1-hydroxymethylethyl]-2-propoxybenzamide;
3′-Cyano-4′-fluoro-4-propoxybiphenyl-3-carboxylic acid [1-(5-fluoro-1H-indol-3-ylmethyl)-2-hydroxyethyl]amide;
N-[1-(5-Fluoro-1H-indol-3-ylmethyl)-2-hydroxyethyl]-5-(6-fluoro-5-methylpyridin-3-yl)-2-propoxybenzamide;
N-[2-(5-Fluoro-1H-indol-3-yl)-1-hydroxymethylethyl]-5-(6-methoxypyridin-3-yl)-2-propoxybenzamide;
3′-Chloro-4′-fluoro-4-propoxybiphenyl-3-carboxylic acid [1-(5-fluoro-1H-indol-3-ylmethyl)-2-hydroxyethyl]amide;
3′-Acetylamino-4-propoxybiphenyl-3-carboxylic acid [2-(5-fluoro-1H-indol-3-yl)-1-hydroxymethylethyl]amide;
3′,4′-Difluoro-4-propoxybiphenyl-3-carboxylic acid [1-(5-fluoro-1H-indol-3-ylmethyl)-2-hydroxyethyl]amide;
3′,5′-Difluoro-4-propoxybiphenyl-3-carboxylic acid [2-(5-fluoro-1H-indol-3-yl)-1-hydroxymethylethyl]amide;
2′,5′-Difluoro-4-propoxybiphenyl-3-carboxylic acid [1-(5-fluoro-1H-indol-3-ylmethyl)-2-hydroxyethyl]amide;
N-[2-(5-Fluoro-1H-indol-3-yl)-1-hydroxymethylethyl]-5-[(E)-2-(4-fluoro-phenyl)vinyl]-2-propoxybenzamide;
5-(5-Cyanothiophen-2-yl)-N-[2-(5-fluoro-1H-indol-3-yl)-1-hydroxymethylethyl]-2-propoxybenzamide;
2′-Fluoro-3′-methoxy-4-propoxybiphenyl-3-carboxylic acid [1-(5-fluoro-1H-indol-3-ylmethyl)-2-hydroxyethyl]amide;
N-[2-(5-Fluoro-1H-indol-3-yl)-1-hydroxymethylethyl]-5-(2-methoxypyrimidin-5-yl)-2-propoxybenzamide;
N-[2-(5-Fluoro-1H-indol-3-yl)-1-hydroxymethylethyl]-2-propoxy-5-quinolin-3-yl-benzamide;
4-Propoxy-3′-(2,2,2-trifluoroethoxy)biphenyl-3-carboxylic acid [1-(5-fluoro-1H-indol-3-ylmethyl)-2-hydroxyethyl]amide;
5′-Ethoxy-2′-fluoro-4-propoxybiphenyl-3-carboxylic acid [1-(5-fluoro-1H-indol-3-ylmethyl)-2-hydroxyethyl]amide;
3′-Methoxy-4-propoxybiphenyl-3-carboxylic acid [(R)-1-hydroxymethyl-2-(1-methyl-1H-indol-3-yl)ethyl]amide;
3′-Chloro-4-propoxybiphenyl-3-carboxylic acid [(R)-2-hydroxy-1-(1-methyl-1H-indol-3-ylmethyl)ethyl]amide;
4-Propoxy-3′,5′-bis-trifluoromethylbiphenyl-3-carboxylic acid [(R)-2-hydroxy-1-(1-methyl-1H-indol-3-ylmethyl)ethyl]amide;
3′,4′,5′-Trifluoro-4-propoxybiphenyl-3-carboxylic acid [(R)-2-hydroxy-1-(1-methyl-1H-indol-3-ylmethyl)ethyl]amide;
4-Propoxy-4′-trifluoromethoxybiphenyl-3-carboxylic acid [(R)-2-hydroxy-1-(1-methyl-1H-indol-3-ylmethyl)ethyl]amide;
4-Propoxy-4′-trifluoromethylbiphenyl-3-carboxylic acid [(R)-2-hydroxy-1-(1-methyl-1H-indol-3-ylmethyl)ethyl]amide;
5-(6-Fluoro-5-methylpyridin-3-yl)-N-[(R)-2-hydroxy-1-(1-methyl-1H-indol-3-ylmethyl)ethyl]-2-propoxybenzamide;
5-(3,5-Dimethylisoxazol-4-yl)-N-[(R)-1-hydroxymethyl-2-(1-methyl-1H-indol-3-yl)ethyl]-2-propoxybenzamide;
3′-Chloro-4′-fluoro-4-propoxybiphenyl-3-carboxylic acid [(R)-2-hydroxy-1-(1-methyl-1H-indol-3-ylmethyl)ethyl]amide;
3′-Cyano-4-propoxybiphenyl-3-carboxylic acid [(R)-1-hydroxymethyl-2-(1-methyl-1H-indol-3-yl)ethyl]amide;
2′,3′-Difluoro-4-propoxybiphenyl-3-carboxylic acid [(R)-2-hydroxy-1-(1-methyl-1H-indol-3-ylmethyl)ethyl]amide;
3′,5′-Dimethyl-4-propoxybiphenyl-3-carboxylic acid [(R)-1-hydroxymethyl-2-(1-methyl-1H-indol-3-yl)ethyl]amide;
3′-Ethoxy-5′-fluoro-4-propoxybiphenyl-3-carboxylic acid [(R)-2-hydroxy-1-(1-methyl-1H-indol-3-ylmethyl)ethyl]amide;
5′-Fluoro-3′-hydroxy-4-propoxybiphenyl-3-carboxylic acid [(R)-2-hydroxy-1-(1-methyl-1H-indol-3-ylmethyl)ethyl]amide;
4,3′-Dipropoxybiphenyl-3-carboxylic acid [(R)-1-hydroxymethyl-2-(1-methyl-1H-indol-3-yl)ethyl]amide;
3′-Chloro-4-propoxybiphenyl-3-carboxylic acid [2-(5-fluoro-1H-indol-3-yl)-1-hydroxymethylethyl]amide;
3′-Fluoro-4′-methyl-4-propoxybiphenyl-3-carboxylic acid [1-(5-fluoro-1H-indol-3-ylmethyl)-2-hydroxyethyl]amide;
2′-Fluoro-4′-methyl-4-propoxybiphenyl-3-carboxylic acid [1-(5-fluoro-1H-indol-3-ylmethyl)-2-hydroxyethyl]amide;
4-Propoxy-3′-trifluoromethylbiphenyl-3-carboxylic acid [1-(5-fluoro-1H-indol-3-ylmethyl)-2-hydroxyethyl]amide;
3′-Isopropyl-4-propoxybiphenyl-3-carboxylic acid [2-(5-fluoro-1H-indol-3-yl)-1-hydroxymethylethyl]amide;
3′-Methylsulphanyl-4-propoxybiphenyl-3-carboxylic acid [2-(5-fluoro-1H-indol-3-yl)-1-hydroxymethylethyl]amide;
4-Propoxy-4′-trifluoromethoxybiphenyl-3-carboxylic acid [1-(5-fluoro-1H-indol-3-ylmethyl)-2-hydroxyethyl]amide;
N-[2-(5-Fluoro-1H-indol-3-yl)-1-hydroxymethylethyl]-2-propoxy-5-quinolin-6-yl-benzamide;
3′-Chloro-4′-methyl-4-propoxybiphenyl-3-carboxylic acid [2-(5-fluoro-1H-indol-3-yl)-1-hydroxymethylethyl]amide;
5-(3,5-Dimethylisoxazol-4-yl)-N-[2-(5-fluoro-1H-indol-3-yl)-1-hydroxymethylethyl]-2-propoxybenzamide;
2′,3′-Difluoro-4-propoxybiphenyl-3-carboxylic acid [1-(5-fluoro-1H-indol-3-ylmethyl)-2-hydroxyethyl]amide;
3′,5′-Dimethyl-4-propoxybiphenyl-3-carboxylic acid [2-(5-fluoro-1H-indol-3-yl)-1-hydroxymethylethyl]amide;
5′-Ethoxy-3′-fluoro-4-propoxybiphenyl-3-carboxylic acid [2-(5-fluoro-1H-indol-3-yl)-1-hydroxymethylethyl]amide;
3′-Fluoro-5′-hydroxy-4-propoxybiphenyl-3-carboxylic acid [2-(5-fluoro-1H-indol-3-yl)-1-hydroxymethylethyl]amide;
4,3′-Dipropoxybiphenyl-3-carboxylic acid [2-(5-fluoro-1H-indol-3-yl)-1-hydroxymethylethyl]amide;
3′-Ethoxy-4-propoxybiphenyl-3-carboxylic acid [2-(5-fluoro-1H-indol-3-yl)-1-hydroxymethylethyl]amide;
4′-Hydroxymethyl-4-propoxybiphenyl-3-carboxylic acid [(R)-1-hydroxymethyl-2-(1H-indol-3-yl)ethyl]amide;
3′-Hydroxymethyl-4-propoxybiphenyl-3-carboxylic acid [(R)-1-hydroxymethyl-2-(1H-indol-3-yl)ethyl]amide;
4-Propoxybiphenyl-3,4′-dicarboxylic acid 3-{[(R)-1-hydroxymethyl-2-(1H-indol-3-yl)ethyl]amide}4′-methylamide;
N-[(R)-2-Hydroxy-1-(1H-indol-3-ylmethyl)ethyl]-5-(5-hydroxymethylthiophen-2-yl)-2-propoxybenzamide;
5′-Fluoro-4-propoxybiphenyl-3,3′-dicarboxylic acid 3-{[(R)-2-hydroxy-1-(1H-indol-3-ylmethyl)ethyl]amide}3′-methylamide;
3′-Chloro-4-propoxybiphenyl-3,4′-dicarboxylic acid 3-{[(R)-2-hydroxy-1-(1H-indol-3-ylmethyl)ethyl]amide}4′-methylamide;
3′-Hydroxymethyl-4-propoxybiphenyl-3-carboxylic acid [(R)-1-hydroxymethyl-2-(1-methyl-1H-indol-3-yl)ethyl]amide;
3′-Hydroxymethyl-4-propoxybiphenyl-3-carboxylic acid [2-(5-fluoro-1H-indol-3-yl)-1-hydroxymethylethyl]amide;
3′-Chloro-4-propoxybiphenyl-3,4′-dicarboxylic acid 3-{([2-(5-fluoro-1H-indol-3-yl)-1-hydroxymethylethyl]amide}4′-methylamide;
N-[(R)-2-Hydroxy-1-(1-methyl-1H-indol-3-ylmethyl)ethyl]-5-(5-hydroxymethylthiophen-2-yl)-2-propoxybenzamide;
3′-Chloro-4-propoxybiphenyl-3,4′-dicarboxylic acid 3-{[(R)-2-hydroxy-1-(1-methyl-1H-indol-3-ylmethyl)ethyl]amide}4′-methylamide;
4′-Hydroxymethyl-4-propoxybiphenyl-3-carboxylic acid [2-(5-fluoro-1H-indol-3-yl)-1-hydroxymethylethyl]amide;
4-Propoxybiphenyl-3,4′-dicarboxylic acid 3-{[2-(5-fluoro-1H-indol-3-yl)-1-hydroxymethylethyl]amide}4′-methylamide;
5′-Fluoro-4-propoxybiphenyl-3,3′-dicarboxylic acid 3-{[2-(5-fluoro-1H-indol-3-yl)-1-hydroxymethylethyl]amide}3′-methylamide;
4-Ethoxy-3′-fluoro-N-[(R)-1-(hydroxymethyl)-2-(1H-indol-3-yl)ethyl]-4′-methoxy[1,1′-biphenyl]-3-carboxamide;
4-Ethoxy-N-[(R)-1-(hydroxymethyl)-2-(1H-indol-3-yl)ethyl]-3′-methoxy[, 1′-biphenyl]-3-carboxamide;
4-Ethoxy-N-[(R)-1-(hydroxymethyl)-2-(1H-indol-3-yl)ethyl]-N′-methyl[1,1′-biphenyl]-3,3′-dicarboxamide;
4-Ethoxy-N-[(R)-1-(hydroxymethyl)-2-(1H-indol-3-yl)ethyl]-3′,4′,5′-trimethoxy[1,1′-biphenyl]-3-carboxamide;
4-Ethoxy-N-[(R)-1-(hydroxymethyl)-2-(1H-indol-3-yl)ethyl]-3′,4′-dimethoxy[1,1′-biphenyl]-3-carboxamide;
4-Ethoxy-N-[(R)-1-(hydroxymethyl)-2-(1H-indol-3-yl)ethyl]-3′-(1-methylethyl)[1,1′-biphenyl]-3-carboxamide;
N-[(R)-1-(Hydroxymethyl)-2-(1H-indol-3-yl)ethyl]-3′,4′,5′-trimethoxy-4-propoxy[1,1′-biphenyl]-3-carboxamide;
N-[(R)-1-(Hydroxymethyl)-2-(1H-indol-3-yl)ethyl]-3′,4′-dimethoxy-4-propoxy[1,1′-biphenyl]-3-carboxamide;
N-[(R)-1-(Hydroxymethyl)-2-(1H-indol-3-yl)ethyl]-3′-methoxy-4-propoxy[1,1′-biphenyl]-3-carboxamide;
N-[(R)-1-(Hydroxymethyl)-2-(1H-indol-3-yl)ethyl]-N′-methyl-4-propoxy[1,1′-biphenyl]-3,3′-dicarboxamide;
4,3′,4′,5′-Tetramethoxybiphenyl-3-carboxylic acid [(R)-1-hydroxymethyl-2-(1H-indol-3-yl)ethyl]amide;
4,3′,4′-Trimethoxybiphenyl-3-carboxylic acid [(R)-1-hydroxymethyl-2-(1H-indol-3-yl)ethyl]amide;
3′-Fluoro-4,4′-dimethoxybiphenyl-3-carboxylic acid [(R)-2-hydroxy-1-(1H-indol-3-ylmethyl)ethyl]amide;
4,3′-Dimethoxybiphenyl-3-carboxylic acid [(R)-1-hydroxymethyl-2-(1H-indol-3-yl)ethyl]amide;
5-Benzo[1,3]dioxol-5-yl-N-[(R)-1-hydroxymethyl-2-(1H-indol-3-yl)ethyl]-2-methoxybenzamide;
3′,4′-Difluoro-4,5′-dimethoxybiphenyl-3-carboxylic acid [(R)-2-hydroxy-1-(1H-indol-3-ylmethyl)ethyl]amide;
4-Isopropoxy-3′-methoxybiphenyl-3-carboxylic acid [(R)-1-hydroxymethyl-2-(1H-indol-3-yl)ethyl]amide;
5-Benzo[1,3]dioxol-5-yl-N-[(R)-1-hydroxymethyl-2-(1H-indol-3-yl)ethyl]-2-isopropoxybenzamide;
4-Isopropoxy-3′,4′,5′-trimethoxybiphenyl-3-carboxylic acid [(R)-1-hydroxymethyl-2-(1H-indol-3-yl)ethyl]amide;
3′-Fluoro-4-isopropoxy-4′-methoxybiphenyl-3-carboxylic acid [(R)-2-hydroxy-1-(1H-indol-3-ylmethyl)ethyl]amide;
4-Isopropoxy-3′,4′-dimethoxybiphenyl-3-carboxylic acid [(R)-1-hydroxymethyl-2-(1H-indol-3-yl)ethyl]amide;
4-Isopropoxy-3′-methylbiphenyl-3-carboxylic acid [(R)-1-hydroxymethyl-2-(1H-indol-3-yl)ethyl]amide;
4′-Fluoro-4-isopropoxy-3′-methylbiphenyl-3-carboxylic acid [(R)-2-hydroxy-1-(1H-indol-3-ylmethyl)ethyl]amide;
3′,4′-Difluoro-4-isopropoxy-5′-methoxybiphenyl-3-carboxylic acid [(R)-2-hydroxy-1-(1H-indol-3-ylmethyl)ethyl]amide;
4,3′,4′,5′-Tetramethoxy-5-methylbiphenyl-3-carboxylic acid [(R)-1-hydroxymethyl-2-(1H-indol-3-yl)ethyl]amide;
4,3′,4′-Trimethoxy-5-methylbiphenyl-3-carboxylic acid [(R)-1-hydroxymethyl-2-(1H-indol-3-yl)ethyl]amide;
3′-Fluoro-4,4′-dimethoxy-5-methylbiphenyl-3-carboxylic acid [(R)-2-hydroxy-1-(1H-indol-3-ylmethyl)ethyl]amide;
5-Benzo[1,3]dioxol-5-yl-N-[(R)-1-hydroxymethyl-2-(1H-indol-3-yl)ethyl]-2-methoxy-3-methylbenzamide;
4,3′-Dimethoxy-5-methylbiphenyl-3-carboxylic acid [(R)-1-hydroxymethyl-2-(1H-indol-3-yl)ethyl]amide;
4-Methoxy-5,3′-dimethylbiphenyl-3-carboxylic acid [(R)-1-hydroxymethyl-2-(1H-indol-3-yl)ethyl]amide;
4′-Fluoro-4-methoxy-5,3′-dimethylbiphenyl-3-carboxylic acid [(R)-2-hydroxy-1-(1H-indol-3-ylmethyl)ethyl)amide;
3′,4′-Difluoro-4,5′-dimethoxy-5-methylbiphenyl-3-carboxylic acid [(R)-2-hydroxy-1-(1H-indol-3-ylmethyl)ethyl]amide;
3′-Hydroxy-4-isopropoxybiphenyl-3-carboxylic acid ((R)-1-hydroxymethyl-2-(1H-indol-3-yl)ethyl]amide;
3′,4′,5′-Trimethoxy-4-(3-methylbut-2-enyloxy)biphenyl-3-carboxylic acid [(R)-1-hydroxymethyl-2-(1H-indol-3-yl)ethyl]amide;
3′-Butoxy-4-ethoxybiphenyl-3-carboxylic acid [(R)-1-hydroxymethyl-2-(1H-indol-3-yl)ethyl]amide;
4-Ethoxy-3′-isopropoxybiphenyl-3-carboxylic acid [(R)-1-hydroxymethyl-2-(1H-indol-3-yl)ethyl]amide;
N-[(R)-1-Hydroxymethyl-2-(1H-indol-3-yl)ethyl]-5-(7-methoxybenzofuran-2-yl)-2-propoxybenzamide;
6-Methoxy-2-(3,4,5-trimethoxyphenyl)quinoline-4-carboxylic acid [(R)-2-(6-chloro-1H-indol-3-yl)-1-hydroxymethylethyl]amide;
6-Methoxy-2-(3,4,5-trimethoxyphenyl)quinoline-4-carboxylic acid [(R)-1-hydroxymethyl-2-(2-methyl-1H-indol-3-yl)ethyl]amide;
6-Methoxy-2-(3,4,5-trimethoxyphenyl)quinoline-4-carboxylic acid [1-hydroxymethyl-2-(6-methyl-1H-indol-3-yl)ethyl]amide;
N-[(R)-1-(Hydroxymethyl)-2-(1-ethyl)-1H-indol-3-yl)ethyl]-6-methoxy-2-(3-methoxyphenyl)quinoline-4-carboxamide;
N-[(R)-1-(Hydroxymethyl)-2-(1-propyl-1H-indol-3-yl)ethyl]-6-methoxy-2-(3,4,5-trimethoxyphenyl)quinoline-4-carboxamide;
N-[(R)-1-(Hydroxymethyl)-2-(1-ethyl)-1H-indol-3-yl)ethyl]-2-(3,5-difluoro-4-methoxyphenyl)-6-methoxyquinoline-4-carboxamide;
N-[(R)-1-(Hydroxymethyl)-2-(1-isopropyl-1H-indol-3-yl)ethyl]-6-methoxy-2-(3-methoxyphenyl)-quinoline-4-carboxamide;
N-[(R)-1-(Hydroxymethyl)-2-(1-isopropyl-1H-indol-3-yl)ethyl]-2-(3,5-difluoro-4-methoxyphenyl)-6-methoxyquinoline-4-carboxamide;
N-[(R)-1-(Hydroxymethyl)-2-(1-isopropyl-1H-indol-3-yl)ethyl]-6-methoxy-2-(3,4,5-trimethoxyphenyl)quinoline-4-carboxamide;
N-[(R)-1-(Hydroxymethyl)-2-(1-ethyl)-1H-indol-3-yl)ethyl]-2-(3-fluoro-4-methoxyphenyl)-6-trifluoromethoxyquinoline-4-carboxamide;
N-[(R)-1-(Hydroxymethyl)-2-(1-ethyl)-1H-indol-3-yl)ethyl]-2-(7-methoxybenzofuran-2-yl)-6-trifluoromethoxyquinoline-4-carboxamide;
N-[(R)-1-(Hydroxymethyl)-2-(1-isopropyl-1H-indol-3-yl)ethyl]-2-(3-fluoro-4-methoxyphenyl)-6-trifluoromethoxyquinoline-4-carboxamide;
N-[(R)-1-(Hydroxymethyl)-2-(1-isopropyl-1H-indol-3-yl)ethyl]-2-(7-methoxybenzofuran-2-yl)-6-trifluoromethoxyquinoline-4-carboxamide;
N-[(R)-1-(Hydroxymethyl)-2-(1-n-hexyl-1H-indol-3-yl)ethyl]-6-methoxy-2-(3,4,5-trimethoxyphenyl)quinoline-4-carboxamide;
N-[(R)-1-(Hydroxymethyl)-2-(1-ethyl)-1H-indol-3-yl)ethyl]-4-ethoxy-3′-methoxybiphenyl-3-carboxamide;
N-[(R)-1-(Hydroxymethyl)-2-(1-isopropyl)-1H-indol-3-yl)ethyl]-4-ethoxy-3′-methoxybiphenyl)-3-carboxamide;
N-[(R)-2-(1-Ethyl-1H-indol-3-yl)-1-(hydroxymethyl)ethyl]-3′-fluoro-4′-methoxy[1,1′-biphenyl]-3-carboxamide;
3′-Fluoro-N-[(R)-1-(hydroxymethyl)-2-(1-propyl-1H-indol-3-yl)ethyl]-4′-methoxy[1,1′-biphenyl]-3-carboxamide;
N-[(R)-2-(1-Butyl-1H-indol-3-yl)-1-(hydroxymethyl)ethyl]-3′-fluoro-4′-methoxy[1,1′-biphenyl]-3-carboxamide;
3′-Fluoro-N-[(R)-1-(hydroxymethyl)-2-[1-(3-methylbutyl)-1H-indol-3-yl]ethyl]-4′-methoxy[1,1′-biphenyl]-3-carboxamide;
3′-Fluoro-N-[(R)-1-(hydroxymethyl)-2-(1-pentyl-1H-indol-3-yl)ethyl]-4′-methoxy[1,1′-biphenyl]-3-carboxamide;
3′-Fluoro-N-[(R)-2-(1-hexyl-1H-indol-3-yl)-1-(hydroxymethyl)ethyl]-4′-methoxy[1,1′-biphenyl]-3-carboxamide;
4-Ethoxy-3′-methoxybiphenyl-3-carboxylic acid [2-(5,6-difluoro-1H-indol-3-yl)-1-hydroxymethylethyl]amide;
6-Methoxy-2-(3,4,5-trimethoxyphenyl)quinoline-4-carboxylic acid [2-(5,6-difluoro-1H-indol-3-yl)-1-hydroxymethylethyl]amide;
N-[(R)-1-(Hydroxymethyl)-2-(1-ethyl-5-fluoro-1H-indol-3-yl)ethyl]-6-methoxy-2-(3,4,5-trimethoxyphenyl)quinoline-4-carboxamide;
6-Methoxy-2-(3,4,5-trimethoxyphenyl)quinoline-4-carboxylic acid [2-(1-ethyl-5-fluoro-1H-indol-3-yl)-1-hydroxymethylethyl]amide;
6-(3,4,5-Trimethoxyphenyl)quinoline-8-carboxylic acid [(R)-1-hydroxymethyl-2-(1H-indol-3-yl)ethyl]amide;
3-(3,4,5-Trimethoxyphenyl)naphthalene-1-carboxylic acid [(R)-1-hydroxymethyl-2-(1H-indol-3-yl)ethyl]amide;
4-Methoxy-5-(3,4,5-trimethoxyphenyl)thiophene-3-carboxylic acid [(R)-2-hydroxy-1-(1H-indol-3-ylmethyl)ethyl]amide;
6-(3,4,5-Trimethoxyphenyl)-1H-benzoimidazole-4-carboxylic acid [(R)-2-hydroxy-1-(1H-indol-3-ylmethyl)ethyl]amide;
2-(3,4,5-Trimethoxyphenyl)thiazole-4-carboxylic acid [(R)-2-hydroxy-1-(1H-indol-3-ylmethyl)ethyl]amide;
5-(3,4,5-Trimethoxyphenyl)thiophene-2-carboxylic acid [(R)-2-hydroxy-1-(1H-indol-3-ylmethyl)ethyl]amide;
5-(3,4,5-Trimethoxyphenyl)benzo[b]thiophene-2carboxylic acid [(R)-2-hydroxy-1-(1H-indol-3-ylmethyl)ethyl]amide;
2-(3-Fluoro-4-methoxyphenyl)-N-[(R)-2-hydroxy-1-(1H-indol-3-ylmethyl)ethyl]-6-methylisonicotinamide;
2-(3-Fluoro-4-methoxyphenyl)-6-methylpyrimidine-4-carboxylic acid [(R)-2-hydroxy-1-(1H-indol-3-ylmethyl)ethyl]amide;
6-(4-Methoxyphenyl)pyrimidine-4-carboxylic acid [(R)-1-hydroxymethyl-2-(1H-indol-3-yl)ethyl]amide;
2-(3-Fluoro-4-methoxyphenyl)-6-methoxyquinazoline-4-carboxylic acid [(R)-2-hydroxy-1-(1H-indol-3-ylmethyl)ethyl]amide;
2-(3-Fluoro-4-methoxyphenyl)-6-iodoquinazoline-4-carboxylic acid [(R)-2-hydroxy-1-(1H-indol-3-ylmethyl)ethyl]amide;
2-(4-Methoxyphenyl)quinazoline-4-carboxylic acid [(R)-1-hydroxymethyl-2-(1H-indol-3-yl)ethyl]amide;
2-(3-Fluoro-4-methoxyphenyl)-6-methoxyquinazoline-4-carboxylic acid [(R)-1-(1-ethyl-1H-indol-3-ylmethyl)-2-hydroxyethyl]amide;
2-(3,4,5-Trimethoxyphenyl)quinoline-4-carboxylic acid [(R)-2-hydroxy-1-(1H-indol-3-ylmethyl)-2-methylpropyl]amide;
6-Methoxy-2-(3,4,5-trimethoxyphenyl)quinoline-4-carboxylic acid [(R)-2-hydroxy-1-(1H-indol-3-ylmethyl)-2-methylpropyl]amide;
6-(4-Hydroxybut-1-ynyl)-2-(3,4,5-trimethoxyphenyl)quinoline-4-carboxylic acid [(R)-1-hydroxymethyl-2-(1H-indol-3-yl)ethyl]amide;
6-(5-Hydroxypent-1-ynyl)-2-(3,4,5-trimethoxyphenyl)quinoline-4-carboxylic acid [(R)-1-hydroxymethyl-2-(1H-indol-3-yl)ethyl]amide;
6-(3-Hydroxyprop-1-ynyl)-2-(3,4,5-trimethoxyphenyl)quinoline-4-carboxylic acid [(R)-1-hydroxymethyl-2-(1H-indol-3-yl)ethyl]amide;
6-(3-Methoxyprop-1-ynyl)-2-(3,4,5-trimethoxyphenyl)quinoline-4-carboxylic acid [(R)-1-hydroxymethyl-2-(1H-indol-3-yl)ethyl]amide;
5-(4-Hydroxybut-1-ynyl)-3′,4′,5′-trimethoxybiphenyl-3-carboxylic acid [(R)-1-hydroxymethyl-2-(1H-indol-3-yl)ethyl]amide;
5-(3-Hydroxyprop-1-ynyl)-3′,4′,5′-trimethoxybiphenyl-3-carboxylic acid [(R)-1-hydroxymethyl-2-(1H-indol-3-yl)ethyl]amide;
5-(5-Hydroxypent-1-ynyl)-3′,4′,5′-trimethoxybiphenyl-3-carboxylic acid [(R)-1-hydroxymethyl-2-(1H-indol-3-yl)ethyl]amide;
3′,4′,5′-Trimethoxy-5-(3-methoxyprop-1-ynyl)biphenyl-3-carboxylic acid [(R)-1-hydroxymethyl-2-(1H-indol-3-yl)ethyl]amide;
3′,4′-Dimethoxy-5-(3-methoxyprop-1-ynyl)biphenyl-3-carboxylic acid [(R)-1-hydroxymethyl-2-(1H-indol-3-yl)ethyl]amide;
5-(3-Hydroxyprop-1-ynyl)-3′,4′-dimethoxybiphenyl-3-carboxylic acid [(R)-1-hydroxymethyl-2-(1H-indol-3-yl)ethyl]amide;
3′,4′,5′-Trimethoxy-5-(4-methoxyphenylethynyl)biphenyl-3-carboxylic acid [(R)-1-hydroxymethyl-2-(1H-indol-3-yl)ethyl]amide;
3′,4′,5′-Trimethoxy-5-((Z)-3-methoxypropenyl)biphenyl-3-carboxylic acid [(R)-1-hydroxymethyl-2-(1H-indol-3-yl)ethyl]amide;
5-((Z)-4-Hydroxybut-1-enyl)-3′,4′,5′-trimethoxybiphenyl-3-carboxylic acid [(R)-1-hydroxymethyl-2-(1H-indol-3-yl)ethyl]amide;
5-((Z)-3-Hydroxypropenyl)-3′,4′,5′-trimethoxybiphenyl-3-carboxylic acid [(R)-1-hydroxymethyl-2-(1H-indol-3-yl)ethyl]amide;
5-((Z)-5-Hydroxypent-1-enyl)-3′,4′,5′-trimethoxybiphenyl-3-carboxylic acid [(R)-1-hydroxymethyl-2-(1H-indol-3-yl)ethyl]amide;
6-(5-Hydroxypentyl)-2-(3,4,5-trimethoxyphenyl)quinoline-4-carboxylic acid [(R)-1-hydroxymethyl-2-(1H-indol-3-yl)ethyl]amide;
6-(4-Hydroxybutyl)-2-(3,4,5-trimethoxyphenyl)quinoline-4-carboxylic acid [(R)-1-hydroxymethyl-2-(1H-indol-3-yl)ethyl]amide;
6-(3-Hydroxypropyl)-2-(3,4,5-trimethoxyphenyl)quinoline-4-carboxylic acid [(R)-1-hydroxymethyl-2-(1H-indol-3-yl)ethyl]amide;
6-(3-Methoxypropyl)-2-(3,4,5-trimethoxyphenyl)quinoline-4-carboxylic acid [(R)-1-hydroxymethyl-2-(1H-indol-3-yl)ethyl]amide;
3′,4′,5′-Trimethoxy-4-(3-methoxypropyl)biphenyl-3-carboxylic acid [(R)-1-hydroxymethyl-2-(1H-indol-3-yl)ethyl]amide;
3′,4′,5′-Trimethoxy-5-(3-methoxypropyl)biphenyl-3-carboxylic acid [(R)-1-hydroxymethyl-2-(1H-indol-3-yl)ethyl]amide;
3′,4′-Dimethoxy-5-(3-methoxypropyl)biphenyl-3-carboxylic acid [(R)-1-hydroxymethyl-2-(1H-indol-3-yl)ethyl]amide;
5-(3-Hydroxypropyl)-3′,4′-dimethoxybiphenyl-3-carboxylic acid [(R)-1-hydroxymethyl-2-(1H-indol-3-yl)ethyl]amide;
5-(5-Hydroxypentyl)-3′,4′,5′-trimethoxybiphenyl-3-carboxylic acid [(R)-1-hydroxymethyl-2-(1H-indol-3-yl)ethyl]amide;
5-(3-Hydroxypropyl)-3′,4′,5′-trimethoxybiphenyl-3-carboxylic acid [(R)-1-hydroxymethyl-2-(1H-indol-3-yl)ethyl]amide;
5-(4-Hydroxybutyl)-3′,4′,5′-trimethoxybiphenyl-3-carboxylic acid [(R)-1-hydroxymethyl-2-(1H-indol-3-yl)ethyl]amide;
3′,4′,5′-Trimethoxy-5-[2-(4-methoxyphenyl)ethyl]-biphenyl-3-carboxylic acid [(R)-1-hydroxymethyl-2-(1H-indol-3-yl)ethyl]amide;
N-[(R)-2-[1-(2-Cyanethyl)-1H-indol-3-yl]-1-(hydroxymethyl)ethyl]-3′-fluoro-4′-methoxy[1,1′-biphenyl]-3-carboxamide;
3′-Fluoro-N-[(R)-2-(1-heptyl-1H-indol-3-yl)-1-(hydroxymethyl)ethyl]-4′-methoxy[1,1′-biphenyl]-3-carboxamide;
N-[(R)-2-[1-(4-Cyanobutyl)-1H-indol-3-yl]-1-(hydroxymethyl)ethyl]-3′-fluoro-4′-methoxy[1,1′-biphenyl]-3-carboxamide;
3′-Fluoro-N-[(R)-1-(hydroxymethyl)-2-[1-(3-phenoxypropyl)-1H-indol-3-yl]ethyl]-4′-methoxy[1,1′-biphenyl]-3-carboxamide;
3′-Fluoro-N-[(R)-1-(hydroxymethyl)-2-[1-(2-methoxyethyl)-1H-indol-3-yl]ethyl]-4′-methoxy[1,1′-biphenyl]-3-carboxamide;
N-[(R)-2-[1-(3-Cyanopropyl)-1H-indol-3-yl]-1-(hydroxymethyl)ethyl]-3′-fluoro-4′-methoxy[1,1′-biphenyl]-3-carboxamide;
4-Ethoxy-3′-methoxybiphenyl-3-carboxylic acid [(R)-2-(1-cyanomethyl-1H-indol-3-yl)-1-hydroxymethylethyl]amide;
6-Methoxy-2-(3,4,5-trimethoxyphenyl)quinoline-4-carboxylic acid [(R)-2-(1-cyanomethyl-1H-indol-3-yl)-1-hydroxymethylethyl]amide;
6-Methoxy-2-(3,4,5-trimethoxyphenyl)quinoline-4-carboxylic acid {(R)-2-[1-(4-cyanobutyl)-1H-indol-3-yl]-1-hydroxymethylethyl}-amide;
4-Hydroxy-3′,4′,5′-trimethoxybiphenyl-3-carboxylic acid [(R)-1-hydroxymethyl-2-(1H-indol-3-yl)ethyl]amide;
4-(3-Cyanopropoxy)-3′,4′,5′-trimethoxybiphenyl-3-carboxylic acid [(R)-1-hydroxymethyl-2-(1H-indol-3-yl)ethyl]amide;
4-Cyclopentyloxy-3′-fluoro-4′-methoxybiphenyl-3-carboxylic acid [(R)-2-hydroxy-1-(1H-indol-3-ylmethyl)ethyl]amide;
4-Cyclopentyloxy-3′-methylbiphenyl-3-carboxylic acid [(R)-1-hydroxymethyl-2-(1H-indol-3-yl)ethyl]amide;
3′-(1-Butyl-3-methylureido)-4-cyclopentyloxybiphenyl-3-carboxylic acid [(R)-1-hydroxymethyl-2-(1H-indol-3-yl)ethyl]amide;
4-Cyclopentyloxy-4′-fluoro-3′-methylbiphenyl-3-carboxylic acid [(R)-2-hydroxy-1-(1H-indol-3-ylmethyl)ethyl]amide;
4-Cyclopentyloxy-3′-methoxybiphenyl-3-carboxylic acid [(R)-1-hydroxymethyl-2-(1H-indol-3-yl)ethyl]amide;
4-Cyclopentyloxy-3′,4′-dimethoxybiphenyl-3-carboxylic acid [(R)-1-hydroxymethyl-2-(1H-indol-3-yl)ethyl]amide;
5-Benzo[1,3]dioxol-5-yl-2-cyclopentyloxy-N-[(R)-1-hydroxymethyl-2-(1H-indol-3-yl)ethyl]benzamide;
4-Cyclopentyloxy-3′,4′,5′-trimethoxybiphenyl-3-carboxylic acid [(R)-1-hydroxymethyl-2-(1H-indol-3-yl)ethyl]amide;
4-Cyclopentyloxy-3′,4′-difluoro-5′-methoxybiphenyl-3-carboxylic acid [(R)-2-hydroxy-1-(1H-indol-3-ylmethyl)ethyl]amide;
3′-[Butyl[(1,1-dimethylethoxy)carbonyl]amino]-4-ethoxy-N-[(R)-1-(hydroxymethyl)-2-(1H-indol-3-yl)ethyl][1,1′-biphenyl]-3-carboxamide;
3′-(Butylamino)-4-ethoxy-N-[(R)-1-(hydroxymethyl)-2-(1H-indol-3-yl)ethyl][1,1′-biphenyl]-3-carboxamide;
3′-[Butyl[(methylamino)carbonyl]amino]-4-ethoxy-N-[(R)-1-(hydroxymethyl)-2-(1H-indol-3-yl)ethyl][1,1′-biphenyl]-3-carboxamide;
3′-[Butyl[(1,1-dimethylethoxy)carbonyl]amino]-N-[(R)-1-(hydroxymethyl)-2-(1H-indol-3-yl)ethyl]-4-propoxy[1,1′-biphenyl]-3-carboxamide;
3′-(1-Butyl-3-methylureido)-4-methoxybiphenyl-3-carboxylic acid [(R)-1-hydroxymethyl-2-(1H-indol-3-yl)ethyl]amide;
3′-(1-Butyl-3-methylureido)-4-methoxy-5-methylbiphenyl-3-carboxylic acid [(R)-1-hydroxymethyl-2-(1H-indol-3-yl)ethyl]amide;
3′-(1-Butyl-3-methylureido)-4-isopropoxybiphenyl-3-carboxylic acid [(R)-1-hydroxymethyl-2-(1H-indol-3-yl)ethyl]amide;
3′-(2-Dimethylaminoethoxy)-4-ethoxybiphenyl-3-carboxylic acid [(R)-1-hydroxymethyl-2-(1H-indol-3-yl)ethyl]amide;
4′-Ethoxy-3′-[(R)-1-hydroxymethyl-2-(1H-indol-3-yl)ethylcarbamoyl]biphenyl-3-carboxylic acid methylester;
4-Ethoxy-[1,1′;3′,1″]terphenyl-3-carboxylic acid [1-hydroxymethyl-2-(1H-indol-3-yl)ethyl]amide;
3′-Acetyl-4-ethoxybiphenyl-3-carboxylic acid [(R)-1-hydroxymethyl-2-(1H-indol-3-yl)ethyl]amide;
4-Ethoxy-3′-pyrrolidin-1-ylbiphenyl-3-carboxylic acid [(R)-1-hydroxymethyl-2-(1H-indol-3-yl)ethyl]amide;
4′-Cyanomethyl-4-ethoxybiphenyl-3-carboxylic acid [(R)-1-hydroxymethyl-2-(1H-indol-3-yl)ethyl]amide;
4′-Dimethylamino-4-propoxybiphenyl-3-carboxylic acid [(R)-1-hydroxymethyl-2-(1H-indol-3-yl)ethyl]amide;
4′-Hydroxymethyl-4-propoxybiphenyl-3-carboxylic acid [(R)-1-hydroxymethyl-2-(1H-indol-3-yl)ethyl]amide;
4-Propoxybiphenyl-3,4′-dicarboxylic acid 4′-diethylamide 3-{[(R)-1-hydroxymethyl-2-(1H-indol-3-yl)ethyl]amide};
3′-[(R)-1-Hydroxymethyl-2-(1H-indol-3-yl)ethylcarbamoyl]-4′-propoxybiphenyl-4-carboxylic acid;
4′-Acetyl-4-propoxybiphenyl-3-carboxylic acid [(R)-1-hydroxymethyl-2-(1H-indol-3-yl)ethyl]amide;
4′-Ethanesulphonyl-4-propoxybiphenyl-3-carboxylic acid [(R)-2-hydroxy-1-(1H-indol-3-ylmethyl)ethyl]amide;
3′-Cyanomethyl-4-propoxybiphenyl-3-carboxylic acid [(R)-1-hydroxymethyl-2-(1H-indol-3-yl)ethyl]amide;
3′-Methanesulphonylamino-4-propoxybiphenyl-3-carboxylic acid [(R)-2-hydroxy-1-(1H-indol-3-ylmethyl)ethyl]amide;
3′-Cyclopropylmethoxy-4-propoxybiphenyl-3-carboxylic acid [(R)-1-hydroxymethyl-2-(1H-indol-3-yl)ethyl]amide;
3′-Methanesulphonyl-4-propoxybiphenyl-3-carboxylic acid [(R)-2-hydroxy-1-(1H-indol-3-ylmethyl)ethyl]amide;
4-Propoxybiphenyl-3,3′-dicarboxylic acid 3′-[(2-dimethylaminoethyl)amide]3-{[(R)-1-hydroxymethyl-2-(1H-indol-3-yl)ethyl]amide};
3′-[(R)-1-Hydroxymethyl-2-(1H-indol-3-yl)ethylcarbamoyl]-3-methoxy-4′-propoxybiphenyl-4-carboxylic acid methyl ester;
3′-Chloro-4-propoxybiphenyl-3,4′-dicarboxylic acid 4′-amide 3-{[(R)-2-hydroxy-1-(1H-indol-3-ylmethyl)ethyl]amide};
3′-Dimethylsulphamoyl-4-propoxybiphenyl-3-carboxylic acid [(R)-2-hydroxy-1-(1H-indol-3-ylmethyl)ethyl]amide;
4′-(Propane-2-sulphonyl)-4-propoxybiphenyl-3-carboxylic acid [(R)-2-hydroxy-1-(1H-indol-3-ylmethyl)ethyl]amide;
4′-Methylsulphamoyl-4-propoxybiphenyl-3-carboxylic acid [(R)-2-hydroxy-1-(1H-indol-3-ylmethyl)ethyl]amide;
4′-Dimethylsulphamoyl-4-propoxybiphenyl-3-carboxylic acid [(R)-2-hydroxy-1-(1H-indol-3-ylmethyl)ethyl]amide;
4-Propoxybiphenyl-3,4′-dicarboxylic acid 4′-amide 3-{[(R)-1-hydroxymethyl-2-(1H-indol-3-yl)ethyl]amide};
3′-Methylsulphamoyl-4-propoxybiphenyl-3-carboxylic acid [(R)-2-hydroxy-1-(1H-indol-3-ylmethyl)ethyl]amide;
3′-Methanesulphonyl-4-propoxybiphenyl-3-carboxylic acid [(R)-2-hydroxy-1-(1-methyl-1H-indol-3-ylmethyl)ethyl]amide;
3′-[(R)-1-Hydroxymethyl-2-(1-methyl-1H-indol-3-yl)ethylcarbamoyl]-3-methoxy-4′-propoxybiphenyl-4-carboxylic acid methyl ester;
4-Propoxybiphenyl-3,4′-dicarboxylic acid 4′-[(2-dimethylaminoethyl)amide]3-{[(R)-1-hydroxymethyl-2-(1-methyl-1H-indol-3-yl)ethyl]amide};
3′-[2-(5-Fluoro-1H-indol-3-yl)-1-hydroxymethylethylcarbamoyl]-4′-propoxybiphenyl-4-carboxylic acid;
3′-Methanesulphonylamino-4-propoxybiphenyl-3-carboxylic acid [2-(5-fluoro-1H-indol-3-yl)-1-hydroxymethylethyl]amide;
3′-Methanesulphonyl-4-propoxybiphenyl-3-carboxylic acid [2-(5-fluoro-1H-indol-3-yl)-1-hydroxymethylethyl]amide;
4-Propoxybiphenyl-3,3′-dicarboxylic acid 3′-[(2-dimethylaminoethyl)amide]3-{[2-(5-fluoro-1H-indol-3-yl)-1-hydroxymethylethyl]amide};
3′-Chloro-4-propoxybiphenyl-3,4′-dicarboxylic acid 4′-amide 3-{[2-(5-fluoro-1H-indol-3-yl)-1-hydroxymethylethyl]amide};
3′-Dimethylsulphamoyl-4-propoxybiphenyl-3-carboxylic acid [2-(5-fluoro-1H-indol-3-yl)-1-hydroxymethylethyl]amide;
4′-(Propane-2-sulphonyl)-4-propoxybiphenyl-3-carboxylic acid [2-(5-fluoro-1H-indol-3-yl)-1-hydroxymethylethyl]amide;
4′-Dimethylsulphamoyl-4-propoxybiphenyl-3-carboxylic acid [2-(5-fluoro-1H-indol-3-yl)-1-hydroxymethylethyl]amide;
4-Propoxybiphenyl-3,4′-dicarboxylic acid 4′-diethylamide 3-{[2-(5-fluoro-1H-indol-3-yl)-1-hydroxymethylethyl]amide};
3′-Methylsulphamoyl-4-propoxybiphenyl-3-carboxylic acid [2-(5-fluoro-1H-indol-3-yl)-1-hydroxymethylethyl]amide;
3′-Acetyl-4-propoxybiphenyl-3-carboxylic acid [(R)-1-hydroxymethyl-2-(1-methyl-1H-indol-3-yl)ethyl]amide;
4-Propoxy-[1,1′;3′,1″]terphenyl-3-carboxylic acid [(R)-1-hydroxymethyl-2-(1-methyl-1H-indol-3-yl)ethyl]amide;
3′-Cyanomethyl-4-propoxybiphenyl-3-carboxylic acid [(R)-1-hydroxymethyl-2-(1-methyl-1H-indol-3-yl)ethyl]amide;
3′-Methanesulphonylamino-4-propoxybiphenyl-3-carboxylic acid [(R)-2-hydroxy-1-(1-methyl-1H-indol-3-ylmethyl)ethyl]amide;
4′-Cyanomethyl-4-propoxybiphenyl-3-carboxylic acid [(R)-1-hydroxymethyl-2-(1-methyl-1H-indol-3-yl)ethyl]amide;
4-Propoxybiphenyl-3,3′-dicarboxylic acid 3′-[(2-dimethylaminoethyl)amide]3-{[(R)-1-hydroxymethyl-2-(1-methyl-1H-indol-3-yl)ethyl]amide};
4-Fluoro-3′-[(R)-2-hydroxy-1-(1-methyl-1H-indol-3-ylmethyl)ethylcarbamoyl]-4′-propoxybiphenyl-3-carboxylic acid;
3′-Chloro-4-propoxybiphenyl-3,4′-dicarboxylic acid 4′-amide 3-{[(R)-2-hydroxy-1-(1-methyl-1H-indol-3-ylmethyl)ethyl]amide};
4-Propoxybiphenyl-3,4′-dicarboxylic acid 4′-diethylamide 3-{[(R)-1-hydroxymethyl-2-(1-methyl-1H-indol-3-yl)ethyl]amide};
4′-Dimethylamino-4-propoxybiphenyl-3-carboxylic acid [2-(5-fluoro-1H-indol-3-yl)-1-hydroxymethylethyl]amide;
4′-Acetyl-4-propoxybiphenyl-3-carboxylic acid [2-(5-fluoro-1H-indol-3-yl)-1-hydroxymethylethyl]amide;
3′-Acetyl-4-propoxybiphenyl-3-carboxylic acid [2-(5-fluoro-1H-indol-3-yl)-1-hydroxymethylethyl]amide;
4-Propoxy-[1,1′;3′,1″]terphenyl-3-carboxylic acid [2-(5-fluoro-1H-indol-3-yl)-1-hydroxymethylethyl]amide;
3′-[2-(5-Fluoro-1H-indol-3-yl)-1-hydroxymethylethylcarbamoyl]-3-methoxy-4′-propoxybiphenyl-4-carboxylic acid methyl ester;
4-Propoxybiphenyl-3,4′-dicarboxylic acid 4′-amide 3-{[2-(5-fluoro-1H-indol-3-yl)-1-hydroxymethylethyl]amide};
4-Ethoxy-4′-methoxymethylbiphenyl-3-carboxylic acid [(R)-1-hydroxymethyl-2-(1H-indol-3-yl)ethyl]amide;
4-Ethoxybiphenyl-3,3′-dicarboxylic acid 3′-amide 3-{[(R)-1-hydroxymethyl-2-(1H-indol-3-yl)ethyl]amide};
4′-Ethanesulphonyl-4-ethoxybiphenyl-3-carboxylic acid [(R)-2-hydroxy-1-(1H-indol-3-ylmethyl)ethyl]amide;
4-Ethoxy-4′-(4-methylpiperazine-1-carbonyl)biphenyl-3-carboxylic acid [(R)-1-hydroxymethyl-2-(1H-indol-3-yl)ethyl]amide;
3′-Cyclopropylmethoxy-4-ethoxybiphenyl-3-carboxylic acid [(R)-1-hydroxymethyl-2-(1H-indol-3-yl)ethyl]amide;
3′-[(R)-1-Hydroxymethyl-2-(1H-indol-3-yl)ethylcarbamoyl]biphenyl-2-carboxylic acid methyl ester.

9. Process for preparing compounds of the formula I according to claim 1, characterized in that tryptophanol derivatives of the formula VI 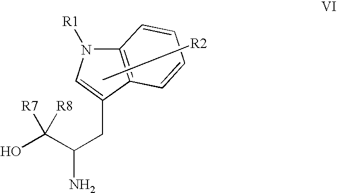
are coupled to carboxylic acids of the formula VII 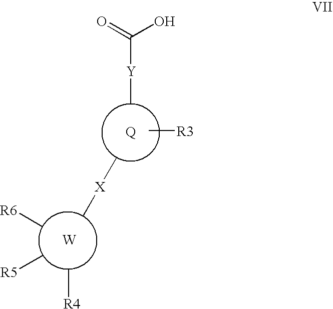
in an amide-formation reaction.

10. Process for preparing compounds of the formula Ia according to claim 2, characterized in that tryptophanol derivatives of the formula VIa 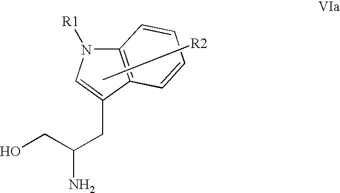
are coupled with carboxylic acids of the formula VII 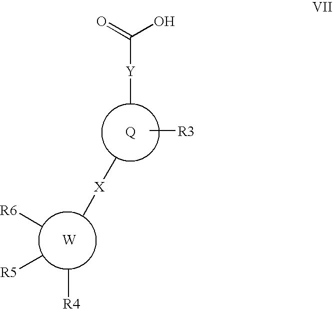
in an amide-formation reaction.

11. Process according to claim 9 for preparing compounds of the formulae II or III, characterized in that carboxylic acids of the formulae VIII or IX 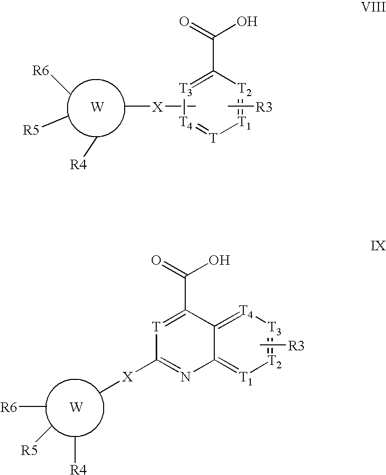
are employed.

12. Process according to claim 10 for preparing compounds of the formulae Ia or IIIa, characterized in that carboxylic acids of the formulae VIII or IX 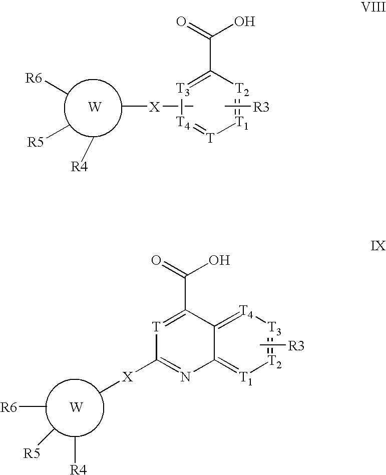
are employed.

13. Process according to claim 9 for preparing compounds of the formulae IV or V, characterized in that carboxylic acids of the formulae X or XI 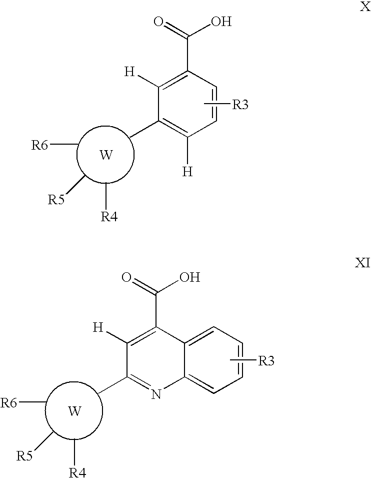
are employed.

14. Process according to claim 10 for preparing compounds of the formulae IVa or Va, characterized in that carboxylic acids of the formulae X or XI 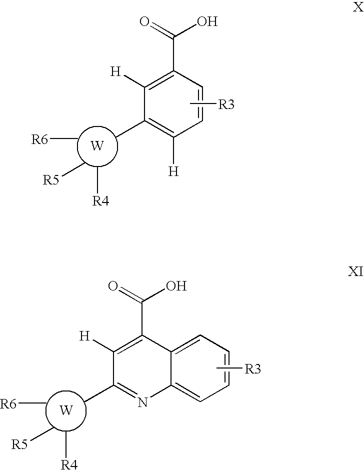
are employed.

15. Carboxylic acids according to claim 9, namely
2-(4-Chloro-3-methylphenyl)quinoline-4-carboxylic acid;
6-Methoxy-2-(3,4,5-trimethoxyphenyl)quinoline-4-carboxylic acid;
6-Methoxy-2-(2,3,4-trimethoxyphenyl)quinoline-4-carboxylic acid;
6-Fluoro-2-(3,4,5-trimethoxyphenyl)quinoline-4-carboxylic acid;
6-Iodo-2-(3,4,5-trimethoxyphenyl)quinoline-4-carboxylic acid;
6-Nitro-2-(3,4,5-trimethoxyphenyl)quinoline-4-carboxylic acid;
2-[4-(Trifluoromethoxy)phenyl]quinoline-4-carboxylic acid;
2-(3,5-dimethoxyphenyl)quinoline-4-carboxylic acid;
2-[(E)-2-(3,4-dimethoxyphenyl)ethenyl]-6-methoxyquinoline-4-carboxylic acid;
2′,3′,4′-Trimethoxy[1,1′-biphenyl]-3-carboxylic acid;
3′,4′,5′-Trimethoxy[1,1′-biphenyl]-4-carboxylic acid;
3′,4′,5′-Trimethoxy-2-methyl[1,1′-biphenyl]-4-carboxylic acid;
2′,3′,4′-Trimethoxy-6-methyl[1,1′-biphenyl]-3-carboxylic acid;
2′,3′,4′-Trimethoxy[1,1′-biphenyl]-4-carboxylic acid;
2′,3′,4′-Trimethoxy-2-methyl[1,1′-biphenyl]-4-carboxylic acid;
3′,4,4′,5′-Tetramethoxy[1,1′-biphenyl]-4-carboxylic acid;
4′-(Hydroxymethyl)-6-methyl[1,1′-biphenyl]-3-carboxylic acid;
4′-(Hydroxymethyl)-2-methyl[1,1′-biphenyl]-4-carboxylic acid;
4-methoxy-3′-(1-methylethyl)[1,1′-biphenyl]-2-carboxylic acid;
3′-(1-methylethyl)[1,1′-biphenyl]-3-carboxylic acid;
6-methyl-3′-(1-methylethyl)[1,1′-biphenyl]-3-carboxylic acid;
3′-(1-methylethyl)[1,1′-biphenyl]-4-carboxylic acid;
2-methyl-3′-(1-methylethyl)[1,1′-biphenyl]-4-carboxylic acid;
4′-(Hydroxymethyl)-4-methoxy[1,1′-biphenyl]-2-carboxylic acid;
3′,4′,5′-Trifluoro[1,1′-biphenyl]-2-carboxylic acid;
3′,4′,5′-Trifluoro[1,1′-biphenyl]-3-carboxylic acid;
3′,4′,5′-Trifluoro-6-methyl[1,1′-biphenyl]-3-carboxylic acid;
3′,4′,5′-Trifluoro[1,1′-biphenyl]-4-carboxylic acid;
3′,4′,5′-Trifluoro-2-methyl[1,1′-biphenyl]-4-carboxylic acid;
2′,4,5′-Trimethoxy[1,1′-biphenyl]-2-carboxylic acid;
2′,4,5′-Trimethoxy[1,1′-biphenyl]-2-carboxylic acid;
2′,5′-dimethoxy[1,1′-biphenyl]-4-carboxylic acid;
2′,5′-dimethoxy-2-methyl[1,1′-biphenyl]-4-carboxylic acid;
3′,4,4′-Trimethoxy[1,1′-biphenyl]-2-carboxylic acid;
3′,4′-dimethoxy-6-methyl[1,1′-biphenyl]-2-carboxylic acid;
3′,4′-dimethoxy-2-methyl[1,1′-biphenyl]-4-carboxylic acid;
3′-Fluoro-4′-methoxy[1,1′-biphenyl]-2-carboxylic acid;
3′-Fluoro-4,4′-dimethoxy[1,1′-biphenyl]-2-carboxylic acid;
3′-Fluoro-4′-methoxy[1,1′-biphenyl]-3-carboxylic acid;
3′-Fluoro-4′-methoxy-6-methyl[1,1′-biphenyl]-3-carboxylic acid;
3′-Fluoro-4′-methoxy-2-methyl[1,1′-biphenyl]-4-carboxylic acid;
3′,4′-dimethoxy[1,1′-biphenyl]-2-carboxylic acid;
3′-(1-methylethyl)[1,1′-biphenyl]-2-carboxylic acid;
2′,5′-dimethoxy[1,1′-biphenyl]-3-carboxylic acid;
3′,4′,5′-Trifluoro-4-methoxy[1,1′-biphenyl]-2-carboxylic acid;
3-(Benzofuran-2-yl)benzoic acid;
3-(5-methoxybenzofuran-2-yl)benzoic acid;
2-[(3,4,5-Trimethoxyphenyl)methoxy]phenylpropanoic acid;
4-[[(3,4,5-Trimethoxyphenyl)methoxy]methyl]benzoic acid;
3-[(3,4,5-Trimethoxyphenyl)methoxy]thiophene-2-carboxylic acid;
4-[(3,4,5-Trimethoxyphenyl)methoxy]phenylacetic acid;
3-[3-((3,4,5-Trimethoxyphenyl)methoxy)phenyl]propionic acid;
2-[(E)-2-(3,4-dimethoxyphenyl)ethenyl]-6-methoxyquinoline-4-carboxylic acid;
and their methyl, ethyl, propyl and butyl esters.

16. Carboxylic acids according to claim 9, namely
2-(4-Fluoro-3-methoxyphenyl)-6-methoxyquinoline-4-carboxylic acid;
2-(3-Iodo-4-methoxyphenyl)-6-methoxyquinoline-4-carboxylic acid;
2-(3-Hydroxyphenyl)-6-methoxyquinoline-4-carboxylic acid;
2-(4-Hydroxy-3,5-dimethoxyphenyl)-6-methoxyquinoline-4-carboxylic acid;
2-(3,5-Difluoro-4-methoxyphenyl)-6-methoxyquinoline-4-carboxylic acid;
2-(3-Ethylphenyl)-6-methoxyquinoline-4-carboxylic acid;
2-(3-Fluoro-4-methoxyphenyl)-6-methoxyquinoline-4-carboxylic acid;
2-(3-Fluoro-4-methoxyphenyl)-6-methylquinoline-4-carboxylic acid;
6-Methyl-2-(3,4,5-trimethoxyphenyl)quinoline-4-carboxylic acid;
6-Bromo-2-(2,4-dimethylthiazol-5-yl)quinoline-4-carboxylic acid;
2-(7-Methoxybenzofuran-2-yl)-6-trifluoromethoxyquinoline-4-carboxylic acid;
2-(3-Fluoro-4-methoxyphenyl)-6-iodoquinoline-4-carboxylic acid;
2-(3-Fluoro-4-methoxyphenyl)-6-trifluoromethoxyquinoline-4-carboxylic acid;
2-(3-Fluoro-4-methoxyphenyl)-6,8-dimethylquinoline-4-carboxylic acid;
2-(3,4-Dimethoxyphenyl)-6-methoxy-3-methylquinoline-4-carboxylic acid;
2-(4,6-Dimethoxybenzofuran-2-yl)-6-methoxyquinoline-4-carboxylic acid;
6-Methoxy-2-(5-methoxybenzofuran-2-yl)quinoline-4-carboxylic acid;
2-(7-Ethoxybenzofuran-2-yl)-6-methoxyquinoline-4-carboxylic acid;
6-Methoxy-2-(6-methoxybenzofuran-2-yl)quinoline-4-carboxylic acid;
2-(7-Fluorbenzofuran-2-yl)-6-methoxyquinoline-4-carboxylic acid;
2-(4-Fluorbenzofuran-2-yl)-6-methoxyquinoline-4-carboxylic acid;
6-Methoxy-2-(5-methylbenzofuran-2-yl)quinoline-4-carboxylic acid;
6-Methoxy-2-(7-methylbenzofuran-2-yl)quinoline-4-carboxylic acid;
6-Methoxy-2-(4-methoxybenzofuran-2-yl)quinoline-4-carboxylic acid;
6-Methoxy-2-[5-(trifluoromethoxy)benzofuran-2-yl]quinoline-4-carboxylic acid;
5-Bromo-2-ethoxy-N-[(R)-2-hydroxy-1-(1H-indol-3-ylmethyl)ethyl]benzamide;
N-[(R)-1-Hydroxymethyl-2-(1H-indol-3-yl)ethyl]-5-iod-2-propoxybenzamide;
N-[(R)-1-Hydroxymethyl-2-(1-methyl-1H-indol-3-yl)ethyl]-5-iod-2-propoxybenzamide;
N-[2-(5-Fluoro-1H-indol-3-yl)-1-hydroxymethylethyl]-5-iod-2-propoxybenzamide
4-Ethoxy-3′-fluoro-4′-methoxy[1,1′-biphenyl]-3-carboxylic acid;
4-Ethoxy-3′-methoxy[1,1′-biphenyl]-3-carboxylic acid;
4-Ethoxy-3′-[(methylamino)carbonyl][1,1′-biphenyl]-3-carboxylic acid;
4-Ethoxy-3′,4′,5′-trimethoxy[1,1′-biphenyl]-3-carboxylic acid;
4-Ethoxy-3′,4′-dimethoxy[1,1′-biphenyl]-3-carboxylic acid;
4-Ethoxy-3′-(1-methylethyl)[1,1′-biphenyl]-3-carboxylic acid;
3′,4′,5′-Trimethoxy-4-propoxy[1,1′-biphenyl]-3-carboxylic acid;
3′,4′-Dimethoxy-4-propoxy[1,1′-biphenyl]-3-carboxylic acid;
3′-Methoxy-4-propoxy[1,1′-biphenyl]-3-carboxylic acid;
3′-[(Methylamino)carbonyl]-4-propoxy[1,1′-biphenyl]-3-carboxylic acid;
4,3′,4′,5′-Tetramethoxybiphenyl-3-carboxylic acid;
4,3′,4′-Trimethoxybiphenyl-3-carboxylic acid;
3′-Fluoro-4,4′-dimethoxybiphenyl-3-carboxylic acid;
4,3′-Dimethoxybiphenyl-3-carboxylic acid;
5-Benzo[1,3]dioxol-5-yl-2-methoxybenzoic acid;
3′,4′-Difluoro-4,5′-dimethoxybiphenyl-3-carboxylic acid;
4-Isopropoxy-3′-methoxybiphenyl-3-carboxylic acid;
5-Benzo[1,3]dioxol-5-yl-2-isopropoxybenzoic acid;
4-Isopropoxy-3′,4′,5′-trimethoxybiphenyl-3-carboxylic acid;
3′-Fluoro-4-isopropoxy-4′-methoxybiphenyl-3-carboxylic acid;
4-Isopropoxy-3′,4′-dimethoxybiphenyl-3-carboxylic acid;
4-Isopropoxy-3′-methylbiphenyl-3-carboxylic acid;
4′-Fluoro-4-isopropoxy-3′-methylbiphenyl-3-carboxylic acid;
3′,4′-Difluoro-4-isopropoxy-5′-methoxybiphenyl-3-carboxylic acid;
4,3′,4′,5′-Tetramethoxy-5-methylbiphenyl-3-carboxylic acid;
4,3′,4′-Trimethoxy-5-methylbiphenyl-3-carboxylic acid;
3′-Fluoro-4,4′-dimethoxy-5-methylbiphenyl-3-carboxylic acid;
5-Benzo[1,3]dioxol-5-yl-2-methoxy-3-methylbenzoic acid;
4,3′-Dimethoxy-5-methylbiphenyl-3-carboxylic acid;
4-Methoxy-5,3′-dimethylbiphenyl-3-carboxylic acid;
4′-Fluoro-4-methoxy-5,3′-dimethylbiphenyl-3-carboxylic acid;
3′,4′-Difluoro-4,5′-dimethoxy-5-methylbiphenyl-3-carboxylic acid;
3′-Hydroxy-4-isopropoxybiphenyl-3-carboxylic acid;
3′,4′,5′-Trimethoxy-4-(3-methylbut-2-enyloxy)biphenyl-3-carboxylic acid;
5-(7-Methoxybenzofuran-2-yl)-2-propoxybenzoic acid;
6-Methoxy-2-(3,4,5-trimethoxyphenyl)quinoline-4-carboxylic acid;
N-[(R)-1-(Methoxycarbonyl)-2-(1-ethyl)-1H-indol-3-yl)ethyl]-6-methoxy-2-(3-methoxyphenyl)quinoline-4-carboxamide;
N-[(R)-1-(Methoxycarbonyl)-2-(1-propyl-1H-indol-3-yl)ethyl]-6-methoxy-2-(3,4,5-trimethoxyphenyl)quinoline-4-carboxamide;
N-[(R)-1-(Methoxycarbonyl)-2-(1-ethyl)-1H-indol-3-yl)ethyl]-2-(3,5-difluoro-4-methoxyphenyl)-6-methoxyquinoline-4-carboxamide;
N-[(R)-1-(Methoxycarbonyl)-2-(1-isopropyl-1H-indol-3-yl)ethyl]-6-methoxy-2 (3-methoxyphenyl)-quinoline-4-carboxamide;
N-[(R)-1-(Methoxycarbonyl)-2-(1-isopropyl-1H-indol-3-yl)ethyl]-2-(3,5-difluoro-4-methoxyphenyl)-6-methoxyquinoline-4-carboxamide;
N-[(R)-1-(Methoxycarbonyl)-2-(1-isopropyl-1H-indol-3-yl)ethyl]-6-methoxy-2-(3,4,5-trimethoxyphenyl)quinoline-4-carboxamide;
N-[(R)-1-(Methoxycarbonyl)-2-(1-ethyl)-1H-indol-3-yl)ethyl]-2-(3-fluoro-4-methoxyphenyl)-6-trifluoromethoxyquinoline-4-carboxamide;
N-[(R)-1-(Methoxycarbonyl)-2-(1-ethyl)-1H-indol-3-yl)ethyl]-2-(7-methoxybenzofuran-2-yl)-6-trifluoromethoxyquinoline-4-carboxamide;
N-[(R)-1-(Methoxycarbonyl)-2-(1-isopropyl-1H-indol-3-yl)ethyl]-2-(3-fluoro-4-methoxyphenyl)-6-trifluoromethoxyquinoline-4-carboxamide;
N-[(R)-1-(Methoxycarbonyl)-2-(1-isopropyl-1H-indol-3-yl)ethyl]-2-(7-methoxybenzofuran-2-yl)-6-trifluormethoxy-quinoline-4-carboxamide;
N-[(R)-1-(Methoxycarbonyl)-2-(1-n-hexyl-1H-indol-3-yl)ethyl]-6-methoxy-2-(3,4,5-trimethoxyphenyl)quinoline-4-carboxamide;
N-[(R)-1-(Methoxycarbonyl)-2-(1-ethyl)-1H-indol-3-yl)ethyl]-4-ethoxy-3′-methoxybiphenyl-3-carboxamide;
N-[(R)-1-(Methoxycarbonyl)-2-(1-isopropyl)-1H-indol-3-yl)ethyl]-4-ethoxy-3′-methoxybiphenyl)-3-carboxamide;
6-Bromoquinoline-8-carboxylic acid [(R)-1-hydroxymethyl-2-(1H-indol-3-yl)ethyl]amide;
3-Bromonaphthalene-1-carboxylic acid [(R)-1-hydroxymethyl-2-(1H-indol-3-yl)ethyl]amide;
5-Bromo-4-methoxythiophene-3-carboxylic acid [(R)-2-hydroxy-1-(1H-indol-3-ylmethyl)ethyl]amide;
6-Bromo-1H-benzimidazole-4-carboxylic acid [(R)-2-hydroxy-1-(1H-indol-3-ylmethyl)ethyl]amide;
2-(3,4,5-Trimethoxyphenyl)thiazol-4-carboxylic acid;
5-(3,4,5-Trimethoxyphenyl)thiophene-2-carboxylic acid;
5-(3,4,5-Trimethoxyphenyl)benzo[b]thiophene-2-carboxylic acid;
2-(3-Fluoro-4-methoxyphenyl)-6-methylisonicotinic acid;
2-(3-Fluoro-4-methoxyphenyl)-6-methylpyrimidine-4-carboxylic acid;
6-(4-Methoxyphenyl)-pyrimidine-4-carboxylic acid;
2-(3-Fluoro-4-methoxyphenyl)-6-methoxyquinazoline-4-carboxylic acid;
2-(3-Fluoro-4-methoxyphenyl)-6-iodoquinazoline-4-carboxylic acid;
2-(4-methoxyphenyl)-quinazoline-4-carboxylic acid;
4-Hydroxy-3′,4′,5′-trimethoxybiphenyl-3-carboxylic acid;
4-(3-Cyanopropoxy)-3′,4′,5′-trimethoxybiphenyl-3-carboxylic acid;
4-Cyclopentyloxy-3′-fluoro-4′-methoxybiphenyl-3-carboxylic acid;
4-Cyclopentyloxy-3′-methylbiphenyl-3-carboxylic acid;
3′-(1-Butyl-3-methylureido)-4-cyclopentyloxybiphenyl-3-carboxylic acid;
4-Cyclopentyloxy-4′-fluoro-3′-methylbiphenyl-3-carboxylic acid;
4-Cyclopentyloxy-3′-methoxybiphenyl-3-carboxylic acid;
4-Cyclopentyloxy-3′,4′-dimethoxybiphenyl-3-carboxylic acid;
5-Benzo[1,3]dioxol-5-yl-2-cyclopentyloxybenzoic acid;
4-Cyclopentyloxy-3′,4′,5′-trimethoxybiphenyl-3-carboxylic acid;
4-Cyclopentyloxy-3′,4′-difluoro-5′-methoxybiphenyl-3-carboxylic acid;
3′-[Butyl[(1,1-dimethylethoxy)carbonyl]amino]-4-propoxy[1,1′-biphenyl]-3-carboxylic acid;
3′-(1-Butyl-3-methylureido)-4-methoxybiphenyl-3-carboxylic acid;
3′-(1-Butyl-3-methylureido)-4-methoxy-5-methylbiphenyl-3-carboxylic acid;
3′-(1-Butyl-3-methylureido)-4-isopropoxybiphenyl-3-carboxylic acid
and their methyl, ethyl, propyl and butyl esters.

17. Pharmaceutical compositions comprising one or more of the compounds according to claim 1 with pharmacologically suitable recipients and carriers.

18. Use of the compounds of the general formula I according to claim 1 for fertility control in men or in women.

19. Process for producing medicaments comprising one or more of the compounds of the general formula I according to claim 1 for the prevention and/or treatment of osteoporosis.
